(19) United States
(12) Patent Application Publication
(10) Pub. No.: US 2023/0391787 A1
(43) Pub. Date: Dec. 7, 2023

(54) ORGANIC MOLECULE LIGHT EMITTERS

(71) Applicant: The Governing Council of the University of Toronto, Toronto (CA)

(72) Inventors: Robert Pollice, Toronto (CA); Alan Aspuru-Guzik, Toronto (CA); Pascal Friederich, Neckarsulm (DE); Cyrille Lavigne, Toronto (CA); Gabriel Dos Passos Gomes, Toronto (CA)

(21) Appl. No.: 18/030,634

(22) PCT Filed: Oct. 8, 2021
(86) PCT No.: PCT/CA2021/051423

§ 371 Date: Apr. 6, 2023

Related U.S. Application Data
(60) Provisional application No. 63/090,024, filed on Oct. 9, 2020.

(51) Int. Cl. C07D 487/16; C07D 471/16; C07F 7/08; H10K 50/12; H01S 5/36; C07D 495/22; C09K 11/06

(52) U.S. Cl. CPC C07D 487/16; C07D 471/16; C07F 7/0812; H10K 50/12; H01S 5/36; C07D 495/22; C09K 11/06

(57) ABSTRACT

The present application relates to compounds of Formula I having a negative singlet-triplet gap and a positive oscillator strength. The present application also relates to use of the compounds of Formula (I) in photocatalysis and in OLEDs as emitters and/or dopants.

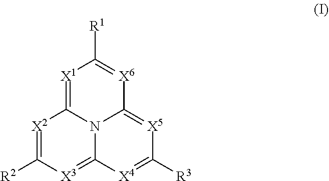

### CROSS-REFERENCE TO RELATED APPLICATIONS

The present application claims the benefit of priority from U.S. patent application No. 63/090,024, filed Oct. 9, 2020, the contents of which are incorporated herein by reference in their entirety.

### STATEMENT REGARDING FEDERALLY SPONSORED RESEARCH

This invention was made with government support under Contract No. HR00111920027 awarded by Defense Advanced Research Projects Agency (DARPA). The government has certain rights in the invention.

### FIELD

The present application relates to organic compounds with a negative singlet-triplet gap and a positive oscillator strength. The present application further relates to the use of the compounds as emitters and/or dopants in organic light-emitting diodes (OLED) and in photocatalysis.

### INTRODUCTION

The design of state-of-the-art organic light-emitting diodes (OLEDs) has focused mainly on molecules consisting of spatially separated but electronically connected, donor and acceptor π-systems. Accordingly, their low-lying electronic excited states are typically of significant charge-transfer character minimizing the associated exchange energy difference leading to vanishing singlet-triplet gaps. This feature allows facile upconversion of excited state triplets to excited state singlets via thermally activated delayed fluorescence (TADF) resulting in OLEDs with internal quantum efficiencies (IQEs) of up to 100% and external quantum efficiencies (EQEs) rivaling those of state-of-the-art organometallic OLEDs. However, the large-scale market deployment of TADF-based OLEDs remains limited, due to a lack of blue and red emitters, of TADF molecules possessing color purity, and of devices with long-term operational stability.

Hund's first rule (1) predicts that the first excited state of closed-shell molecules is a triplet state lower in energy than the first excited singlet state. This prediction holds for all but a handful of all known organic and inorganic compounds. (2,3) Hence, it is the basis for Jablonski diagrams (4) in educational material about electronic spectra of molecules illustrating that it is almost considered a basic truth in chemistry. (5-12) Accordingly, molecules violating Hund's first rule in their first excited singlet and triplet energies, i.e. molecules with excited state triplet(s) higher in energy than excited state singlet(s), are said to possess an “inverted” singlet-triplet gap (herein termed the INVEST property). Very few organic INVEST molecules were predicted previously to exist based on computations alone (2, 17, 18) with little to no experimental evidence (19, 20) and no inorganic INVEST molecule is known to date. Besides inherent INVEST molecules, it has been shown in recent years that the influence of the environment can also invert the gap (13) for instance in exciplexes (14) through strong light-matter coupling in microcavities (15) and polarizable environments. (16)

Nevertheless, recent publications spark new interest in INVEST molecules and their potential applications in photocatalysis, and organic optoelectronics as emissive layer in organic light-emitting diodes (OLEDs). (21, 22) The two molecules reported were both based on phenalene (23) with a distinct degree of nitrogen substitution. However, both molecules have dipole-forbidden S₁-S₀ transitions (due to spatial symmetry) and are likely very poor emitters.

Accordingly, there is a need to develop organic INVEST molecules.

### SUMMARY

Molecules with appreciable oscillator strength and inverted singlet-triplet gaps have the potential to become the next generation of OLED materials (13, 24) because of their potential for fast reverse intersystem crossing (i.e., TADF without activation), high emission rates, and a thermodynamic equilibrium that disfavors triplets, and, hence, minimizes triplet annihilation and nonradiative Ti decay processes that shorten device lifetimes. (13)

Based on computational evidence, in the present application, it has been shown that compounds of the present application exhibit appreciable oscillator strength. Overall, it was observed that the singlet-triplet gap, the oscillator strength, and the absorption wavelength can be tuned by modification, including nitrogen substitution, of the phenalene core. It was also observed that the compounds of the present application, azaphenalenes substituted with electron-donating and electron-withdrawing substituents, have increased oscillator strength but still an inverted singlet-triplet gap. Equally, systematic optimization of substituted azaphenalenes was investigated for high oscillator strength, small singlet-triplet gap, and absorption wavelength leading to compounds of the present application with considerable oscillator strength, covering the visible light spectrum.

Accordingly, in one aspect, the present application includes a compound of Formula I

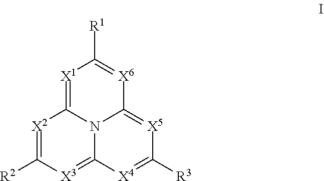

wherein
X¹ is selected from N and CR⁴;
X² is selected from N and CR⁵;
X³ is selected from N and CR⁶;
X⁴ is selected from N and CR⁷;
X⁵ is selected from N and CR⁸;
X⁶ is selected from N and CR⁹;
provided that at least one, but not all, of X¹-X⁶ is N;
R¹-R⁹ are independently selected from H, halo, NO₂, CN, isonitrile, C(O)H, NH₂, OH, SH, C(O)NH₂, C₁₋₁₀alkyl, C₃₋₁₀cycloalkyl, C₂₋₁₀alkenyl, C₂₋₁₀alkynyl, OC₁₋₁₀alkyl, NHC₁₋₁₀alkyl, NH(C₃₋₁₀cycloalkyl), N(C₁₋₁₀alkyl)(C₁₋₁₀alkyl), 3- to 8-membered heterocycle, C(O)C₁₋₁₀alkyl, CO₂C₁₋₁₀alkyl, C(O)NHC₁₋₁₀alkyl, C(O)N(C₁₋₁₀alkyl)(C₁₋₁₀alkyl), SC₁₋₁₀alkyl, S(O)C₁₋₁₀alkyl, SO₂C₁₋₁₀alkyl, OC(O)C₁₋₁₀alkyl, NHC(O)C₁₋₁₀alkyl, aryl, O-aryl, NH-aryl, N(aryl)(aryl), S-aryl, S(O)-aryl, OSO₂C₁₋₁₀alkyl, SO₂-aryl, C(O)-aryl; CO₂-aryl, C(O)NH-aryl, OC(O)-aryl, NHC(O)-aryl, heteroaryl, O-heteroaryl, NH-heteroaryl, S-heteroaryl, S(O)-heteroaryl, SO₂-heteroaryl, C(O)-heteroaryl, C(O)NH₂, CO₂-heteroaryl, C(O)NH— heteroaryl, OC(O)C₁₋₁₀alkyl, OC(O)-heteroaryl and NHC(O)-heteroaryl, wherein all alkyl, cycloalkyl, alkenyl, alkynyl, aryl, heterocycle, and heteroaryl groups are each unsubstituted or substituted with one or more substituents independently selected from R¹⁰;
or optionally, R¹ to R⁵, R⁸ and R⁹ are as defined above, R⁶ and R⁷ are linked to form X⁷═X⁸, which, together with X³, X⁴ and the carbon atom therebetween, form a five membered ring;
X⁷ is selected from N and CR¹¹;
X⁸ is selected from N and CR¹²;
optionally, R² and R¹¹ and/or R³ and R¹² together with the atoms therebetween are linked to form a 5- or 6-membered carbocycle or heterocycle, optionally an aromatic or heteroaromatic cycle, wherein the 5- or 6-membered carbocycle or heterocycle is unsubstituted or substituted with one or more substituents independently selected from R¹⁰;
or optionally, R¹, R⁴, R⁵, R⁸ and R⁹ are as defined above, R² and R⁶ and/or R³ and R⁷ together with the atoms therebetween are linked to form a 5- or 6-membered carbocycle or heterocycle, optionally an aromatic or heteroaromatic cycle, wherein the 5- or 6-membered carbocycle or heterocycle is unsubstituted or substituted with one or more substituents independently selected from R¹⁰;
R¹⁰ is selected from halo, NO₂, CN, isonitrile, C(O)H, NH₂, OH, SH, BH₂, C₁₋₆alkyl boronic ester, C₁₋₆alkyl borane, diaryl borane, C₂₋₆alkyldiol cyclic boronic ester, C(O)NH₂, C₃₋₁₀cycloalkyl, C₁₋₁₀alkyl, C₂₋₁₀alkenyl, C₂₋₁₀alkynyl, OC₁₋₁₀alkyl, NHC₁₋₁₀alkyl, N(C₁₋₁₀alkyl)(C₁₋₁₀alkyl), N(aryl)(aryl), NH(C₃₋₁₀cycloalkyl), 3- to 8-membered heterocycle, C(O)C₁₋₁₀alkyl, CO₂C₁₋₁₀alkyl, C(O)NHC₁₋₁₀alkyl, C(O)N(C₁₋₁₀alkyl)(C₁₋₁₀alkyl), SC₁₋₁₀alkyl, S(O)C₁₋₁₀alkyl, SO₂C₁₋₁₀alkyl, OC(O)C₁₋₁₀alkyl, NHC(O)H, NHC(O)C₁₋₁₀alkyl, aryl, O-aryl, NH-aryl, S-aryl, S(O)-aryl, SO₂-aryl, C(O)-aryl; CO₂-aryl, C(O)NH-aryl, OC(O)-aryl, NHC(O)-aryl, heteroaryl, O-heteroaryl, NH-heteroaryl, S-heteroaryl, S(O)-heteroaryl, SO₂-heteroaryl, C(O)-heteroaryl; CO₂-heteroaryl, C(O)NH-heteroaryl, OC(O)-heteroaryl and NHC(O)-heteroaryl, wherein all alkyl, cycloalkyl, alkenyl, alkynyl, aryl, heterocycle, and heteroaryl groups are each unsubstituted or substituted with one or more substituents independently selected from halo, NO₂, CN, NH₂, OH, C₃₋₁₀cycloalkyl, C₁₋₁₀alkyl, OC₁₋₁₀alkyl, NHC₁₋₁₀alkyl, N(C₁₋₁₀alkyl)(C₁₋₁₀alkyl), NH(C₃₋₁₀cycloalkyl), trialkylsilanyl, C(O)aryl, aryl, heteroaryl, O-heteroaryl, N-heteroaryl, and S-heteroaryl;
R¹¹ and R¹² are independently selected from H, halo, NO₂, CN, C(O)H, NH₂, OH, SH, C(O)NH₂, C₁₋₁₀alkyl, C₂₋₁₀alkenyl, C₂₋₁₀alkynyl, OC₁₋₁₀alkyl, NHC₁₋₁₀alkyl, N(C₁₋₁₀alkyl)(C₁₋₁₀alkyl), C(O)C₁₋₁₀alkyl, CO₂C₁₋₁₀alkyl, C(O)NHC₁₋₁₀alkyl, C(O)N(C₁₋₁₀alkyl)(C₁₋₁₀alkyl), SC₁₋₁₀alkyl, S(O)C₁₋₁₀alkyl, SO₂C₁₋₁₀alkyl, OC(O)C₁₋₁₀alkyl, NHC(O)C₁₋₁₀alkyl, aryl, O-aryl, NH-aryl, S-aryl, S(O)-aryl, SO₂-aryl, C(O)-aryl; CO₂-aryl, C(O)NH-aryl, OC(O)-aryl, NHC(O)-aryl, heteroaryl, O-heteroaryl, NH-heteroaryl, S-heteroaryl, S(O)-heteroaryl, SO₂-heteroaryl, C(O)-heteroaryl; CO₂-heteroaryl, C(O)NH-heteroaryl, OC(O)-heteroaryl and NHC(O)-heteroaryl, wherein all alkyl, alkenyl, alkynyl, aryl and heteroaryl groups are each unsubstituted or substituted with one or more substituents independently selected from R¹³;
R¹³ is selected from halo, NO₂, CN, isonitrile, C(O)H, NH₂, OH, SH, C(O)NH₂, C₁₋₁₀alkyl, C₂₋₁₀alkenyl, C₂₋₁₀alkynyl, OC₁₋₁₀alkyl, NHC₁₋₁₀alkyl, N(C₁₋₁₀alkyl)(C₁₋₁₀alkyl), C(O)C₁₋₁₀alkyl, CO₂C₁₋₁₀alkyl, C(O)NHC₁₋₁₀alkyl, C(O)N(C₁₋₁₀alkyl)(C₁₋₁₀alkyl), SC₁₋₁₀alkyl, S(O)C₁₋₁₀alkyl, SO₂C₁₋₁₀alkyl, OC(O)C₁₋₁₀alkyl, NHC(O)C₁₋₁₀alkyl, aryl, O-aryl, NH-aryl, S-aryl, S(O)-aryl, SO₂-aryl, C(O)-aryl; CO₂-aryl, C(O)NH-aryl, OC(O)-aryl, NHC(O)-aryl, heteroaryl, O-heteroaryl, NH-heteroaryl, S-heteroaryl, S(O)-heteroaryl, SO₂-heteroaryl, C(O)-heteroaryl; CO₂-heteroaryl, C(O)NH-heteroaryl, OC(O)-heteroaryl and NHC(O)-heteroaryl;
all available H atoms are each optionally fluoro-substituted;
wherein the compound has a negative singlet-triple gap and an oscillator strength greater than or equal to about 0.01.

In another aspect, the present application includes an organic light-emitting diode comprising at least one compound of the present application.

In another aspect, the present application includes a photocatalyst comprising at least one compound of the present application.

In another aspect, the present application includes a triplet quencher comprising at least one compound of the present application.

In another aspect, the present application also includes a use of a compound of the present application in an organic light-emitting diode.

In another aspect, the present application also includes a method of preparing an organic light-emitting diode comprising providing at least one compound of the present application as an emitter or a dopant.

In another aspect, the present application includes a use of a compound of the present application as a photocatalysis.

In another aspect, the present application includes a method of performing photocatalysis comprising providing at least one compound of the present application as a photocatalyst.

In another aspect, the present application includes a use of a compound of the present application in the generation of organic laser.

In another aspect, the present application includes a method of generating organic laser comprising providing at least one compound of the present application as a light emitter.

In another aspect, the present application includes a use of a compound of the present application in the enhancement of photostability.

In another aspect, the present application includes a method of enhancing photostability comprising providing at least one compound of the present application as a triplet quencher.

### DRAWINGS

The embodiments of the application will now be described in greater detail with reference to the attached drawings in which:

FIG. 1 shows a plot of oscillator strength (f₁₂) and singlet-triplet gap of exemplary azaphenalene compounds with different nitrogen substitution as shown in Scheme 2.

FIG. 2 shows a plot of oscillator strength (f₁₂) and singlet-triplet gap of exemplary azaphenalene compounds 1-6 with different monosubstitution as shown in Scheme 4.

FIG. 3 shows benchmarking of computational methods for singlet-triplet gaps in Panel A and oscillator strength in Panel B.

FIG. 4 shows in Panel A the singlet-triplet gap and oscillator strength in y-axes of each exemplary compound computed in Example 5 (compound number in x-axis), and in Panel B for a plot of oscillator strength vs singlet-triplet gap of the exemplary compounds.

FIG. 5 shows maps of singlet-triplet gaps, oscillator strengths in Panel A and vertical excitation energies in Panel B of different nitrogen-substitution of CH in exemplary azacyclopenta[cd]phenalene 18 as shown in Scheme 5 at the EOM-CCSD/cc-pVDZ level of theory. The horizontal gray line in Panel B indicates a vertical excitation energy of 2.85 eV corresponding to about 468 nm, after correcting for the solvatochromic shift.

FIG. 6 shows maps of singlet-triplet gaps, oscillator strengths and vertical excitation energies of exemplary monosubstituted analogues of compound 21 as shown in Scheme 7 at the EOM-CCSD/cc-pVDZ level of theory. The diamond-shaped data point corresponds to exemplary unsubstituted compound 21.

FIG. 7 shows properties of different exemplary substituted analogues of compound 21. Panel A shows singlet-triplet gap and oscillator strength. Panel B shows vertical S₁ and T₁ excitation energies. Panels C and D show property maps of all exemplary compounds investigated during the optimization, aiming at potential blue INVEST emitters. Notable structures are marked with diamond markers (Panels A to D) and diamond-shaped markers outlines (Panels C and D) respectively. The horizontal gray line in (b) and (d) indicates a vertical excitation energy of 3.2 eV corresponding to about 448 nm, after correcting for the solvatochromic shift.

FIG. 8 shows a plot of oscillator strength of exemplary minimal analogues of INVEST molecules shown in Scheme 8 using benchmark quality methods in Panel A and comparison of the molecules' vertical and adiabatic singlet-triplet gaps in Panel B. Data points with diamond-shaped contour correspond to the corresponding unsubstituted cores 3-6.

FIG. 9 shows a plot comparing vertical and adiabatic singlet-triplet gaps from ωB2PLYP′ calculations for the benchmark dataset in Example 10.

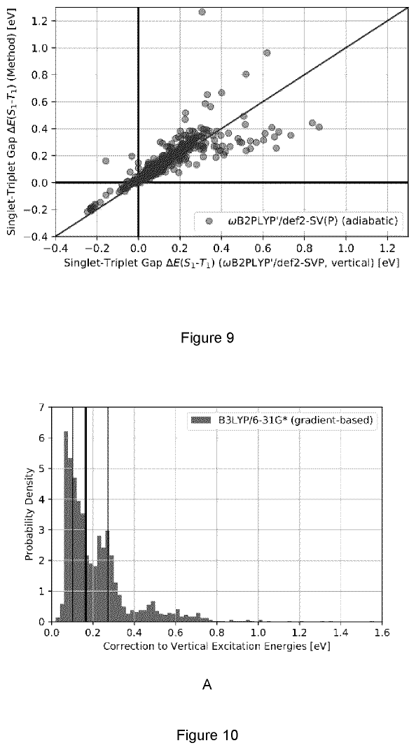

FIG. 10 shows the impact of excited state geometry relaxation on spectroscopic properties. Panel A shows a histogram of differences of vertical excitation energy and emission energy across all compounds investigated in Example 10. Vertical lines in Panel A indicate first, second and third quantiles, respectively. Panel B shows comparison of fluorescence rate estimates from the absorption oscillator strength and the gradient-based approach.

FIG. 11 shows validation of minimal analogues of INVEST molecules with appreciable fluorescence rates. in a device environment using implicit solvent models. By comparing singlet-triplet gaps in Panel A and oscillator strengths in Panel B with and without C-PCM at the ωB2PLYP′/def2-SVP level of theory. Data points with lighter colors correspond to the corresponding unsubstituted cores 3-6.

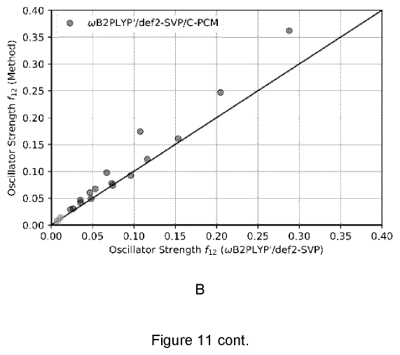

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DESCRIPTION OF VARIOUS EMBODIMENTS

### I. Definitions

Unless otherwise indicated, the definitions and embodiments described in this and other sections are intended to be applicable to all embodiments and aspects of the present application herein described for which they are suitable as would be understood by a person skilled in the art.

The term “compound(s) of the application” or “compound(s) of the present application” and the like as used herein refers to a compound of Formula I.

The term “and/or” as used herein means that the listed items are present, or used, individually or in combination. In effect, this term means that “at least one of” or “one or more” of the listed items is used or present.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds of the present application may also exist in different tautomeric forms and it is intended that any tautomeric forms which the compounds form, as well as mixtures thereof, are included within the scope of the present application.

The compounds of the present application may further exist in varying polymorphic forms and it is contemplated that any polymorphs, or mixtures thereof, which form are included within the scope of the present application.

The present description refers to a number of chemical terms and abbreviations used by those skilled in the art. Nevertheless, definitions of selected terms are provided for clarity and consistency.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms.

The term “alkenyl” as used herein, whether it is used alone or as part of another group, means straight or branched chain, unsaturated alkyl groups containing at least one double bond. The number of carbon atoms that are possible in the referenced alkylene group are indicated by the prefix “Cₙ₁₋ₙ₂”. For example, the term C₂₋₆alkenyl means an alkenyl group having 2, 3, 4, 5 or 6 carbon atoms and at least one double bond.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms.

The term “cycloalkyl,” as used herein, whether it is used alone or as part of another group, means a saturated carbocyclic group containing from 3 to 20 carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms.

The term “aryl” as used herein, whether it is used alone or as part of another group, refers to carbocyclic groups containing at least one aromatic ring and contains either 6 to 20 carbon atoms.

The term “heterocycloalkyl” as used herein, whether it is used alone or as part of another group, refers to cyclic groups containing at least one non-aromatic ring containing from 3 to 20 atoms in which one or more of the atoms are a heteroatom selected from O, S and N and the remaining atoms are C. Heterocycloalkyl groups are either saturated or unsaturated (i.e. contain one or more double bonds). When a heterocycloalkyl group contains the prefix Cₙ₁₋ₙ₂ this prefix indicates the number of carbon atoms in the corresponding carbocyclic group, in which one or more, suitably 1 to 5, of the ring atoms is replaced with a heteroatom as selected from O, S and N and the remaining atoms are C. Heterocycloalkyl groups are optionally benzofused.

The term “heteroaryl” as used herein, whether it is used alone or as part of another group, refers to cyclic groups containing at least one heteroaromatic ring containing 5-20 atoms in which one or more of the atoms are a heteroatom selected from O, S and N and the remaining atoms are C. When a heteroaryl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above. Heteroaryl groups are optionally benzofused.

The term “heterocycle” as used herein, whether it is used alone or as a part of another group, refers to cyclic groups containing at least one heterocycloalkyl ring or at least one heteroaromatic ring.

All cyclic groups, including aryl, heteroaryl, heterocycloalkyl and cycloalkyl groups, contain one or more than one ring (i.e. are polycyclic). When a cyclic group contains more than one ring, the rings may be fused, bridged, spirofused or linked by a bond.

The term “benzofused” as used herein refers to a polycyclic group in which a benzene ring is fused with another ring.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “fluorosubstituted” refers to the substitution of one or more, including all, available hydrogens in a referenced group with fluoro.

The terms “halo” or “halogen” as used herein, whether it is used alone or as part of another group, refers to a halogen atom and includes fluoro, chloro, bromo and iodo.

The term “available”, as in “available hydrogen atoms” or “available atoms” refers to atoms that would be known to a person skilled in the art to be capable of replacement by a substituent.

The term “amine” or “amino,” as used herein, whether it is used alone or as part of another group, refers to groups of the general formula NR′R″, wherein R′ and R″ are each independently selected from hydrogen or C₁₋₁₀alkyl.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

### II. Compounds and Compositions of the Application

In one aspect, the present application includes a compound of Formula I

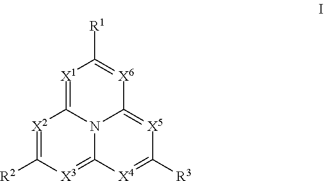

wherein
X¹ is selected from N and CR⁴;
X² is selected from N and CR⁵;
X³ is selected from N and CR⁶;
X⁴ is selected from N and CR⁷;
X⁵ is selected from N and CR⁸;
X⁶ is selected from N and CR⁹;
provided that at least one, but not all, of X¹-X⁶ is N;
R¹-R⁹ are independently selected from H, halo, NO₂, CN, isonitrile, C(O)H, NH₂, OH, SH, C(O)NH₂, C₁₋₁₀alkyl, C₃₋₁₀cycloalkyl, C₂₋₁₀alkenyl, C₂₋₁₀alkynyl, OC₁₋₁₀alkyl, NHC₁₋₁₀alkyl, NH(C₃₋₁₀cycloalkyl), N(C₁₋₁₀alkyl)(C₁₋₁₀alkyl), 3- to 8-membered heterocycle, C(O)C₁₋₁₀alkyl, CO₂C₁₋₁₀alkyl, C(O)NHC₁₋₁₀alkyl, C(O)N(C₁₋₁₀alkyl)(C₁₋₁₀alkyl), SC₁₋₁₀alkyl, S(O)C₁₋₁₀alkyl, SO₂C₁₋₁₀alkyl, OC(O)C₁₋₁₀alkyl, NHC(O)C₁₋₁₀alkyl, aryl, O-aryl, NH-aryl, N(aryl)(aryl), S-aryl, S(O)-aryl, OS₂C₁₋₁₀alkyl, SO₂-aryl, C(O)-aryl; CO₂-aryl, C(O)NH-aryl, OC(O)-aryl, NHC(O)-aryl, heteroaryl, O-heteroaryl, NH-heteroaryl, S-heteroaryl, S(O)-heteroaryl, SO₂-heteroaryl, C(O)-heteroaryl, C(O)NH₂, CO₂-heteroaryl, C(O)NH— heteroaryl, OC(O)C₁₋₁₀alkyl, OC(O)-heteroaryl and NHC(O)-heteroaryl, wherein all alkyl, cycloalkyl, alkenyl, alkynyl, aryl, heterocycle, and heteroaryl groups are each unsubstituted or substituted with one or more substituents independently selected from R¹⁰;
or optionally, R¹ to R⁵, R⁸ and R⁹ are as defined above, R⁶ and R⁷ are linked to form X⁷═X⁸, which, together with X³, X⁴ and the carbon atom therebetween, form a five membered ring;
X⁷ is selected from N and CR¹¹;
X⁸ is selected from N and CR¹²
optionally, R² and R¹¹ and/or R³ and R¹² together with the atoms therebetween are linked to form a 5- or 6-membered carbocycle or heterocycle, optionally an aromatic or heteroaromatic cycle, wherein the 5- or 6-membered carbocycle or heterocycle is unsubstituted or substituted with one or more substituents independently selected from R¹⁰;
or optionally, R¹, R⁴, R⁵, R⁸ and R⁹ are as defined above, R² and R⁶ and/or R³ and R⁷ together with the atoms therebetween are linked to form a 5- or 6-membered carbocycle or heterocycle, optionally an aromatic or heteroaromatic cycle, wherein the 5- or 6-membered carbocycle or heterocycle is unsubstituted or substituted with one or more substituents independently selected from R¹⁰;
R¹⁰ is selected from halo, NO₂, CN, isonitrile, C(O)H, NH₂, OH, SH, BH₂, C₁₋₆alkyl boronic ester, C₁₋₆alkyl borane, diaryl borane, C₂₋₆alkyldiol cyclic boronic ester, C(O)NH₂, C₃₋₁₀cycloalkyl, C₁₋₁₀alkyl, C₂₋₁₀alkenyl, C₂₋₁₀alkynyl, OC₁₋₁₀alkyl, NHC₁₋₁₀alkyl, N(C₁₋₁₀alkyl)(C₁₋₁₀alkyl), N(aryl)(aryl), NH(C₃₋₁₀cycloalkyl), 3- to 8-membered heterocycle, C(O)C₁₋₁₀alkyl, CO₂C₁₋₁₀alkyl, C(O)NHC₁₋₁₀alkyl, C(O)N(C₁₋₁₀alkyl)(C₁₋₁₀alkyl), SC₁₋₁₀alkyl, S(O)C₁₋₁₀alkyl, SO₂C₁₋₁₀alkyl, OC(O)C₁₋₁₀alkyl, NHC(O)H, NHC(O)C₁₋₁₀alkyl, aryl, O-aryl, NH-aryl, S-aryl, S(O)-aryl, SO₂-aryl, C(O)-aryl; CO₂-aryl, C(O)NH-aryl, OC(O)-aryl, NHC(O)-aryl, heteroaryl, O-heteroaryl, NH-heteroaryl, S-heteroaryl, S(O)-heteroaryl, SO₂-heteroaryl, C(O)-heteroaryl; CO₂-heteroaryl, C(O)NH-heteroaryl, OC(O)-heteroaryl and NHC(O)-heteroaryl, wherein all alkyl, cycloalkyl, alkenyl, alkynyl, aryl, heterocycle, and heteroaryl groups are each unsubstituted or substituted with one or more substituents independently selected from halo, NO₂, CN, NH₂, OH, C₃₋₁₀cycloalkyl, C₁₋₁₀alkyl, OC₁₋₁₀alkyl, NHC₁₋₁₀alkyl, N(C₁₋₁₀alkyl)(C₁₋₁₀alkyl), NH(C₃₋₁₀cycloalkyl), trialkylsilanyl, C(O)aryl, aryl, heteroaryl, O-heteroaryl, N-heteroaryl, and S-heteroaryl;
R¹¹ and R¹² are independently selected from H, halo, NO₂, CN, C(O)H, NH₂, OH, SH, C(O)NH₂, C₁₋₁₀alkyl, C₂₋₁₀alkenyl, C₂₋₁₀alkynyl, OC₁₋₁₀alkyl, NHC₁₋₁₀alkyl, N(C₁₋₁₀alkyl)(C₁₋₁₀alkyl), C(O)C₁₋₁₀alkyl, CO₂C₁₋₁₀alkyl, C(O)NHC₁₋₁₀alkyl, C(O)N(C₁₋₁₀alkyl)(C₁₋₁₀alkyl), SC₁₋₁₀alkyl, S(O)C₁₋₁₀alkyl, SO₂C₁₋₁₀alkyl, OC(O)C₁₋₁₀alkyl, NHC(O)C₁₋₁₀alkyl, aryl, O-aryl, NH-aryl, S-aryl, S(O)-aryl, SO₂-aryl, C(O)-aryl; CO₂-aryl, C(O)NH-aryl, OC(O)-aryl, NHC(O)-aryl, heteroaryl, O-heteroaryl, NH-heteroaryl, S-heteroaryl, S(O)-heteroaryl, SO₂-heteroaryl, C(O)-heteroaryl; CO₂-heteroaryl, C(O)NH-heteroaryl, OC(O)-heteroaryl and NHC(O)-heteroaryl, wherein all alkyl, alkenyl, alkynyl, aryl and heteroaryl groups are each unsubstituted or substituted with one or more substituents independently selected from R¹³;
R¹³ is selected from halo, NO₂, CN, isonitrile, C(O)H, NH₂, OH, SH, C(O)NH₂, C₁₋₁₀alkyl, C₂₋₁₀alkenyl, C₂₋₁₀alkynyl, OC₁₋₁₀alkyl, NHC₁₋₁₀alkyl, N(C₁₋₁₀alkyl)(C₁₋₁₀alkyl), C(O)C₁₋₁₀alkyl, CO₂C₁₋₁₀alkyl, C(O)NHC₁₋₁₀alkyl, C(O)N(C₁₋₁₀alkyl)(C₁₋₁₀alkyl), SC₁₋₁₀alkyl, S(O)C₁₋₁₀alkyl, SO₂C₁₋₁₀alkyl, OC(O)C₁₋₁₀alkyl, NHC(O)C₁₋₁₀alkyl, aryl, O-aryl, NH-aryl, S-aryl, S(O)-aryl, SO₂-aryl, C(O)-aryl; CO₂-aryl, C(O)NH-aryl, OC(O)-aryl, NHC(O)-aryl, heteroaryl, O-heteroaryl, NH-heteroaryl, S-heteroaryl, S(O)-heteroaryl, SO₂-heteroaryl, C(O)-heteroaryl; CO₂-heteroaryl, C(O)NH-heteroaryl, OC(O)-heteroaryl and NHC(O)-heteroaryl;
all available H atoms are each optionally fluoro-substituted;
wherein the compound has a negative singlet-triple gap and an oscillator strength greater than or equal to about 0.01.

In some embodiments, the oscillator strength is greater than or equal to about 0.03. In some embodiments, the oscillator strength is greater than or equal to about 0.05. In some embodiments, the oscillator strength is greater than or equal to about 0.1. In some embodiments, the oscillator strength is greater than or equal to about 0.2. In some embodiments, the oscillator strength is greater than or equal to about 0.3. In some embodiments, the oscillator strength is greater than or equal to about 0.4. In some embodiments, the oscillator strength is greater than or equal to about 0.5. In some embodiments, the oscillator strength is greater than or equal to about 0.6. In some embodiments, the oscillator strength is greater than or equal to about 0.7. In some embodiments, the oscillator strength is greater than or equal to about 0.8. In some embodiments, the oscillator strength is greater than or equal to about 0.9. In some embodiments, the oscillator strength is greater than or equal to about 1.

In some embodiments, R¹ and R⁹ are not all H.

In some embodiments, 2 to 4 of X¹ to X⁶ are N

In some embodiments, each halo is independently selected from F, Br, and Cl.

In some embodiments, each C₁₋₁₀alkyl is independently selected from linear and branched C₁₋₆alkyl. In some embodiments, the linear and branched C₁₋₆alkyl is selected from methyl, ethyl, propyl, butyl, isopropyl, secpropyl, secbutyl, and tertbutyl.

In some embodiments, each heterocycle and heterocyclocycloalkyl is independently selected from azetidine, aziridine, pyrrolidine, pipperidine, morpholine, tetrahydrofuran, tetrahydropyran, tetrahydrothiopyran, indolinone, and quinolinone.

In some embodiments, each aryl is independently selected from phenyl and naphthyl. In some embodiments, each aryl is phenyl.

In some embodiments, each heterocycle and heteroaryl is independently selected from pyrrole, pyrazole, pyridine, indole, carbazole, indazole, imidazole, oxazole, isoxazole, thiazole, thiophene, furan, pyridazine, isothiazole, pyrimidine, benzofuran, benzothiophene, benzoimidazole, and quinoline.

In some embodiments, R¹-R⁹ are independently selected from H, F, Br, Cl, NO₂, CN, isonitrile, C(O)H, NH₂, OH, SH, C₁₋₆alkyl, C₃₋₈cycloalkyl, C₂₋₄alkenyl, C₂₋₄alkynyl, OC₁₋₆alkyl, NHC₁₋₆alkyl, N(C₁₋₆alkyl)(C₁₋₆alkyl), C(O)C₁₋₆alkyl, SC₁₋₆alkyl, S(O)C₁₋₆alkyl, OC(O)C₁₋₆alkyl, aryl, N(aryl)(aryl), S-aryl, heteroaryl, C(O)NH₂. In some embodiments, R¹-R⁹ are independently selected from H, F, Br, Cl, NO₂, CN, isonitrile, C(O)H, NH₂, OH, SH, CF₃, methyl, ethyl, propyl, butyl, isopropyl, secpropyl, secbutyl, tertbutyl, C₃₋₆cycloalkyl, CH═CH₂, C≡CH, OCH₃, OEt, Oisopropyl, Otertbutyl, OCF₃, NHCH₃, NHCH₂CH₃, NHisopropyl, NHtertbutyl, N(CH₃)₂, NH(CH₂CH₃)₂, C(O)CH₃, C(O)CH₂CH₃, SCH₃, SCH₂CH₃, S(O)CH₃, S(O)CH₂CH₃, OC(O)CH₃, OC(O)CH₂CH₃, phenyl, naphthyl, N(phenyl)(phenyl), S-phenyl, S-naphthyl, NH-phenyl, O-pehynl, pyrrole, pyrazole, indole, indazole, benzoimidazole, pyridine, carbazole, benzofuran, benzothiophene, furan, thiophene, imidazole, oxazole, isoxazole, thiazole, C(O)NH₂.

In some embodiments, R¹⁰ is selected from F, Br, Cl, NO₂, CN, NH₂, OH, SH, C₁₋₆alkyl, OC₁₋₆alkyl, NHC₁₋₆alkyl, N(C₁₋₆alkyl)(C₁₋₆alkyl), N(aryl)(aryl), NH(C₃₋₁₀cycloalkyl), 3- to 8-membered heterocycloalkyl, NHC(O)H, NHC(O)C₁₋₆alkyl, aryl, NH-aryl, C(O)-aryl, heteroaryl, NH-heteroaryl, wherein all alkyl, cycloalkyl, alkenyl, alkynyl, aryl, C₁₋₁₀akyl substituted aryl, heterocycle, and heteroaryl groups are each unsubstituted or substituted with one or more substituents independently selected from halo, NO₂, CN, NH₂, OH, C₃₋₆cycloalkyl, C₁₋₆alkyl, OC₁₋₆alkyl, N(C₁₋₆alkyl)(C₁₋₆alkyl), trialkylsilanyl, heteroaryl.

In some embodiments, R¹⁰ is selected from F, Br, Cl, NO₂, CN, NH₂, OH, SH, CF₃, methyl, ethyl, propyl, butyl, isopropyl, secpropyl, secbutyl, tertbutyl, OCH₃, OEt, Oisopropyl, Otertbutyl, OCF₃, NHCH₃, NHCH₂CH₃, NHisopropyl, NHtertbutyl, N(CH₃)₂, N(isopropyl)₂, N(phenyl)(phenyl), NH(C₃₋₆cycloalkyl), azetidine, aziridine, pyrrolidine, pipperidine, morpholine, tetrahydrofuran, tetrahydropyran, tetrahydrothiopyran, NHC(O)H, NHC(O)CH₃, NHC(O)CH₂CH₃, phenyl, naphthyl, NH-phenyl, NH-naphthyl, C(O)-phenyl, pyrrole, imidazole, pyrazole, carbazole, indole, NH-pyridine, NH-pyrrole, NH-furan, NH-imidazole, NH-thiophene, NH-pyridazine, NH-pyrimidine, NH-isoxazole, NH-oxazole, NH-pyrazole, NH-isothiazole, NH-thiazole, NH-indole, wherein all alkyl, cycloalkyl, alkenyl, alkynyl, aryl, heterocycle, and heteroaryl groups are each unsubstituted or substituted with one or more substituents independently selected from F, NO₂, CN, NH₂, OH, C₃₋₆cycloalkyl, methyl, ethyl, propyl, butyl, isopropyl, secpropyl, secbutyl, tertbutyl, OCH₃, OEt, N(CH₃)₂, N(CH₂CH₃)₂, triethylsilanyl, trimethylsilanyl phenyl, pyrazine.

In some embodiments, the compound of the present application is selected from

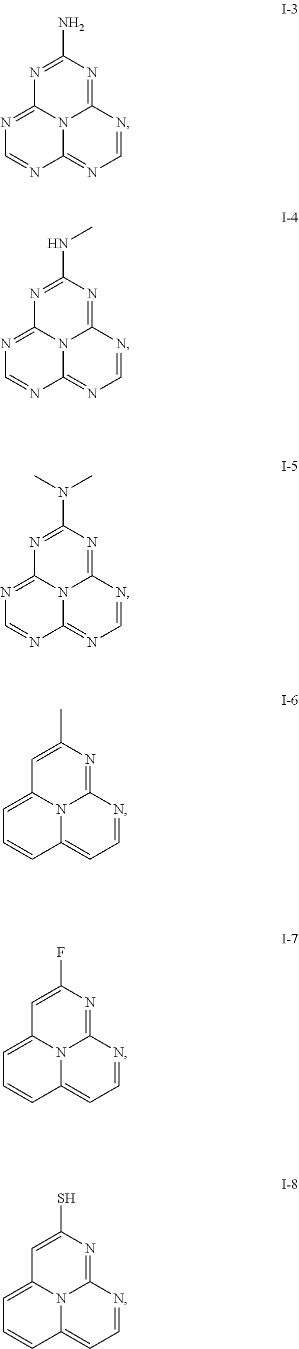 [SMILES: NC1=NC2=NC=NC3=NC=NC(=N1)N32 ; CNC1=NC2=NC=NC3=NC=NC(=N1)N32 ; CN(C)C1=NC2=NC=NC3=NC=NC(=N1)N32 ; CC1=CC2=CC=CC3=CC=NC(=N1)N32 ; FC1=CC2=CC=CC3=CC=NC(=N1)N32 ; SC1=CC2=CC=CC3=CC=NC(=N1)N32]

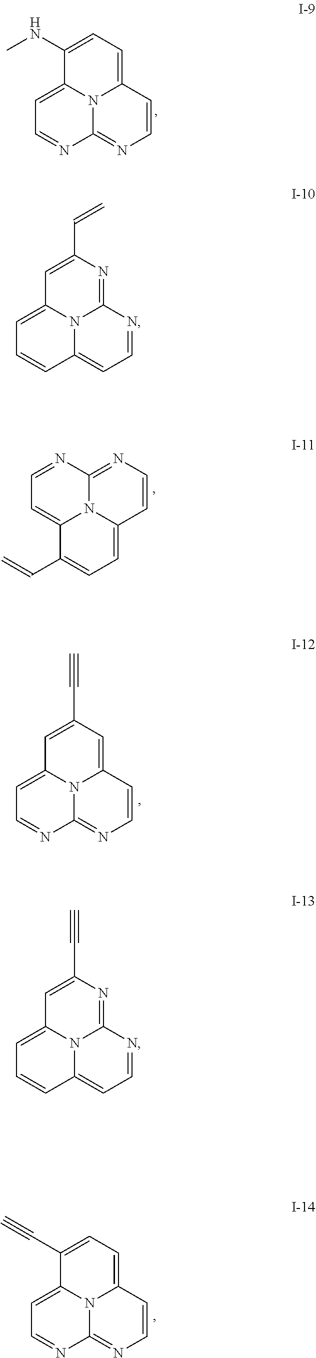

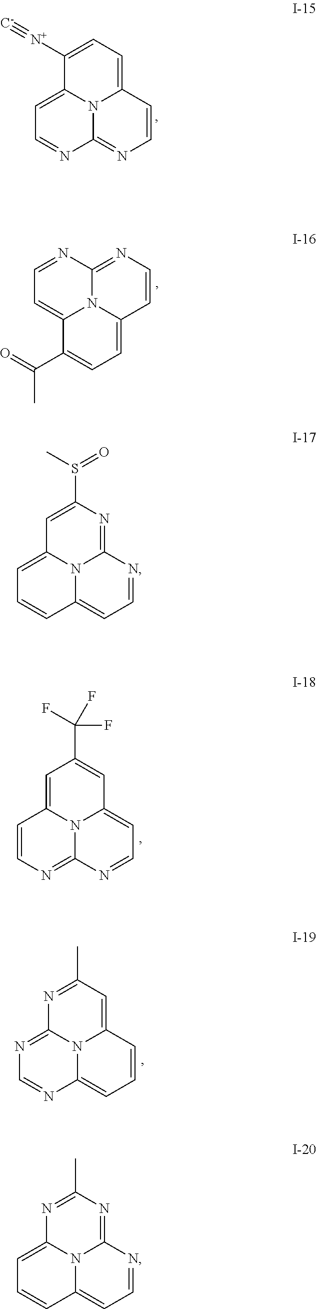 [SMILES: [C-]#[N+]C1=CC=C2C=CN=C3N=CC=C1N23 ; CC(=O)C1=CC=C2C=CN=C3N=CC=C1N23 ; CS(=O)C1=CC2=CC=CC3=CC=NC(=N1)N32 ; FC(F)(F)C1=CC2=CC=NC3=NC=CC(=C1)N23 ; CC1=NC2=NC=NC3=CC=CC(=C1)N32 ; CC1=NC2=CC=CC3=CC=NC(=N1)N32]

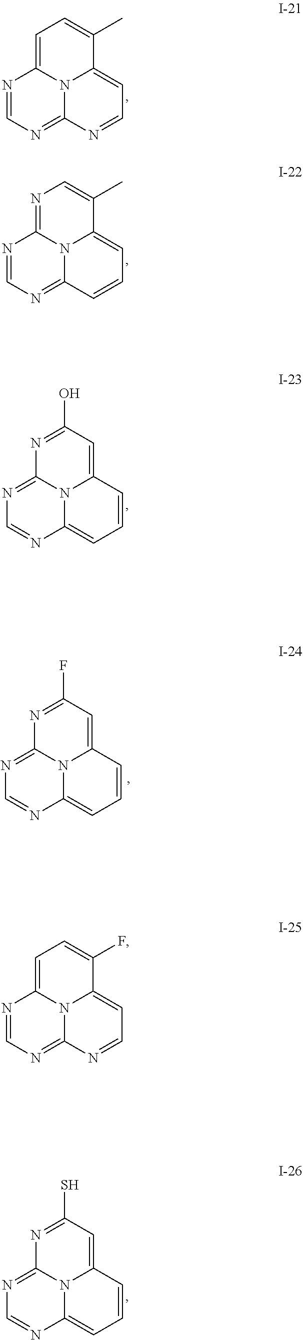

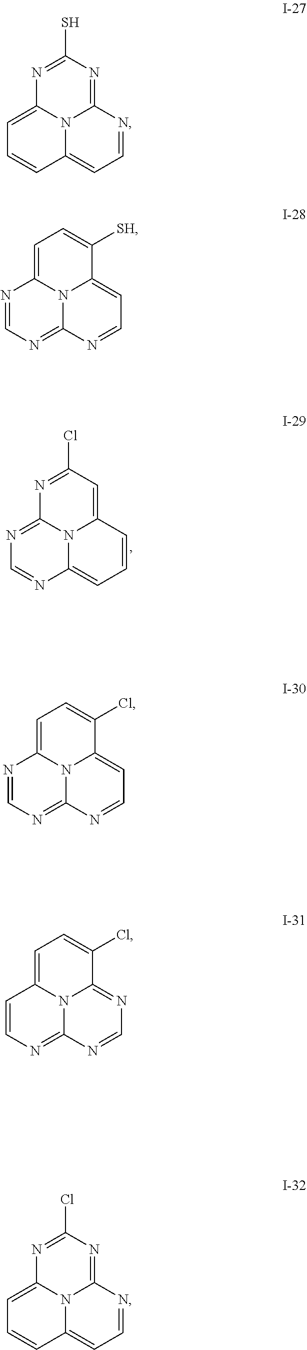 [SMILES: SC1=NC2=CC=CC3=CC=NC(=N1)N32 ; SC1=CC=C2N=CN=C3N=CC=C1N23 ; ClC1=NC2=NC=NC3=CC=CC(=C1)N32 ; ClC1=CC=C2N=CN=C3N=CC=C1N23 ; ClC1=CC=C2C=CN=C3N=CN=C1N23 ; ClC1=NC2=CC=CC3=CC=NC(=N1)N32]

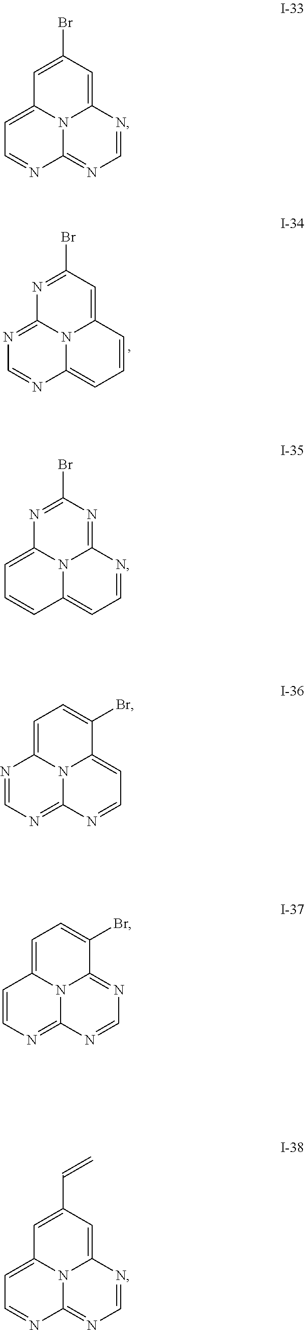

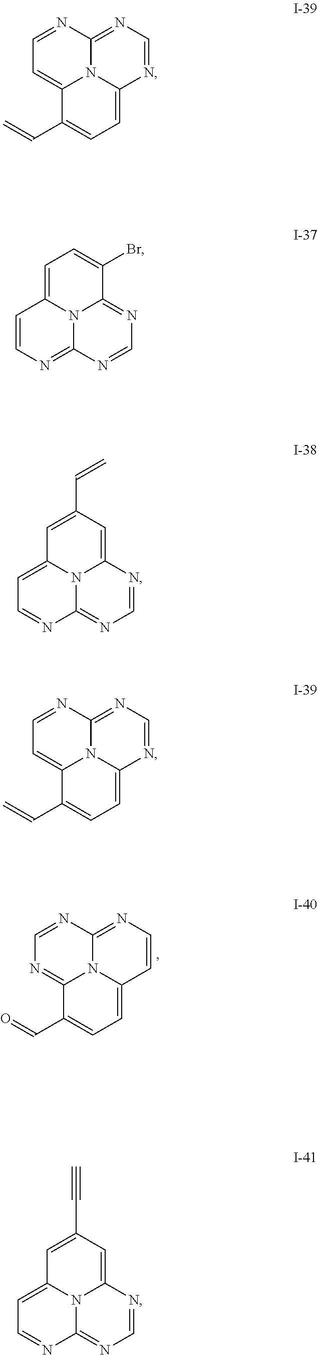

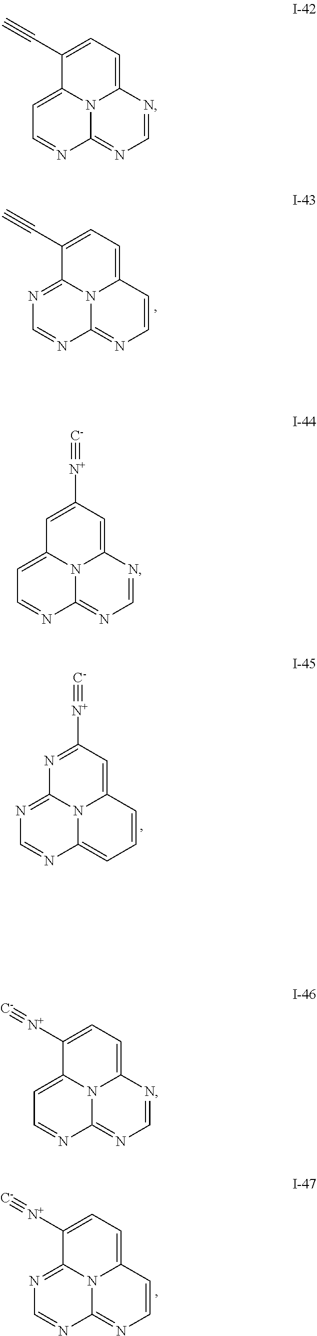

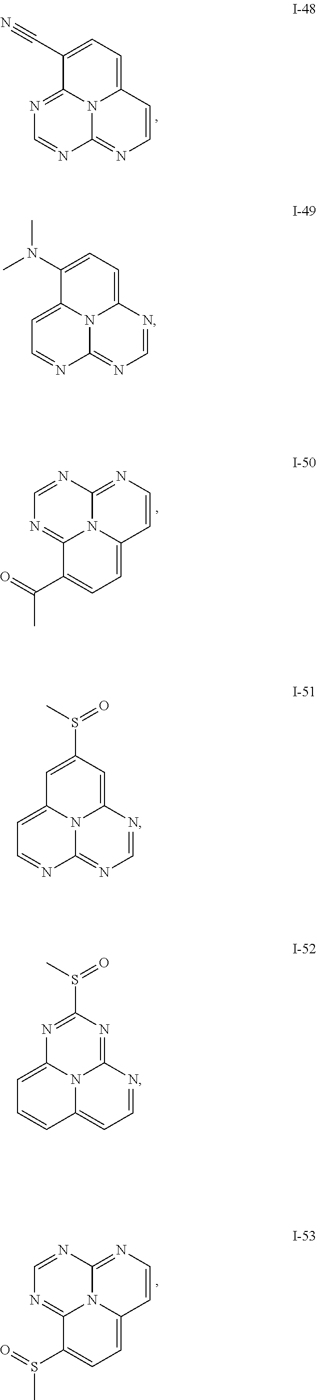 [SMILES: N#CC1=CC=C2C=CN=C3N=CN=C1N23 ; CN(C)C1=CC=C2N=CN=C3N=CC=C1N23 ; CC(=O)C1=CC=C2C=CN=C3N=CN=C1N23 ; CS(=O)C1=CC2=CC=NC3=NC=NC(=C1)N23 ; CS(=O)C1=NC2=CC=CC3=CC=NC(=N1)N32 ; CS(=O)C1=CC=C2C=CN=C3N=CN=C1N23]

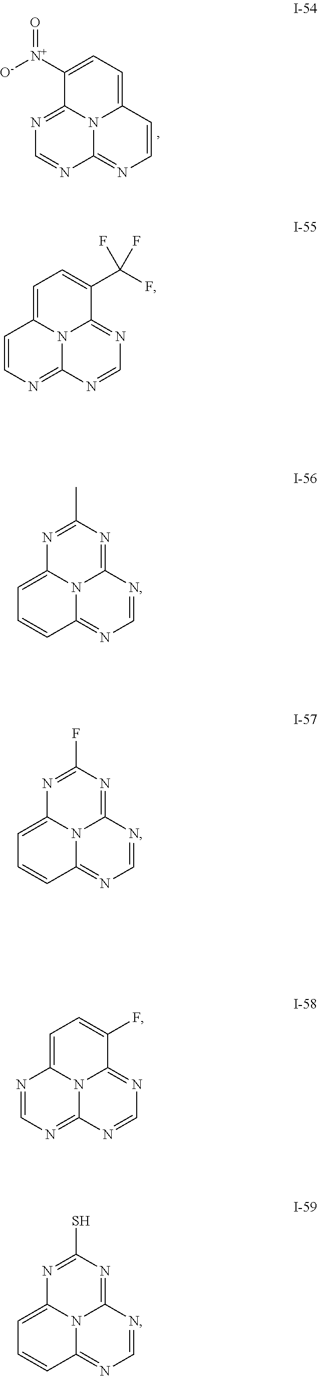 [SMILES: O=[N+]([O-])C1=CC=C2C=CN=C3N=CN=C1N23 ; FC(F)(F)C1=CC=C2C=CN=C3N=CN=C1N23 ; CC1=NC2=CC=CC3=NC=NC(=N1)N23 ; FC1=NC2=CC=CC3=NC=NC(=N1)N23 ; FC1=CC=C2N=CN=C3N=CN=C1N23 ; SC1=NC2=CC=CC3=NC=NC(=N1)N23]

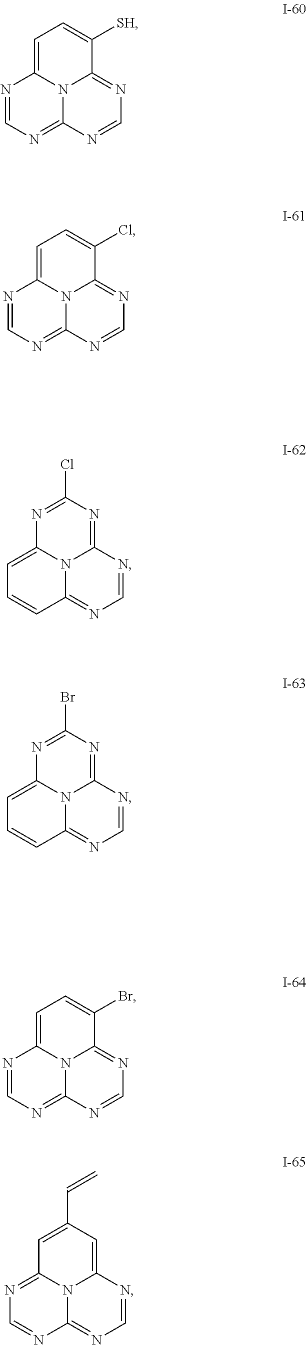

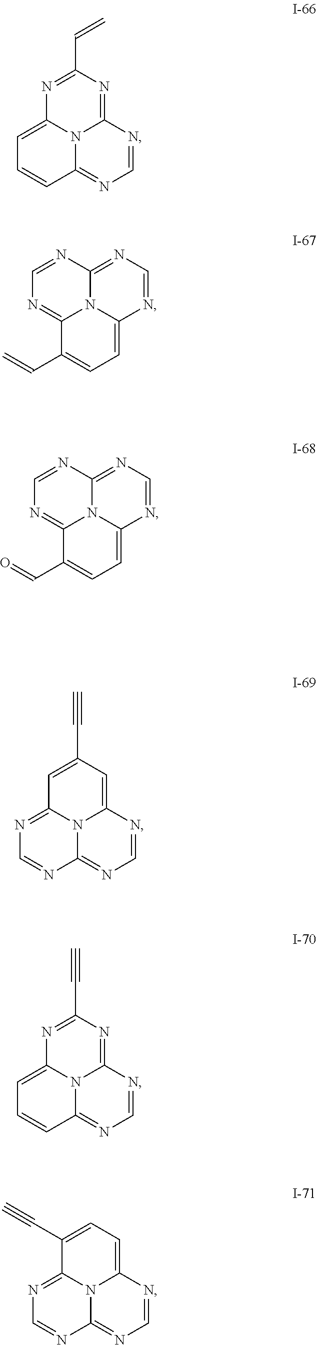

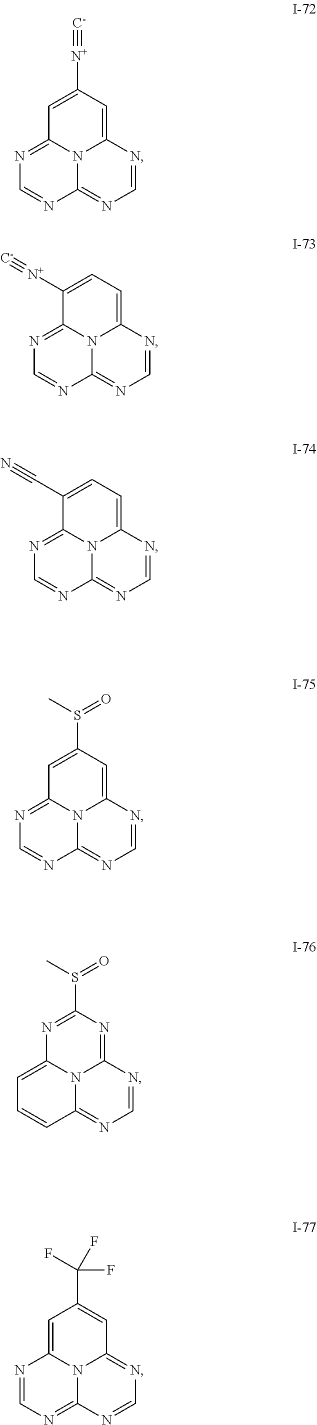 [SMILES: [C-]#[N+]C1=CC2=NC=NC3=NC=NC(=C1)N23 ; [C-]#[N+]C1=CC=C2N=CN=C3N=CN=C1N23 ; N#CC1=CC=C2N=CN=C3N=CN=C1N23 ; CS(=O)C1=CC2=NC=NC3=NC=NC(=C1)N23 ; CS(=O)C1=NC2=CC=CC3=NC=NC(=N1)N23 ; FC(F)(F)C1=CC2=NC=NC3=NC=NC(=C1)N23]

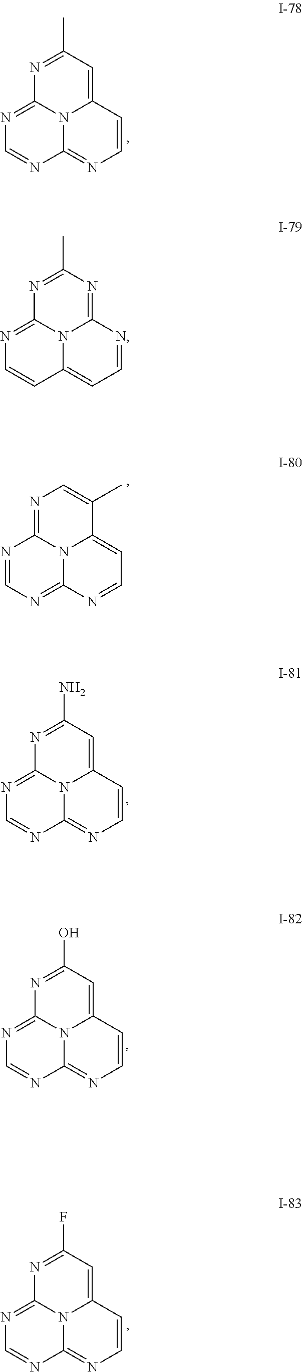

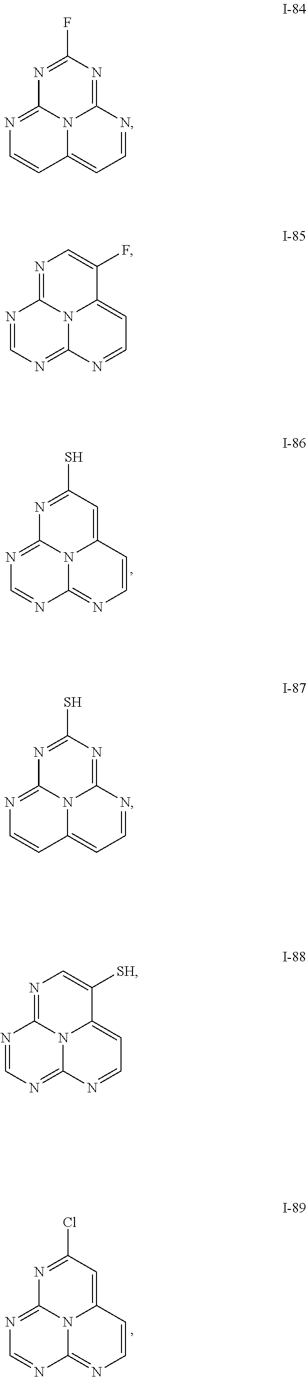 [SMILES: FC1=NC2=NC=CC3=CC=NC(=N1)N32 ; FC1=CN=C2N=CN=C3N=CC=C1N32 ; SC1=NC2=NC=NC3=NC=CC(=C1)N32 ; SC1=NC2=NC=CC3=CC=NC(=N1)N32 ; SC1=CN=C2N=CN=C3N=CC=C1N32 ; ClC1=NC2=NC=NC3=NC=CC(=C1)N32]

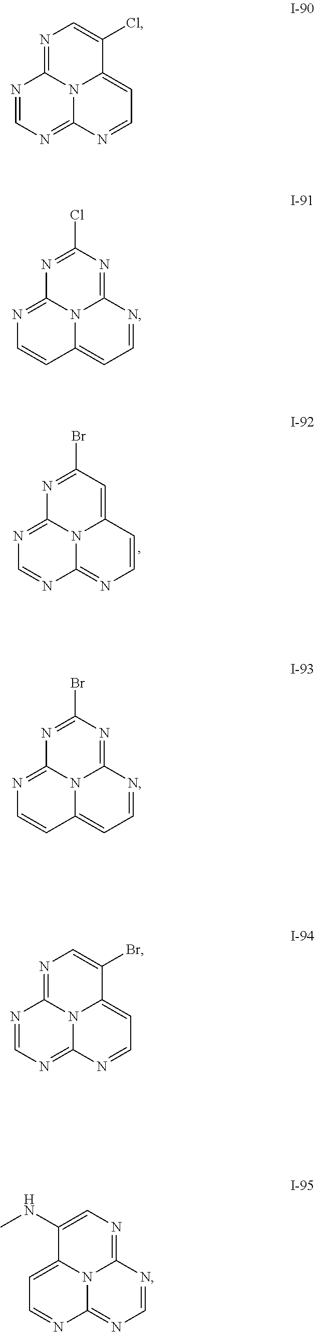

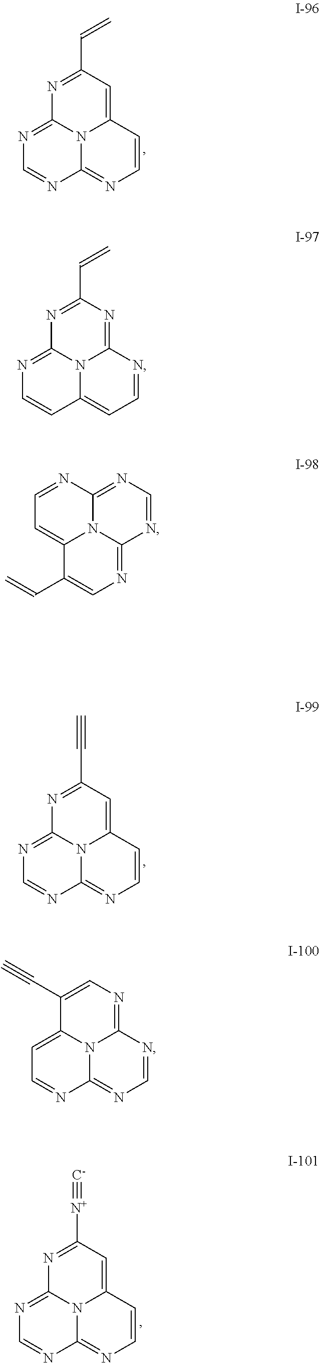

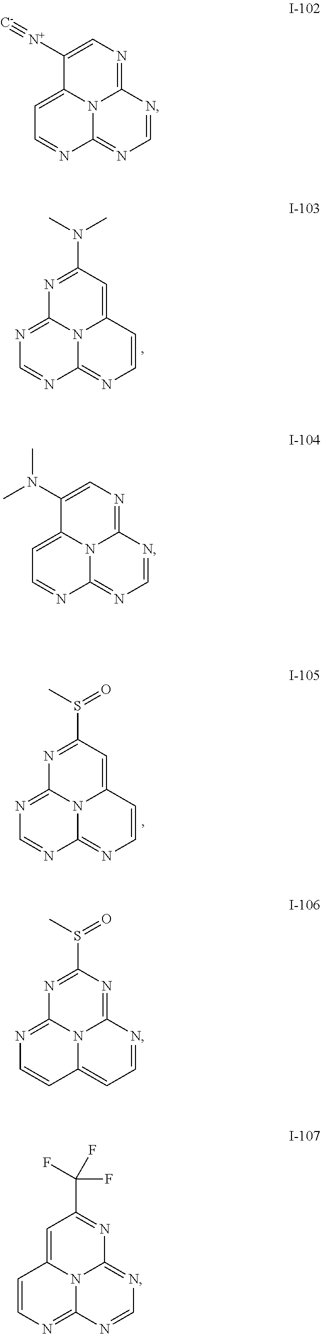 [SMILES: [C-]#[N+]C1=CN=C2N=CN=C3N=CC=C1N32 ; CN(C)C1=NC2=NC=NC3=NC=CC(=C1)N32 ; CN(C)C1=CN=C2N=CN=C3N=CC=C1N32 ; CS(=O)C1=NC2=NC=NC3=NC=CC(=C1)N32 ; CS(=O)C1=NC2=NC=CC3=CC=NC(=N1)N32 ; FC(F)(F)C1=NC2=NC=NC3=NC=CC(=C1)N32]

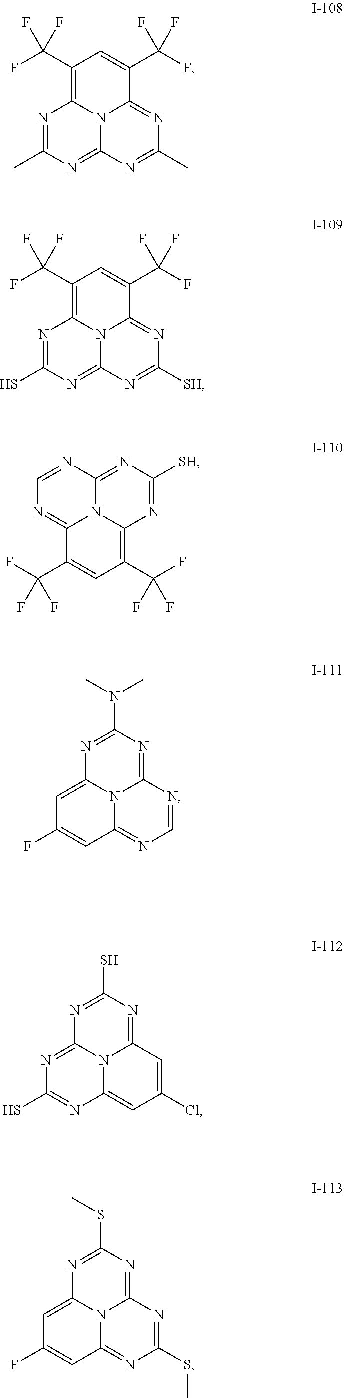 [SMILES: CC1=NC2=NC(C)=NC3=C(C(F)(F)F)C=C(C(F)(F)F)C(=N1)N23 ; FC(F)(F)C1=CC(C(F)(F)F)=C2N=C(S)N=C3N=C(S)N=C1N32 ; FC(F)(F)C1=CC(C(F)(F)F)=C2N=C(S)N=C3N=CN=C1N32 ; CN(C)C1=NC2=CC(F)=CC3=NC=NC(=N1)N23 ; SC1=NC2=CC(Cl)=CC3=NC(S)=NC(=N1)N23 ; CSC1=NC2=CC(F)=CC3=NC(SC)=NC(=N1)N23]

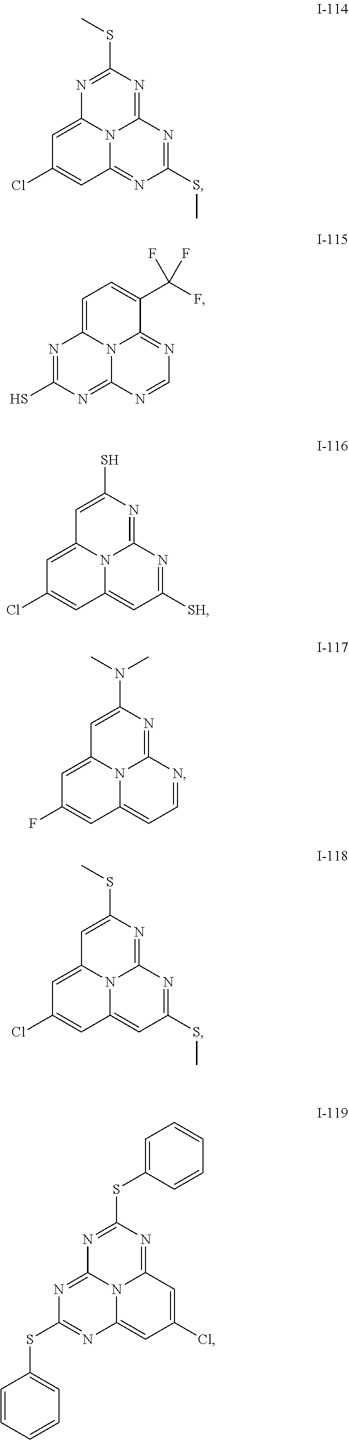

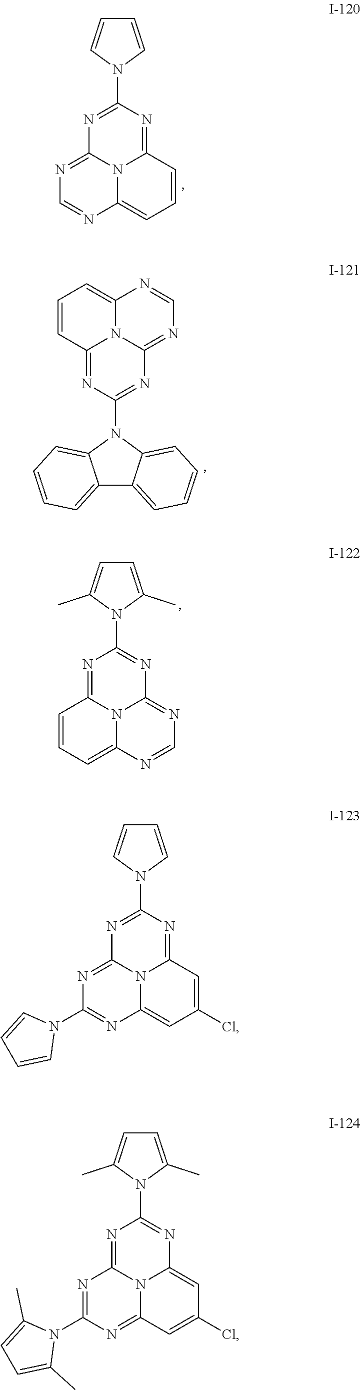

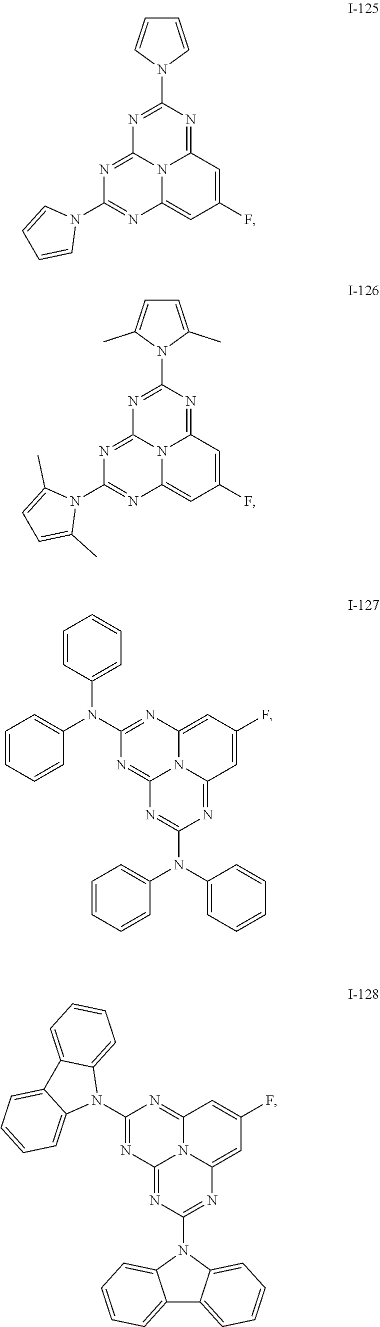 [SMILES: FC1=CC2=NC(n3cccc3)=NC3=NC(n4cccc4)=NC(=C1)N23 ; Cc1ccc(C)n1C1=NC2=CC(F)=CC3=NC(n4c(C)ccc4C)=NC(=N1)N23 ; FC1=CC2=NC(N(c3ccccc3)c3ccccc3)=NC3=NC(N(c4ccccc4)c4ccccc4)=NC(=C1)N23 ; FC1=CC2=NC(n3c4ccccc4c4ccccc43)=NC3=NC(n4c5ccccc5c5ccccc54)=NC(=C1)N23]

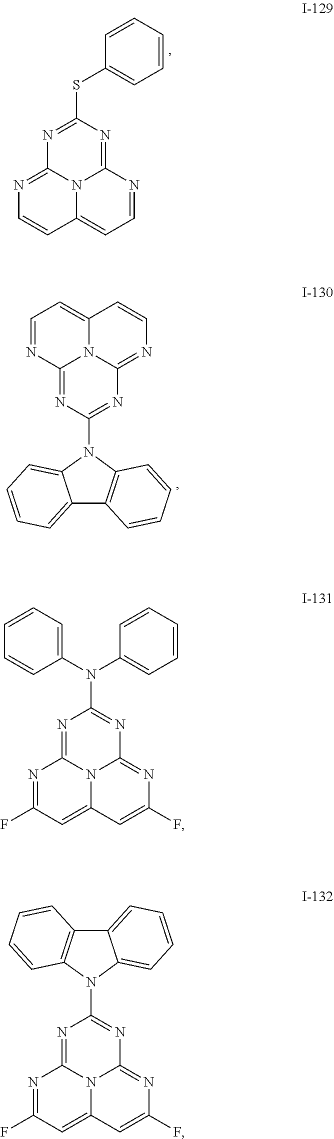 [SMILES: C1=CC2=CC=NC3=NC(Sc4ccccc4)=NC(=N1)N23 ; C1=CC2=CC=NC3=NC(n4c5ccccc5c5ccccc54)=NC(=N1)N23 ; FC1=CC2=CC(F)=NC3=NC(N(c4ccccc4)c4ccccc4)=NC(=N1)N23 ; FC1=CC2=CC(F)=NC3=NC(n4c5ccccc5c5ccccc54)=NC(=N1)N23]

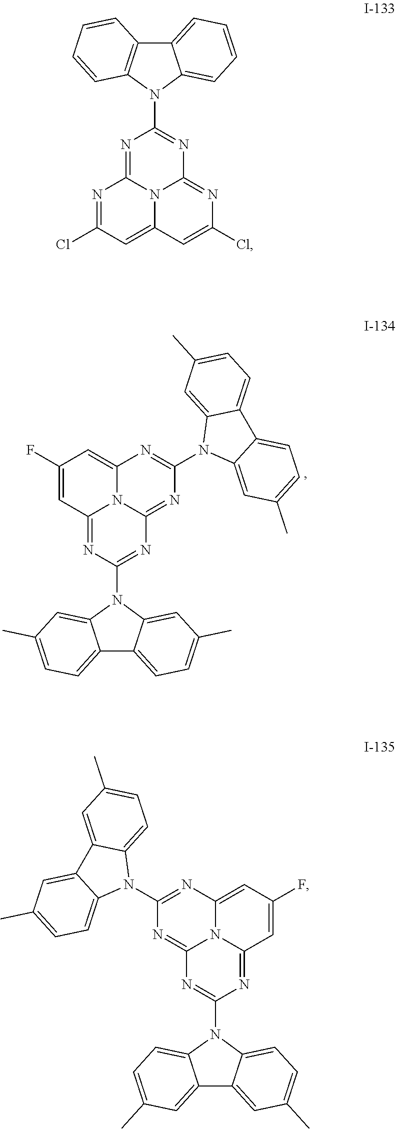

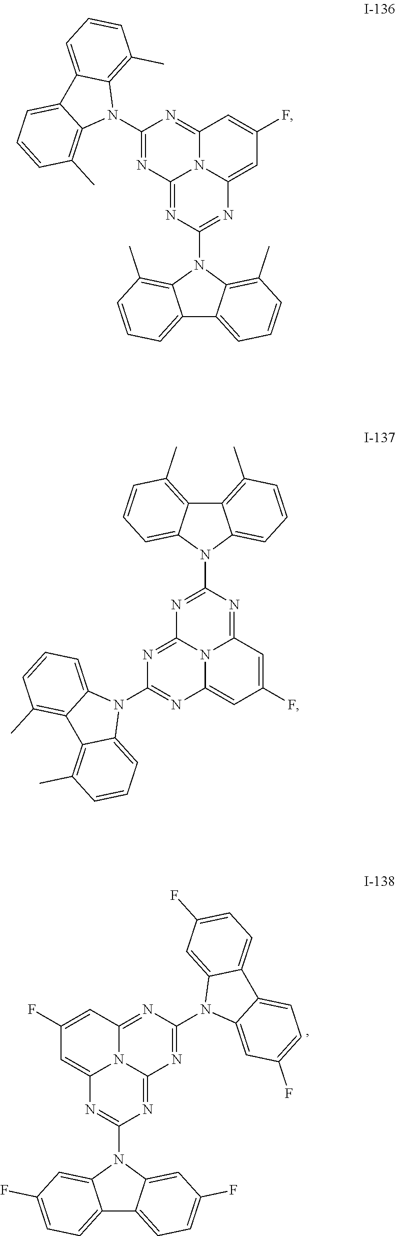 [SMILES: Cc1cccc2c3cccc(C)c3n(C3=NC4=CC(F)=CC5=NC(n6c7c(C)cccc7c7cccc(C)c76)=NC(=N3)N45)c12 ; Cc1cccc2c1c1c(C)cccc1n2C1=NC2=CC(F)=CC3=NC(n4c5cccc(C)c5c5c(C)cccc54)=NC(=N1)N23 ; FC1=CC2=NC(n3c4cc(F)ccc4c4ccc(F)cc43)=NC3=NC(n4c5cc(F)ccc5c5ccc(F)cc54)=NC(=C1)N23]

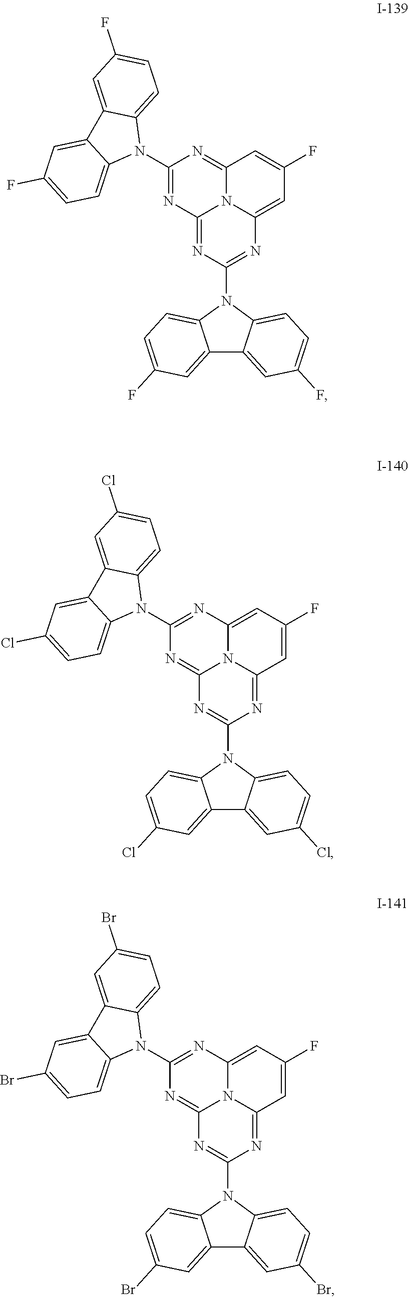 [SMILES: FC1=CC2=NC(n3c4ccc(F)cc4c4cc(F)ccc43)=NC3=NC(n4c5ccc(F)cc5c5cc(F)ccc54)=NC(=C1)N23 ; FC1=CC2=NC(n3c4ccc(Cl)cc4c4cc(Cl)ccc43)=NC3=NC(n4c5ccc(Cl)cc5c5cc(Cl)ccc54)=NC(=C1)N23 ; FC1=CC2=NC(n3c4ccc(Br)cc4c4cc(Br)ccc43)=NC3=NC(n4c5ccc(Br)cc5c5cc(Br)ccc54)=NC(=C1)N23]

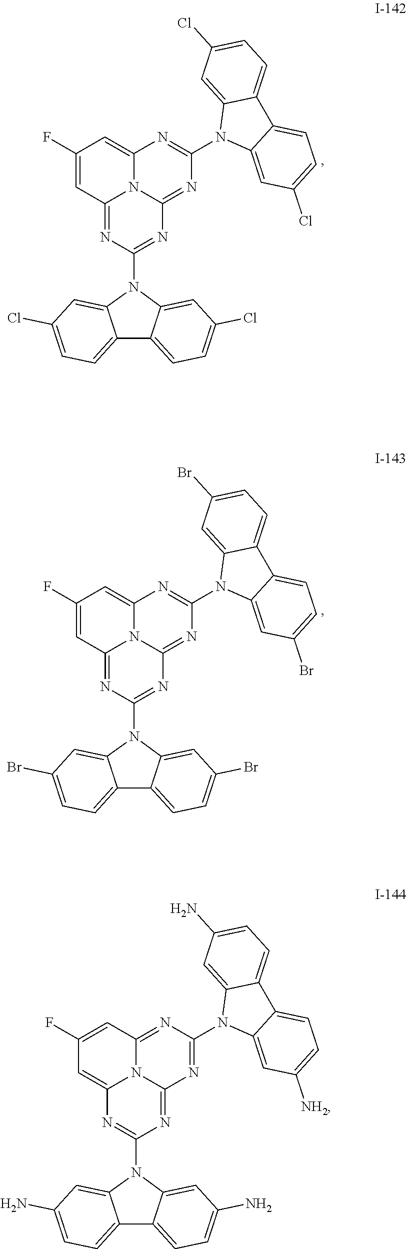 [SMILES: FC1=CC2=NC(n3c4cc(Cl)ccc4c4ccc(Cl)cc43)=NC3=NC(n4c5cc(Cl)ccc5c5ccc(Cl)cc54)=NC(=C1)N23 ; FC1=CC2=NC(n3c4cc(Br)ccc4c4ccc(Br)cc43)=NC3=NC(n4c5cc(Br)ccc5c5ccc(Br)cc54)=NC(=C1)N23 ; Nc1ccc2c3ccc(N)cc3n(C3=NC4=CC(F)=CC5=NC(n6c7cc(N)ccc7c7ccc(N)cc76)=NC(=N3)N45)c2c1]

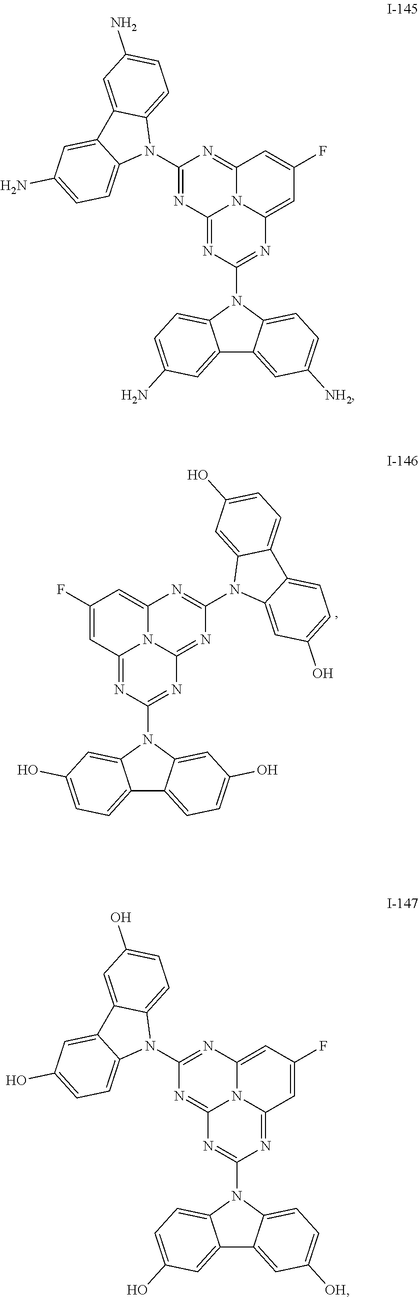 [SMILES: Nc1ccc2c(c1)c1cc(N)ccc1n2C1=NC2=CC(F)=CC3=NC(n4c5ccc(N)cc5c5cc(N)ccc54)=NC(=N1)N23 ; Oc1ccc2c3ccc(O)cc3n(C3=NC4=CC(F)=CC5=NC(n6c7cc(O)ccc7c7ccc(O)cc76)=NC(=N3)N45)c2c1 ; Oc1ccc2c(c1)c1cc(O)ccc1n2C1=NC2=CC(F)=CC3=NC(n4c5ccc(O)cc5c5cc(O)ccc54)=NC(=N1)N23]

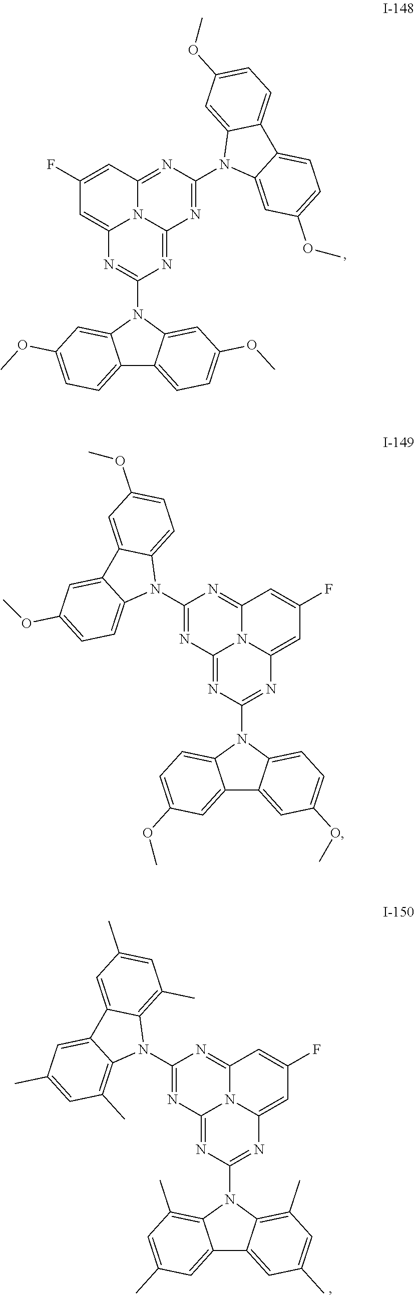

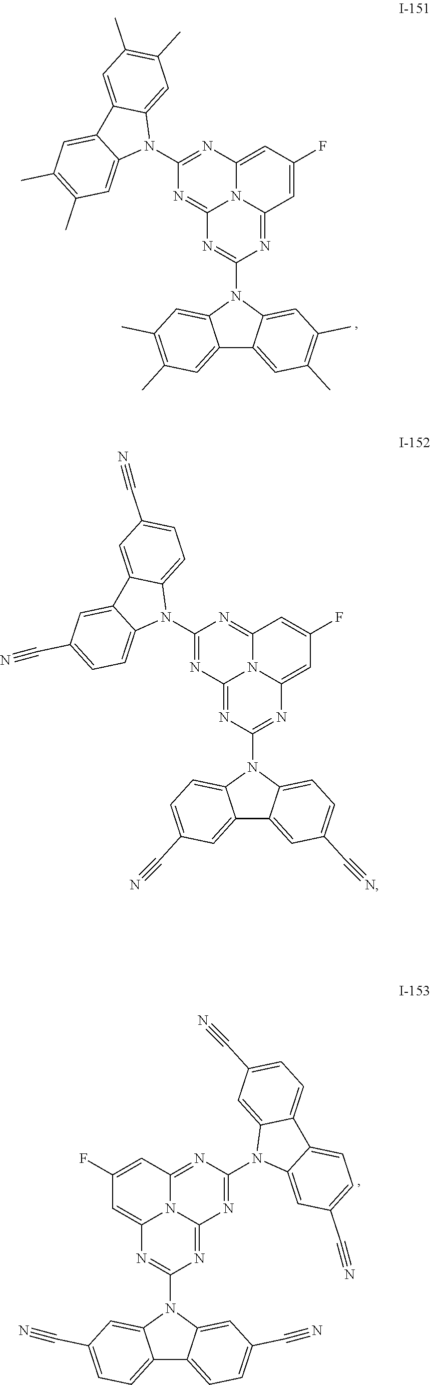

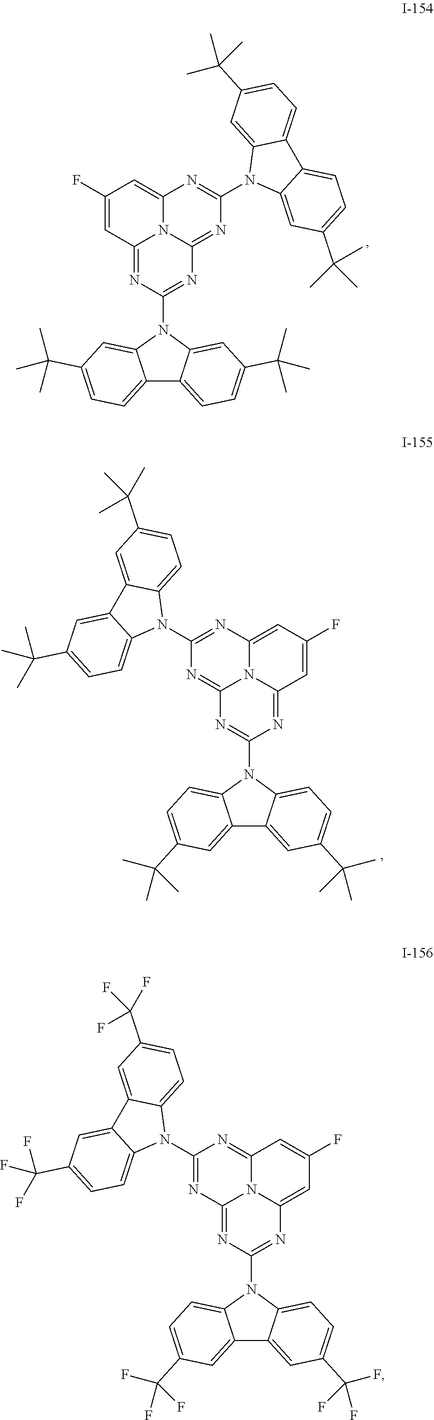

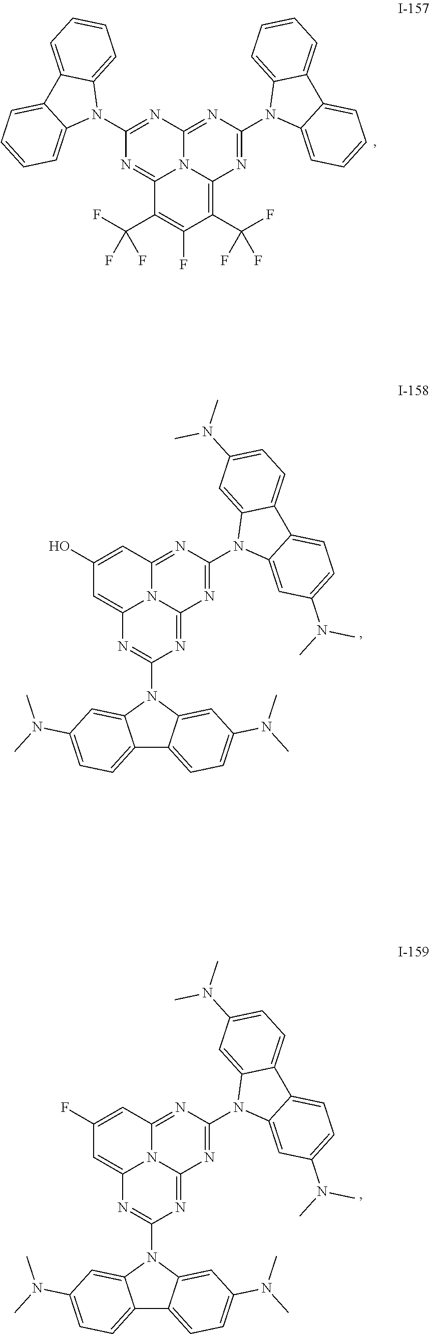

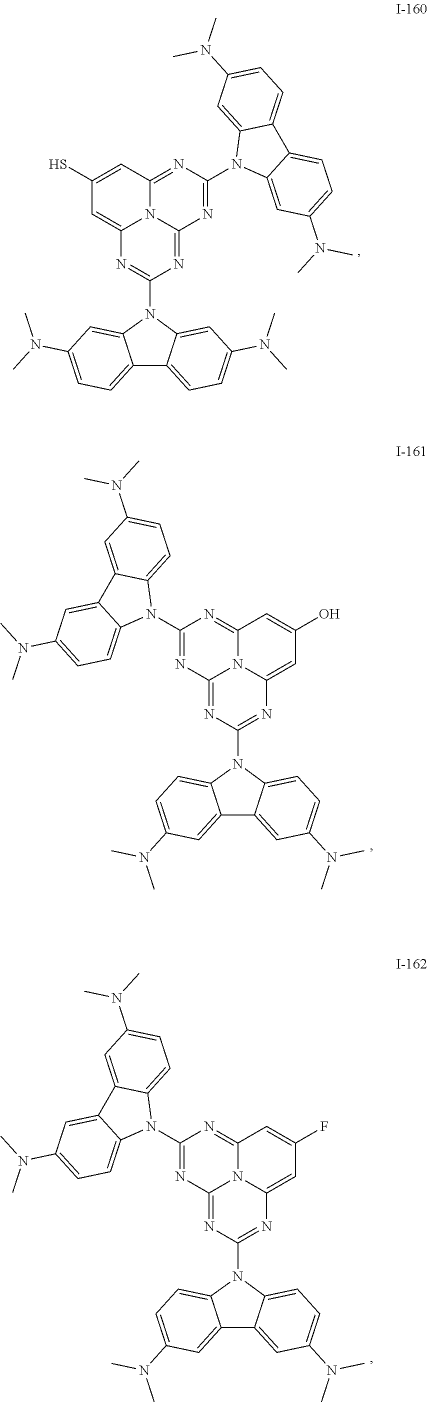

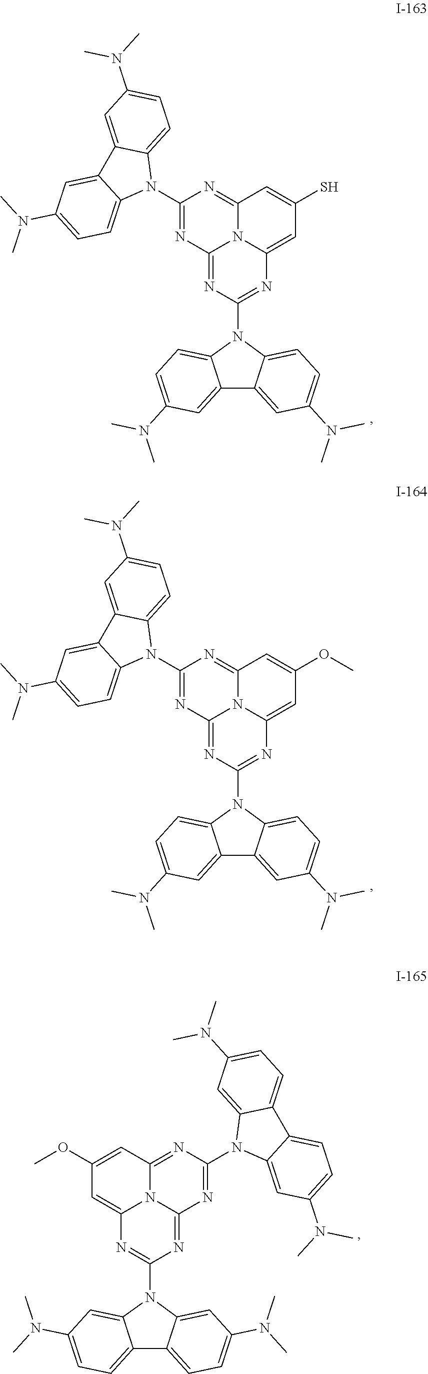

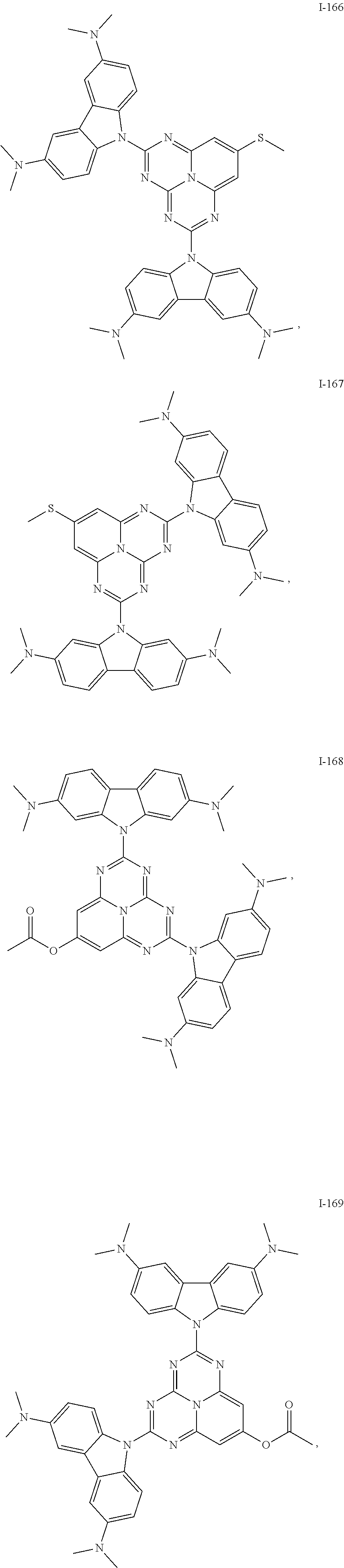

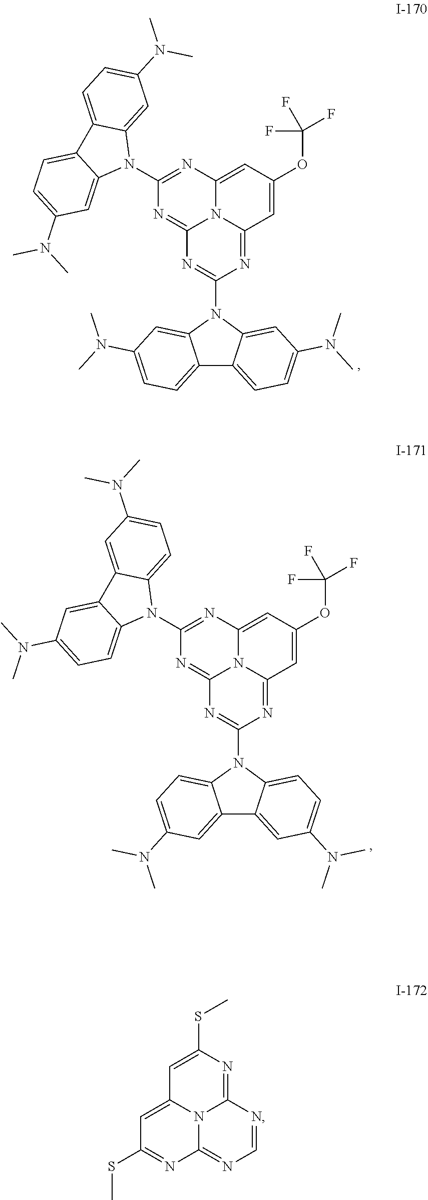

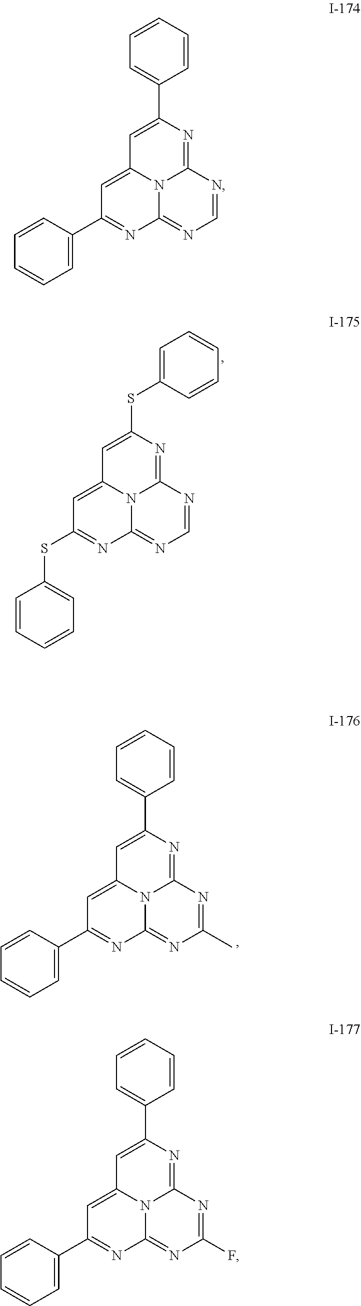 [SMILES: C1=NC2=NC(c3ccccc3)=CC3=CC(c4ccccc4)=NC(=N1)N32 ; C1=NC2=NC(Sc3ccccc3)=CC3=CC(Sc4ccccc4)=NC(=N1)N32 ; CC1=NC2=NC(c3ccccc3)=CC3=CC(c4ccccc4)=NC(=N1)N32 ; FC1=NC2=NC(c3ccccc3)=CC3=CC(c4ccccc4)=NC(=N1)N32]

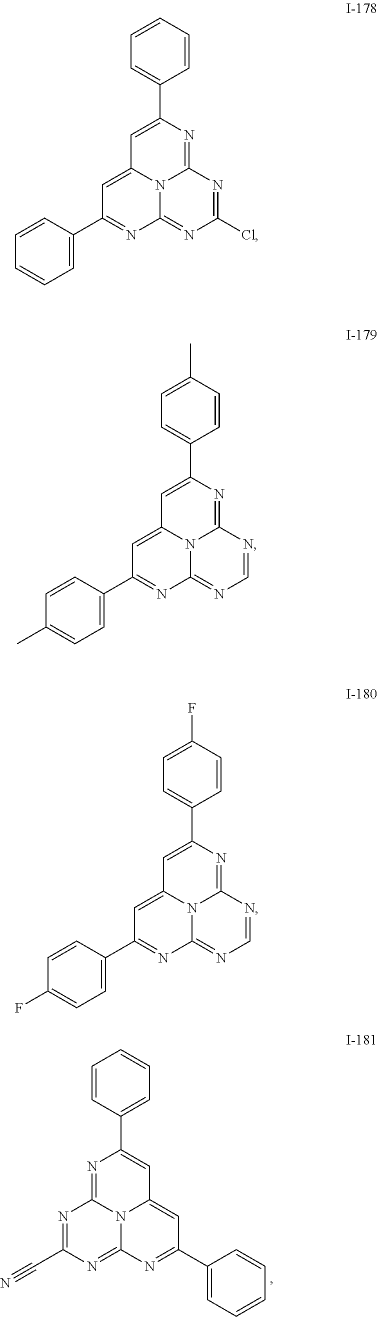 [SMILES: ClC1=NC2=NC(c3ccccc3)=CC3=CC(c4ccccc4)=NC(=N1)N32 ; Cc1ccc(C2=CC3=CC(c4ccc(C)cc4)=NC4=NC=NC(=N2)N34)cc1 ; Fc1ccc(C2=CC3=CC(c4ccc(F)cc4)=NC4=NC=NC(=N2)N34)cc1 ; N#CC1=NC2=NC(c3ccccc3)=CC3=CC(c4ccccc4)=NC(=N1)N32]

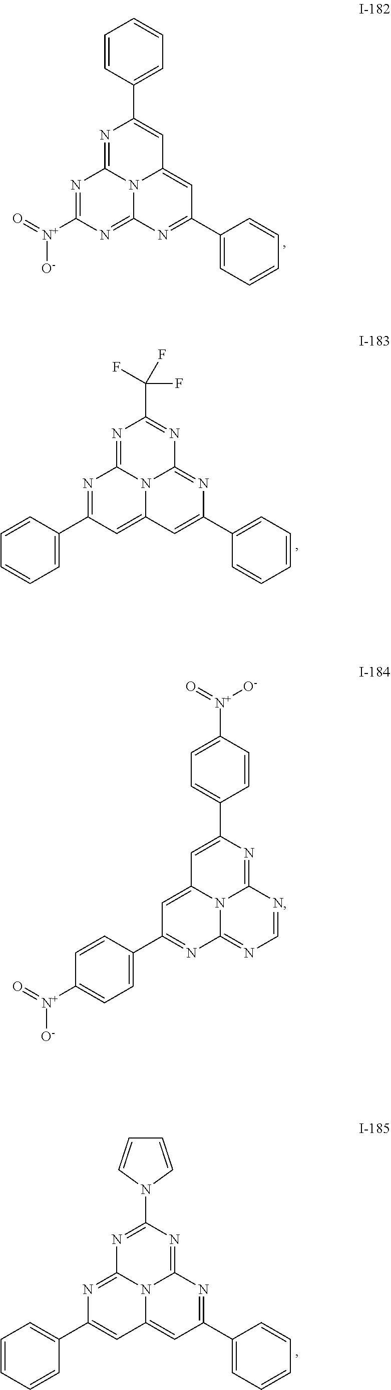 [SMILES: O=[N+]([O-])C1=NC2=NC(c3ccccc3)=CC3=CC(c4ccccc4)=NC(=N1)N32 ; FC(F)(F)C1=NC2=NC(c3ccccc3)=CC3=CC(c4ccccc4)=NC(=N1)N32 ; O=[N+]([O-])c1ccc(C2=CC3=CC(c4ccc([N+](=O)[O-])cc4)=NC4=NC=NC(=N2)N34)cc1 ; C1=C(c2ccccc2)N=C2N=C(n3cccc3)N=C3N=C(c4ccccc4)C=C1N23]

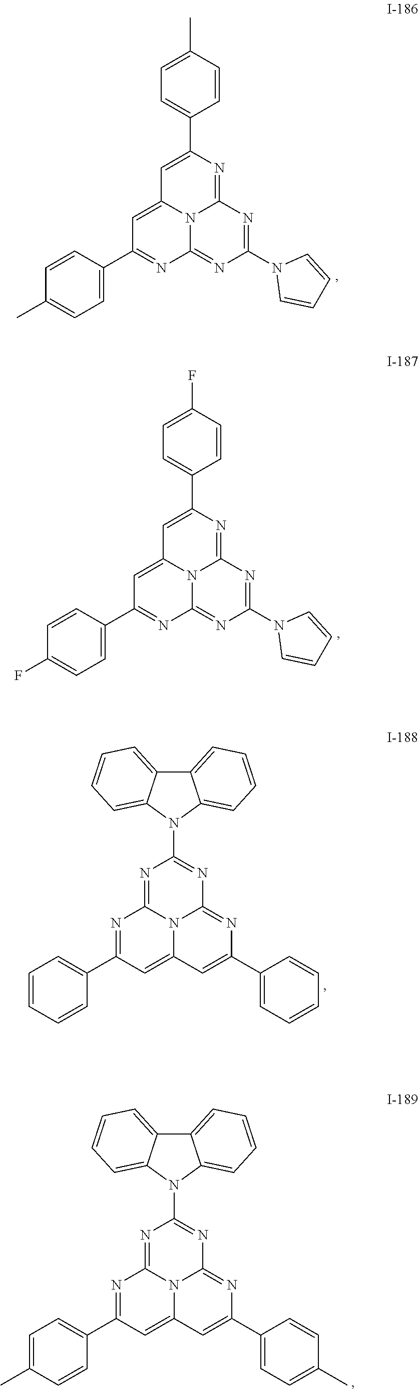

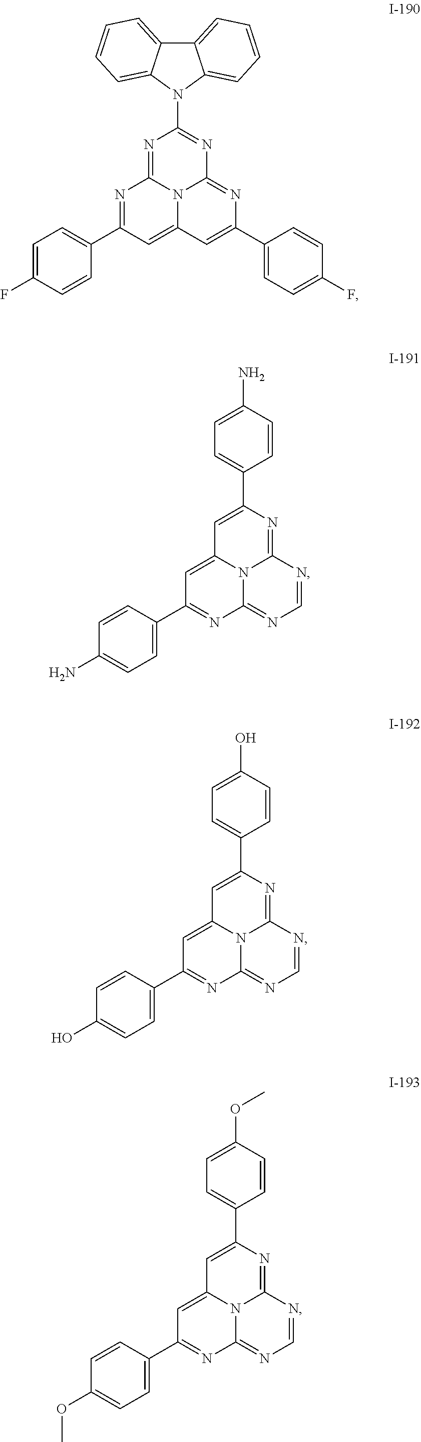 [SMILES: Fc1ccc(C2=CC3=CC(c4ccc(F)cc4)=NC4=NC(n5c6ccccc6c6ccccc65)=NC(=N2)N34)cc1 ; Nc1ccc(C2=CC3=CC(c4ccc(N)cc4)=NC4=NC=NC(=N2)N34)cc1 ; Oc1ccc(C2=CC3=CC(c4ccc(O)cc4)=NC4=NC=NC(=N2)N34)cc1 ; COc1ccc(C2=CC3=CC(c4ccc(OC)cc4)=NC4=NC=NC(=N2)N34)cc1]

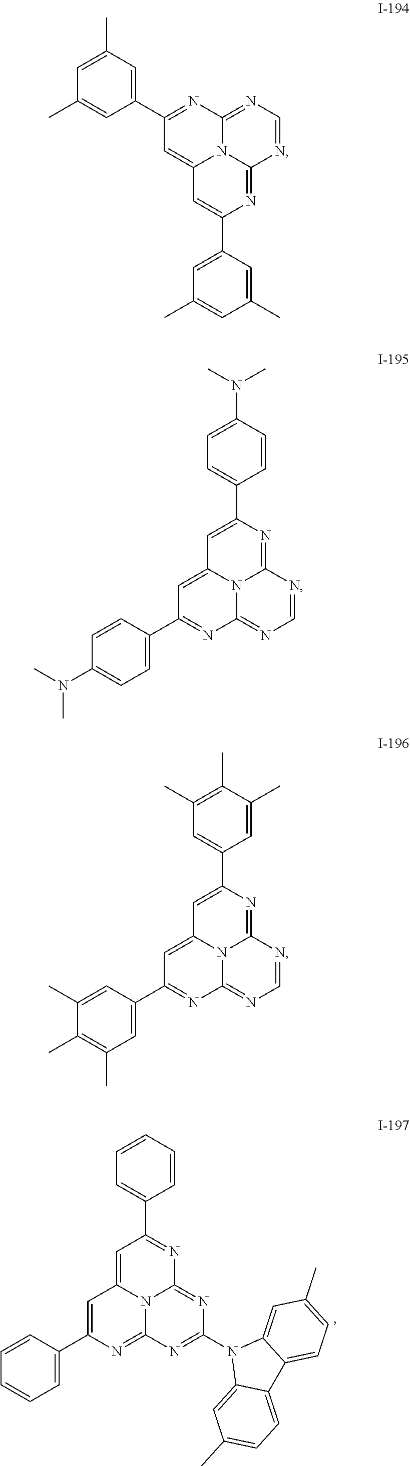

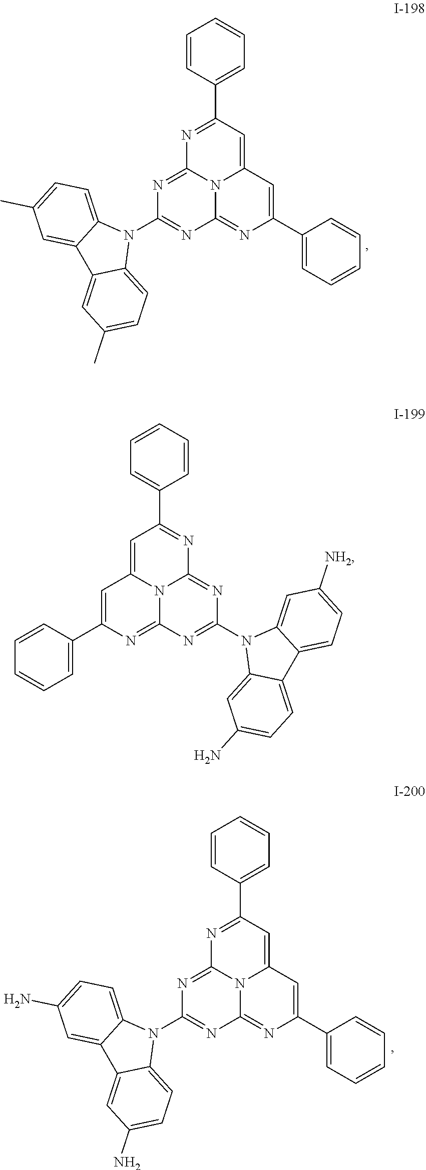 [SMILES: Cc1ccc2c(c1)c1cc(C)ccc1n2C1=NC2=NC(c3ccccc3)=CC3=CC(c4ccccc4)=NC(=N1)N32 ; Nc1ccc2c3ccc(N)cc3n(C3=NC4=NC(c5ccccc5)=CC5=CC(c6ccccc6)=NC(=N3)N54)c2c1 ; Nc1ccc2c(c1)c1cc(N)ccc1n2C1=NC2=NC(c3ccccc3)=CC3=CC(c4ccccc4)=NC(=N1)N32]

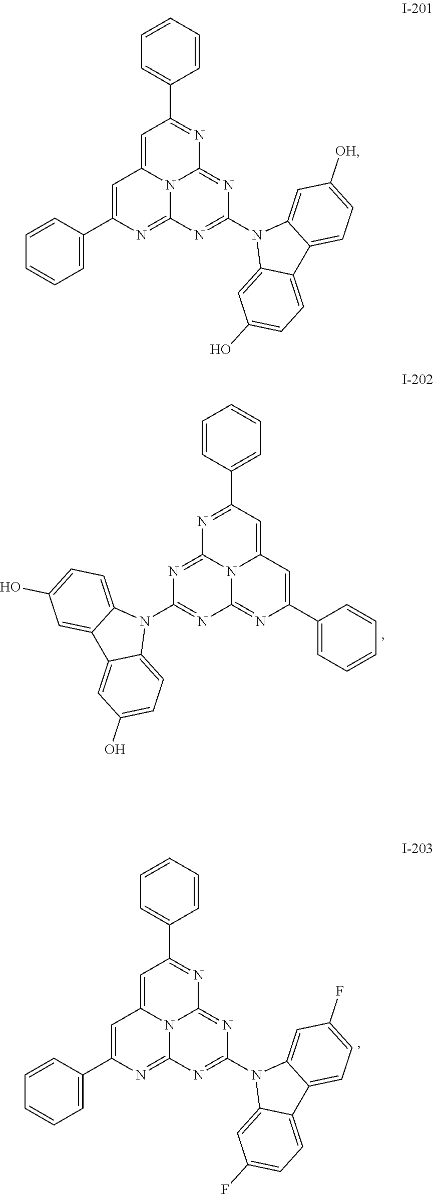 [SMILES: Oc1ccc2c3ccc(O)cc3n(C3=NC4=NC(c5ccccc5)=CC5=CC(c6ccccc6)=NC(=N3)N54)c2c1 ; Oc1ccc2c(c1)c1cc(O)ccc1n2C1=NC2=NC(c3ccccc3)=CC3=CC(c4ccccc4)=NC(=N1)N32 ; Fc1ccc2c3ccc(F)cc3n(C3=NC4=NC(c5ccccc5)=CC5=CC(c6ccccc6)=NC(=N3)N54)c2c1]

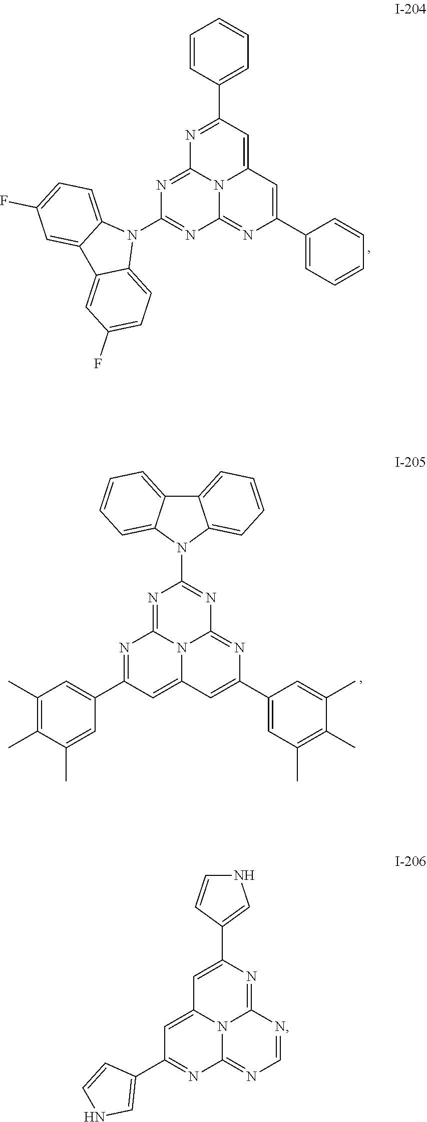

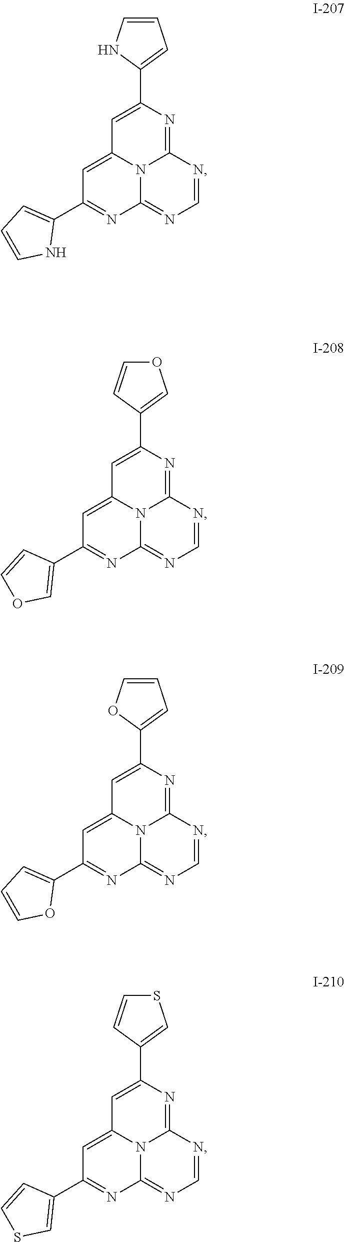 [SMILES: C1=NC2=NC(c3ccc[nH]3)=CC3=CC(c4ccc[nH]4)=NC(=N1)N32 ; C1=NC2=NC(c3ccoc3)=CC3=CC(c4ccoc4)=NC(=N1)N32 ; C1=NC2=NC(c3ccco3)=CC3=CC(c4ccco4)=NC(=N1)N32 ; C1=NC2=NC(c3ccsc3)=CC3=CC(c4ccsc4)=NC(=N1)N32]

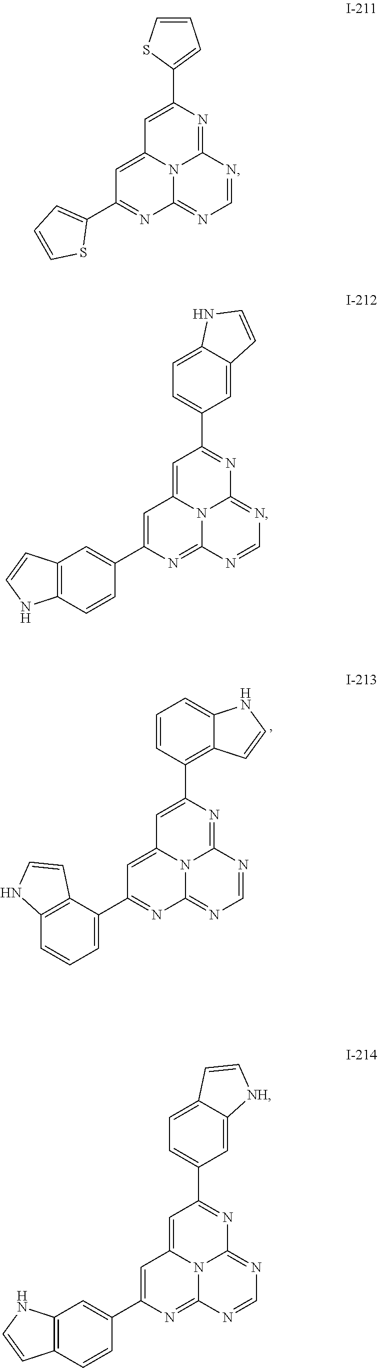 [SMILES: C1=NC2=NC(c3cccs3)=CC3=CC(c4cccs4)=NC(=N1)N32 ; C1=NC2=NC(c3ccc4[nH]ccc4c3)=CC3=CC(c4ccc5[nH]ccc5c4)=NC(=N1)N32 ; C1=NC2=NC(c3cccc4[nH]ccc34)=CC3=CC(c4cccc5[nH]ccc45)=NC(=N1)N32 ; C1=NC2=NC(c3ccc4cc[nH]c4c3)=CC3=CC(c4ccc5cc[nH]c5c4)=NC(=N1)N32]

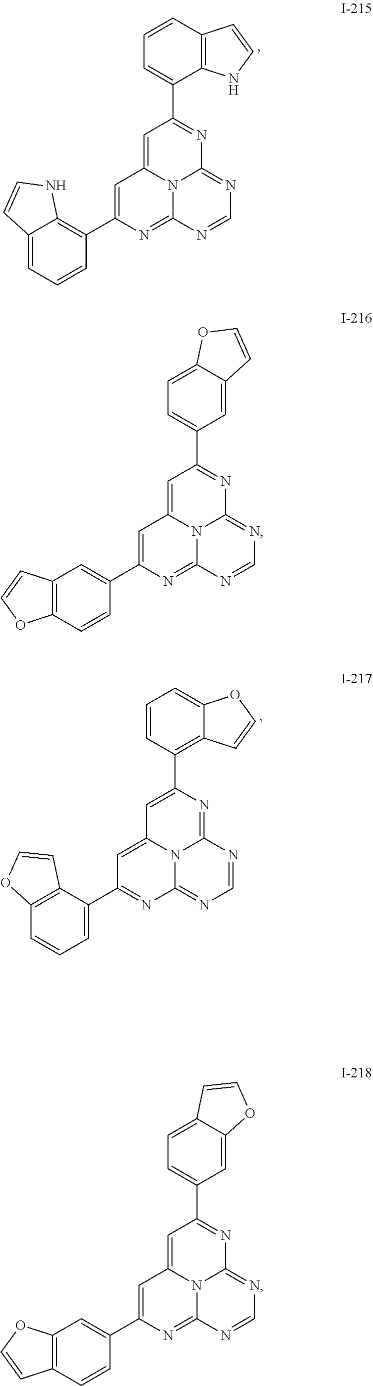 [SMILES: C1=NC2=NC(c3cccc4cc[nH]c34)=CC3=CC(c4cccc5cc[nH]c45)=NC(=N1)N32 ; C1=NC2=NC(c3ccc4occc4c3)=CC3=CC(c4ccc5occc5c4)=NC(=N1)N32 ; C1=NC2=NC(c3cccc4occc34)=CC3=CC(c4cccc5occc45)=NC(=N1)N32 ; C1=NC2=NC(c3ccc4ccoc4c3)=CC3=CC(c4ccc5ccoc5c4)=NC(=N1)N32]

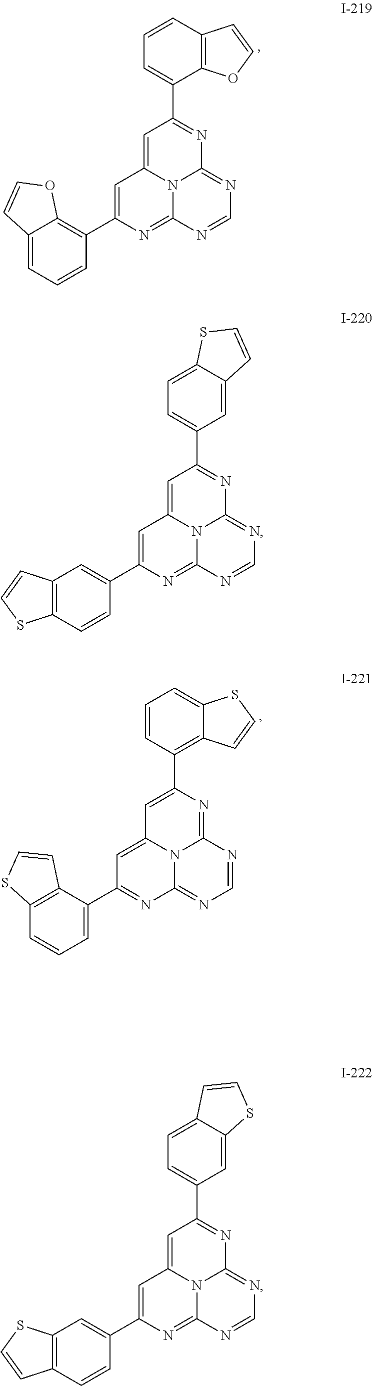 [SMILES: C1=NC2=NC(c3cccc4ccoc34)=CC3=CC(c4cccc5ccoc45)=NC(=N1)N32 ; C1=NC2=NC(c3ccc4sccc4c3)=CC3=CC(c4ccc5sccc5c4)=NC(=N1)N32 ; C1=NC2=NC(c3cccc4sccc34)=CC3=CC(c4cccc5sccc45)=NC(=N1)N32 ; C1=NC2=NC(c3ccc4ccsc4c3)=CC3=CC(c4ccc5ccsc5c4)=NC(=N1)N32]

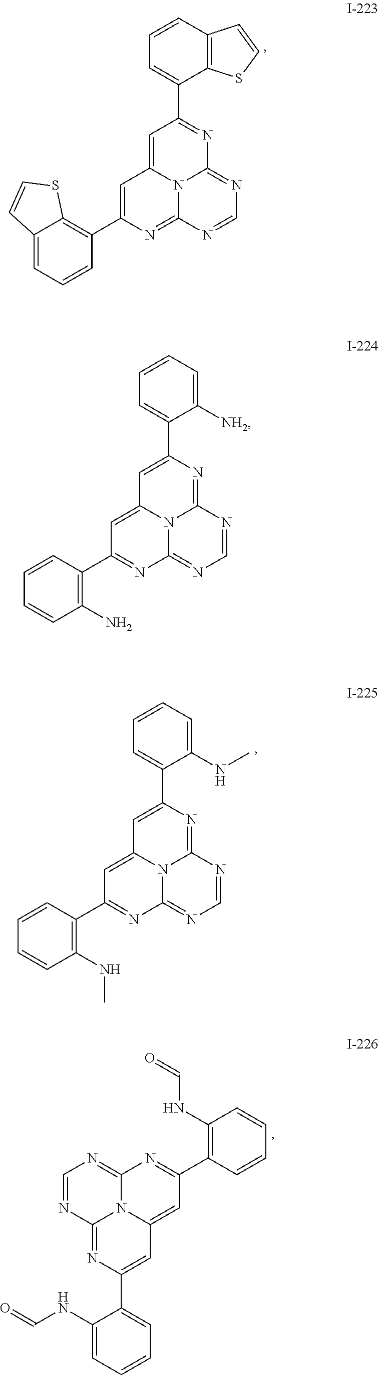 [SMILES: C1=NC2=NC(c3cccc4ccsc34)=CC3=CC(c4cccc5ccsc45)=NC(=N1)N32 ; Nc1ccccc1C1=CC2=CC(c3ccccc3N)=NC3=NC=NC(=N1)N23 ; CNc1ccccc1C1=CC2=CC(c3ccccc3NC)=NC3=NC=NC(=N1)N23 ; O=CNc1ccccc1C1=CC2=CC(c3ccccc3NC=O)=NC3=NC=NC(=N1)N23]

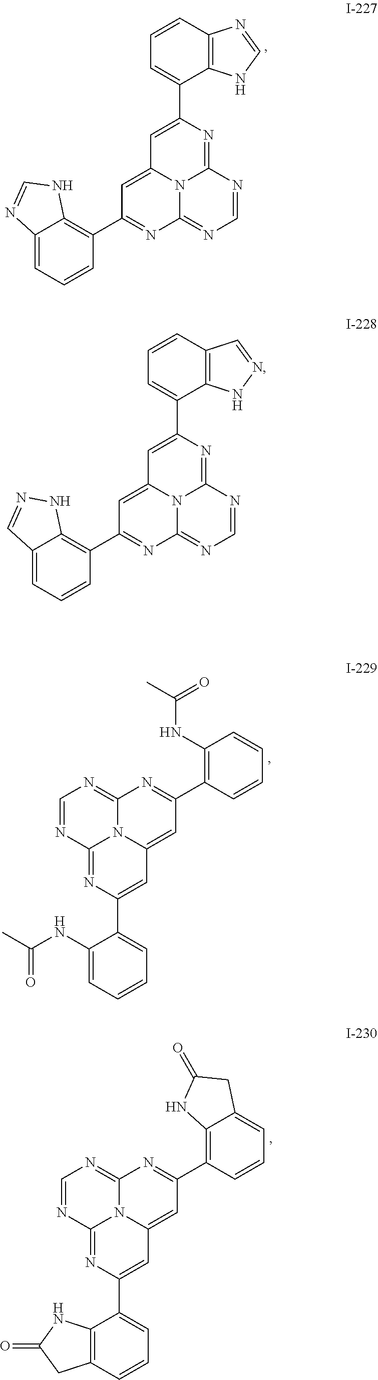 [SMILES: C1=NC2=NC(c3cccc4nc[nH]c34)=CC3=CC(c4cccc5nc[nH]c45)=NC(=N1)N32 ; C1=NC2=NC(c3cccc4cn[nH]c34)=CC3=CC(c4cccc5cn[nH]c45)=NC(=N1)N32 ; CC(=O)Nc1ccccc1C1=CC2=CC(c3ccccc3NC(C)=O)=NC3=NC=NC(=N1)N23 ; O=C1Cc2cccc(C3=CC4=CC(c5cccc6c5NC(=O)C6)=NC5=NC=NC(=N3)N45)c2N1]

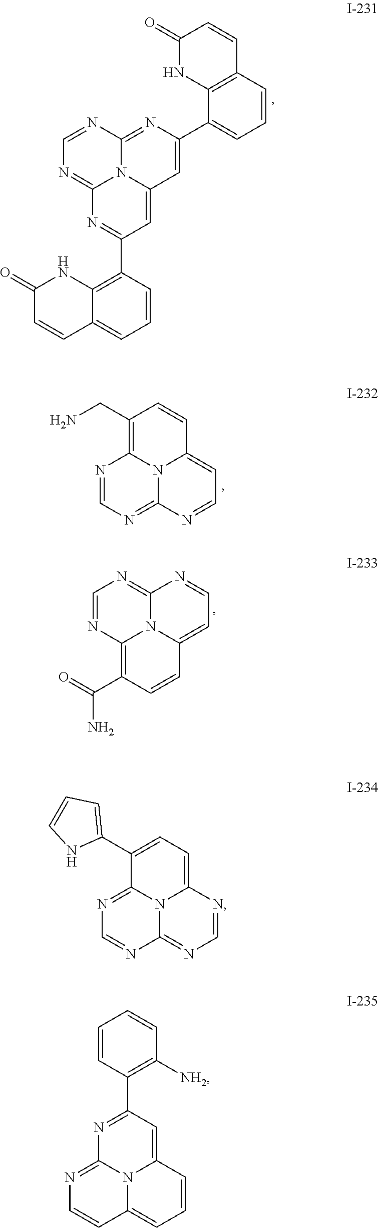 [SMILES: O=c1ccc2cccc(C3=CC4=CC(c5cccc6ccc(=O)[nH]c56)=NC5=NC=NC(=N3)N45)c2[nH]1 ; NCC1=C2N=CN=C3N=CC=C(C=C1)N32 ; NC(=O)C1=C2N=CN=C3N=CC=C(C=C1)N32 ; C1=NC2=CC=C(c3ccc[nH]3)C3=NC=NC(=N1)N23 ; Nc1ccccc1C1=NC2=NC=CC3=CC=CC(=C1)N32]

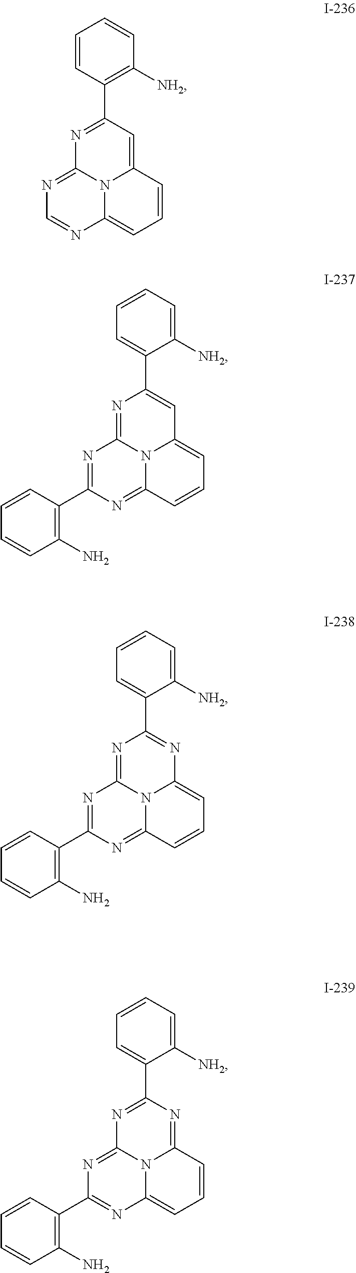 [SMILES: Nc1ccccc1C1=NC2=NC=NC3=CC=CC(=C1)N32 ; Nc1ccccc1C1=CC2=CC=CC3=NC(c4ccccc4N)=NC(=N1)N23 ; Nc1ccccc1C1=NC2=CC=CC3=NC(c4ccccc4N)=NC(=N1)N23 ; Nc1ccccc1C1=NC2=CC=CC3=NC(c4ccccc4N)=NC(=N1)N23]

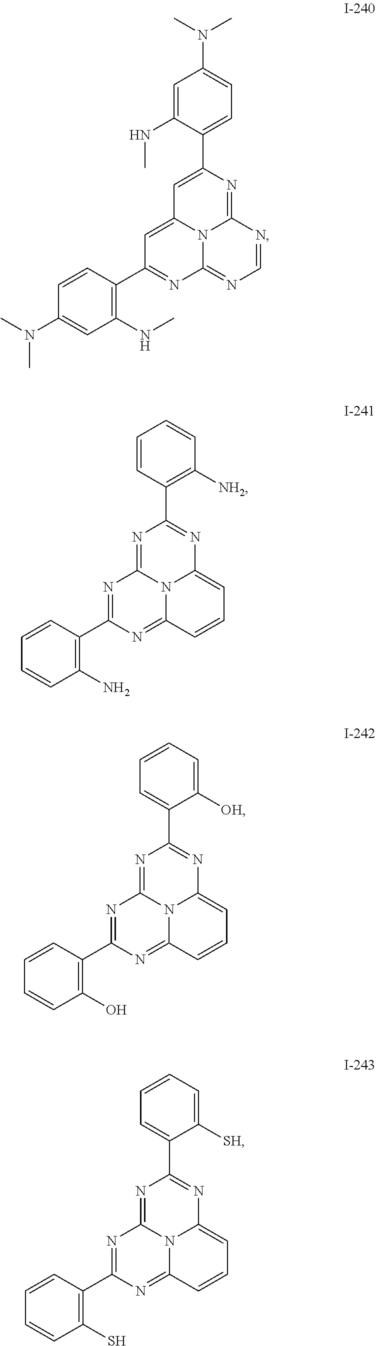 [SMILES: CNc1cc(N(C)C)ccc1C1=CC2=CC(c3ccc(N(C)C)cc3NC)=NC3=NC=NC(=N1)N23 ; Nc1ccccc1C1=NC2=CC=CC3=NC(c4ccccc4N)=NC(=N1)N23 ; Oc1ccccc1C1=NC2=CC=CC3=NC(c4ccccc4O)=NC(=N1)N23 ; Sc1ccccc1C1=NC2=CC=CC3=NC(c4ccccc4S)=NC(=N1)N23]

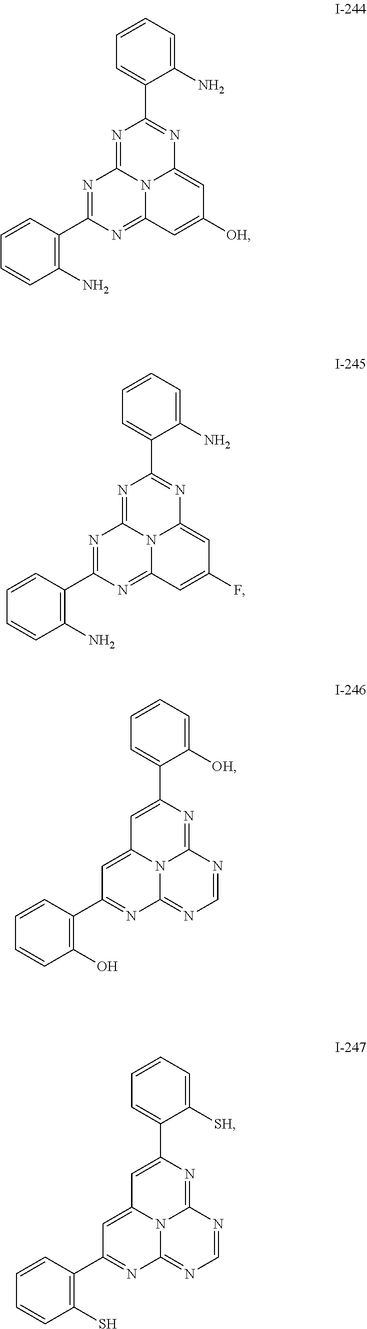 [SMILES: Nc1ccccc1C1=NC2=CC(O)=CC3=NC(c4ccccc4N)=NC(=N1)N23 ; Nc1ccccc1C1=NC2=CC(F)=CC3=NC(c4ccccc4N)=NC(=N1)N23 ; Oc1ccccc1C1=CC2=CC(c3ccccc3O)=NC3=NC=NC(=N1)N23 ; Sc1ccccc1C1=CC2=CC(c3ccccc3S)=NC3=NC=NC(=N1)N23]

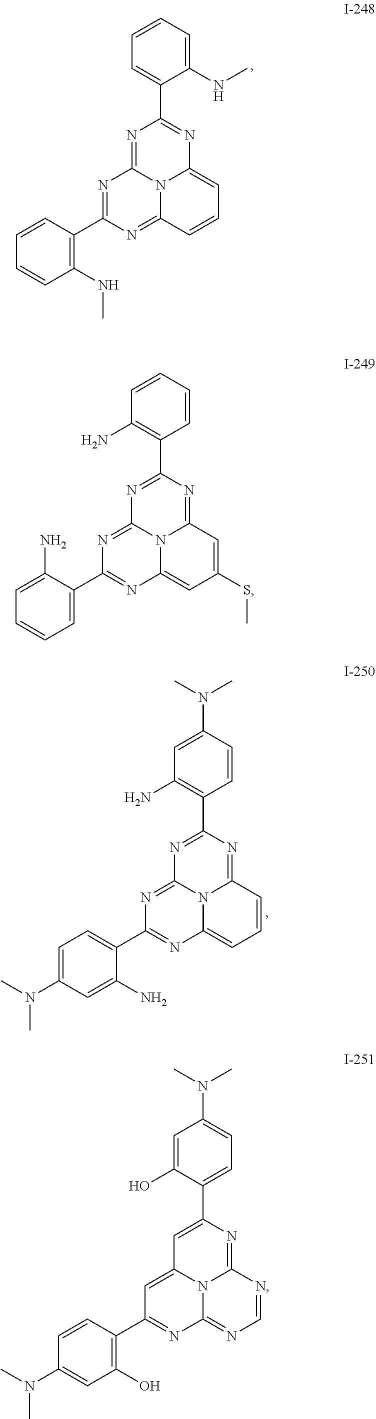

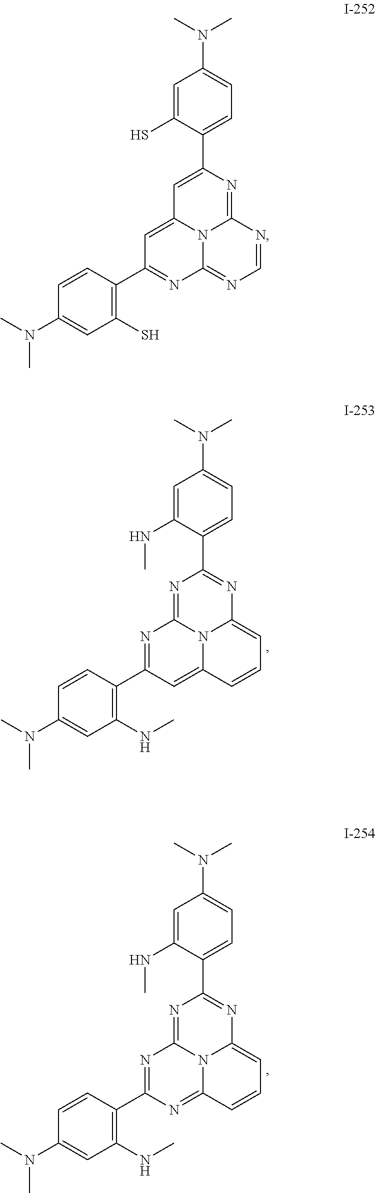 [SMILES: CN(C)c1ccc(C2=CC3=CC(c4ccc(N(C)C)cc4S)=NC4=NC=NC(=N2)N34)c(S)c1 ; CNc1cc(N(C)C)ccc1C1=NC2=NC(c3ccc(N(C)C)cc3NC)=NC3=CC=CC(=C1)N32 ; CNc1cc(N(C)C)ccc1C1=NC2=CC=CC3=NC(c4ccc(N(C)C)cc4NC)=NC(=N1)N23]

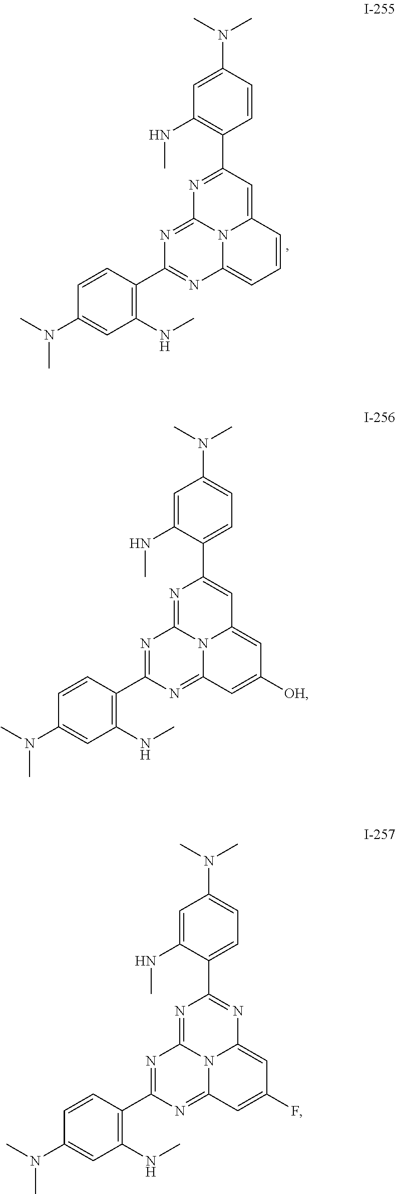 [SMILES: CNc1cc(N(C)C)ccc1C1=NC2=NC(c3ccc(N(C)C)cc3NC)=NC3=CC=CC(=C1)N32 ; CNc1cc(N(C)C)ccc1C1=CC2=CC(O)=CC3=NC(c4ccc(N(C)C)cc4NC)=NC(=N1)N23 ; CNc1cc(N(C)C)ccc1C1=NC2=CC(F)=CC3=NC(c4ccc(N(C)C)cc4NC)=NC(=N1)N23]

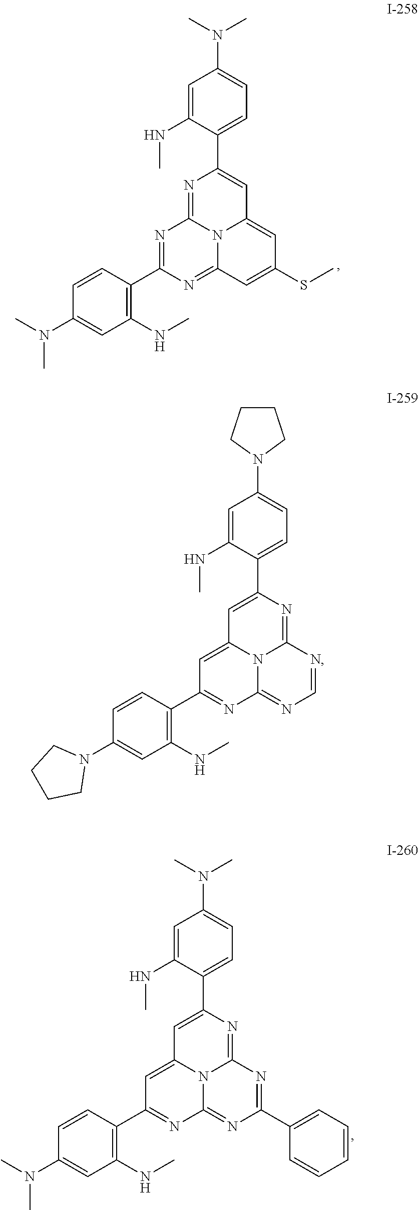

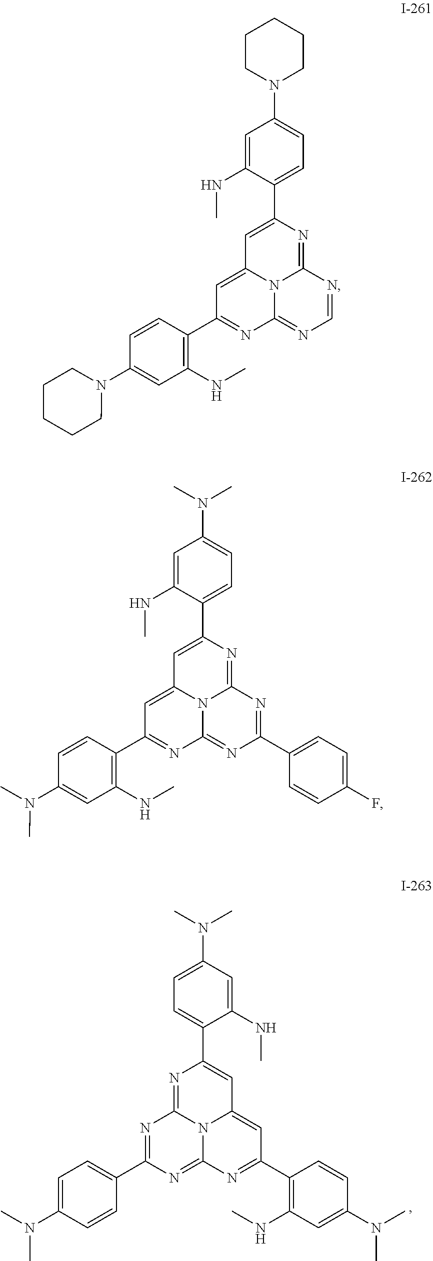

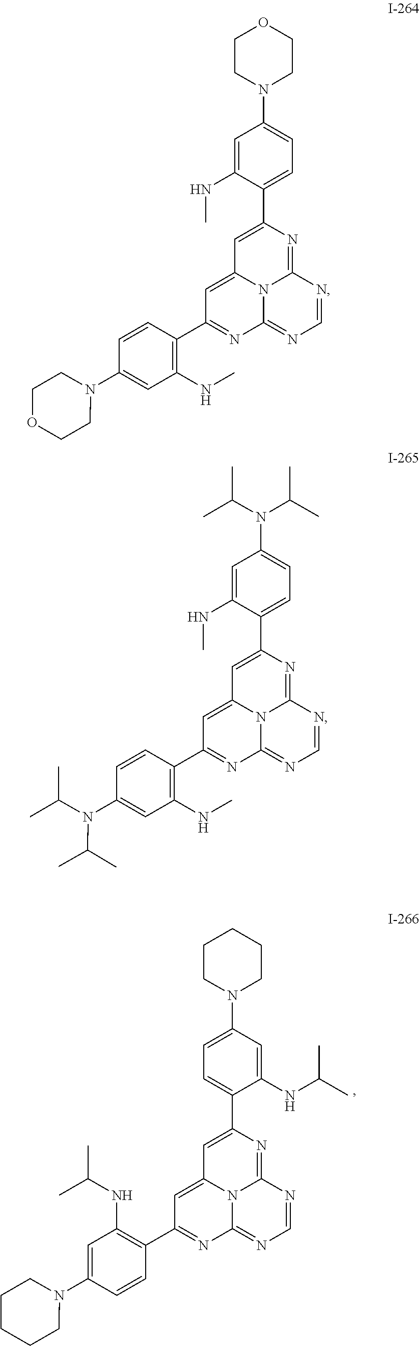 [SMILES: CNc1cc(N2CCOCC2)ccc1C1=CC2=CC(c3ccc(N4CCOCC4)cc3NC)=NC3=NC=NC(=N1)N23 ; CNc1cc(N(C(C)C)C(C)C)ccc1C1=CC2=CC(c3ccc(N(C(C)C)C(C)C)cc3NC)=NC3=NC=NC(=N1)N23 ; CC(C)Nc1cc(N2CCCCC2)ccc1C1=CC2=CC(c3ccc(N4CCCCC4)cc3NC(C)C)=NC3=NC=NC(=N1)N23]

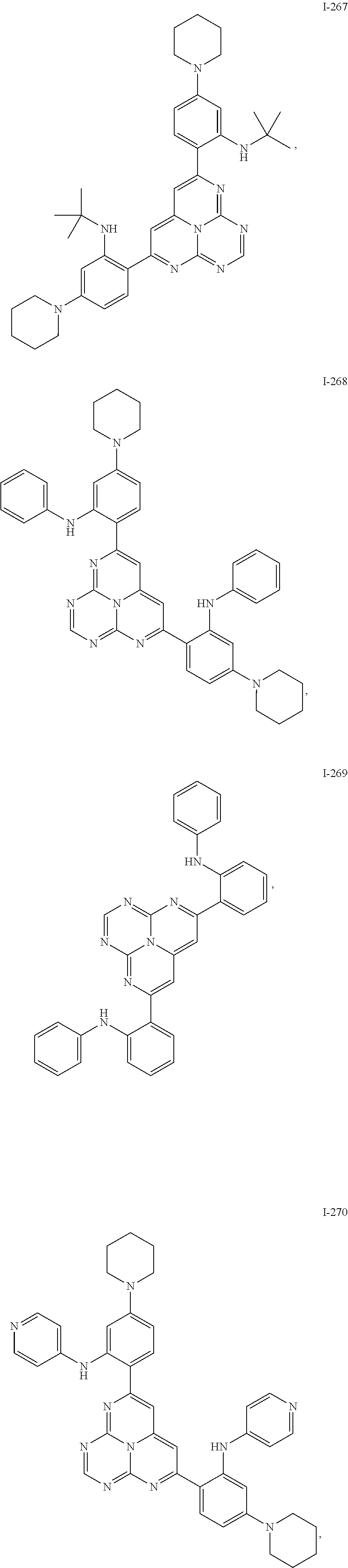 [SMILES: CC(C)(C)Nc1cc(N2CCCCC2)ccc1C1=CC2=CC(c3ccc(N4CCCCC4)cc3NC(C)(C)C)=NC3=NC=NC(=N1)N23 ; C1=NC2=NC(c3ccc(N4CCCCC4)cc3Nc3ccccc3)=CC3=CC(c4ccc(N5CCCCC5)cc4Nc4ccccc4)=NC(=N1)N32 ; C1=NC2=NC(c3ccccc3Nc3ccccc3)=CC3=CC(c4ccccc4Nc4ccccc4)=NC(=N1)N32 ; C1=NC2=NC(c3ccc(N4CCCCC4)cc3Nc3ccncc3)=CC3=CC(c4ccc(N5CCCCC5)cc4Nc4ccncc4)=NC(=N1)N32]

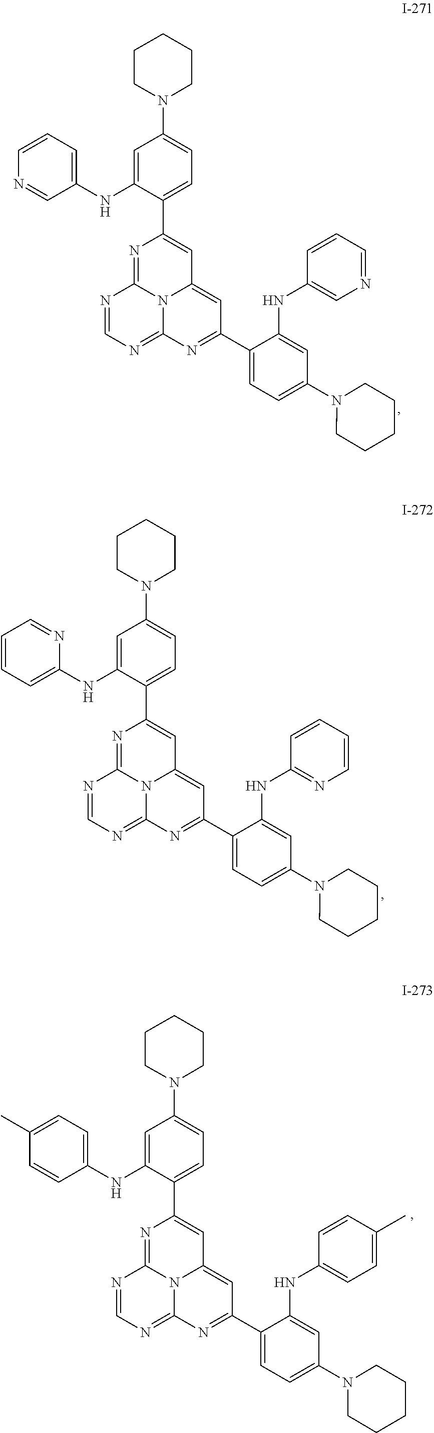

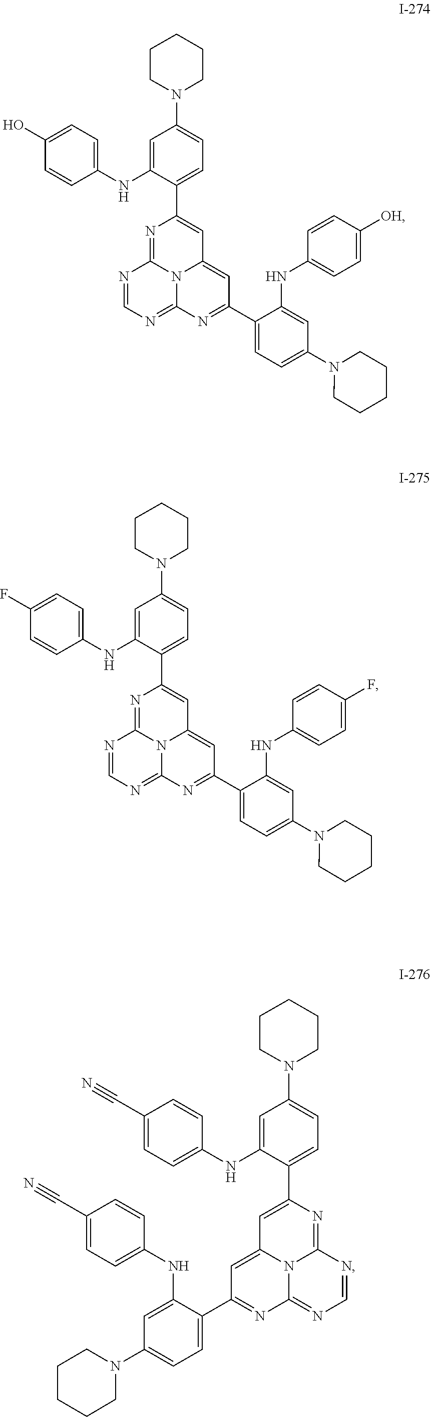 [SMILES: Oc1ccc(Nc2cc(N3CCCCC3)ccc2C2=CC3=CC(c4ccc(N5CCCCC5)cc4Nc4ccc(O)cc4)=NC4=NC=NC(=N2)N34)cc1 ; Fc1ccc(Nc2cc(N3CCCCC3)ccc2C2=CC3=CC(c4ccc(N5CCCCC5)cc4Nc4ccc(F)cc4)=NC4=NC=NC(=N2)N34)cc1 ; N#Cc1ccc(Nc2cc(N3CCCCC3)ccc2C2=CC3=CC(c4ccc(N5CCCCC5)cc4Nc4ccc(C#N)cc4)=NC4=NC=NC(=N2)N34)cc1]

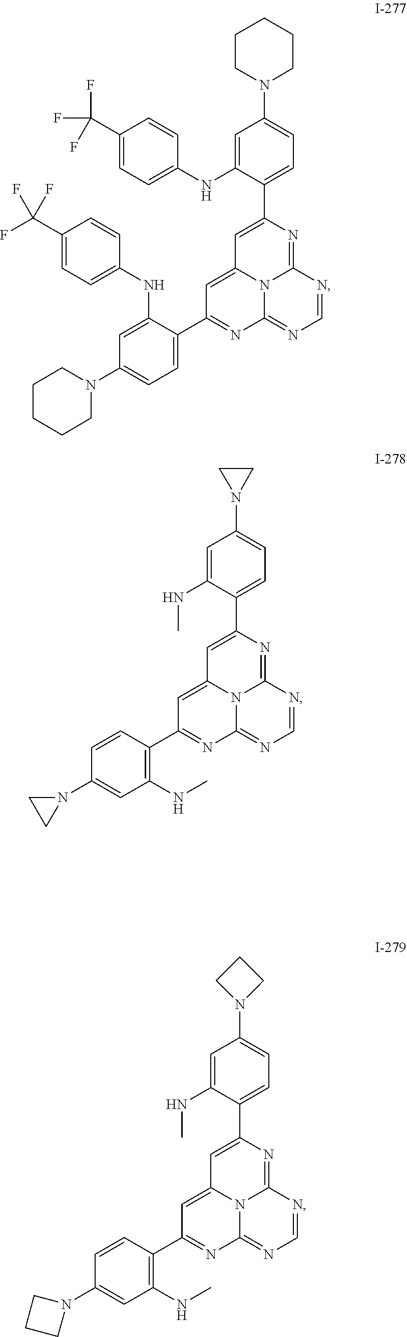 [SMILES: FC(F)(F)c1ccc(Nc2cc(N3CCCCC3)ccc2C2=CC3=CC(c4ccc(N5CCCCC5)cc4Nc4ccc(C(F)(F)F)cc4)=NC4=NC=NC(=N2)N34)cc1 ; CNc1cc(N2CC2)ccc1C1=CC2=CC(c3ccc(N4CC4)cc3NC)=NC3=NC=NC(=N1)N23 ; CNc1cc(N2CCC2)ccc1C1=CC2=CC(c3ccc(N4CCC4)cc3NC)=NC3=NC=NC(=N1)N23]

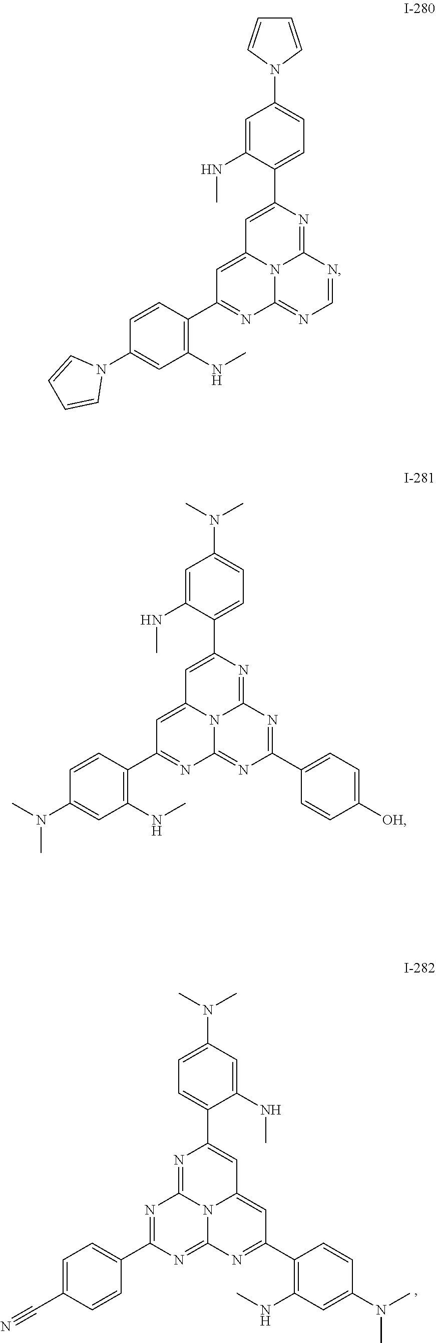 [SMILES: CNc1cc(-n2cccc2)ccc1C1=CC2=CC(c3ccc(-n4cccc4)cc3NC)=NC3=NC=NC(=N1)N23 ; CNc1cc(N(C)C)ccc1C1=CC2=CC(c3ccc(N(C)C)cc3NC)=NC3=NC(c4ccc(O)cc4)=NC(=N1)N23 ; CNc1cc(N(C)C)ccc1C1=CC2=CC(c3ccc(N(C)C)cc3NC)=NC3=NC(c4ccc(C#N)cc4)=NC(=N1)N23]

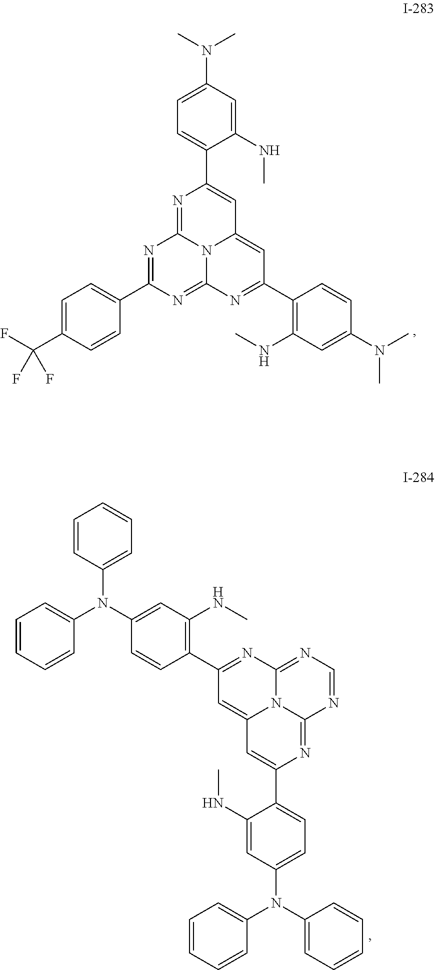 [SMILES: CNc1cc(N(C)C)ccc1C1=CC2=CC(c3ccc(N(C)C)cc3NC)=NC3=NC(c4ccc(C(F)(F)F)cc4)=NC(=N1)N23 ; CNc1cc(N(c2ccccc2)c2ccccc2)ccc1C1=CC2=CC(c3ccc(N(c4ccccc4)c4ccccc4)cc3NC)=NC3=NC=NC(=N1)N23]

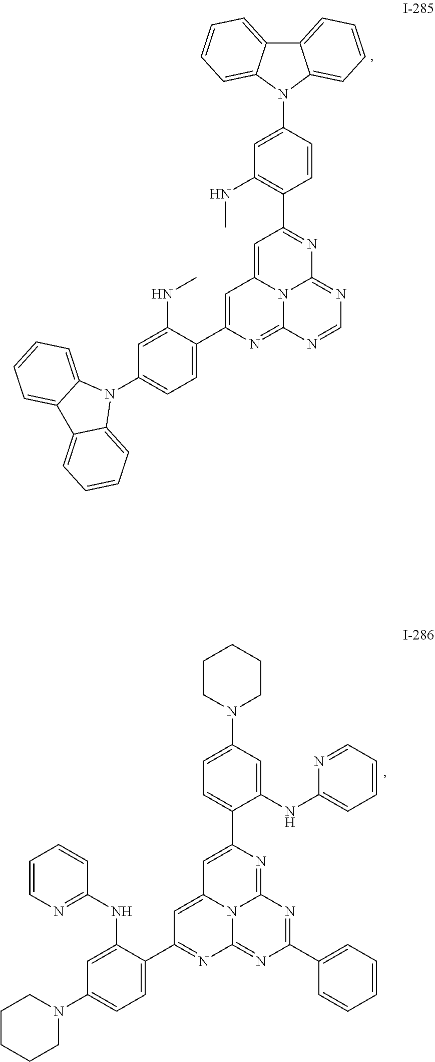 [SMILES: CNc1cc(-n2c3ccccc3c3ccccc32)ccc1C1=CC2=CC(c3ccc(-n4c5ccccc5c5ccccc54)cc3NC)=NC3=NC=NC(=N1)N23 ; C1=C(c2ccc(N3CCCCC3)cc2Nc2ccccn2)N=C2N=C(c3ccccc3)N=C3N=C(c4ccc(N5CCCCC5)cc4Nc4ccccn4)C=C1N23]

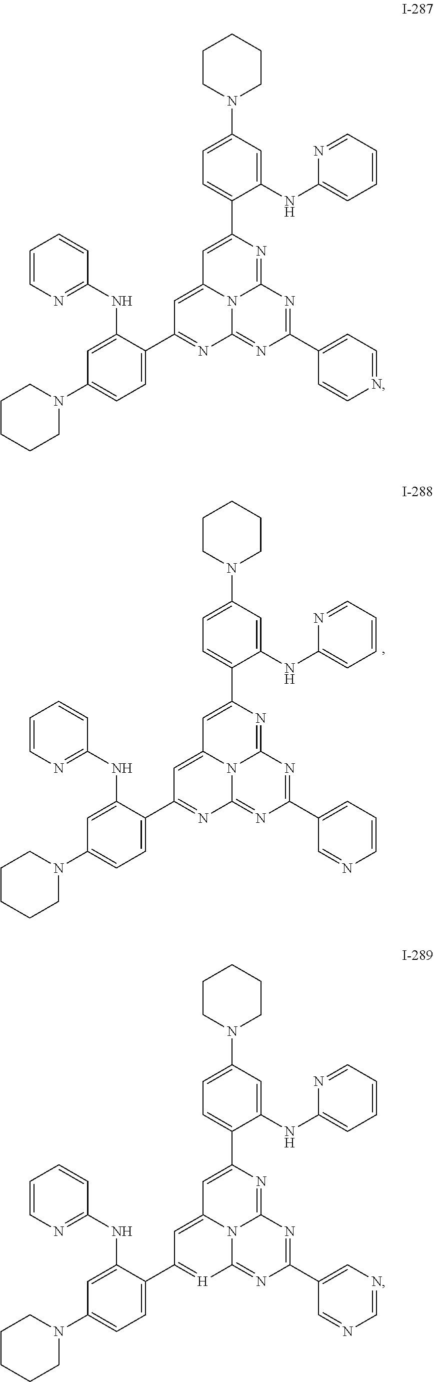

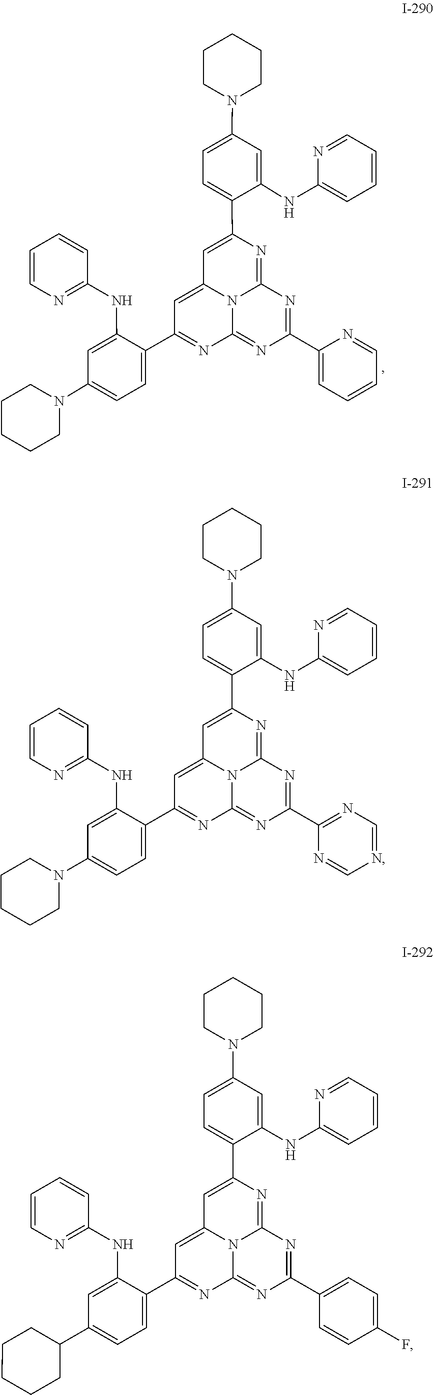 [SMILES: C1=C(c2ccc(N3CCCCC3)cc2Nc2ccccn2)N=C2N=C(c3ccccn3)N=C3N=C(c4ccc(N5CCCCC5)cc4Nc4ccccn4)C=C1N23 ; C1=C(c2ccc(N3CCCCC3)cc2Nc2ccccn2)N=C2N=C(c3ncncn3)N=C3N=C(c4ccc(N5CCCCC5)cc4Nc4ccccn4)C=C1N23 ; Fc1ccc(C2=NC3=NC(c4ccc(N5CCCCC5)cc4Nc4ccccn4)=CC4=CC(c5ccc(C6CCCCC6)cc5Nc5ccccn5)=NC(=N2)N43)cc1]

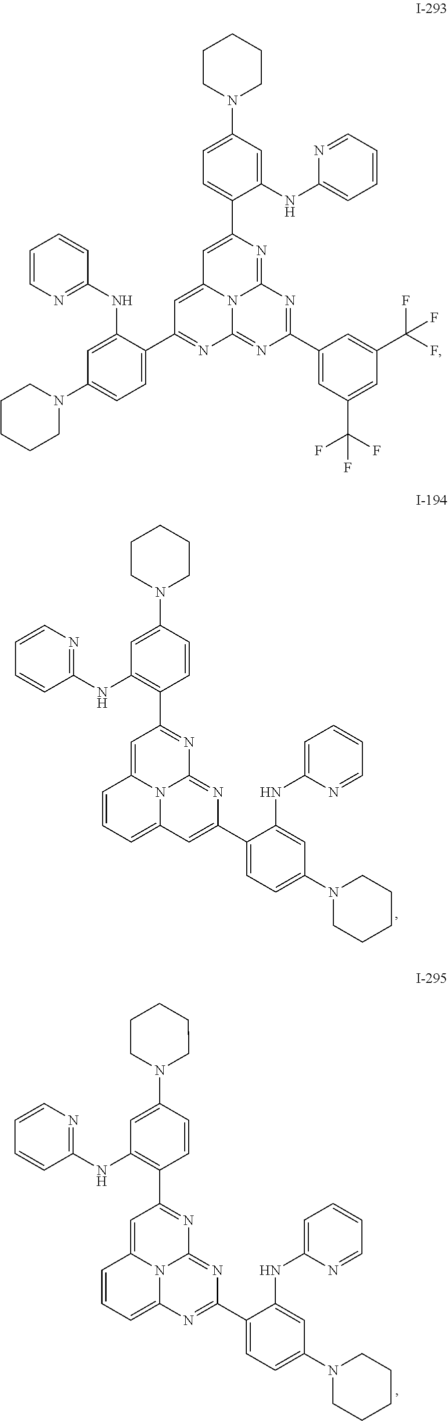 [SMILES: FC(F)(F)c1cc(C2=NC3=NC(c4ccc(N5CCCCC5)cc4Nc4ccccn4)=CC4=CC(c5ccc(N6CCCCC6)cc5Nc5ccccn5)=NC(=N2)N43)cc(C(F)(F)F)c1 ; C1=CC2=CC(c3ccc(N4CCCCC4)cc3Nc3ccccn3)=NC3=NC(c4ccc(N5CCCCC5)cc4Nc4ccccn4)=CC(=C1)N23 ; C1=CC2=CC(c3ccc(N4CCCCC4)cc3Nc3ccccn3)=NC3=NC(c4ccc(N5CCCCC5)cc4Nc4ccccn4)=NC(=C1)N23]

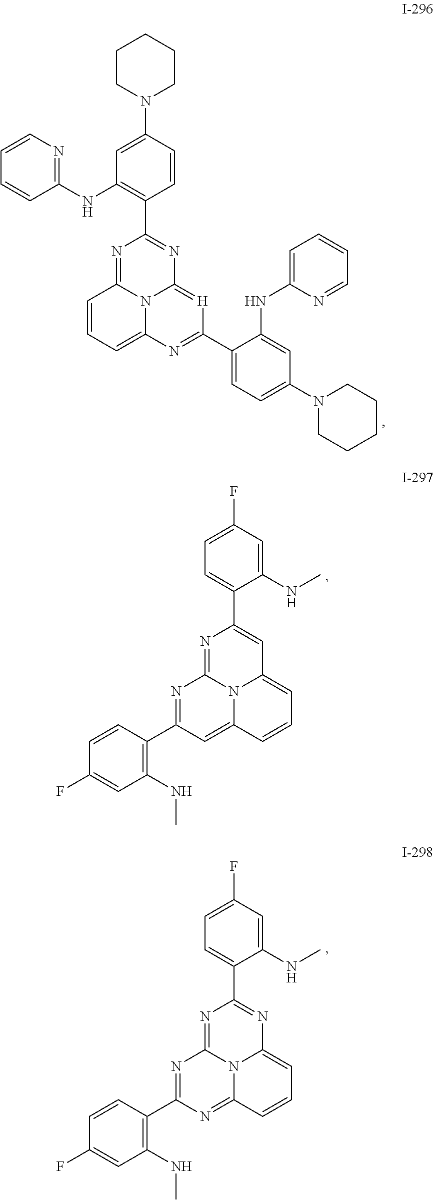

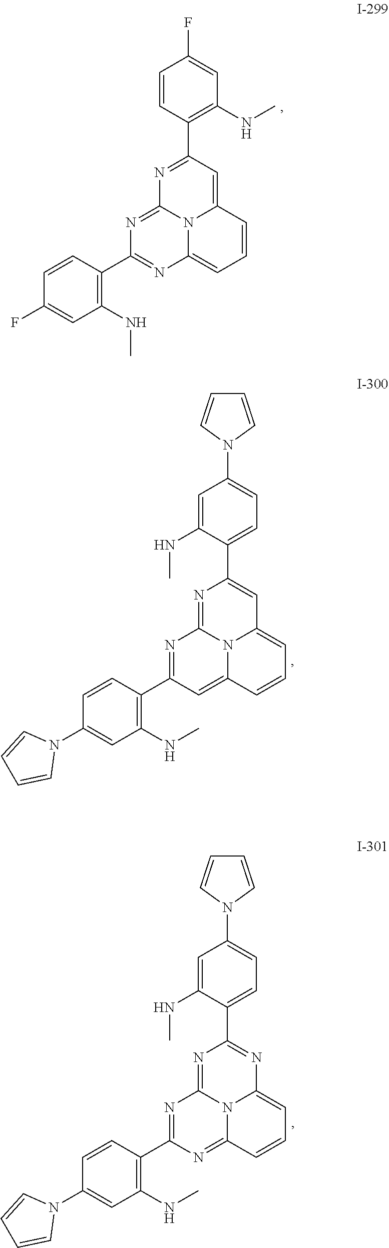 [SMILES: CNc1cc(F)ccc1C1=NC2=NC(c3ccc(F)cc3NC)=NC3=CC=CC(=C1)N32 ; CNc1cc(-n2cccc2)ccc1C1=CC2=CC=CC3=CC(c4ccc(-n5cccc5)cc4NC)=NC(=N1)N23 ; CNc1cc(-n2cccc2)ccc1C1=NC2=CC=CC3=NC(c4ccc(-n5cccc5)cc4NC)=NC(=N1)N23]

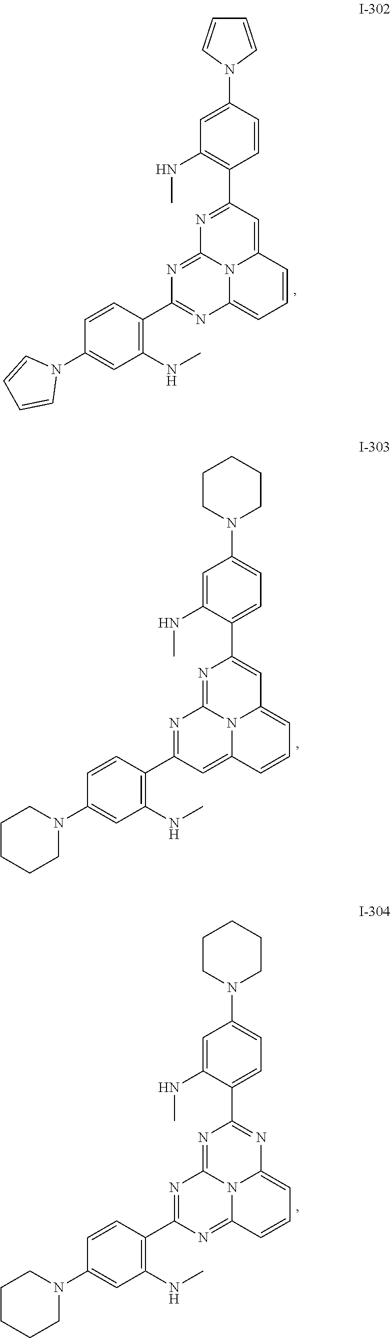 [SMILES: CNc1cc(-n2cccc2)ccc1C1=NC2=NC(c3ccc(-n4cccc4)cc3NC)=NC3=CC=CC(=C1)N32 ; CNc1cc(N2CCCCC2)ccc1C1=CC2=CC=CC3=CC(c4ccc(N5CCCCC5)cc4NC)=NC(=N1)N23 ; CNc1cc(N2CCCCC2)ccc1C1=NC2=CC=CC3=NC(c4ccc(N5CCCCC5)cc4NC)=NC(=N1)N23]

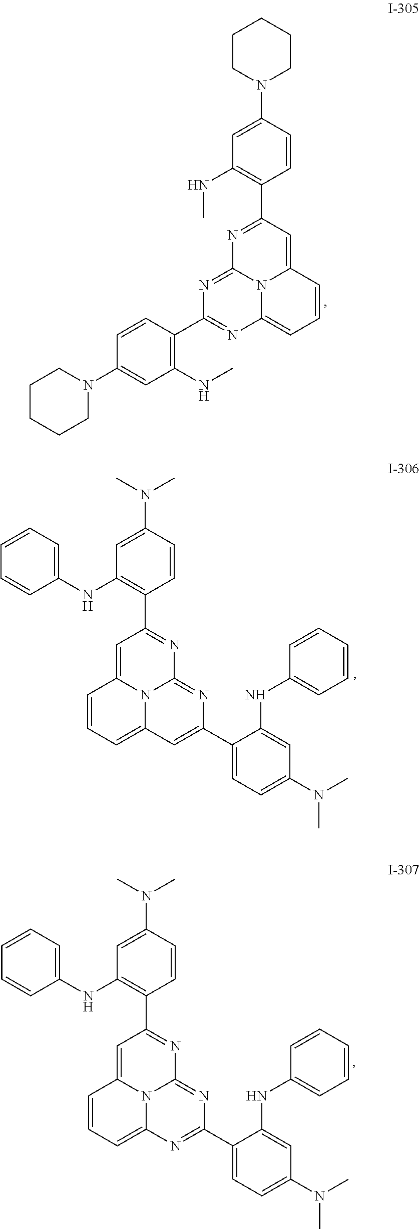 [SMILES: CNc1cc(N2CCCCC2)ccc1C1=NC2=NC(c3ccc(N4CCCCC4)cc3NC)=NC3=CC=CC(=C1)N32 ; CN(C)c1ccc(C2=CC3=CC=CC4=CC(c5ccc(N(C)C)cc5Nc5ccccc5)=NC(=N2)N34)c(Nc2ccccc2)c1 ; CN(C)c1ccc(C2=NC3=NC(c4ccc(N(C)C)cc4Nc4ccccc4)=NC4=CC=CC(=C2)N43)c(Nc2ccccc2)c1]

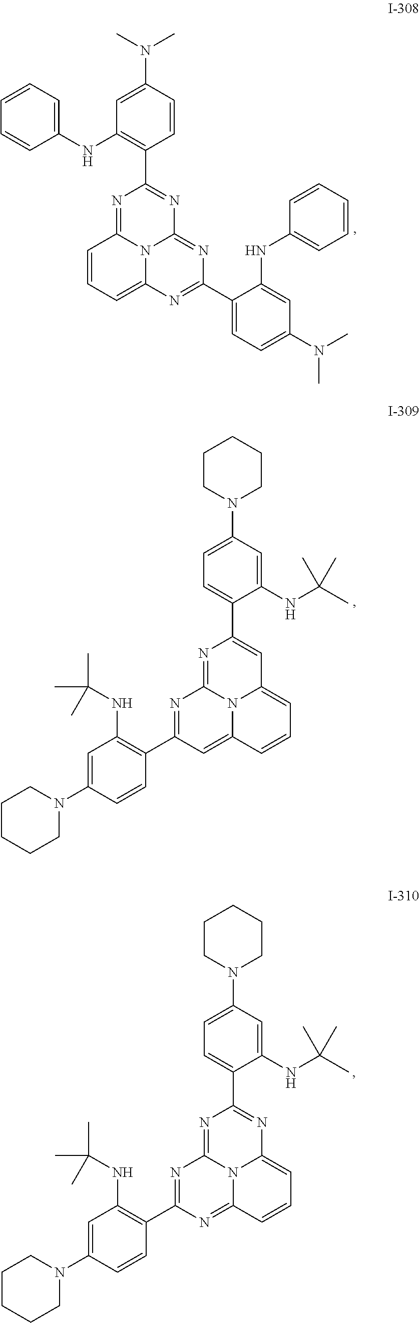 [SMILES: CN(C)c1ccc(C2=NC3=CC=CC4=NC(c5ccc(N(C)C)cc5Nc5ccccc5)=NC(=N2)N34)c(Nc2ccccc2)c1 ; CC(C)(C)Nc1cc(N2CCCCC2)ccc1C1=CC2=CC=CC3=CC(c4ccc(N5CCCCC5)cc4NC(C)(C)C)=NC(=N1)N23 ; CC(C)(C)Nc1cc(N2CCCCC2)ccc1C1=NC2=CC=CC3=NC(c4ccc(N5CCCCC5)cc4NC(C)(C)C)=NC(=N1)N23]

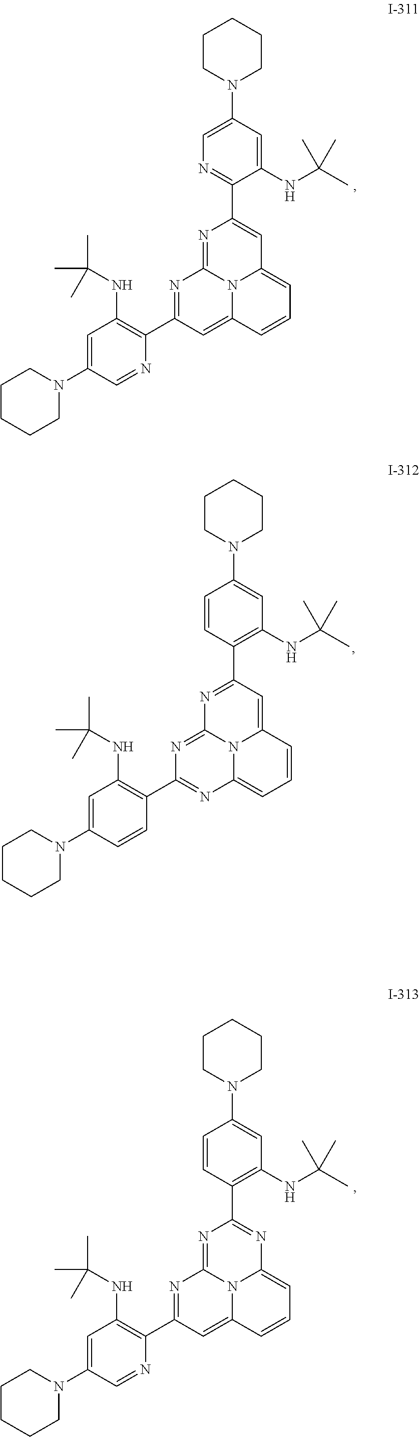 [SMILES: CC(C)(C)Nc1cc(N2CCCCC2)cnc1C1=CC2=CC=CC3=CC(c4ncc(N5CCCCC5)cc4NC(C)(C)C)=NC(=N1)N23 ; CC(C)(C)Nc1cc(N2CCCCC2)ccc1C1=NC2=NC(c3ccc(N4CCCCC4)cc3NC(C)(C)C)=NC3=CC=CC(=C1)N32 ; CC(C)(C)Nc1cc(N2CCCCC2)ccc1C1=NC2=CC=CC3=CC(c4ncc(N5CCCCC5)cc4NC(C)(C)C)=NC(=N1)N32]

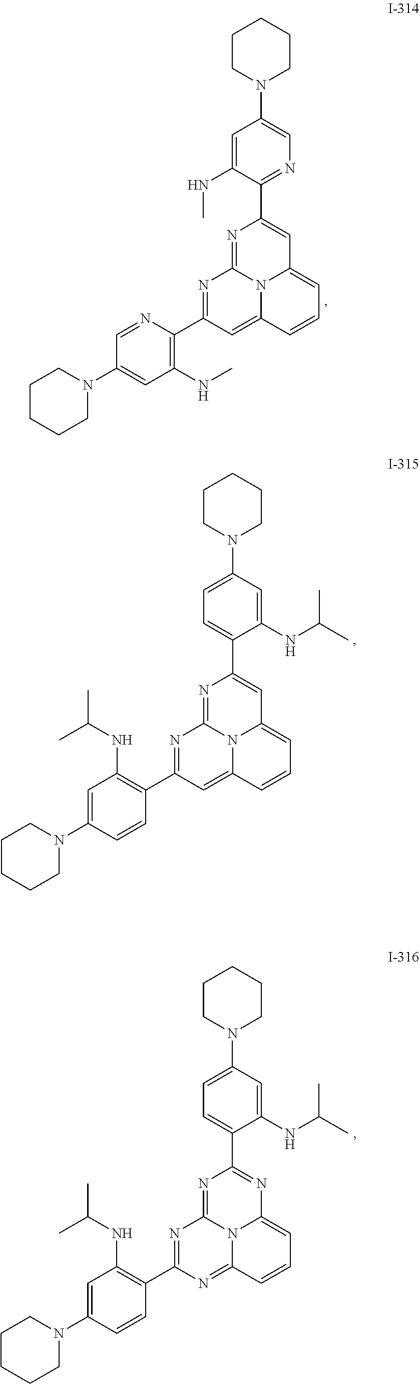 [SMILES: CNc1cc(N2CCCCC2)cnc1C1=CC2=CC=CC3=CC(c4ncc(N5CCCCC5)cc4NC)=NC(=N1)N23 ; CC(C)Nc1cc(N2CCCCC2)ccc1C1=CC2=CC=CC3=CC(c4ccc(N5CCCCC5)cc4NC(C)C)=NC(=N1)N23 ; CC(C)Nc1cc(N2CCCCC2)ccc1C1=NC2=CC=CC3=NC(c4ccc(N5CCCCC5)cc4NC(C)C)=NC(=N1)N23]

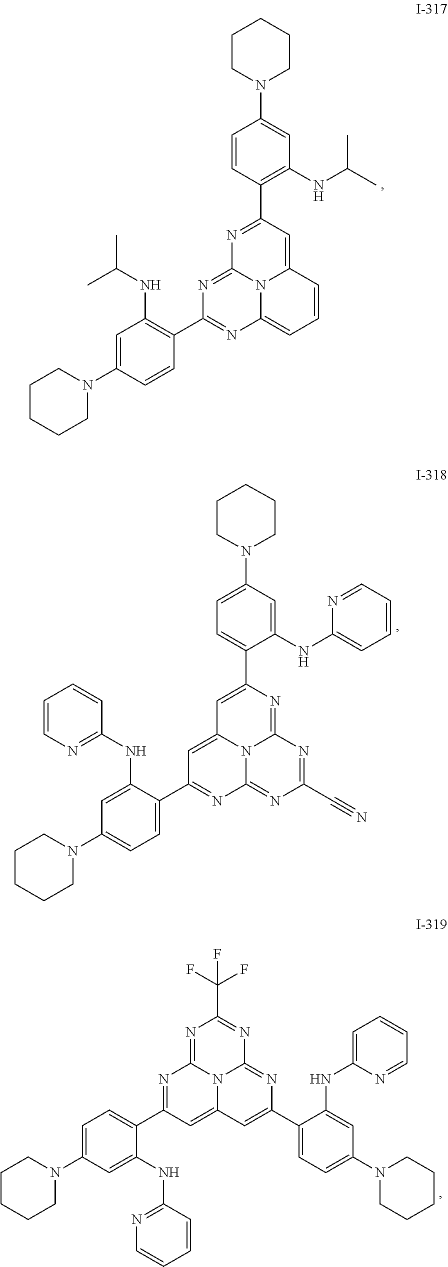 [SMILES: CC(C)Nc1cc(N2CCCCC2)ccc1C1=NC2=NC(c3ccc(N4CCCCC4)cc3NC(C)C)=NC3=CC=CC(=C1)N32 ; N#CC1=NC2=NC(c3ccc(N4CCCCC4)cc3Nc3ccccn3)=CC3=CC(c4ccc(N5CCCCC5)cc4Nc4ccccn4)=NC(=N1)N32 ; FC(F)(F)C1=NC2=NC(c3ccc(N4CCCCC4)cc3Nc3ccccn3)=CC3=CC(c4ccc(N5CCCCC5)cc4Nc4ccccn4)=NC(=N1)N32]

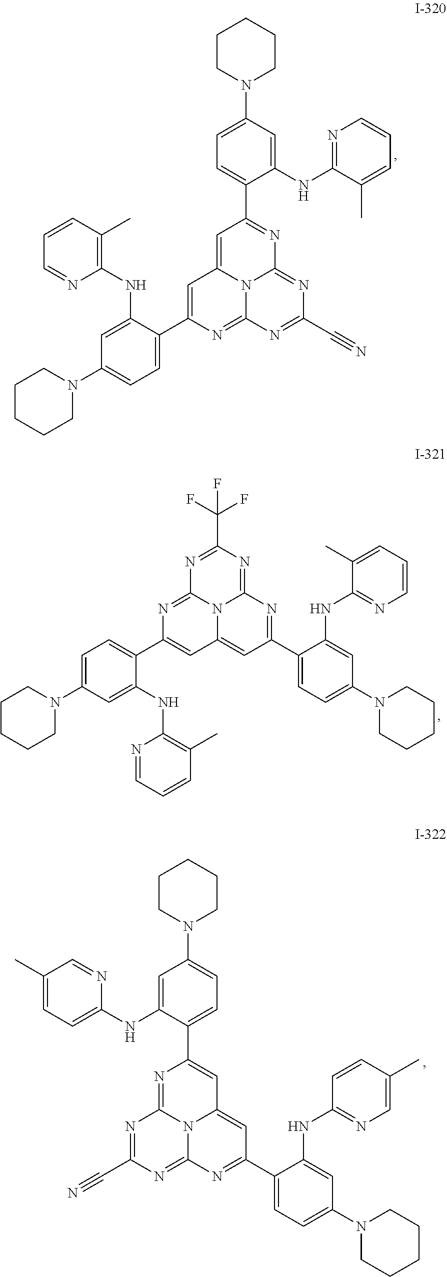 [SMILES: Cc1cccnc1Nc1cc(N2CCCCC2)ccc1C1=CC2=CC(c3ccc(N4CCCCC4)cc3Nc3ncccc3C)=NC3=NC(C#N)=NC(=N1)N23 ; Cc1cccnc1Nc1cc(N2CCCCC2)ccc1C1=CC2=CC(c3ccc(N4CCCCC4)cc3Nc3ncccc3C)=NC3=NC(C(F)(F)F)=NC(=N1)N23 ; Cc1ccc(Nc2cc(N3CCCCC3)ccc2C2=CC3=CC(c4ccc(N5CCCCC5)cc4Nc4ccc(C)cn4)=NC4=NC(C#N)=NC(=N2)N34)nc1]

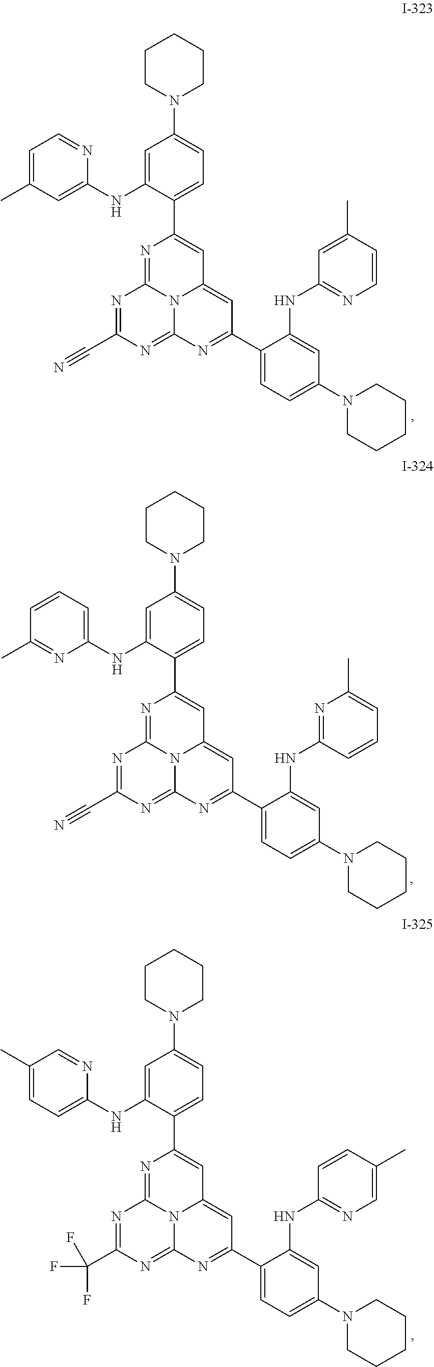

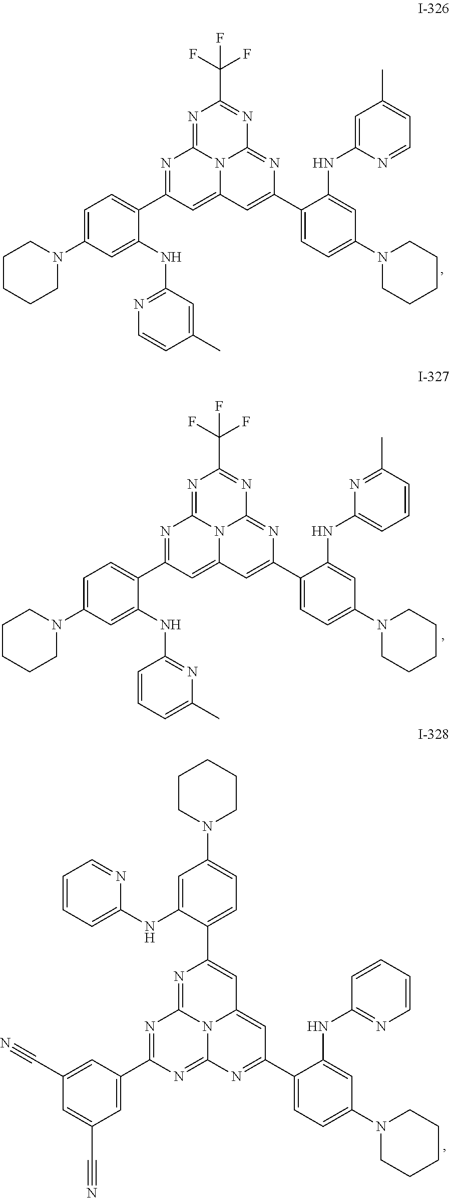

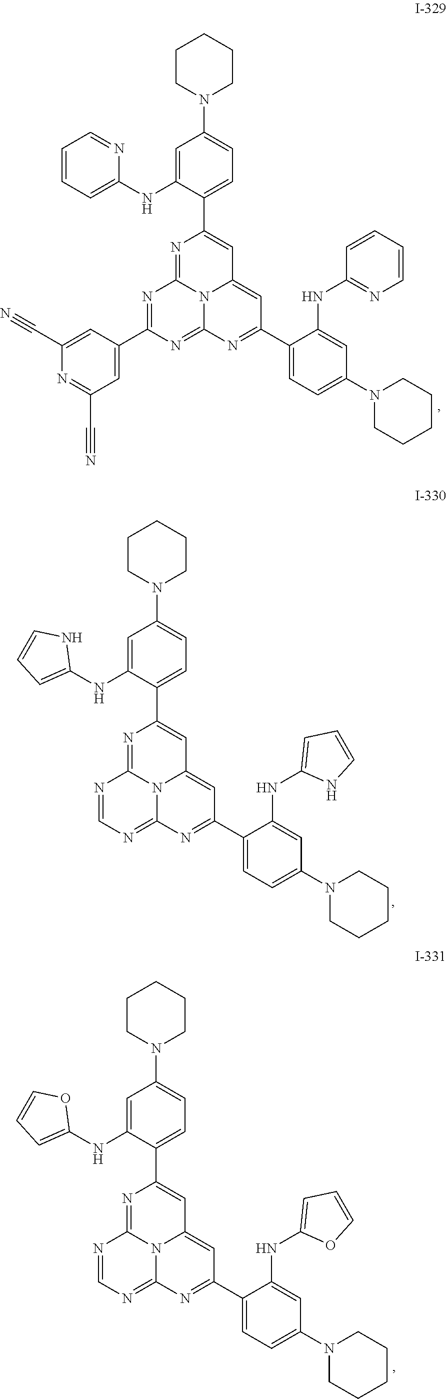 [SMILES: N#Cc1cc(C2=NC3=NC(c4ccc(N5CCCCC5)cc4Nc4ccccn4)=CC4=CC(c5ccc(N6CCCCC6)cc5Nc5ccccn5)=NC(=N2)N43)cc(C#N)n1 ; C1=NC2=NC(c3ccc(N4CCCCC4)cc3Nc3ccc[nH]3)=CC3=CC(c4ccc(N5CCCCC5)cc4Nc4ccc[nH]4)=NC(=N1)N32 ; C1=NC2=NC(c3ccc(N4CCCCC4)cc3Nc3ccco3)=CC3=CC(c4ccc(N5CCCCC5)cc4Nc4ccco4)=NC(=N1)N32]

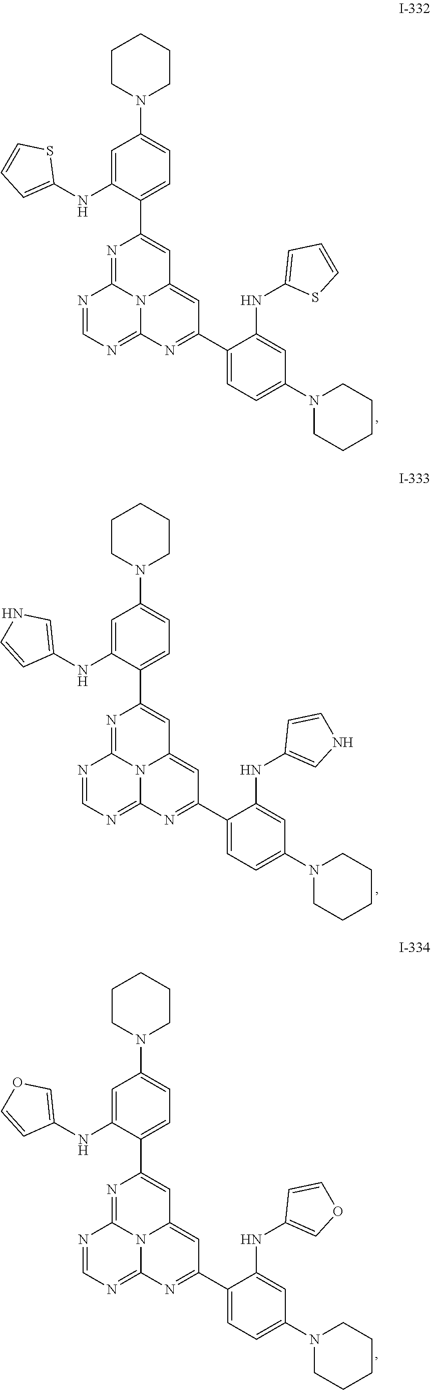 [SMILES: C1=NC2=NC(c3ccc(N4CCCCC4)cc3Nc3cccs3)=CC3=CC(c4ccc(N5CCCCC5)cc4Nc4cccs4)=NC(=N1)N32 ; C1=NC2=NC(c3ccc(N4CCCCC4)cc3Nc3cc[nH]c3)=CC3=CC(c4ccc(N5CCCCC5)cc4Nc4cc[nH]c4)=NC(=N1)N32 ; C1=NC2=NC(c3ccc(N4CCCCC4)cc3Nc3ccoc3)=CC3=CC(c4ccc(N5CCCCC5)cc4Nc4ccoc4)=NC(=N1)N32]

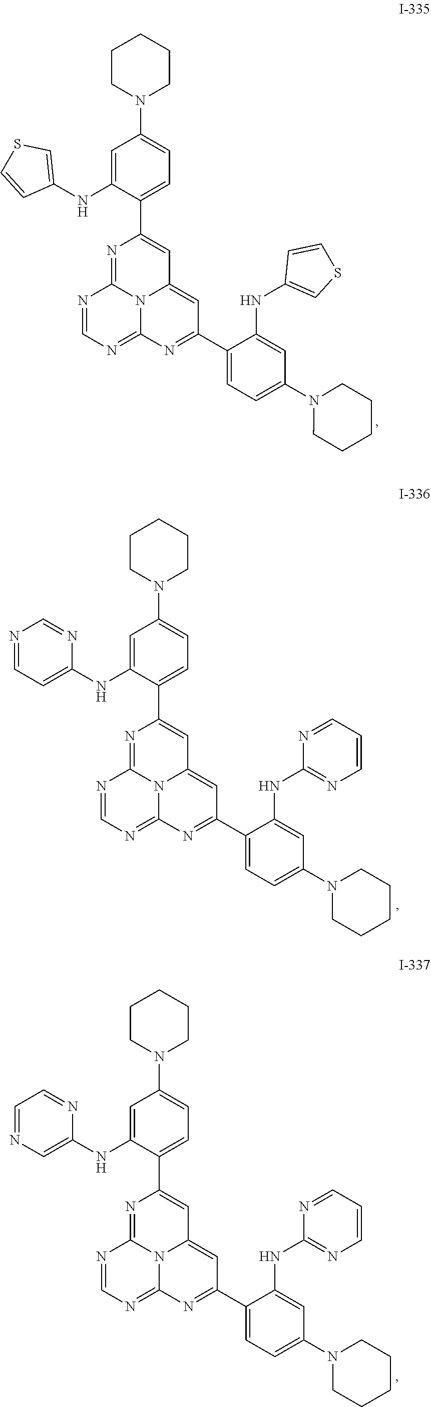 [SMILES: C1=NC2=NC(c3ccc(N4CCCCC4)cc3Nc3ccsc3)=CC3=CC(c4ccc(N5CCCCC5)cc4Nc4ccsc4)=NC(=N1)N32 ; C1=NC2=NC(c3ccc(N4CCCCC4)cc3Nc3ccncn3)=CC3=CC(c4ccc(N5CCCCC5)cc4Nc4ncccn4)=NC(=N1)N32 ; C1=NC2=NC(c3ccc(N4CCCCC4)cc3Nc3cnccn3)=CC3=CC(c4ccc(N5CCCCC5)cc4Nc4ncccn4)=NC(=N1)N32]

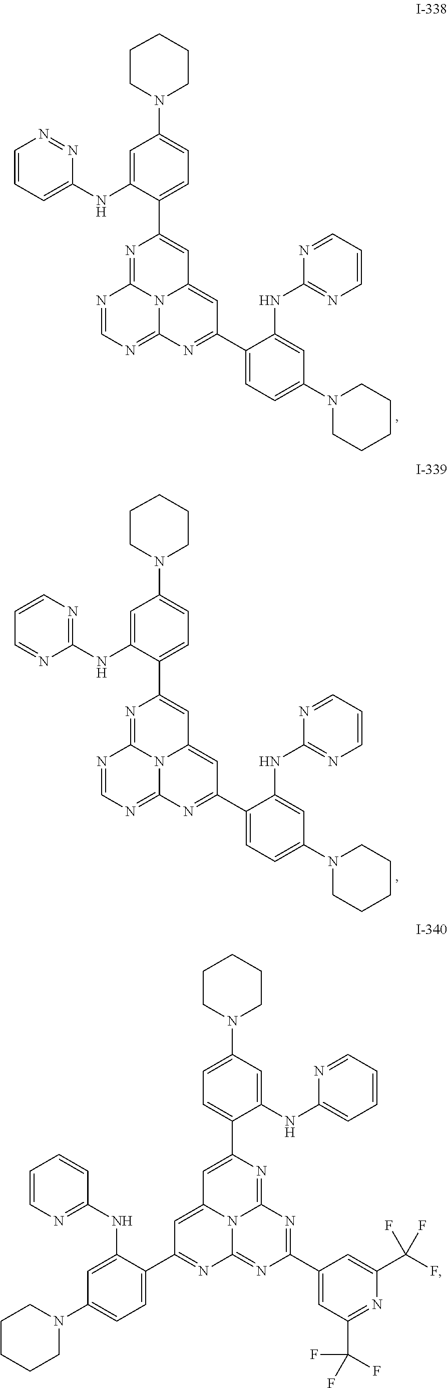 [SMILES: C1=NC2=NC(c3ccc(N4CCCCC4)cc3Nc3cccnn3)=CC3=CC(c4ccc(N5CCCCC5)cc4Nc4ncccn4)=NC(=N1)N32 ; C1=NC2=NC(c3ccc(N4CCCCC4)cc3Nc3ncccn3)=CC3=CC(c4ccc(N5CCCCC5)cc4Nc4ncccn4)=NC(=N1)N32 ; FC(F)(F)c1cc(C2=NC3=NC(c4ccc(N5CCCCC5)cc4Nc4ccccn4)=CC4=CC(c5ccc(N6CCCCC6)cc5Nc5ccccn5)=NC(=N2)N43)cc(C(F)(F)F)n1]

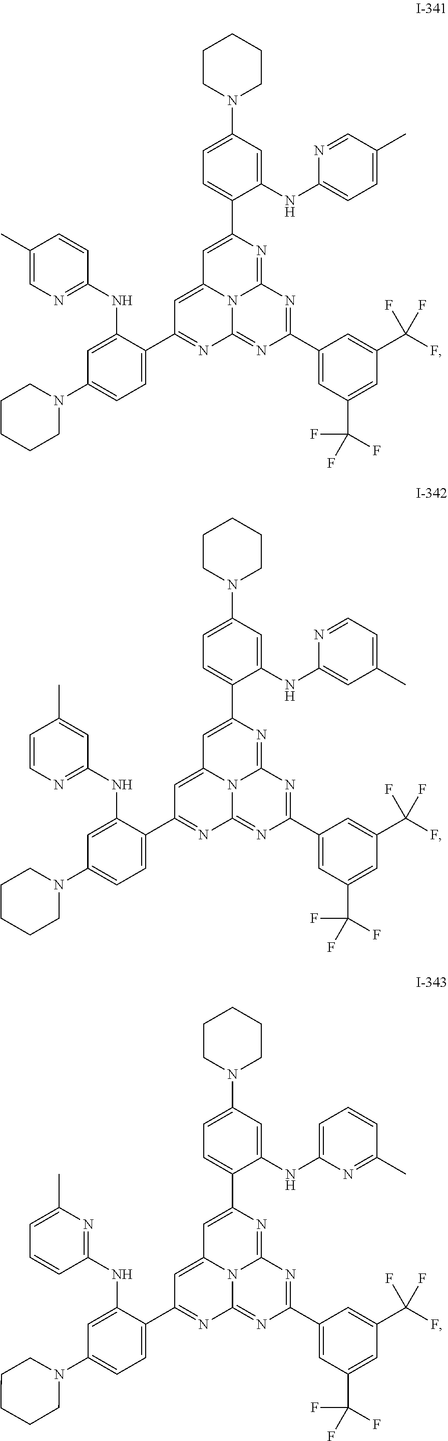 [SMILES: Cc1ccc(Nc2cc(N3CCCCC3)ccc2C2=CC3=CC(c4ccc(N5CCCCC5)cc4Nc4ccc(C)cn4)=NC4=NC(c5cc(C(F)(F)F)cc(C(F)(F)F)c5)=NC(=N2)N34)nc1 ; Cc1ccnc(Nc2cc(N3CCCCC3)ccc2C2=CC3=CC(c4ccc(N5CCCCC5)cc4Nc4cc(C)ccn4)=NC4=NC(c5cc(C(F)(F)F)cc(C(F)(F)F)c5)=NC(=N2)N34)c1 ; Cc1cccc(Nc2cc(N3CCCCC3)ccc2C2=CC3=CC(c4ccc(N5CCCCC5)cc4Nc4cccc(C)n4)=NC4=NC(c5cc(C(F)(F)F)cc(C(F)(F)F)c5)=NC(=N2)N34)n1]

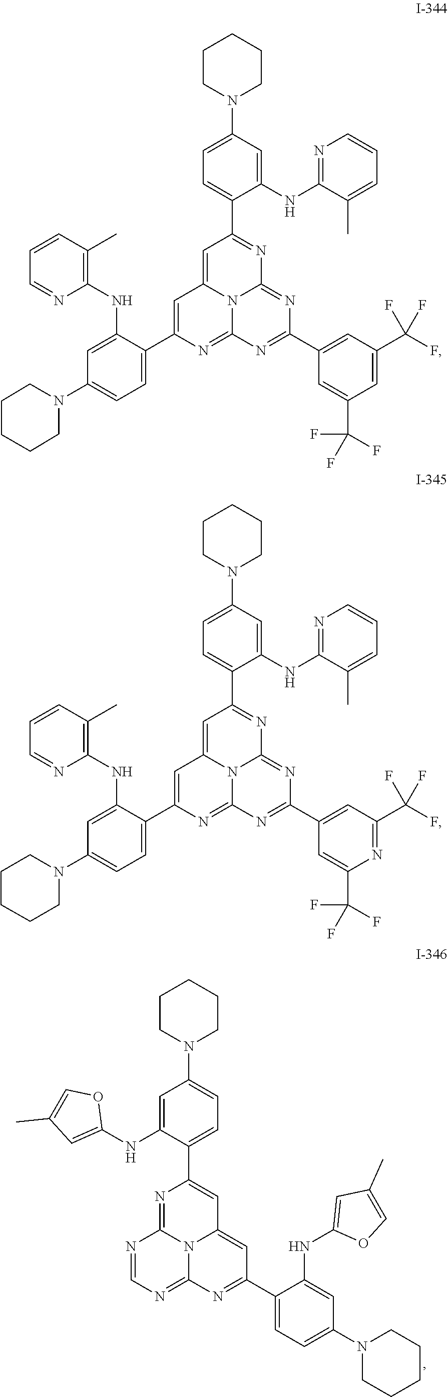 [SMILES: Cc1cccnc1Nc1cc(N2CCCCC2)ccc1C1=CC2=CC(c3ccc(N4CCCCC4)cc3Nc3ncccc3C)=NC3=NC(c4cc(C(F)(F)F)cc(C(F)(F)F)c4)=NC(=N1)N23 ; Cc1cccnc1Nc1cc(N2CCCCC2)ccc1C1=CC2=CC(c3ccc(N4CCCCC4)cc3Nc3ncccc3C)=NC3=NC(c4cc(C(F)(F)F)nc(C(F)(F)F)c4)=NC(=N1)N23 ; Cc1coc(Nc2cc(N3CCCCC3)ccc2C2=CC3=CC(c4ccc(N5CCCCC5)cc4Nc4cc(C)co4)=NC4=NC=NC(=N2)N34)c1]

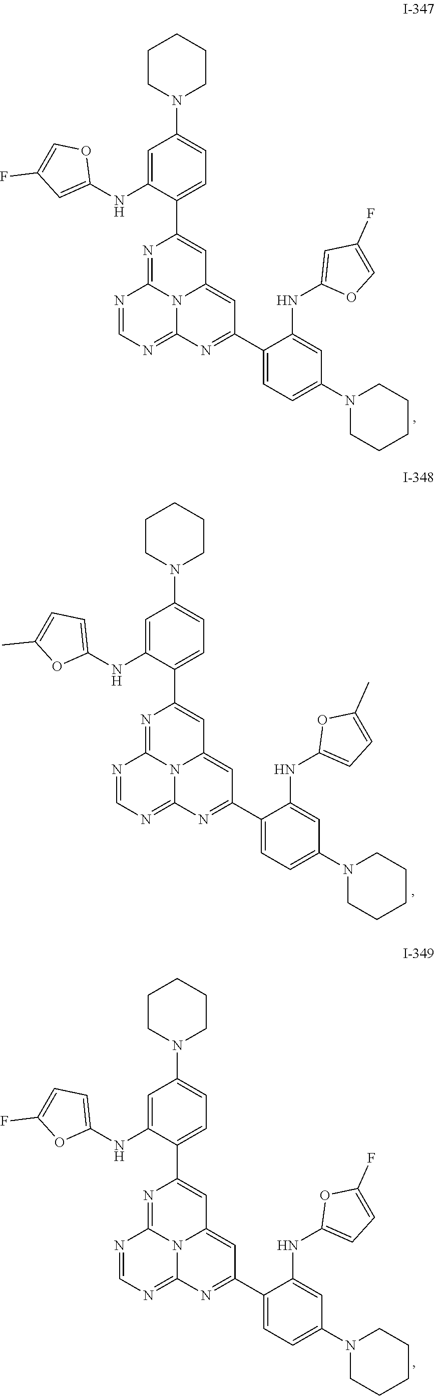 [SMILES: Fc1coc(Nc2cc(N3CCCCC3)ccc2C2=CC3=CC(c4ccc(N5CCCCC5)cc4Nc4cc(F)co4)=NC4=NC=NC(=N2)N34)c1 ; Cc1ccc(Nc2cc(N3CCCCC3)ccc2C2=CC3=CC(c4ccc(N5CCCCC5)cc4Nc4ccc(C)o4)=NC4=NC=NC(=N2)N34)o1 ; Fc1ccc(Nc2cc(N3CCCCC3)ccc2C2=CC3=CC(c4ccc(N5CCCCC5)cc4Nc4ccc(F)o4)=NC4=NC=NC(=N2)N34)o1]

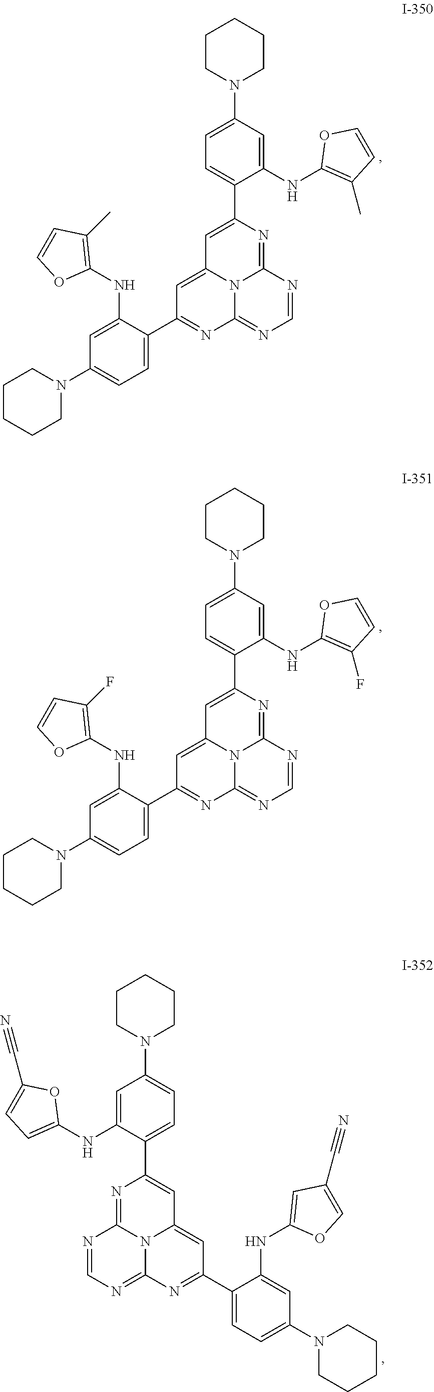 [SMILES: Cc1ccoc1Nc1cc(N2CCCCC2)ccc1C1=CC2=CC(c3ccc(N4CCCCC4)cc3Nc3occc3C)=NC3=NC=NC(=N1)N23 ; Fc1ccoc1Nc1cc(N2CCCCC2)ccc1C1=CC2=CC(c3ccc(N4CCCCC4)cc3Nc3occc3F)=NC3=NC=NC(=N1)N23 ; N#Cc1coc(Nc2cc(N3CCCCC3)ccc2C2=NC3=NC=NC4=NC(c5ccc(N6CCCCC6)cc5Nc5ccc(C#N)o5)=CC(=C2)N34)c1]

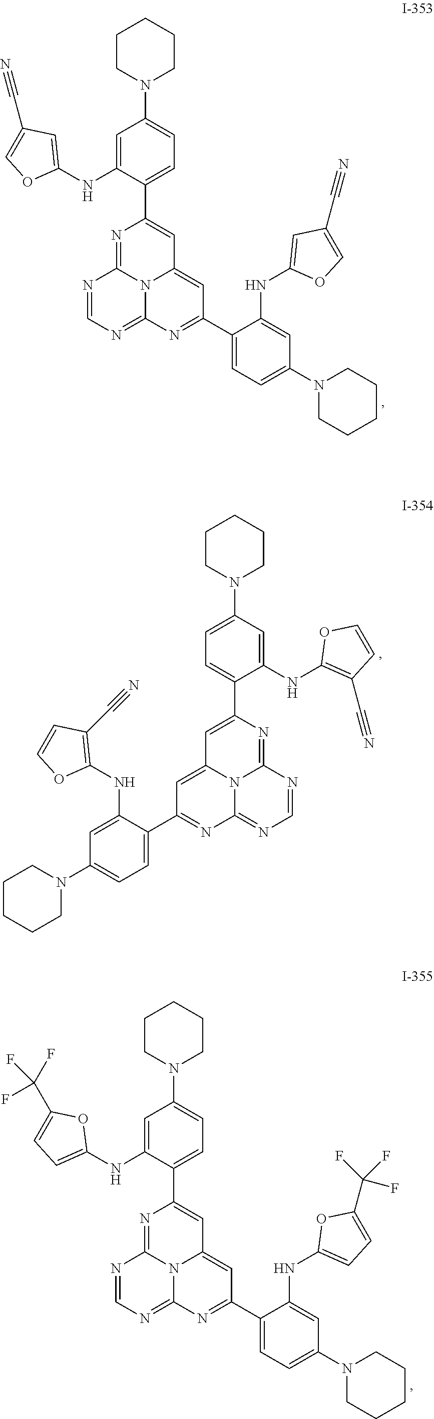 [SMILES: N#Cc1coc(Nc2cc(N3CCCCC3)ccc2C2=CC3=CC(c4ccc(N5CCCCC5)cc4Nc4cc(C#N)co4)=NC4=NC=NC(=N2)N34)c1 ; N#Cc1ccoc1Nc1cc(N2CCCCC2)ccc1C1=CC2=CC(c3ccc(N4CCCCC4)cc3Nc3occc3C#N)=NC3=NC=NC(=N1)N23 ; FC(F)(F)c1ccc(Nc2cc(N3CCCCC3)ccc2C2=CC3=CC(c4ccc(N5CCCCC5)cc4Nc4ccc(C(F)(F)F)o4)=NC4=NC=NC(=N2)N34)o1]

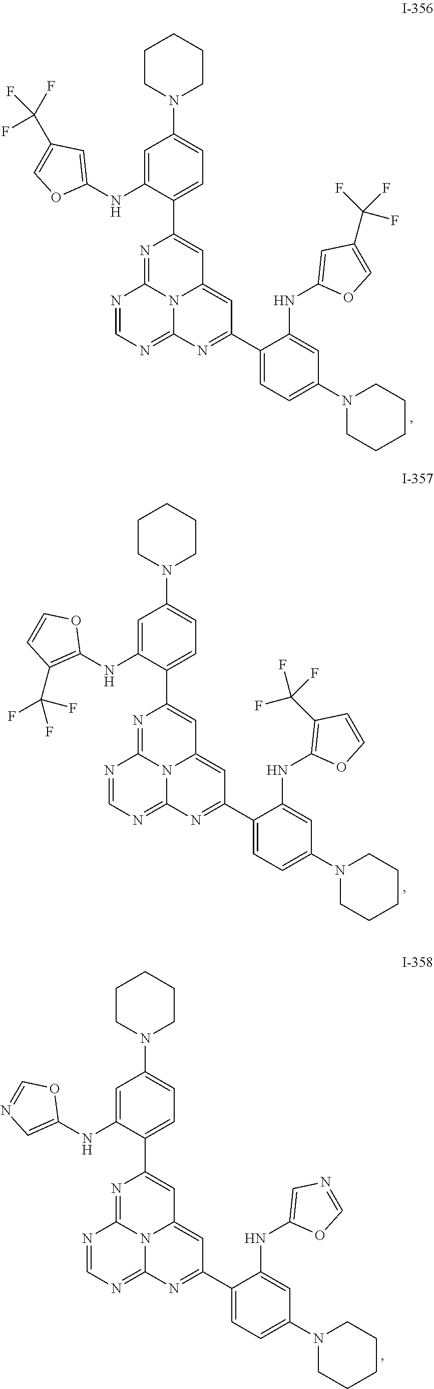 [SMILES: FC(F)(F)c1coc(Nc2cc(N3CCCCC3)ccc2C2=CC3=CC(c4ccc(N5CCCCC5)cc4Nc4cc(C(F)(F)F)co4)=NC4=NC=NC(=N2)N34)c1 ; FC(F)(F)c1ccoc1Nc1cc(N2CCCCC2)ccc1C1=CC2=CC(c3ccc(N4CCCCC4)cc3Nc3occc3C(F)(F)F)=NC3=NC=NC(=N1)N23 ; C1=NC2=NC(c3ccc(N4CCCCC4)cc3Nc3cnco3)=CC3=CC(c4ccc(N5CCCCC5)cc4Nc4cnco4)=NC(=N1)N32]

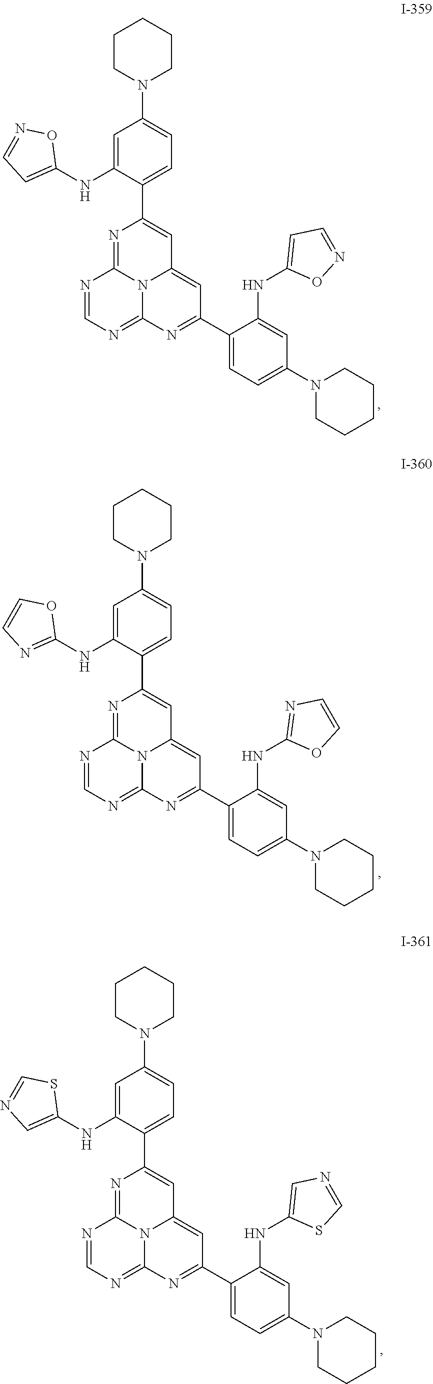

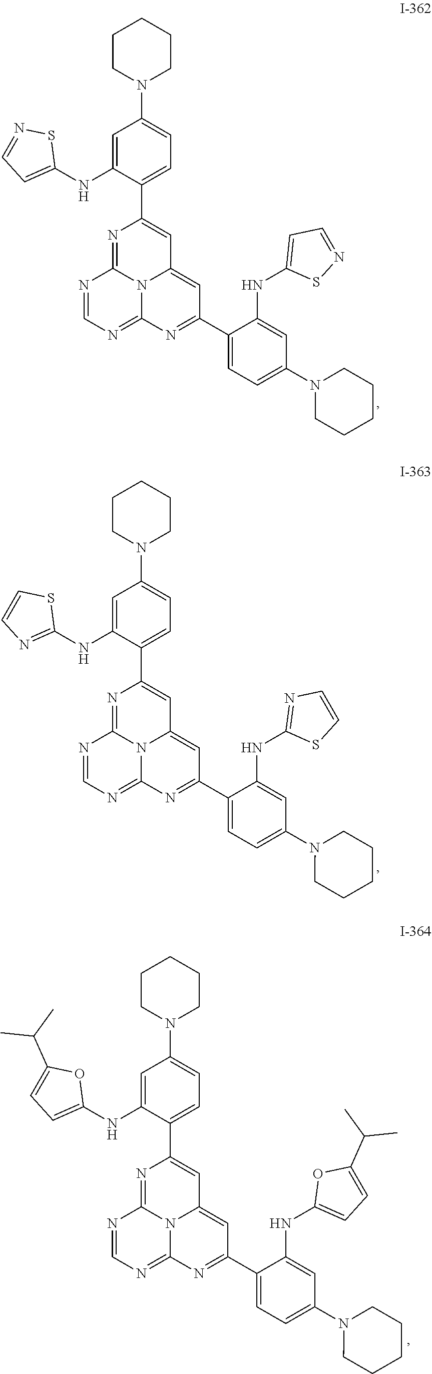 [SMILES: C1=NC2=NC(c3ccc(N4CCCCC4)cc3Nc3ccns3)=CC3=CC(c4ccc(N5CCCCC5)cc4Nc4ccns4)=NC(=N1)N32 ; C1=NC2=NC(c3ccc(N4CCCCC4)cc3Nc3nccs3)=CC3=CC(c4ccc(N5CCCCC5)cc4Nc4nccs4)=NC(=N1)N32 ; CC(C)c1ccc(Nc2cc(N3CCCCC3)ccc2C2=CC3=CC(c4ccc(N5CCCCC5)cc4Nc4ccc(C(C)C)o4)=NC4=NC=NC(=N2)N34)o1]

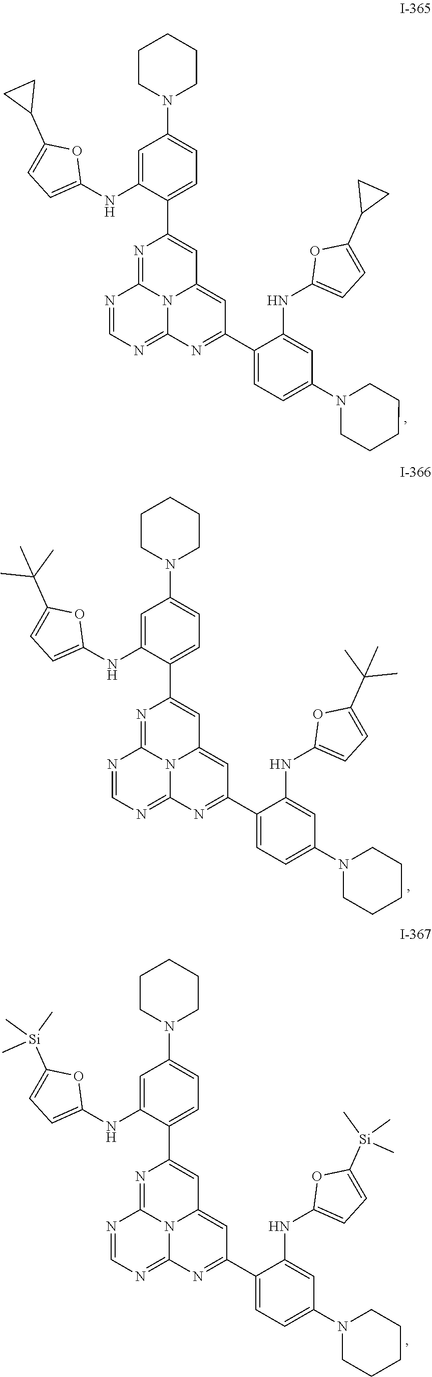 [SMILES: C1=NC2=NC(c3ccc(N4CCCCC4)cc3Nc3ccc(C4CC4)o3)=CC3=CC(c4ccc(N5CCCCC5)cc4Nc4ccc(C5CC5)o4)=NC(=N1)N32 ; CC(C)(C)c1ccc(Nc2cc(N3CCCCC3)ccc2C2=CC3=CC(c4ccc(N5CCCCC5)cc4Nc4ccc(C(C)(C)C)o4)=NC4=NC=NC(=N2)N34)o1 ; C[Si](C)(C)c1ccc(Nc2cc(N3CCCCC3)ccc2C2=CC3=CC(c4ccc(N5CCCCC5)cc4Nc4ccc([Si](C)(C)C)o4)=NC4=NC=NC(=N2)N34)o1]

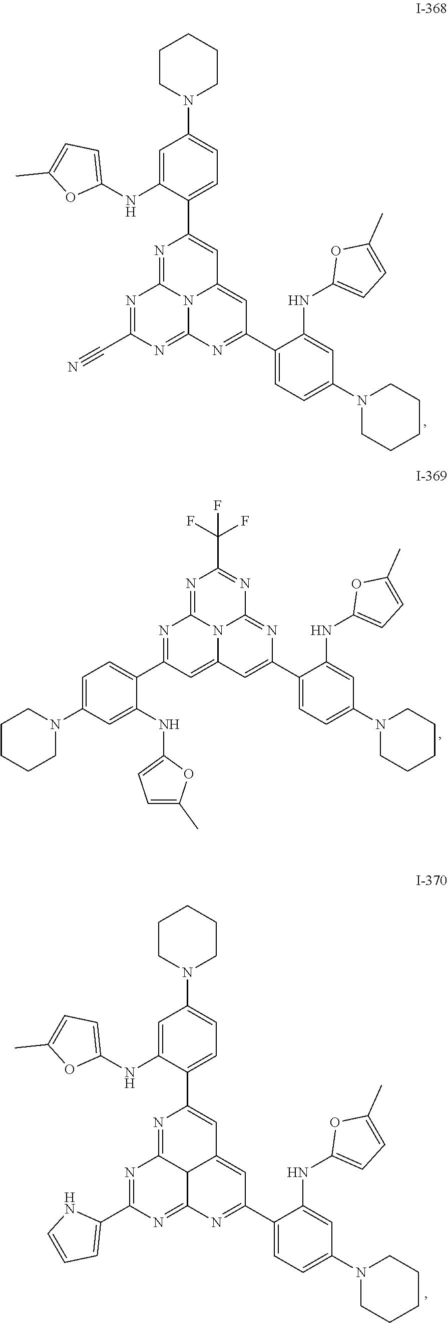 [SMILES: Cc1ccc(Nc2cc(N3CCCCC3)ccc2C2=CC3=CC(c4ccc(N5CCCCC5)cc4Nc4ccc(C)o4)=NC4=NC(C#N)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(C(F)(F)F)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ccc[nH]5)=NC(=N2)C34)o1]

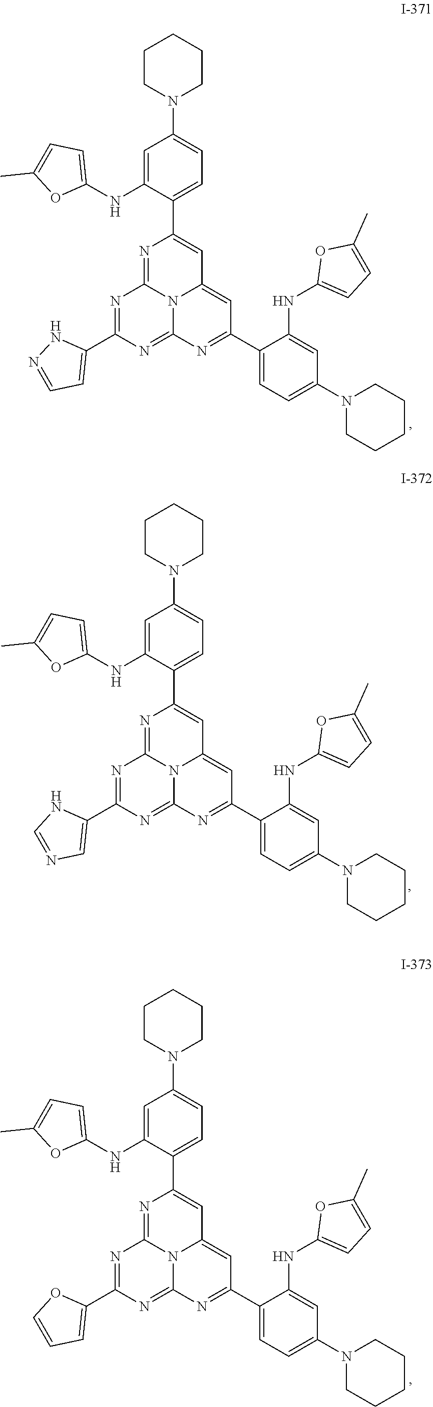

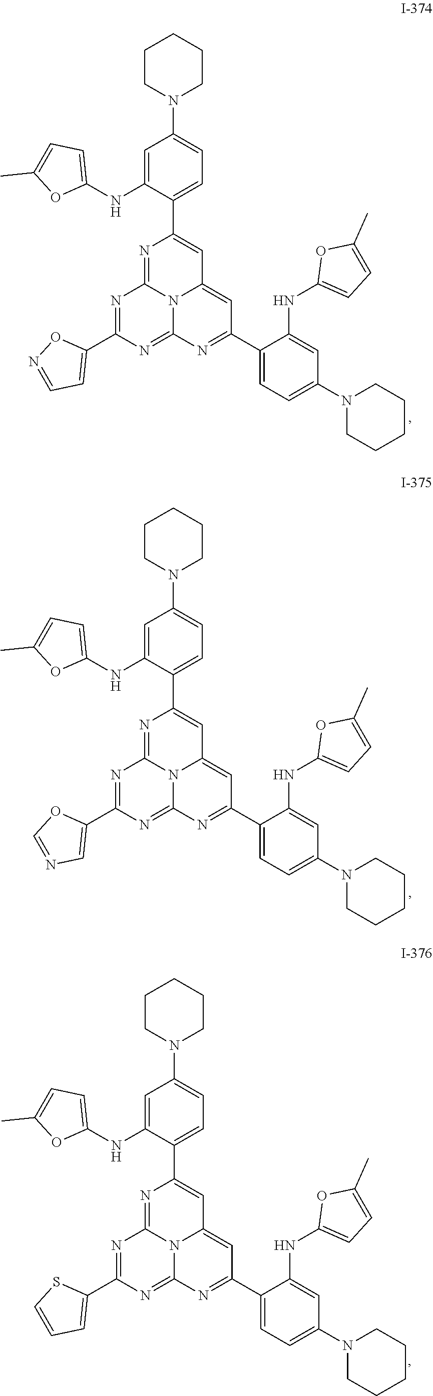

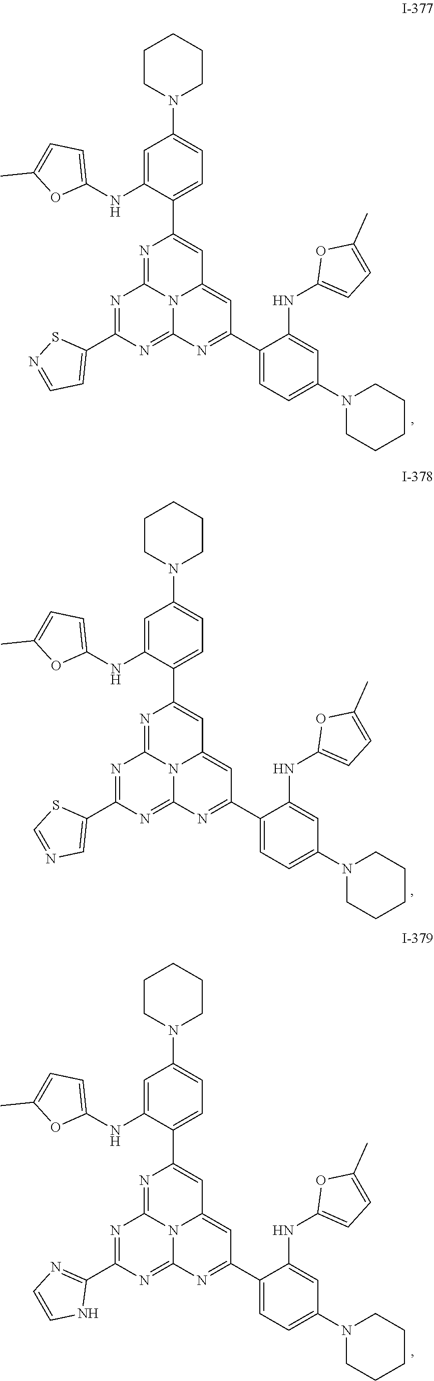 [SMILES: Cc1ccc(Nc2cc(N3CCCCC3)ccc2C2=CC3=CC(c4ccc(N5CCCCC5)cc4Nc4ccc(C)o4)=NC4=NC(c5ccns5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5cncs5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ncc[nH]5)=NC(=N2)N34)o1]

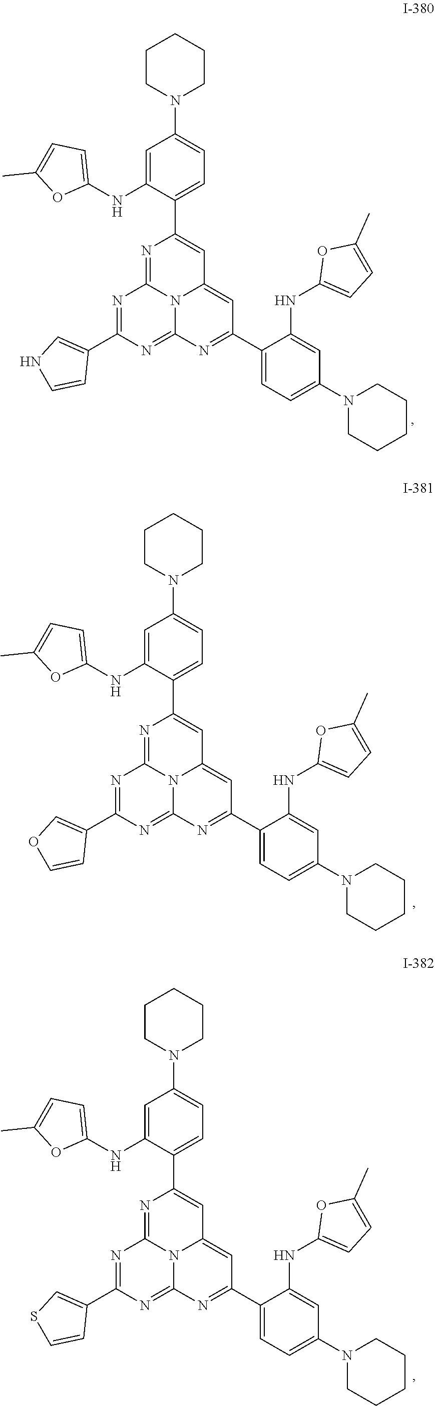 [SMILES: Cc1ccc(Nc2cc(N3CCCCC3)ccc2C2=CC3=CC(c4ccc(N5CCCCC5)cc4Nc4ccc(C)o4)=NC4=NC(c5cc[nH]c5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ccoc5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ccsc5)=NC(=N2)N34)o1]

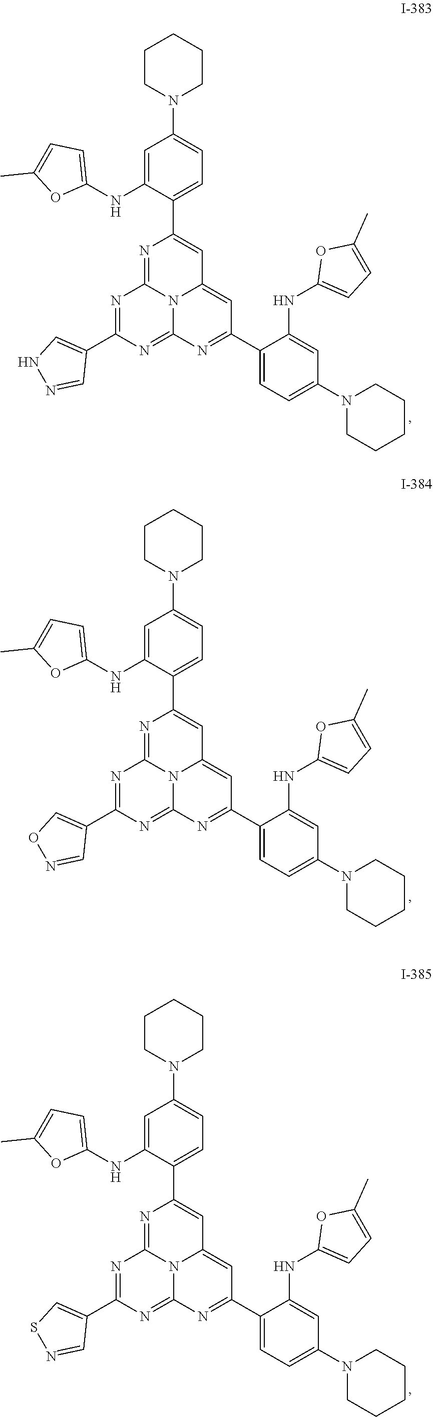 [SMILES: Cc1ccc(Nc2cc(N3CCCCC3)ccc2C2=CC3=CC(c4ccc(N5CCCCC5)cc4Nc4ccc(C)o4)=NC4=NC(c5cn[nH]c5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5cnoc5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5cnsc5)=NC(=N2)N34)o1]

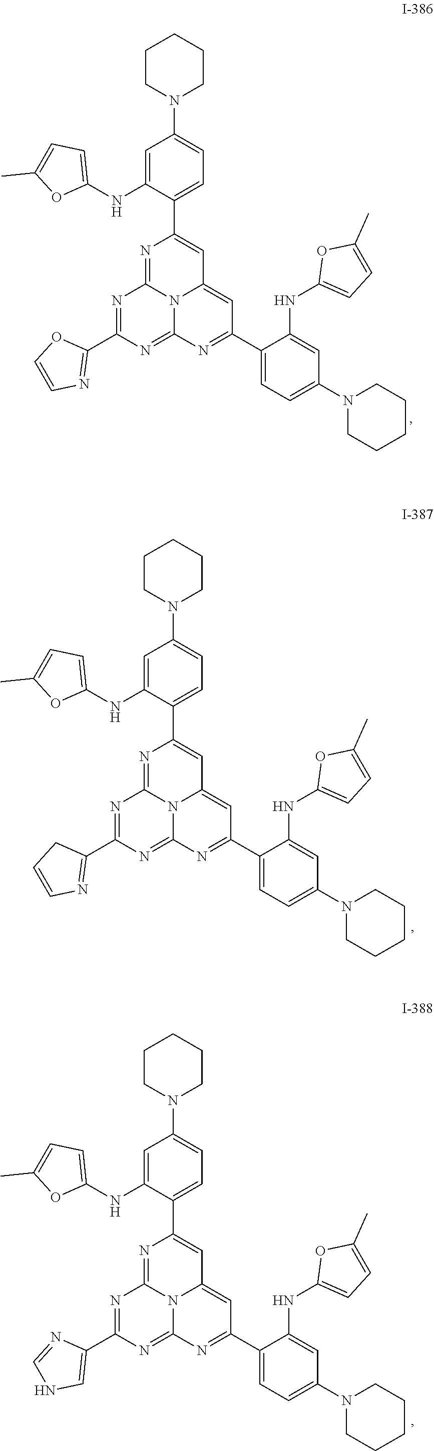 [SMILES: Cc1ccc(Nc2cc(N3CCCCC3)ccc2C2=CC3=CC(c4ccc(N5CCCCC5)cc4Nc4ccc(C)o4)=NC4=NC(c5ncco5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(C5=NC=CC5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5c[nH]cn5)=NC(=N2)N34)o1]

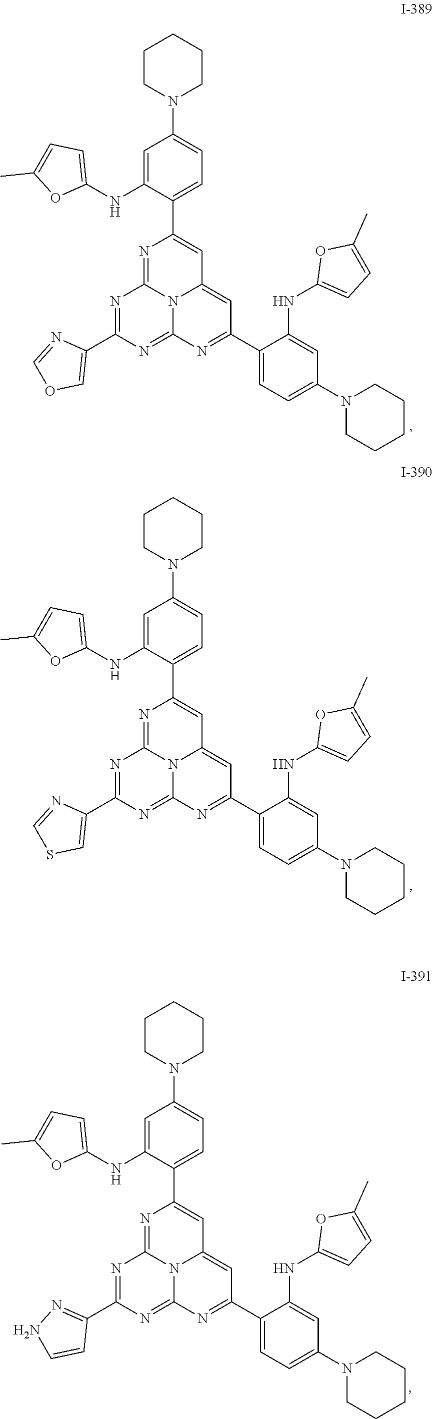

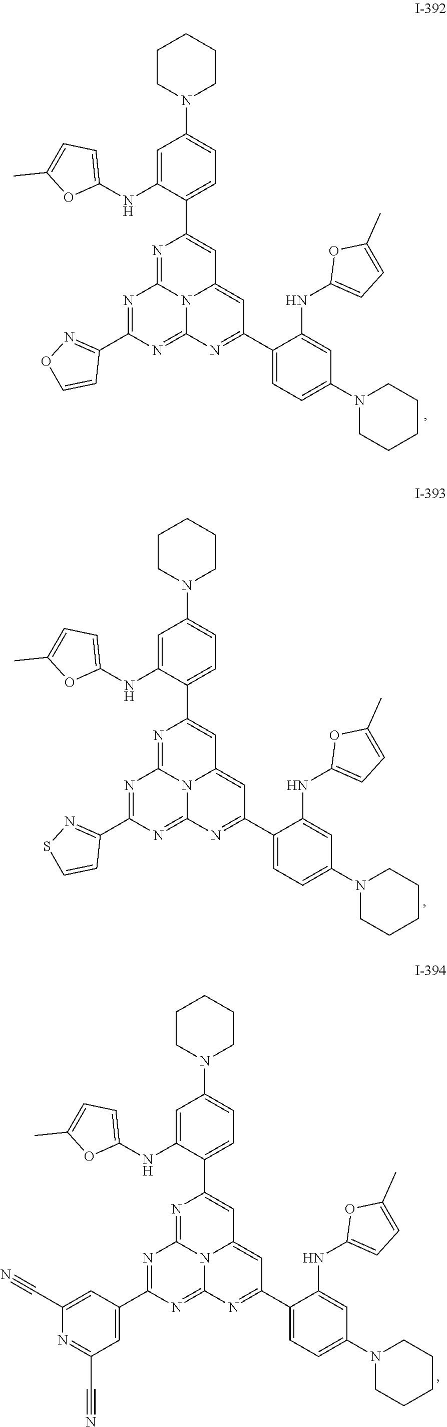 [SMILES: Cc1ccc(Nc2cc(N3CCCCC3)ccc2C2=CC3=CC(c4ccc(N5CCCCC5)cc4Nc4ccc(C)o4)=NC4=NC(c5ccon5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ccsn5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5cc(C#N)nc(C#N)c5)=NC(=N2)N34)o1]

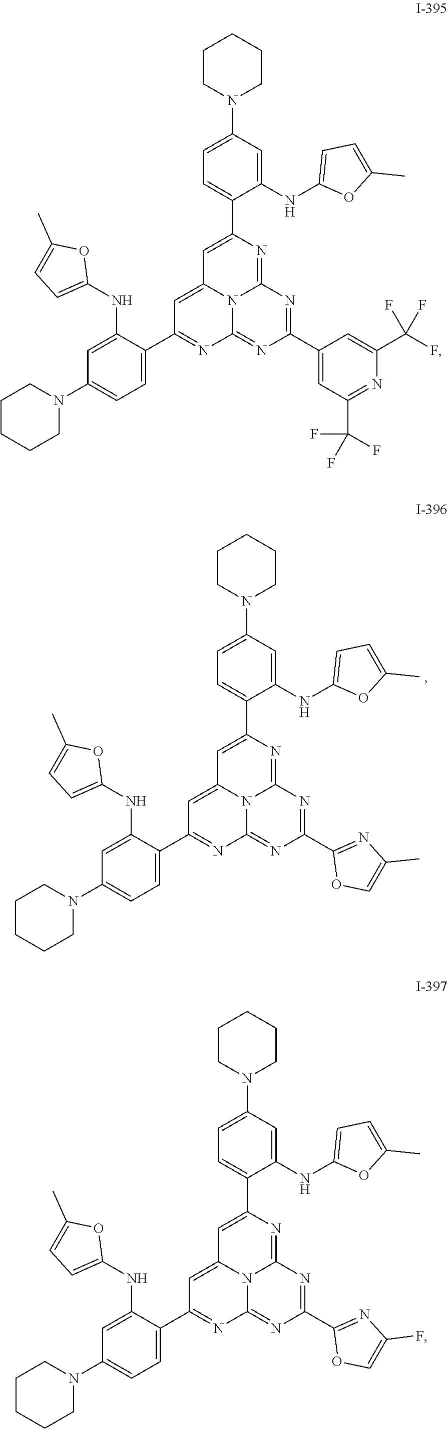 [SMILES: Cc1ccc(Nc2cc(N3CCCCC3)ccc2C2=CC3=CC(c4ccc(N5CCCCC5)cc4Nc4ccc(C)o4)=NC4=NC(c5cc(C(F)(F)F)nc(C(F)(F)F)c5)=NC(=N2)N34)o1 ; Cc1coc(C2=NC3=NC(c4ccc(N5CCCCC5)cc4Nc4ccc(C)o4)=CC4=CC(c5ccc(N6CCCCC6)cc5Nc5ccc(C)o5)=NC(=N2)N43)n1 ; Cc1ccc(Nc2cc(N3CCCCC3)ccc2C2=CC3=CC(c4ccc(N5CCCCC5)cc4Nc4ccc(C)o4)=NC4=NC(c5nc(F)co5)=NC(=N2)N34)o1]

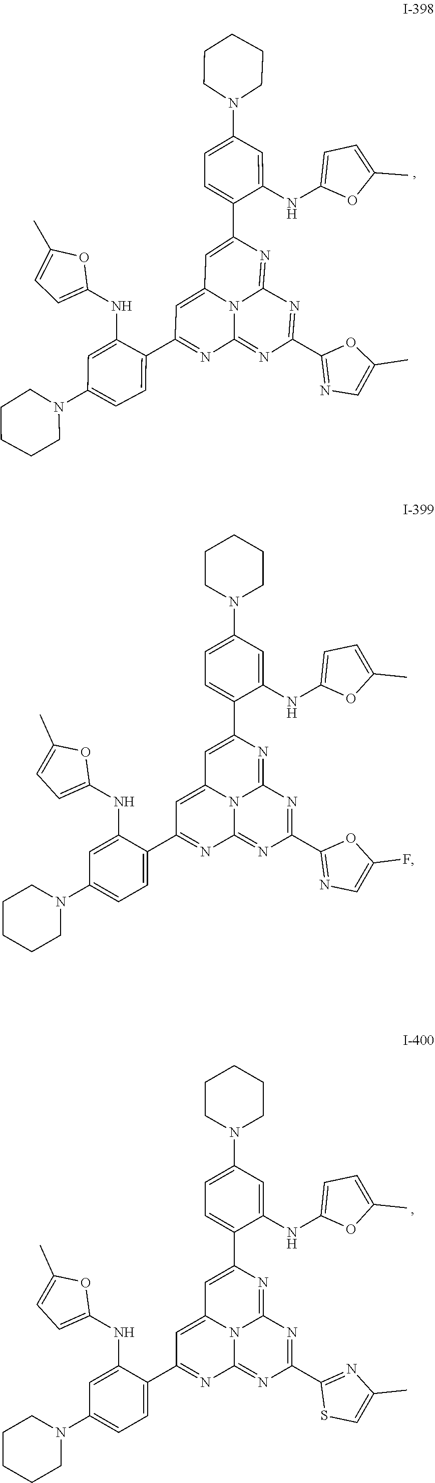 [SMILES: Cc1ccc(Nc2cc(N3CCCCC3)ccc2C2=CC3=CC(c4ccc(N5CCCCC5)cc4Nc4ccc(C)o4)=NC4=NC(c5ncc(C)o5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ncc(F)o5)=NC(=N2)N34)o1 ; Cc1csc(C2=NC3=NC(c4ccc(N5CCCCC5)cc4Nc4ccc(C)o4)=CC4=CC(c5ccc(N6CCCCC6)cc5Nc5ccc(C)o5)=NC(=N2)N43)n1]

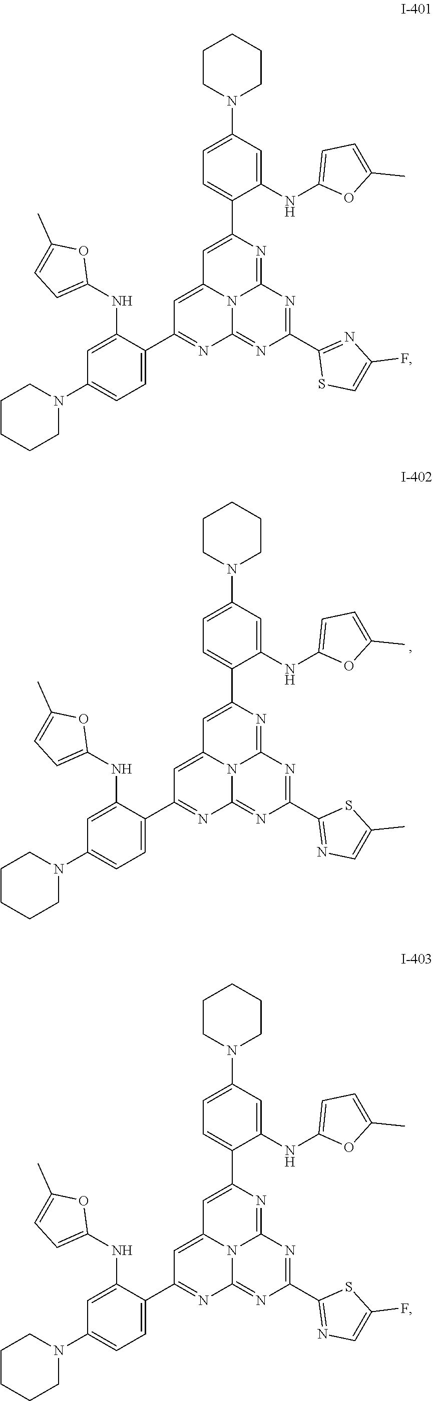 [SMILES: Cc1ccc(Nc2cc(N3CCCCC3)ccc2C2=CC3=CC(c4ccc(N5CCCCC5)cc4Nc4ccc(C)o4)=NC4=NC(c5nc(F)cs5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ncc(C)s5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ncc(F)s5)=NC(=N2)N34)o1]

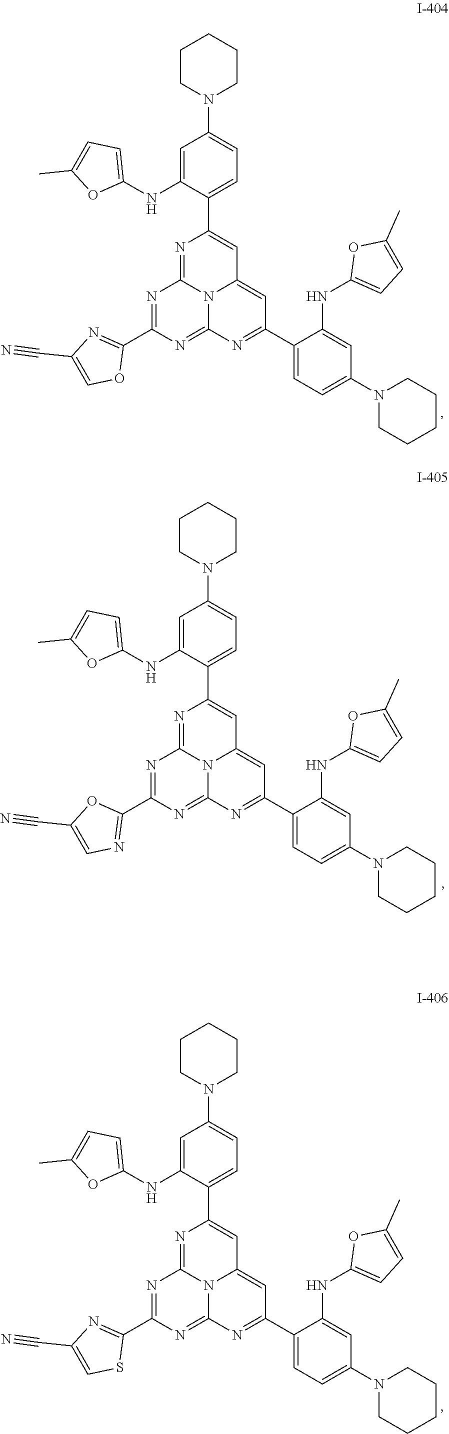 [SMILES: Cc1ccc(Nc2cc(N3CCCCC3)ccc2C2=CC3=CC(c4ccc(N5CCCCC5)cc4Nc4ccc(C)o4)=NC4=NC(c5nc(C#N)co5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ncc(C#N)o5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5nc(C#N)cs5)=NC(=N2)N34)o1]

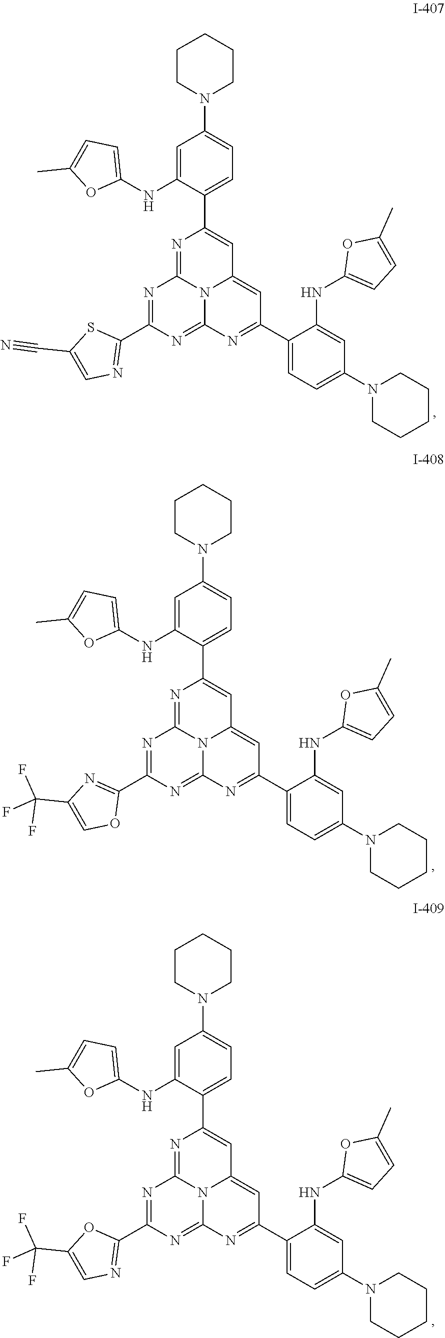 [SMILES: Cc1ccc(Nc2cc(N3CCCCC3)ccc2C2=CC3=CC(c4ccc(N5CCCCC5)cc4Nc4ccc(C)o4)=NC4=NC(c5ncc(C#N)s5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5nc(C(F)(F)F)co5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ncc(C(F)(F)F)o5)=NC(=N2)N34)o1]

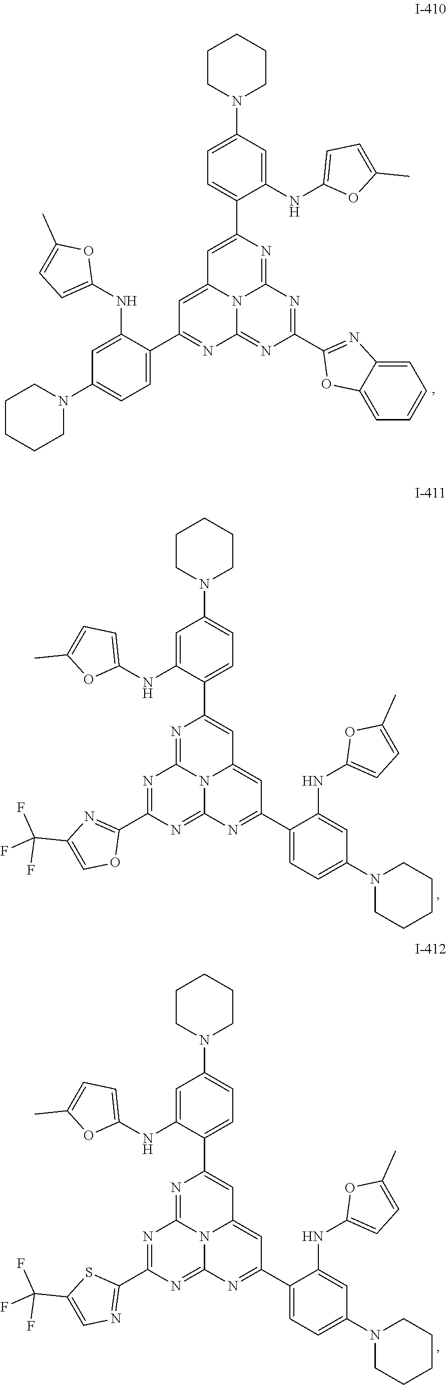 [SMILES: Cc1ccc(Nc2cc(N3CCCCC3)ccc2C2=CC3=CC(c4ccc(N5CCCCC5)cc4Nc4ccc(C)o4)=NC4=NC(c5nc6ccccc6o5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5nc(C(F)(F)F)co5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ncc(C(F)(F)F)s5)=NC(=N2)N34)o1]

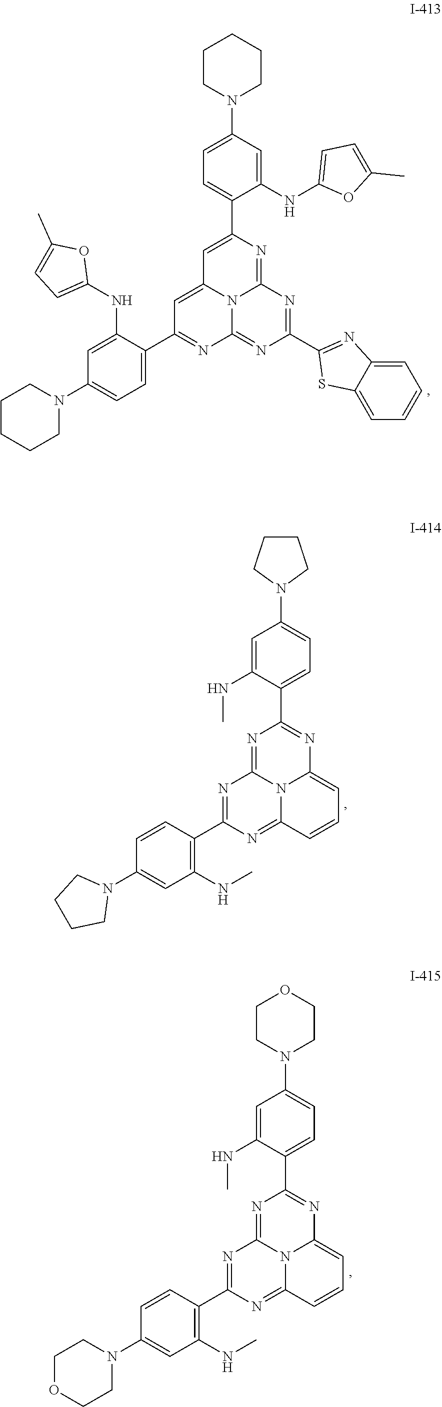 [SMILES: Cc1ccc(Nc2cc(N3CCCCC3)ccc2C2=CC3=CC(c4ccc(N5CCCCC5)cc4Nc4ccc(C)o4)=NC4=NC(c5nc6ccccc6s5)=NC(=N2)N34)o1 ; CNc1cc(N2CCCC2)ccc1C1=NC2=CC=CC3=NC(c4ccc(N5CCCC5)cc4NC)=NC(=N1)N23 ; CNc1cc(N2CCOCC2)ccc1C1=NC2=CC=CC3=NC(c4ccc(N5CCOCC5)cc4NC)=NC(=N1)N23]

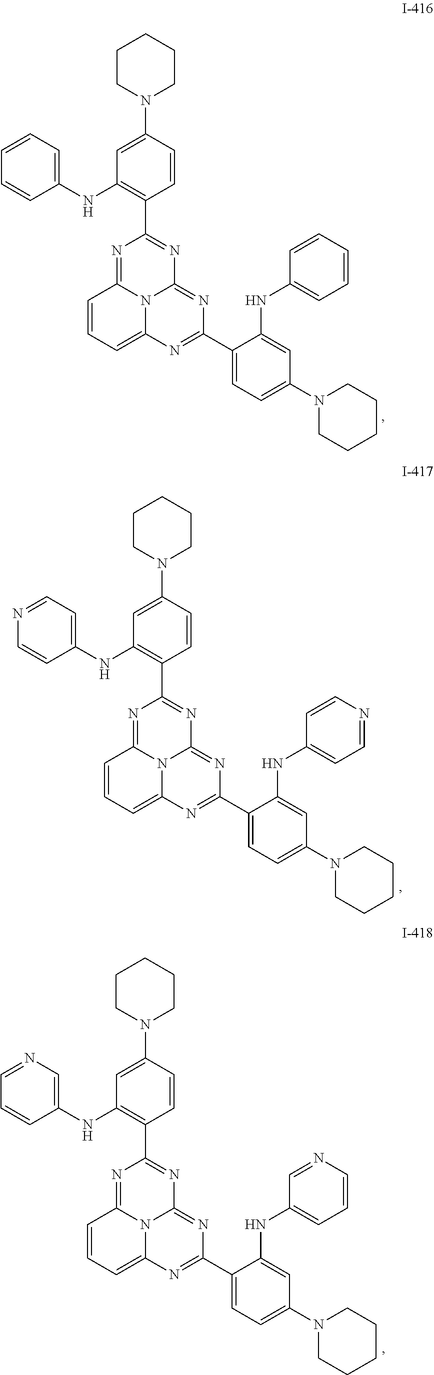 [SMILES: C1=CC2=NC(c3ccc(N4CCCCC4)cc3Nc3ccccc3)=NC3=NC(c4ccc(N5CCCCC5)cc4Nc4ccccc4)=NC(=C1)N23 ; C1=CC2=NC(c3ccc(N4CCCCC4)cc3Nc3ccncc3)=NC3=NC(c4ccc(N5CCCCC5)cc4Nc4ccncc4)=NC(=C1)N23 ; C1=CC2=NC(c3ccc(N4CCCCC4)cc3Nc3cccnc3)=NC3=NC(c4ccc(N5CCCCC5)cc4Nc4cccnc4)=NC(=C1)N23]

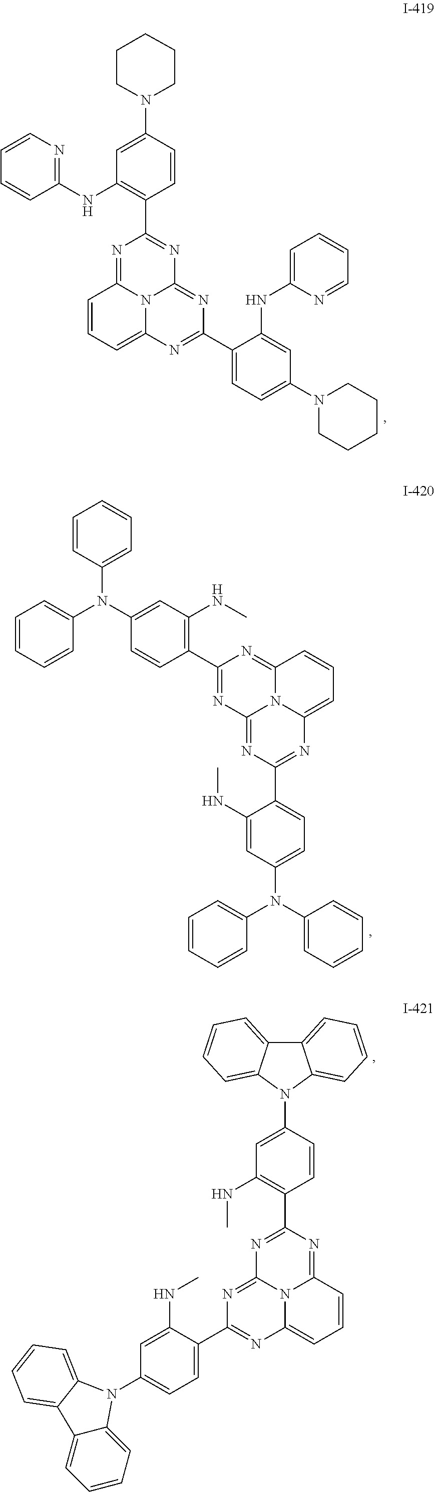 [SMILES: C1=CC2=NC(c3ccc(N4CCCCC4)cc3Nc3ccccn3)=NC3=NC(c4ccc(N5CCCCC5)cc4Nc4ccccn4)=NC(=C1)N23 ; CNc1cc(N(c2ccccc2)c2ccccc2)ccc1C1=NC2=CC=CC3=NC(c4ccc(N(c5ccccc5)c5ccccc5)cc4NC)=NC(=N1)N23 ; CNc1cc(-n2c3ccccc3c3ccccc32)ccc1C1=NC2=CC=CC3=NC(c4ccc(-n5c6ccccc6c6ccccc65)cc4NC)=NC(=N1)N23]

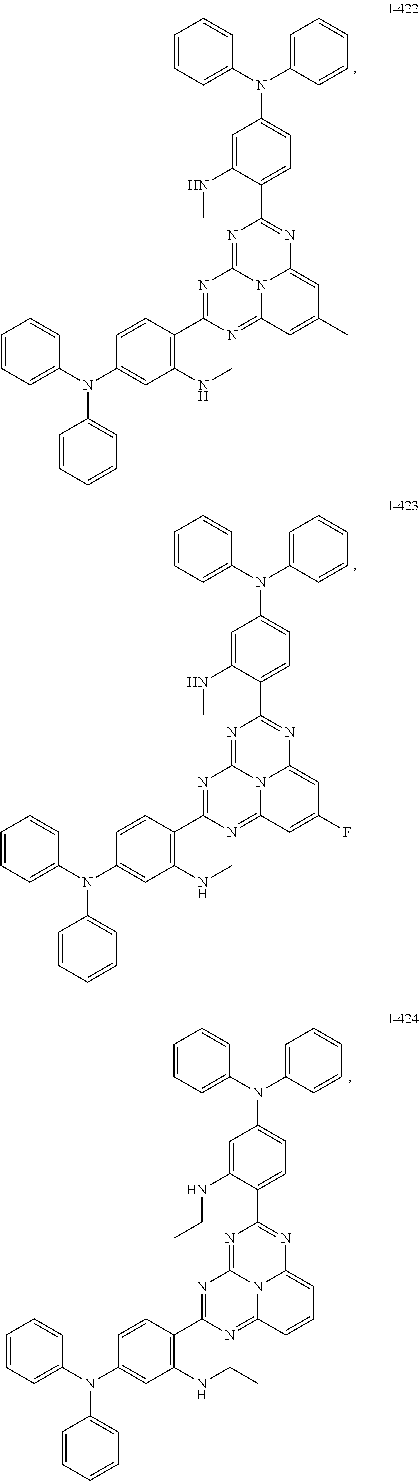 [SMILES: CNc1cc(N(c2ccccc2)c2ccccc2)ccc1C1=NC2=CC(C)=CC3=NC(c4ccc(N(c5ccccc5)c5ccccc5)cc4NC)=NC(=N1)N23 ; CNc1cc(N(c2ccccc2)c2ccccc2)ccc1C1=NC2=CC(F)=CC3=NC(c4ccc(N(c5ccccc5)c5ccccc5)cc4NC)=NC(=N1)N23 ; CCNc1cc(N(c2ccccc2)c2ccccc2)ccc1C1=NC2=CC=CC3=NC(c4ccc(N(c5ccccc5)c5ccccc5)cc4NCC)=NC(=N1)N23]

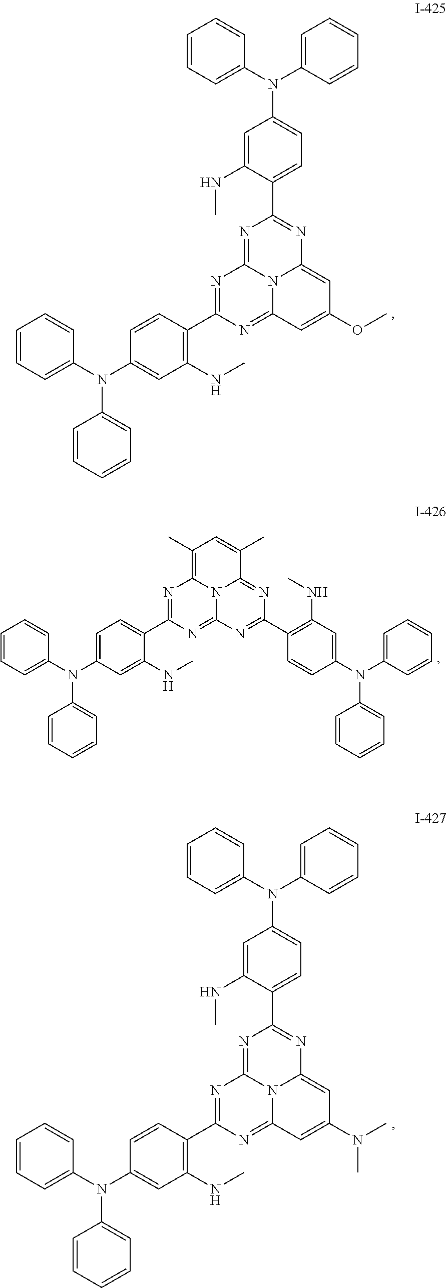 [SMILES: CNc1cc(N(c2ccccc2)c2ccccc2)ccc1C1=NC2=CC(OC)=CC3=NC(c4ccc(N(c5ccccc5)c5ccccc5)cc4NC)=NC(=N1)N23 ; CNc1cc(N(c2ccccc2)c2ccccc2)ccc1C1=NC2=NC(c3ccc(N(c4ccccc4)c4ccccc4)cc3NC)=NC3=C(C)C=C(C)C(=N1)N23 ; CNc1cc(N(c2ccccc2)c2ccccc2)ccc1C1=NC2=CC(N(C)C)=CC3=NC(c4ccc(N(c5ccccc5)c5ccccc5)cc4NC)=NC(=N1)N23]

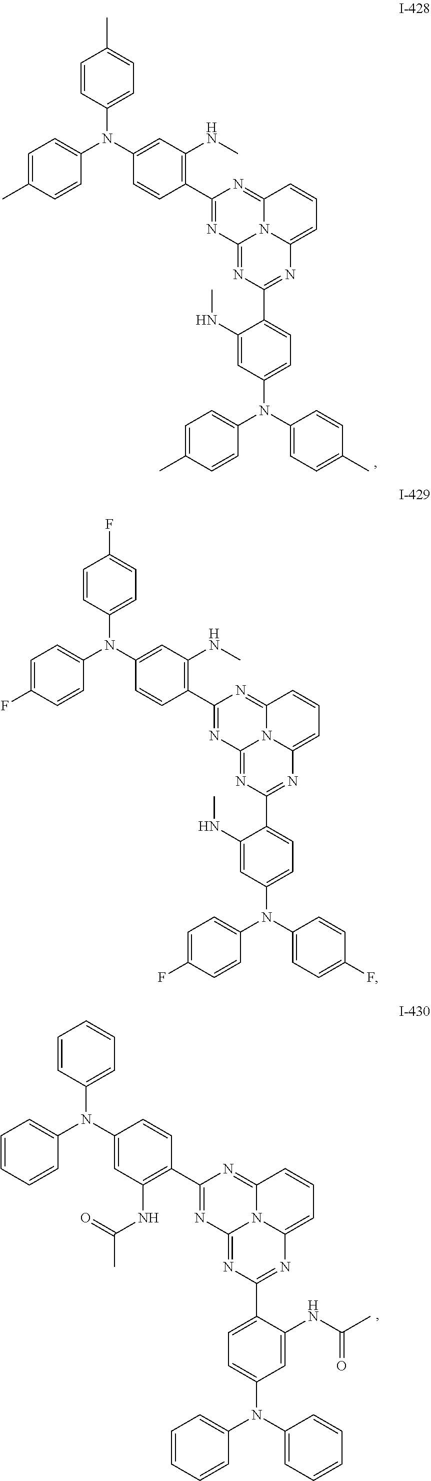

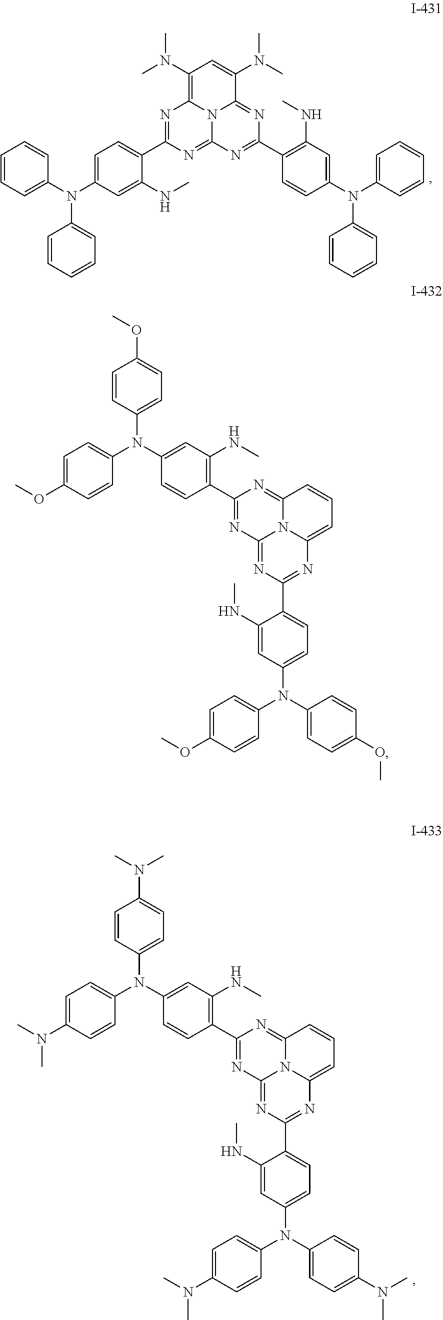

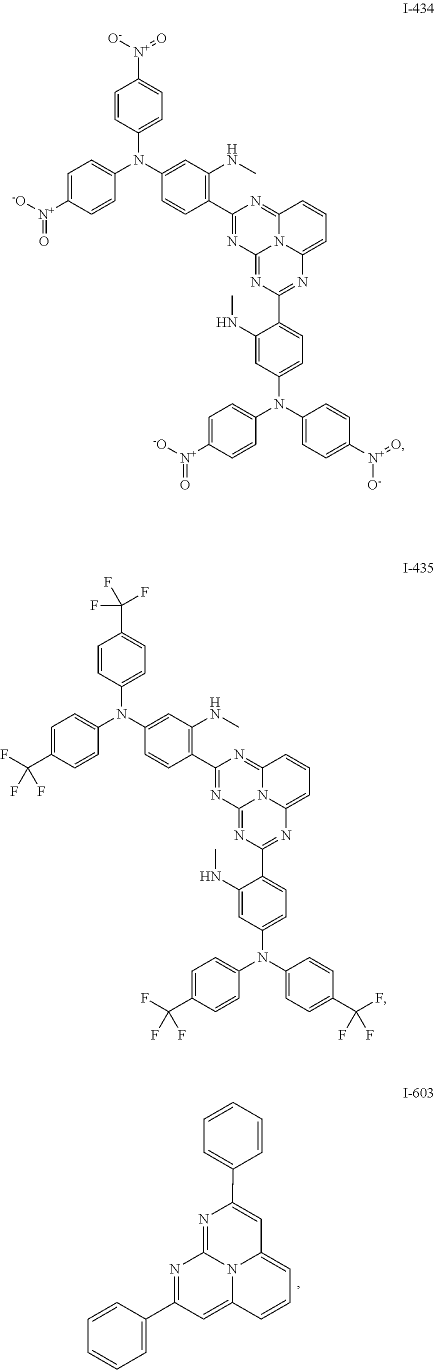 [SMILES: CNc1cc(N(c2ccc([N+](=O)[O-])cc2)c2ccc([N+](=O)[O-])cc2)ccc1C1=NC2=CC=CC3=NC(c4ccc(N(c5ccc([N+](=O)[O-])cc5)c5ccc([N+](=O)[O-])cc5)cc4NC)=NC(=N1)N23 ; CNc1cc(N(c2ccc(C(F)(F)F)cc2)c2ccc(C(F)(F)F)cc2)ccc1C1=NC2=CC=CC3=NC(c4ccc(N(c5ccc(C(F)(F)F)cc5)c5ccc(C(F)(F)F)cc5)cc4NC)=NC(=N1)N23 ; C1=CC2=CC(c3ccccc3)=NC3=NC(c4ccccc4)=CC(=C1)N23]

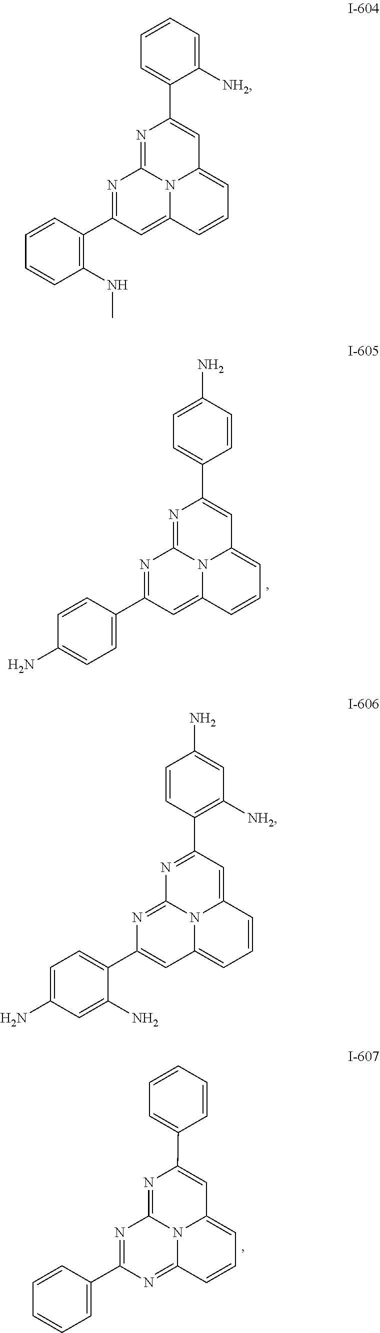 [SMILES: CNc1ccccc1C1=NC2=NC(c3ccccc3N)=CC3=CC=CC(=C1)N32 ; Nc1ccc(C2=CC3=CC=CC4=CC(c5ccc(N)cc5)=NC(=N2)N34)cc1 ; Nc1ccc(C2=CC3=CC=CC4=CC(c5ccc(N)cc5N)=NC(=N2)N34)c(N)c1 ; C1=CC2=NC(c3ccccc3)=NC3=NC(c4ccccc4)=CC(=C1)N23]

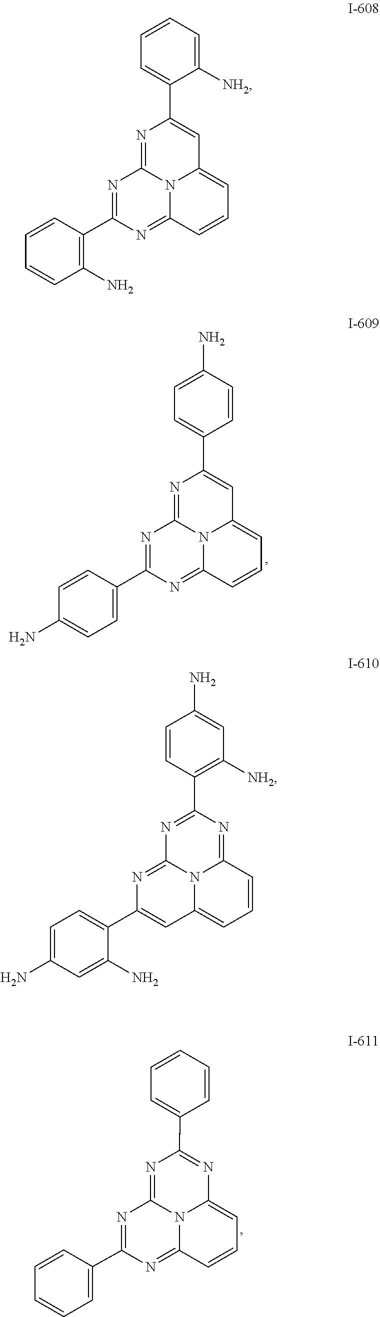 [SMILES: Nc1ccccc1C1=CC2=CC=CC3=NC(c4ccccc4N)=NC(=N1)N23 ; Nc1ccc(C2=CC3=CC=CC4=NC(c5ccc(N)cc5)=NC(=N2)N34)cc1 ; Nc1ccc(C2=CC3=CC=CC4=NC(c5ccc(N)cc5N)=NC(=N2)N34)c(N)c1 ; C1=CC2=NC(c3ccccc3)=NC3=NC(c4ccccc4)=NC(=C1)N23]

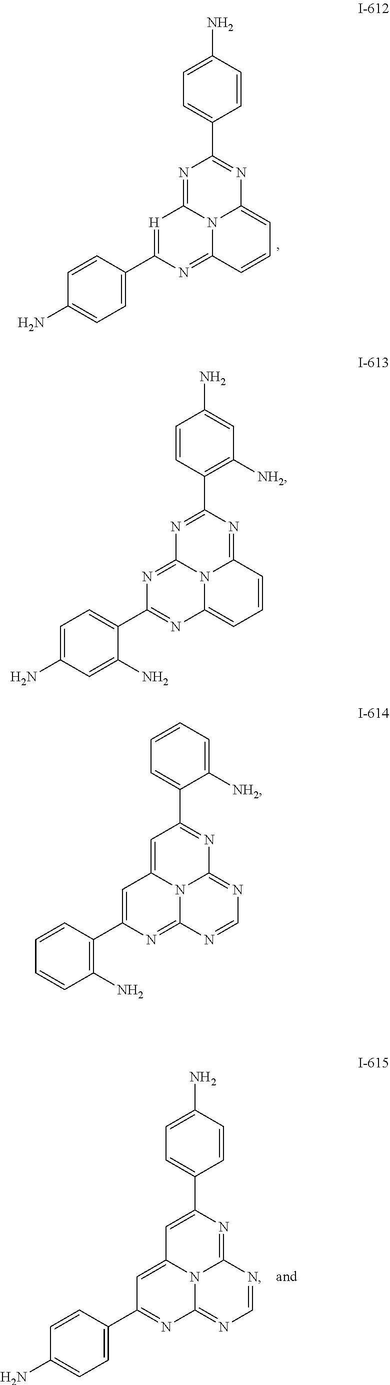

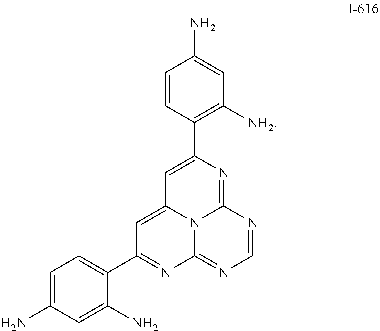 [SMILES: Nc1ccc(C2=CC3=CC(c4ccc(N)cc4N)=NC4=NC=NC(=N2)N34)c(N)c1]

In some embodiments, the compound has a structure of Formula I-a

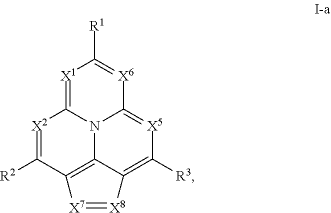

wherein
X⁷ is selected from N and CR¹¹; and
X⁸ is selected from N and CR¹².

In some embodiments, R¹¹ and R¹² are each independently selected from H, NH₂, NH(alkyl), NH(aryl), and NH-heteroaryl. In some embodiments, R¹¹ and R¹² are H or NH₂.

In some embodiment, the compound is selected from

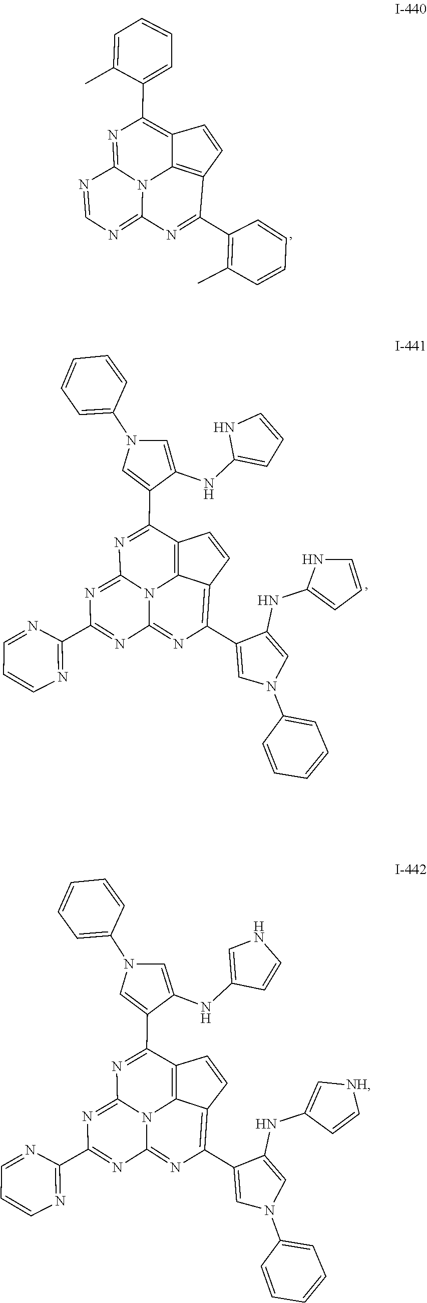 [SMILES: Cc1ccccc1-c1nc2ncnc3nc(-c4ccccc4C)c4ccc1c4n23 ; c1ccc(-n2cc(Nc3ccc[nH]3)c(-c3nc4nc(-c5ncccn5)nc5nc(-c6cn(-c7ccccc7)cc6Nc6ccc[nH]6)c6ccc3c6n45)c2)cc1 ; c1ccc(-n2cc(Nc3cc[nH]c3)c(-c3nc4nc(-c5ncccn5)nc5nc(-c6cn(-c7ccccc7)cc6Nc6cc[nH]c6)c6ccc3c6n45)c2)cc1]

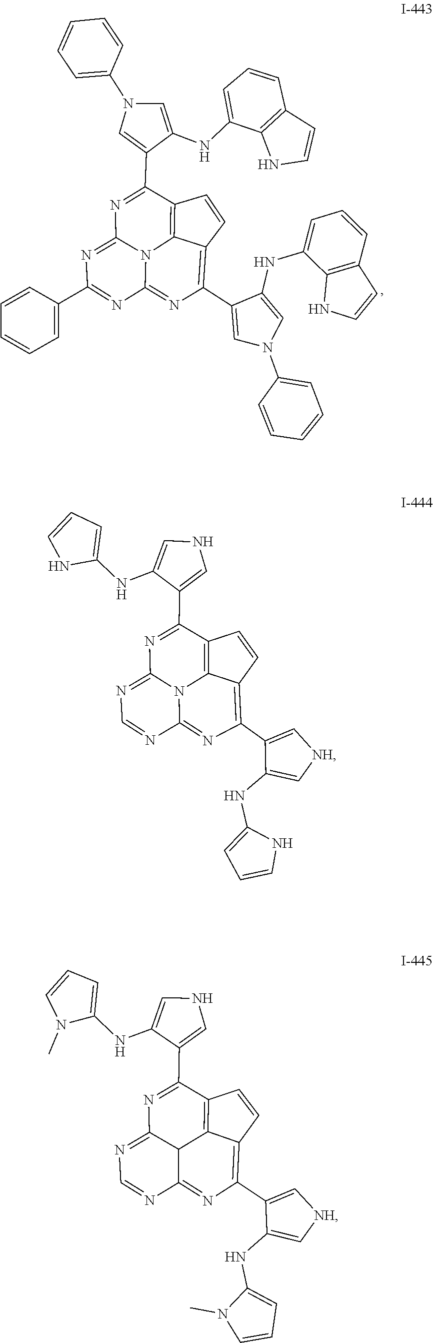 [SMILES: c1ccc(-c2nc3nc(-c4cn(-c5ccccc5)cc4Nc4cccc5cc[nH]c45)c4ccc5c(-c6cn(-c7ccccc7)cc6Nc6cccc7cc[nH]c67)nc(n2)n3c45)cc1 ; c1c[nH]c(Nc2c[nH]cc2-c2nc3ncnc4nc(-c5c[nH]cc5Nc5ccc[nH]5)c5ccc2c5n34)c1 ; Cn1cccc1Nc1c[nH]cc1C1=NC2=NC=NC3=NC(c4c[nH]cc4Nc4cccn4C)=C4C=CC1=C4C23]

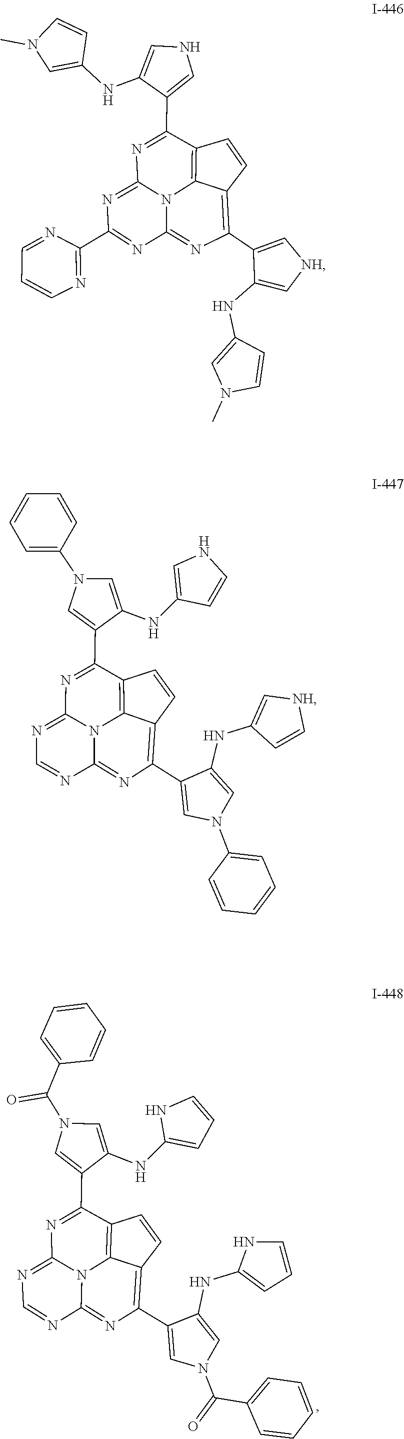 [SMILES: Cn1ccc(Nc2c[nH]cc2-c2nc3nc(-c4ncccn4)nc4nc(-c5c[nH]cc5Nc5ccn(C)c5)c5ccc2c5n34)c1 ; c1ccc(-n2cc(Nc3cc[nH]c3)c(-c3nc4ncnc5nc(-c6cn(-c7ccccc7)cc6Nc6cc[nH]c6)c6ccc3c6n45)c2)cc1 ; O=C(c1ccccc1)n1cc(Nc2ccc[nH]2)c(-c2nc3ncnc4nc(-c5cn(C(=O)c6ccccc6)cc5Nc5ccc[nH]5)c5ccc2c5n34)c1]

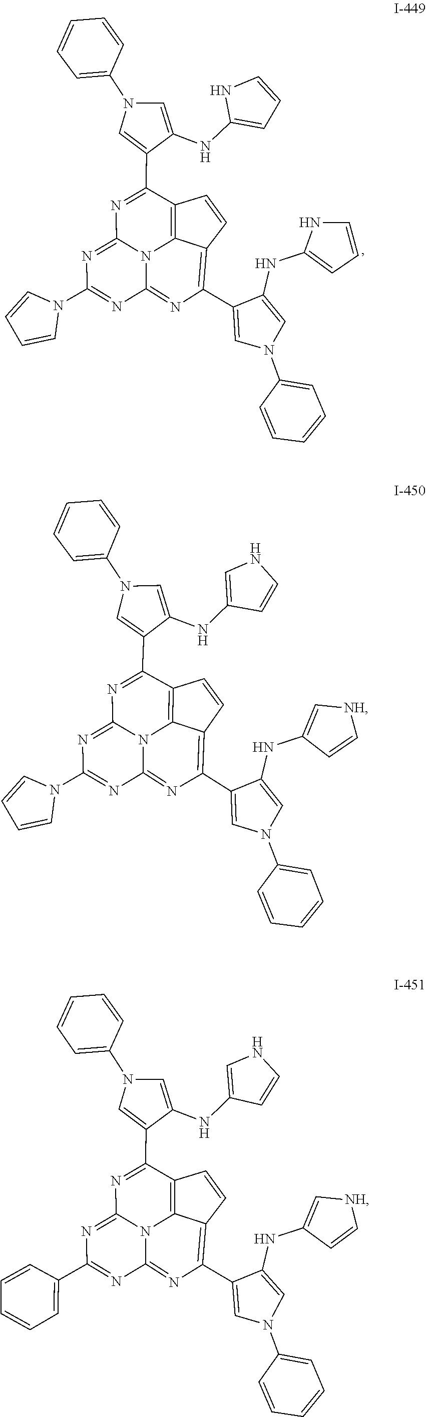 [SMILES: c1ccc(-n2cc(Nc3ccc[nH]3)c(-c3nc4nc(-n5cccc5)nc5nc(-c6cn(-c7ccccc7)cc6Nc6ccc[nH]6)c6ccc3c6n45)c2)cc1 ; c1ccc(-n2cc(Nc3cc[nH]c3)c(-c3nc4nc(-n5cccc5)nc5nc(-c6cn(-c7ccccc7)cc6Nc6cc[nH]c6)c6ccc3c6n45)c2)cc1 ; c1ccc(-c2nc3nc(-c4cn(-c5ccccc5)cc4Nc4cc[nH]c4)c4ccc5c(-c6cn(-c7ccccc7)cc6Nc6cc[nH]c6)nc(n2)n3c45)cc1]

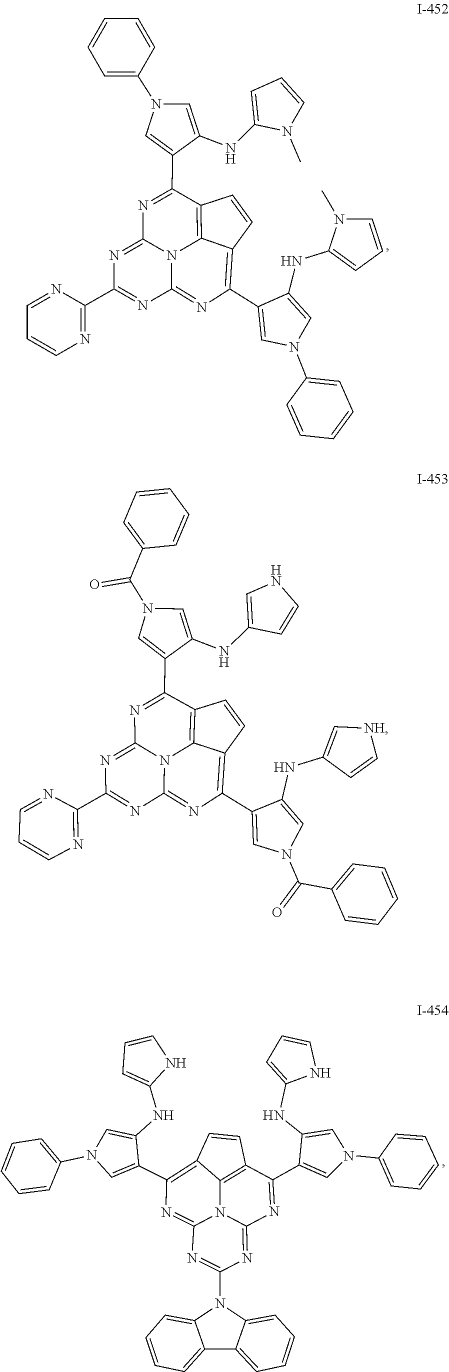 [SMILES: Cn1cccc1Nc1cn(-c2ccccc2)cc1-c1nc2nc(-c3ncccn3)nc3nc(-c4cn(-c5ccccc5)cc4Nc4cccn4C)c4ccc1c4n23 ; O=C(c1ccccc1)n1cc(Nc2cc[nH]c2)c(-c2nc3nc(-c4ncccn4)nc4nc(-c5cn(C(=O)c6ccccc6)cc5Nc5cc[nH]c5)c5ccc2c5n34)c1 ; c1ccc(-n2cc(Nc3ccc[nH]3)c(-c3nc4nc(-n5c6ccccc6c6ccccc65)nc5nc(-c6cn(-c7ccccc7)cc6Nc6ccc[nH]6)c6ccc3c6n45)c2)cc1]

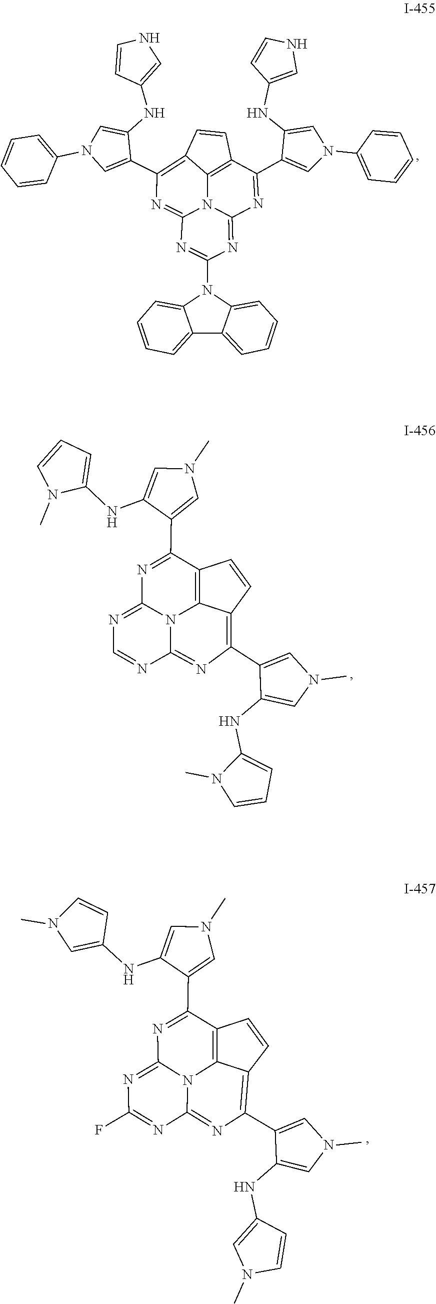

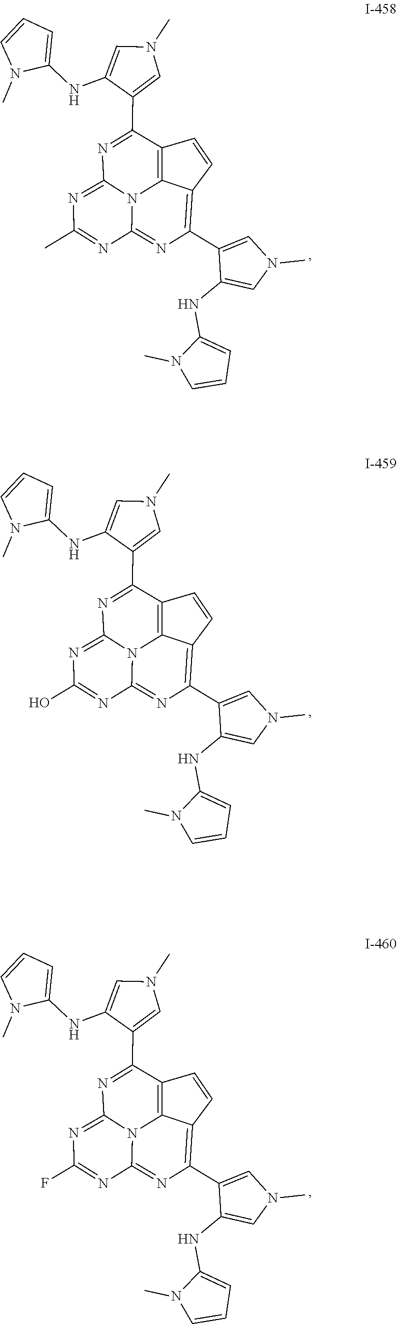 [SMILES: Cc1nc2nc(-c3cn(C)cc3Nc3cccn3C)c3ccc4c(-c5cn(C)cc5Nc5cccn5C)nc(n1)n2c34 ; Cn1cc(Nc2cccn2C)c(-c2nc3nc(O)nc4nc(-c5cn(C)cc5Nc5cccn5C)c5ccc2c5n34)c1 ; Cn1cc(Nc2cccn2C)c(-c2nc3nc(F)nc4nc(-c5cn(C)cc5Nc5cccn5C)c5ccc2c5n34)c1]

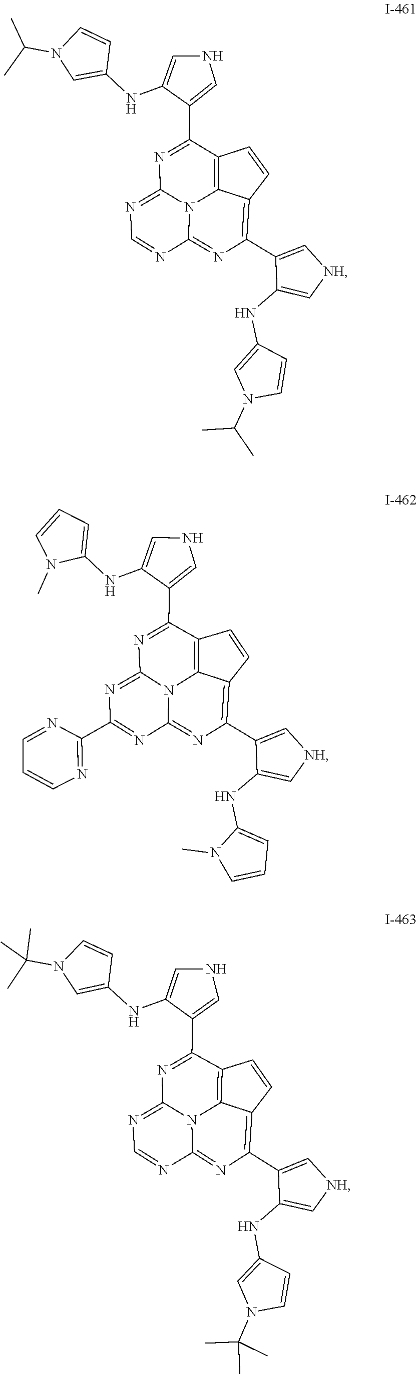 [SMILES: CC(C)n1ccc(Nc2c[nH]cc2-c2nc3ncnc4nc(-c5c[nH]cc5Nc5ccn(C(C)C)c5)c5ccc2c5n34)c1 ; Cn1cccc1Nc1c[nH]cc1-c1nc2nc(-c3ncccn3)nc3nc(-c4c[nH]cc4Nc4cccn4C)c4ccc1c4n23 ; CC(C)(C)n1ccc(Nc2c[nH]cc2-c2nc3ncnc4nc(-c5c[nH]cc5Nc5ccn(C(C)(C)C)c5)c5ccc2c5n34)c1]

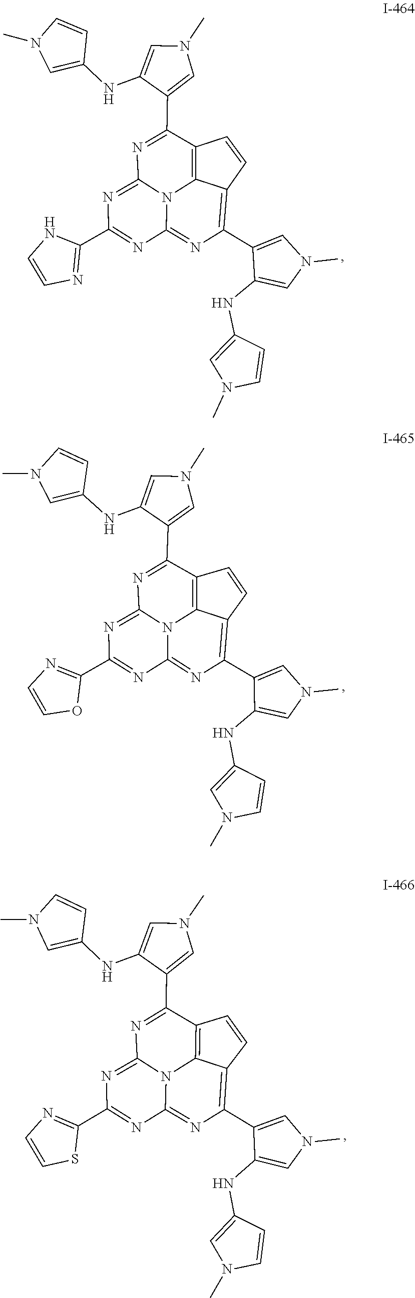

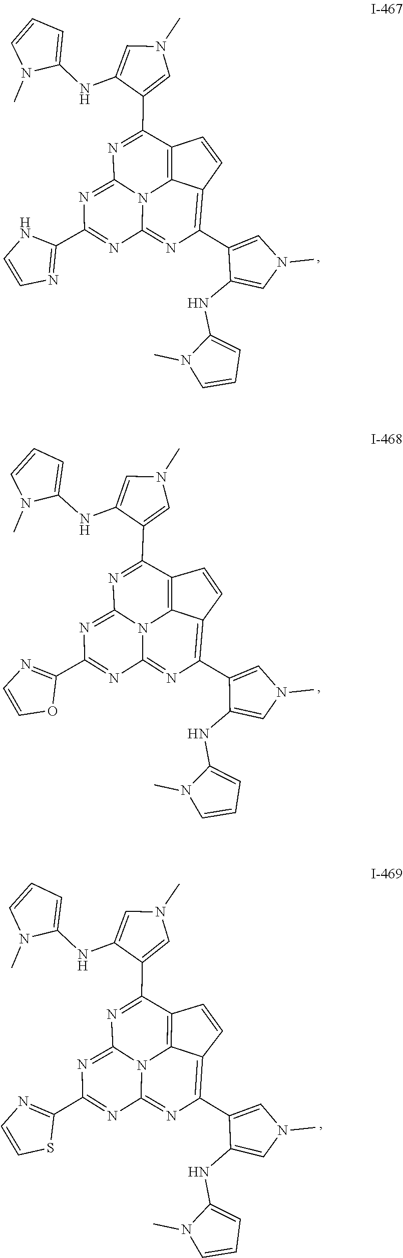 [SMILES: Cn1cc(Nc2cccn2C)c(-c2nc3nc(-c4ncc[nH]4)nc4nc(-c5cn(C)cc5Nc5cccn5C)c5ccc2c5n34)c1 ; Cn1cc(Nc2cccn2C)c(-c2nc3nc(-c4ncco4)nc4nc(-c5cn(C)cc5Nc5cccn5C)c5ccc2c5n34)c1 ; Cn1cc(Nc2cccn2C)c(-c2nc3nc(-c4nccs4)nc4nc(-c5cn(C)cc5Nc5cccn5C)c5ccc2c5n34)c1]

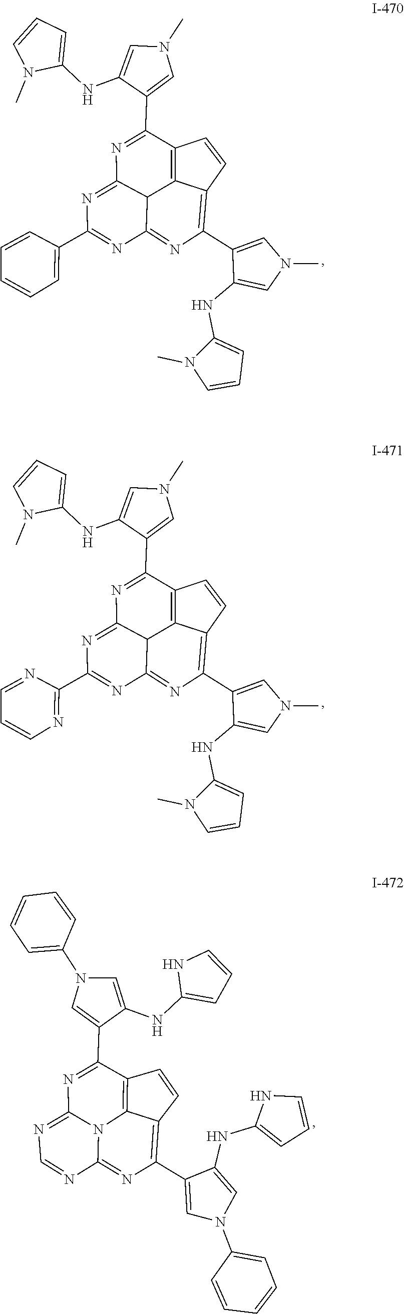 [SMILES: Cn1cc(Nc2cccn2C)c(C2=NC3=NC(c4ccccc4)=NC4=NC(c5cn(C)cc5Nc5cccn5C)=C5C=CC2=C5C34)c1 ; Cn1cc(Nc2cccn2C)c(C2=NC3=NC(c4ncccn4)=NC4=NC(c5cn(C)cc5Nc5cccn5C)=C5C=CC2=C5C34)c1 ; c1ccc(-n2cc(Nc3ccc[nH]3)c(-c3nc4ncnc5nc(-c6cn(-c7ccccc7)cc6Nc6ccc[nH]6)c6ccc3c6n45)c2)cc1]

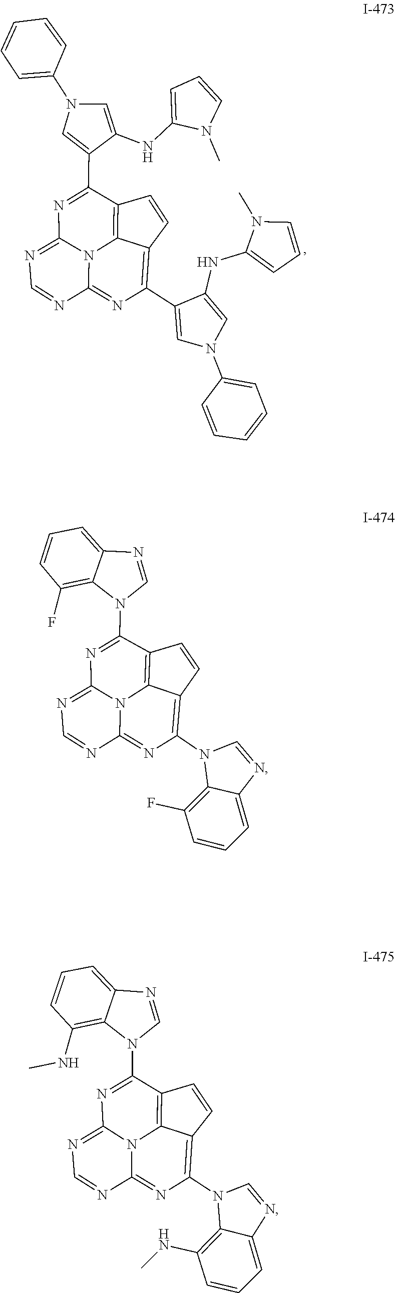 [SMILES: Cn1cccc1Nc1cn(-c2ccccc2)cc1-c1nc2ncnc3nc(-c4cn(-c5ccccc5)cc4Nc4cccn4C)c4ccc1c4n23 ; Fc1cccc2ncn(-c3nc4ncnc5nc(-n6cnc7cccc(F)c76)c6ccc3c6n45)c12 ; CNc1cccc2ncn(-c3nc4ncnc5nc(-n6cnc7cccc(NC)c76)c6ccc3c6n45)c12]

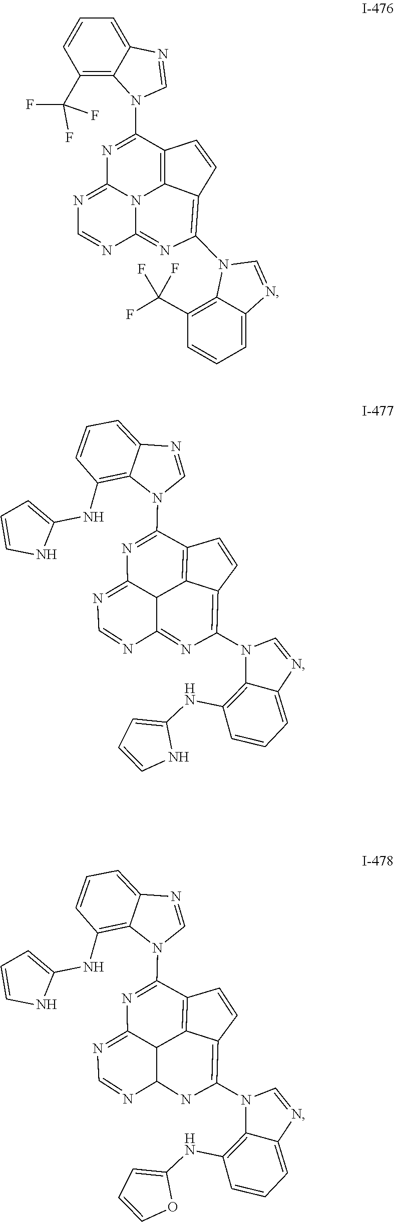 [SMILES: FC(F)(F)c1cccc2ncn(-c3nc4ncnc5nc(-n6cnc7cccc(C(F)(F)F)c76)c6ccc3c6n45)c12 ; C1=CC2=C(n3cnc4cccc(Nc5ccc[nH]5)c43)N=C3N=CN=C4N=C(n5cnc6cccc(Nc7ccc[nH]7)c65)C1=C2C43 ; C1=CC2=C(n3cnc4cccc(Nc5ccco5)c43)NC3N=CN=C4N=C(n5cnc6cccc(Nc7ccc[nH]7)c65)C1=C2C43]

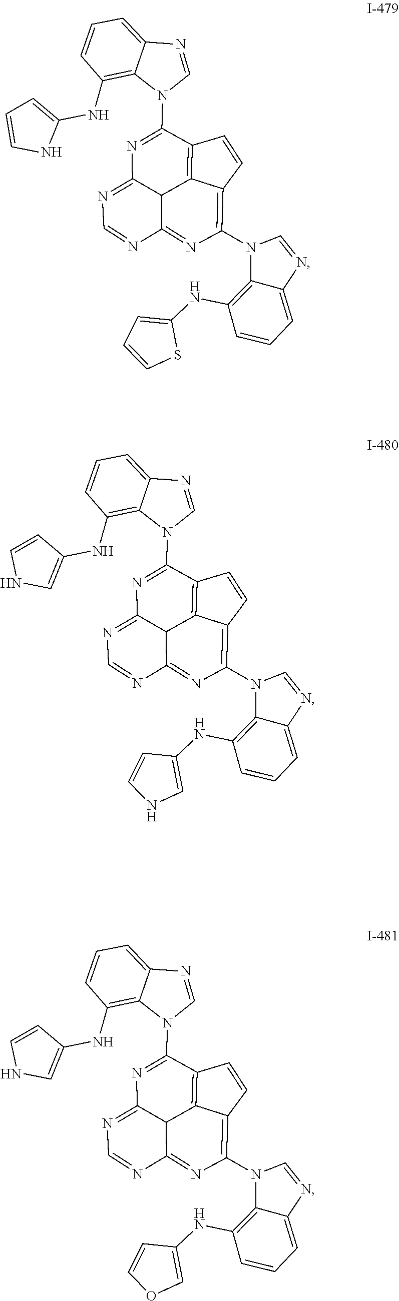 [SMILES: C1=CC2=C(n3cnc4cccc(Nc5cccs5)c43)N=C3N=CN=C4N=C(n5cnc6cccc(Nc7ccc[nH]7)c65)C1=C2C43 ; C1=CC2=C(n3cnc4cccc(Nc5cc[nH]c5)c43)N=C3N=CN=C4N=C(n5cnc6cccc(Nc7cc[nH]c7)c65)C1=C2C43 ; C1=CC2=C(n3cnc4cccc(Nc5ccoc5)c43)N=C3N=CN=C4N=C(n5cnc6cccc(Nc7cc[nH]c7)c65)C1=C2C43]

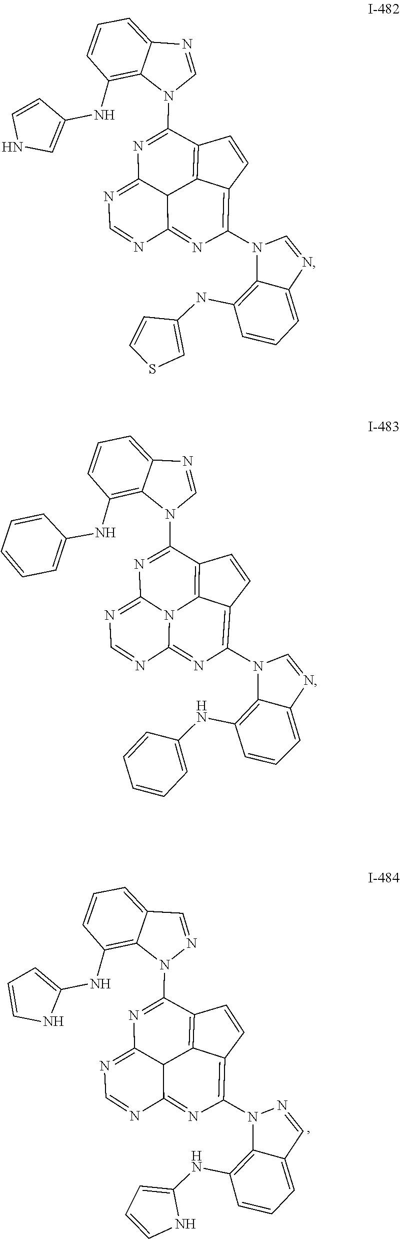

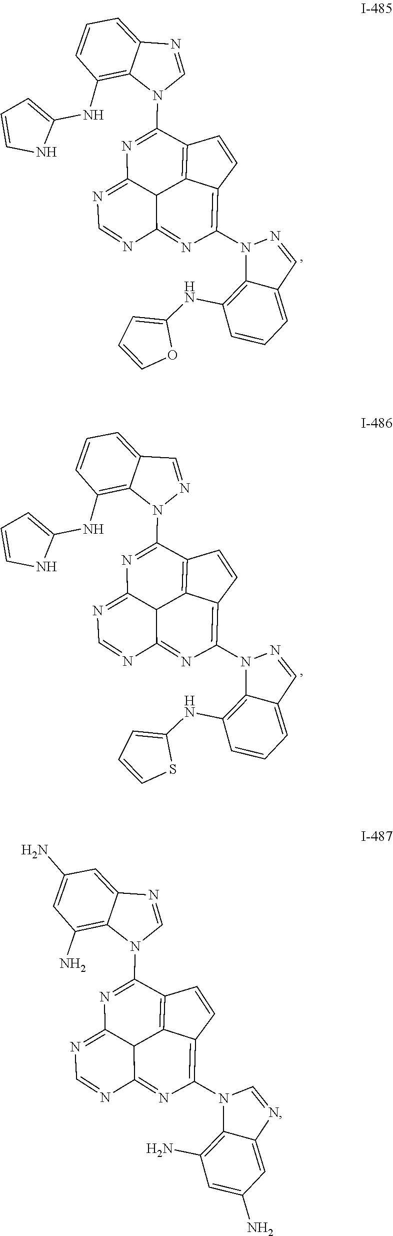 [SMILES: C1=CC2=C(n3ncc4cccc(Nc5ccco5)c43)N=C3N=CN=C4N=C(n5cnc6cccc(Nc7ccc[nH]7)c65)C1=C2C43 ; C1=CC2=C(n3ncc4cccc(Nc5cccs5)c43)N=C3N=CN=C4N=C(n5ncc6cccc(Nc7ccc[nH]7)c65)C1=C2C43 ; Nc1cc(N)c2c(c1)ncn2C1=NC2=NC=NC3=NC(n4cnc5cc(N)cc(N)c54)=C4C=CC1=C4C23]

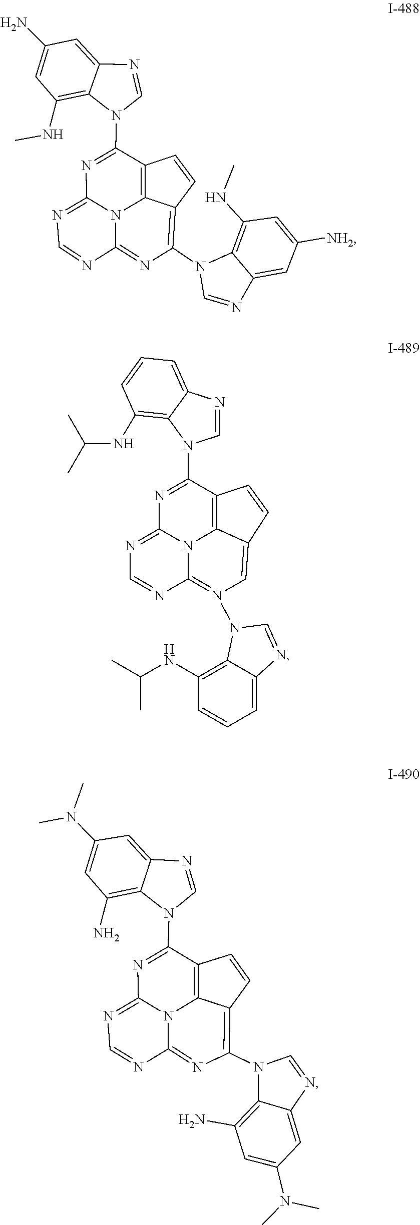

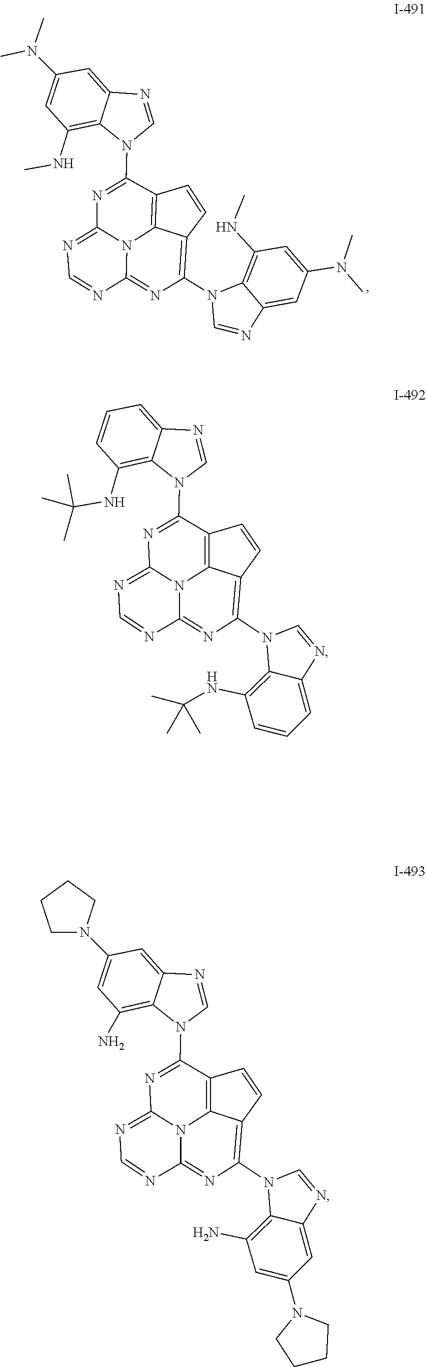

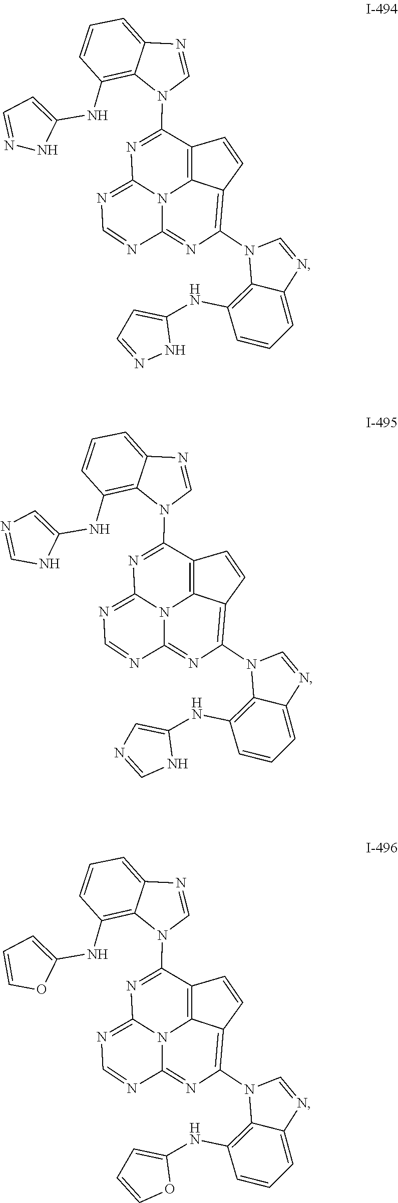 [SMILES: c1cc(Nc2ccn[nH]2)c2c(c1)ncn2-c1nc2ncnc3nc(-n4cnc5cccc(Nc6ccn[nH]6)c54)c4ccc1c4n23 ; c1cc(Nc2cnc[nH]2)c2c(c1)ncn2-c1nc2ncnc3nc(-n4cnc5cccc(Nc6cnc[nH]6)c54)c4ccc1c4n23 ; c1coc(Nc2cccc3ncn(-c4nc5ncnc6nc(-n7cnc8cccc(Nc9ccco9)c87)c7ccc4c7n56)c23)c1]

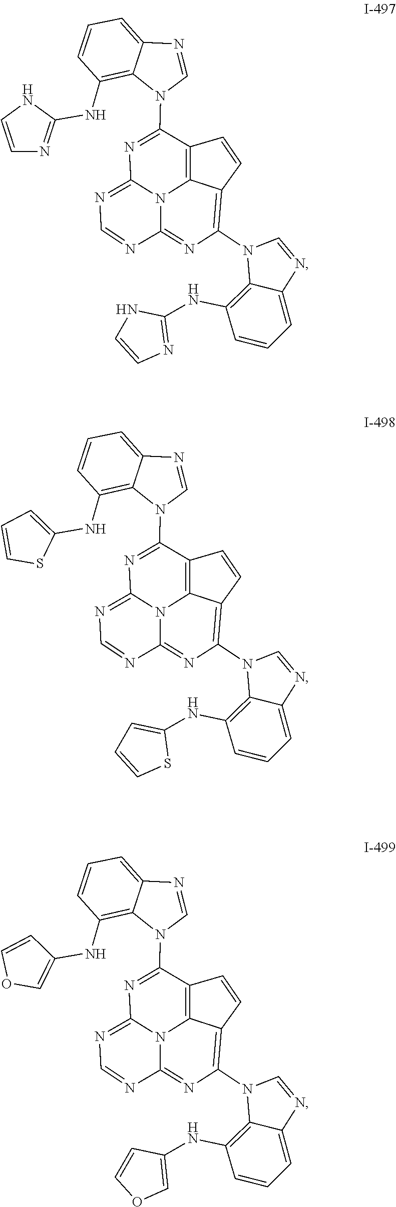 [SMILES: c1cc(Nc2ncc[nH]2)c2c(c1)ncn2-c1nc2ncnc3nc(-n4cnc5cccc(Nc6ncc[nH]6)c54)c4ccc1c4n23 ; c1csc(Nc2cccc3ncn(-c4nc5ncnc6nc(-n7cnc8cccc(Nc9cccs9)c87)c7ccc4c7n56)c23)c1 ; c1cc(Nc2ccoc2)c2c(c1)ncn2-c1nc2ncnc3nc(-n4cnc5cccc(Nc6ccoc6)c54)c4ccc1c4n23]

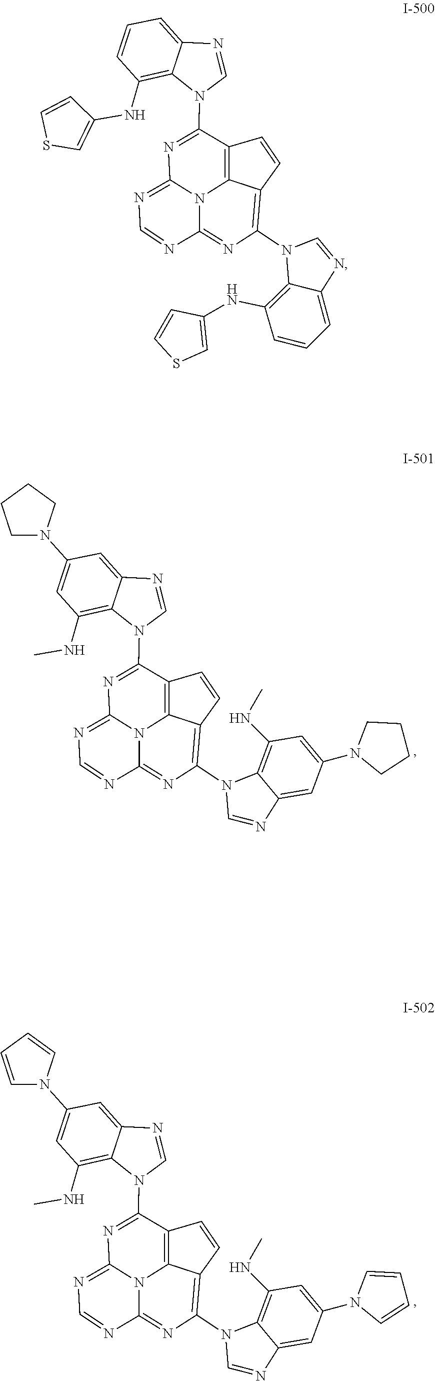 [SMILES: c1cc(Nc2ccsc2)c2c(c1)ncn2-c1nc2ncnc3nc(-n4cnc5cccc(Nc6ccsc6)c54)c4ccc1c4n23 ; CNc1cc(N2CCCC2)cc2ncn(-c3nc4ncnc5nc(-n6cnc7cc(N8CCCC8)cc(NC)c76)c6ccc3c6n45)c12 ; CNc1cc(-n2cccc2)cc2ncn(-c3nc4ncnc5nc(-n6cnc7cc(-n8cccc8)cc(NC)c76)c6ccc3c6n45)c12]

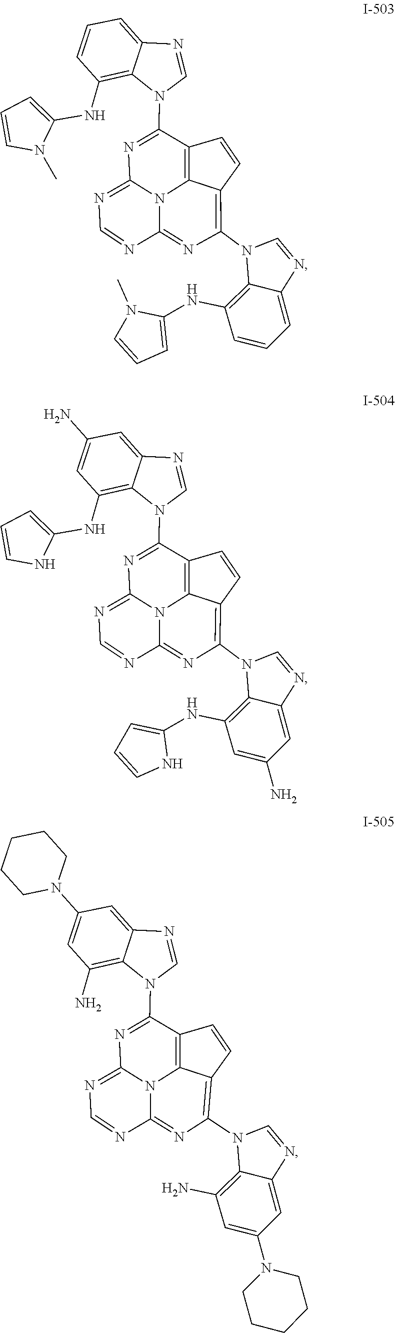 [SMILES: Cn1cccc1Nc1cccc2ncn(-c3nc4ncnc5nc(-n6cnc7cccc(Nc8cccn8C)c76)c6ccc3c6n45)c12 ; Nc1cc(Nc2ccc[nH]2)c2c(c1)ncn2-c1nc2ncnc3nc(-n4cnc5cc(N)cc(Nc6ccc[nH]6)c54)c4ccc1c4n23 ; Nc1cc(N2CCCCC2)cc2ncn(-c3nc4ncnc5nc(-n6cnc7cc(N8CCCCC8)cc(N)c76)c6ccc3c6n45)c12]

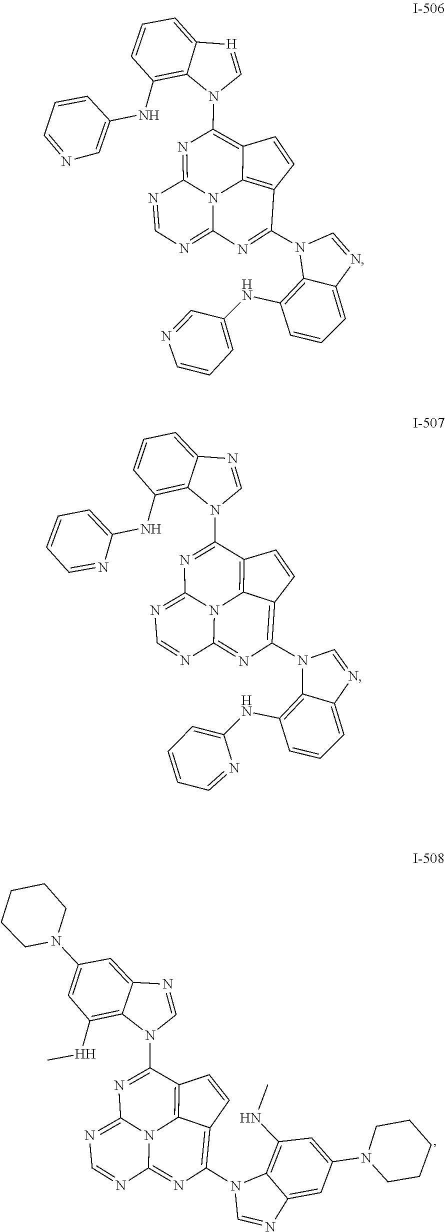

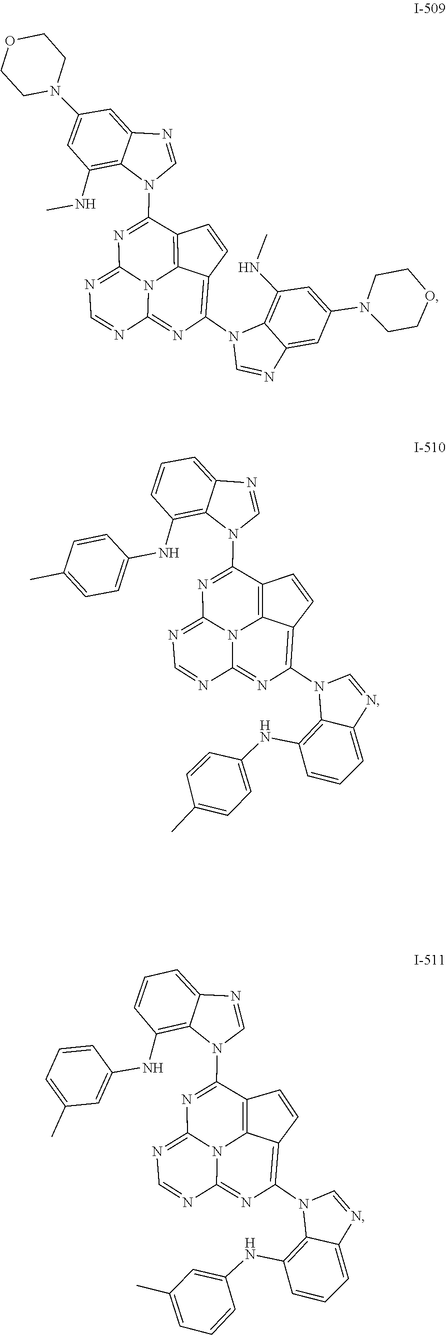 [SMILES: CNc1cc(N2CCOCC2)cc2ncn(-c3nc4ncnc5nc(-n6cnc7cc(N8CCOCC8)cc(NC)c76)c6ccc3c6n45)c12 ; Cc1ccc(Nc2cccc3ncn(-c4nc5ncnc6nc(-n7cnc8cccc(Nc9ccc(C)cc9)c87)c7ccc4c7n56)c23)cc1 ; Cc1cccc(Nc2cccc3ncn(-c4nc5ncnc6nc(-n7cnc8cccc(Nc9cccc(C)c9)c87)c7ccc4c7n56)c23)c1]

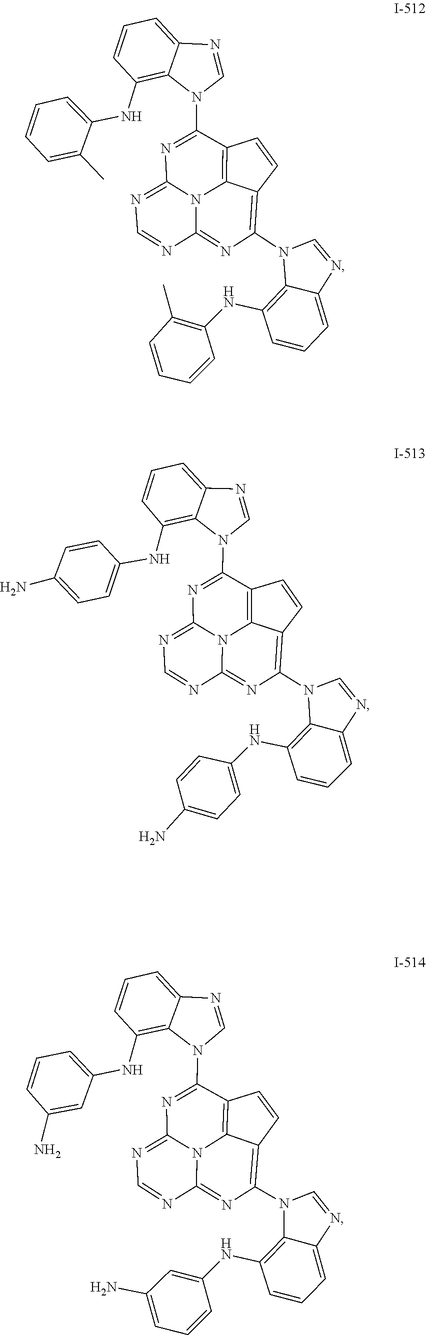 [SMILES: Cc1ccccc1Nc1cccc2ncn(-c3nc4ncnc5nc(-n6cnc7cccc(Nc8ccccc8C)c76)c6ccc3c6n45)c12 ; Nc1ccc(Nc2cccc3ncn(-c4nc5ncnc6nc(-n7cnc8cccc(Nc9ccc(N)cc9)c87)c7ccc4c7n56)c23)cc1 ; Nc1cccc(Nc2cccc3ncn(-c4nc5ncnc6nc(-n7cnc8cccc(Nc9cccc(N)c9)c87)c7ccc4c7n56)c23)c1]

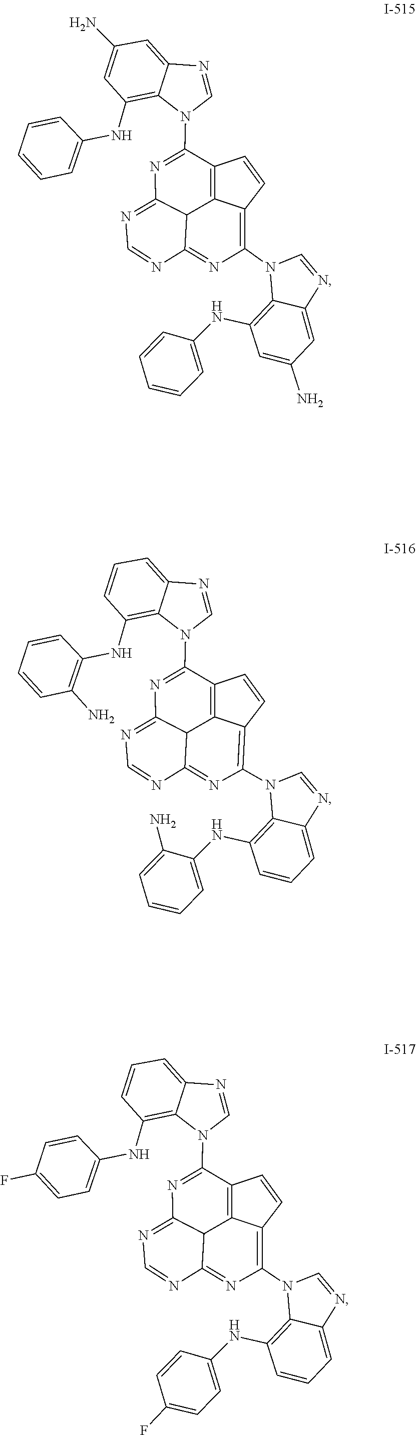 [SMILES: Nc1cc(Nc2ccccc2)c2c(c1)ncn2C1=NC2=NC=NC3=NC(n4cnc5cc(N)cc(Nc6ccccc6)c54)=C4C=CC1=C4C23 ; Nc1ccccc1Nc1cccc2ncn(C3=NC4=NC=NC5=NC(n6cnc7cccc(Nc8ccccc8N)c76)=C6C=CC3=C6C45)c12 ; Fc1ccc(Nc2cccc3ncn(C4=NC5=NC=NC6=NC(n7cnc8cccc(Nc9ccc(F)cc9)c87)=C7C=CC4=C7C56)c23)cc1]

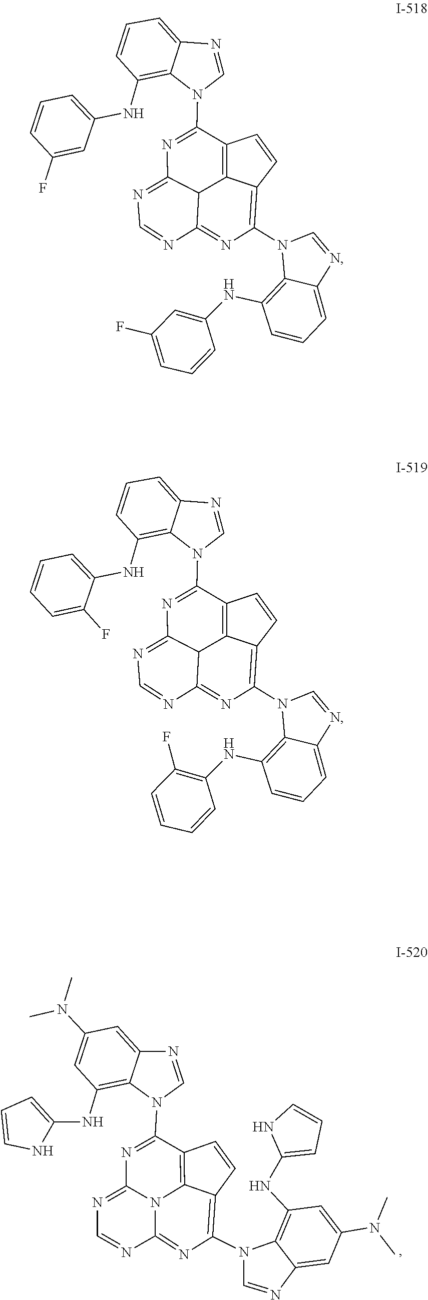 [SMILES: Fc1cccc(Nc2cccc3ncn(C4=NC5=NC=NC6=NC(n7cnc8cccc(Nc9cccc(F)c9)c87)=C7C=CC4=C7C56)c23)c1 ; Fc1ccccc1Nc1cccc2ncn(C3=NC4=NC=NC5=NC(n6cnc7cccc(Nc8ccccc8F)c76)=C6C=CC3=C6C45)c12 ; CN(C)c1cc(Nc2ccc[nH]2)c2c(c1)ncn2-c1nc2ncnc3nc(-n4cnc5cc(N(C)C)cc(Nc6ccc[nH]6)c54)c4ccc1c4n23]

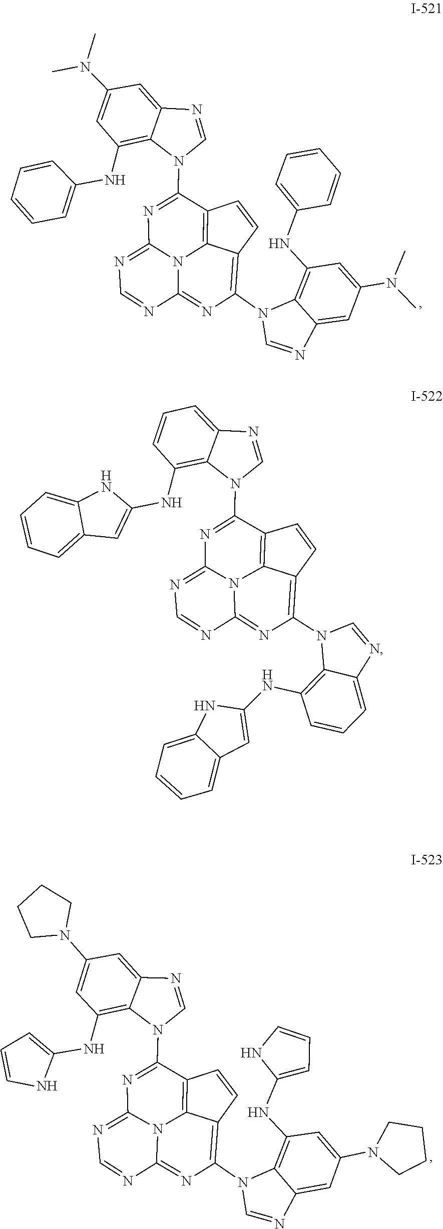

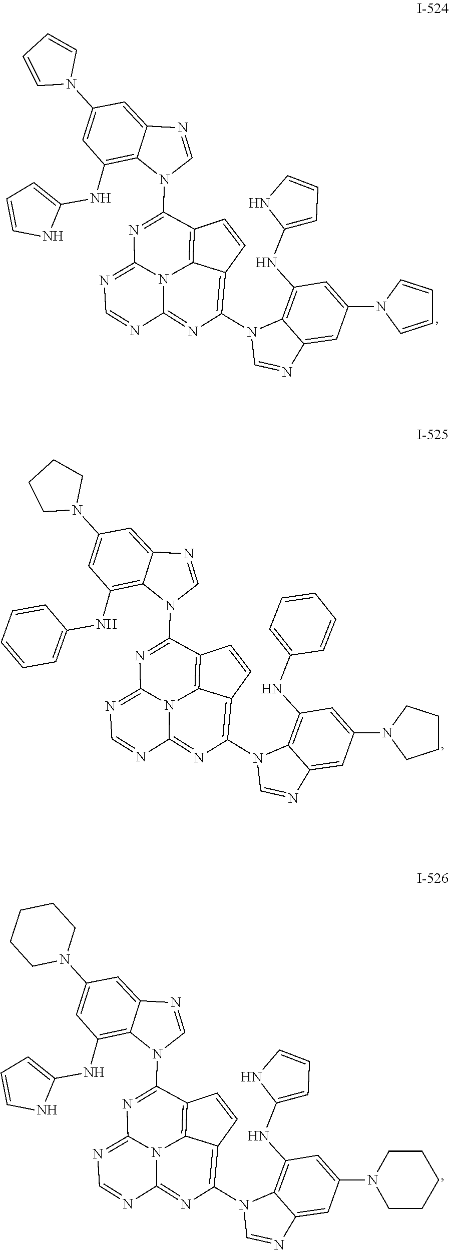 [SMILES: c1c[nH]c(Nc2cc(-n3cccc3)cc3ncn(-c4nc5ncnc6nc(-n7cnc8cc(-n9cccc9)cc(Nc9ccc[nH]9)c87)c7ccc4c7n56)c23)c1 ; c1ccc(Nc2cc(N3CCCC3)cc3ncn(-c4nc5ncnc6nc(-n7cnc8cc(N9CCCC9)cc(Nc9ccccc9)c87)c7ccc4c7n56)c23)cc1 ; c1c[nH]c(Nc2cc(N3CCCCC3)cc3ncn(-c4nc5ncnc6nc(-n7cnc8cc(N9CCCCC9)cc(Nc9ccc[nH]9)c87)c7ccc4c7n56)c23)c1]

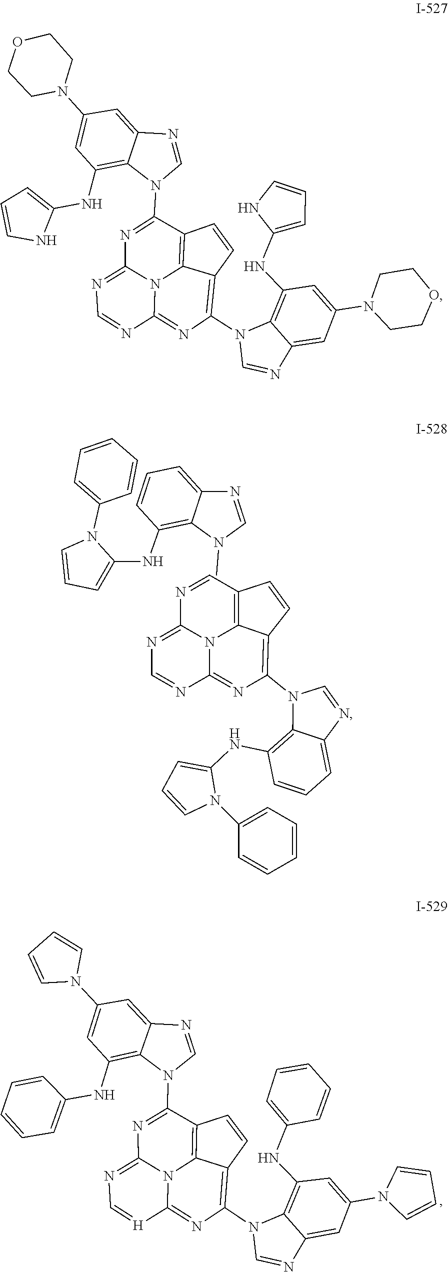

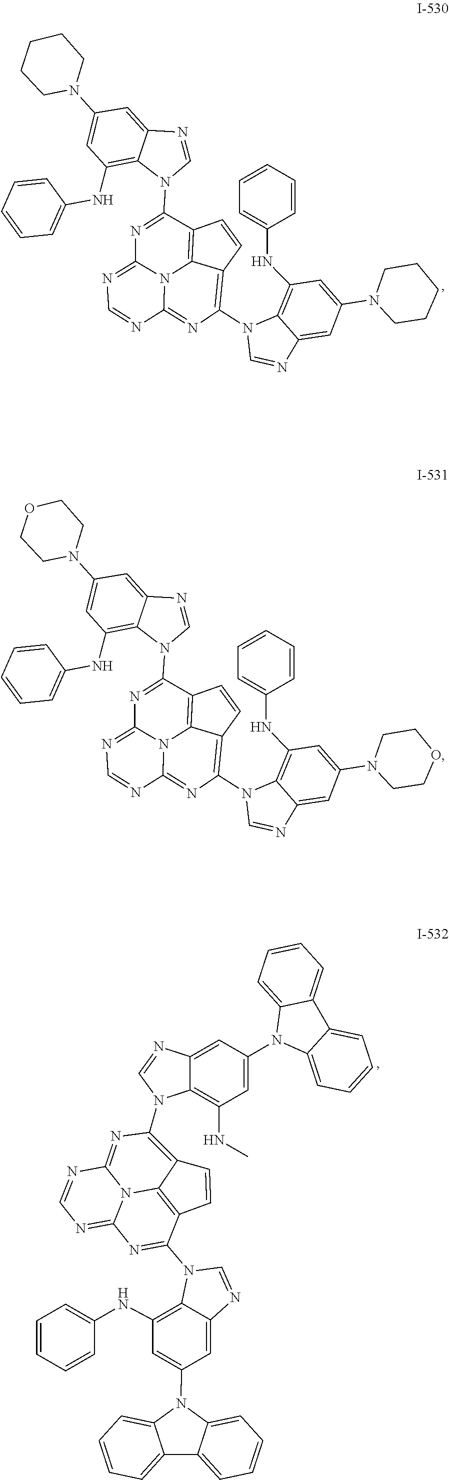 [SMILES: c1ccc(Nc2cc(N3CCCCC3)cc3ncn(-c4nc5ncnc6nc(-n7cnc8cc(N9CCCCC9)cc(Nc9ccccc9)c87)c7ccc4c7n56)c23)cc1 ; c1ccc(Nc2cc(N3CCOCC3)cc3ncn(-c4nc5ncnc6nc(-n7cnc8cc(N9CCOCC9)cc(Nc9ccccc9)c87)c7ccc4c7n56)c23)cc1 ; CNc1cc(-n2c3ccccc3c3ccccc32)cc2ncn(-c3nc4ncnc5nc(-n6cnc7cc(-n8c9ccccc9c9ccccc98)cc(Nc8ccccc8)c76)c6ccc3c6n45)c12]

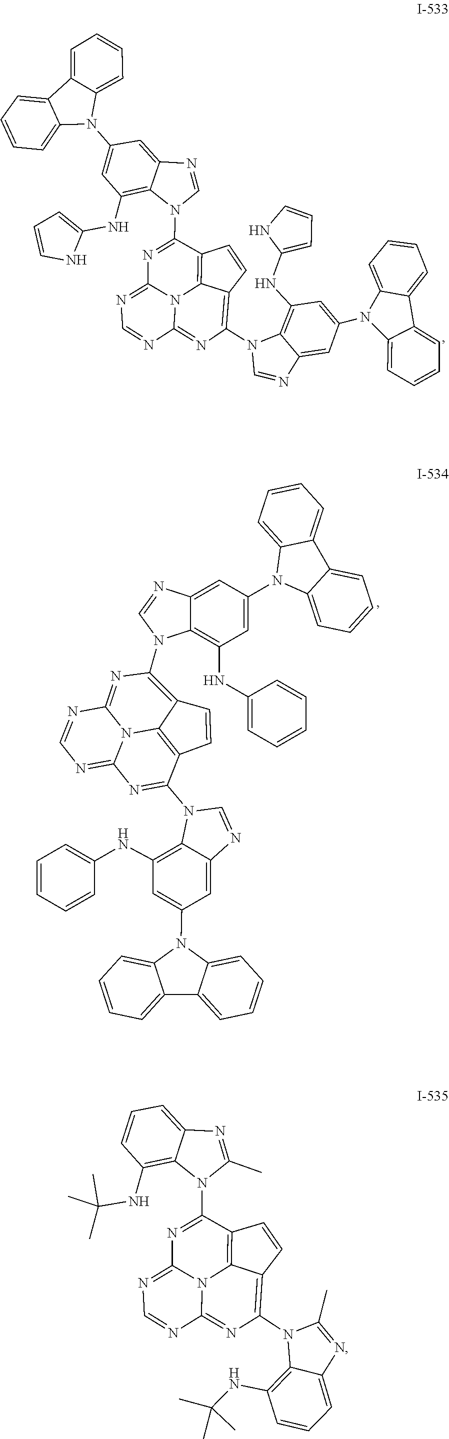 [SMILES: c1c[nH]c(Nc2cc(-n3c4ccccc4c4ccccc43)cc3ncn(-c4nc5ncnc6nc(-n7cnc8cc(-n9c%10ccccc%10c%10ccccc%109)cc(Nc9ccc[nH]9)c87)c7ccc4c7n56)c23)c1 ; c1ccc(Nc2cc(-n3c4ccccc4c4ccccc43)cc3ncn(-c4nc5ncnc6nc(-n7cnc8cc(-n9c%10ccccc%10c%10ccccc%109)cc(Nc9ccccc9)c87)c7ccc4c7n56)c23)cc1 ; Cc1nc2cccc(NC(C)(C)C)c2n1-c1nc2ncnc3nc(-n4c(C)nc5cccc(NC(C)(C)C)c54)c4ccc1c4n23]

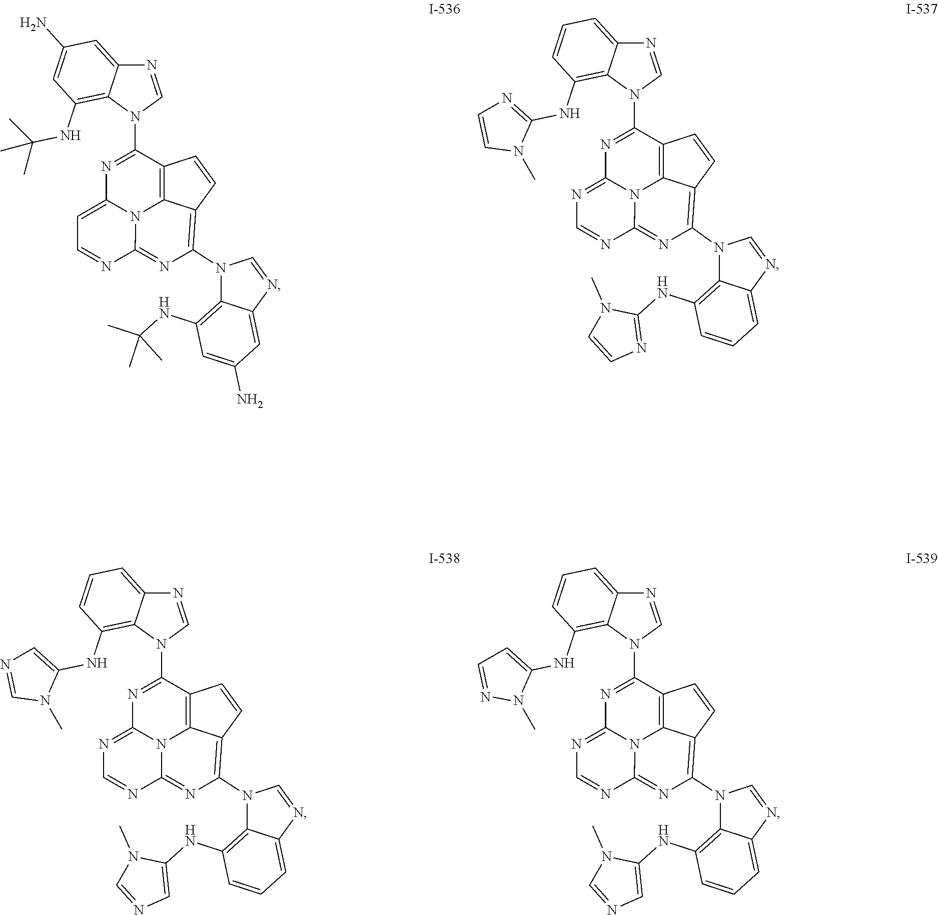 [SMILES: CC(C)(C)Nc1cc(N)cc2ncn(-c3nc4ccnc5nc(-n6cnc7cc(N)cc(NC(C)(C)C)c76)c6ccc3c6n45)c12 ; Cn1ccnc1Nc1cccc2ncn(-c3nc4ncnc5nc(-n6cnc7cccc(Nc8nccn8C)c76)c6ccc3c6n45)c12 ; Cn1cncc1Nc1cccc2ncn(-c3nc4ncnc5nc(-n6cnc7cccc(Nc8cncn8C)c76)c6ccc3c6n45)c12 ; Cn1cncc1Nc1cccc2ncn(-c3nc4ncnc5nc(-n6cnc7cccc(Nc8ccnn8C)c76)c6ccc3c6n45)c12]

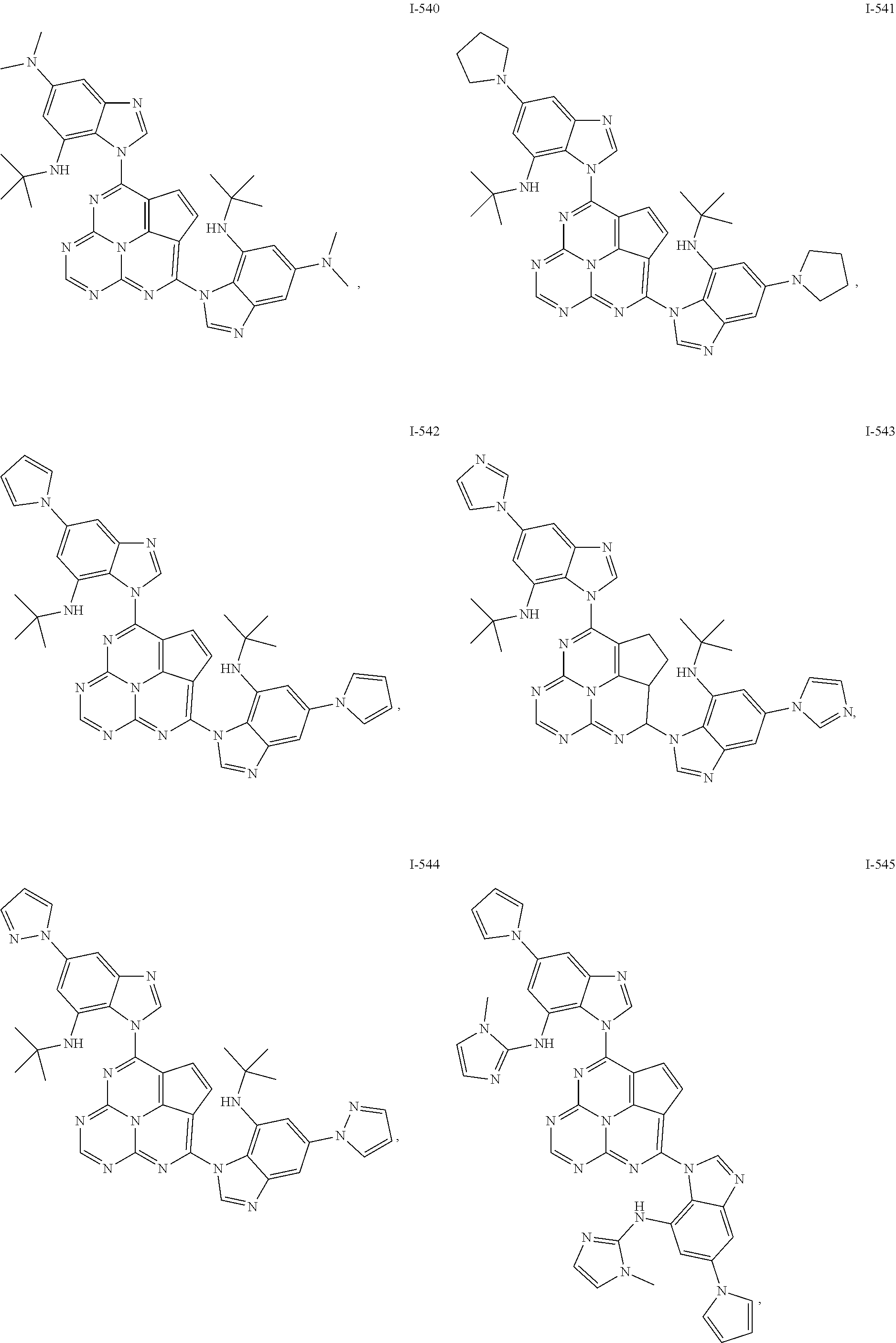

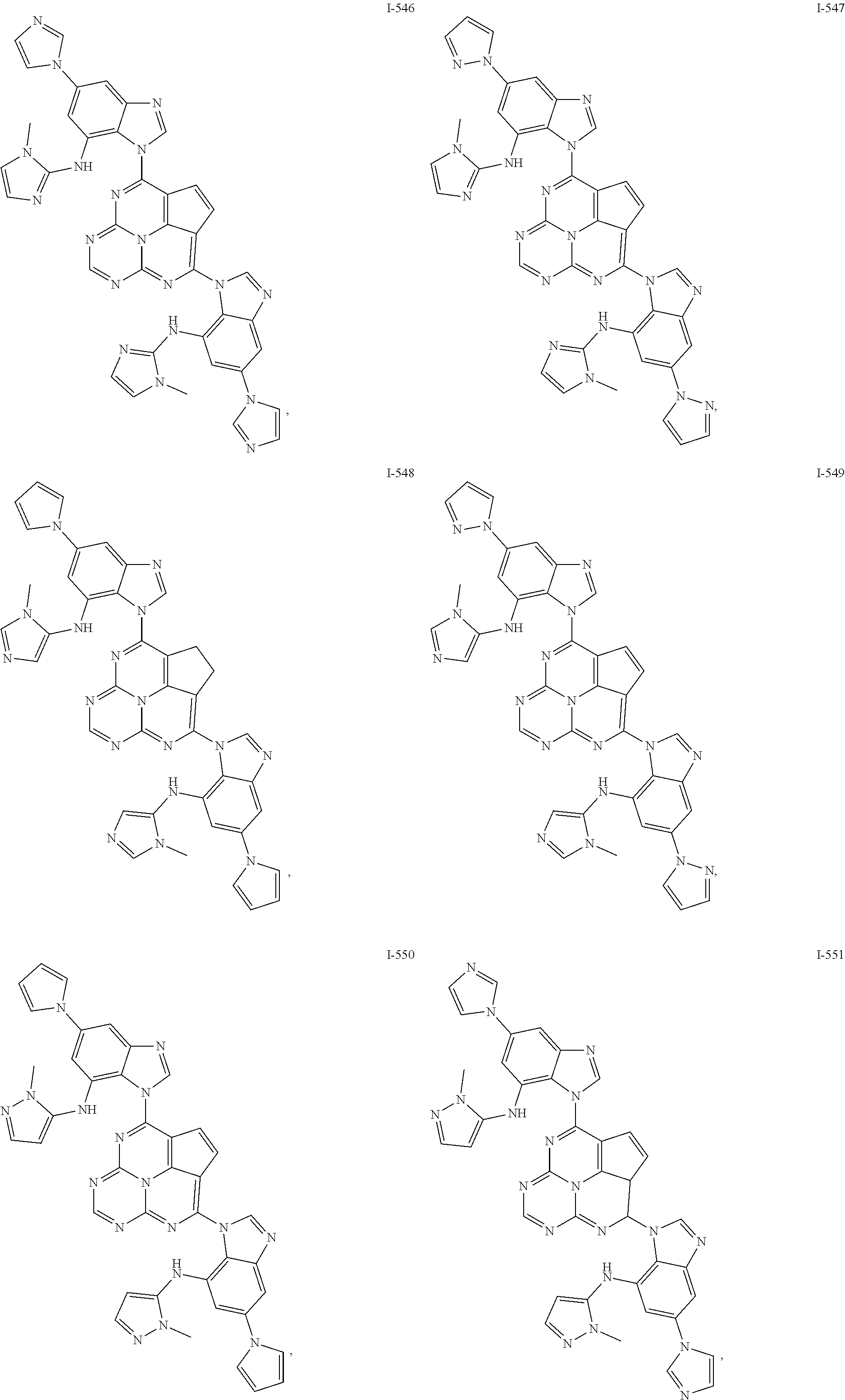

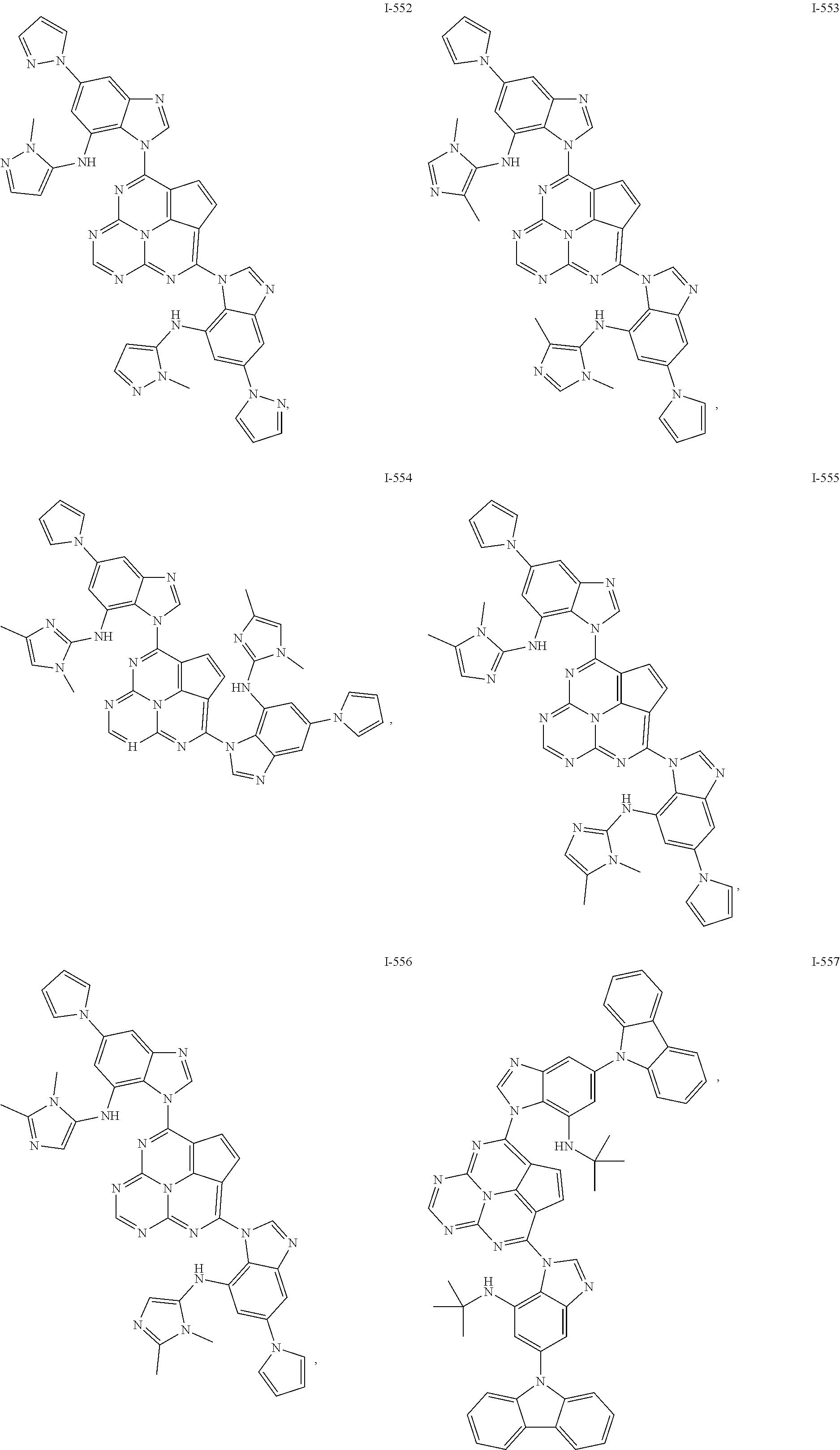

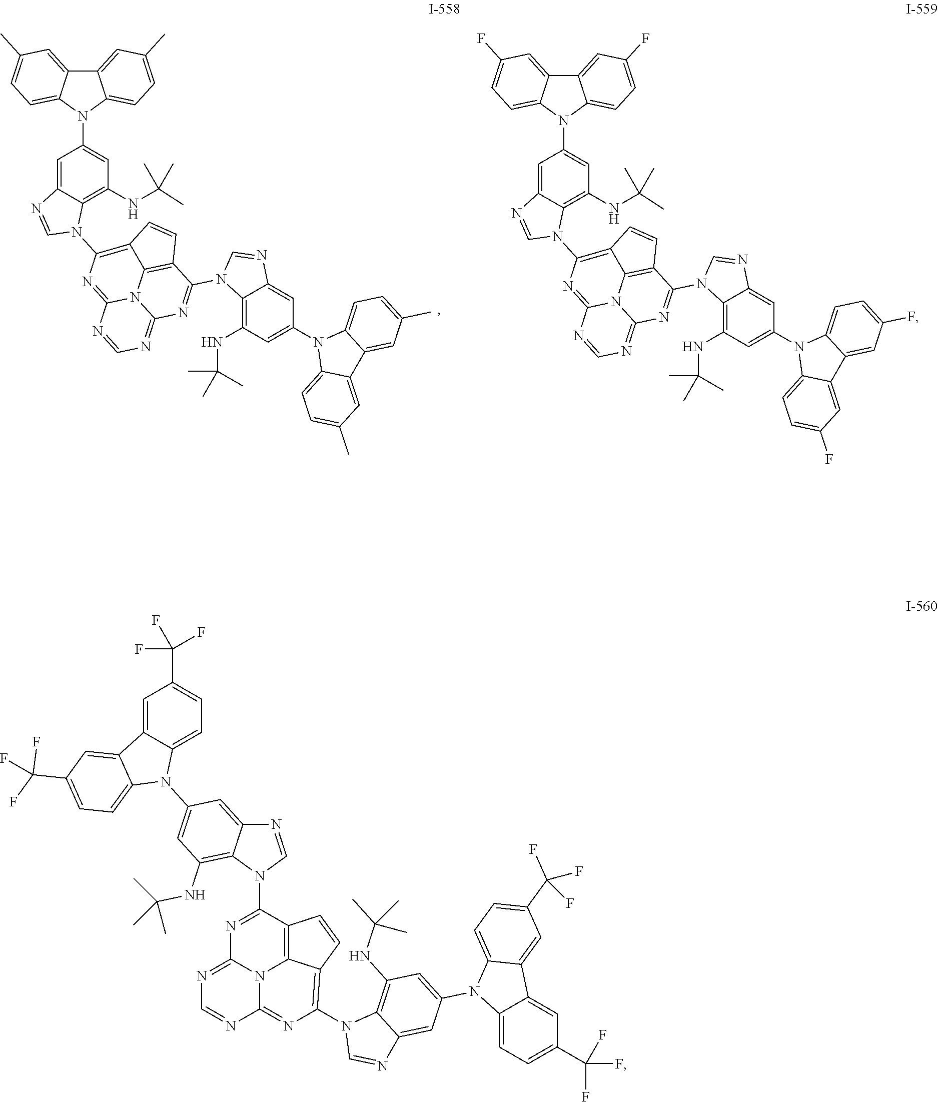

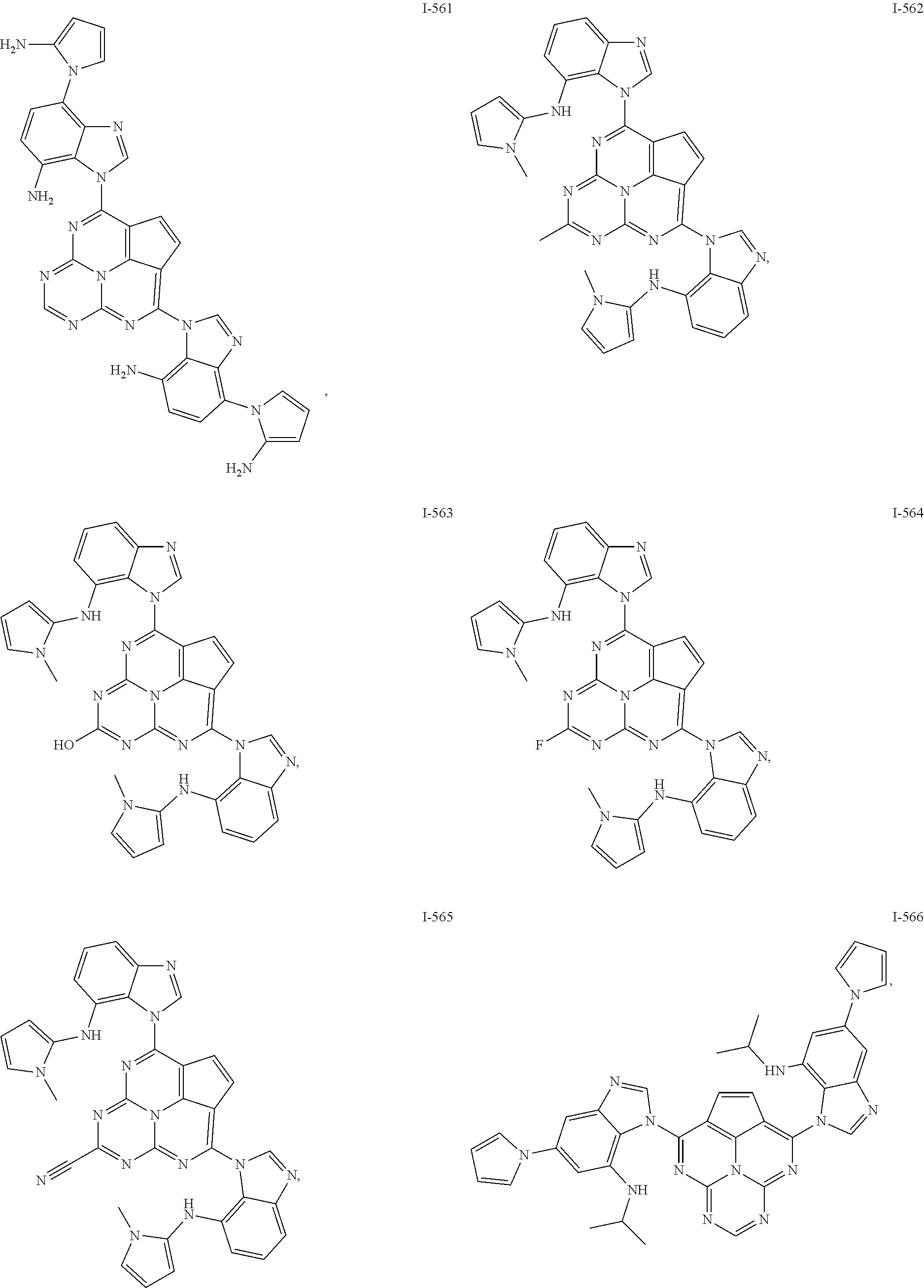

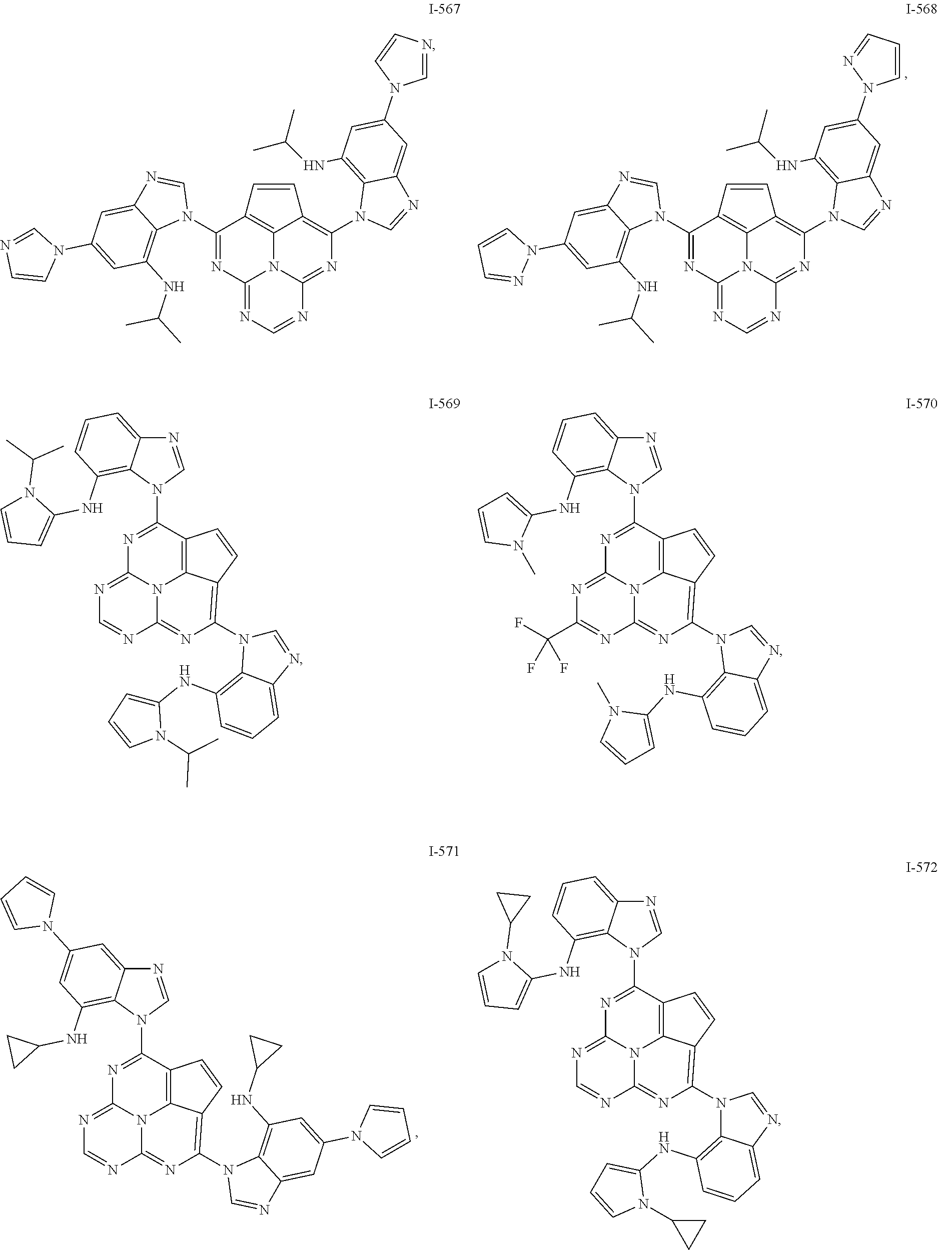

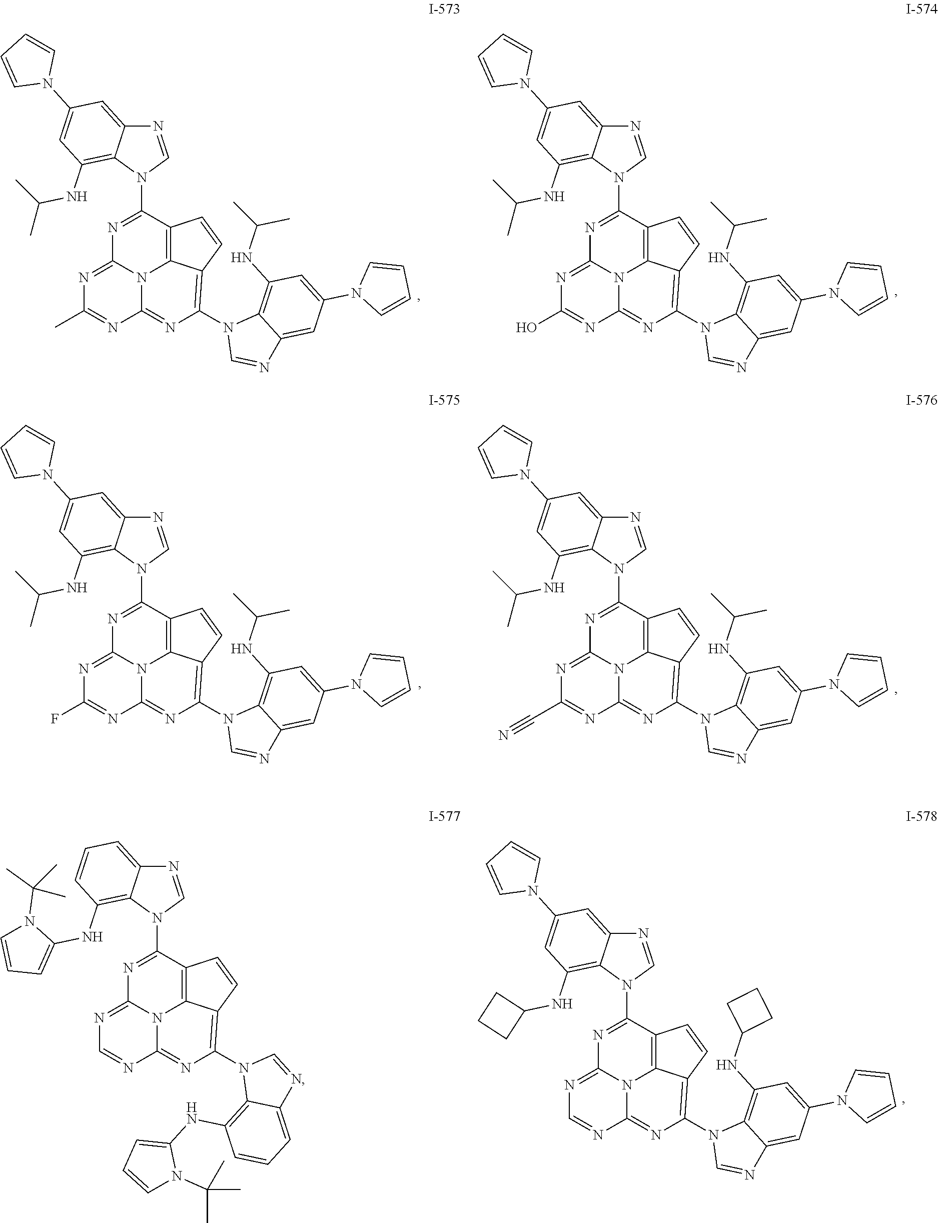

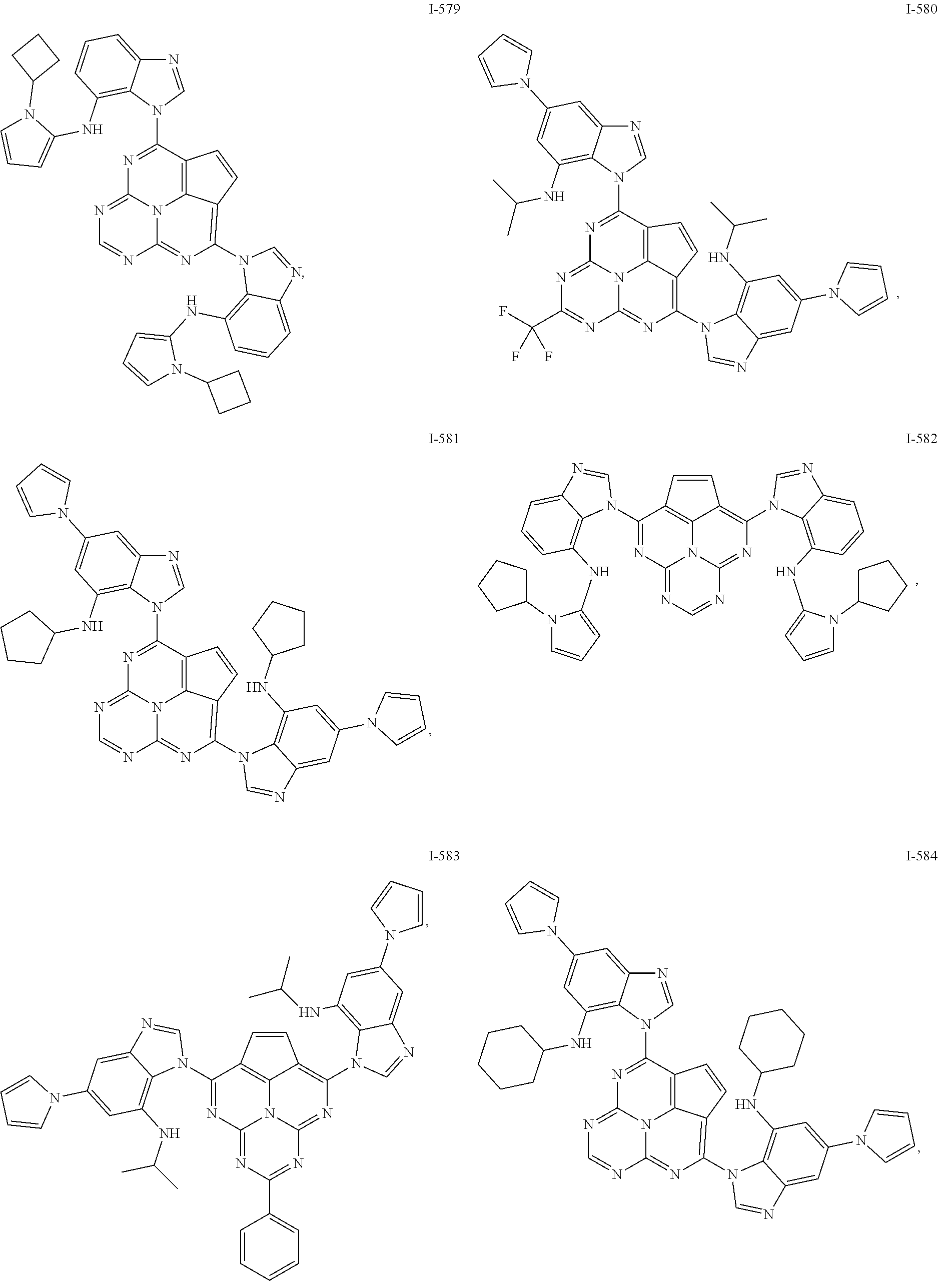

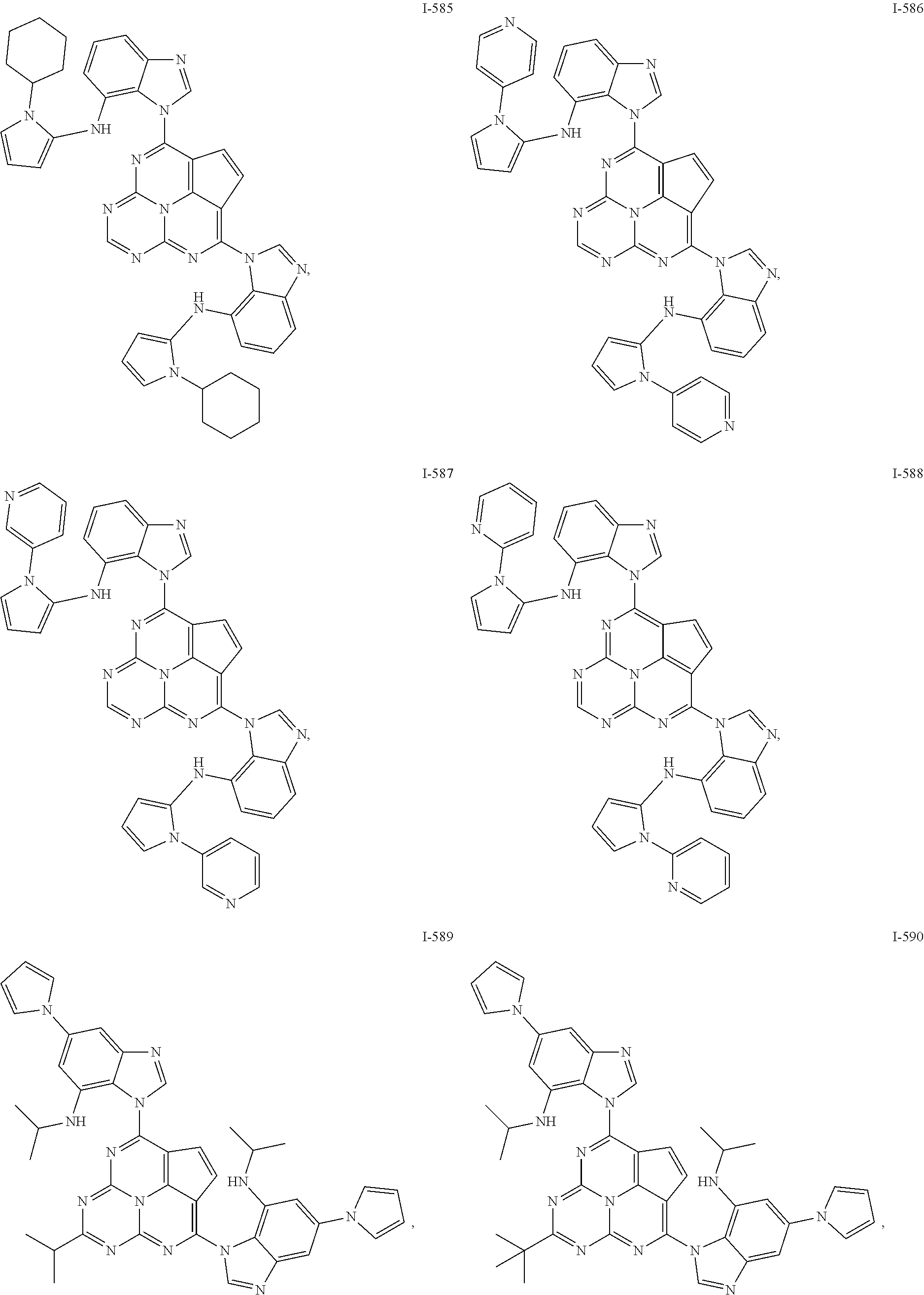

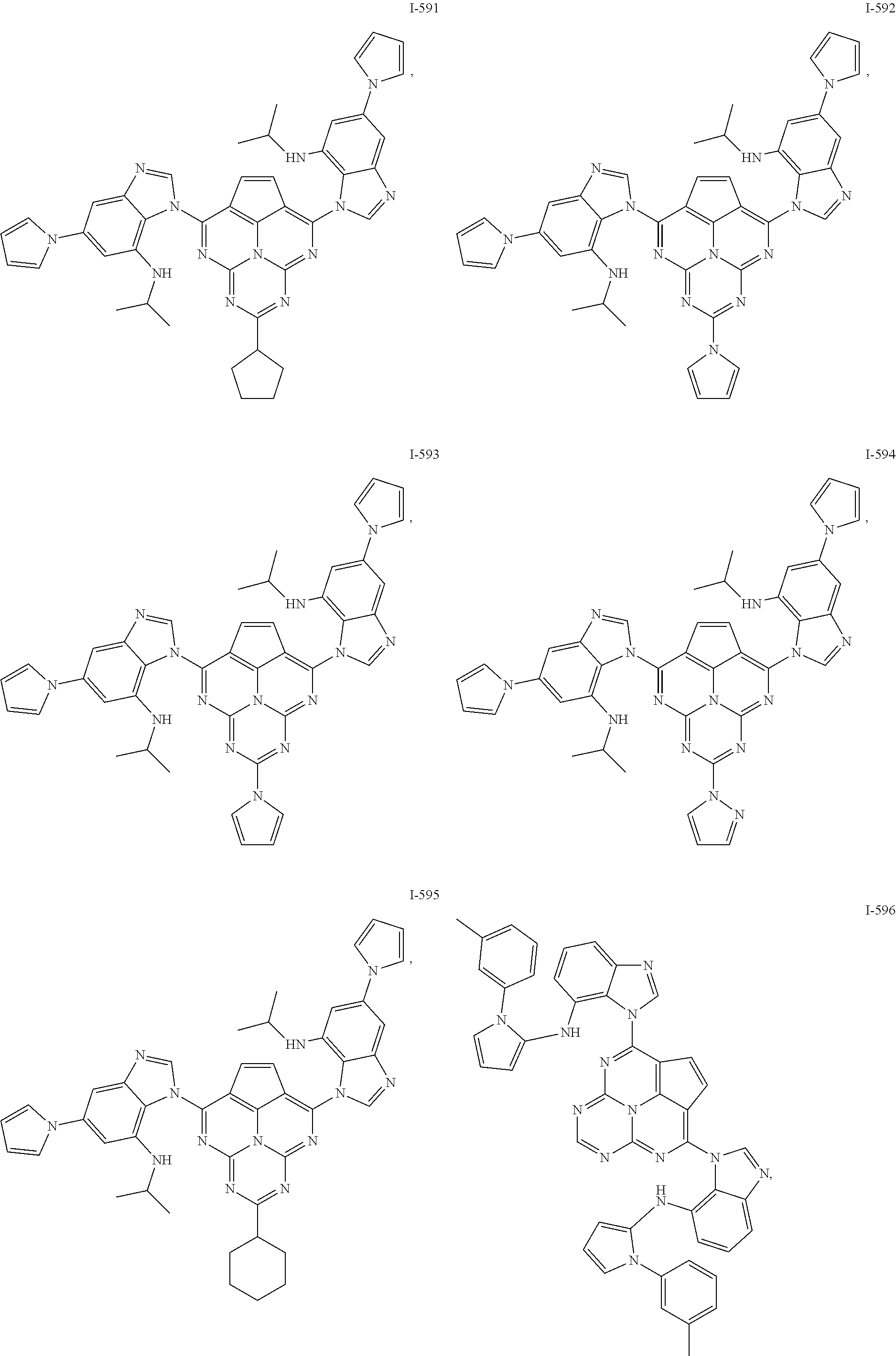

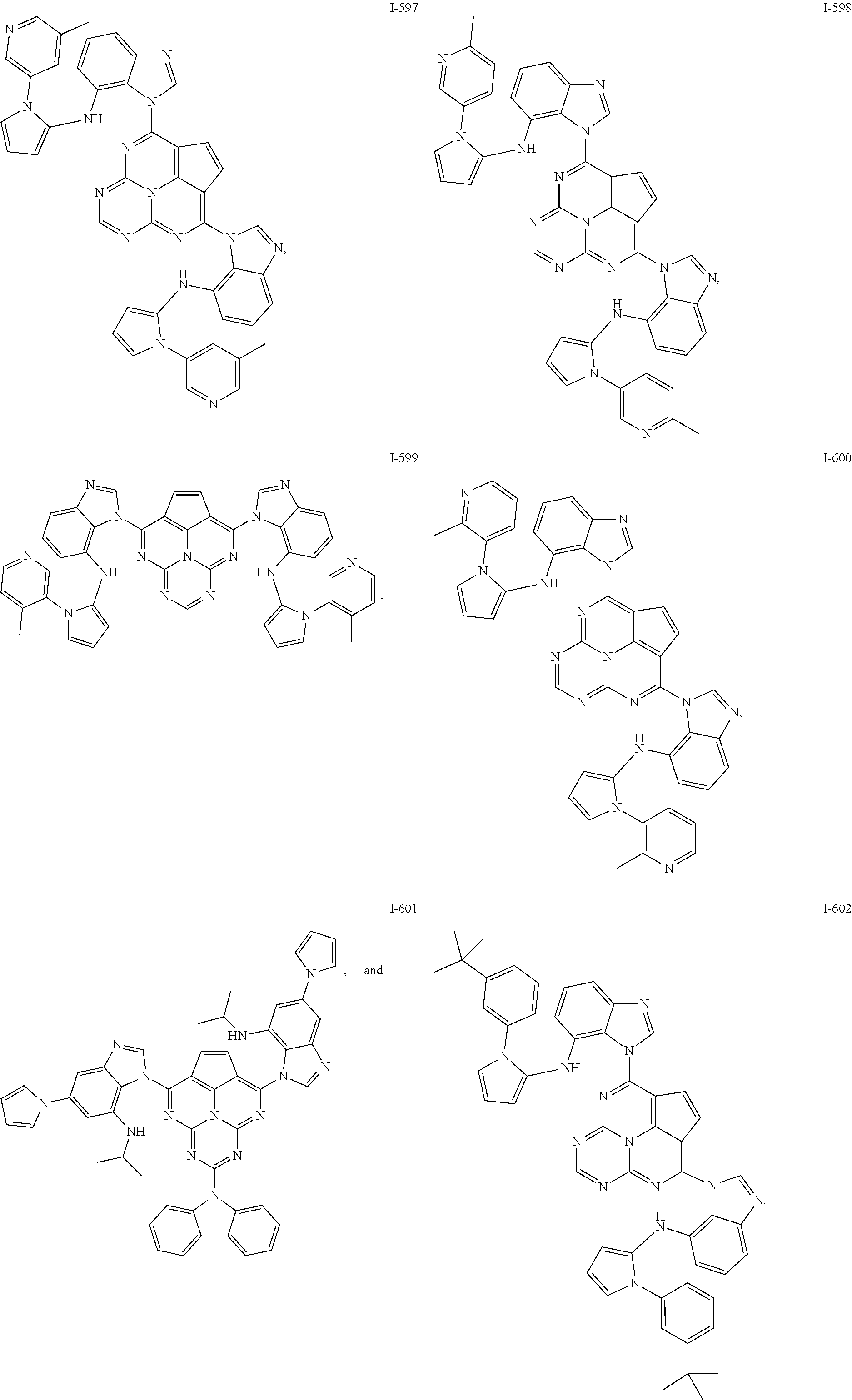

In some embodiments, the compound has a structure of Formula I-b

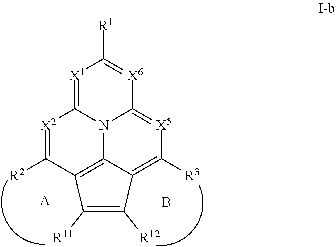

wherein ring A and ring B are each independently a 5-membered or 6-membered carbocycle or heterocycle, optionally an aromatic or heteroaromatic cycle, unsubstituted or substituted with one or more substituents independently selected from R¹⁰.

In some embodiments, the heterocycle is a nitrogen-containing heterocycle.

In some embodiments, R¹¹ and R¹² are nitrogen.

In some embodiment, the compound is selected from

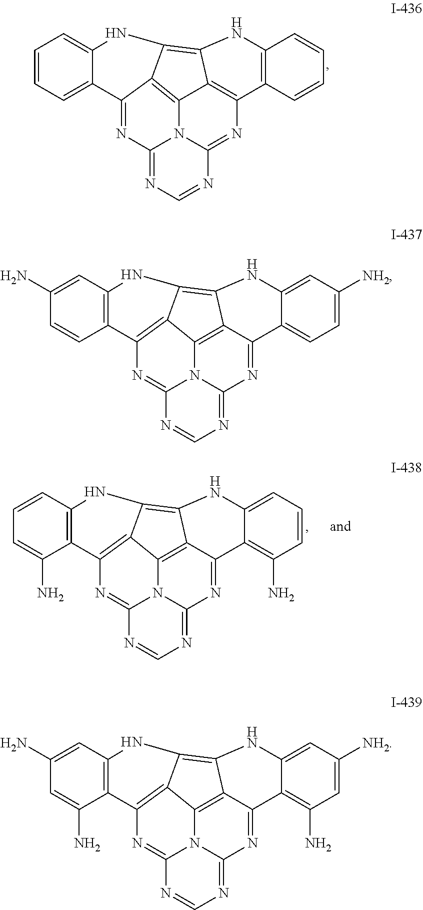 [SMILES: c1ccc2c(c1)[nH]c1c3[nH]c4ccccc4c4nc5ncnc6nc2c1c(c43)n65 ; Nc1ccc2c(c1)[nH]c1c3[nH]c4cc(N)ccc4c4nc5ncnc6nc2c1c(c43)n65 ; Nc1cccc2[nH]c3c4[nH]c5cccc(N)c5c5nc6ncnc7nc(c12)c3c(c54)n76 ; Nc1cc(N)c2c(c1)[nH]c1c3[nH]c4cc(N)cc(N)c4c4nc5ncnc6nc2c1c(c43)n65]

In some embodiments, the compound has a structure of Formula I-c

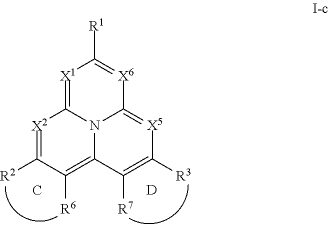

wherein ring C and ring D are each independently a 5-membered or 6-membered carbocycle or heterocycle, optionally an aromatic or heteroaromatic cycle, unsubstituted or substituted with one or more substituents independently selected from R¹⁰.

In some embodiments, ring C and ring D are each independently selected from nitrogen-containing heterocycles and sulfur-containing heterocycles.

In some embodiments, the compound is

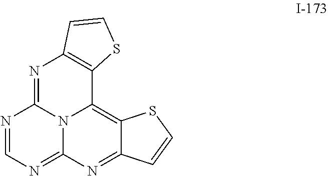 [SMILES: C1=NC2=Nc3ccsc3C3=c4sccc4=NC(=N1)N23]

In some embodiments, the compound has a structure of Formula I-d

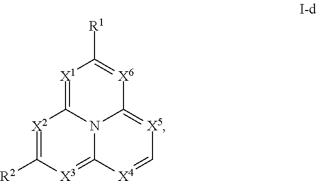

and wherein R¹ and R² are each independently selected from aryl and heteroaryl, each unsubstituted or substituted with one or more substituents independently selected from R¹⁰.

In some embodiments, R¹ and R² are each independently selected from phenyl, pyrrole, furan, thiophene, indole, benzofuran, benzothiophene, benzoimidazole, indazole, indoline, quinolinone, and pyridine.

In some embodiments, the compound is selected from

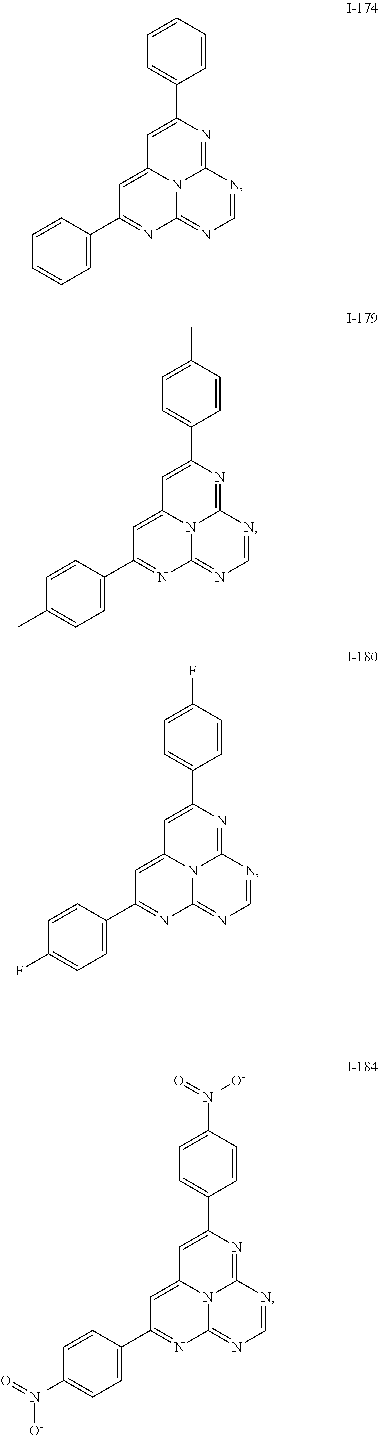 [SMILES: C1=NC2=NC(c3ccccc3)=CC3=CC(c4ccccc4)=NC(=N1)N32 ; Cc1ccc(C2=CC3=CC(c4ccc(C)cc4)=NC4=NC=NC(=N2)N34)cc1 ; Fc1ccc(C2=CC3=CC(c4ccc(F)cc4)=NC4=NC=NC(=N2)N34)cc1 ; O=[N+]([O-])c1ccc(C2=CC3=CC(c4ccc([N+](=O)[O-])cc4)=NC4=NC=NC(=N2)N34)cc1]

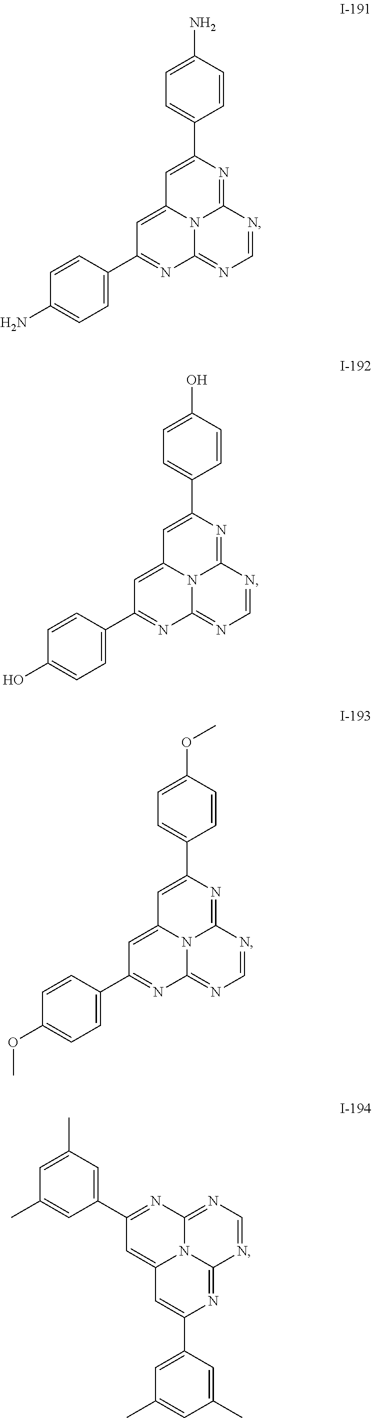

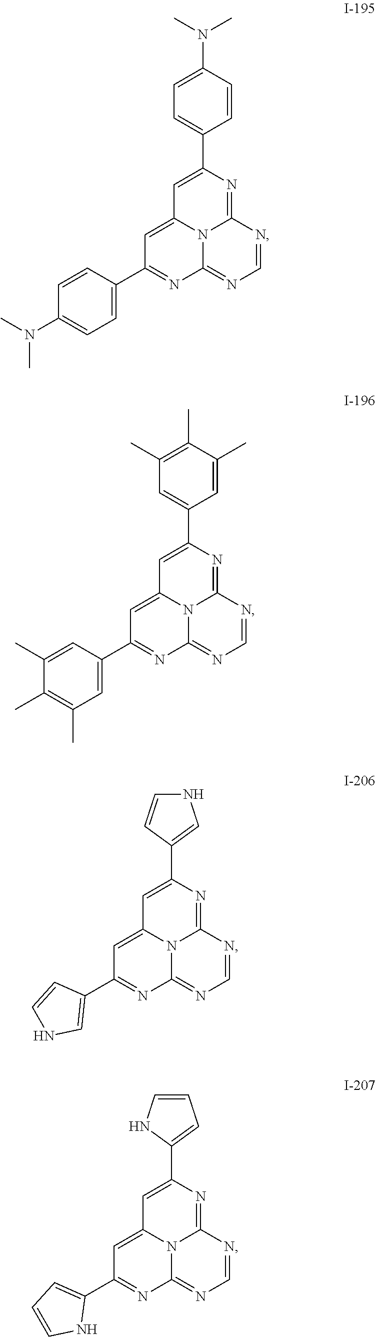

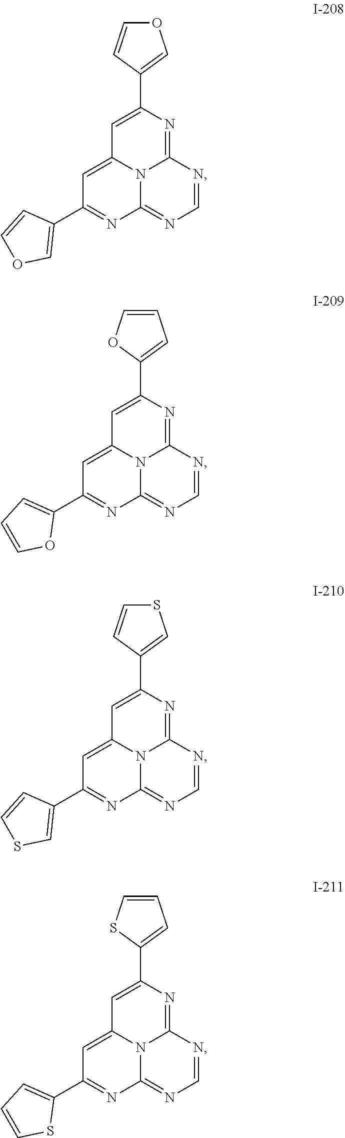 [SMILES: C1=NC2=NC(c3ccoc3)=CC3=CC(c4ccoc4)=NC(=N1)N32 ; C1=NC2=NC(c3ccco3)=CC3=CC(c4ccco4)=NC(=N1)N32 ; C1=NC2=NC(c3ccsc3)=CC3=CC(c4ccsc4)=NC(=N1)N32 ; C1=NC2=NC(c3cccs3)=CC3=CC(c4cccs4)=NC(=N1)N32]

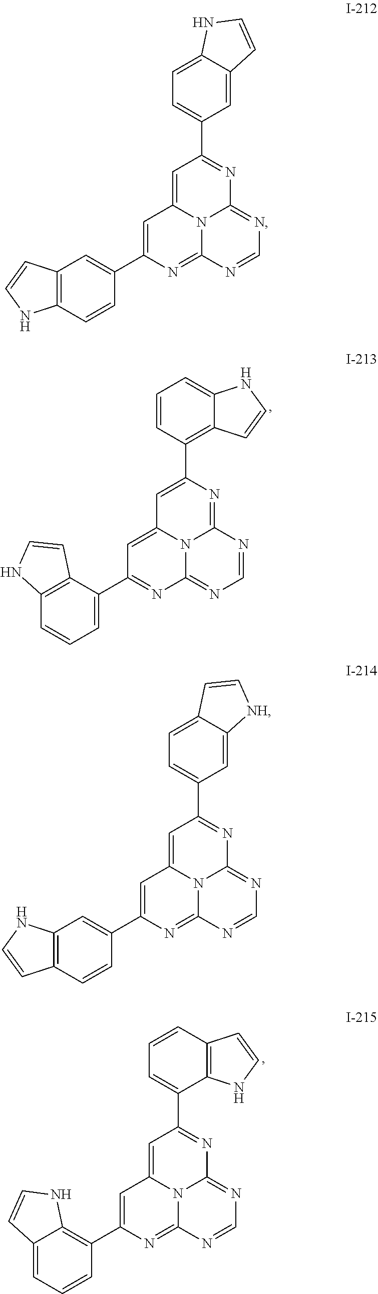 [SMILES: C1=NC2=NC(c3ccc4[nH]ccc4c3)=CC3=CC(c4ccc5[nH]ccc5c4)=NC(=N1)N32 ; C1=NC2=NC(c3cccc4[nH]ccc34)=CC3=CC(c4cccc5[nH]ccc45)=NC(=N1)N32 ; C1=NC2=NC(c3ccc4cc[nH]c4c3)=CC3=CC(c4ccc5cc[nH]c5c4)=NC(=N1)N32 ; C1=NC2=NC(c3cccc4cc[nH]c34)=CC3=CC(c4cccc5cc[nH]c45)=NC(=N1)N32]

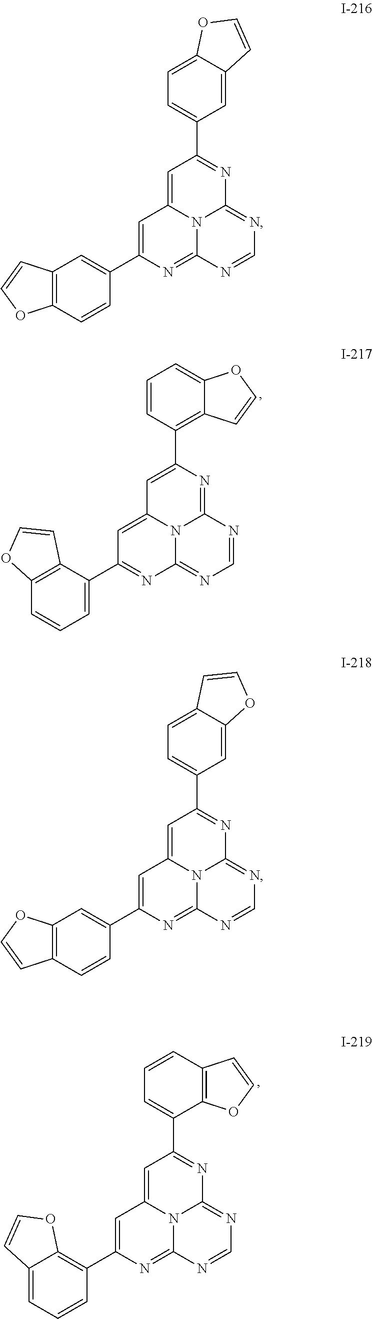 [SMILES: C1=NC2=NC(c3ccc4occc4c3)=CC3=CC(c4ccc5occc5c4)=NC(=N1)N32 ; C1=NC2=NC(c3cccc4occc34)=CC3=CC(c4cccc5occc45)=NC(=N1)N32 ; C1=NC2=NC(c3ccc4ccoc4c3)=CC3=CC(c4ccc5ccoc5c4)=NC(=N1)N32 ; C1=NC2=NC(c3cccc4ccoc34)=CC3=CC(c4cccc5ccoc45)=NC(=N1)N32]

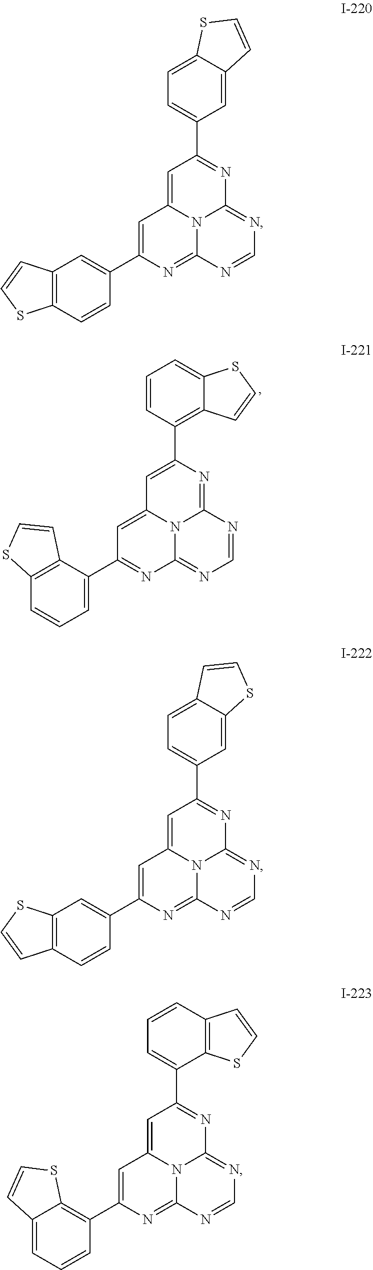 [SMILES: C1=NC2=NC(c3ccc4sccc4c3)=CC3=CC(c4ccc5sccc5c4)=NC(=N1)N32 ; C1=NC2=NC(c3cccc4sccc34)=CC3=CC(c4cccc5sccc45)=NC(=N1)N32 ; C1=NC2=NC(c3ccc4ccsc4c3)=CC3=CC(c4ccc5ccsc5c4)=NC(=N1)N32 ; C1=NC2=NC(c3cccc4ccsc34)=CC3=CC(c4cccc5ccsc45)=NC(=N1)N32]

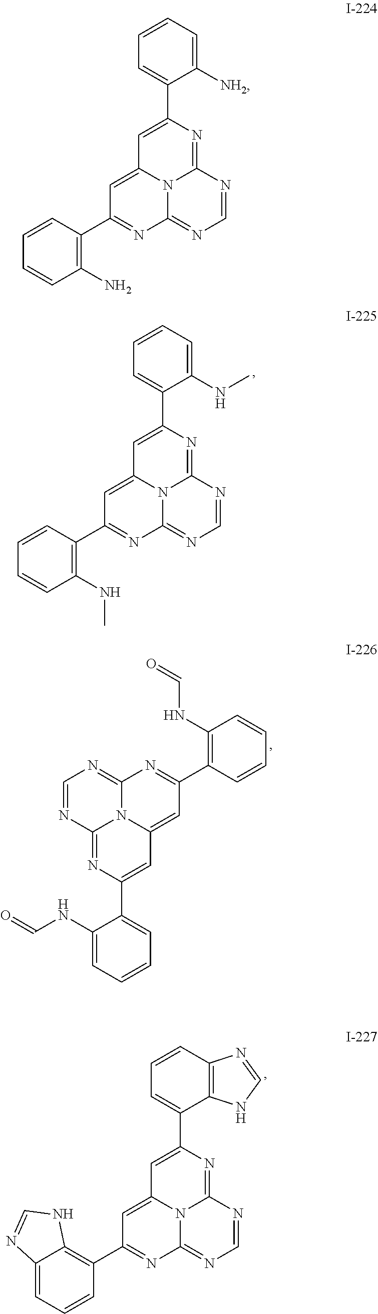 [SMILES: Nc1ccccc1C1=CC2=CC(c3ccccc3N)=NC3=NC=NC(=N1)N23 ; CNc1ccccc1C1=CC2=CC(c3ccccc3NC)=NC3=NC=NC(=N1)N23 ; O=CNc1ccccc1C1=CC2=CC(c3ccccc3NC=O)=NC3=NC=NC(=N1)N23 ; C1=NC2=NC(c3cccc4nc[nH]c34)=CC3=CC(c4cccc5nc[nH]c45)=NC(=N1)N32]

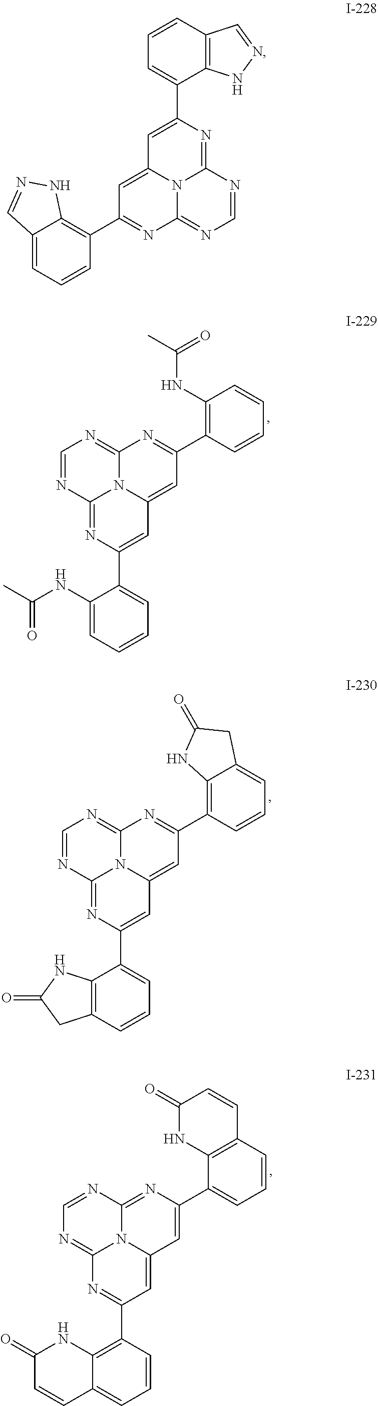 [SMILES: C1=NC2=NC(c3cccc4cn[nH]c34)=CC3=CC(c4cccc5cn[nH]c45)=NC(=N1)N32 ; CC(=O)Nc1ccccc1C1=CC2=CC(c3ccccc3NC(C)=O)=NC3=NC=NC(=N1)N23 ; O=C1Cc2cccc(C3=CC4=CC(c5cccc6c5NC(=O)C6)=NC5=NC=NC(=N3)N45)c2N1 ; O=c1ccc2cccc(C3=CC4=CC(c5cccc6ccc(=O)[nH]c56)=NC5=NC=NC(=N3)N45)c2[nH]1]

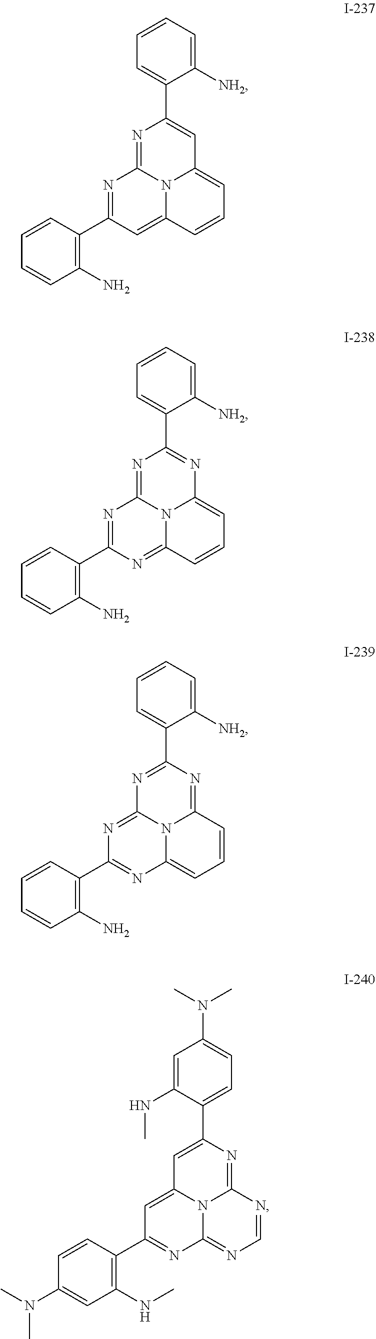 [SMILES: Nc1ccccc1C1=CC2=CC=CC3=CC(c4ccccc4N)=NC(=N1)N23 ; Nc1ccccc1C1=NC2=CC=CC3=NC(c4ccccc4N)=NC(=N1)N23 ; Nc1ccccc1C1=NC2=CC=CC3=NC(c4ccccc4N)=NC(=N1)N23 ; CNc1cc(N(C)C)ccc1C1=CC2=CC(c3ccc(N(C)C)cc3NC)=NC3=NC=NC(=N1)N23]

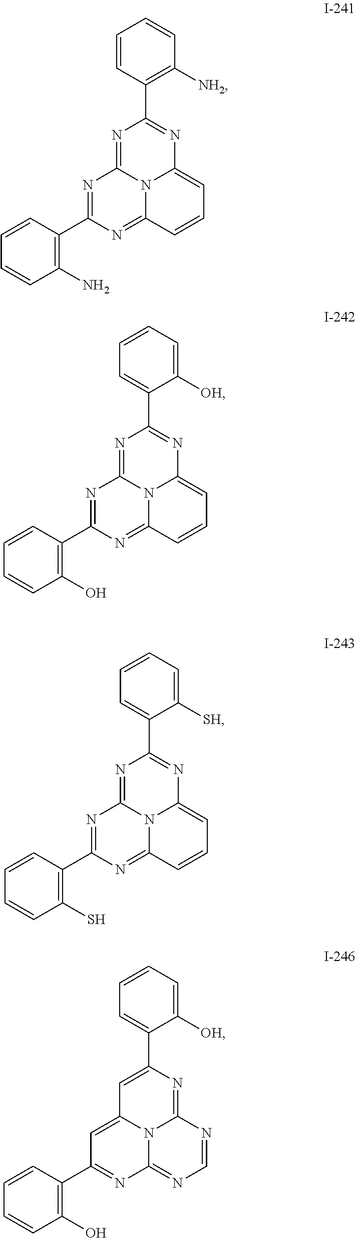 [SMILES: Nc1ccccc1C1=NC2=CC=CC3=NC(c4ccccc4N)=NC(=N1)N23 ; Oc1ccccc1C1=NC2=CC=CC3=NC(c4ccccc4O)=NC(=N1)N23 ; Sc1ccccc1C1=NC2=CC=CC3=NC(c4ccccc4S)=NC(=N1)N23 ; Oc1ccccc1C1=CC2=CC(c3ccccc3O)=NC3=NC=NC(=N1)N23]

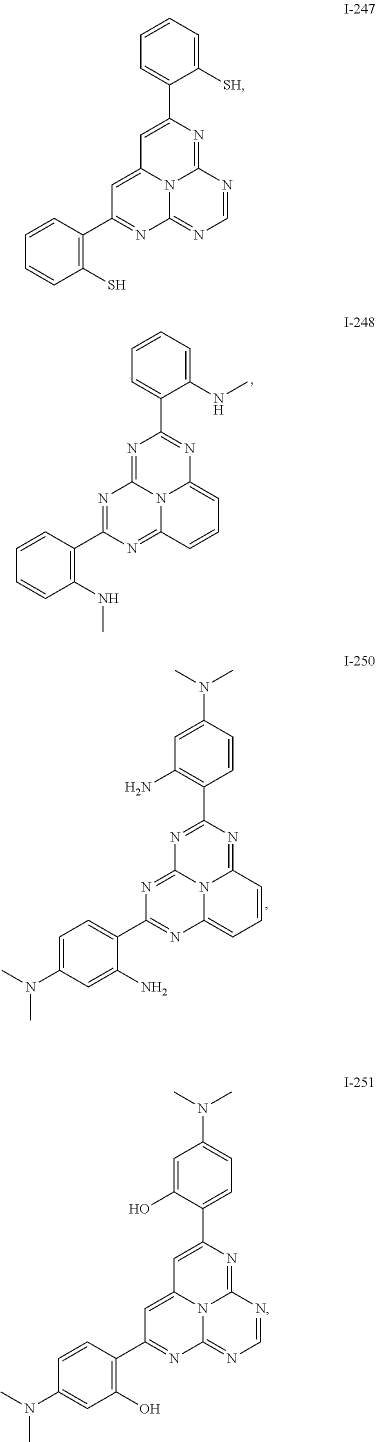

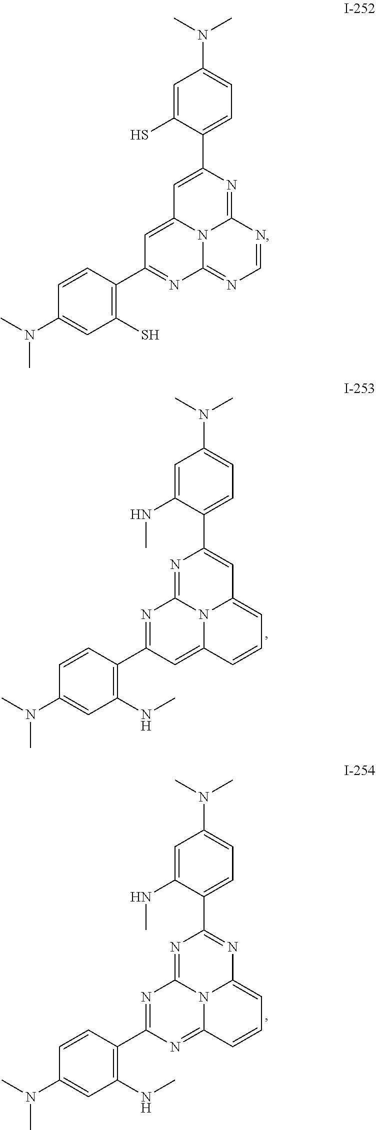 [SMILES: CN(C)c1ccc(C2=CC3=CC(c4ccc(N(C)C)cc4S)=NC4=NC=NC(=N2)N34)c(S)c1 ; CNc1cc(N(C)C)ccc1C1=CC2=CC=CC3=CC(c4ccc(N(C)C)cc4NC)=NC(=N1)N23 ; CNc1cc(N(C)C)ccc1C1=NC2=CC=CC3=NC(c4ccc(N(C)C)cc4NC)=NC(=N1)N23]

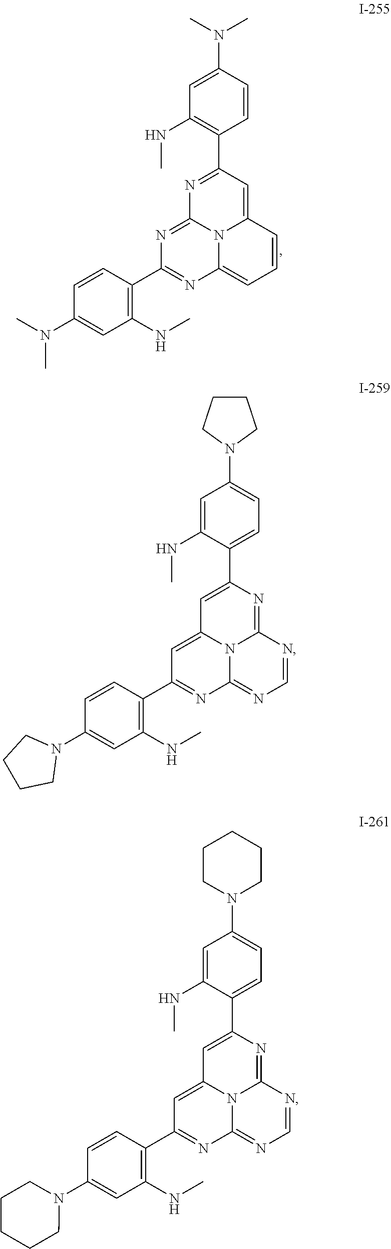 [SMILES: CNc1cc(N(C)C)ccc1C1=NC2=NC(c3ccc(N(C)C)cc3NC)=NC3=CC=CC(=C1)N32 ; CNc1cc(N2CCCC2)ccc1C1=CC2=CC(c3ccc(N4CCCC4)cc3NC)=NC3=NC=NC(=N1)N23 ; CNc1cc(N2CCCCC2)ccc1C1=CC2=CC(c3ccc(N4CCCCC4)cc3NC)=NC3=NC=NC(=N1)N23]

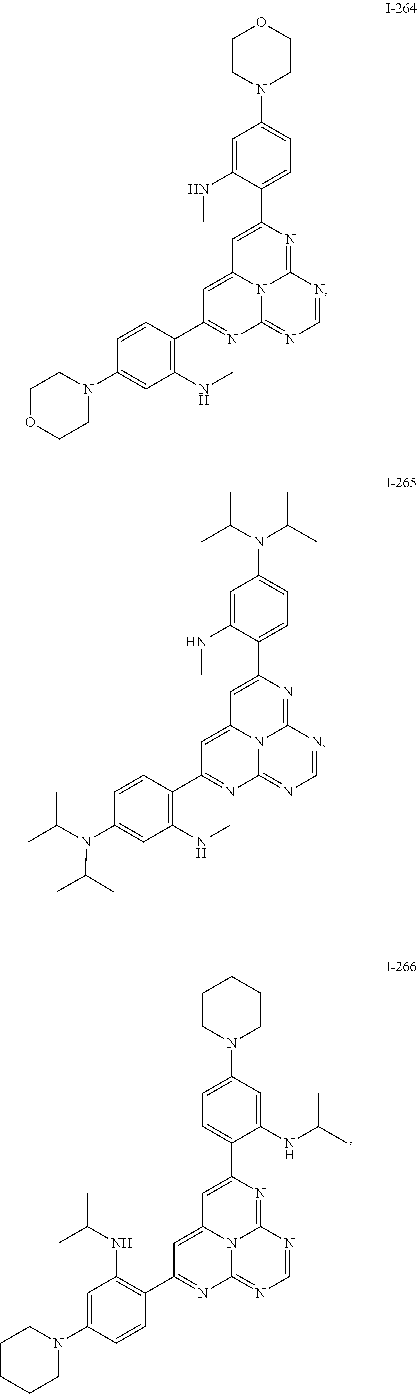 [SMILES: CNc1cc(N2CCOCC2)ccc1C1=CC2=CC(c3ccc(N4CCOCC4)cc3NC)=NC3=NC=NC(=N1)N23 ; CNc1cc(N(C(C)C)C(C)C)ccc1C1=CC2=CC(c3ccc(N(C(C)C)C(C)C)cc3NC)=NC3=NC=NC(=N1)N23 ; CC(C)Nc1cc(N2CCCCC2)ccc1C1=CC2=CC(c3ccc(N4CCCCC4)cc3NC(C)C)=NC3=NC=NC(=N1)N23]

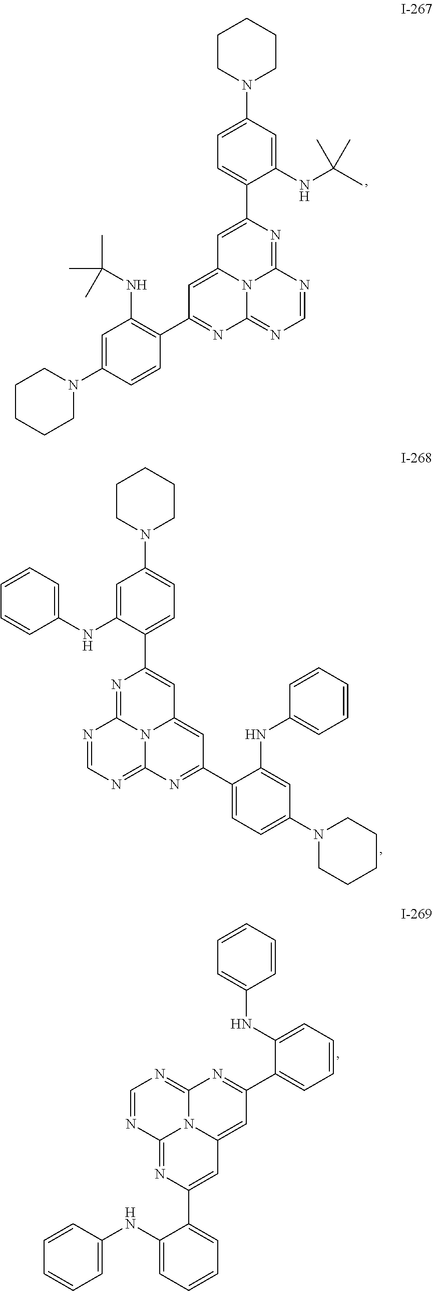 [SMILES: CC(C)(C)Nc1cc(N2CCCCC2)ccc1C1=CC2=CC(c3ccc(N4CCCCC4)cc3NC(C)(C)C)=NC3=NC=NC(=N1)N23 ; C1=NC2=NC(c3ccc(N4CCCCC4)cc3Nc3ccccc3)=CC3=CC(c4ccc(N5CCCCC5)cc4Nc4ccccc4)=NC(=N1)N32 ; C1=NC2=NC(c3ccccc3Nc3ccccc3)=CC3=CC(c4ccccc4Nc4ccccc4)=NC(=N1)N32]

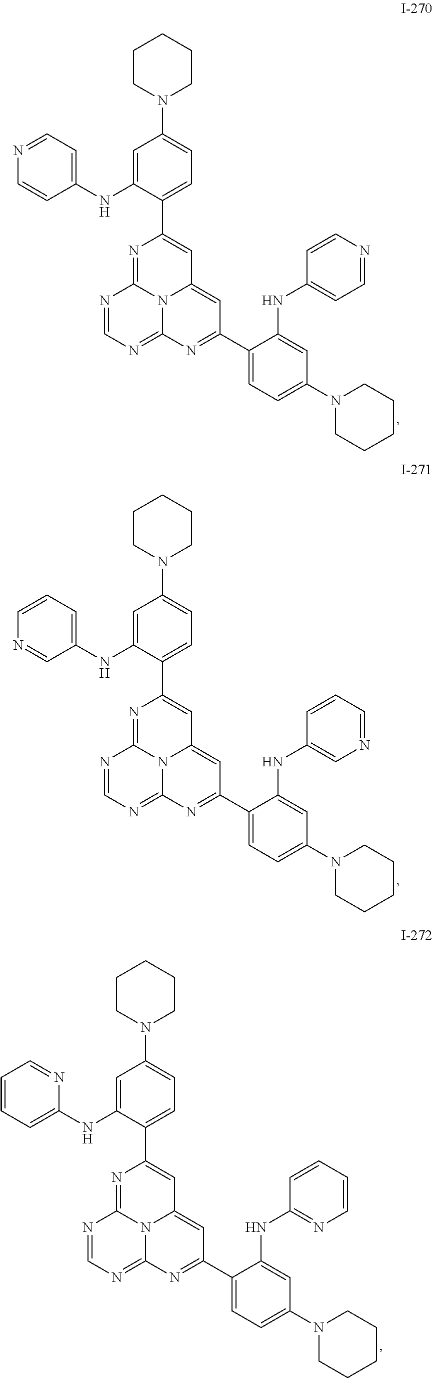 [SMILES: C1=NC2=NC(c3ccc(N4CCCCC4)cc3Nc3ccncc3)=CC3=CC(c4ccc(N5CCCCC5)cc4Nc4ccncc4)=NC(=N1)N32 ; C1=NC2=NC(c3ccc(N4CCCCC4)cc3Nc3cccnc3)=CC3=CC(c4ccc(N5CCCCC5)cc4Nc4cccnc4)=NC(=N1)N32 ; C1=NC2=NC(c3ccc(N4CCCCC4)cc3Nc3ccccn3)=CC3=CC(c4ccc(N5CCCCC5)cc4Nc4ccccn4)=NC(=N1)N32]

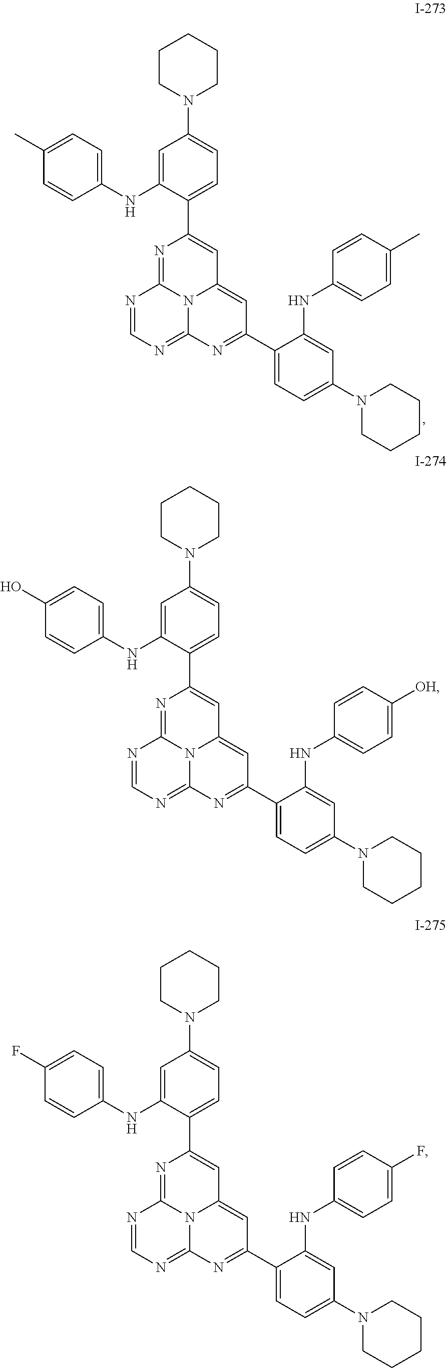

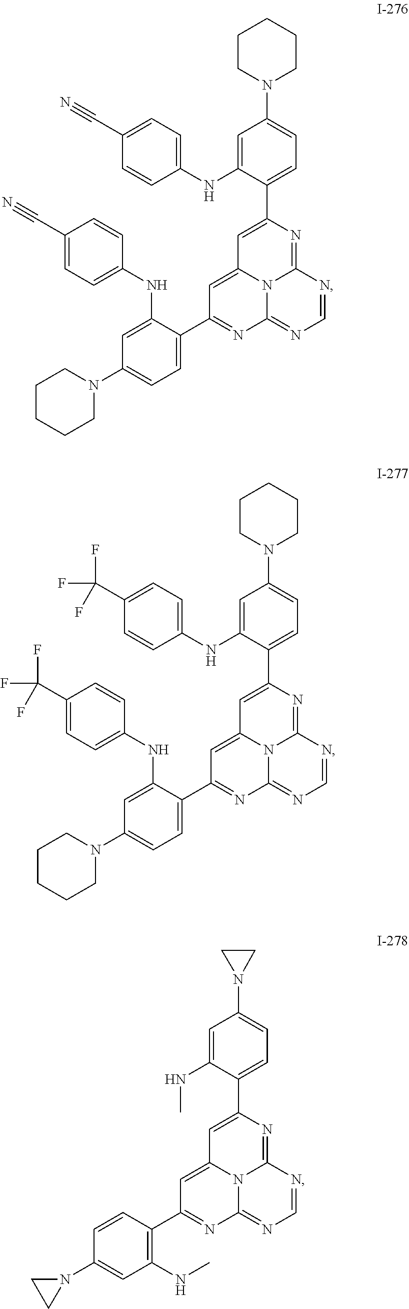 [SMILES: N#Cc1ccc(Nc2cc(N3CCCCC3)ccc2C2=CC3=CC(c4ccc(N5CCCCC5)cc4Nc4ccc(C#N)cc4)=NC4=NC=NC(=N2)N34)cc1 ; FC(F)(F)c1ccc(Nc2cc(N3CCCCC3)ccc2C2=CC3=CC(c4ccc(N5CCCCC5)cc4Nc4ccc(C(F)(F)F)cc4)=NC4=NC=NC(=N2)N34)cc1 ; CNc1cc(N2CC2)ccc1C1=CC2=CC(c3ccc(N4CC4)cc3NC)=NC3=NC=NC(=N1)N23]

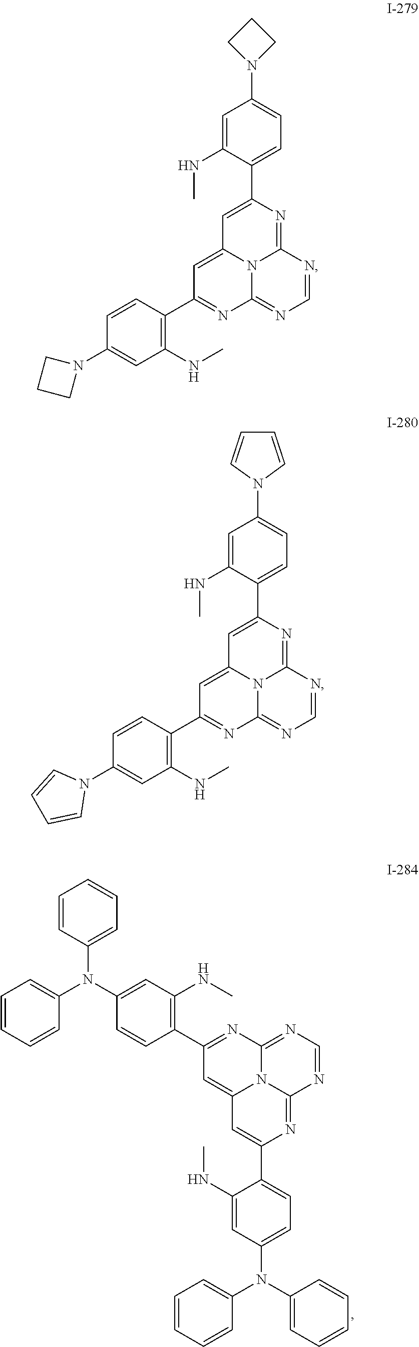 [SMILES: CNc1cc(N2CCC2)ccc1C1=CC2=CC(c3ccc(N4CCC4)cc3NC)=NC3=NC=NC(=N1)N23 ; CNc1cc(-n2cccc2)ccc1C1=CC2=CC(c3ccc(-n4cccc4)cc3NC)=NC3=NC=NC(=N1)N23 ; CNc1cc(N(c2ccccc2)c2ccccc2)ccc1C1=CC2=CC(c3ccc(N(c4ccccc4)c4ccccc4)cc3NC)=NC3=NC=NC(=N1)N23]

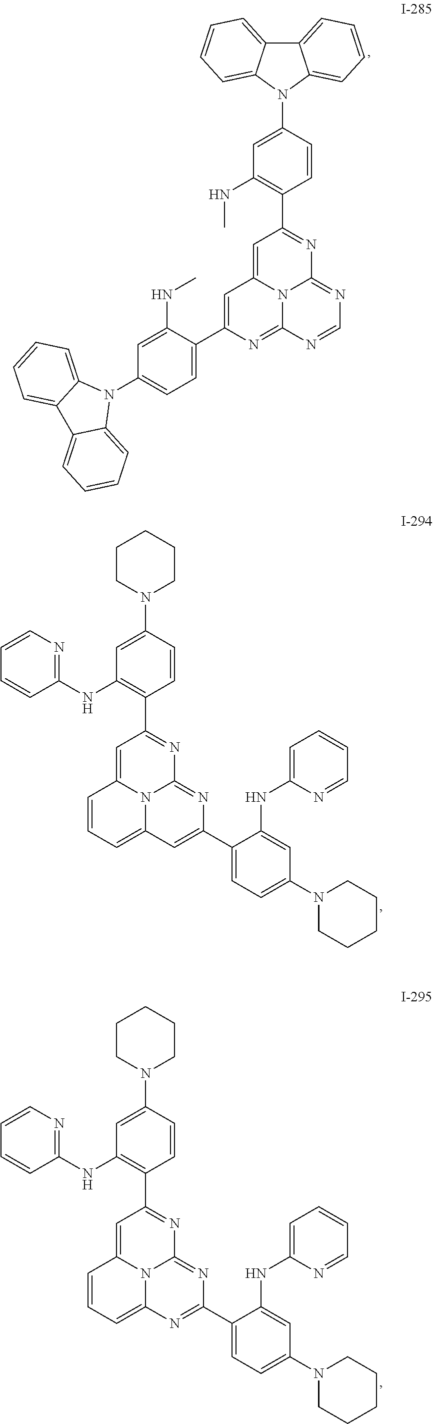 [SMILES: CNc1cc(-n2c3ccccc3c3ccccc32)ccc1C1=CC2=CC(c3ccc(-n4c5ccccc5c5ccccc54)cc3NC)=NC3=NC=NC(=N1)N23 ; C1=CC2=CC(c3ccc(N4CCCCC4)cc3Nc3ccccn3)=NC3=NC(c4ccc(N5CCCCC5)cc4Nc4ccccn4)=CC(=C1)N23 ; C1=CC2=CC(c3ccc(N4CCCCC4)cc3Nc3ccccn3)=NC3=NC(c4ccc(N5CCCCC5)cc4Nc4ccccn4)=NC(=C1)N23]

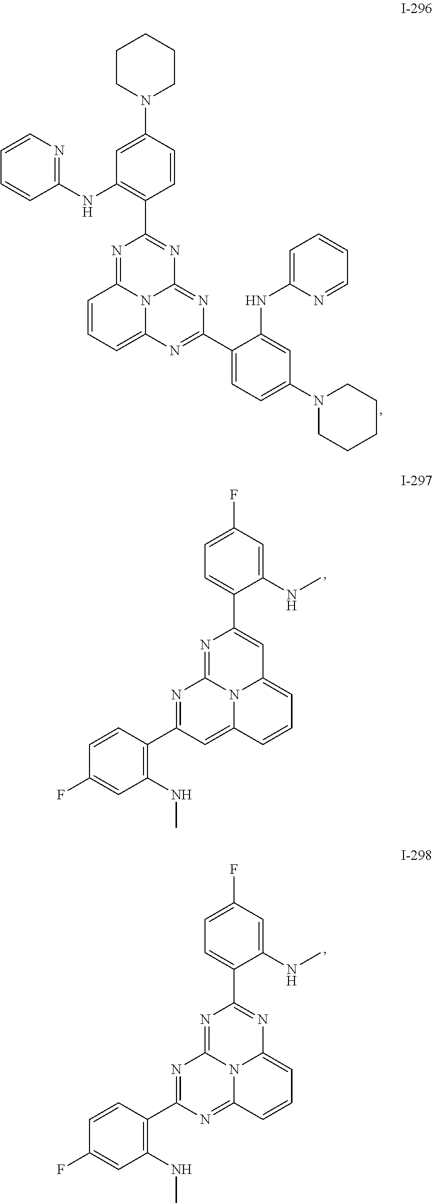 [SMILES: C1=CC2=NC(c3ccc(N4CCCCC4)cc3Nc3ccccn3)=NC3=NC(c4ccc(N5CCCCC5)cc4Nc4ccccn4)=NC(=C1)N23 ; CNc1cc(F)ccc1C1=CC2=CC=CC3=CC(c4ccc(F)cc4NC)=NC(=N1)N23 ; CNc1cc(F)ccc1C1=NC2=CC=CC3=NC(c4ccc(F)cc4NC)=NC(=N1)N23]

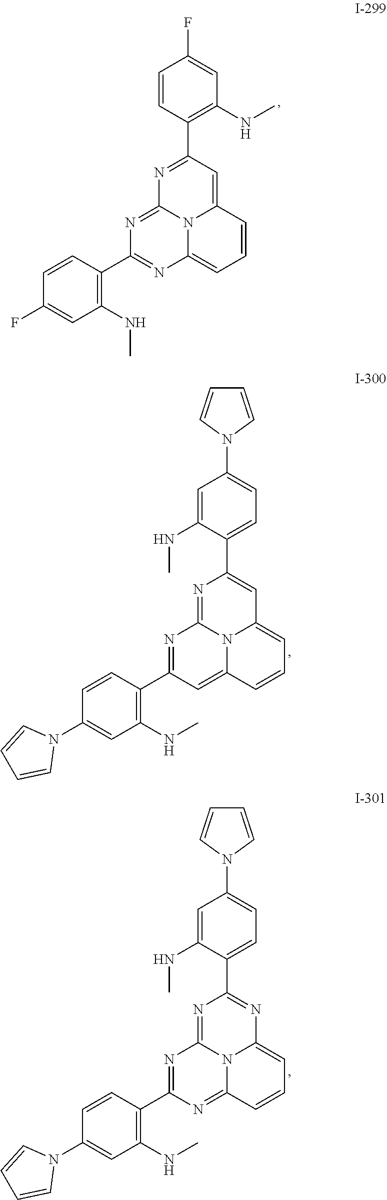 [SMILES: CNc1cc(F)ccc1C1=NC2=NC(c3ccc(F)cc3NC)=NC3=CC=CC(=C1)N32 ; CNc1cc(-n2cccc2)ccc1C1=CC2=CC=CC3=CC(c4ccc(-n5cccc5)cc4NC)=NC(=N1)N23 ; CNc1cc(-n2cccc2)ccc1C1=NC2=CC=CC3=NC(c4ccc(-n5cccc5)cc4NC)=NC(=N1)N23]

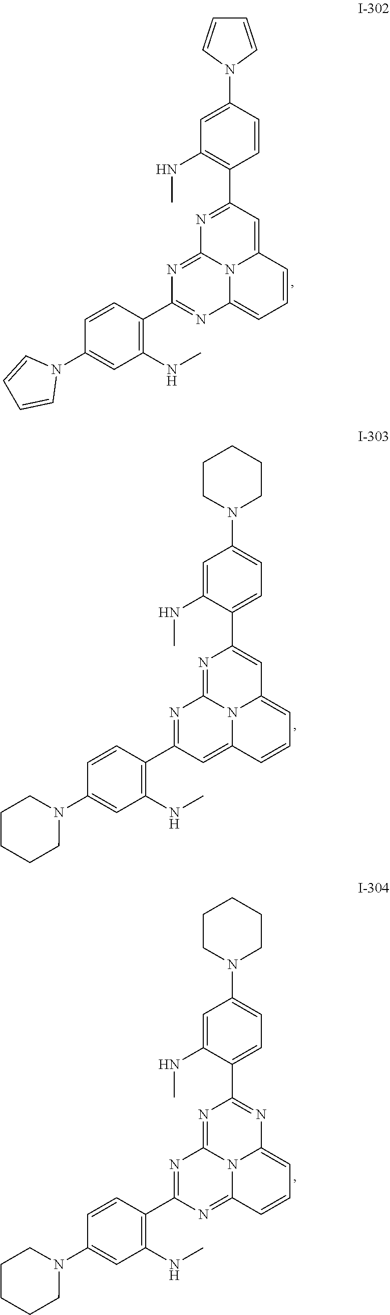 [SMILES: CNc1cc(-n2cccc2)ccc1C1=NC2=NC(c3ccc(-n4cccc4)cc3NC)=NC3=CC=CC(=C1)N32 ; CNc1cc(N2CCCCC2)ccc1C1=CC2=CC=CC3=CC(c4ccc(N5CCCCC5)cc4NC)=NC(=N1)N23 ; CNc1cc(N2CCCCC2)ccc1C1=NC2=CC=CC3=NC(c4ccc(N5CCCCC5)cc4NC)=NC(=N1)N23]

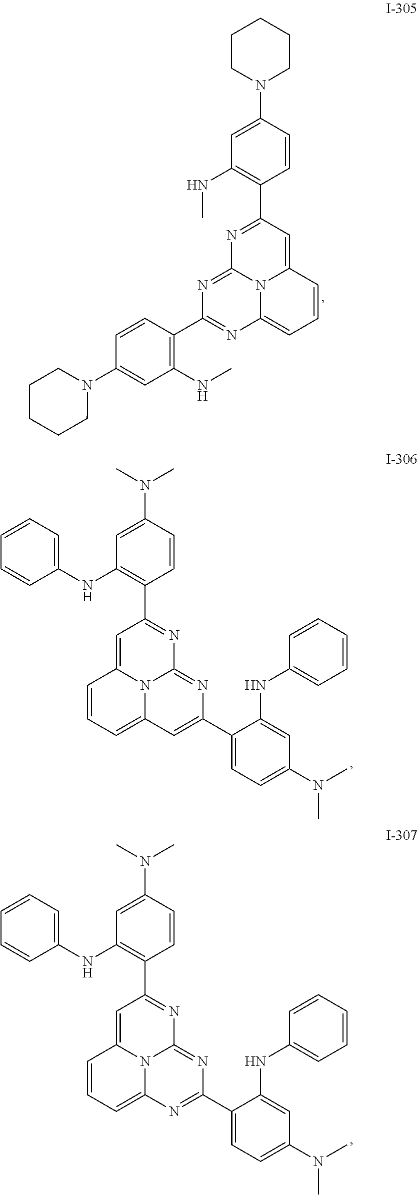 [SMILES: CNc1cc(N2CCCCC2)ccc1C1=NC2=NC(c3ccc(N4CCCCC4)cc3NC)=NC3=CC=CC(=C1)N32 ; CN(C)c1ccc(C2=CC3=CC=CC4=CC(c5ccc(N(C)C)cc5Nc5ccccc5)=NC(=N2)N34)c(Nc2ccccc2)c1 ; CN(C)c1ccc(C2=NC3=NC(c4ccc(N(C)C)cc4Nc4ccccc4)=NC4=CC=CC(=C2)N43)c(Nc2ccccc2)c1]

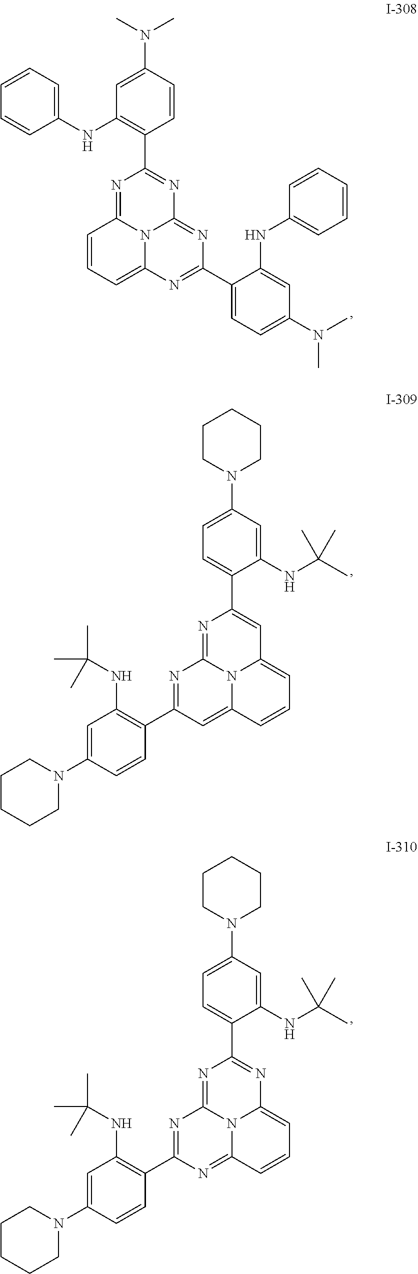 [SMILES: CN(C)c1ccc(C2=NC3=CC=CC4=NC(c5ccc(N(C)C)cc5Nc5ccccc5)=NC(=N2)N34)c(Nc2ccccc2)c1 ; CC(C)(C)Nc1cc(N2CCCCC2)ccc1C1=CC2=CC=CC3=CC(c4ccc(N5CCCCC5)cc4NC(C)(C)C)=NC(=N1)N23 ; CC(C)(C)Nc1cc(N2CCCCC2)ccc1C1=NC2=CC=CC3=NC(c4ccc(N5CCCCC5)cc4NC(C)(C)C)=NC(=N1)N23]

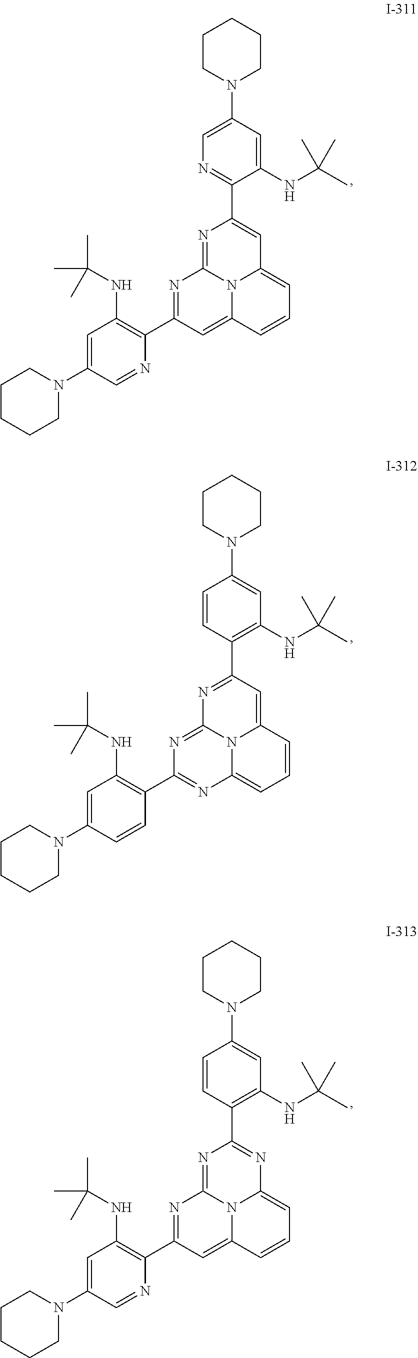 [SMILES: CC(C)(C)Nc1cc(N2CCCCC2)cnc1C1=CC2=CC=CC3=CC(c4ncc(N5CCCCC5)cc4NC(C)(C)C)=NC(=N1)N23 ; CC(C)(C)Nc1cc(N2CCCCC2)ccc1C1=NC2=NC(c3ccc(N4CCCCC4)cc3NC(C)(C)C)=NC3=CC=CC(=C1)N32 ; CC(C)(C)Nc1cc(N2CCCCC2)ccc1C1=NC2=CC=CC3=CC(c4ncc(N5CCCCC5)cc4NC(C)(C)C)=NC(=N1)N32]

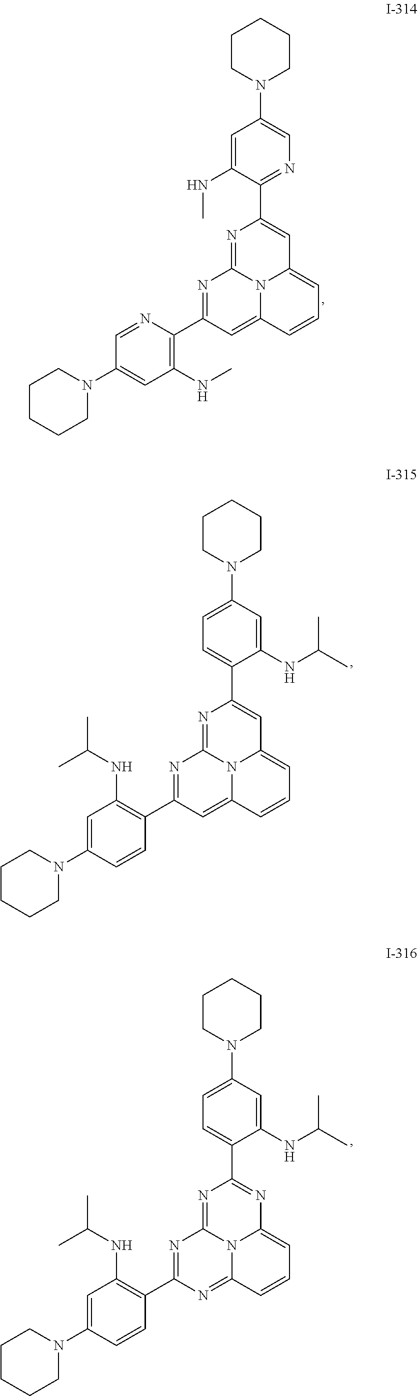 [SMILES: CNc1cc(N2CCCCC2)cnc1C1=CC2=CC=CC3=CC(c4ncc(N5CCCCC5)cc4NC)=NC(=N1)N23 ; CC(C)Nc1cc(N2CCCCC2)ccc1C1=CC2=CC=CC3=CC(c4ccc(N5CCCCC5)cc4NC(C)C)=NC(=N1)N23 ; CC(C)Nc1cc(N2CCCCC2)ccc1C1=NC2=CC=CC3=NC(c4ccc(N5CCCCC5)cc4NC(C)C)=NC(=N1)N23]

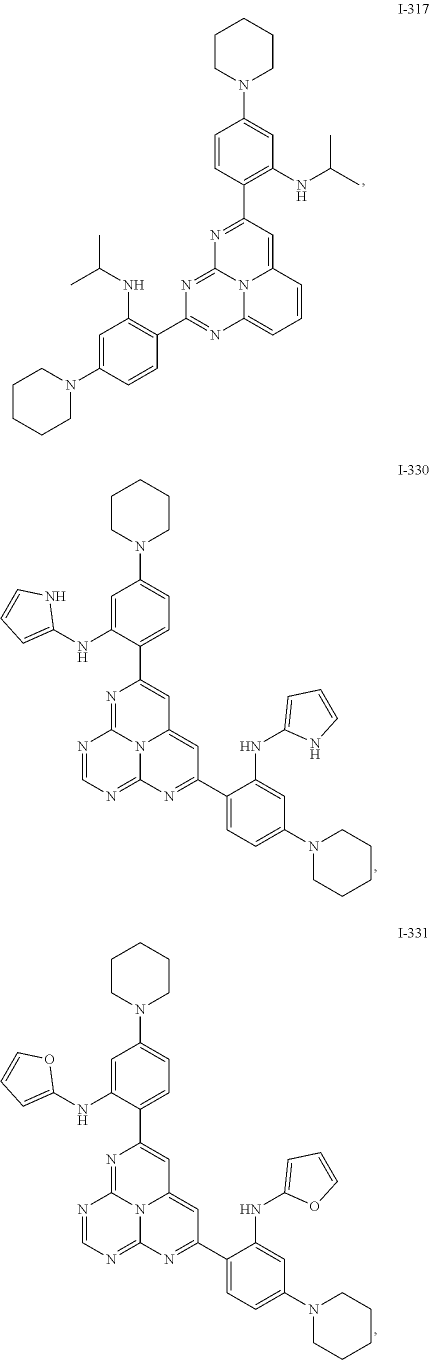 [SMILES: CC(C)Nc1cc(N2CCCCC2)ccc1C1=NC2=NC(c3ccc(N4CCCCC4)cc3NC(C)C)=NC3=CC=CC(=C1)N32 ; C1=NC2=NC(c3ccc(N4CCCCC4)cc3Nc3ccc[nH]3)=CC3=CC(c4ccc(N5CCCCC5)cc4Nc4ccc[nH]4)=NC(=N1)N32 ; C1=NC2=NC(c3ccc(N4CCCCC4)cc3Nc3ccco3)=CC3=CC(c4ccc(N5CCCCC5)cc4Nc4ccco4)=NC(=N1)N32]

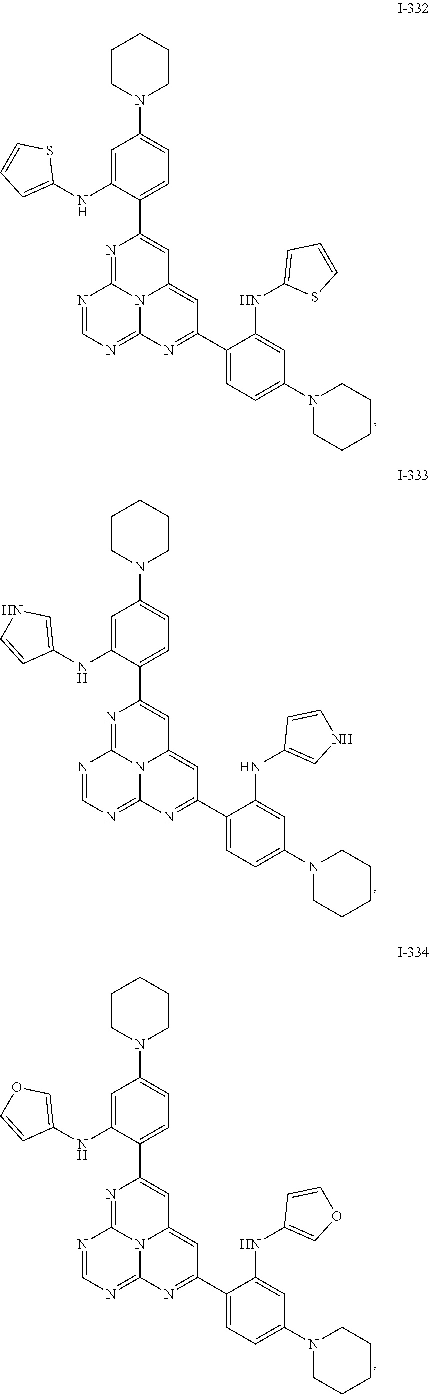 [SMILES: C1=NC2=NC(c3ccc(N4CCCCC4)cc3Nc3cccs3)=CC3=CC(c4ccc(N5CCCCC5)cc4Nc4cccs4)=NC(=N1)N32 ; C1=NC2=NC(c3ccc(N4CCCCC4)cc3Nc3cc[nH]c3)=CC3=CC(c4ccc(N5CCCCC5)cc4Nc4cc[nH]c4)=NC(=N1)N32 ; C1=NC2=NC(c3ccc(N4CCCCC4)cc3Nc3ccoc3)=CC3=CC(c4ccc(N5CCCCC5)cc4Nc4ccoc4)=NC(=N1)N32]

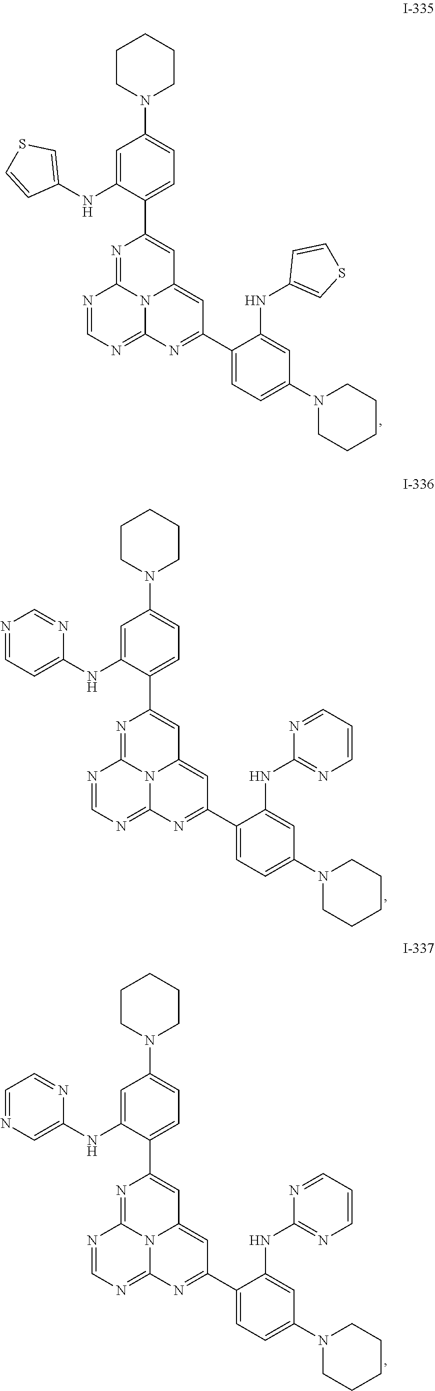 [SMILES: C1=NC2=NC(c3ccc(N4CCCCC4)cc3Nc3ccsc3)=CC3=CC(c4ccc(N5CCCCC5)cc4Nc4ccsc4)=NC(=N1)N32 ; C1=NC2=NC(c3ccc(N4CCCCC4)cc3Nc3ccncn3)=CC3=CC(c4ccc(N5CCCCC5)cc4Nc4ncccn4)=NC(=N1)N32 ; C1=NC2=NC(c3ccc(N4CCCCC4)cc3Nc3cnccn3)=CC3=CC(c4ccc(N5CCCCC5)cc4Nc4ncccn4)=NC(=N1)N32]

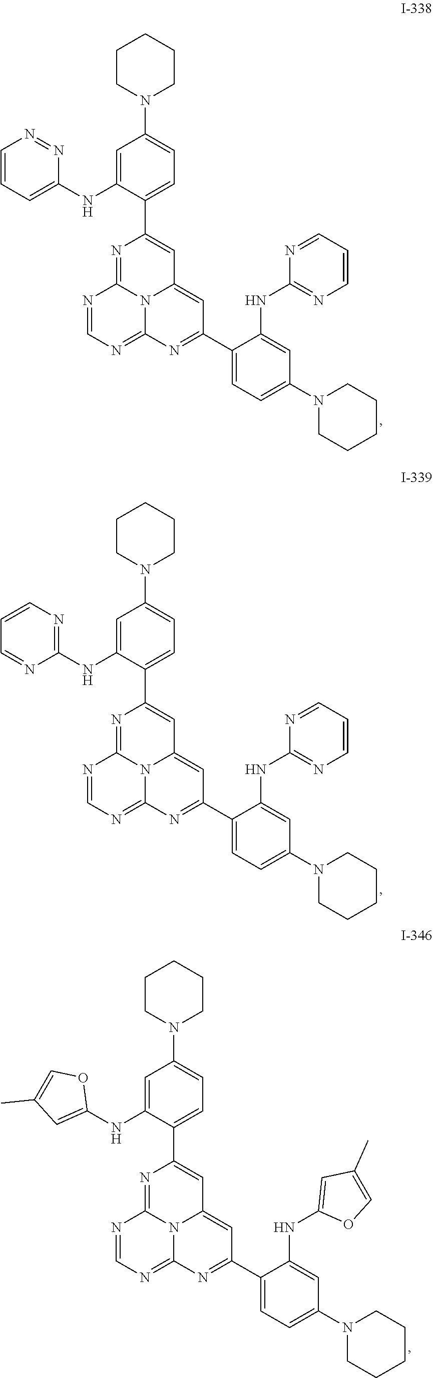 [SMILES: C1=NC2=NC(c3ccc(N4CCCCC4)cc3Nc3cccnn3)=CC3=CC(c4ccc(N5CCCCC5)cc4Nc4ncccn4)=NC(=N1)N32 ; C1=NC2=NC(c3ccc(N4CCCCC4)cc3Nc3ncccn3)=CC3=CC(c4ccc(N5CCCCC5)cc4Nc4ncccn4)=NC(=N1)N32 ; Cc1coc(Nc2cc(N3CCCCC3)ccc2C2=CC3=CC(c4ccc(N5CCCCC5)cc4Nc4cc(C)co4)=NC4=NC=NC(=N2)N34)c1]

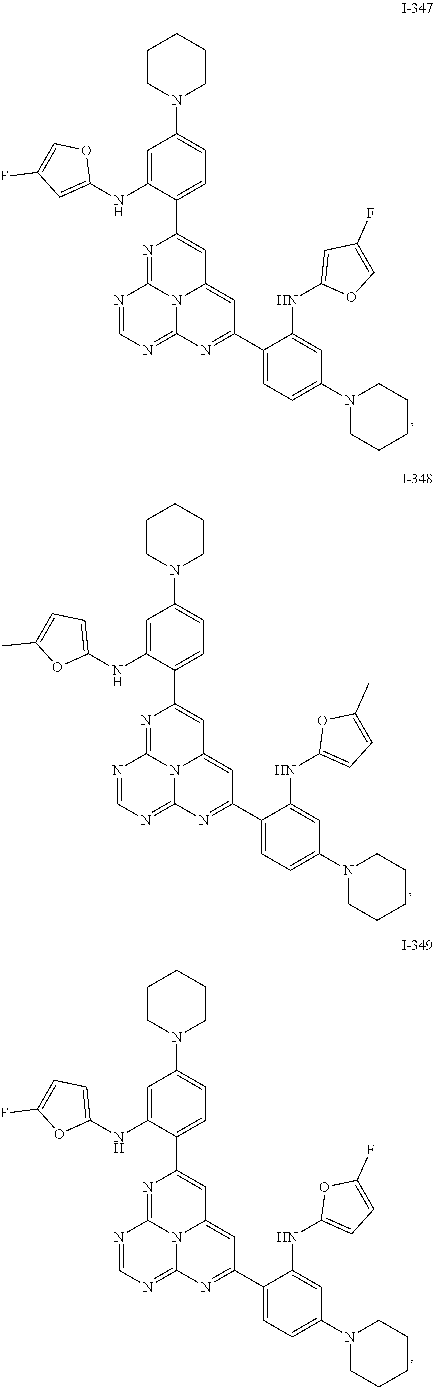 [SMILES: Fc1coc(Nc2cc(N3CCCCC3)ccc2C2=CC3=CC(c4ccc(N5CCCCC5)cc4Nc4cc(F)co4)=NC4=NC=NC(=N2)N34)c1 ; Cc1ccc(Nc2cc(N3CCCCC3)ccc2C2=CC3=CC(c4ccc(N5CCCCC5)cc4Nc4ccc(C)o4)=NC4=NC=NC(=N2)N34)o1 ; Fc1ccc(Nc2cc(N3CCCCC3)ccc2C2=CC3=CC(c4ccc(N5CCCCC5)cc4Nc4ccc(F)o4)=NC4=NC=NC(=N2)N34)o1]

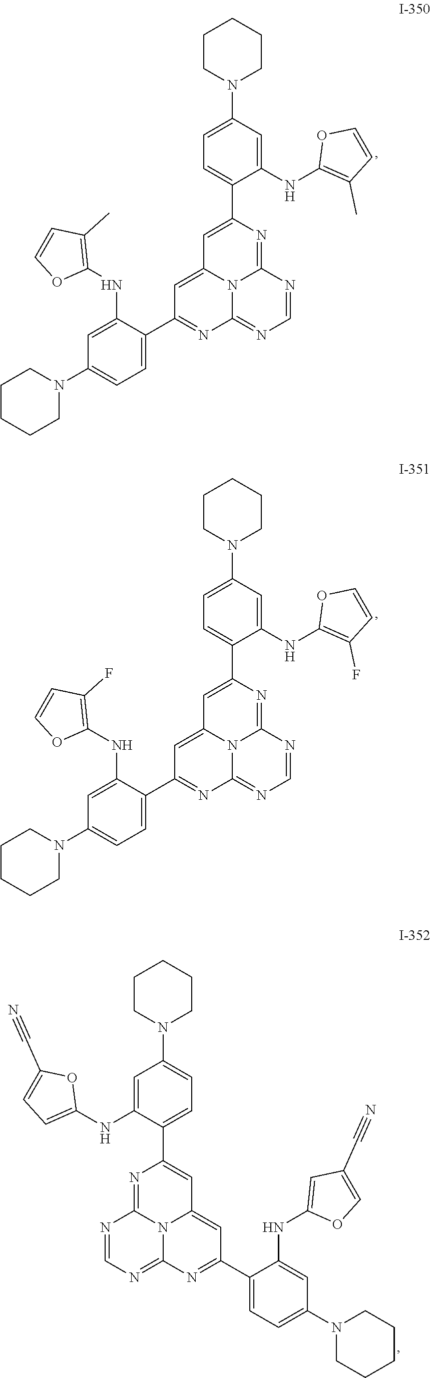 [SMILES: Cc1ccoc1Nc1cc(N2CCCCC2)ccc1C1=CC2=CC(c3ccc(N4CCCCC4)cc3Nc3occc3C)=NC3=NC=NC(=N1)N23 ; Fc1ccoc1Nc1cc(N2CCCCC2)ccc1C1=CC2=CC(c3ccc(N4CCCCC4)cc3Nc3occc3F)=NC3=NC=NC(=N1)N23 ; N#Cc1coc(Nc2cc(N3CCCCC3)ccc2C2=NC3=NC=NC4=NC(c5ccc(N6CCCCC6)cc5Nc5ccc(C#N)o5)=CC(=C2)N34)c1]

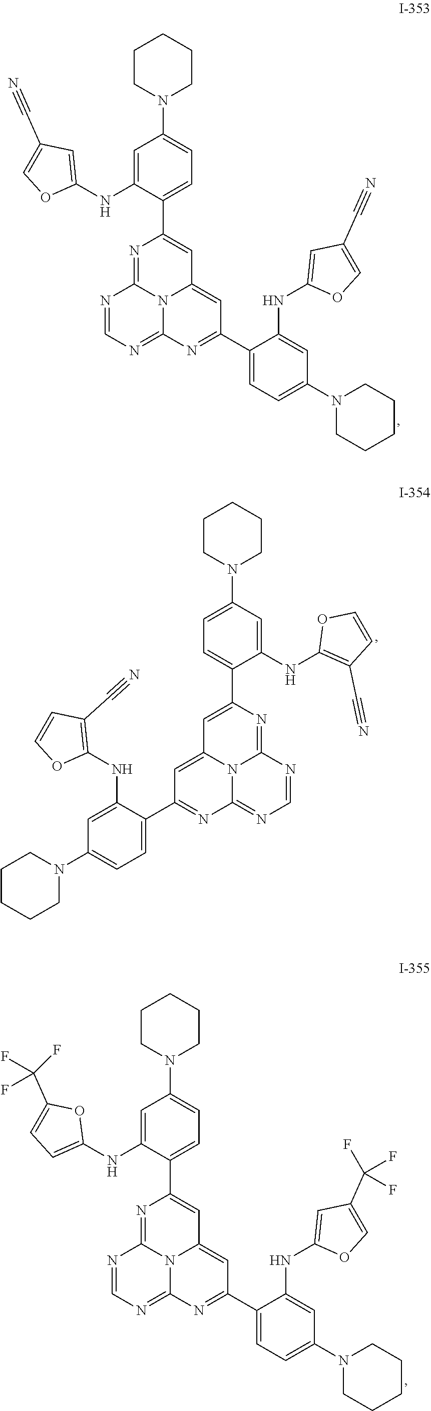 [SMILES: N#Cc1coc(Nc2cc(N3CCCCC3)ccc2C2=CC3=CC(c4ccc(N5CCCCC5)cc4Nc4cc(C#N)co4)=NC4=NC=NC(=N2)N34)c1 ; N#Cc1ccoc1Nc1cc(N2CCCCC2)ccc1C1=CC2=CC(c3ccc(N4CCCCC4)cc3Nc3occc3C#N)=NC3=NC=NC(=N1)N23 ; FC(F)(F)c1coc(Nc2cc(N3CCCCC3)ccc2C2=NC3=NC=NC4=NC(c5ccc(N6CCCCC6)cc5Nc5ccc(C(F)(F)F)o5)=CC(=C2)N34)c1]

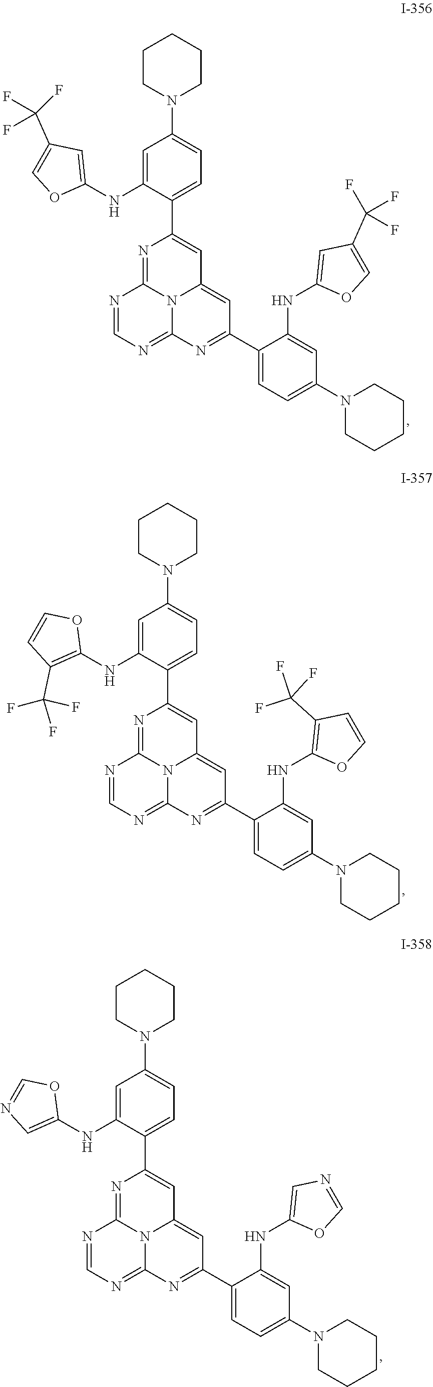 [SMILES: FC(F)(F)c1coc(Nc2cc(N3CCCCC3)ccc2C2=CC3=CC(c4ccc(N5CCCCC5)cc4Nc4cc(C(F)(F)F)co4)=NC4=NC=NC(=N2)N34)c1 ; FC(F)(F)c1ccoc1Nc1cc(N2CCCCC2)ccc1C1=CC2=CC(c3ccc(N4CCCCC4)cc3Nc3occc3C(F)(F)F)=NC3=NC=NC(=N1)N23 ; C1=NC2=NC(c3ccc(N4CCCCC4)cc3Nc3cnco3)=CC3=CC(c4ccc(N5CCCCC5)cc4Nc4cnco4)=NC(=N1)N32]

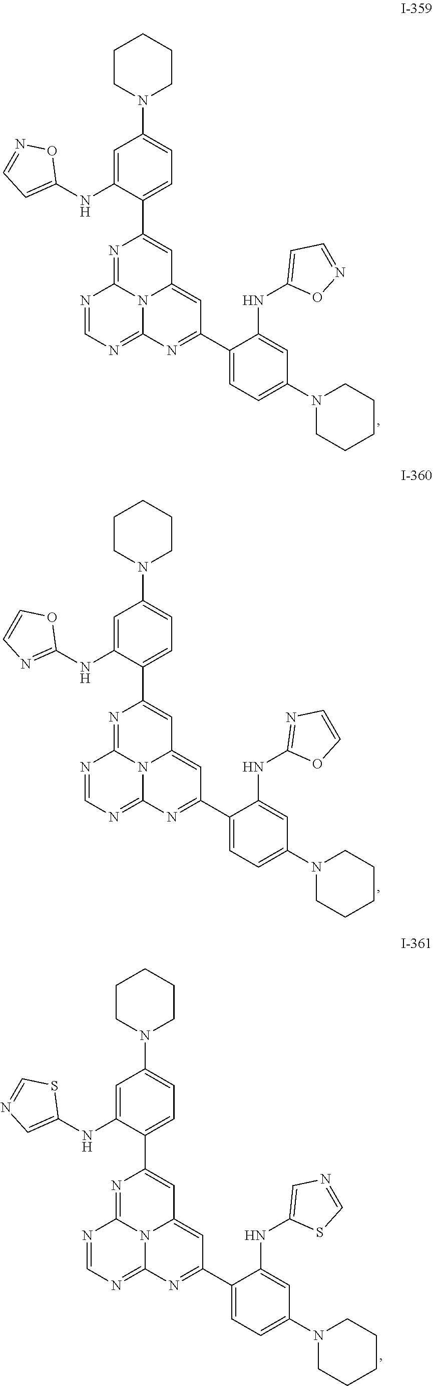 [SMILES: C1=NC2=NC(c3ccc(N4CCCCC4)cc3Nc3ccno3)=CC3=CC(c4ccc(N5CCCCC5)cc4Nc4ccno4)=NC(=N1)N32 ; C1=NC2=NC(c3ccc(N4CCCCC4)cc3Nc3ncco3)=CC3=CC(c4ccc(N5CCCCC5)cc4Nc4ncco4)=NC(=N1)N32 ; C1=NC2=NC(c3ccc(N4CCCCC4)cc3Nc3cncs3)=CC3=CC(c4ccc(N5CCCCC5)cc4Nc4cncs4)=NC(=N1)N32]

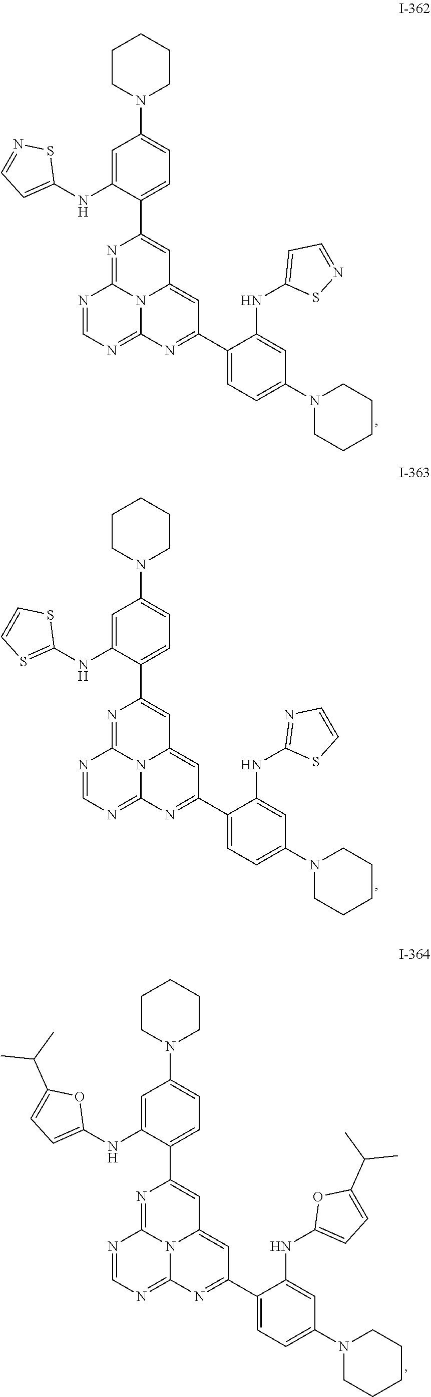

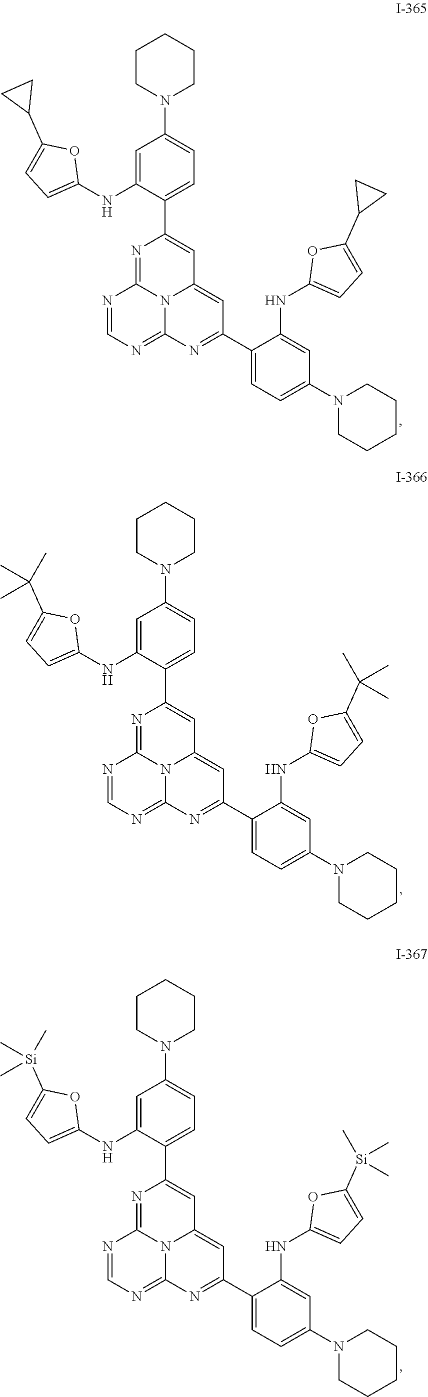 [SMILES: C1=NC2=NC(c3ccc(N4CCCCC4)cc3Nc3ccc(C4CC4)o3)=CC3=CC(c4ccc(N5CCCCC5)cc4Nc4ccc(C5CC5)o4)=NC(=N1)N32 ; CC(C)(C)c1ccc(Nc2cc(N3CCCCC3)ccc2C2=CC3=CC(c4ccc(N5CCCCC5)cc4Nc4ccc(C(C)(C)C)o4)=NC4=NC=NC(=N2)N34)o1 ; C[Si](C)(C)c1ccc(Nc2cc(N3CCCCC3)ccc2C2=CC3=CC(c4ccc(N5CCCCC5)cc4Nc4ccc([Si](C)(C)C)o4)=NC4=NC=NC(=N2)N34)o1]

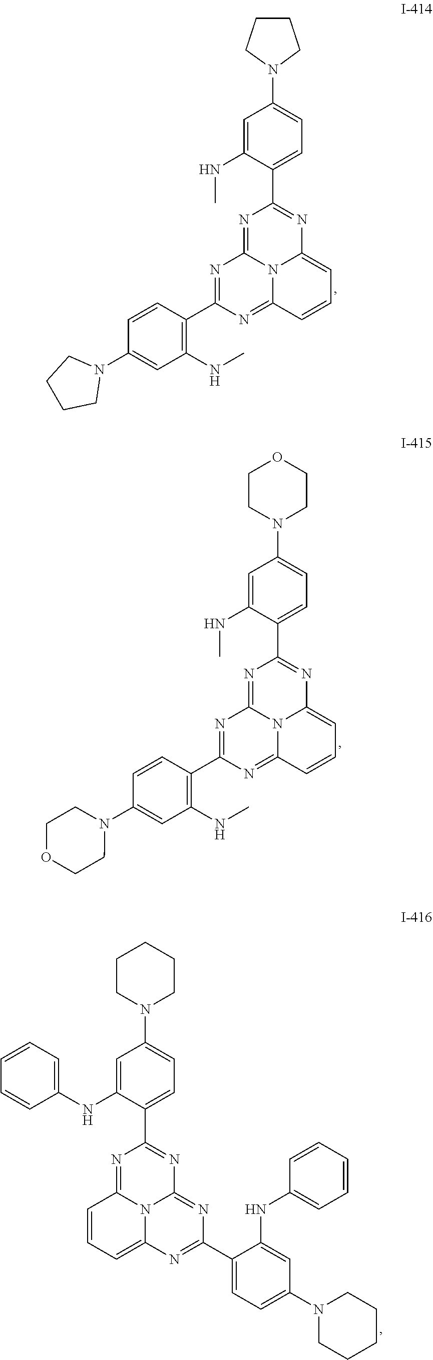 [SMILES: CNc1cc(N2CCCC2)ccc1C1=NC2=CC=CC3=NC(c4ccc(N5CCCC5)cc4NC)=NC(=N1)N23 ; CNc1cc(N2CCOCC2)ccc1C1=NC2=CC=CC3=NC(c4ccc(N5CCOCC5)cc4NC)=NC(=N1)N23 ; C1=CC2=NC(c3ccc(N4CCCCC4)cc3Nc3ccccc3)=NC3=NC(c4ccc(N5CCCCC5)cc4Nc4ccccc4)=NC(=C1)N23]

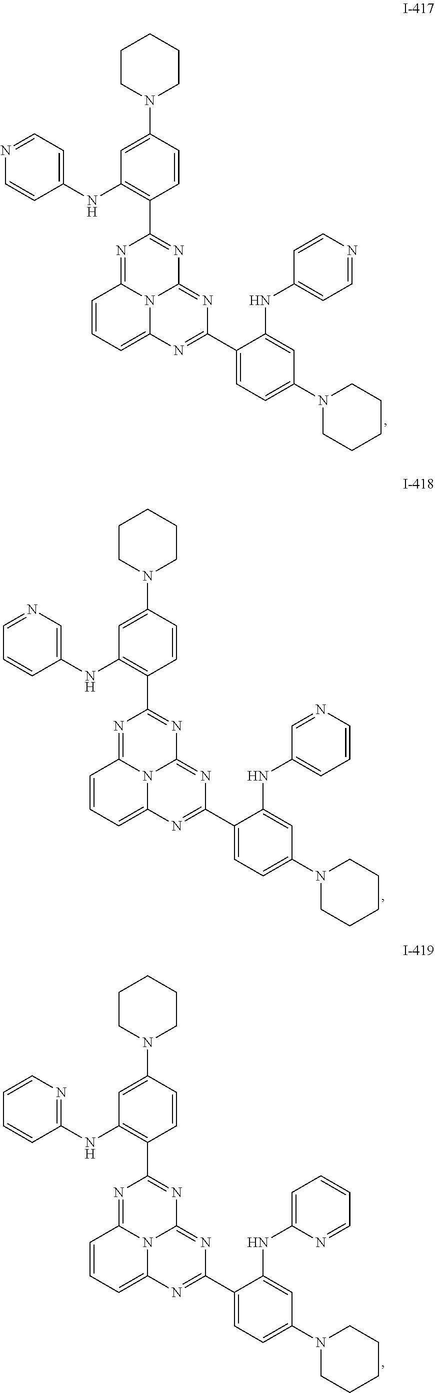 [SMILES: C1=CC2=NC(c3ccc(N4CCCCC4)cc3Nc3ccncc3)=NC3=NC(c4ccc(N5CCCCC5)cc4Nc4ccncc4)=NC(=C1)N23 ; C1=CC2=NC(c3ccc(N4CCCCC4)cc3Nc3cccnc3)=NC3=NC(c4ccc(N5CCCCC5)cc4Nc4cccnc4)=NC(=C1)N23 ; C1=CC2=NC(c3ccc(N4CCCCC4)cc3Nc3ccccn3)=NC3=NC(c4ccc(N5CCCCC5)cc4Nc4ccccn4)=NC(=C1)N23]

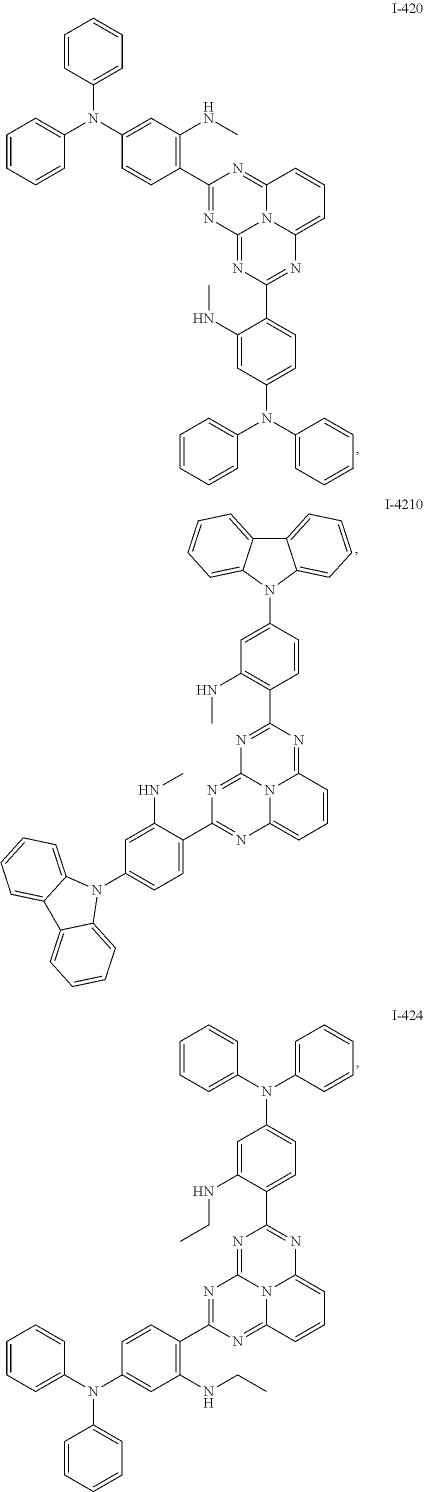

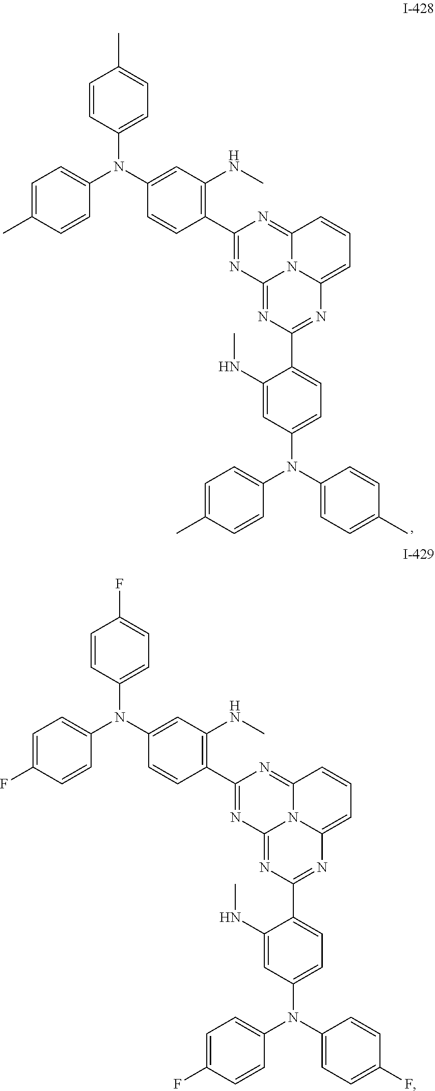

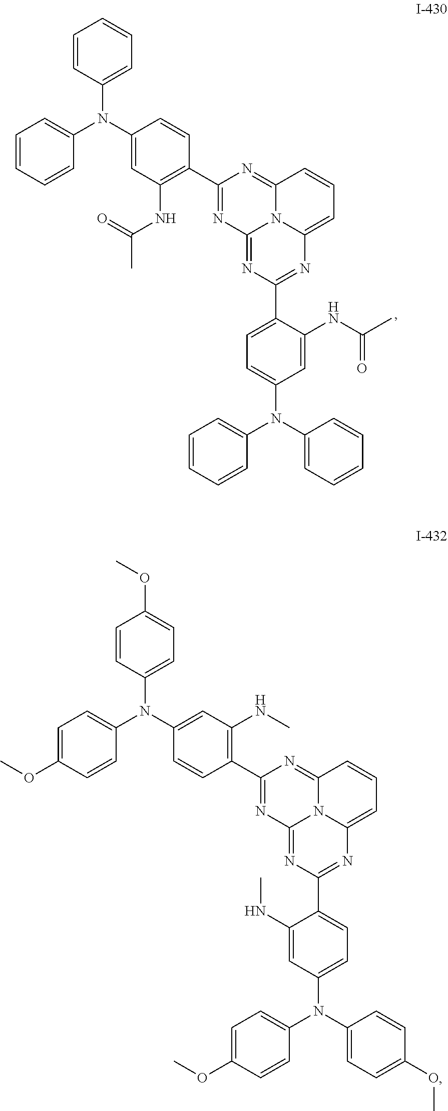

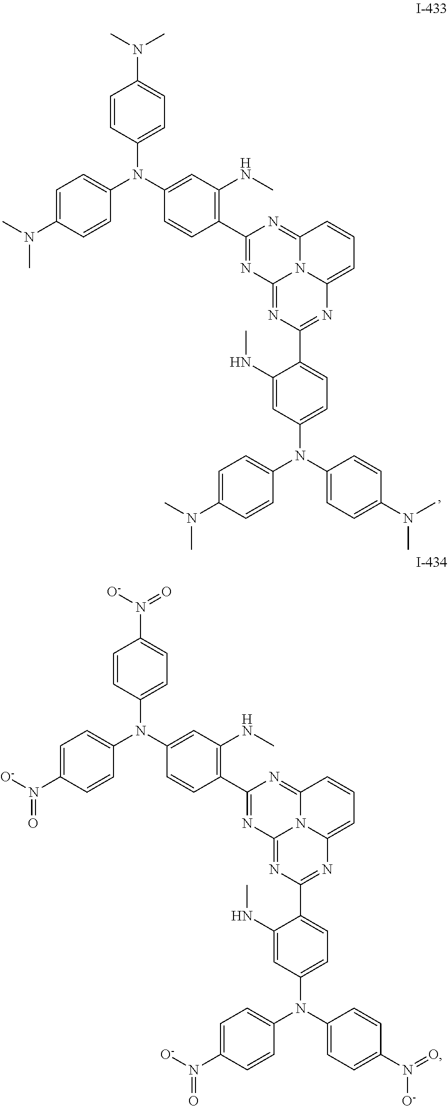

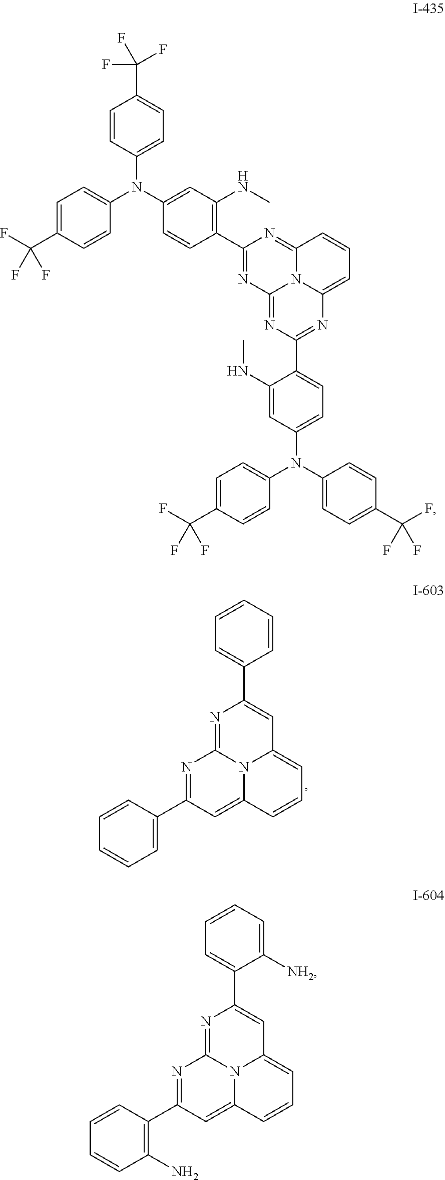 [SMILES: CNc1cc(N(c2ccc(C(F)(F)F)cc2)c2ccc(C(F)(F)F)cc2)ccc1C1=NC2=CC=CC3=NC(c4ccc(N(c5ccc(C(F)(F)F)cc5)c5ccc(C(F)(F)F)cc5)cc4NC)=NC(=N1)N23 ; C1=CC2=CC(c3ccccc3)=NC3=NC(c4ccccc4)=CC(=C1)N23 ; Nc1ccccc1C1=CC2=CC=CC3=CC(c4ccccc4N)=NC(=N1)N23]

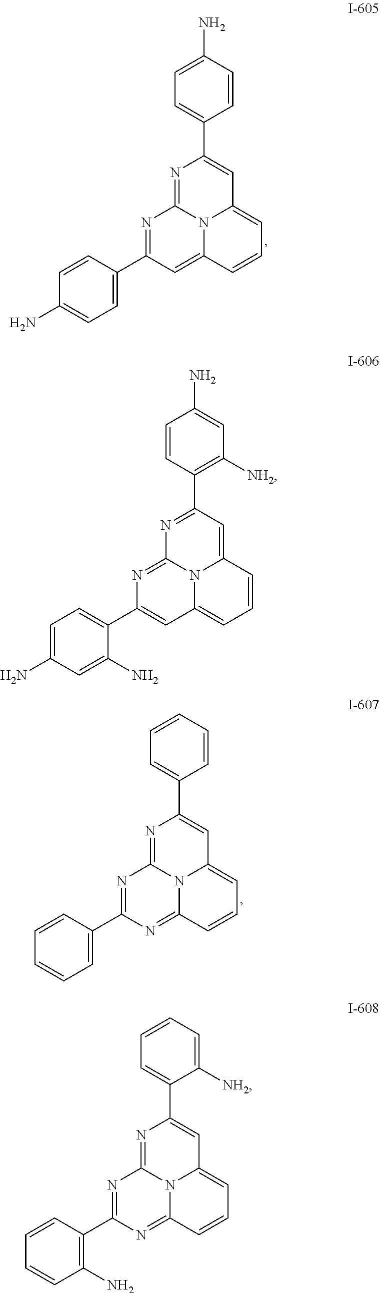 [SMILES: Nc1ccc(C2=CC3=CC=CC4=CC(c5ccc(N)cc5)=NC(=N2)N34)cc1 ; Nc1ccc(C2=CC3=CC=CC4=CC(c5ccc(N)cc5N)=NC(=N2)N34)c(N)c1 ; C1=CC2=NC(c3ccccc3)=NC3=NC(c4ccccc4)=CC(=C1)N23 ; Nc1ccccc1C1=CC2=CC=CC3=NC(c4ccccc4N)=NC(=N1)N23]

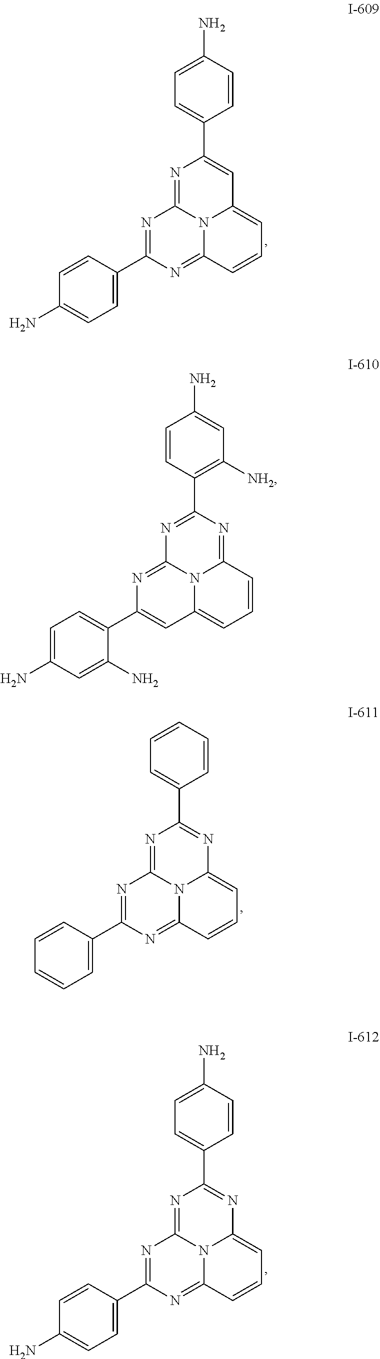 [SMILES: Nc1ccc(C2=CC3=CC=CC4=NC(c5ccc(N)cc5)=NC(=N2)N34)cc1 ; Nc1ccc(C2=CC3=CC=CC4=NC(c5ccc(N)cc5N)=NC(=N2)N34)c(N)c1 ; C1=CC2=NC(c3ccccc3)=NC3=NC(c4ccccc4)=NC(=C1)N23 ; Nc1ccc(C2=NC3=CC=CC4=NC(c5ccc(N)cc5)=NC(=N2)N34)cc1]

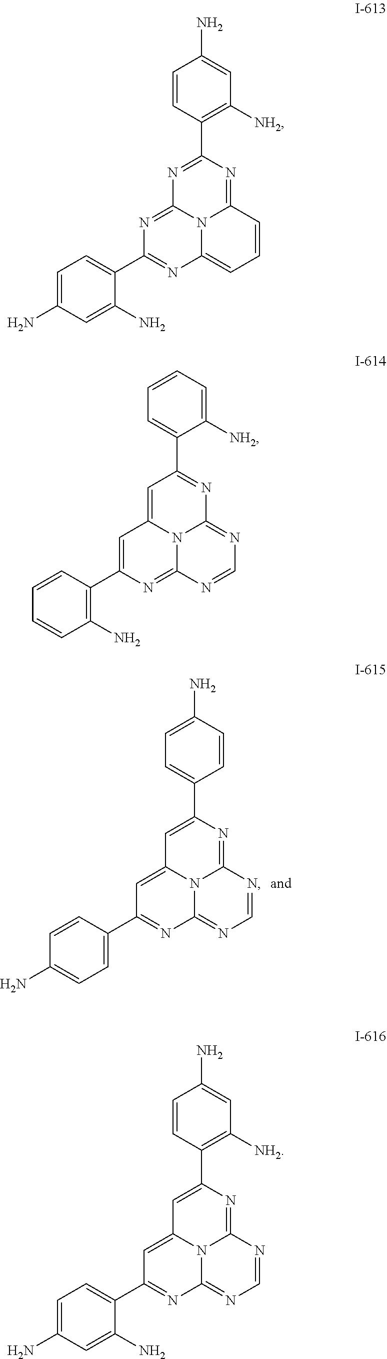 [SMILES: Nc1ccc(C2=NC3=CC=CC4=NC(c5ccc(N)cc5N)=NC(=N2)N34)c(N)c1 ; Nc1ccccc1C1=CC2=CC(c3ccccc3N)=NC3=NC=NC(=N1)N23 ; Nc1ccc(C2=CC3=CC(c4ccc(N)cc4)=NC4=NC=NC(=N2)N34)cc1 ; Nc1ccc(C2=CC3=CC(c4ccc(N)cc4N)=NC4=NC=NC(=N2)N34)c(N)c1]

In another aspect, the present application includes an organic light-emitting diode comprising at least one compound of the present application.

In another aspect, the present application includes a photocatalyst comprising at least one compound of the present application.

In another aspect, the present application includes a triplet quencher comprising at least one compound of the present application.

### III. Methods of Preparing the Compounds of the Application

Compounds of the present application can be prepared by various synthetic processes. The choice of particular structural features and/or substituents may influence the selection of one process over another. The selection of a particular process to prepare a given compound of Formula I is within the purview of the person of skill in the art. Some starting materials for preparing compounds of the present application are available from commercial chemical sources. Other starting materials, for example as described below, are readily prepared from available precursors using straightforward transformations that are well known in the art. In the Schemes below showing the preparation of compounds of the application, all variables are as defined in Formula I, unless otherwise stated.

The compounds of Formula I generally can be prepared according to the processes illustrated in the Schemes below. In the structural formulae shown below the variables are as defined in Formula I unless otherwise stated. A person skilled in the art would appreciate that many of the reactions depicted in the Schemes below would be sensitive to oxygen and water and would know to perform the reaction under an anhydrous, inert atmosphere if needed. Reaction temperatures and times are presented for illustrative purposes only and may be varied to optimize yield as would be understood by a person skilled in the art.

Accordingly, in some embodiments, the compounds of the present application can be prepared as shown in the retrosynthetic Schemes below. The term “Hal” as used in the Schemes refers to halogen. For example, it can refer to Br, Cl, or I. Each R^{e} is independently selected from C₁₋₃alkyl.

Accordingly, in some embodiments, certain compounds of Formula I (shown as compound of Formula A, wherein X¹ and X⁶ are CR⁴ and CR⁹, respectively, and X², X³, X⁴ and X⁵ are N) are prepared as shown in retrosynthetic Scheme 1. Therefore, 2,6-diaminopyridine compound D can react as a nucleophile with the acyl halide compounds of Formulae E and F to provide intermediate compound of Formula B. Intermediate compound of Formula B can produce compound A through cyclization with cyanamide C.

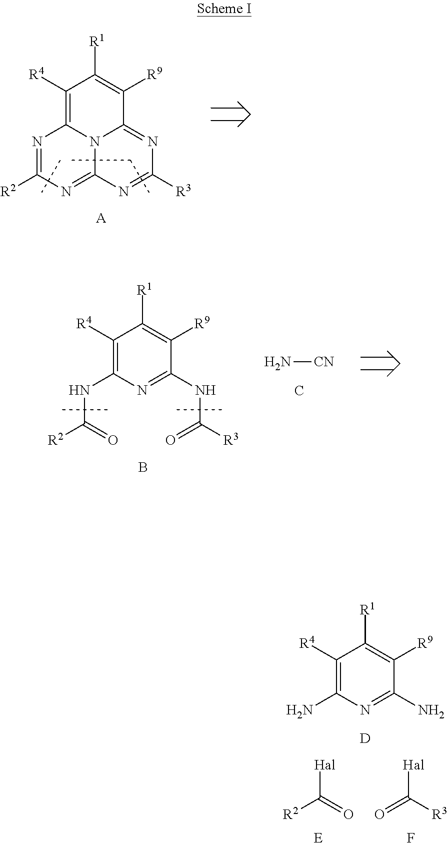

In some embodiments, the certain compounds of Formula I (shown as compound of Formula G, wherein X¹, X², X⁵ and X⁶ are CR⁴, CR⁵, CR⁸ and CR⁹, respectively, and X³ and X⁴ are N) are prepared as shown in retrosynthetic Scheme II. Therefore, the carbonyl compounds of Formulae K and L can undergo an aromatic nucleophilic substitution with the dihalopyridine compound of Formula J to provide the intermediate compound of Formula H. The intermediate compound of Formula H can cyclize with cyanamide of Formula C to produce the compound of Formula G.

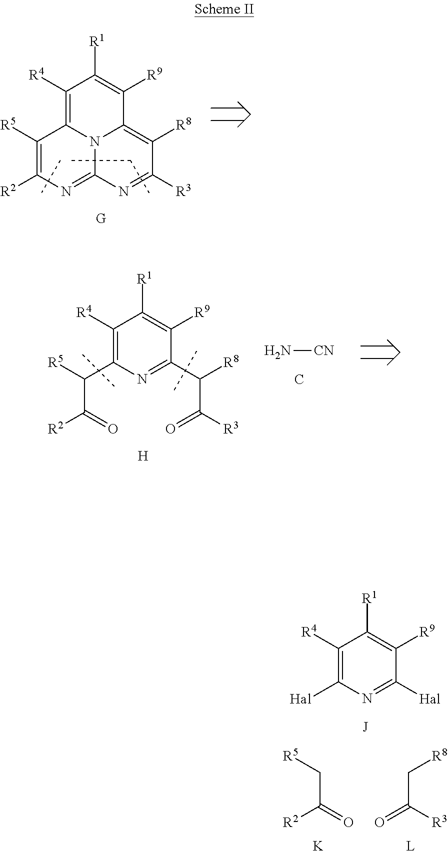

In some embodiments, the certain compounds of Formula I (shown as compound of Formula G, wherein X¹, X², X⁵ and X⁶ are CR⁴, CR⁵, CR⁸ and CR⁹, respectively, and X³ and X⁴ are N) are prepared as shown in retrosynthetic Scheme Ill. Therefore, the compounds of Formulae N and O can undergo cyclization with the compound of Formula M to produce the compound of Formula G.

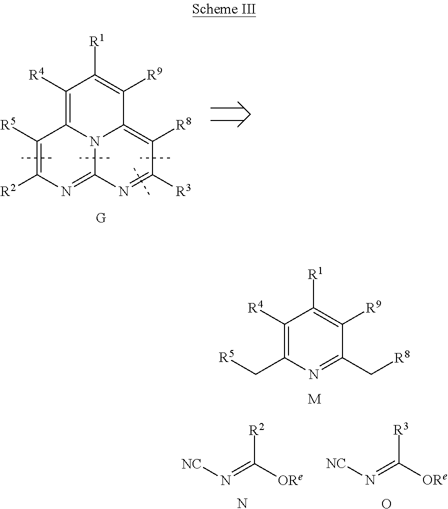

In some embodiments, certain compounds of Formula I (shown as compound of Formula P, wherein X¹, X² and X⁶ are CR⁴, CR⁵ and CR⁹, respectively, and X³, X⁴ and X⁵ are N) are prepared as shown in retrosynthetic Scheme IV. Therefore, the compounds of Formulae N and O can undergo cyclization with the aminopyridine compound of Formula Q to produce the compound of Formula P.

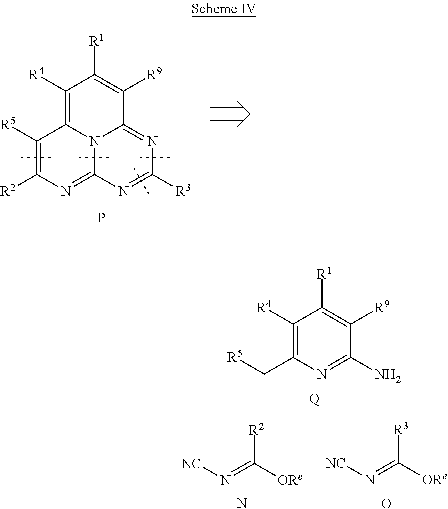

In some embodiments, certain compounds of Formula I (shown as compound of Formula P, wherein X¹, X² and X⁶ are CR⁴, CR⁵ and CR⁹, respectively, and X³, X⁴ and X⁵ are N) are prepared as shown in retrosynthetic Scheme V. Therefore, the acyl halide compound of Formula F can react with the halogenated aminopyridine compound of Formula T to obtain the intermediate compound of Formula S. The intermediate compound of Formula S can undergo aromatic nucleophilic substitution with the carbonyl compound of Formula K to produce the intermediate compound of Formula R. The intermediate compound of Formula R can then cyclize with cyanamide of Formula C to obtain the compound for Formula P.

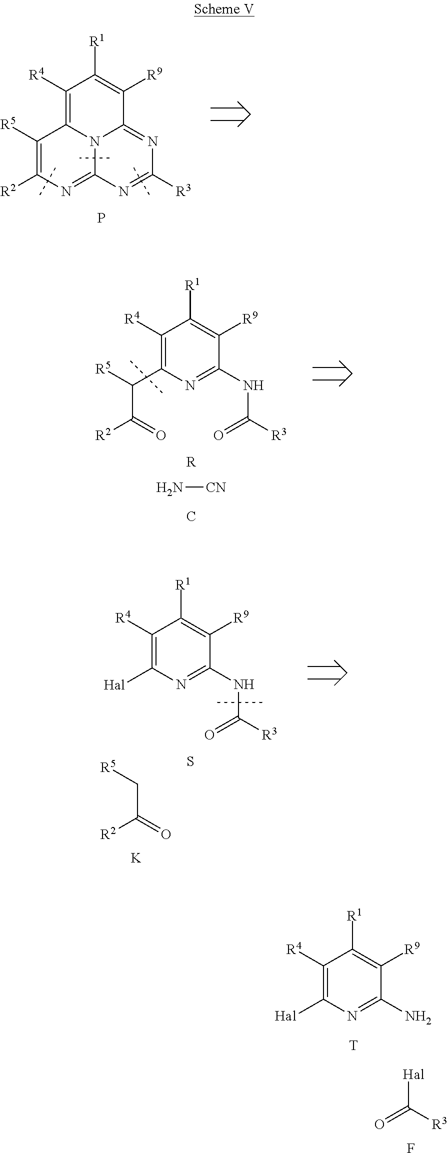

In some embodiments, certain compounds of Formula I (shown as compound of Formula U, wherein X¹ and X² are CR⁴ and CR⁵, respectively, and X³, X⁴, X⁵ and X⁶ are N) are prepared as shown in retrosynthetic Scheme VI. Therefore, the acyl halide compound of Formula F can react with the halogenated aminopyrimidine compound of Formula X to obtain the intermediate compound of Formula W. The intermediate compound of Formula W can undergo aromatic nucleophilic substitution with the carbonyl compound of Formula K to produce the intermediate compound of Formula V. The intermediate compound of Formula V can then cyclize with cyanamide of Formula C to obtain the compound for Formula U.

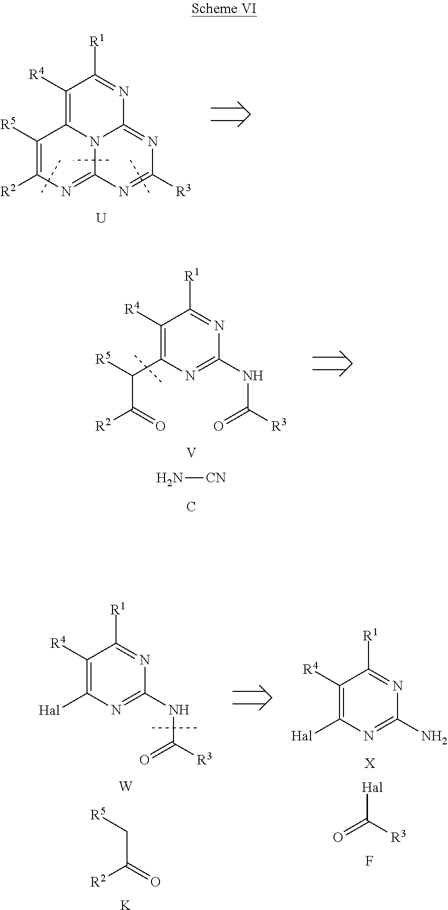

In some embodiments, certain compounds of Formula I (shown as compound of Formula U, wherein X¹ and X² are CR⁴ and CR⁵, respectively, and X³, X⁴, X⁵ and X⁶ are N) are prepared as shown in retrosynthetic Scheme VII. Therefore, the compounds of Formulae N and O can cyclize with the aminopyrimidine compound of Formula Y to produce the compound of Formula U.

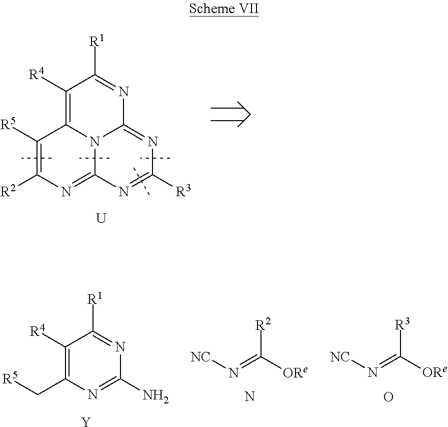

In some embodiments, certain compounds of Formula I (shown as compound of Formula Z, wherein X³ and X⁴ are CR⁶ and CR⁷, respectively, and X¹, X², X⁵ and X⁶ are N) are prepared as shown in retrosynthetic Scheme VIII. Therefore, the enamine compounds of Formulae AC and AD can undergo aromatic nucleophilic substitution with the dihalogenated triazine compound of Formula AB to obtain the intermediate compound of Formula AA, which can then undergo intramolecular cyclization and sequential decarboxylation to generate the compound for Formula Z.

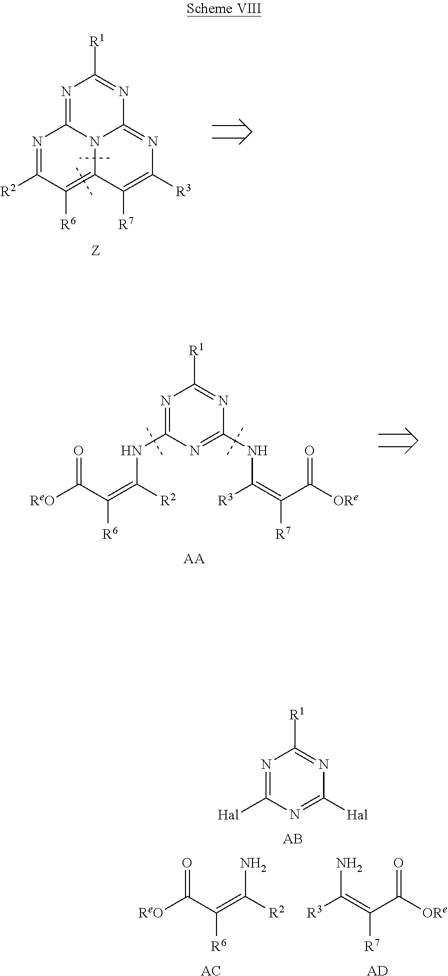

In some embodiments, certain compounds of Formula I (shown as compound of Formula Z, wherein X³ and X⁴ are CR⁶ and CR⁷, respectively, and X¹, X², X⁵ and X⁶ are N) are prepared as shown in retrosynthetic Scheme IX. Therefore, the compound of Formula AF can condense with the diaminotriazine compound of Formula AE to produce the compound of Formula Z.

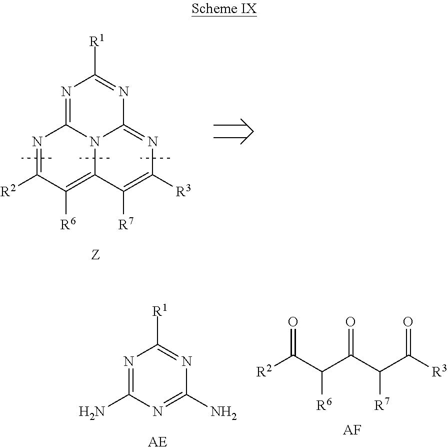

In some embodiments, certain compounds of Formula I-a (shown as compound of Formula AG, wherein X³ and X⁴ are CR⁶ and CR⁷, respectively, R⁶ and R⁷ are linked to form CH═CH and X¹, X², X⁵ and X⁶ are N) are prepared as shown in retrosynthetic Scheme X. Therefore, the cyclopentanone compound of Formula AH can condense with the compound of Formula AE to produce the compound of Formula AG.

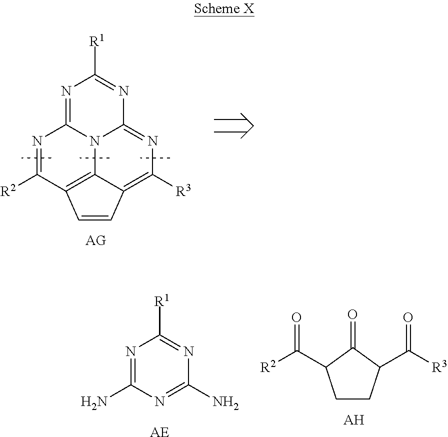

In some embodiments, certain compounds of Formula I-a (shown as compound of Formula AG, wherein X³ and X⁴ are CR⁶ and CR⁷, respectively, R⁶ and R⁷ are linked to form CH═CH and X¹, X², X⁵ and X⁶ are N) are prepared as shown in retrosynthetic Scheme XI. Therefore, the compounds of Formulae AJ and O can cyclize with the bicyclic compound of Formula AI to generate the compound of Formula AG.

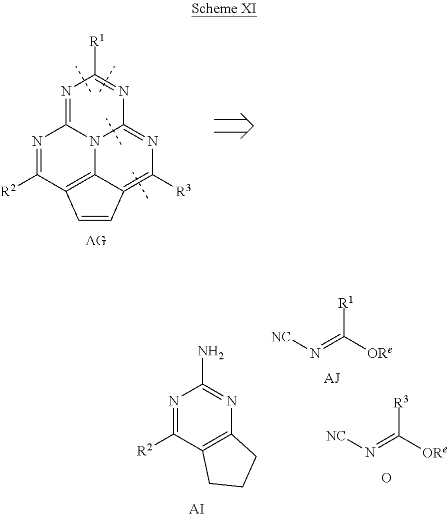

In some embodiments, certain compounds of Formula I-a (shown as compound of Formula AG, wherein X³ and X⁴ are CR⁶ and CR⁷, respectively, R⁶ and R⁷ are linked to form CH═CH and X¹, X², X⁵ and X⁶ are N) are prepared as shown in retrosynthetic Scheme XII. Therefore, the halogenated pyrimidine compound of Formula AN can undergo nucleophilic attack of the hydroxamic acid ester compound of Formula AO to produce the intermediate compound of Formula AL. The intermediate compound of Formula AL can undergo aromatic nucleophilic substitution with the compound of Formula AM to generate the intermediate compound of Formula AK. The intermediate compound of Formula AK can cyclize with cyanamide of Formula C to produce the compound of Formula AG.

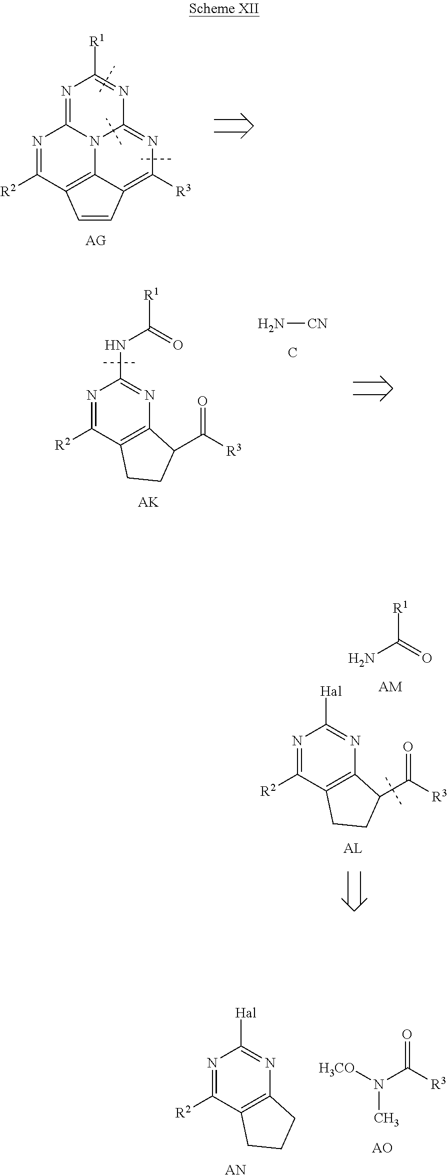

In some embodiments, certain compounds of Formula I-a (shown as compound of Formula AG, wherein X³ and X⁴ are CR⁶ and CR⁷, respectively, R⁶ and R⁷ are linked to form CH═CH and X¹, X², X⁵ and X⁶ are N) are prepared as shown in retrosynthetic Scheme XIII. Therefore, the dicarbonyl compound of Formula AS can cyclise with the tricarbonyl compound of Formula AR to produce the furanone compound of Formula AQ, which can condense with diaminotriazine compound of Formula AE to obtain the intermediate compound of Formula AP. The intermediate compound of Formula AP can undergo alkene metathesis to produce the compound of Formula AG.

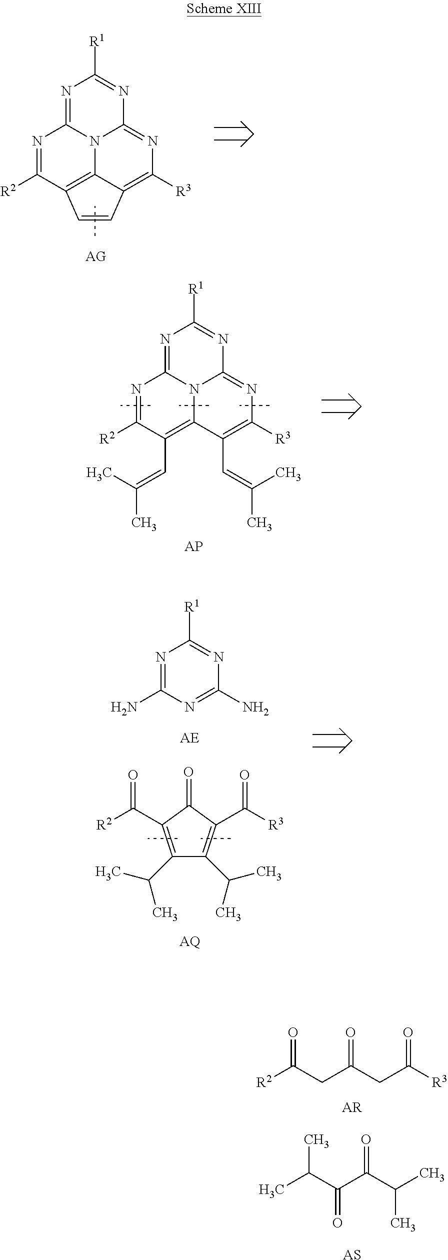

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

### IV. Methods and Uses of the Application

In some embodiments, the present application also includes a use of a compound of the present application in an organic light-emitting diode.

In some embodiments, the compound of the present application is used as an emitter or a dopant.

In some embodiments, the present application also includes a method of preparing an organic light-emitting diode comprising providing at least one compound of the present application as an emitter or a dopant.

In some embodiments, the present application also includes an organic-light emitting diode comprising at least one compound of the present application.

In some embodiments, the present application includes a use of a compound of the present application as a photocatalysis.

In some embodiments, the present application includes a method of performing photocatalysis comprising contacting at least one compound of the present application with a mixture requiring a photocatalyst and performing a photocatalytic transformation on the mixture.

In some embodiments, the present application includes a use of a compound of the present application in the generation of organic laser.

In some embodiments, the present application includes a method of generating organic laser comprising providing at least one compound of the present application as a light emitter.

In some embodiments, the present application also includes an organic-laser comprising at least one compound of the present application.

In some embodiments, the present application includes a use of a compound of the present application in the enhancement of photostability.

In some embodiments, the compound is used as a triplet quencher.

In some embodiments, the present application includes a method of enhancing photostability comprising providing at least one compound of the present application as a triplet quencher.

### EXAMPLES

The following non-limiting examples are illustrative of the present application.

### Example 1 Computation Details

Ground state conformational ensembles were generated using crest (25) (version 2.10.1) with the iMTD-GC (26, 27) workflow (default option) at the GFN0-xTB (28) level of theory. The lowest energy conformers were first reoptimized using xtb (29) (version 6.3.0) at the GFN2-xTB (30, 31) level of theory, followed by another reoptimization using Orca (32, 33) (version 4.2.1) at the B3LYP (34-36)/cc-pVDZ (37) level of theory. The corresponding geometries were used for subsequent ground and excited state single-point calculations. Single points at the ωB2PLYP (38)/def2-SVP (39), and DLPNO-NEVPT2(6,6) (40)/def2-SV(P) (39) levels of theory were performed using Orca (32, 33) (version 4.2.1), single points at the ADC(2) (41-47)/cc-pVDZ (37), ADC(3) (41-47)/cc-pVDZ (37), EOM-CCSD (48-52)/cc-pVDZ (37), FNO-EOM-CCSD (48-56)/cc-pVDZ (37) with 98.85% of the total natural population, and SA-SF-PBE50 (57-62)/def2-SVP (37) levels of theory were performed using Q-Chem (63) (version 5.2). Ground and excited geometry optimizations for adiabatic state energy differences at the ωB2PLYP (38)/def2-SV(P) (39) level of theory were performed using Orca (32, 33) (version 4.2.1). For all excited state single point calculations, four roots were chosen each for both the singlet and the triplet manifold. For the ground and excited state geometry optimizations, two roots were chosen each.

### Gaussian Process Regression

Gaussian process regression was carried out using Python (version 3.6.9) together with the scikit-learn package (version 0.21.2). First, data was transformed linearly to be within the interval [0,1]. As kernel, we used a sum of the Matérn kernel with v=5/2 and the White kernel.

### Example 2 Benchmarking

Methods have been developed to predict the singlet-triplet inversion, which are suitable for high-throughput virtual screening. Several efficient methods were compared against benchmark methods for molecules 1 and 2 (Scheme 1). It was shown previously that single-excitation calculations, including time-dependent density functional approximations (TD-DFA) with GGA, meta-GGA and hybrid functionals, are unable to describe singlet-triplet inversion. (21, 22) Table 1 shows the results of excited state computations for several methods of varying computational cost including two particularly efficient families of methods that include double excitations, namely double-hybrid TD-DFAs (64-67) (ωB2PLYP (38)) and spin-flip TD-DFAs (57, 58) (SA-SF-PBE50 (57-62)). Using ωB2PLYP, vibrational contributions to the singlet-triplet gap were estimated by performing excited singlet and triplet geometry optimizations. Due to their rigid structures, the energy difference between singlet and triplet minima (sometimes termed adiabatic gap) is almost identical to the singlet-triplet gap at the Franck-Condon point (sometimes termed vertical gap) for both 1 and 2. Hence, the latter was used as an approximation to the gap between minima. It was noted that ωB2PLYP only reproduced an inverted singlet-triplet gap for 2, but not for 1. As shown below, this may be the result of a systematic and correctable offset compared to benchmark correlated methods like ADC(2) or EOM-CCS D.

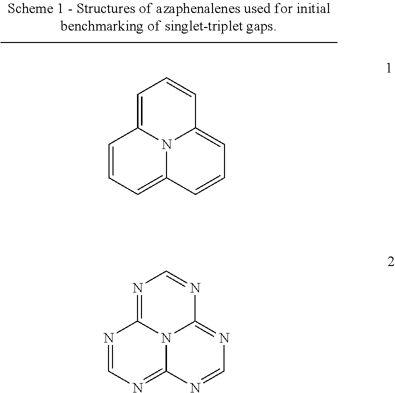 [SMILES: C1=CC2=CC=CC3=CC=CC(=C1)N23 ; C1=NC2=NC=NC3=NC=NC(=N1)N23]

TABLE 1
Benchmarking of excited-state energy differences of 1 and 2. Both double-
hybrid TD-DFAs and spin-flip TD-DFAs can reproduce inverted gaps.

| | 1 | | 2 | |
|---|---|---|---|---|
| | ΔΕ(S₀-S₁) | ΔΕ(S₁-T₁) | ΔΕ(S₀-S₁) | ΔΕ(S₁-T₁) |
| Method | [eV] | [eV] | [eV] | [eV] |
| ADC(3)/cc-pVDZ | 0.777 | −0.092 | 2.665 | −0.109 |
| ADC(2)/cc-pVDZ | 1.038 | −0.160 | 2.578 | −0.278 |
| EOM-CCSD/cc-pVDZ | 1.092 | −0.099 | 2.791 | −0.180 |
| FNO-EOM-CCSD/ | 1.126 | −0.104 | 3.418 | −0.214 |
| cc-pVDZ | | | | |
| DLPNO-NEVPT2(6,6)/ | 1.112 | −0.189 | 2.552 | −0.344 |
| def2-SV(P) | | | | |
| ωB2PLYP/def2-SVP | 1.316 | 0.042 | 3.028 | −0.218 |
| ωB2PLYP/def2-SV(P) | 1.347 | 0.046 | 3.089 | −0.198 |
| (vertical) | | | | |
| ωB2PLYP/def2-SV(P) | 1.296 | 0.055 | 3.045 | −0.188 |
| (adiabatic) | | | | |
| SA-SF-PBE50/def2-SVP | 1.095 | −0.109 | 2.909 | −0.181 |

### Example 3 Effect of Core Structure

Compounds 1 and 2 are isoelectronic and differ only by substitution of C—H with N. Hence, all structures resulting from systematic permutations of such nitrogen substitutions were investigated (Scheme 2).

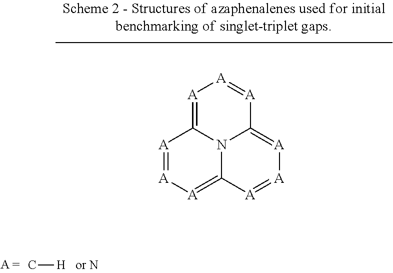

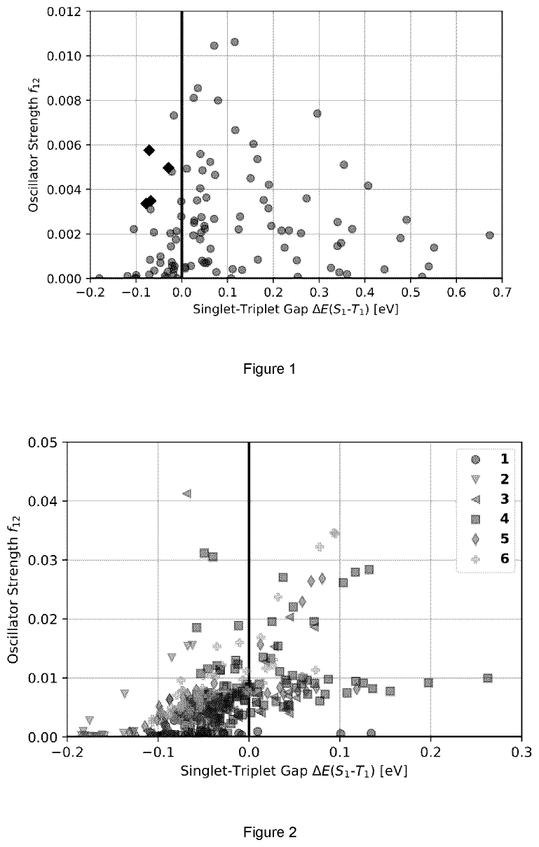

FIG. 1 illustrates the predicted properties of the resulting compounds, at the EOM-CCSD/cc-pVDZ level of theory, with the singlet-triplet gap on the abscissa and the oscillator strength for the S₀-S₁ transition (f₁₂) on the ordinate. It shows that there are several INVEST molecules with non-zero oscillator strength. From these molecules, four have been selected, marked in diamond shapes in FIG. 1 and depicted in Scheme 3, because of their favorable trade-off between the singlet-triplet gap and the oscillator strength, their distinct excitation energies and because synthetic procedures for compounds with these core structures have been reported. (68-84) State energy differences and oscillator strengths of 1-6 are summarized in Table 2.

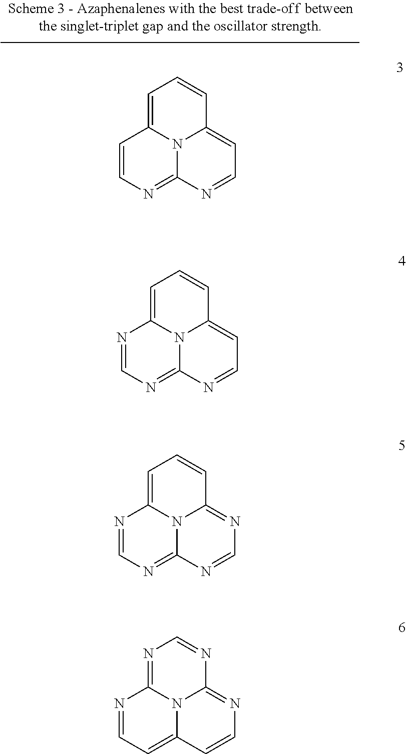 [SMILES: C1=CC2=CC=NC3=NC=CC(=C1)N23 ; C1=CC2=CC=NC3=NC=NC(=C1)N23 ; C1=CC2=NC=NC3=NC=NC(=C1)N23 ; C1=CC2=CC=NC3=NC=NC(=N1)N23]

TABLE 2
Excited-state energy differences and oscillator
strengths of the S0-S1 transition for compounds
1-6 at the EOM-CCSD/cc-pVDZ level of theory.

| EOM-CCSD/ | ΔΕ(S₀-S₁) | ΔΕ(S₁-T₁) | Oscillator |
|---|---|---|---|
| cc-pVDZ | [eV] | [eV] | strength f₁₂ |
| 1 | 1.092 | −0.099 | 0.000 |
| 2 | 2.791 | −0.180 | 0.000 |
| 3 | 1.659 | −0.068 | 0.003 |
| 4 | 2.012 | −0.029 | 0.005 |
| 5 | 2.251 | −0.078 | 0.003 |
| 6 | 2.209 | −0.071 | 0.006 |

### Example 4 Effect of Substitution

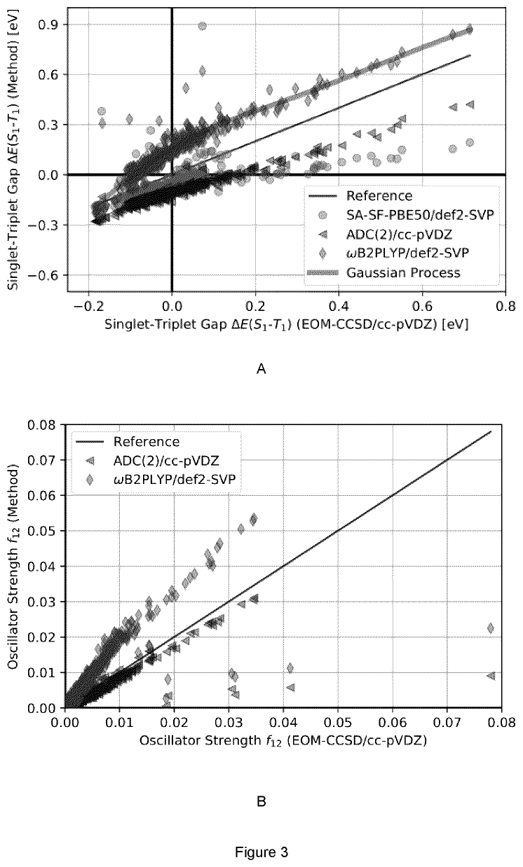

Next, the impact of both electron-donating and electron-withdrawing substituents on the properties was assessed. Both mesomeric and inductive effects were also investigated. Hence, a set of 18 both common and small substituents was selected and the properties for all distinct monosubstituted analogues of compounds 1-6 computed, as depicted in Scheme 4. The corresponding property map, at the EOM-CCSD/cc-pVDZ level of theory, is shown in FIG. 2. In this small set of monosubstituted molecules, there are already a few INVEST molecules with appreciable oscillator strength. These observations suggest that both the singlet-triplet gap and the oscillator strength can be tuned to a significant extent by substituents and that systematic optimization of both these properties is feasible.

Scheme 4-Systematic monosubstitution of compounds
1-6 with diverse substituents.

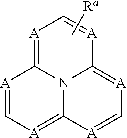

A = C or N
R^{a}

| | | | | | |
|---|---|---|---|---|---|
| —Me | —NH₂ | —OH | —F | —SH | —Cl |
| —Br | —NHMe | —CHCH₂ | —C(O)H | —CCH | —NC |
| —CN | —NMe₂ | —C(O)Me | —S(O)Me | —NO₂ | —CF₃ |

### Example 5 Optimization of Oscillator Strength

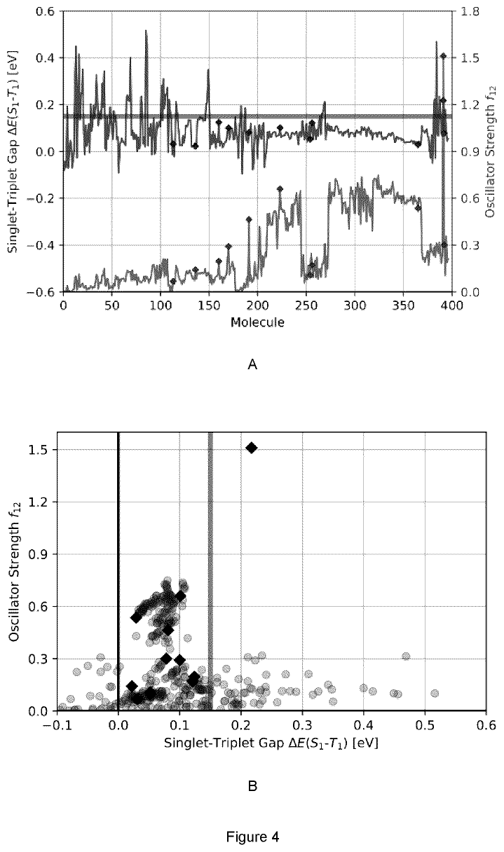

To start optimizing oscillator strength while keeping the singlet-triplet gap negative, a computational protocol was established that predicted trends in the INVEST property, as well as the oscillator strength, and could be efficiently applied to larger molecules. Hence, all EOM-CCSD/cc-pVDZ results, both singlet-triplet gaps and oscillator strengths, of the core structures and monosubstituted compounds were compiled as a benchmark dataset. FIG. 3 compares this dataset against less computationally expensive methods. It shows that ADC(2)/cc-pVDZ generally shows the closest agreement with EOM-CCSD/cc-pVDZ, but at too high a computational cost for screening. ωB2PLYP/def2-SVP offers the suitable trade-off between cost and accuracy, and faithfully reproduces trends in both singlet-triplet gaps and oscillator strengths.

To correct for the systematic offset in the ωB2PLYP/def2-SVP singlet-triplet gaps, Gaussian process regression was performed, and the offset-estimate was determined at an EOM-CCSD/cc-pVDZ singlet-triplet gap of 0 eV. The offset-estimate equals 0.15±0.05 eV. Hence, molecules were optimized by keeping the ωB2PLYP/def2-SVP singlet-triplet gap below 0.15 eV, while maximizing the oscillator strength simultaneously. Without wishing to be bound by theory, outliers in the oscillator strength diagrams (cf. FIG. 3 Panel B) likely stem from EOM-CCSD/cc-pVDZ as a correlation between ADC(2)/cc-pVDZ and ωB2PLYP/def2-SVP oscillator strengths does not show considerable outliers. In addition, to correct for systematic discrepancies in the computed vertical S₁ excitation energies and estimate the solvatochromic shift of the studied compounds in solution, experimental UV-VIS absorption data in solution was compiled from the literature and linear regression used for correction. All predicted absorption wavelengths provided are corrected that way. The underlying data is found in Example 9.

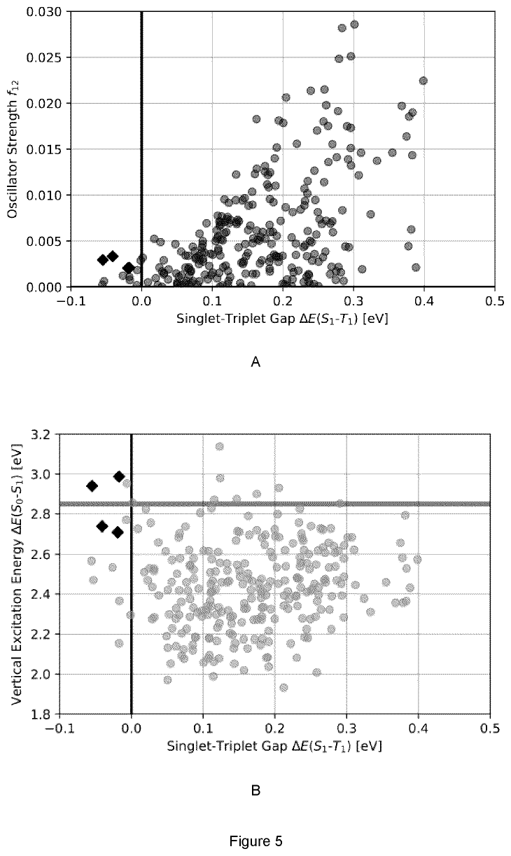

Consequently, INVEST molecules were optimized by systematic structural modification and fine-tuning of properties. The corresponding progress is depicted in FIG. 4. Some notable structures along the trajectory are marked with diamond markers in FIG. 4 Panel A, with diamond-shaped markers in FIG. 4 Panel B and highlighted in Table 3. These results demonstrate that INVEST molecules with appreciable oscillator strength can indeed be designed and are likely not as rare as hypothesized previously. (21)

TABLE 3
Exemplary structures along the optimization trajectory, aimed at INVEST
molecules with appreciable oscillator strength, and their properties.
Absorption wavelengths, A(S₀-S₁), are corrected based on experimental data (vide supra,
details in the Example 9).

| | | ΔE | A | ΔE | |
|---|---|---|---|---|---|
| | | (S₀-S₁) | (S₀-S₁) | (S₁-T₁) | |
| No. | Compound | [eV] | [nm] | [eV] | f₁₂ |
| 7 | 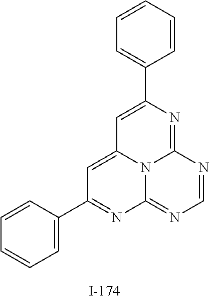 [SMILES: C1=NC2=NC(c3ccccc3)=CC3=CC(c4ccccc4)=NC(=N1)N32] | 2.423 | 594 | 0.031 | 0.067 |
| 8 | 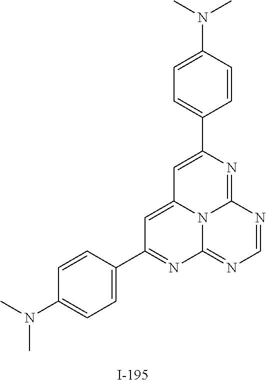 [SMILES: CN(C)c1ccc(C2=CC3=CC(c4ccc(N(C)C)cc4)=NC4=NC=NC(=N2)N34)cc1] | 2.509 | 573 | 0.022 | 0.142 |
| 9 | 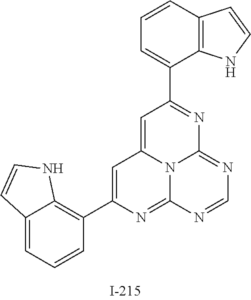 [SMILES: C1=NC2=NC(c3cccc4cc[nH]c34)=CC3=CC(c4cccc5cc[nH]c45)=NC(=N1)N32] | 2.479 | 580 | 0.124 | 0.196 |
| 10 | 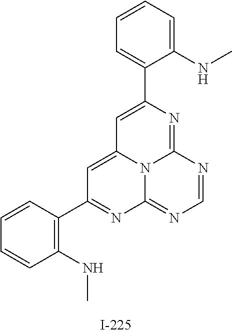 [SMILES: CNc1ccccc1C1=CC2=CC(c3ccccc3NC)=NC3=NC=NC(=N1)N23] | 2.495 | 576 | 0.100 | 0.291 |
| 11 | 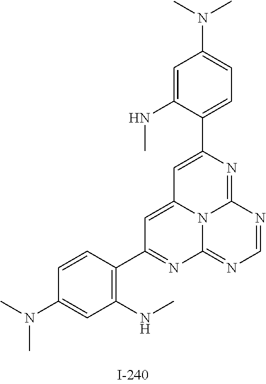 [SMILES: CNc1cc(N(C)C)ccc1C1=CC2=CC(c3ccc(N(C)C)cc3NC)=NC3=NC=NC(=N1)N23] | 2.544 | 563 | 0.081 | 0.464 |
| 12 | 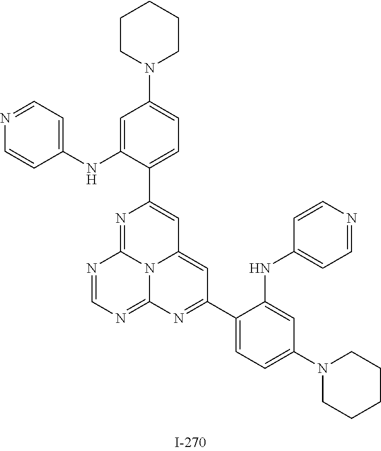 [SMILES: C1=NC2=NC(c3ccc(N4CCCCC4)cc3Nc3ccncc3)=CC3=CC(c4ccc(N5CCCCC5)cc4Nc4ccncc4)=NC(=N1)N32] | 2.533 | 568 | 0.101 | 0.659 |
| 13 | 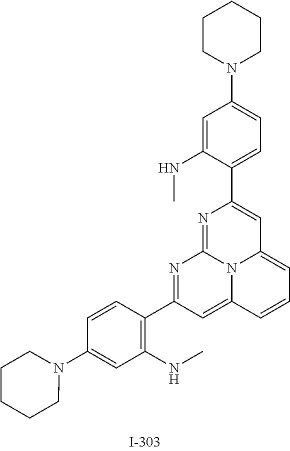 [SMILES: CNc1cc(N2CCCCC2)ccc1C1=CC2=CC=CC3=CC(c4ccc(N5CCCCC5)cc4NC)=NC(=N1)N23] | 2.020 | 714 | 0.052 | 0.106 |
| 14 | 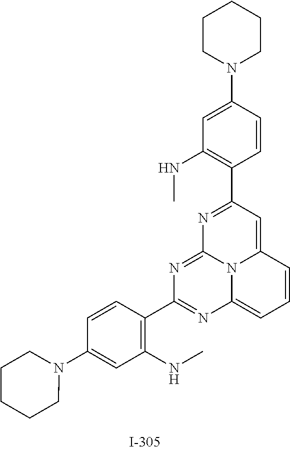 [SMILES: CNc1cc(N2CCCCC2)ccc1C1=NC2=NC(c3ccc(N4CCCCC4)cc3NC)=NC3=CC=CC(=C1)N32] | 2.345 | 614 | 0.121 | 0.171 |
| 15 | 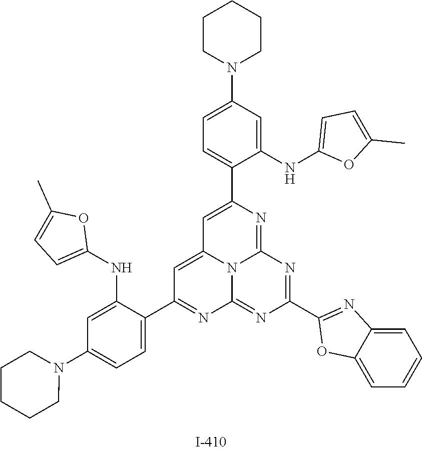 [SMILES: Cc1ccc(Nc2cc(N3CCCCC3)ccc2C2=CC3=CC(c4ccc(N5CCCCC5)cc4Nc4ccc(C)o4)=NC4=NC(c5nc6ccccc6o5)=NC(=N2)N34)o1] | 2.400 | 600 | 0.029 | 0.535 |
| 17 | 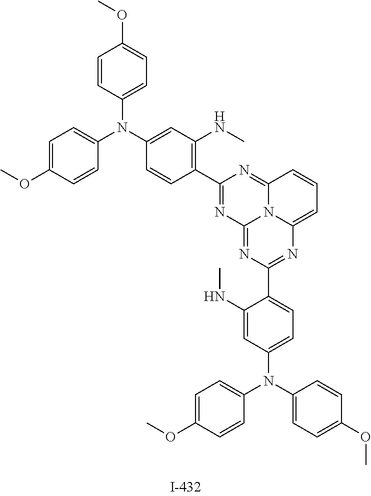 | 2.609 | 551 | 0.078 | 0.300 |

### Example 6 Discovery and Optimization of Blue Emitters

The previous optimization turned out no potential blue INVEST emitters, a color of particular importance in optoelectronic applications (24). Before carrying out a more focused investigation towards INVEST molecules with appreciable oscillator strength, a few modifications of molecules 1 and 2 were tested to find out what structural features revert the inverted singlet-triplet gap. One change that did not revert it, but also increased the vertical excitation energy, is azacyclopenta[cd]phenalene (85) 18, shown in Scheme 5. Hence, analogously to above, all structures resulting from systematic permutations of all possible substitutions of C—H with N were explored (Scheme 5).

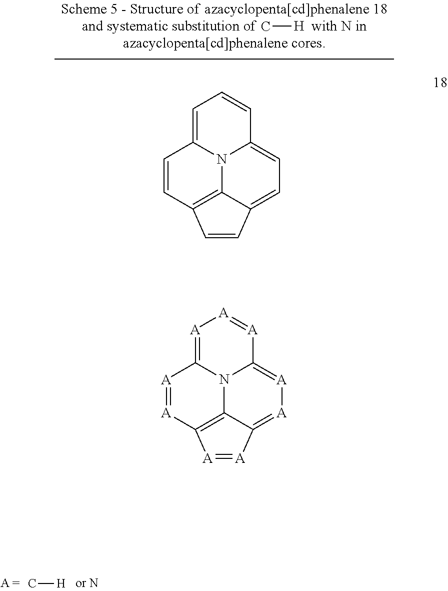

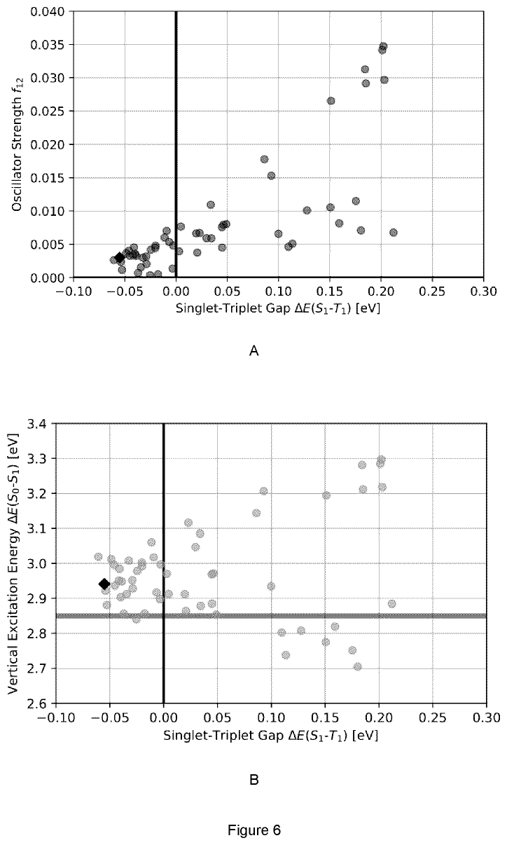

FIG. 5 Panel A shows the map of the singlet-triplet gaps and the oscillator strengths at the EOM-CCSD/cc-pVDZ level of theory and FIG. 5 Panel B shows the map of the singlet-triplet gaps and the vertical excitation energies. Diamond-shaped data points show structures with a good trade-off between the singlet-triplet gap, oscillator strength, and vertical excitation energy. Compared to FIG. 1, the lowest singlet-triplet gaps are larger, the range of singlet-triplet gaps is narrower, and the range of oscillator strengths is wider. At least four exemplary core structures have been identified that showed promising trade-off between singlet-triplet gap, oscillator strength and vertical excitation energy. Their structures are depicted in Scheme 6 and their properties are summarized in Table 4. Compounds 20-22 are derivatives of 4 and 6, some of the most promising INVEST core structures identified in the previous sections, thus it was not very surprising these structures would be among the ones with the best combination of properties for blue INVEST emitters. Notably, none of the four azacyclopenta[cd]phenalenes 19-22 have been reported in the literature before, and only derivatives of 18 have been synthesized previously. (86-88)

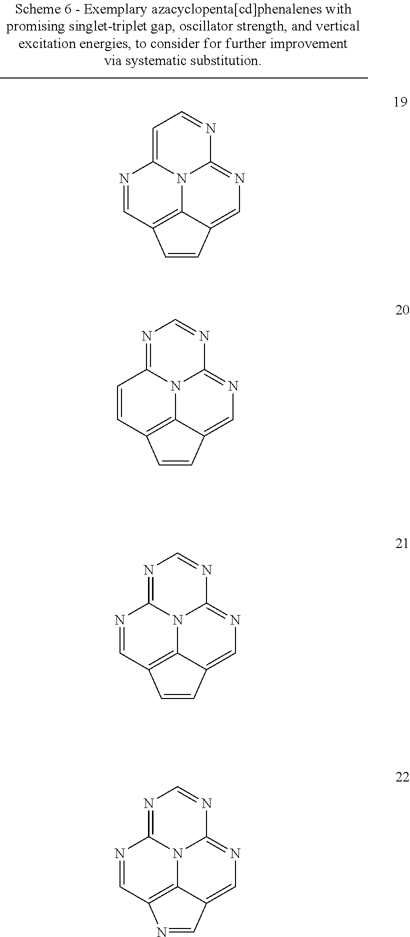 [SMILES: c1cc2ncc3ccc4cnc(n1)n2c34 ; c1nc2ccc3ccc4cnc(n1)n2c34 ; c1nc2ncc3ccc4cnc(n1)n2c34 ; c1nc2ncc3cnc4cnc(n1)n2c34]

TABLE 4
Excited state energy differences and oscillator
strengths of the S₀-S₁ transition
for compounds 18-22 at the
EOM-CCSD/cc-pVDZ level of theory

| | ΔΕ(S₀-S₁) | λ(S₀-S₁) | ΔΕ(S₁-T₁) | |
|---|---|---|---|---|
| Compound | [eV] | [nm] | [eV] | f₁₂ |
| 18 | 2.153 | 607 | −0.017 | 0.001 |
| 19 | 2.738 | 486 | −0.041 | 0.003 |
| 20 | 2.708 | 491 | −0.019 | 0.002 |
| 21 | 2.941 | 455 | −0.055 | 0.003 |
| 22 | 2.987 | 448 | −0.017 | 0.002 |

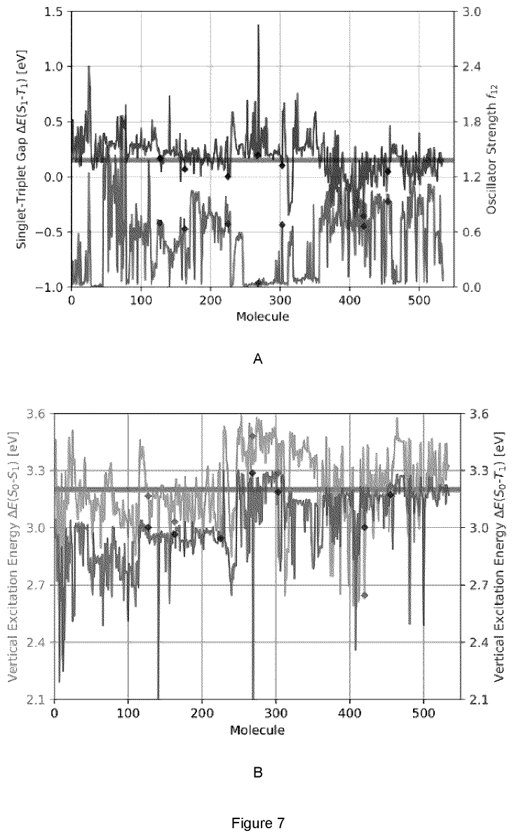

Consequently, compound 21 was used as a basis for further substitution optimization because it offers the best trade-off of all these four structures and studied all distinct monosubstituted analogues with the same set of 18 substituents used with the azaphenalenes, as depicted in Scheme 7. The corresponding property maps at the EOM-CCSD/cc-pVDZ level of theory are shown in FIG. 6. The results show that tuning of the singlet-triplet gap, oscillator strength and vertical excitation energy can be achieved to a significant extent even with a single substitution.

Scheme 7-Systematic monosubstitution of
compounds 21 with diverse substituents.

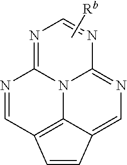

R^{b}

| | | | | | |
|---|---|---|---|---|---|
| —Me | —NH₂ | —OH | —F | —SH | —Cl |
| —Br | —NHMe | —CHCH₂ | —C(O)H | —CCH | —NC |
| —CN | —NMe₂ | —C(O)Me | —S(O)Me | —NO₂ | —CF₃ |

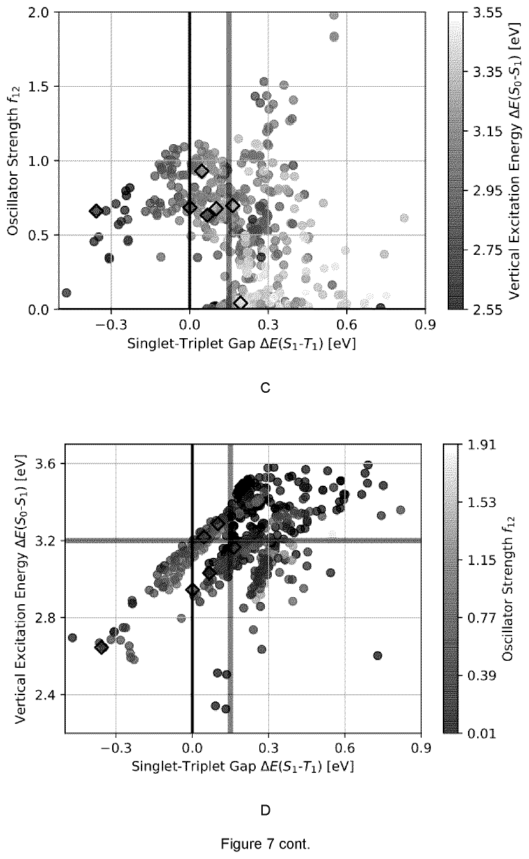

Having identified compound 21 as the most promising azacyclopenta[cd]phenalene core structure and studied the effect of small substituents on its properties, systematic optimization was done to find substituted analogues of 21 with inverted singlet-triplet gaps, appreciable oscillator strength and vertical excitation energies suitable for blue emitters. Hence, this time three target properties were to be optimized simultaneously. The optimization progress is illustrated in FIG. 7. Again, important structures along the optimization trajectory are marked with diamond markers in FIG. 7a-b, with red markers in FIG. 7c-d, and highlighted in Table 5. These results show that blue INVEST emitters can very likely be realized, and they demonstrate again that INVEST molecules with appreciable oscillator strength are likely more common than expected previously.

TABLE 5
Important structures along the optimization trajectory, aimed at potential blue
INVEST emitters, and their properties.
Absorption wavelengths, λ(S₀-S₁), are corrected based on experimental data (vide supra,
details in the Example 9).

| | | ΔE | A | ΔE | |
|---|---|---|---|---|---|
| | | (S₀-S₁) | (S₀-S₁) | (S₁-T₁) | |
| No. | Compound | [eV] | [nm] | [eV] | f₁₂ |
| 24 | 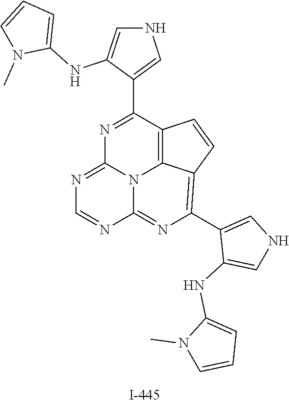 [SMILES: Cn1cccc1Nc1c[nH]cc1-c1nc2ncnc3nc(-c4c[nH]cc4Nc4cccn4C)c4ccc1c4n23] | 3.031 | 473 | 0.067 | 0.633 |
| 25 | 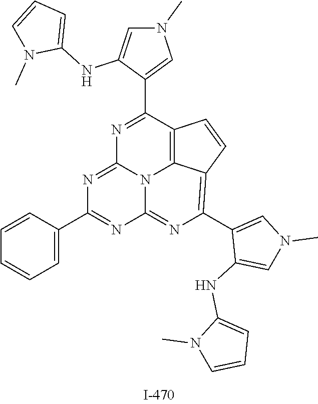 [SMILES: Cn1cc(Nc2cccn2C)c(-c2nc3nc(-c4ccccc4)nc4nc(-c5cn(C)cc5Nc5cccn5C)c5ccc2c5n34)c1] | 2.944 | 488 | 0.001 | 0.684 |
| 27 | 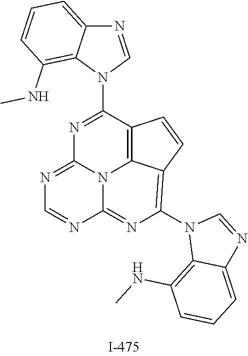 [SMILES: CNc1cccc2ncn(-c3nc4ncnc5nc(-n6cnc7cccc(NC)c76)c6ccc3c6n45)c12] | 3.287 | 436 | 0.101 | 0.677 |
| 28 | 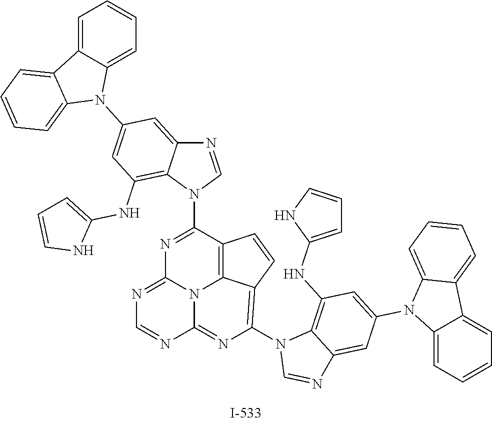 [SMILES: c1c[nH]c(Nc2cc(-n3c4ccccc4c4ccccc43)cc3ncn(-c4nc5ncnc6nc(-n7cnc8cc(-n9c%10ccccc%10c%10ccccc%109)cc(Nc9ccc[nH]9)c87)c7ccc4c7n56)c23)c1] | 2.645 | 543 | −0.357 | 0.661 |
| 29 | 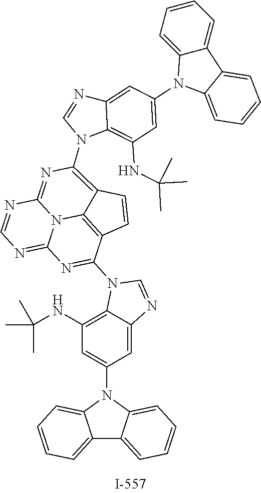 [SMILES: CC(C)(C)Nc1cc(-n2c3ccccc3c3ccccc32)cc2ncn(-c3nc4ncnc5nc(-n6cnc7cc(-n8c9ccccc9c9ccccc98)cc(NC(C)(C)C)c76)c6ccc3c6n45)c12] | 3.218 | 446 | 0.046 | 0.929 |

### Example 7 Validation of Optimized Structures

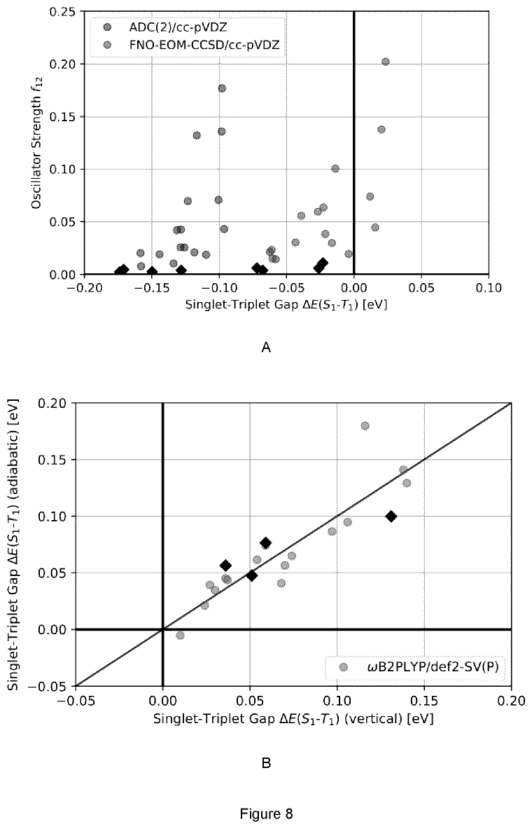

To validate the structures generated, minimal analogues of promising structures identified above were used to confirm their properties using higher-level theory. Furthermore, vibrational contributions to the singlet-triplet gaps were evaluated as above and tested for the possibility of excited-state intramolecular proton transfer (ESIPT) (89-97) in hydrogen-bonded INVEST molecules. The minimal analogues selected are defined in Scheme 8. The results of high-level theory methods, as well as the comparison between Franck-Condon (vertical) and minima-to-minima (adiabatic) singlet-triplet gaps, are illustrated in FIG. 8. The benchmark methods depicted in FIG. 8 Panel A confirm the significant increase in oscillator strength obtained while (largely) maintaining the inverted gaps, as observed at the ωB2PLYP/def2-SVP level of theory. Notably, the minimal analogues selected for validation are neither the best candidates found in terms of inverted singlet-triplet gaps nor in terms of oscillator strength yet they still show promise for use as INVEST emitters in applications. Furthermore, FIG. 8 Panel B shows that vibrational contributions to the singlet-triplet gap are generally negligible for the minimal analogues selected. The largest adverse vibrational effect was observed for compound 41, but it still amounts only to 0.06 eV.

Scheme 8-Minimal analogues of INVEST molecules with
appreciable oscillator strength used for validation.

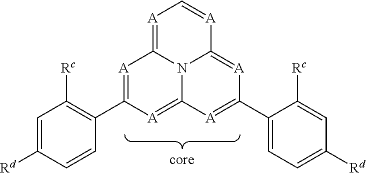

A = C—H or N

| Compound | Core | R^{c} | R^{d} |
|---|---|---|---|
| 30 | 3 | H | H |
| 31 | 3 | NH₂ | H |
| 32 | 3 | H | NH₂ |
| 33 | 3 | NH₂ | NH₂ |
| 34 | 4 | H | H |
| 35 | 4 | NH₂ | H |
| 36 | 4 | H | NH₂ |
| 37 | 4 | NH₂ | NH₂ |
| 38 | 5 | H | H |
| 39 | 5 | NH₂ | H |
| 40 | 5 | H | NH₂ |
| 41 | 5 | NH₂ | NH₂ |
| 23 | 6 | H | H |
| 42 | 6 | NH₂ | H |
| 43 | 6 | H | NH₂ |
| 44 | 6 | NH₂ | NH₂ |

Finally, the possibility of ESIPT was tested in all validation compounds with intramolecular hydrogen bonds, namely 31, 33, 35, 37, 39, 41, 42 and 44. Both single and double proton transfer from the aniline to the respective hydrogen-bonded core nitrogen atom were tested by displacing the hydrogen atom accordingly and optimizing the resulting structures in the S₀, S₁ and T₁ manifolds, respectively. The corresponding results are provided in Table 6. For almost all compounds, neither single (1 PT), nor double (2 PT) proton transfer results in a stable state in the S₁ manifold as geometry optimization reversed the proton transfer(s) back to the original structures. In the S₀ manifold, proton transfer never resulted in a stable state. In the T₁ manifold, single proton transfer generally resulted in stable states, which were energetically uphill for all validation compounds except 42. Nevertheless, for 42, single proton transfer was energetically downhill only by about 0.08 eV. Double proton transfer resulted in a stable state in the T₁ manifold only for 44. Hence, ESIPT is unlikely to cause significant property changes to the INVEST molecules studied herein.

TABLE 6
Test for excited-state intramolecular proton transfer
(ESIPT) in minimal analogues of INVEST molecules
with appreciable oscillator strength.

| | E(S₀) [eV] | | E(S₁) [eV] | | E(T₁) [eV] | |
|---|---|---|---|---|---|---|
| Compound | 1 PT | 2 PT | 1 PT | 2 PT | 1 PT | 2 PT |
| 31 | — | — | — | — | +0.49 | — |
| 33 | — | — | — | — | +0.62 | — |
| 35 | — | — | — | — | +0.34 | — |
| 37 | — | — | — | — | +0.56 | — |
| 39 | — | — | +0.86 | — | +0.05 | — |
| 41 | — | — | — | — | +0.31 | — |
| 42 | — | — | — | — | −0.08 | — |
| 44 | — | — | — | — | +0.11 | +0.98 |

The table entries provide the energy differences of the proton transfer states (PT) to the corresponding initial states in the respective state manifolds (S₀, S₁ or T₁) at the ωB2PLYP/def2-SV(P) level of theory. Unstable structures, denoted as “-,” showed reverse proton transfer during geometry optimization.

### Example 8 Discussion and Conclusion

It has been shown that modification of phenalene cores results in a rich chemical space of INVEST molecules as the singlet-triplet gap, oscillator strength and absorption wavelength can be tuned over wide property intervals.

Further, it has been shown that INVEST molecules with appreciable oscillator strength are possible, and can be realized by careful modification of substituents on azaphenalenes.

Moreover, it has been shown that INVEST molecules with appreciable oscillator strength based on azaphenalenes cores cover substantially the entire visible light spectrum and thus can be used as organic electronic materials for various applications, especially OLED materials.

In the present application, organic molecules with inverted singlet-triplet gaps based on nitrogen-substituted phenalenes have been explored computationally. Through substitution of azaphenalenes with a combination of π-substituents, donor, and acceptor groups, a number of INVEST molecules with appreciable oscillator strength was revealed. In addition, by modifying the phenalene core, and investigating azacyclopenta[cd]phenalenes, blue INVEST emitters with considerable oscillator strength were identified. These molecules are synthetically accessible and offer various advantages for optoelectronic applications, including potentially fast reverse intersystem crossing, increased device lifetime and high color purity.

### Example 9 Solvatochromic Shift Calibration

Table 7 provides the data used for calibrating for the solvatochromic shift with the corresponding references. Table 8 provides the results of linear regressions carried out for that purpose. These linear regressions were used to estimate the absorption wavelength for the compounds investigated in the course of this study.

TABLE 7
Calibration of solvatochromic shift using experimental absorption data.

| | | EOM-CCSD/ | AE(S0-S1) [eV] | ωB2PLYP/ | SA-SF-PBE50/ |
|---|---|---|---|---|---|
| Compound | Experiment | cc-pVDZ | ADC(2)/cc-pVDZ | def2-SVP | def2-SVP |
| 1 | 1.039 (98) | 1.092 | 1.038 | 1.316 | 1.095 |
| | (hexane) | | | | |
| 3 | 1.908 (99) | 1.659 | 1.536 | 1.881 | 1.635 |
| | (EtOH) | | | | |
| 4 | 1.845 (100) | 2.012 | 1.863 | 2.226 | 1.957 |
| | (EtOH) | | | | |
| 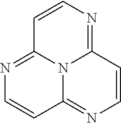 [SMILES: C1=CC2=NC=CC3=NC=CC(=N1)N23] | 1.974 (101) (hexane) | 2.264 | 2.062 | 2.421 | 2.163 |
| S46 | | | | | |
| 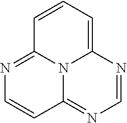 [SMILES: C1=CC2=NC=CC3=NC=NC(=C1)N23] | 1.962 (102) (EtOH) | 1.999 | 1.852 | 2.213 | 1.988 |
| S47 | | | | | |
| 5 | 2.039 (103) | 2.251 | 2.093 | 2.479 | 2.210 |
| | (MeCN) | | | | |
| 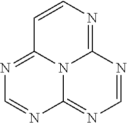 [SMILES: C1=CC2=NC=NC3=NC=NC(=N1)N23] | 2.335 (103) (MeCN) | 2.526 | 2.333 | 2.755 | 2.518 |
| S53 | | | | | |
| 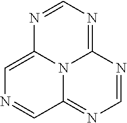 [SMILES: C1=NC=C2N=CN=C3N=CN=C1N23] | 1.947 (103) (MeCN) | 2.114 | 1.970 | 2.333 | 2.016 |
| S77 | | | | | |
| 2 | 2.799 (104) | 2.791 | 2.578 | 3.028 | 2.909 |
| | (MeCN) | | | | |
| 7 | 1.950 (105) | 2.197 | 1.963 | 2.423 | 2.193 |
| (I-174) | (CHCl3) | | | | |
| 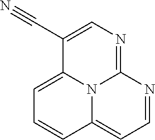 [SMILES: N#CC1=CN=C2N=CC=C3C=CC=C1N32] | 1.807 (99) (EtOH) | 1.786 | 1.651 | 2.011 | 1.764 |
| S210 | | | | | |

The solvents used in experiment, if known, are added in parenthesis. Computations were carried out without solvent model.

TABLE 8
Results of linear regression of experimental against predicted vertical S₂
excitation energies: ΔE(S₀ − S₁)ₑₓₚ = Slope · ΔE(S₀ − S₁)_{com} + Intercept

| Method | Slope | Intercept [eV] | R² | F | N |
|---|---|---|---|---|---|
| EOM-CCSD/cc-pVDZ | 0.87(11) | 0.17(22) | 0.88 | 67 | 11 |
| ADC(2)/cc-pVDZ | 0.96(11) | 0.13(22) | 0.89 | 72 | 11 |
| ωB2PLYP/def2-SVP | 0.87(10) | −0.03(23) | 0.89 | 74 | 11 |
| SA-SF-PBE50/def2-SVP | 0.85(9) | 0.23(18) | 0.91 | 93 | 11 |

### Example 10

The above computational results were confirmed using a more robust method as described below.

Error! Reference source not found.9 shows the results of several computational excited state techniques of varying computational cost including two particularly efficient families of methods that include double excitations, namely double-hybrid TD-DFAs (ωB2PLYP′¹¹⁰) and spin-flip TD-DFAs^{111,112} (SA-SF-PBE50¹¹¹⁻¹¹⁶). As no currently available program can compute the perturbative doubles correction for the excited triplet energies of range-separated double-hybrid functionals such as ωB2PLYP,¹¹⁷ the singlet-triplet gap was computed by subtracting the first excited triplet energy without the doubles correction from the first excited singlet energy, which includes the doubles correction. In this study, this method is denoted by ωB2PLYP′. It is noted that ωB2PLYP′ only reproduces an inverted singlet-triplet gap for 2, but not for 1. Without wishing to be bound theory, this is the result of a systematic and correctable offset compared to benchmark methods like ADC(2) or EOM-CCSD (vide infra).

TABLE 9
Benchmarking of excited-state energy differences of 1 and 2. Both double-
hybrid TD-DFAs and spin-flip TD-DFAs can reproduce inverted gaps.

| | 1 | | 2 | |
|---|---|---|---|---|
| | ΔΕ(S₀ − S₁) | ΔΕ(S₁ − S₁) | ΔΕ(S₀ − S₁) | ΔΕ(S₁ − S₁) |
| Method | [eV] | [eV] | [eV] | [eV] |
| ADC(3)/cc-pVDZ | 0.777 | −0.092 | 2.665 | −0.109 |
| ADC(2)/cc-pVDZ | 1.038 | −0.160 | 2.578 | −0.278 |
| ADC(2)/cc-pVDZ/IEFPCM(S₀) | 1.029 | −0.161 | 2.657 | −0.281 |
| ADC(2)/aug-cc-pVDZ | 1.006 | −0.144 | 2.614 | −0.263 |
| EOM-CCSD/cc-pVDZ | 1.092 | −0.099 | 2.791 | −0.180 |
| FNO-EOM-CCSD/cc-pVDZ | 1.126 | −0.104 | 3.418 | −0.214 |
| FNO-EOM-CCSD/aug-cc- | 1.178 | −0.086 | 3.040 | −0.167 |
| pVDZ | | | | |
| DLPNO-NEVPT2(6,6)/def2- | 1.112 | −0.189 | 2.552 | −0.344 |
| SV(P) | | | | |
| ωB2PLYP′/def2-SVP | 1.316 | 0.042 | 3.028 | −0.218 |
| ωB2PLYP′/def2-SVP/C-PCM | 1.303 | 0.036 | 3.165 | −0.236 |
| ωB2PLYP′/def2-SV(P) | 1.347 | 0.046 | 3.089 | −0.198 |
| (vertical) | | | | |
| ωB2PLYP′/def2-SV(P) | 1.296 | 0.055 | 3.045 | −0.188 |
| (adiabatic) | | | | |
| SA-SF-PBE50/def2-SVP | 1.095 | −0.109 | 2.909 | −0.181 |

To obtain an estimate of the impact of omitting the doubles correction for the excited triplets, RI-CIS(D)/def2-SVP calculations were performed for the benchmark dataset. The results show that the doubles correction, in principle, can be both stabilizing and destabilizing for the first excited triplet, but tends to be stabilizing with a median of about −0.1 eV. For the first excited singlet, the doubles correction is always strongly stabilizing, and its median is about ten times as large. This suggests that the impact of omitting the doubles correction for the excited triplets is likely not large.

Finally, extensive simulations were performed evaluating the properties of 1 in amorphous solid-state thin films using a mixed QM/MM approach. Table provides the average and standard deviations of oscillator strength and singlet-triplet gap, respectively, of conformers of 1 extracted from the thin film simulations carried out, both the results with and without accounting for the point charge clouds approximating the environment within the thin films. The results show that the effect of the environment in thin films does not affect the inverted singlet-triplet gaps.

TABLE 10
Averages and standard deviations of properties of conformers of 1
extracted from the amorphous solid-state thin film simulations.
Results are at the ωB2PLYP′/def2-SVP level of theory.

| | Singlet-Triplet Gap [eV] | | Oscillator Strength | |
|---|---|---|---|---|
| Thin Film | Point Charges | Vacuum | Point Charges | Vacuum |
| Pure 1 | 0.046 ± 0.000 | 0.043 ± 0.000 | 0.0004 ± 0.0000 | 0.0000 ± 0.0000 |
| 1 in mCP | 0.045 ± 0.001 | 0.043 ± 0.000 | 0.0007 ± 0.0001 | 0.0000 ± 0.0000 |
| 1 in | 0.043 ± 0.002 | 0.043 ± 0.000 | 0.0014 ± 0.0003 | 0.0000 ± 0.0000 |
| DPEPO | | | | |

“Point Charges” denotes the corresponding calculations including the point charges approximating the solid-state environment.
“Vacuum” denotes the results of the same conformers but without accounting for the solid-state environment via point charges.

It was found that in none of the thin-films simulated the spectroscopic properties of 1 changed significantly, both singlet-triplet gaps and oscillator strengths were largely unaffected. This suggests that the inverted singlet-triplet gaps are at least not intrinsically affected by the solid-state environment.

Comparison of Vertical and Adiabatic Singlet-Triplet Gaps. The comparison of vertical and adiabatic gaps from ωB2PLYP′ calculations was also investigated for the benchmark set. The corresponding results are illustrated in Error! Reference source not found.9. It shows that the deviation between adiabatic and vertical singlet-triplet gaps generally is larger in magnitude the larger the singlet-triplet gap. Hence, for molecules with inverted singlet-triplet gaps, the corresponding corrections tend to be very small. However, there are a few outliers with significantly more positive adiabatic singlet-triplet gaps, which all correspond to monosubstituted derivatives of 2 with oxygen-containing functional groups (one ketone, one aldehyde and one nitro group). Notably, there are also compounds for which the corresponding corrections can lead to significantly smaller singlet-triplet gaps. Importantly, the associated deviation tends to be negligible for INVEST molecules and over the entire benchmark set the average difference between adiabatic and vertical singlet-triplet gaps only surmounts to 0.02 eV. This shows that the vertical singlet-triplet gaps are generally a good approximation of the adiabatic singlet-triplet gaps in the INVEST emitters studied in this work.

For further validation, RI-ADC(2)/cc-pVDZ calculations were performed for compounds 8-15 and 17. The corresponding results are provided in Table 11. They show that all the compounds are predicted to have inverted singlet-triplet gaps confirming our ωB2PLYP′/def2-SVP results and showing that the systematic offset seen in the benchmark data is valid for larger compounds as well. In addition, the observed trends in the oscillator strengths at the ωB2PLYP′/def2-SVP level of theory were well reproduced with RI-ADC(2)/cc-pVDZ.

TABLE 11
RI-ADC(2)/cc-pVDZ results for structures along the optimization trajectory,
aimed at INVEST molecules with appreciable oscillator strength.

| | | ΔE | ΔE | |
|---|---|---|---|---|
| | | (S₀-S₁) | (S₁-T₁) | |
| No. | Compound | [eV] | [eV] | f₁₂ |
| 7 | 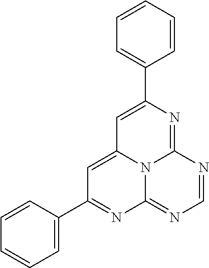 [SMILES: C1=NC2=NC(c3ccccc3)=CC3=CC(c4ccccc4)=NC(=N1)N32] | 1.962 | −0.128 | 0.031 |
| | I-174 | | | |
| 8 | 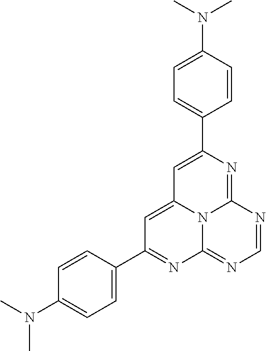 [SMILES: CN(C)c1ccc(C2=CC3=CC(c4ccc(N(C)C)cc4)=NC4=NC=NC(=N2)N34)cc1] | 2.043 | −0.131 | 0.072 |
| | I-195 | | | |
| 9 | 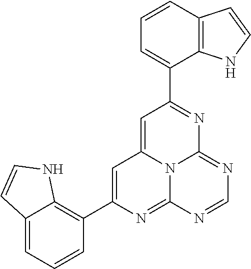 [SMILES: C1=NC2=NC(c3cccc4cc[nH]c34)=CC3=CC(c4cccc5cc[nH]c45)=NC(=N1)N32] | 2.008 | −0.083 | 0.096 |
| | I-215 | | | |
| 10 | 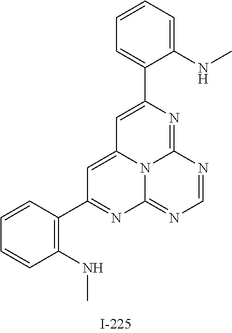 [SMILES: CNc1ccccc1C1=CC2=CC(c3ccccc3NC)=NC3=NC=NC(=N1)N23] | 2.006 | −0.067 | 0.175 |
| 11 | 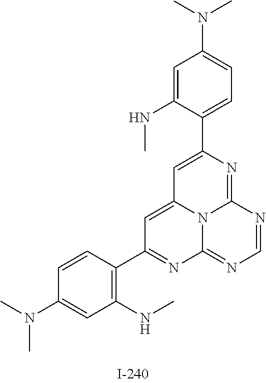 [SMILES: CNc1cc(N(C)C)ccc1C1=CC2=CC(c3ccc(N(C)C)cc3NC)=NC3=NC=NC(=N1)N23] | 2.057 | −0.069 | 0.304 |
| 12 | 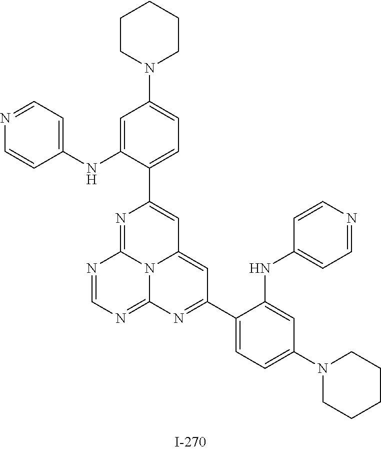 [SMILES: C1=NC2=NC(c3ccc(N4CCCCC4)cc3Nc3ccncc3)=CC3=CC(c4ccc(N5CCCCC5)cc4Nc4ccncc4)=NC(=N1)N32] | 2.020 | −0.053 | 0.475 |
| 13 | 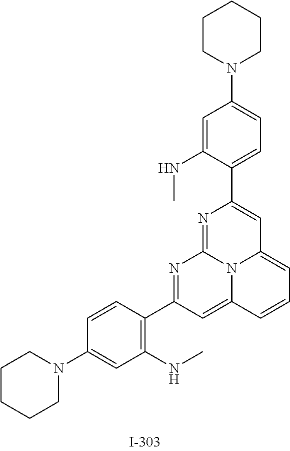 [SMILES: CNc1cc(N2CCCCC2)ccc1C1=CC2=CC=CC3=CC(c4ccc(N5CCCCC5)cc4NC)=NC(=N1)N23] | 1.581 | −0.126 | 0.054 |
| 14 | 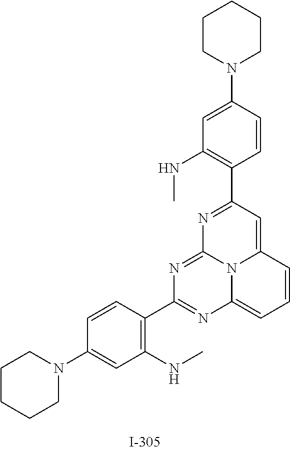 [SMILES: CNc1cc(N2CCCCC2)ccc1C1=NC2=NC(c3ccc(N4CCCCC4)cc3NC)=NC3=CC=CC(=C1)N32] | 1.871 | −0.093 | 0.094 |
| 15 | 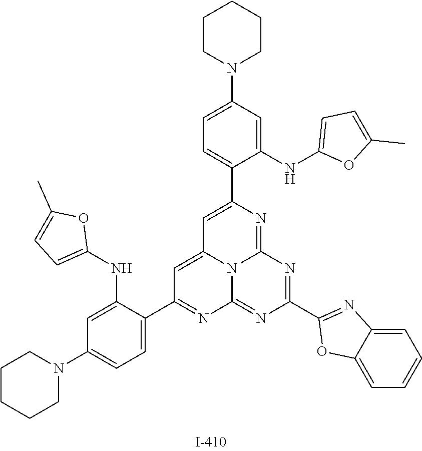 [SMILES: Cc1ccc(Nc2cc(N3CCCCC3)ccc2C2=CC3=CC(c4ccc(N5CCCCC5)cc4Nc4ccc(C)o4)=NC4=NC(c5nc6ccccc6o5)=NC(=N2)N34)o1] | 1.820 | −0.036 | 0.346 |
| 17 | 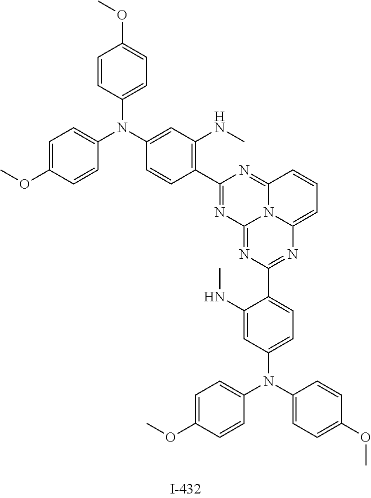 | 2.083 | −0.096 | 0.204 |

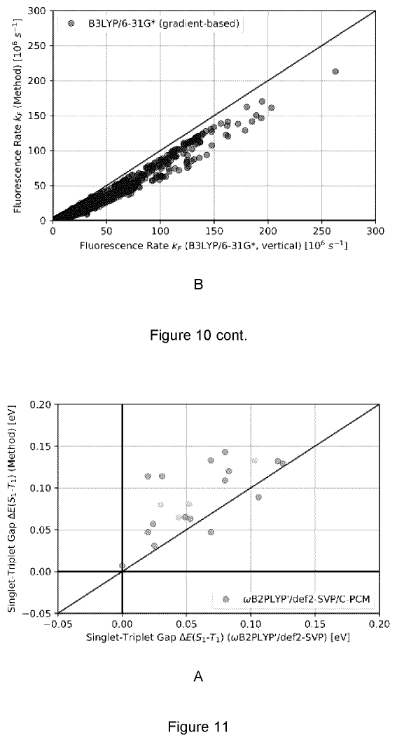

Finally, the impact of excited state relaxation on both emission energies was evaluated and compared to vertical transition energies, and fluorescence rates. To do this, absorption and emission spectra including Franck-Condon factors were computed using a path integral approach¹¹⁸⁻¹¹⁹ at the B3LYP/6-31G* level of theory (FIG. 10). Error! Reference source not found.10A shows that the difference between the vertical excitation energies and the emission energies are for almost all compounds small as the corresponding difference amounts to less than 0.30 eV for more than 80% of the compounds. The emission energies calculated this way were, on average, 0.22 eV below the corresponding vertical transition energies, with a standard deviation of 0.17 eV. Moreover, Error! Reference source not found.10B shows that the fluorescence rate estimates obtained from absorption oscillator strength show excellent agreement with estimates obtained from the more sophisticated Franck-Condon calculations. This suggests that the absorption wavelengths can be used to approximate the emission wavelength, with the proviso that it will be an upper bound. Furthermore, these results also show that estimating fluorescence rates from absorption oscillator strengths and vertical excitation energies is a good approximation.

Influence of the Environment in an Emitter. Moreover, the influence of the environment in an emitter at the ωB2PLYP′/def2-SVP/C-PCM level of theory was also investigated on the same compound series (Table 12).

TABLE 12
Minimal analogues of INVEST molecules with appreciable
fluorescence rates used for validation.

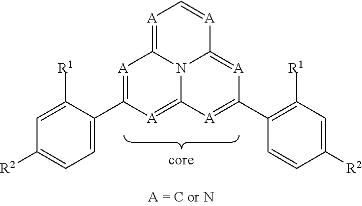

| Compound | Core | R¹ | R² |
|---|---|---|---|
| 30 | 3 | H | H |
| 31 | 3 | NH₂ | H |
| 32 | 3 | H | NH₂ |
| 33 | 3 | NH₂ | NH₂ |
| 34 | 4 | H | H |
| 35 | 4 | NH₂ | H |
| 36 | 4 | H | NH₂ |
| 37 | 4 | NH₂ | NH₂ |
| 38 | 5 | H | H |
| 39 | 5 | NH₂ | H |
| 40 | 5 | H | NH₂ |
| 41 | 5 | NH₂ | NH₂ |
| 23 | 6 | H | H |
| 42 | 6 | NH₂ | H |
| 43 | 6 | H | NH₂ |
| 44 | 6 | NH₂ | NH₂ |

The corresponding influence was evaluated for the molecules used for benchmarking. Solvent environment effects on the minimal analogues of the structures described herein were also assessed. The corresponding results are depicted in Error! Reference source not found.11. It shows that the influence of the solid-state solvation is very small with the largest adverse correction only surmounting to 0.09 eV and on average only to 0.03 eV. Interestingly, as illustrated in Error! Reference source not found.11B, the oscillator strength tends to be increased by the solid-state solvation by about 18%. Hence, the small adverse effects observed for the singlet-triplet gaps are compensated for by higher oscillator strength values facilitating emission.

### Computational Methods

Ground state conformational ensembles were generated using crest¹²⁰ (version 2.10.1) with the iMTD-GC¹²¹⁻¹²² workflow (default option) at the GFN0-xTB¹²³ level of theory. The lowest energy conformers were first reoptimized using xtb¹²⁴ (version 6.3.0) at the GFN2-xTB¹²⁵⁻¹²⁶ level of theory, followed by another reoptimization using Orca¹²⁷⁻¹²⁸ (version 4.2.1) at the B3LYP¹²⁹⁻¹³¹/cc-pVDZ¹³² level of theory. The corresponding geometries were used for subsequent ground and excited state single-point calculations. Single points at the ωB2PLYP′¹¹⁰/def2-SVP,¹³³ and DLPNO-NEVPT2(6,6)¹³⁴/def2-SV(P)¹³³ levels of theory were performed using Orca¹²⁸⁻¹²⁸ (version 4.2.1), single points at the RI-ADC(2)¹³⁵⁻¹⁴¹/cc-pVDZ,¹³² RI-ADC(2)¹³⁵⁻¹⁴¹/aug-cc-pVDZ,^{132, 142} RI-ADC(3)¹³⁵⁻¹⁴¹/cc-pVDZ,¹³² RI¹⁴³⁻¹⁴⁵-CIS(D)¹⁴⁶⁻¹⁴⁷/def2-SVP, RI-EOM-CCSD¹⁴⁸⁻¹⁵²/cc-pVDZ,¹³² RI-FNO-EOM-CCSD¹⁴⁸⁻¹⁵⁶/cc-pVDZ¹³² and RI-FNO-EOM-CCSD¹⁴³⁻¹⁵¹/aug-cc-pVDZ^{132,142} with 98.85% of the total natural population, and SA-SF-PBE50¹¹¹⁻¹¹⁶/def2-SVP¹³² levels of theory were performed using Q-Chem¹⁵⁷ (version 5.2). RI-ADC(2)¹³⁵⁻¹⁴¹/cc-pVDZ¹³² calculations for large molecules (8-15 and 17) were performed using TURBOMOLE^{158, 159} (version 7.4.1). Ground and excited geometry optimizations for adiabatic state energy differences at the ωB2PLYP′¹¹⁰/def2-SV(P)¹³³ level of theory were performed in Orca¹²⁷⁻¹²⁸ (version 4.2.1) using numerical gradients. Single point calculations with implicit solvent corrections at the ωB2PLYP′¹¹⁰/def2-SVP¹³³/C-PCM¹⁶⁰ level of theory were performed using Orca¹²⁷⁻¹²⁸ (version 4.2.1) and at the ADC(2)¹³⁵⁻¹⁴¹/cc-pVDZ¹³²/IEFPCM¹⁶¹⁻¹⁶² level of theory using Q-Chem¹⁵⁷ (version 5.2) assuming a dielectric constant of 4.0¹⁶³⁻¹⁶⁴ and a refractive index of 1.8.¹⁶⁵⁻¹⁶⁷ Importantly, in the Orca version used (version 4.2.1), the perturbative doubles correction is not applied to the excited triplet states.¹¹⁷ Hence, to indicate this explicitly, the corresponding method was termed ωB2PLYP′ as opposed to ωB2PLYP. For all excited state single point calculations, four roots were chosen each for both the singlet and the triplet manifold. For the ground and excited state geometry optimizations, two roots were chosen each. Fluorescence rate estimates provided in the tables in the main text are based on absorption oscillator strengths and vertical excitation energies, which are used first to compute transition dipole moments, and converted to fluorescence rates based on well-established equations from the literature.¹¹⁹ These values are intended to convey an idea as to the order of magnitude of the emission rate¹⁶⁸ and to help compare the brightness of INVEST emitters with, for example, those of well-known emitters.

More sophisticated emission wavelength and fluorescence rate calculations were performed using Franck-Condon calculations via a gradient-based method, which was described previously,¹¹⁸⁻¹¹⁹ at the previously benchmarked¹⁶⁸⁻¹⁶⁹ B3LYP¹²⁹⁻¹³¹/6-31G*¹⁷⁰⁻¹⁷² level of theory using Q-Chem¹⁵⁷ (version 5.3). For each molecule, a geometry optimization was performed to obtain the minimum energy geometry of the electronic ground state R₀ and the Hessian matrix H₀(R₀) was calculated. Excited-state minimum energy geometries Rᵢ were estimated using energy gradients gᵢ(R₀) computed with TD-DFT,⁶⁸ Rᵢ=R₀+[H₀(R₀)]⁻¹gᵢ(R₀). Vibronic time-dependent correlation functions were evaluated using the displaced harmonic oscillator equations.¹⁷⁴ The correlation functions were multiplied by a broadening factor, Γ(t)=e^{-σ}^{²}^{t}^{²}^{/2-|γ|t}. The Fourier transform of those functions yields the Franck-Condon factors, from which the extinction function, the fluorescence rate and emission power spectral density can be recovered.¹¹⁹ The broadening factor corresponds to a Voigt profile in the frequency domain and the values of α and γ were chosen to obtain inhomogeneous and homogeneous widths of 200 cm⁻¹ and 5 cm⁻¹, respectively. Emission was taken to occur solely through the S₁→S₀ transition, in accordance with Kasha's rule.¹⁷⁵

To evaluate the effect of solid state embedding on the inverted singlet-triplet gap, a multiscale simulation protocol based on molecular dynamics was used for the generation of amorphous thin film morphologies and a quantum mechanical embedding scheme that self-consistently evaluates the partial charges of each (polarized) molecule in the thin film. The point charge clouds were used as an embedding to compute the excited S₁ and T₁ states. In detail, atomistically resolved amorphous thin films were generated using the Metropolis Monte Carlo based vapor deposition simulation protocol Deposit,¹⁷⁶ based on a DFT parameterized dihedral force field, using B3LYP¹²⁹⁻¹³¹/def2-SV(P)¹³³ as reference. For mixed guest-host systems, 2000 1,3-bis(N-carbazolyl)benzene (mCP) or bis[2-(diphenylphosphino)phenyl]ether oxide (DPEPO) host molecules and 200 molecules of 1 were used. For each molecule in the system, partial charges were computed using the self-consistent embedding protocol Quantum Patch, at the B3LYP¹²⁹⁻¹³¹/def2-SV(P)¹³³ level of theory.¹⁷⁷ These partial charges were then used in ωB2PLYP′¹¹⁰/def2-SVP¹³³ computations to emulate a polarized solid-state environment at the QM level.

### Example 11 Preparation of Compound I-428

Exemplary compound I-428 was prepared as described below.

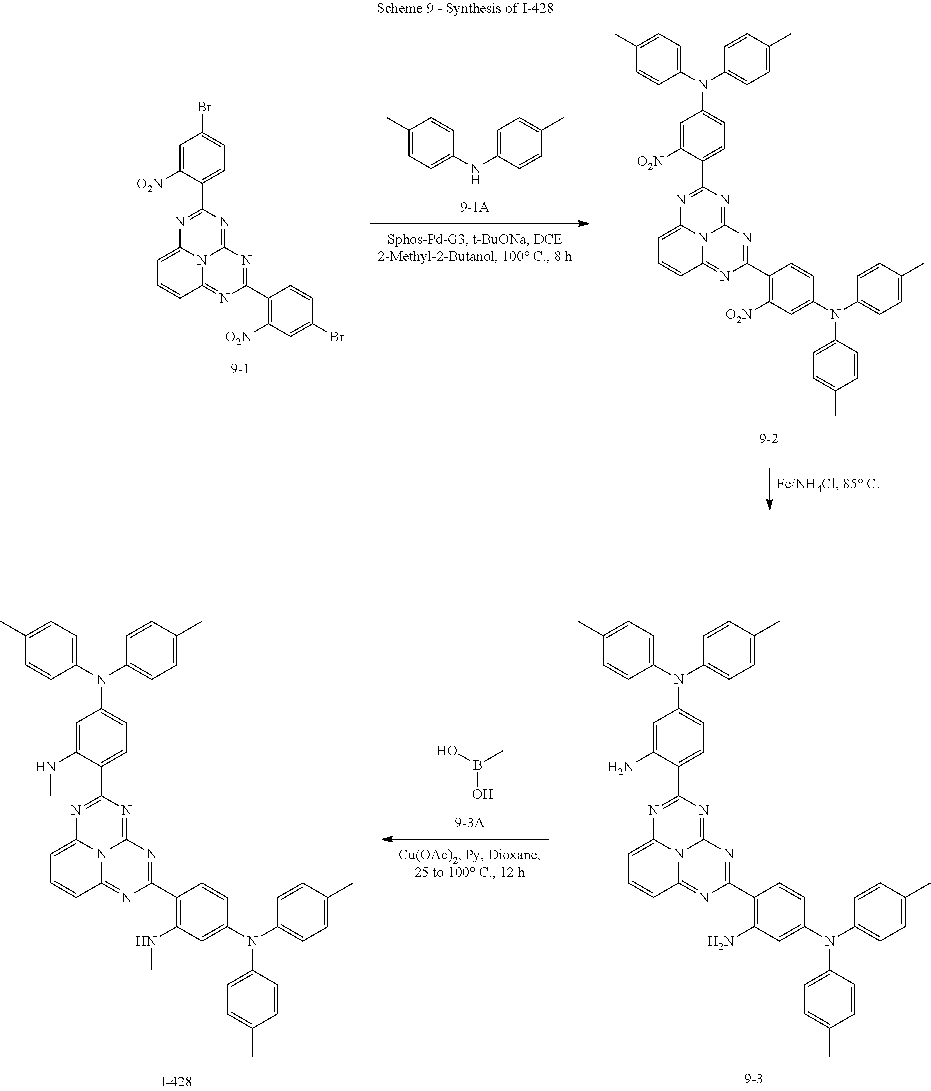

### Compound 9-2

A mixture of Compound 9-1 (1.00 g, 1.75 mmol), Compound 9-1A (622 mg, 3.15 mmol), SPhos-Pd-G3 (273 mg, 0.35 mmol) and t-BuONa (337 mg, 3.50 mmol) in 2-methylbutan-2-ol (15 mL) was degassed and purged with N₂ for 3 times, and then the mixture was stirred at 100° C. for 8 h under N₂ atmosphere. The reaction mixture was concentrated under reduced pressure to give a residue. The residue was purified by column chromatography (SiO2, Petroleum ether/Ethyl acetate=30/1 to 5/1) to give Compound 9-2 (0.40 g, 0.50 mmol, 28% yield) was obtained as a black-brown solid.

### Compound 9-3

To a mixture of Compound 9-2 (56 mg, 0.07 mmol) in EtOH (3 mL) and H₂O (1 mL) was added Fe (16 mg, 0.28 mmol) and NH₄Cl (15 mg, 28 mmol). The mixture was stirred at 85° C. for 1 h. The organic volatiles were removed under reduced pressure to give a residue. The residue was purified by Prep-TLC (DCM) to give Compound 9-3 (20 mg, 0.03 mmol, 39% yield) as a gray solid.

¹H NMR (EC1230-58-P1) (400 MHz, DMSO-d6) δ 7.84 (d, J=9.2 Hz, 2H), 7.41 (t, J=8.4 Hz, 1H), 7.35-7.09 (m, 12H), 7.00 (d, J=8.0 Hz, 8H), 6.18 (d, J=8.4 Hz, 2H), 6.07-5.94 (m, 4H), 2.29 (s, 12H)

### Compound I-428

To a solution of Compound 9-3 (200 mg, 0.27 mmol) and Py (149 mg, 1.88 mmol, 0.2 mL) in dioxane (6 mL) was added Cu(OAc)₂ (166 mg, 0.91 mmol) and stirred at 25° C. for 0.25 h, then Compound 9-3A (48 mg, 0.81 mmol) was added to the mixture and stirred at 100° C. for 11.75 h. The organic volatiles were remove under reduced pressure to give a residue. The residue was purified by prep-TLC (SiO₂, DCM) to give Compound I-428 (45 mg, 0.05 mmol, 20% yield, 94% purity) as a brown solid.

LCMS: EC1230-113-P1B, t_{R}=0.794 min, MS (ESI+) m/z=772.4[M+1].

HPLC: EC1230-112-P1D, t_{R}=2.727 min, Purity=94.86%.

¹H NMR (EC1230-113-P1D) (400 MHz, DMSO-d₆) δ 8.97-8.90 (m, 2H), 7.98 (d, J=9.2 Hz, 2H), 7.47-7.43 (m, 1H), 7.21-7.14 (m, 8H), 7.06 (d, J=8.4 Hz, 8H), 6.24 (d, J=8.0 Hz, 2H), 6.03 (dd, J=2.2, 8.8 Hz, 2H), 5.96 (d, J=2.0 Hz, 2H), 2.61 (s, 6H), 2.32 (s, 12H)

### Example 11 Preparation of Compound I-432

Exemplary Compound I-432 was prepared as described below.

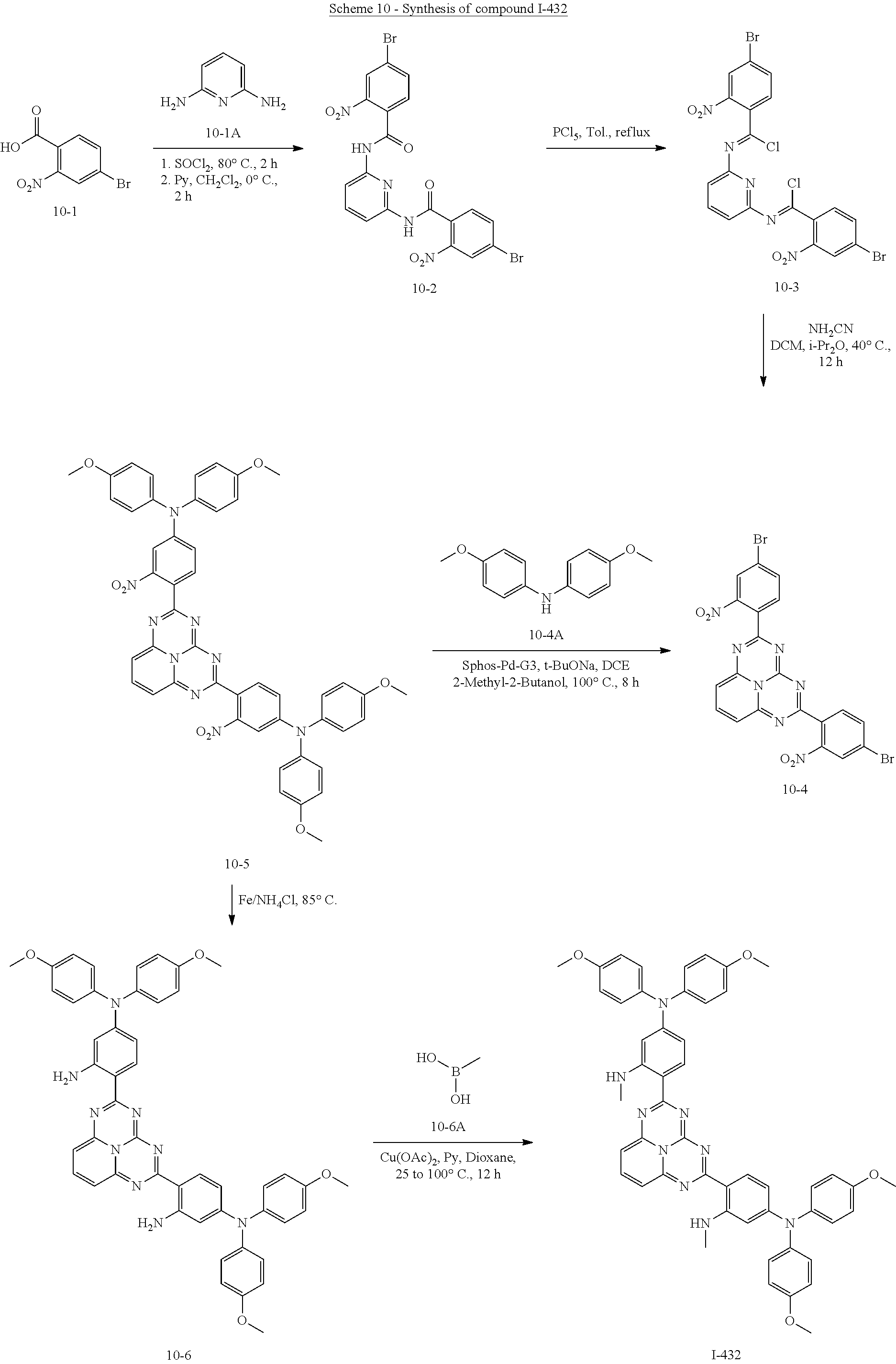

### Compound 10-2

A mixture of Compound 10-1 (2.00 g, 8.13 mmol) in SOCl₂ (10 mL) was degassed and purged with N₂ for 3 times and the mixture was stirred at 80° C. for 2 h under N₂ atmosphere. TLC (PE/EA=4/1) showed Compound 10-1 was consumed completely. The reaction mixture was concentrated under reduced pressure to give a residue, which was used directly. To a solution of Compound 10-1A (0.43 g, 3.97 mmol) in DCM (10 mL) was added Pyridine (0.94 g, 11.91 mmol) at 0° C. Then the former residue in DCM (5 mL) was slowly added to the reaction mixture and the mixture was stirred at 0° C. for 2 h. The reaction mixture was concentrated under reduced pressure to give a residue. The residue was purified by prep-HPLC (column: 330 g Flash Coulmn Welch Ultimate XB_C18 20-40 μm; mobile phase: [water-ACN]; B %: 5-40% 30 min; 40% 5 min) to give Compound 10-2 (0.40 g, 70.71 mmol, 18% yield) as a brown solid.

¹H NMR (EC1230-41-P1) (400 MHz, DMSO-d₆) δ 11.00 (s, 2H), 8.36 (d, J=2.0 Hz, 2H), 8.08 (dd, J=2.0, 8.0 Hz, 2H), 7.97-7.80 (m, 3H), 7.71 (d, J=8.0 Hz, 2H)

### Compound 10-3

A mixture of Compound 10-2 (350 mg, 0.92 mmol), PCl₅ (388 mg, 1.86 mmol) in toluene (3 mL) was degassed and purged with N₂ for 3 times, and then the mixture was stirred at 120° C. for 3 h under N₂ atmosphere. TLC (PE/EA=4/1) showed Compound 10-2 was consumed and one main spot formed. The reaction mixture was concentrated under reduced pressure to give Compound 10-3 (390 mg, crude) as a brown oil, which was used into the next step without further purification.

### Compound 10-4

To a solution of Compound 10-3 (1.80 g, 2.99 mmol) in DCM (20 mL) was added NH₂CN (1.51 g, 35.88 mmol) in i-Pr₂O (10 mL), then the reaction mixture was stirred at 40° C. for 12 h. The reaction mixture was concentrated under reduced pressure to give a residue. The residue was triturated with MeOH (40 mL) for 30 min to give Compound 10-4 (1.15 g, 2.01 mmol, 67% yield) as a green solid.

¹H NMR (EC1230-58-P1) (400 MHz, DMSO-d₆) δ 8.21 (d, J=2.0 Hz, 2H), 7.99 (dd, J=2.0, 8.4 Hz, 2H), 7.86 (d, J=8.4 Hz, 2H), 7.54 (t, J=8.4 Hz, 1H), 6.23 (d, J=8.4 Hz, 2H)

### Compound 10-5

A mixture of Compound 10-4 (1.00 g, 1.75 mmol), Compound 10-4A (0.72 g, 3.15 mmol), Sphos-Pd-G3 (0.27 g, 0.35 mmol) and t-BuONa (0.34 g, 3.50 mmol) in 2-methylbutan-2-ol (15 mL) was degassed and purged with N₂ for 3 times and then the mixture was stirred at 100° C. for 8 h under N₂ atmosphere. The mixture was concentrated under reduced pressure to give a residue. The residue was purified by column chromatography (SiO₂, DCM) to give Compound 10-5 (0.40 g, 0.46 mmol, 26% yield) as a brown solid.

### Compound 10-6

A mixture of Compound 10-5 (200 mg, 0.23 mmol), Fe (128 mg, 2.30 mmol) and NH₄Cl (123 mg, 2.30 mmol) in dioxane (12 mL) and H₂O (4 mL) was heated to 85° C. and the mixture was stired at 85° C. for 1 h. The reaction mixture was concentrated under reduced pressure to give a residue. The residue was purified by Prep-TLC (SiO₂, DCM) to give Compound 10-6 (40 mg, 0.05 mmol, 21% yield) as a red solid.

### Compound I-432

To a solution of Compound 6 (150 mg, 0.19 mmol) and Py (103 mg, 1.30 mmol, 0.1 mL) in dioxane (6 mL) was added Cu(OAc)₂ (115 mg, 0.63 mmol). The mixture was stirred at 25° C. for 0.25 h, then Compound 6A (33 mg, 0.56 mmol) was added to the mixture and the mixture was stirred at 100° C. for 11.75 h. The reaction mixture was concentrated under reduced pressure to give a residue. The residue was purified by prep-TLC (SiO₂, DCM) to give Compound UT20201112B (57 mg, 0.06 mmol, 35% yield, 95% purity) as a brown solid.

LCMS: EC1230-112-P1E, t_{R}=0.700 min, MS (ESI+) m/z=836.3[M+1].

HPLC: EC1230-112-P1F, t_{R}=3.248 min, Purity=95.37%.

¹H NMR (EC1230-112-P1A) (400 MHz, DMSO-d₆) δ 9.10-8.96 (m, 2H), 7.93 (d, J=9.2 Hz, 2H), 7.44 (t, J=8.4 Hz, 1H), 7.20-7.09 (m, 8H), 7.00-6.92 (m, 8H), 6.23 (d, J=8.4 Hz, 2H), 5.91 (dd, J=2.4, 9.2 Hz, 2H), 5.77-5.75 (m, 2H), 3.76 (s, 12H), 2.56 (d, J=4.8 Hz, 6H)

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATION OF DOCUMENTS CITED IN THE PRESENT APPLICATION

(1) Hund, F. Zur Deutung verwickelter Spektren, insbesondere der Elemente Scandium bis Nickel. Z. Physik 1925, 33, 345-371.
(2) Koseki, S.; Nakajima, T.; Toyota, A. Violation of Hund's Multiplicity Rule in the Electronically Excited States of Conjugated Hydrocarbons. Can. J. Chem. 1985, 63, 1572-1579.
(3) Kutzelnigg, W.; Morgan, J. D. Hund's Rules. Z Phys D—Atoms, Molecules and Clusters 1996, 36, 197-214.
(4) Jablonski, A. Efficiency of Anti-Stokes Fluorescence in Dyes. Nature 1933, 131, 839-840.
(5) Valeur, B.; Berberan-Santos, M. N. A Brief History of Fluorescence and Phosphorescence before the Emergence of Quantum Theory. J. Chem. Educ. 2011, 88, 731-738.
(6) Farr, E. P.; Quintana, J. C.; Reynoso, V.; Ruberry, J. D.; Shin, W. R.; Swartz, K. R. Introduction to Time-Resolved Spectroscopy: Nanosecond Transient Absorption and Time-Resolved Fluorescence of Eosin B. J. Chem. Educ. 2018, 95, 864-871.
(7) Leermakers, P. A.; Vesley, G. F. Organic Photochemistry and the Excited State. J. Chem. Educ. 1964, 41, 535.
(8) Swenton, J. S. Photochemistry of Organic Compounds. I, Selected Aspects of Olefin Photochemistry. J. Chem. Educ. 1969, 46, 7.
(9) Miller, J. B. Photodynamic Therapy: The Sensitization of Cancer Cells to Light. J. Chem. Educ. 1999, 76, 592.
(10) Demas, J. N. Photophysical Pathways in Metal Complexes. J. Chem. Educ. 1983, 60, 803.
(11) Richards, J. H. Physical-Organic Chemistry. J. Chem. Educ. 1968, 45, 398.
(12) Jaffe, H. H.; Miller, A. L. The Fates of Electronic Excitation Energy. J. Chem. Educ. 1966, 43, 469.
(13) Olivier, Y.; Sancho-Garcia, J.-C.; Muccioli, L.; D'Avino, G.; Beljonne, D. Computational Design of Thermally Activated Delayed Fluorescence Materials: The Challenges Ahead. J. Phys. Chem. Lett. 2018, 9, 6149-6163.
(14) Difley, S.; Beljonne, D.; Van Voorhis, T. On the Singlet-Triplet Splitting of Geminate Electron-Hole Pairs in Organic Semiconductors. J. Am. Chem. Soc. 2008, 130, 3420-3427.
(15) Eizner, E.; Martinez-Martinez, L. A.; Yuen-Zhou, J.; Kéna-Cohen, S. Inverting Singlet and Triplet Excited States Using Strong Light-Matter Coupling. Science Advances 2019, 5, eaax4482.
(16) Olivier, Y.; Yurash, B.; Muccioli, L.; D'Avino, G.; Mikhnenko, O.; Sancho-García, J. C.; Adachi, C.; Nguyen, T.-Q.; Beljonne, D. Nature of the Singlet and Triplet Excitations Mediating Thermally Activated Delayed Fluorescence. Phys. Rev. Materials 2017, 1, 075602.
(17) Toyota, A.; Nakajima, T. Violation of Hund's Multiplicity Rule in the Lowest Excited Singlet-Triplet Pairs of Cyclic Bicalicene and Its Higher Homologues. Journal of the Chemical Society, Perkin Transactions 2 1986, 0, 1731-1734.
(18) Toyota, A. Violation of Hund's Rule in the Lowest Excited Singlet-Triplet Pairs of Dicyclohepta[Cd,Gh]Pentalene and Dicyclopenta[Ef,KI]Heptalene. Theoret. Chim. Acta 1988, 74, 209-217.
(19) Leupin, W.; Wirz, J. Low-Lying Electronically Excited States of Cycl[3.3.3]Azine, a Bridged 12.Pi.-Perimeter. J. Am. Chem. Soc. 1980, 102, 6068-6075.
(20) Leupin, Werner.; Magde, Douglas.; Persy, Gabriele.; Wirz, Jakob. 1,4,7-Triazacycl[3.3.3]Azine: Basicity, Photoelectron Spectrum, Photophysical Properties. J. Am. Chem. Soc. 1986, 108, 17-22.
(21) de Silva, P. Inverted Singlet-Triplet Gaps and Their Relevance to Thermally Activated Delayed Fluorescence. J. Phys. Chem. Lett. 2019, 10, 5674-5679.
(22) Ehrmaier, J.; Rabe, E. J.; Pristash, S. R.; Corp, K. L.; Schlenker, C. W.; Sobolewski, A. L.; Domcke, W. Singlet-Triplet Inversion in Heptazine and in Polymeric Carbon Nitrides. J. Phys. Chem. A 2019, 123, 8099-8108.
(23) Reid, D. H. The Chemistry of the Phenalenes. Q. Rev. Chem. Soc. 1965, 19, 274-302.
(24) Wong, M. Y.; Zysman-Colman, E. Purely Organic Thermally Activated Delayed Fluorescence Materials for Organic Light-Emitting Diodes. Advanced Materials 2017, 29, 1605444.
(25) grimme-lab/crest https://github.com/grimme-lab/crest (accessed Jun. 3, 2020).
(26) Grimme, S. Exploration of Chemical Compound, Conformer, and Reaction Space with Meta-Dynamics Simulations Based on Tight-Binding Quantum Chemical Calculations. J. Chem. Theory Comput. 2019, 15, 2847-2862.
(27) Pracht, P.; Bohle, F.; Grimme, S. Automated Exploration of the Low-Energy Chemical Space with Fast Quantum Chemical Methods. Phys. Chem. Chem. Phys. 2020, 22, 7169-7192.
(28) Pracht, P.; Caldeweyher, E.; Ehlert, S.; Grimme, S. A Robust Non-Self-Consistent Tight-Binding Quantum Chemistry Method for Large Molecules. ChemRxiv 2019.
(29) grimme-lab/xtb https://github.com/grimme-lab/xtb (accessed Jun. 3, 2020).
(30) Grimme, S.; Bannwarth, C.; Shushkov, P. A Robust and Accurate Tight-Binding Quantum Chemical Method for Structures, Vibrational Frequencies, and Noncovalent Interactions of Large Molecular Systems Parametrized for All Spd-Block Elements (Z=1-86). J. Chem. Theory Comput. 2017, 13, 1989-2009.
(31) Bannwarth, C.; Ehlert, S.; Grimme, S. GFN2-XTB—An Accurate and Broadly Parametrized Self-Consistent Tight-Binding Quantum Chemical Method with Multipole Electrostatics and Density-Dependent Dispersion Contributions. J. Chem. Theory Comput. 2019, 15, 1652-1671.
(32) Neese, F. Software Update: The ORCA Program System, Version 4.0. Wiley Interdisciplinary Reviews: Computational Molecular Science 2018, 8, e1327.
(33) Neese, F. The ORCA Program System. WIREs Computational Molecular Science 2012, 2, 73-78.
(34) Becke, A. D. Density-Functional Exchange-Energy Approximation with Correct Asymptotic Behavior. Phys. Rev. A 1988, 38, 3098-3100.
(35) Lee, C.; Yang, W.; Parr, R. G. Development of the Colle-Salvetti Correlation-Energy Formula into a Functional of the Electron Density. Phys. Rev. B 1988, 37, 785-789.
(36) Becke, A. D. Density-functional Thermochemistry. Ill. The Role of Exact Exchange. J. Chem. Phys. 1993, 98, 5648-5652.
(37) Dunning, T. H. Gaussian Basis Sets for Use in Correlated Molecular Calculations. I. The Atoms Boron through Neon and Hydrogen. J. Chem. Phys. 1989, 90, 1007-1023.
(38) Casanova-Páez, M.; Dardis, M. B.; Goerigk, L. ΩB2PLYP and ΩB2GPPLYP: The First Two Double-Hybrid Density Functionals with Long-Range Correction Optimized for Excitation Energies. J. Chem. Theory Comput. 2019, 15, 4735-4744.
(39) Weigend, F.; Ahlrichs, R. Balanced Basis Sets of Split Valence, Triple Zeta Valence and Quadruple Zeta Valence Quality for H to Rn: Design and Assessment of Accuracy. Phys. Chem. Chem. Phys. 2005, 7, 3297-3305.
(40) Guo, Y.; Sivalingam, K.; Valeev, E. F.; Neese, F. SparseMaps—A Systematic Infrastructure for Reduced-Scaling Electronic Structure Methods. III. Linear-Scaling Multireference Domain-Based Pair Natural Orbital N-Electron Valence Perturbation Theory. J. Chem. Phys. 2016, 144, 094111.
(41) Nielsen, E. S.; Jo/rgensen, P.; Oddershede, J. Transition Moments and Dynamic Polarizabilities in a Second Order Polarization Propagator Approach. J. Chem. Phys. 1980, 73, 6238-6246.
(42) Sauer, S. P. A. Second-Order Polarization Propagator Approximation with Coupled-Cluster Singles and Doubles Amplitudes—SOPPA(CCSD): The Polarizability and Hyperpolarizability Of. J. Phys. B: At. Mol. Opt. Phys. 1997, 30, 3773-3780.
(43) Eriksen, J. J.; Sauer, S. P. A.; Mikkelsen, K. V.; Jensen, H. J. A.; Kongsted, J. On the Importance of Excited State Dynamic Response Electron Correlation in Polarizable Embedding Methods. Journal of Computational Chemistry 2012, 33, 2012-2022.
(44) Schirmer, J. Beyond the Random-Phase Approximation: A New Approximation Scheme for the Polarization Propagator. Phys. Rev. A 1982, 26, 2395-2416.
(45) Trofimov, A. B.; Schirmer, J. An Efficient Polarization Propagator Approach to Valence Electron Excitation Spectra. J. Phys. B: At. Mol. Opt. Phys. 1995, 28, 2299-2324.
(46) Starcke, J. H.; Wormit, M.; Dreuw, A. Unrestricted Algebraic Diagrammatic Construction Scheme of Second Order for the Calculation of Excited States of Medium-Sized and Large Molecules. J. Chem. Phys. 2009, 130, 024104.
(47) Wormit, M.; Rehn, D. R.; Harbach, P. H. P.; Wenzel, J.; Krauter, C. M.; Epifanovsky, E.; Dreuw, A. Investigating Excited Electronic States Using the Algebraic Diagrammatic Construction (ADC) Approach of the Polarisation Propagator. Molecular Physics 2014, 112, 774-784.
(48) ROWE, D. J. Equations-of-Motion Method and the Extended Shell Model. Rev. Mod. Phys. 1968, 40, 153-166.
(49) Emrich, K. An Extension of the Coupled Cluster Formalism to Excited States (I). Nuclear Physics A 1981, 351, 379-396.
(50) Geertsen, J.; Rittby, M.; Bartlett, R. J. The Equation-of-Motion Coupled-Cluster Method: Excitation Energies of Be and CO. Chemical Physics Letters 1989, 164, 57-62.
(51) Stanton, J. F.; Bartlett, R. J. The Equation of Motion Coupled-cluster Method. A Systematic Biorthogonal Approach to Molecular Excitation Energies, Transition Probabilities, and Excited State Properties. J. Chem. Phys. 1993, 98, 7029-7039.
(52) Krylov, A. I. Equation-of-Motion Coupled-Cluster Methods for Open-Shell and Electronically Excited Species: The Hitchhiker's Guide to Fock Space. Annu. Rev. Phys. Chem. 2008, 59, 433-462.
(53) Landau, A.; Khistyaev, K.; Dolgikh, S.; Krylov, A. I. Frozen Natural Orbitals for Ionized States within Equation-of-Motion Coupled-Cluster Formalism. J. Chem. Phys. 2010, 132, 014109.
(54) Sosa, C.; Geertsen, J.; Trucks, G. W.; Bartlett, R. J.; Franz, J. A. Selection of the Reduced Virtual Space for Correlated Calculations. An Application to the Energy and Dipole Moment of H2O. Chemical Physics Letters 1989, 159, 148-154.
(55) Taube, A. G.; Bartlett, R. J. Frozen Natural Orbitals: Systematic Basis Set Truncation for Coupled-Cluster Theory. Collect. Czech. Chem. Commun. 2005, 70, 837-850.
(56) Taube, A. G.; Bartlett, R. J. Frozen Natural Orbital Coupled-Cluster Theory: Forces and Application to Decomposition of Nitroethane. J. Chem. Phys. 2008, 128, 164101.
(57) Krylov, A. I. Spin-Flip Configuration Interaction: An Electronic Structure Model That Is Both Variational and Size-Consistent. Chemical Physics Letters 2001, 350, 522-530.
(58) Zhang, X.; Herbert, J. M. Spin-Flip, Tensor Equation-of-Motion Configuration Interaction with a Density-Functional Correction: A Spin-Complete Method for Exploring Excited-State Potential Energy Surfaces. J. Chem. Phys. 2015, 143, 234107.
(59) Perdew, J. P.; Burke, K.; Ernzerhof, M. Generalized Gradient Approximation Made Simple. Phys. Rev. Lett. 1996, 77, 3865-3868.
(60) Perdew, J. P.; Ernzerhof, M.; Burke, K. Rationale for Mixing Exact Exchange with Density Functional Approximations. J. Chem. Phys. 1996, 105, 9982-9985.
(61) Adamo, C.; Barone, V. Toward Reliable Density Functional Methods without Adjustable Parameters: The PBE0 Model. J. Chem. Phys. 1999, 110, 6158-6170.
(62) Bernard, Y. A.; Shao, Y.; Krylov, A. I. General Formulation of Spin-Flip Time-Dependent Density Functional Theory Using Non-Collinear Kernels: Theory, Implementation, and Benchmarks. The Journal of Chemical Physics 2012, 136, 204103.
(63) Shao, Y.; Gan, Z.; Epifanovsky, E.; Gilbert, A. T. B.; Wormit, M.; Kussmann, J.; Lange, A. W.; Behn, A.; Deng, J.; Feng, X.; et al. Advances in Molecular Quantum Chemistry Contained in the Q-Chem 4 Program Package. Molecular Physics 2015, 113, 184-215.
(64) Grimme, S.; Neese, F. Double-Hybrid Density Functional Theory for Excited Electronic States of Molecules. J. Chem. Phys. 2007, 127, 154116.
(65) Goerigk, L.; Moellmann, J.; Grimme, S. Computation of Accurate Excitation Energies for Large Organic Molecules with Double-Hybrid Density Functionals. Physical Chemistry Chemical Physics 2009, 11, 4611-4620.
(66) Goerigk, L.; Grimme, S. Double-Hybrid Density Functionals Provide a Balanced Description of Excited 1La and 1Lb States in Polycyclic Aromatic Hydrocarbons. J. Chem. Theory Comput. 2011, 7, 3272-3277.
(67) Schwabe, T.; Goerigk, L. Time-Dependent Double-Hybrid Density Functionals with Spin-Component and Spin-Opposite Scaling. J. Chem. Theory Comput. 2017, 13, 4307-4323.
(68) Shaw, J. T.; Prem, S. Fused S-Triazino Heterocycles. VI. 1,9,9b-Triazaphenalenes. Journal of Heterocyclic Chemistry 1977, 14, 671-672.
(69) Pratap, R.; Roy, A. D.; Kushwaha, S. P.; Goel, A.; Roy, R.; Ram, V. J. Guanidine and Amidine Mediated Synthesis of Bridgehead Triazaphenalenes, Pyrimidines and Pyridines through Domino Reactions. Tetrahedron Letters 2007, 48, 5845-5849.
(70) Matsuda, Y.; Gotou, H.; Katou, K.; Matsumoto, H. Studies on Quinolizine Derivatives. XXIII.: Synthesis and Reactions of Methylthioazacycl[3.3.3]Azines. Chemical & Pharmaceutical Bulletin 1989, 37, 1188-1191.
(71) Shaw, J. T.; Coffindaffer, T. W.; Stimmel, J. B.; Lindley, P. M. Fused-s-Triazino Heterocycles IX. 1,3,4,6,9b-Pentaazaphenalenes and 1,3,6,9b-Tetraazaphenalenes: Amino and Alkoxy Derivatives. Journal of Heterocyclic Chemistry 1982, 19, 357-361.
(72) Shaw, J. T.; Starkey, K. D.; Pelliccione, D. J.; Barnhart, S. L. Fused S-Triazino Heterocycles. X. Displacement Reactions of 7,9-Dibromo-2-Tribromomethyl-5-Trichloromethyl-1,3,4,6,9b-Pentaazaphenalene and 7,9-Dibromo-2,5-Bis(Tribromomethyl)-1,3,4,6,9b-Pentaazaphenalene. Journal of Heterocyclic Chemistry 1983, 20, 1095-1097.
(73) Matsuo, M.; Awaya, H.; Maseda, C.; Tominaga, Y.; Natsuki, R.; Matsuda, Y.; Kobayashi, G. Studies on Quinolizine Derivatives. XII. Synthesis of Diazacycl [3, 3, 3] Azine Derivatives. Chemical & Pharmaceutical Bulletin 1974, 22, 2765-2766.
(74) Rossman, M. A.; Leonard, N. J.; Urano, S.; LeBreton, P. R. Synthesis and Valence Orbital Structures of Azacycl[3.3.3]Azines in a Systematic Series. J. Am. Chem. Soc. 1985, 107, 3884-3890.
(75) Kanamori, K.; Roberts, J. D.; Rossman, M. A.; Leonard, N. J. Systematic Series of Azacycl[3.3.3]Azines of Varying Nitrogen Content: Nitrogen-15 Magnetic Resonance Spectra. Heteroatom Chemistry 1992, 3, 19-23.
(76) Ceder, O.; Vernmark, K. Synthesis of the 1,3,4-Triaza- and 1,4-Diazacycl[3.3.3]Azine Systems. Acta. Chem. Scand. B 1977, 31, 235-238.
(77) Boutique, J. P.; Verbist, J. J.; Fripiat, J. G.; Delhalle, J.; Pfister-Guillouzo, G.; Ashwell, G. J. 3,5,11,13-Tetraazacycl[3.3.3]Azine: Theoretical (Ab Initio) and Experimental (x-Ray and Ultraviolet Photoelectron Spectroscopy) Studies of the Electronic Structure. J. Am. Chem. Soc. 1984, 106, 4374-4378.
(78) Watanabe, H.; Hirose, M.; Tanaka, K.; Tanaka, K.; Chujo, Y. Color Tuning of Alternating Conjugated Polymers Composed of Pentaazaphenalene by Modulating Their Unique Electronic Structures Involving Isolated-LUMOs. Polym. Chem. 2016, 7, 3674-3680.
(79) Yeo, H.; Hirose, M.; Tanaka, K.; Chujo, Y. Construction of Multi-N-Heterocycle-Containing Organic Solvent-Soluble Polymers with 1,3,4,6,9b-Pentaazaphenalene. Polym J 2014, 46, 688-693.
(80) Watanabe, H.; Hirose, M.; Tanaka, K.; Chujo, Y. Development of Emissive Aminopentaazaphenalene Derivatives Employing a Design Strategy for Obtaining Luminescent Conjugated Molecules by Modulating the Symmetry of Molecular Orbitals with Substituent Effects. Chem. Commun. 2017, 53, 5036-5039.
(81) Watanabe, H.; Kawano, Y.; Tanaka, K.; Chujo, Y. Enhancing Light-Absorption and Luminescent Properties of Non-Emissive 1,3,4,6,9b-Pentaazaphenalene through Perturbation of Forbidden Electronic Transition by Boron Complexation. Asian Journal of Organic Chemistry 2020, 9, 259-266.
(82) Watanabe, H.; Tanaka, K.; Chujo, Y. Independently Tuned Frontier Orbital Energy Levels of 1,3,4,6,9b-Pentaazaphenalene Derivatives by the Conjugation Effect. J. Org. Chem. 2019, 84, 2768-2778.
(83) Winter, R. A. E.; Villani, T. J. U.S. Pat. No. 3,886,157-5,6,8,8B,9-Pentaazanaphth[3,2,1-d,e]Anthracene Derivatives. 3886157, May 27, 1975.
(84) Product Class 7: Cyclazines. In Category 2, Hetarenes and Related Ring Systems; Thieme Verlag, 2004.
(85) Murata, I.; Yamamoto, K.; Morioka, M.; Tamura, M.; Hirotsu, T. The Chemistry of Phenalenium Systems XXI. Cyclopenta[Cd]Phenalenyl Anion. Tetrahedron Letters 1975, 16, 2287-2288.
(86) Cunningham, R. P.; Farquhar, D.; Gibson, W. K.; Leaver, D. Heterocyclic Compounds with Bridgehead Nitrogen Atoms. Part IV. Cyclopenta[lj]Pyrido[2,1,6-de]Quinolizines (Cyclopenta[Cd]Cycl[3,3,3]-Azines). J. Chem. Soc. C 1969, No. 2, 239-243.
(87) Farquhar, D.; Gough, T. T.; Leaver, D. Heterocyclic Compounds with Bridgehead Nitrogen Atoms. Part V. Pyrido[2,1,6-de]Quinolizines (Cycl[3.3.3]Azines). J. Chem. Soc., Perkin Trans. 1 1976, No. 3, 341-355.
(88) Gibson, W. K.; Leaver, D. Synthesis of a Derivative of Cycl[3,3,3]Azine (9b-Azaphenalene). Chem. Commun. (London) 1965, No. 1, 11-11.
(89) Mamada, M.; Inada, K.; Komino, T.; Potscavage, W. J.; Nakanotani, H.; Adachi, C. Highly Efficient Thermally Activated Delayed Fluorescence from an Excited-State Intramolecular Proton Transfer System. ACS Cent. Sci. 2017, 3, 769-777.
(90) Kwon, J. E.; Park, S. Y. Advanced Organic Optoelectronic Materials: Harnessing Excited-State Intramolecular Proton Transfer (ESIPT) Process. Advanced Materials 2011, 23, 3615-3642.
(91) Wu, K.; Zhang, T.; Wang, Z.; Wang, L.; Zhan, L.; Gong, S.; Zhong, C.; Lu, Z.-H.; Zhang, S.; Yang, C. De Novo Design of Excited-State Intramolecular Proton Transfer Emitters via a Thermally Activated Delayed Fluorescence Channel. J. Am. Chem. Soc. 2018, 140, 8877-8886.
(92) Long, Y.; Mamada, M.; Li, C.; dos Santos, P. L.; Colella, M.; Danos, A.; Adachi, C.; Monkman, A. Excited State Dynamics of Thermally Activated Delayed Fluorescence from an Excited State Intramolecular Proton Transfer System. J. Phys. Chem. Lett. 2020, 11, 3305-3312.
(93) Cao, Y.; Eng, J.; Penfold, T. J. Excited State Intramolecular Proton Transfer Dynamics for Triplet Harvesting in Organic Molecules. J. Phys. Chem. A 2019, 123, 2640-2649.
(94) Padalkar, V. S.; Seki, S. Excited-State Intramolecular Proton-Transfer (ESIPT)-Inspired Solid State Emitters. Chem. Soc. Rev. 2015, 45, 169-202.
(95) Li, B.; Zhou, Q.; Sun, C.; Cao, B.; Li, Y.; Han, J.; Yin, H.; Shi, Y. Revised Excited-State Intramolecular Proton Transfer of the 3-Aminophthalimide Molecule: A TDDFT Study. Spectrochimica Acta Part A: Molecular and Biomolecular Spectroscopy 2020, 239, 118386.
(96) Jiang, G.; Li, F.; Fan, J.; Song, Y.; Wang, C.-K.; Lin, L. Theoretical Perspective for Luminescent Mechanism of Thermally Activated Delayed Fluorescence Emitter with Excited-State Intramolecular Proton Transfer. Journal of Materials Chemistry C 2020, 8, 98-108.
(97) Zhang, N.; Zhang, T.; Wen, L.; Wang, L.; Yan, J.; Zheng, K. Tuning the Excited-State Intramolecular Proton Transfer (ESIPT) Process of Indole-Pyrrole Systems by π-Conjugation and Substitution Effects: Experimental and Computational Studies. Physical Chemistry Chemical Physics 2020, 22, 1409-1415.
(98) Leupin, W.; Wirz, J. Low-Lying Electronically Excited States of Cycl[3.3.3]Azine, a Bridged 12.Pi.-Perimeter. J. Am. Chem. Soc. 1980, 102, 6068-6075.
(99) Ceder, Olof; Widing, Per-Olof; Vernmark, Karin, Synthesis of 1,9-Diazacycl[3.3.3]Azin. Acta Chem. Scand. 1976, 30b, 466-468.
(100) Ceder, O.; Vernmark, K. Synthesis of the 1,3,4-Triaza- and 1,4-Diazacycl[3.3.3]Azine Systems. Acta. Chem. Scand. B 1977, 31, 235-238.
(101) Leupin, W.; Magde, D.; Persy, G.; Wirz, J. 1,4,7-Triazacycl[3.3.3]Azine: Basicity, Photoelectron Spectrum, Photophysical Properties. J. Am. Chem. Soc. 1986, 108, 17-22.
(102) Ceder, Olof; Andersson, Johanna E., The Synthesis of 1,3,6-Triazacycl[3.3.3]Azines. Acta. Chem. Scand. B 1972, 26, 596-610.
(103) Rossman, M. A.; Leonard, N. J.; Urano, S.; LeBreton, P. R. Synthesis and Valence Orbital Structures of Azacycl[3.3.3]Azines in a Systematic Series. J. Am. Chem. Soc. 1985, 107, 3884-3890.
(104) Shahbaz, M.; Urano, S.; LeBreton, P. R.; Rossman, M. A.; Hosmane, R. S.; Leonard, N. J. Tri-s-Triazine: Synthesis, Chemical Behavior, and Spectroscopic and Theo¬retical Probes of Valence Orbital Structure. J. Am. Chem. Soc. 1984, 106, 2805-2811.
(105) Watanabe, H.; Tanaka, K.; Chujo, Y. Independently Tuned Frontier Orbital Energy Levels of 1,3,4,6,9b-Pentaazaphenalene Derivatives by the Conjugation Effect. J. Org. Chem. 2019, 84, 2768-2778.
(106) Grimme, S.; Neese, F. J. Chem. Phys. 2007, 127, 154116.
(107) Goerigk, L.; Moellmann, J.; Grimme, S. Phys. Chem. Chem. Phys. 2009, 11, 4611-4620.
(108) Goerigk, L.; Grimme, S. J. Chem. Theory Comput. 2011, 7, 3272-3277.
(109) Schwabe, T.; Goerigk, L. J. Chem. Theory Comput. 2017, 13, 4307-4323.
(110) Casanova-Páez, M.; Dardis, M. B.; Goerigk, L. J. Chem. Theory Comput. 2019, 15, 4735-4744.
(111) Krylov, A. I. Chem. Phys. Lett. 2001, 350, 522-530.
(112) Zhang, X.; Herbert, J. M. J. Chem. Phys. 2015, 143, 234107.
(113) Perdew, J. P.; Burke, K.; Ernzerhof, M. Phys. Rev. Lett. 1996, 77, 3865-3868.
(114) Perdew, J. P.; Ernzerhof, M.; Burke, K. J. Chem. Phys. 1996, 105, 9982-9985.
(115) Adamo, C.; Barone, V. J. Chem. Phys. 1999, 110, 6158-6170.
(116) Bernard, Y. A.; Shao, Y.; Krylov, A. I. J. Chem. Phys. 2012, 136, 204103.
(117) Casanova-Páez, M.; Goerigk, L. J. Chem. Phys. 2020, 153, 064106.
(118) de Souza, B.; Neese, F.; Izsák, R. J. Chem. Phys. 2018, 148, 034104.
(119) Baiardi, A.; Bloino, J.; Barone, V. J. Chem. Theory Comput. 2013, 9, 4097-4115.
(120) Pracht, P.; Grimme, S. Conformer-Rotamer Ensemble Sampling Tool. https://github.com/grimme-lab/crest (accessed Jun. 4, 2020).
(121) Grimme, S. J. Chem. Theory Comput. 2019, 15, 2847-2862.
(122) Pracht, P.; Bohle, F.; Grimme, S. Phys. Chem. Chem. Phys. 2020, 22, 7169-7192.
(123) Pracht, P.; Caldeweyher, E.; Ehlert, S.; Grimme, S. ChemRxiv 2019.
(124) Grimme, S. Semiempirical Extended Tight-Binding Program Package https://github.com/grimme-lab/xtb (accessed Jun. 4, 2020).
(125) Grimme, S.; Bannwarth, C.; Shushkov, P. J. Chem. Theory Comput. 2017, 13, 1989-2009.
(126) Bannwarth, C.; Ehlert, S.; Grimme, S. J. Chem. Theory Comput. 2019, 15, 1652-1671.
(127) Neese, F. WIREs Comput. Mol. Sci. 2018, 8, e1327.
(128) Neese, F. WIREs Comput. Mol. Sci. 2012, 2, 73-78.
(129) Becke, A. D. Phys. Rev. A 1988, 38, 3098-3100.
(130) Lee, C.; Yang, W.; Parr, R. G. Phys. Rev. B 1988, 37, 785-789.
(131) Becke, A. D. J. Chem. Phys. 1993, 98, 5648-5652.
(132) Dunning, T. H. J. Chem. Phys. 1989, 90, 1007-1023.
(133) Weigend, F.; Ahlrichs, R. Phys. Chem. Chem. Phys. 2005, 7, 3297-3305.
(134) Guo, Y.; Sivalingam, K.; Valeev, E. F.; Neese, F. J. Chem. Phys. 2016, 144, 094111.
(135) Nielsen, E. S.; Jo/rgensen, P.; Oddershede, J. J. Chem. Phys. 1980, 73, 6238-6246.
(136) Sauer, S. P. A. J. Phys. B: At. Mol. Opt. Phys. 1997, 30, 3773-3780.
(137) Eriksen, J. J.; Sauer, S. P. A.; Mikkelsen, K. V.; Jensen, H. J. A.; Kongsted, J. J. Comput. Chem. 2012, 33, 2012-2022.
(138) Schirmer, J. Phys. Rev. A 1982, 26, 2395-2416.
(139) Trofimov, A. B.; Schirmer, J. J. Phys. B: At. Mol. Opt. Phys. 1995, 28, 2299-2324.
(140) Starcke, J. H.; Wormit, M.; Dreuw, A. J. Chem. Phys. 2009, 130, 024104.
(141) Wormit, M.; Rehn, D. R.; Harbach, P. H. P.; Wenzel, J.; Krauter, C. M.; Epifanovsky, E.; Dreuw, A. Mol. Phys. 2014, 112, 774-784.
(142) Kendall, R. A.; Dunning, T. H.; Harrison, R. J. J. Chem. Phys. 1992, 96, 6796-6806.
(143) Casanova, D.; Rhee, Y. M.; Head-Gordon, M. J. Chem. Phys. 2008, 128, 164106.
(144) Hättig, C.; Weigend, F. J. Chem. Phys. 2000, 113, 5154-5161.
(145) Hättig, C.; Hald, K. Phys. Chem. Chem. Phys. 2002, 4, 2111-2118.
(146) Head-Gordon, M.; Rico, R. J.; Oumi, M.; Lee, T. J. Chemical Physics Letters 1994, 219, 21-29.
(147) Head-Gordon, M.; Maurice, D.; Oumi, M. Chemical Physics Letters 1995, 246, 114-121.
(148) ROWE, D. J. Rev. Mod. Phys. 1968, 40, 153-166.
(149) Emrich, K. Nuclear Physics A 1981, 351, 379-396.
(150) Geertsen, J.; Rittby, M.; Bartlett, R. J. Chemical Physics Letters 1989, 164, 57-62.
(151) Stanton, J. F.; Bartlett, R. J. J. Chem. Phys. 1993, 98, 7029-7039.
(152) Krylov, A. I. Annu. Rev. Phys. Chem. 2008, 59, 433-462.
(153) Landau, A.; Khistyaev, K.; Dolgikh, S.; Krylov, A. I. J. Chem. Phys. 2010, 132, 014109.
(154) Sosa, C.; Geertsen, J.; Trucks, G. W.; Bartlett, R. J.; Franz, J. A. Chemical Physics Letters 1989, 159, 148-154.
(155) Taube, A. G.; Bartlett, R. J. Collect. Czech. Chem. Commun. 2005, 70, 837-850.
(156) Taube, A. G.; Bartlett, R. J. J. Chem. Phys. 2008, 128, 164101.
(157) Shao, Y.; Gan, Z.; Epifanovsky, E.; Gilbert, A. T. B.; Wormit, M.; Kussmann, J.; Lange, A. W.; Behn, A.; Deng, J.; Feng, X.; et al. Mol. Phys. 2015, 113, 184-215.
(158) Furche, F.; Ahlrichs, R.; Hättig, C.; Klopper, W.; Sierka, M.; Weigend, F. WIREs Comput. Mol. Sci. 2014, 4, 91-100.
(159) Balasubramani, S. G.; Chen, G. P.; Coriani, S.; Diedenhofen, M.; Frank, M. S.; Franzke, Y. J.; Furche, F.; Grotjahn, R.; Harding, M. E.; Hättig, C.; et al. J. Chem. Phys. 2020, 152, 184107.
(160) Barone, V.; Cossi, M. J. Phys. Chem. A 1998, 102, 1995-2001.
(161) Cancès, E.; Mennucci, B.; Tomasi, J. J. Chem. Phys. 1997, 107, 3032-3041.
(162) Chipman, D. M. J. Chem. Phys. 2000, 112, 5558-5565.
(163) Torabi, S.; Jahani, F.; Severen, I. V.; Kanimozhi, C.; Patil, S.; Havenith, R. W. A.; Chiechi, R. C.; Lutsen, L.; Vanderzande, D. J. M.; Cleij, T. J.; et al. Advanced Functional Materials 2015, 25, 150-157.
(164) Wang, C.; Zhang, Z.; Pejić, S.; Li, R.; Fukuto, M.; Zhu, L.; Sauvé, G. Macromolecules 2018, 51, 9368-9381.
(165) Salehi, A.; Ho, S.; Chen, Y.; Peng, C.; Yersin, H.; So, F. Advanced Optical Materials 2017, 5, 1700197.
(166) Salehi, A.; Chen, Y.; Fu, X.; Peng, C.; So, F. ACS Appl. Mater. Interfaces 2018, 10, 9595-9601.
(167) Mei, G.; Wu, D.; Ding, S.; Choy, W. C. H.; Wang, K.; Sun, X. W. IEEE Photonics Journal 2020, 12, 1-14.
(168) Humeniuk, A.; Buz̆anc̆ić, M.; Hoche, J.; Cerezo, J.; Mitrić, R.; Santoro, F.; Bonac̆ić-Koutecký, V. J. Chem. Phys. 2020, 152, 054107.
(169) Charaf-Eddin, A.; Planchat, A.; Mennucci, B.; Adamo, C.; Jacquemin, D. J. Chem. Theory Comput. 2013, 9, 2749-2760.
(170) Ditchfield, R.; Hehre, W. J.; Pople, J. A. J. Chem. Phys. 1971, 54, 724-728.
(171) Hehre, W. J.; Ditchfield, R.; Pople, J. A. J. Chem. Phys. 1972, 56, 2257-2261.
(172) Hariharan, P. C.; Pople, J. A. Theoret. Chim. Acta 1973, 28, 213-222.
(173) Casida, M. E.; Huix-Rotllant, M. Annual Review of Physical Chemistry 2012, 63, 287-323.
(174) Petrenko, T.; Neese, F. J. Chem. Phys. 2012, 137, 234107.
(175) Niu, Y.; Peng, Q.; Deng, C.; Gao, X.; Shuai, Z. J. Phys. Chem. A 2010, 114, 7817-7831.
(176) Neumann, T.; Danilov, D.; Lennartz, C.; Wenzel, W. Journal of Computational Chemistry 2013, 34, 2716-2725.
(177) Friederich, P.; Symalla, F.; Meded, V.; Neumann, T.; Wenzel, W. J. Chem. Theory Comput. 2014, 10, 3720-3725.

1. A compound of Formula I: 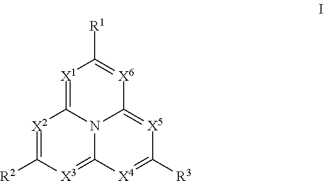
wherein
X¹ is selected from N and CR⁴;
X² is selected from N and CR⁵;
X³ is selected from N and CR⁶;
X⁴ is selected from N and CR⁷;
X⁵ is selected from N and CR⁸;
X⁶ is selected from N and CR⁹;
provided that at least one, but not all, of X¹-X⁶ is N;
R¹-R⁹ are independently selected from H, halo, NO₂, CN, isonitrile, C(O)H, NH₂, OH, SH, C(O)NH₂, C₁₋₁₀alkyl, C₃₋₁₀cycloalkyl, C₂₋₁₀alkenyl, C₂₋₁₀alkynyl, OC₁₋₁₀alkyl, NHC₁₋₁₀alkyl, NH(C₃₋₁₀cycloalkyl), N(C₁₋₁₀alkyl)(C₁₋₁₀alkyl), 3- to 8-membered heterocycloalkyl, C(O)C₁₋₁₀alkyl, CO₂C₁₋₁₀alkyl, C(O)NHC₁₋₁₀alkyl, C(O)N(C₁₋₁₀alkyl)(C₁₋₁₀alkyl), SC₁₋₁₀alkyl, S(O)C₁₋₁₀alkyl, SO₂C₁₋₁₀alkyl, OC(O)C₁₋₁₀alkyl, NHC(O)C₁₋₁₀alkyl, aryl, O-aryl, NH-aryl, N(aryl)(aryl), S-aryl, S(O)-aryl, OSO₂C₁₋₁₀alkyl, SO₂-aryl, C(O)-aryl; CO₂-aryl, C(O)NH-aryl, OC(O)-aryl, NHC(O)-aryl, heteroaryl, O-heteroaryl, NH-heteroaryl, S-heteroaryl, S(O)-heteroaryl, SO₂-heteroaryl, C(O)-heteroaryl, C(O)NH₂, CO₂-heteroaryl, C(O)NH— heteroaryl, OC(O)C₁₋₁₀alkyl, OC(O)-heteroaryl and NHC(O)-heteroaryl, wherein all alkyl, cycloalkyl, alkenyl, alkynyl, aryl, heterocycloalkyl, and heteroaryl groups are each unsubstituted or substituted with one or more substituents independently selected from R¹⁰;
or optionally, R¹ to R⁵, R⁸ and R⁹ are as defined above, R⁶ and R⁷ are linked to form X⁷═X⁸, which, together with X³, X⁴ and the carbon atom therebetween, form a five membered ring;
X⁷ is selected from N and CR¹¹;
X⁸ is selected from N and CR¹²;
optionally, R² and R¹¹ and/or R³ and R¹² together with the atoms therebetween are linked to form a 5- or 6-membered carbocycle or heterocycle, optionally an aromatic or heteroaromatic cycle, wherein the 5- or 6-membered carbocycle or heterocycle is unsubstituted or substituted with one or more substituents independently selected from R¹⁰;
or optionally, R¹, R⁴, R⁵, R⁸ and R⁹ are as defined above, R² and R⁶ and/or R³ and R⁷ together with the atoms therebetween are linked to form a 5- or 6-membered carbocycle or heterocycle, optionally an aromatic or heteroaromatic cycle, wherein the 5- or 6-membered carbocycle or heterocycle is unsubstituted or substituted with one or more substituents independently selected from R¹⁰;
R¹⁰ is selected from halo, NO₂, CN, isonitrile, C(O)H, NH₂, OH, SH, BH₂, C₁₋₆alkyl boronic ester, C₁₋₆alkyl borane, diaryl borane, C₂₋₆alkyldiol cyclic boronic ester, C(O)NH₂, C₃₋₁₀cycloalkyl, C₁₋₁₀alkyl, C₂₋₁₀alkenyl, C₂₋₁₀alkynyl, OC₁₋₁₀alkyl, NHC₁₋₁₀alkyl, N(C₁₋₁₀alkyl)(C₁₋₁₀alkyl), N(aryl)(aryl), NH(C₃₋₁₀cycloalkyl), 3- to 8-membered heterocycloalkyl, C(O)C₁₋₁₀alkyl, CO₂C₁₋₁₀alkyl, C(O)NHC₁₋₁₀alkyl, C(O)N(C₁₋₁₀alkyl)(C₁₋₁₀alkyl), SC₁₋₁₀alkyl, S(O)C₁₋₁₀alkyl, SO₂C₁₋₁₀alkyl, OC(O)C₁₋₁₀alkyl, NHC(O)H, NHC(O)C₁₋₁₀alkyl, aryl, O-aryl, NH-aryl, S-aryl, S(O)-aryl, SO₂-aryl, C(O)-aryl; CO₂-aryl, C(O)NH-aryl, OC(O)-aryl, NHC(O)-aryl, heteroaryl, O-heteroaryl, NH-heteroaryl, S-heteroaryl, S(O)-heteroaryl, SO₂-heteroaryl, C(O)-heteroaryl; CO₂-heteroaryl, C(O)NH-heteroaryl, OC(O)-heteroaryl and NHC(O)-heteroaryl, wherein all alkyl, cycloalkyl, heterocycloalkyl, alkenyl, alkynyl, aryl, heterocycle, and heteroaryl groups are each unsubstituted or substituted with one or more substituents independently selected from halo, NO₂, CN, NH₂, OH, C₃₋₁₀cycloalkyl, C₁₋₁₀alkyl, OC₁₋₁₀alkyl, NHC₁₋₁₀alkyl, N(C₁₋₁₀alkyl)(C₁₋₁₀alkyl), NH(C₃₋₁₀cycloalkyl), trialkylsilanyl, C(O)aryl, aryl, heteroaryl, O-heteroaryl, N-heteroaryl, and S-heteroaryl;
R¹¹ and R¹² are independently selected from H, halo, NO₂, CN, C(O)H, NH₂, OH, SH, C(O)NH₂, C₁₋₁₀alkyl, C₂₋₁₀alkenyl, C₂₋₁₀alkynyl, OC₁₋₁₀alkyl, NHC₁₋₁₀alkyl, N(C₁₋₁₀alkyl)(C₁₋₁₀alkyl), C(O)C₁₋₁₀alkyl, CO₂C₁₋₁₀alkyl, C(O)NHC₁₋₁₀alkyl, C(O)N(C₁₋₁₀alkyl)(C₁₋₁₀alkyl), SC₁₋₁₀alkyl, S(O)C₁₋₁₀alkyl, SO₂C₁₋₁₀alkyl, OC(O)C₁₋₁₀alkyl, NHC(O)C₁₋₁₀alkyl, aryl, O-aryl, NH-aryl, S-aryl, S(O)-aryl, SO₂-aryl, C(O)-aryl; CO₂-aryl, C(O)NH-aryl, OC(O)-aryl, NHC(O)-aryl, heteroaryl, O-heteroaryl, NH-heteroaryl, S-heteroaryl, S(O)-heteroaryl, SO₂-heteroaryl, C(O)-heteroaryl; CO₂-heteroaryl, C(O)NH-heteroaryl, OC(O)-heteroaryl and NHC(O)-heteroaryl, wherein all alkyl, alkenyl, alkynyl, aryl and heteroaryl groups are each unsubstituted or substituted with one or more substituents independently selected from R¹³;
R¹³ is selected from halo, NO₂, CN, isonitrile, C(O)H, NH₂, OH, SH, C(O)NH₂, C₁₋₁₀alkyl, C₂₋₁₀alkenyl, C₂₋₁₀alkynyl, OC₁₋₁₀alkyl, NHC₁₋₁₀alkyl, N(C₁₋₁₀alkyl)(C₁₋₁₀alkyl), C(O)C₁₋₁₀alkyl, CO₂C₁₋₁₀alkyl, C(O)NHC₁₋₁₀alkyl, C(O)N(C₁₋₁₀alkyl)(C₁₋₁₀alkyl), SC₁₋₁₀alkyl, S(O)C₁₋₁₀alkyl, SO₂C₁₋₁₀alkyl, OC(O)C₁₋₁₀alkyl, NHC(O)C₁₋₁₀alkyl, aryl, O-aryl, NH-aryl, S-aryl, S(O)-aryl, SO₂-aryl, C(O)-aryl; CO₂-aryl, C(O)NH-aryl, OC(O)-aryl, NHC(O)-aryl, heteroaryl, O-heteroaryl, NH-heteroaryl, S-heteroaryl, S(O)-heteroaryl, SO₂-heteroaryl, C(O)-heteroaryl; CO₂-heteroaryl, C(O)NH-heteroaryl, OC(O)-heteroaryl and NHC(O)-heteroaryl;
all available H atoms are each optionally fluoro-substituted;
wherein the compound has a negative singlet-triple gap and an oscillator strength greater than or equal to about 0.01.

2. The compound of claim 1, wherein 2 to 4 of X¹ to X⁶ are N.

3. The compound of claim 1 or 2, wherein each halo is independently selected from F, Br, and Cl.

4. The compound of any one of claims 1 to 3, wherein each C₁₋₁₀alkyl is independently selected from linear and branched C₁₋₆alkyl.

5. The compound of claim 4, wherein the linear and branched C₁₋₆alkyl is selected from methyl, ethyl, propyl, butyl, isopropyl, secpropyl, secbutyl, and tertbutyl.

6. The compound of any one of claims 1 to 5, wherein each heterocycle and heterocyclocycloalkyl is independently selected from azetidine, aziridine, pyrrolidine, pipperidine, morpholine, tetrahydrofuran, tetrahydropyran, tetrahydrothiopyran, indolinone, and quinolinone.

7. The compound of any one of claims 1 to 6, wherein each aryl is independently selected from phenyl and naphthyl.

8. The compound of any one of claims 1 to 7, wherein each heterocycle and heteroaryl is independently selected from pyrrole, pyrazole, pyridine, indole, carbazole, indazole, imidazole, oxazole, isoxazole, thiazole, thiophene, furan, pyridazine, isothiazole, pyrimidine, benzofuran, benzothiophene, benzoimidazole, and quinoline.

9. The compound of claim 1 or 2, wherein R¹-R⁹ are independently selected from H, F, Br, Cl, NO₂, CN, isonitrile, C(O)H, NH₂, OH, SH, C₁₋₆alkyl, C₃₋₈cycloalkyl, C₂₋₄alkenyl, C₂₋₄alkynyl, OC₁₋₆alkyl, NHC₁₋₆alkyl, N(C₁₋₆alkyl)(C₁₋₆alkyl), C(O)C₁₋₆alkyl, SC₁₋₆alkyl, S(O)C₁₋₆alkyl, OC(O)C₁₋₆alkyl, aryl, N(aryl)(aryl), S-aryl, heteroaryl, C(O)NH₂.

10. The compound of claim 9, wherein R¹-R⁹ are independently selected from H, F, Br, Cl, NO₂, CN, isonitrile, C(O)H, NH₂, OH, SH, CF₃, methyl, ethyl, propyl, butyl, isopropyl, secpropyl, secbutyl, tertbutyl, C₃₋₆cycloalkyl, CH═CH₂, C≡CH, OCH₃, OEt, Oisopropyl, Otertbutyl, OCF₃, NHCH₃, NHCH₂CH₃, NHisopropyl, NHtertbutyl, N(CH₃)₂, NH(CH₂CH₃)₂, C(O)CH₃, C(O)CH₂CH₃, SCH₃, SCH₂CH₃, S(O)CH₃, S(O)CH₂CH₃, OC(O)CH₃, OC(O)CH₂CH₃, phenyl, naphthyl, N(phenyl)(phenyl), S-phenyl, S-naphthyl, NH-phenyl, O-pehynl, pyrrole, pyrazole, indole, indazole, benzoimidazole, pyridine, carbazole, benzofuran, benzothiophene, furan, thiophene, imidazole, oxazole, isoxazole, thiazole, C(O)NH₂.

11. The compound of any one of claims 1, 2, 9 and 10, wherein R¹⁰ is selected from F, Br, Cl, NO₂, CN, NH₂, OH, SH, C₁₋₆alkyl, OC₁₋₆alkyl, NHC₁₋₆alkyl, N(C₁₋₆alkyl)(C₁₋₆alkyl), N(aryl)(aryl), NH(C₃₋₁₀cycloalkyl), 3- to 8-membered heterocycloalkyl, NHC(O)H, NHC(O)C₁₋₆alkyl, aryl, NH-aryl, C(O)-aryl, heteroaryl, NH-heteroaryl, wherein all alkyl, cycloalkyl, alkenyl, alkynyl, aryl, C₁₋₁₀akyl substituted aryl, heterocycle, and heteroaryl groups are each unsubstituted or substituted with one or more substituents independently selected from halo, NO₂, CN, NH₂, OH, C₃₋₆cycloalkyl, C₁₋₆alkyl, OC₁₋₆alkyl, N(C₁₋₆alkyl)(C₁₋₆alkyl), trialkylsilanyl, heteroaryl.

12. The compound of any one of claims 1, 2, and 9 to 11, wherein R¹⁰ is selected from F, Br, Cl, NO₂, CN, NH₂, OH, SH, CF₃, methyl, ethyl, propyl, butyl, isopropyl, secpropyl, secbutyl, tertbutyl, OCH₃, OEt, Oisopropyl, Otertbutyl, OCF₃, NHCH₃, NHCH₂CH₃, NHisopropyl, NHtertbutyl, N(CH₃)₂, N(isopropyl)₂, N(phenyl)(phenyl), NH(C₃₋₆cycloalkyl), azetidine, aziridine, pyrrolidine, pipperidine, morpholine, tetrahydrofuran, tetrahydropyran, tetrahydrothiopyran, NHC(O)H, NHC(O)CH₃, NHC(O)CH₂CH₃, phenyl, naphthyl, NH-phenyl, NH-naphthyl, C(O)-phenyl, pyrrole, imidazole, pyrazole, carbazole, indole, NH-pyridine, NH-pyrrole, NH-furan, NH-imidazole, NH-thiophene, NH-pyridazine, NH-pyrimidine, NH-isoxazole, NH-oxazole, NH-pyrazole, NH-isothiazole, NH-thiazole, NH-indole, wherein all alkyl, cycloalkyl, alkenyl, alkynyl, aryl, heterocycle, and heteroaryl groups are each unsubstituted or substituted with one or more substituents independently selected from F, NO₂, CN, NH₂, OH, C₃₋₆cycloalkyl, methyl, ethyl, propyl, butyl, isopropyl, secpropyl, secbutyl, tertbutyl, OCH₃, OEt, N(CH₃)₂, N(CH₂CH₃)₂, triethylsilanyl, trimethylsilanyl phenyl, pyrazine.

13. The compound of any one of claims 1 to 12, wherein the compound is selected from

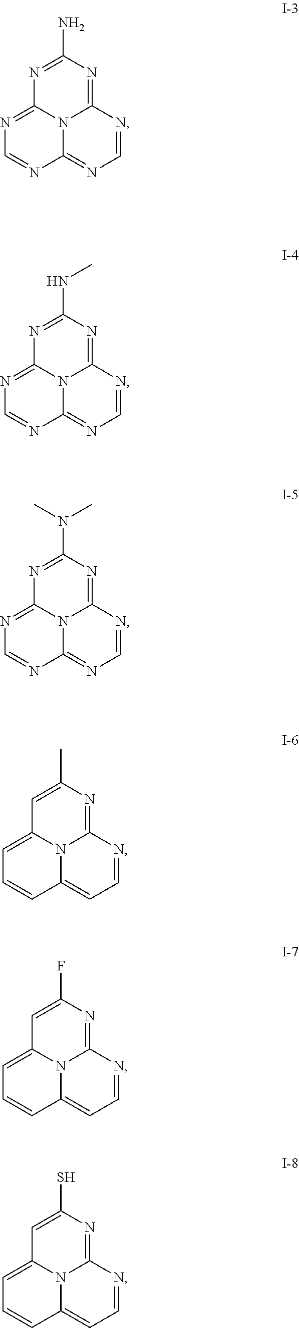 [SMILES: NC1=NC2=NC=NC3=NC=NC(=N1)N32 ; CNC1=NC2=NC=NC3=NC=NC(=N1)N32 ; CN(C)C1=NC2=NC=NC3=NC=NC(=N1)N32 ; CC1=CC2=CC=CC3=CC=NC(=N1)N32 ; FC1=CC2=CC=CC3=CC=NC(=N1)N32 ; SC1=CC2=CC=CC3=CC=NC(=N1)N32]

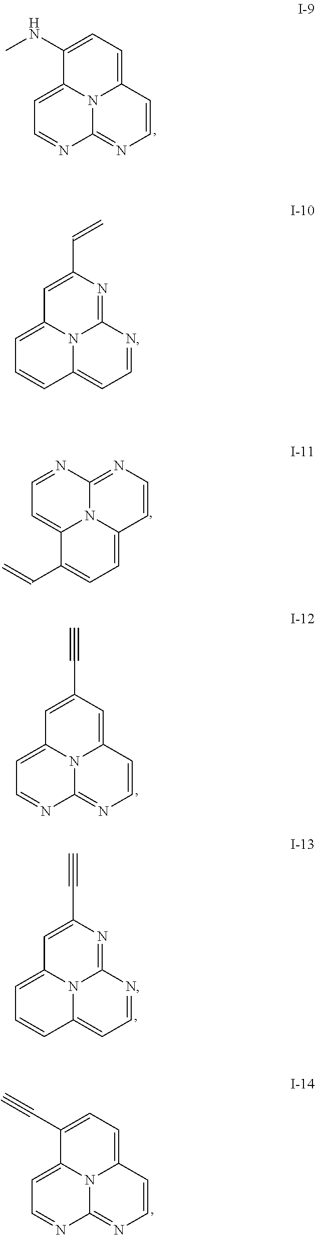

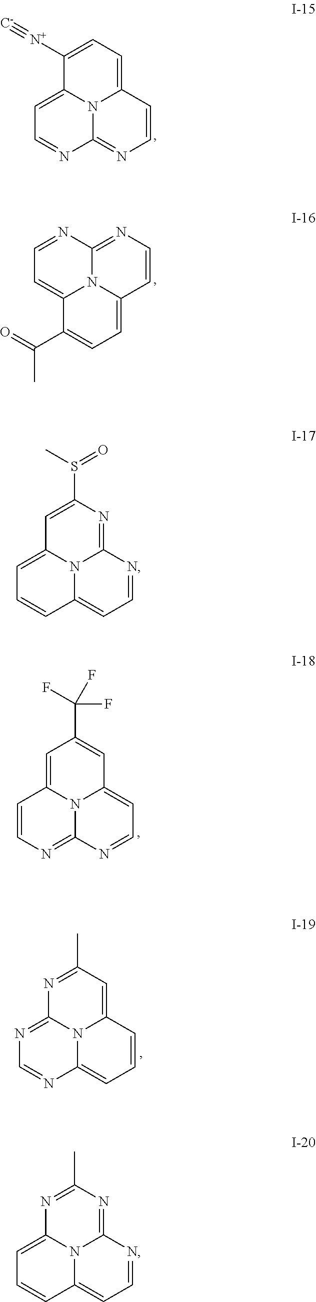 [SMILES: [C-]#[N+]C1=CC=C2C=CN=C3N=CC=C1N23 ; CC(=O)C1=CC=C2C=CN=C3N=CC=C1N23 ; CS(=O)C1=CC2=CC=CC3=CC=NC(=N1)N32 ; FC(F)(F)C1=CC2=CC=NC3=NC=CC(=C1)N23 ; CC1=NC2=NC=NC3=CC=CC(=C1)N32 ; CC1=NC2=CC=CC3=CC=NC(=N1)N32]

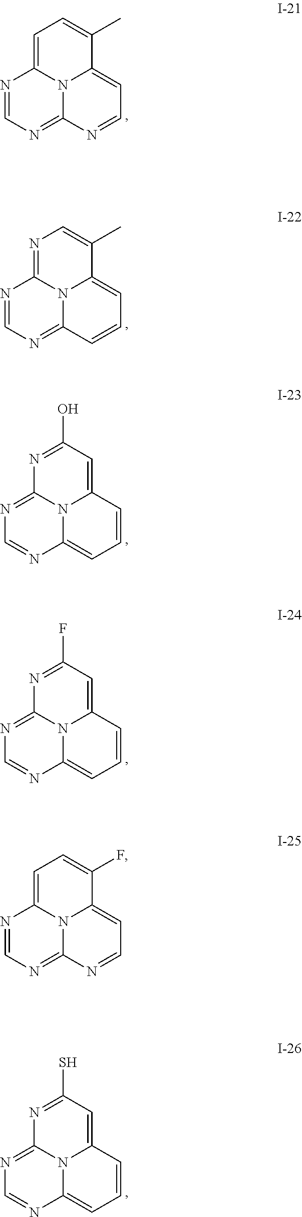

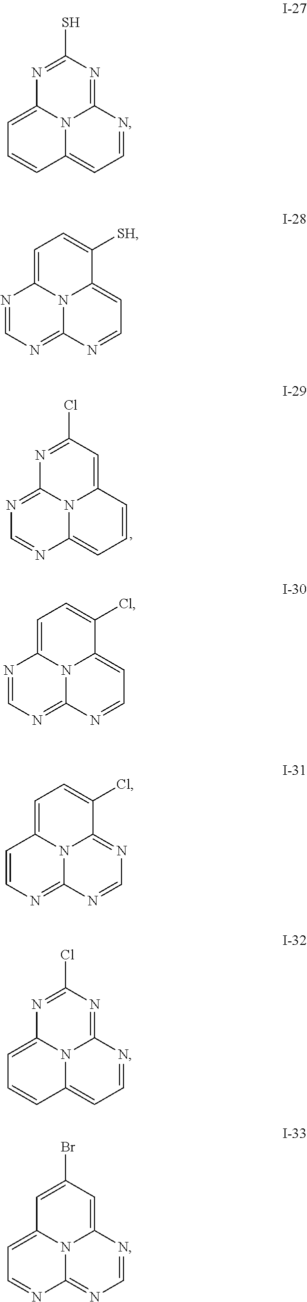

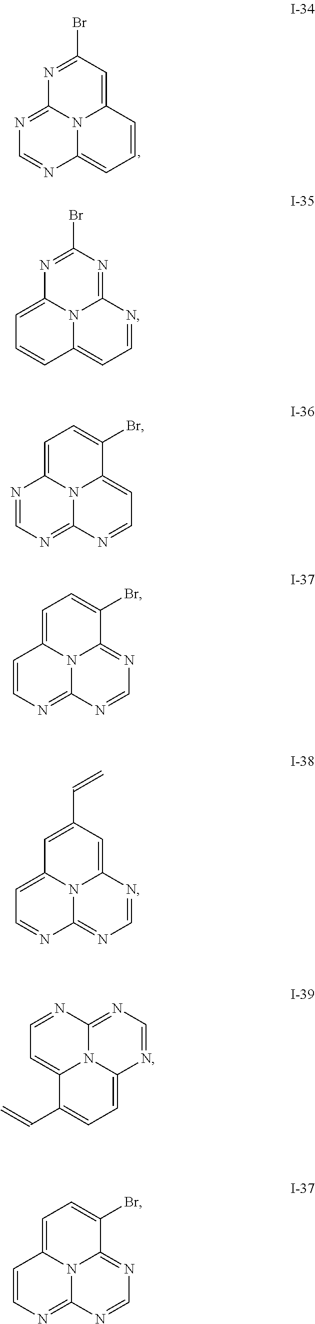

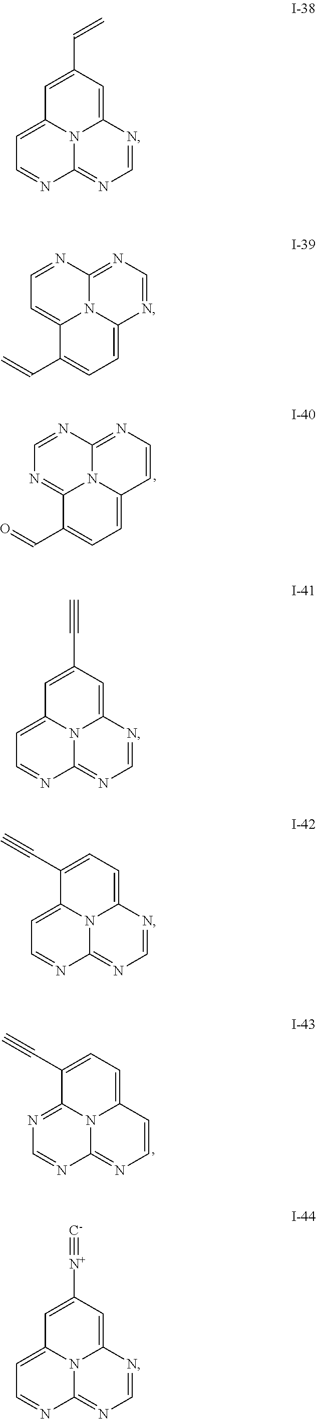

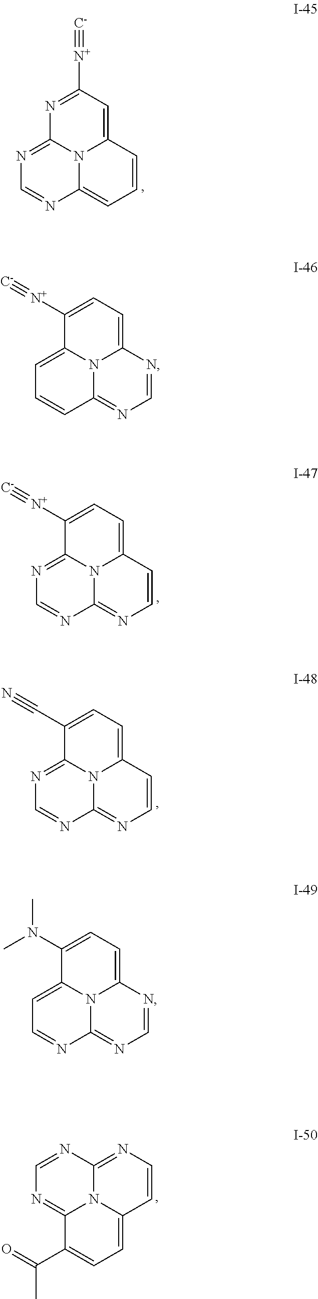 [SMILES: [C-]#[N+]C1=NC2=NC=NC3=CC=CC(=C1)N32 ; [C-]#[N+]C1=CC=C2N=CN=C3C=CC=C1N23 ; [C-]#[N+]C1=CC=C2C=CN=C3N=CN=C1N23 ; N#CC1=CC=C2C=CN=C3N=CN=C1N23 ; CN(C)C1=CC=C2N=CN=C3N=CC=C1N23 ; CC(=O)C1=CC=C2C=CN=C3N=CN=C1N23]

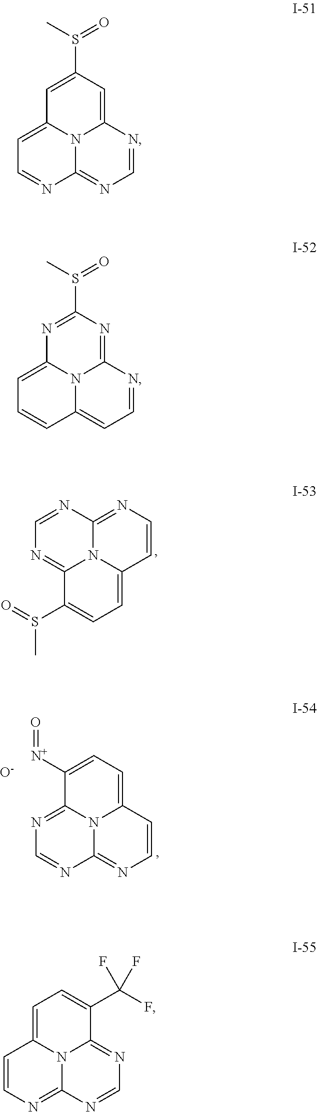

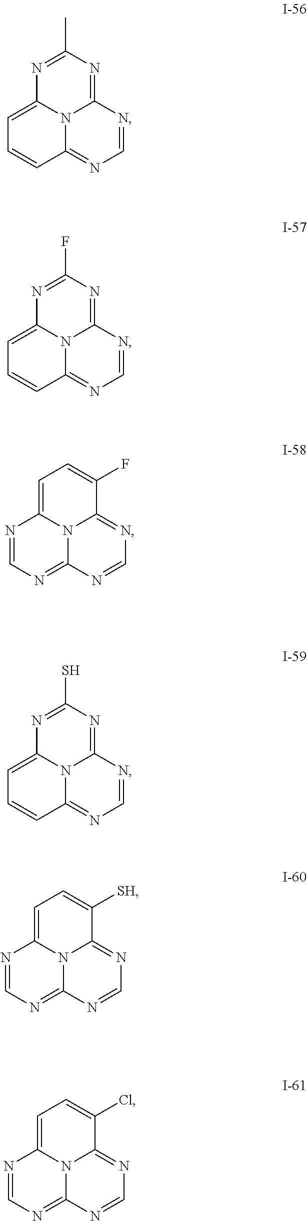

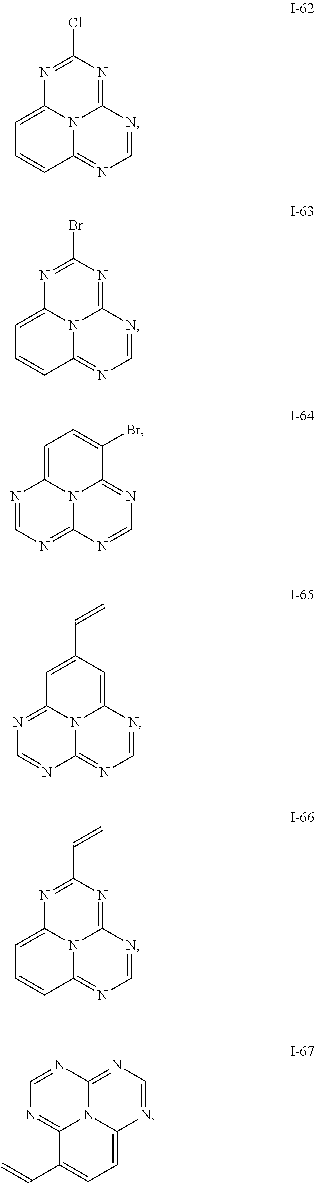

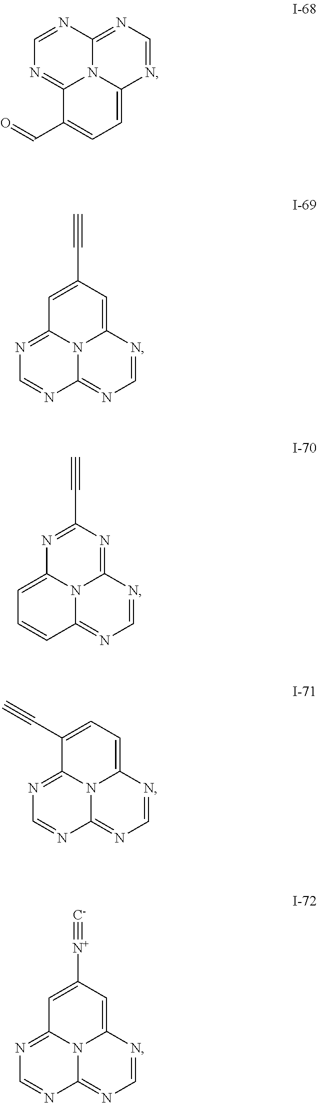

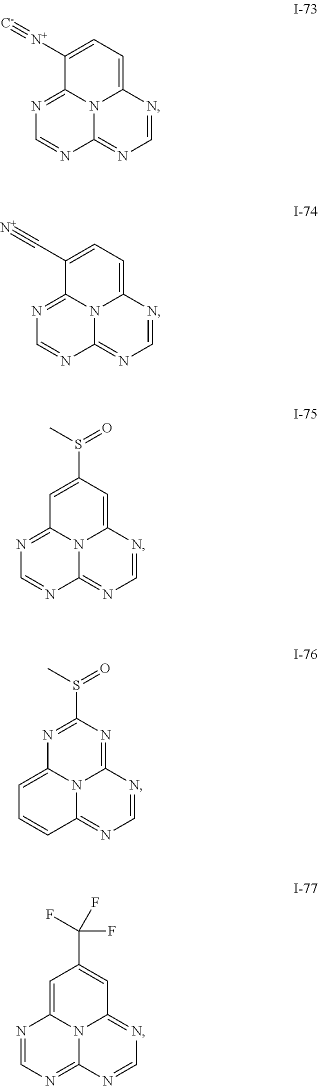 [SMILES: [C-]#[N+]C1=CC=C2N=CN=C3N=CN=C1N23 ; [NH+]#CC1=CC=C2N=CN=C3N=CN=C1N23 ; CS(=O)C1=CC2=NC=NC3=NC=NC(=C1)N23 ; CS(=O)C1=NC2=CC=CC3=NC=NC(=N1)N23 ; FC(F)(F)C1=CC2=NC=NC3=NC=NC(=C1)N23]

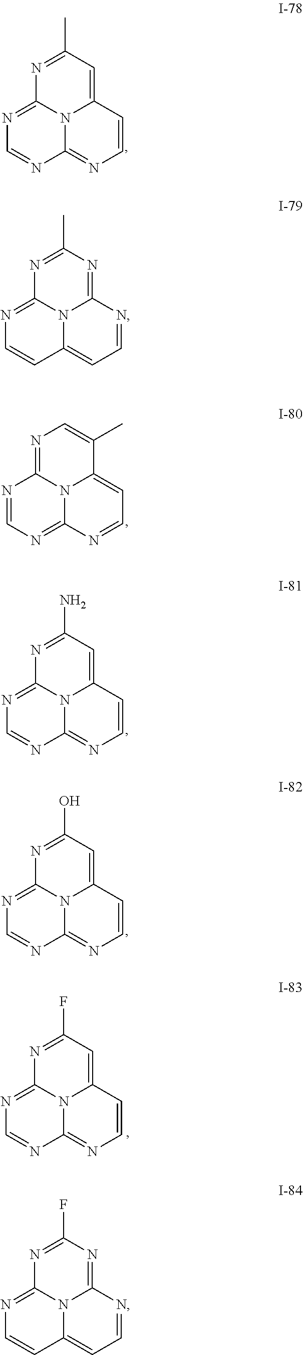

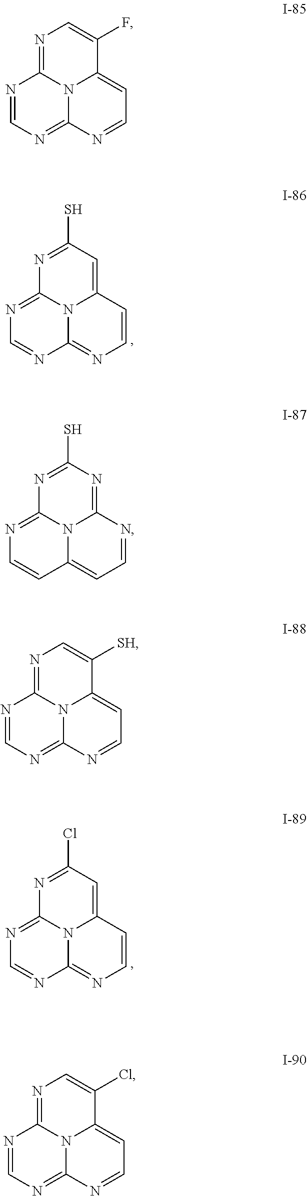 [SMILES: FC1=CN=C2N=CN=C3N=CC=C1N32 ; SC1=NC2=NC=NC3=NC=CC(=C1)N32 ; SC1=NC2=NC=CC3=CC=NC(=N1)N32 ; SC1=CN=C2N=CN=C3N=CC=C1N32 ; ClC1=NC2=NC=NC3=NC=CC(=C1)N32 ; ClC1=CN=C2N=CN=C3N=CC=C1N32]

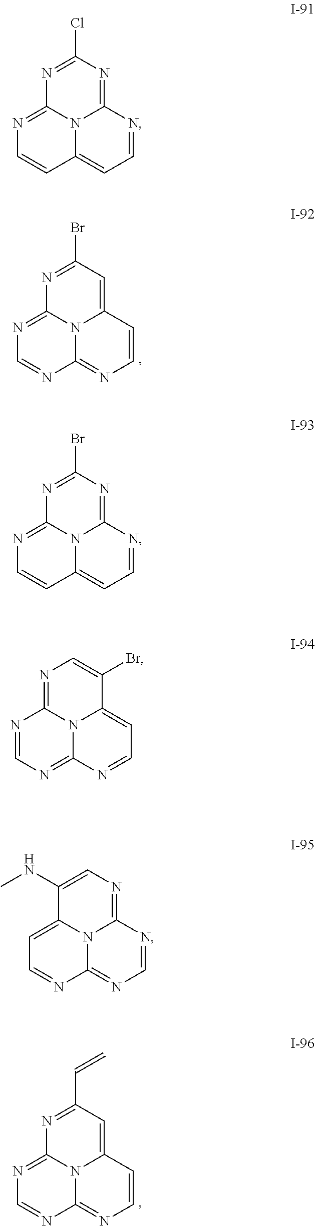

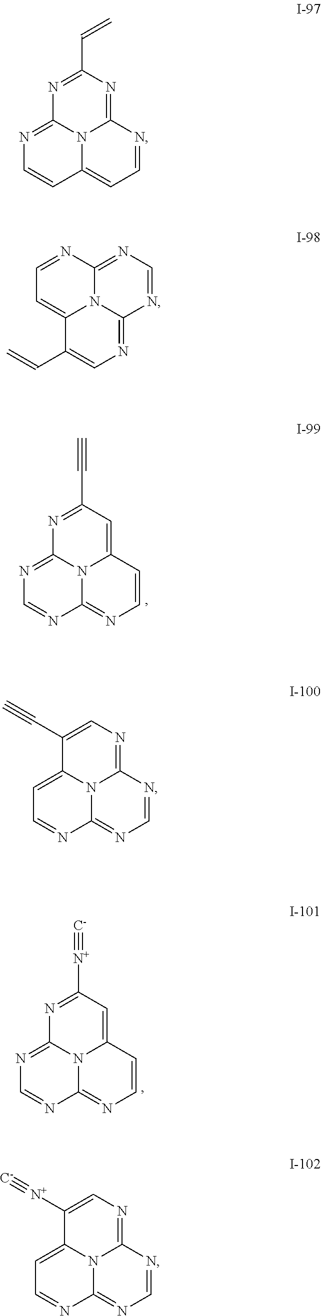

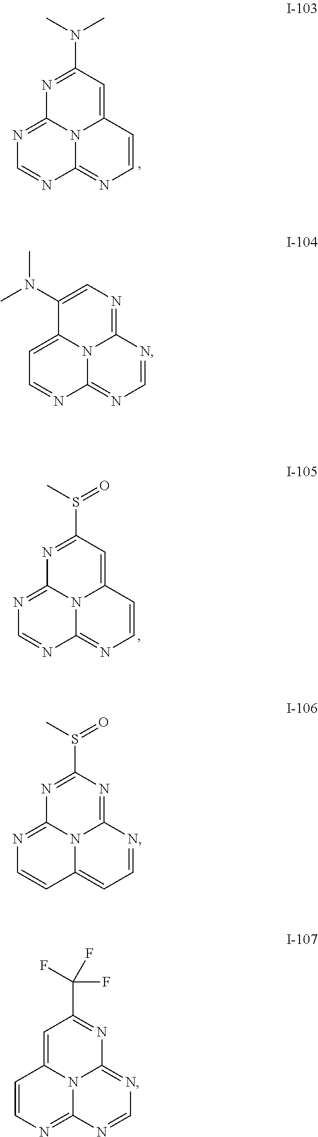 [SMILES: CN(C)C1=NC2=NC=NC3=NC=CC(=C1)N32 ; CN(C)C1=CN=C2N=CN=C3N=CC=C1N32 ; CS(=O)C1=NC2=NC=NC3=NC=CC(=C1)N32 ; CS(=O)C1=NC2=NC=CC3=CC=NC(=N1)N32 ; FC(F)(F)C1=NC2=NC=NC3=NC=CC(=C1)N32]

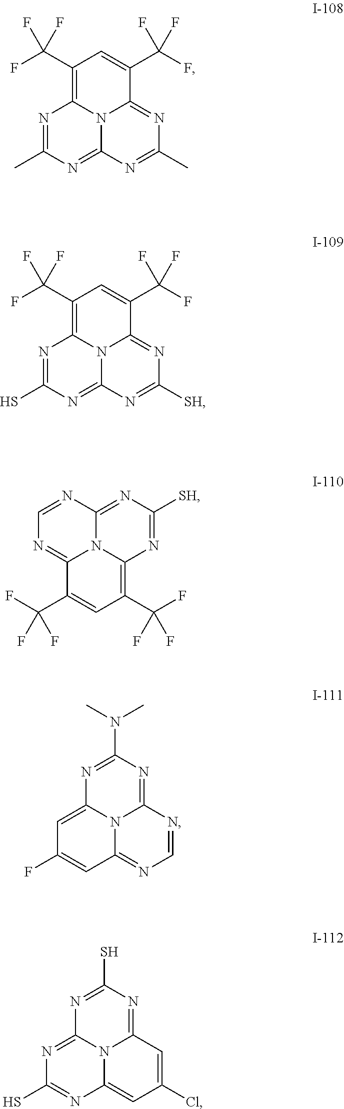 [SMILES: CC1=NC2=NC(C)=NC3=C(C(F)(F)F)C=C(C(F)(F)F)C(=N1)N23 ; FC(F)(F)C1=CC(C(F)(F)F)=C2N=C(S)N=C3N=C(S)N=C1N32 ; FC(F)(F)C1=CC(C(F)(F)F)=C2N=C(S)N=C3N=CN=C1N32 ; CN(C)C1=NC2=CC(F)=CC3=NC=NC(=N1)N23 ; SC1=NC2=CC(Cl)=CC3=NC(S)=NC(=N1)N23]

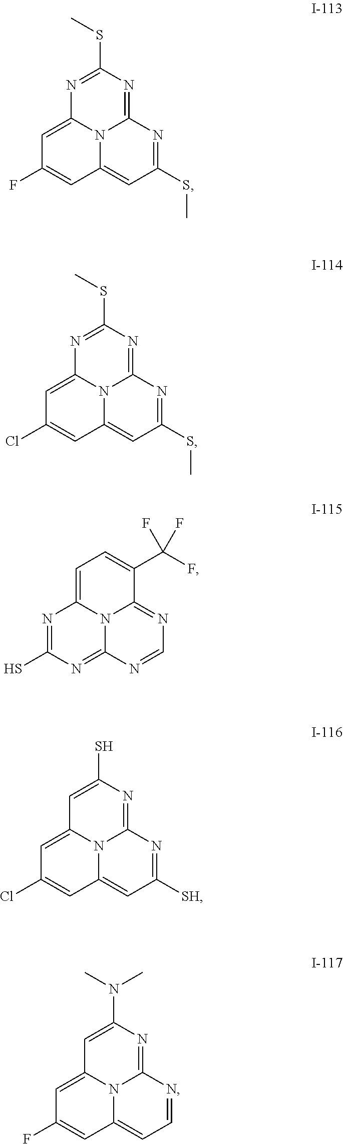 [SMILES: CSC1=NC2=NC(SC)=NC3=CC(F)=CC(=C1)N32 ; CSC1=NC2=NC(SC)=NC3=CC(Cl)=CC(=C1)N32 ; FC(F)(F)C1=CC=C2N=C(S)N=C3N=CN=C1N23 ; SC1=CC2=CC(Cl)=CC3=CC(S)=NC(=N1)N23 ; CN(C)C1=CC2=CC(F)=CC3=CC=NC(=N1)N32]

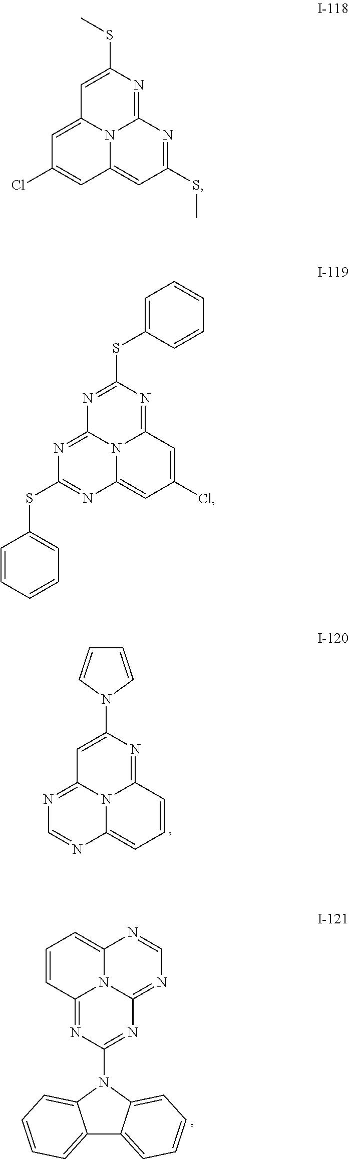 [SMILES: CSC1=CC2=CC(Cl)=CC3=CC(SC)=NC(=N1)N23 ; ClC1=CC2=NC(Sc3ccccc3)=NC3=NC(Sc4ccccc4)=NC(=C1)N23 ; C1=CC2=NC(n3cccc3)=CC3=NC=NC(=C1)N32 ; C1=CC2=NC(n3c4ccccc4c4ccccc43)=NC3=NC=NC(=C1)N23]

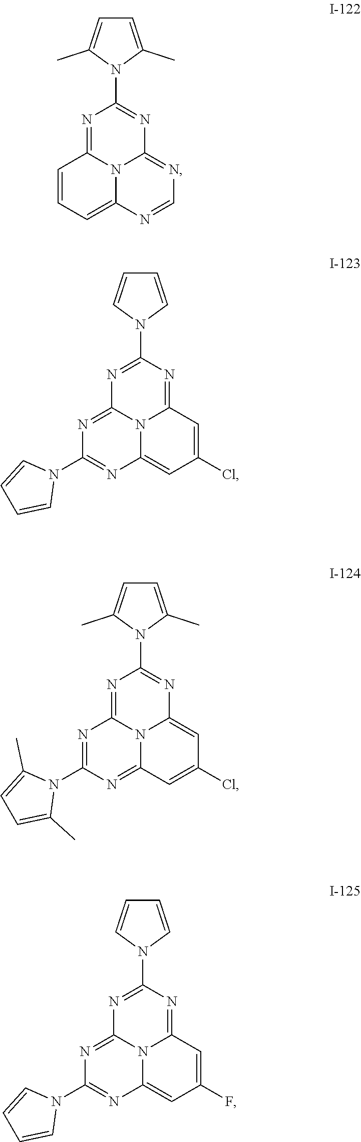

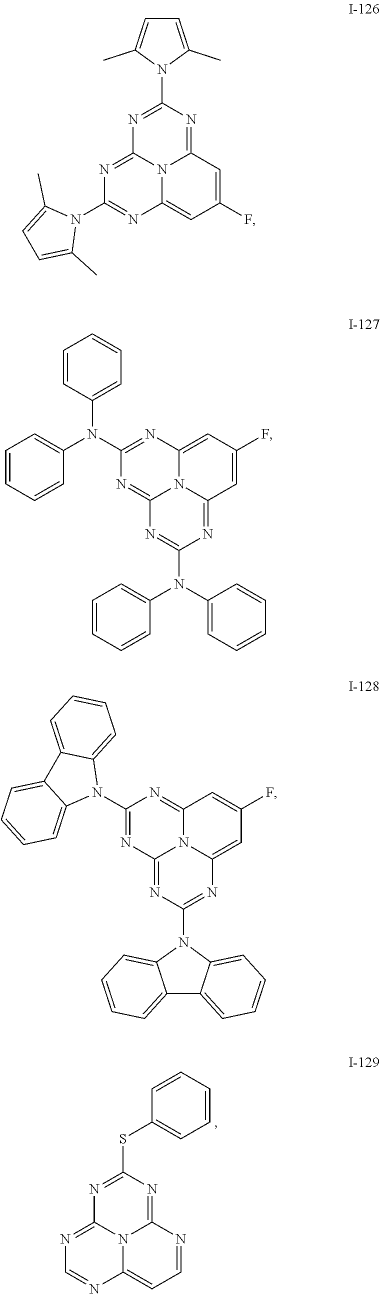 [SMILES: Cc1ccc(C)n1C1=NC2=CC(F)=CC3=NC(n4c(C)ccc4C)=NC(=N1)N23 ; FC1=CC2=NC(N(c3ccccc3)c3ccccc3)=NC3=NC(N(c4ccccc4)c4ccccc4)=NC(=C1)N23 ; FC1=CC2=NC(n3c4ccccc4c4ccccc43)=NC3=NC(n4c5ccccc5c5ccccc54)=NC(=C1)N23 ; C1=NC2=CC=NC3=NC(Sc4ccccc4)=NC(=N1)N23]

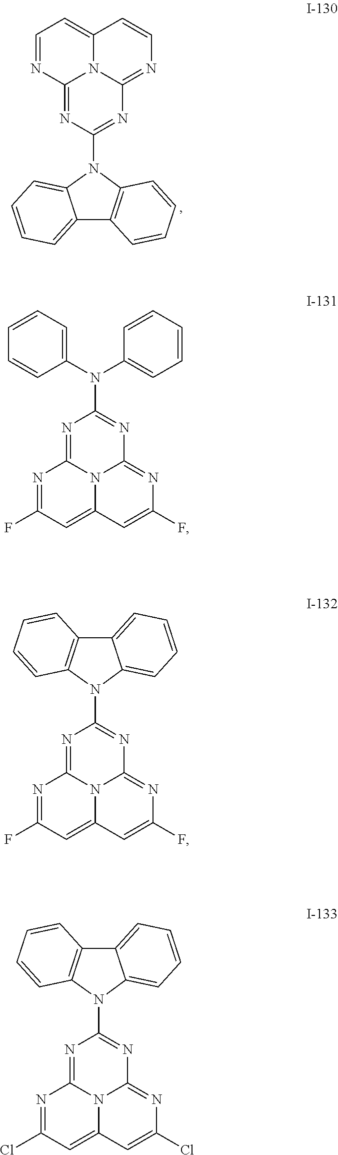 [SMILES: C1=CC2=CC=NC3=NC(n4c5ccccc5c5ccccc54)=NC(=N1)N23 ; FC1=CC2=CC(F)=NC3=NC(N(c4ccccc4)c4ccccc4)=NC(=N1)N23 ; FC1=CC2=CC(F)=NC3=NC(n4c5ccccc5c5ccccc54)=NC(=N1)N23 ; ClC1=CC2=CC(Cl)=NC3=NC(n4c5ccccc5c5ccccc54)=NC(=N1)N23]

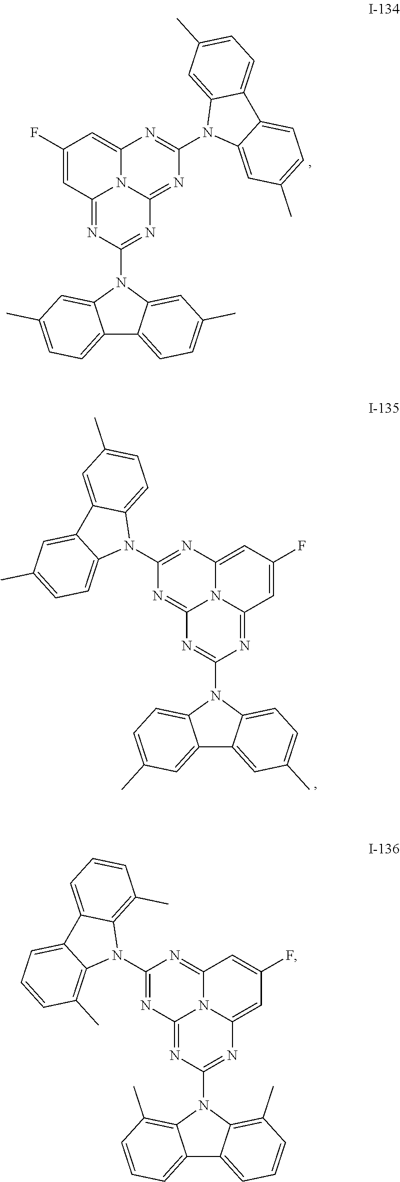

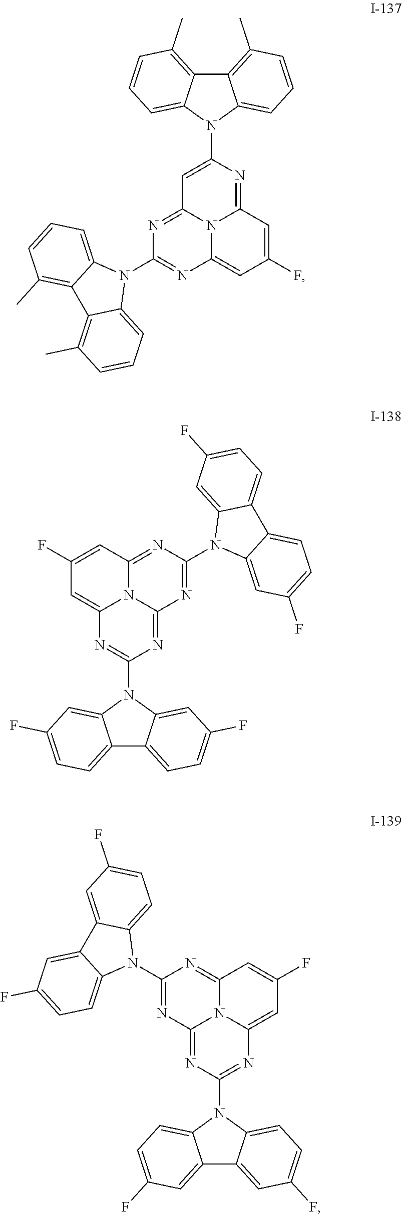 [SMILES: Cc1cccc2c1c1c(C)cccc1n2C1=CC2=NC(n3c4cccc(C)c4c4c(C)cccc43)=NC3=CC(F)=CC(=N1)N32 ; FC1=CC2=NC(n3c4cc(F)ccc4c4ccc(F)cc43)=NC3=NC(n4c5cc(F)ccc5c5ccc(F)cc54)=NC(=C1)N23 ; FC1=CC2=NC(n3c4ccc(F)cc4c4cc(F)ccc43)=NC3=NC(n4c5ccc(F)cc5c5cc(F)ccc54)=NC(=C1)N23]

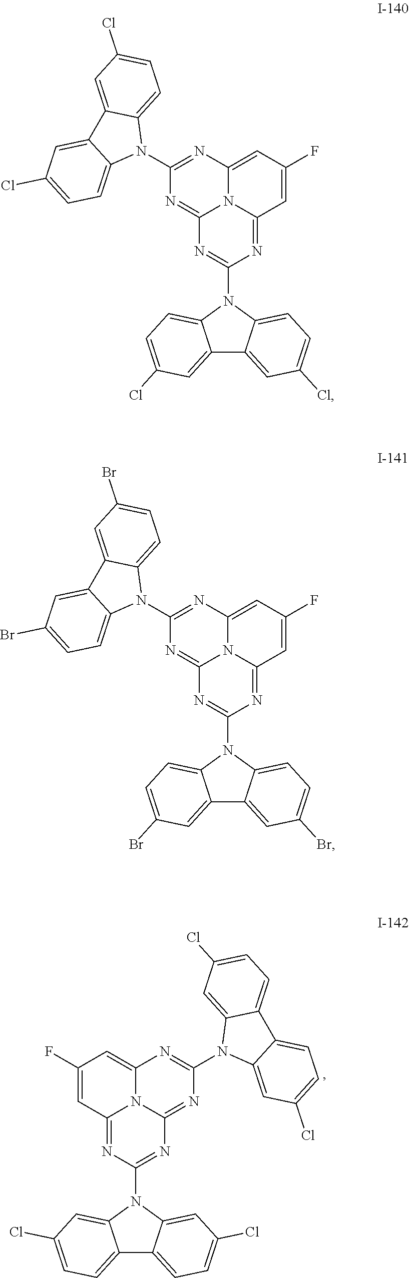 [SMILES: FC1=CC2=NC(n3c4ccc(Cl)cc4c4cc(Cl)ccc43)=NC3=NC(n4c5ccc(Cl)cc5c5cc(Cl)ccc54)=NC(=C1)N23 ; FC1=CC2=NC(n3c4ccc(Br)cc4c4cc(Br)ccc43)=NC3=NC(n4c5ccc(Br)cc5c5cc(Br)ccc54)=NC(=C1)N23 ; FC1=CC2=NC(n3c4cc(Cl)ccc4c4ccc(Cl)cc43)=NC3=NC(n4c5cc(Cl)ccc5c5ccc(Cl)cc54)=NC(=C1)N23]

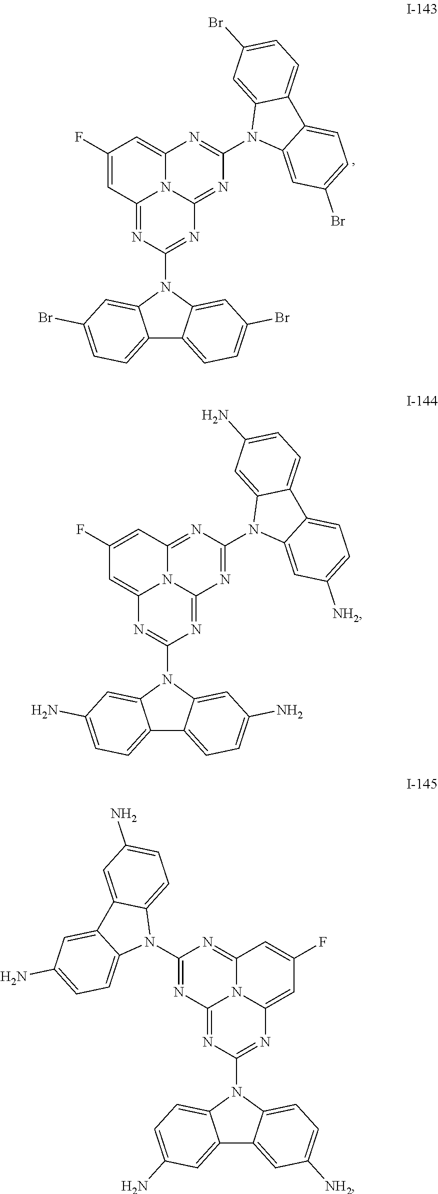 [SMILES: FC1=CC2=NC(n3c4cc(Br)ccc4c4ccc(Br)cc43)=NC3=NC(n4c5cc(Br)ccc5c5ccc(Br)cc54)=NC(=C1)N23 ; Nc1ccc2c3ccc(N)cc3n(C3=NC4=CC(F)=CC5=NC(n6c7cc(N)ccc7c7ccc(N)cc76)=NC(=N3)N45)c2c1 ; Nc1ccc2c(c1)c1cc(N)ccc1n2C1=NC2=CC(F)=CC3=NC(n4c5ccc(N)cc5c5cc(N)ccc54)=NC(=N1)N23]

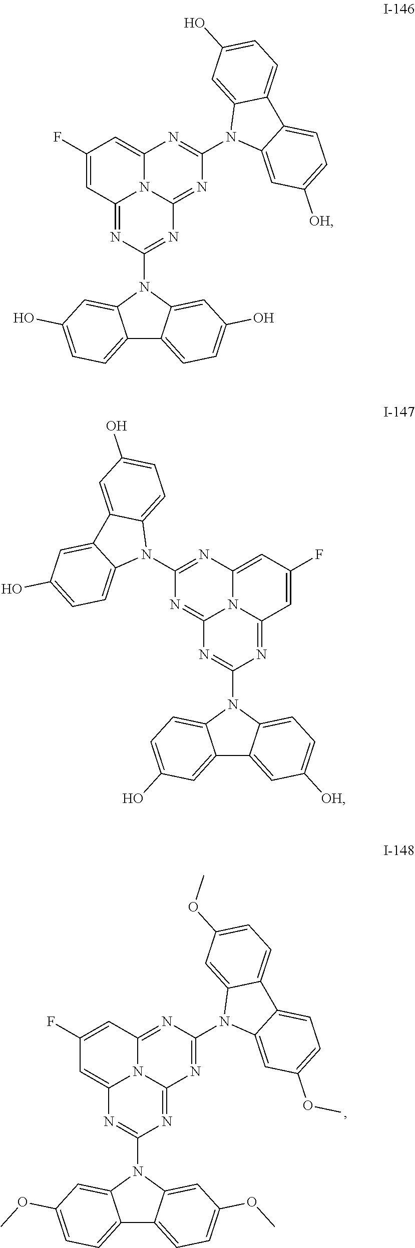

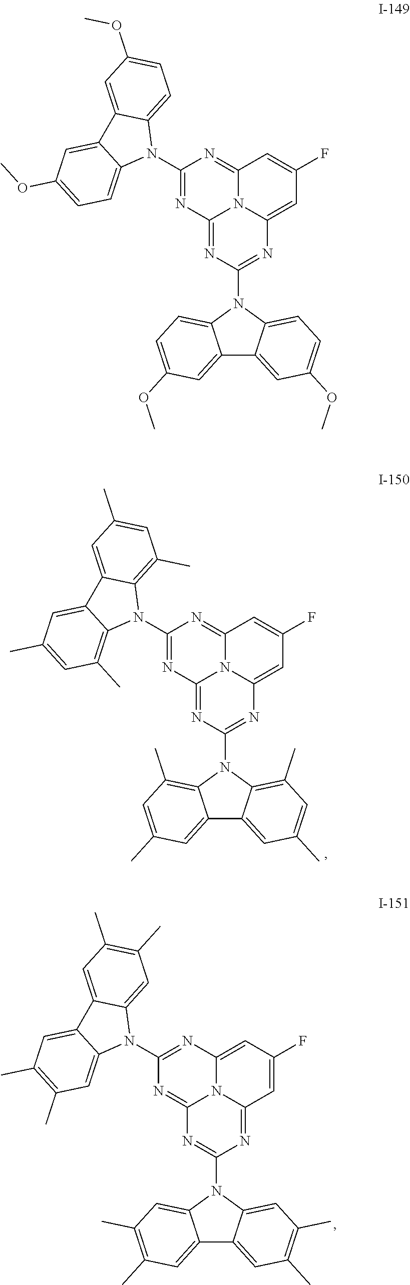

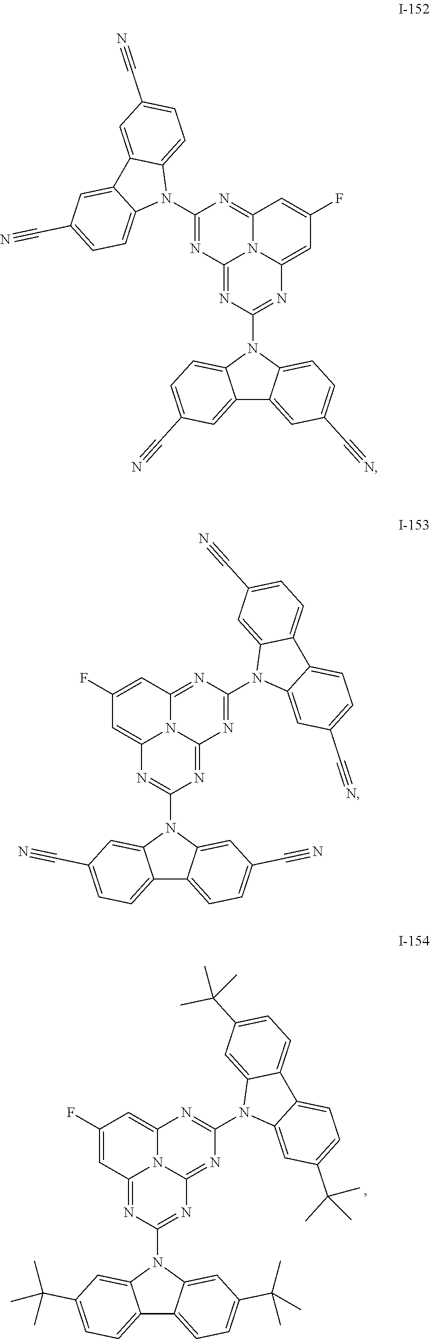 [SMILES: N#Cc1ccc2c(c1)c1cc(C#N)ccc1n2C1=NC2=CC(F)=CC3=NC(n4c5ccc(C#N)cc5c5cc(C#N)ccc54)=NC(=N1)N23 ; N#Cc1ccc2c3ccc(C#N)cc3n(C3=NC4=CC(F)=CC5=NC(n6c7cc(C#N)ccc7c7ccc(C#N)cc76)=NC(=N3)N45)c2c1 ; CC(C)(C)c1ccc2c3ccc(C(C)(C)C)cc3n(C3=NC4=CC(F)=CC5=NC(n6c7cc(C(C)(C)C)ccc7c7ccc(C(C)(C)C)cc76)=NC(=N3)N45)c2c1]

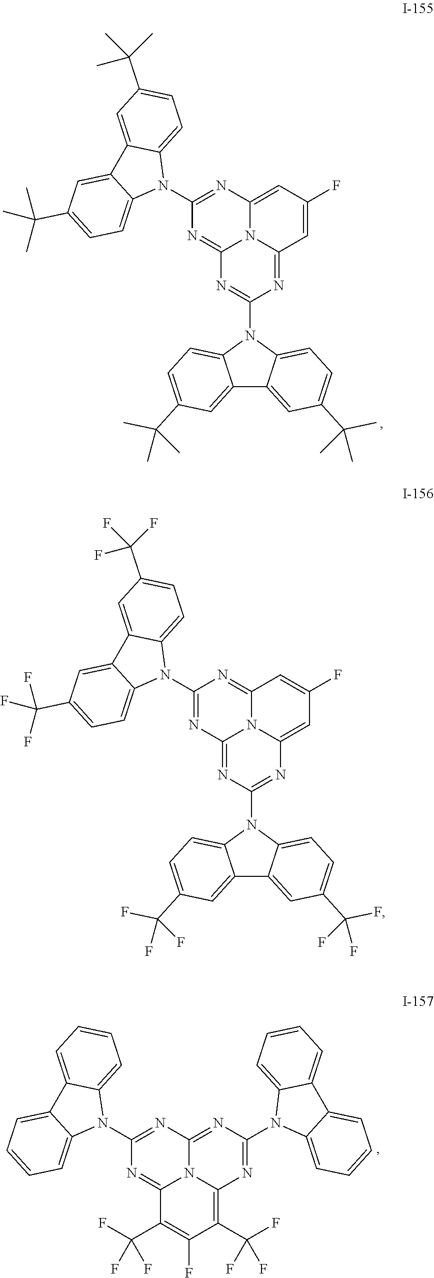 [SMILES: CC(C)(C)c1ccc2c(c1)c1cc(C(C)(C)C)ccc1n2C1=NC2=CC(F)=CC3=NC(n4c5ccc(C(C)(C)C)cc5c5cc(C(C)(C)C)ccc54)=NC(=N1)N23 ; FC1=CC2=NC(n3c4ccc(C(F)(F)F)cc4c4cc(C(F)(F)F)ccc43)=NC3=NC(n4c5ccc(C(F)(F)F)cc5c5cc(C(F)(F)F)ccc54)=NC(=C1)N23 ; FC1=C(C(F)(F)F)C2=NC(n3c4ccccc4c4ccccc43)=NC3=NC(n4c5ccccc5c5ccccc54)=NC(=C1C(F)(F)F)N32]

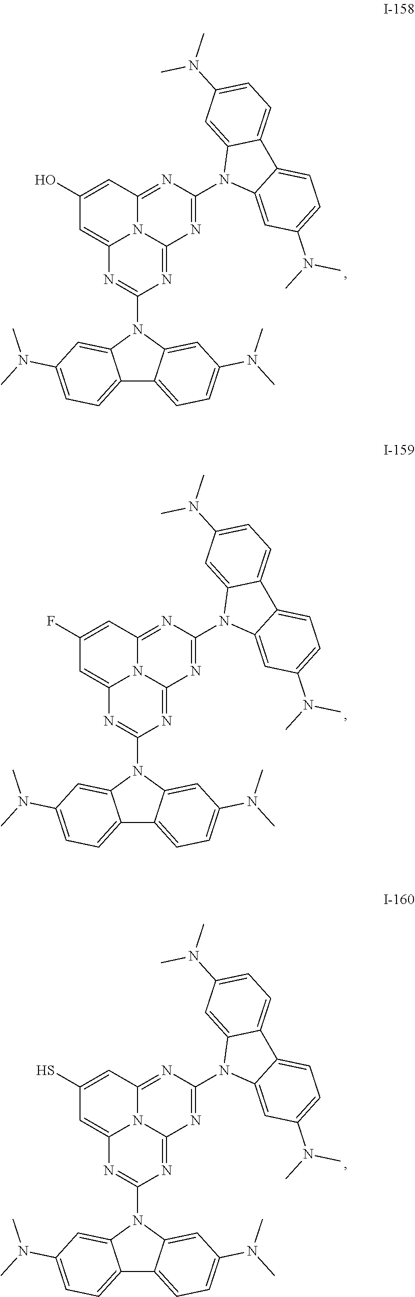

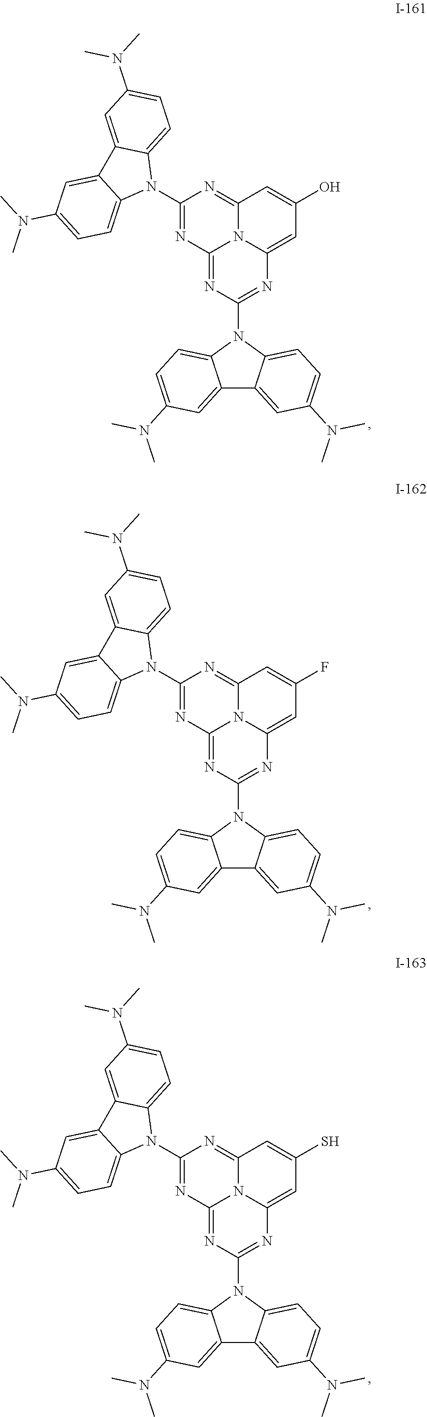

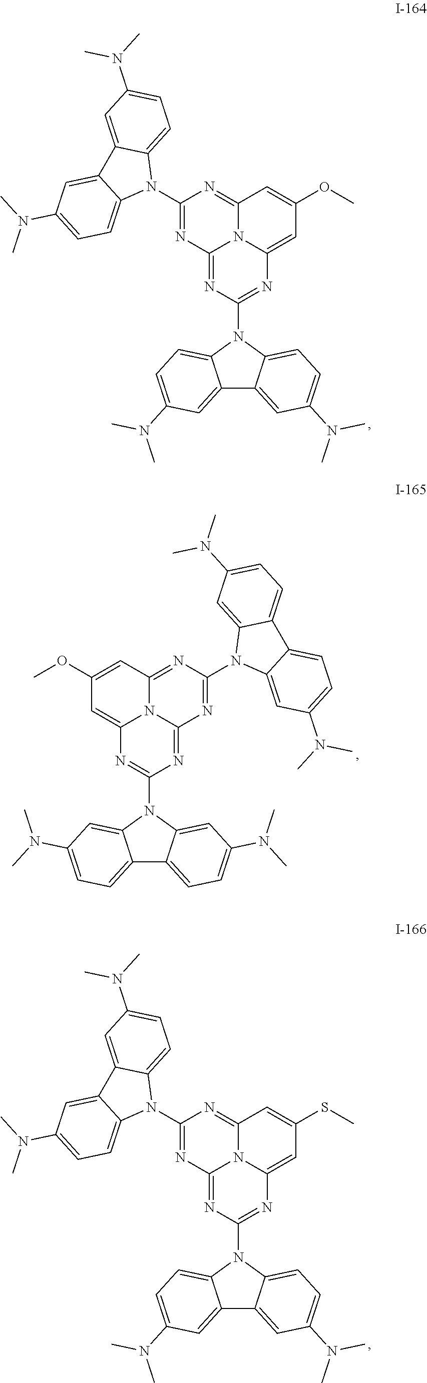

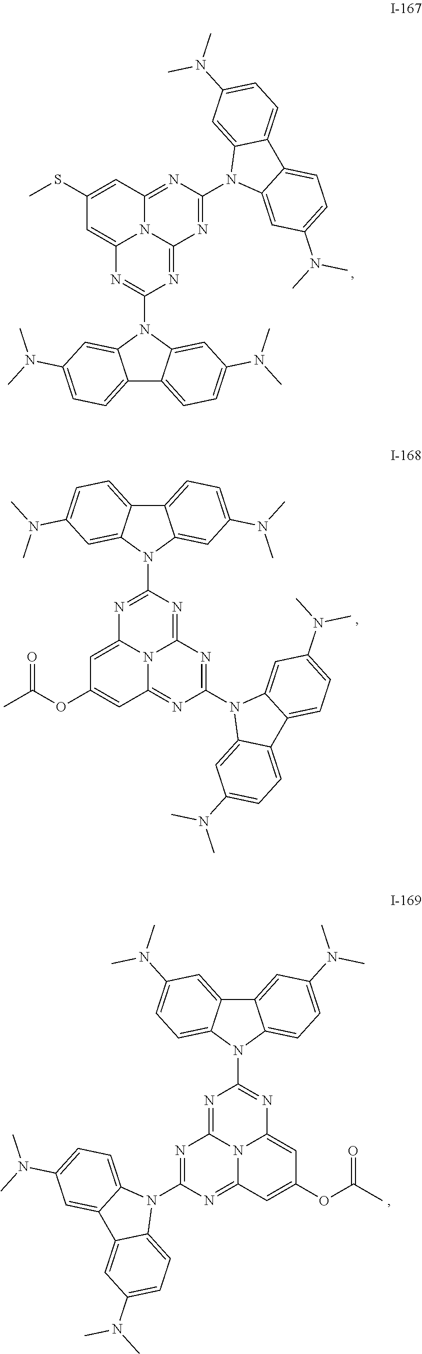

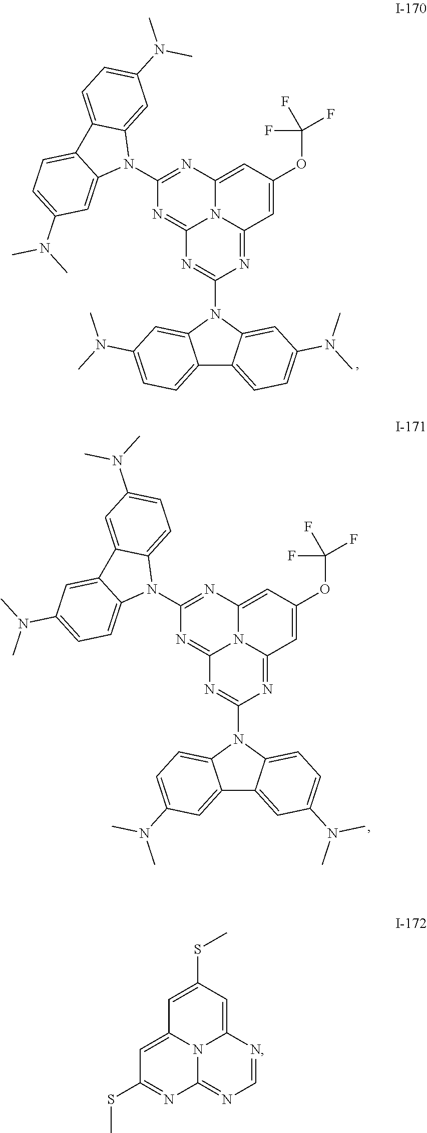

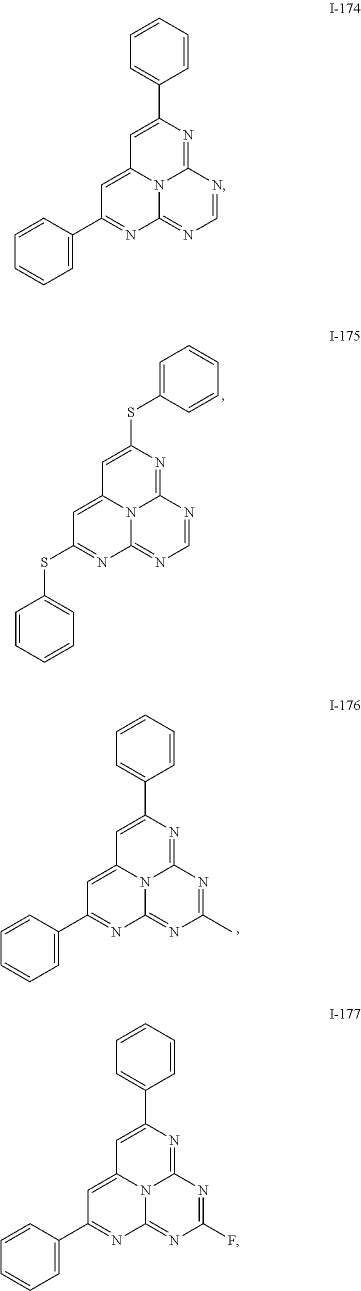 [SMILES: C1=NC2=NC(c3ccccc3)=CC3=CC(c4ccccc4)=NC(=N1)N32 ; C1=NC2=NC(Sc3ccccc3)=CC3=CC(Sc4ccccc4)=NC(=N1)N32 ; CC1=NC2=NC(c3ccccc3)=CC3=CC(c4ccccc4)=NC(=N1)N32 ; FC1=NC2=NC(c3ccccc3)=CC3=CC(c4ccccc4)=NC(=N1)N32]

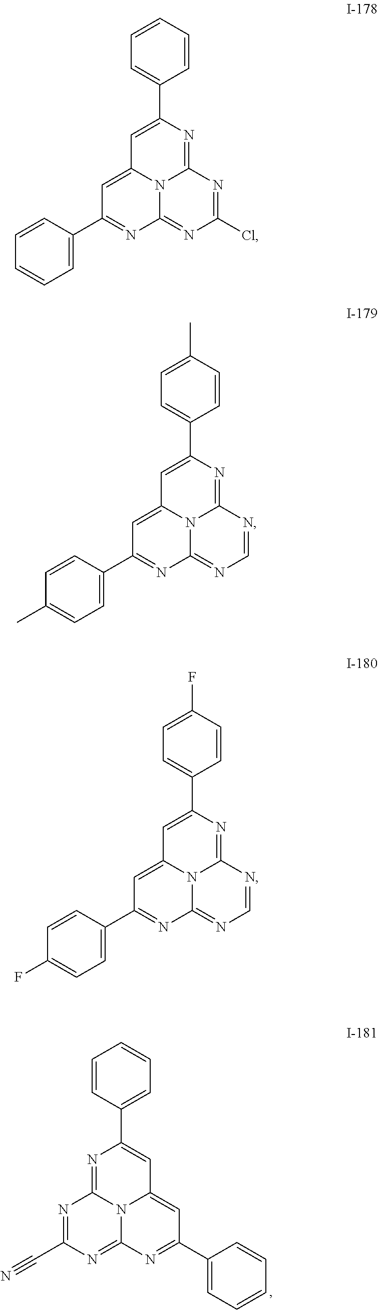 [SMILES: ClC1=NC2=NC(c3ccccc3)=CC3=CC(c4ccccc4)=NC(=N1)N32 ; Cc1ccc(C2=CC3=CC(c4ccc(C)cc4)=NC4=NC=NC(=N2)N34)cc1 ; Fc1ccc(C2=CC3=CC(c4ccc(F)cc4)=NC4=NC=NC(=N2)N34)cc1 ; N#CC1=NC2=NC(c3ccccc3)=CC3=CC(c4ccccc4)=NC(=N1)N32]

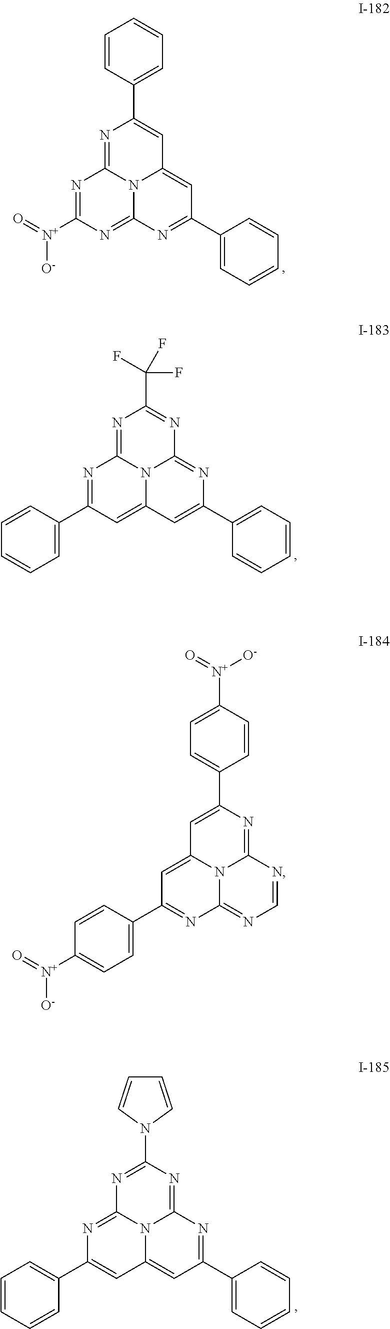 [SMILES: O=[N+]([O-])C1=NC2=NC(c3ccccc3)=CC3=CC(c4ccccc4)=NC(=N1)N32 ; FC(F)(F)C1=NC2=NC(c3ccccc3)=CC3=CC(c4ccccc4)=NC(=N1)N32 ; O=[N+]([O-])c1ccc(C2=CC3=CC(c4ccc([N+](=O)[O-])cc4)=NC4=NC=NC(=N2)N34)cc1 ; C1=C(c2ccccc2)N=C2N=C(n3cccc3)N=C3N=C(c4ccccc4)C=C1N23]

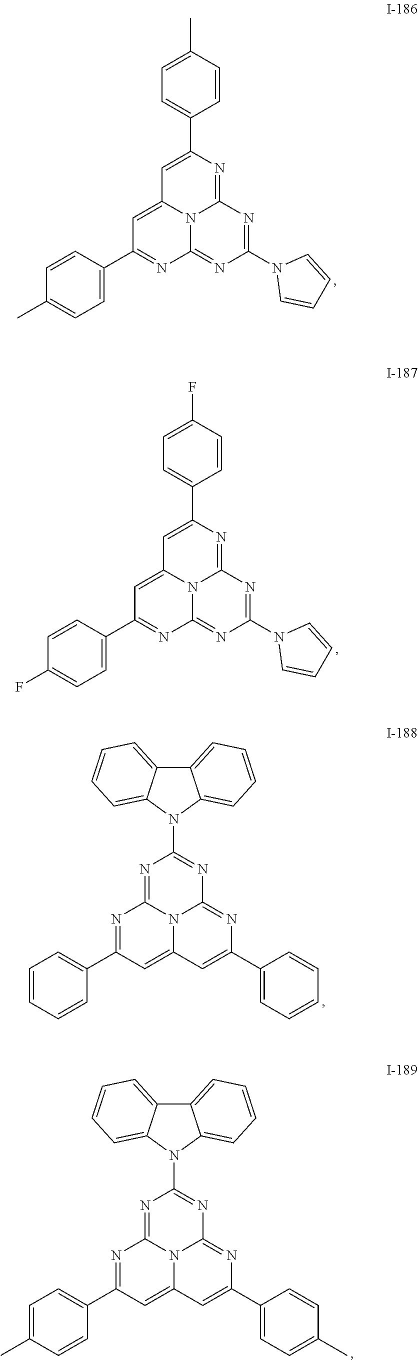

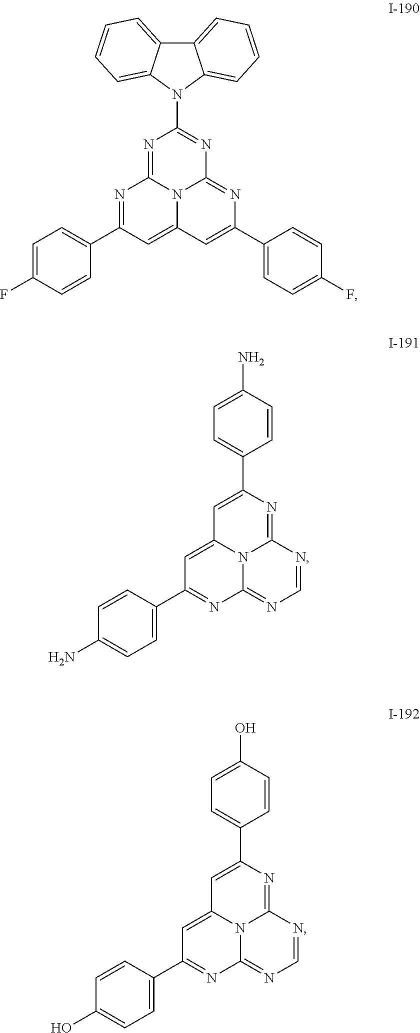 [SMILES: Fc1ccc(C2=CC3=CC(c4ccc(F)cc4)=NC4=NC(n5c6ccccc6c6ccccc65)=NC(=N2)N34)cc1 ; Nc1ccc(C2=CC3=CC(c4ccc(N)cc4)=NC4=NC=NC(=N2)N34)cc1 ; Oc1ccc(C2=CC3=CC(c4ccc(O)cc4)=NC4=NC=NC(=N2)N34)cc1]

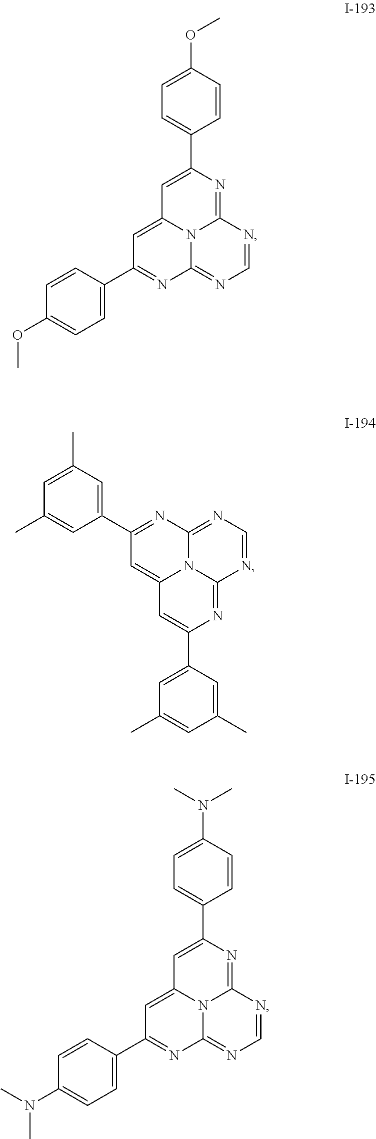

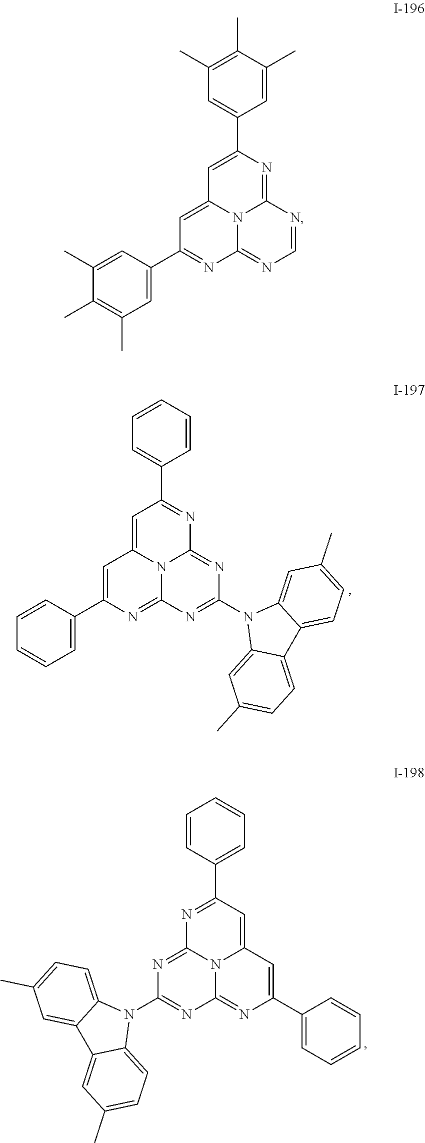

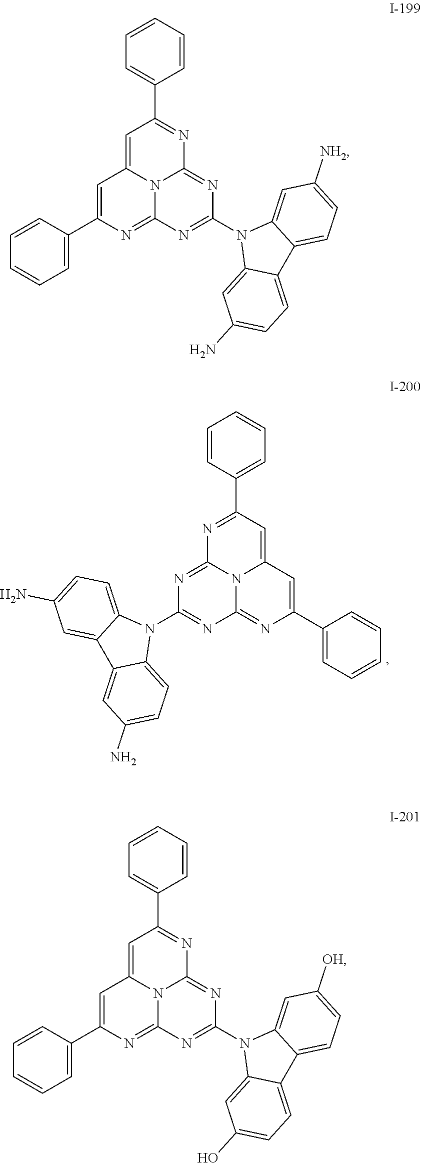 [SMILES: Nc1ccc2c3ccc(N)cc3n(C3=NC4=NC(c5ccccc5)=CC5=CC(c6ccccc6)=NC(=N3)N54)c2c1 ; Nc1ccc2c(c1)c1cc(N)ccc1n2C1=NC2=NC(c3ccccc3)=CC3=CC(c4ccccc4)=NC(=N1)N32 ; Oc1ccc2c3ccc(O)cc3n(C3=NC4=NC(c5ccccc5)=CC5=CC(c6ccccc6)=NC(=N3)N54)c2c1]

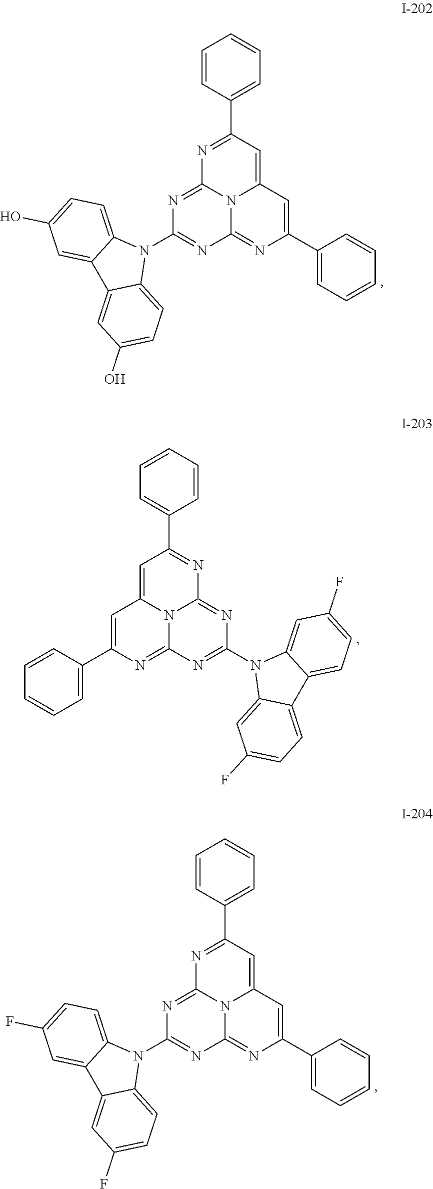 [SMILES: Oc1ccc2c(c1)c1cc(O)ccc1n2C1=NC2=NC(c3ccccc3)=CC3=CC(c4ccccc4)=NC(=N1)N32 ; Fc1ccc2c3ccc(F)cc3n(C3=NC4=NC(c5ccccc5)=CC5=CC(c6ccccc6)=NC(=N3)N54)c2c1 ; Fc1ccc2c(c1)c1cc(F)ccc1n2C1=NC2=NC(c3ccccc3)=CC3=CC(c4ccccc4)=NC(=N1)N32]

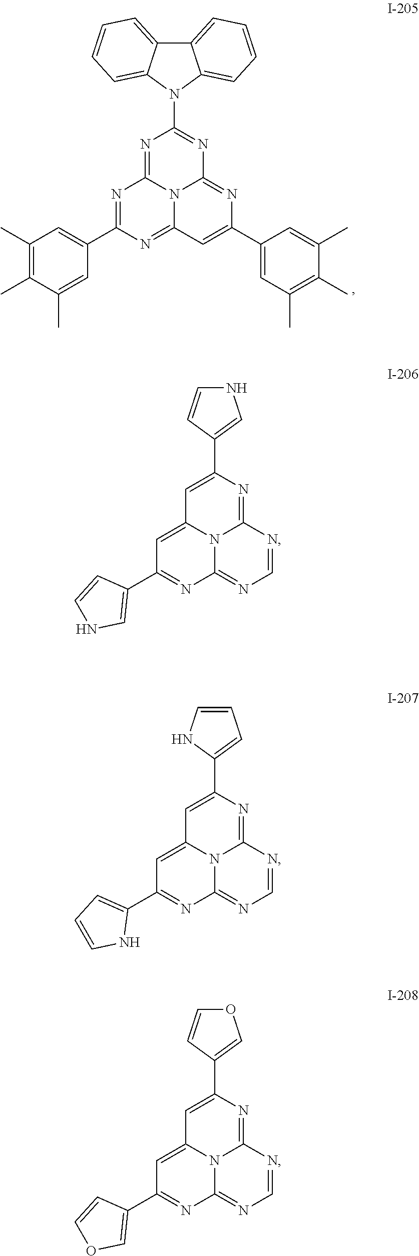

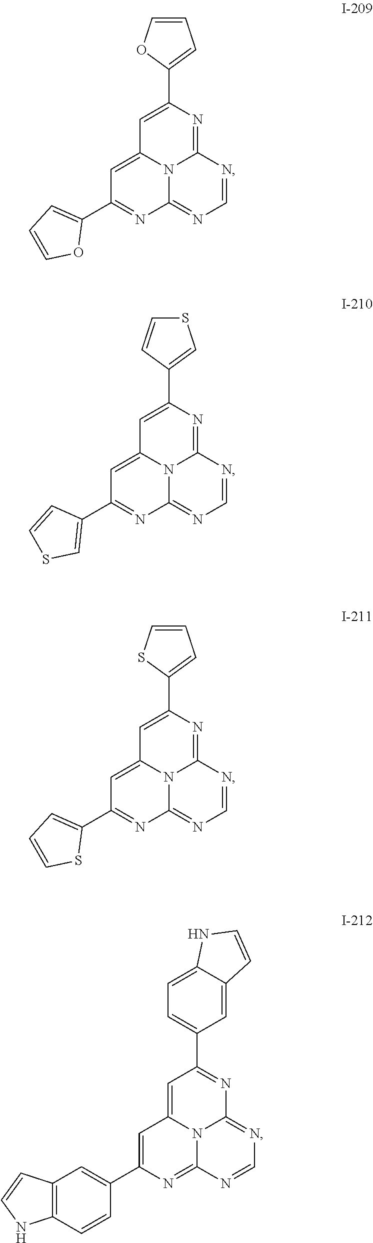 [SMILES: C1=NC2=NC(c3ccco3)=CC3=CC(c4ccco4)=NC(=N1)N32 ; C1=NC2=NC(c3ccsc3)=CC3=CC(c4ccsc4)=NC(=N1)N32 ; C1=NC2=NC(c3cccs3)=CC3=CC(c4cccs4)=NC(=N1)N32 ; C1=NC2=NC(c3ccc4[nH]ccc4c3)=CC3=CC(c4ccc5[nH]ccc5c4)=NC(=N1)N32]

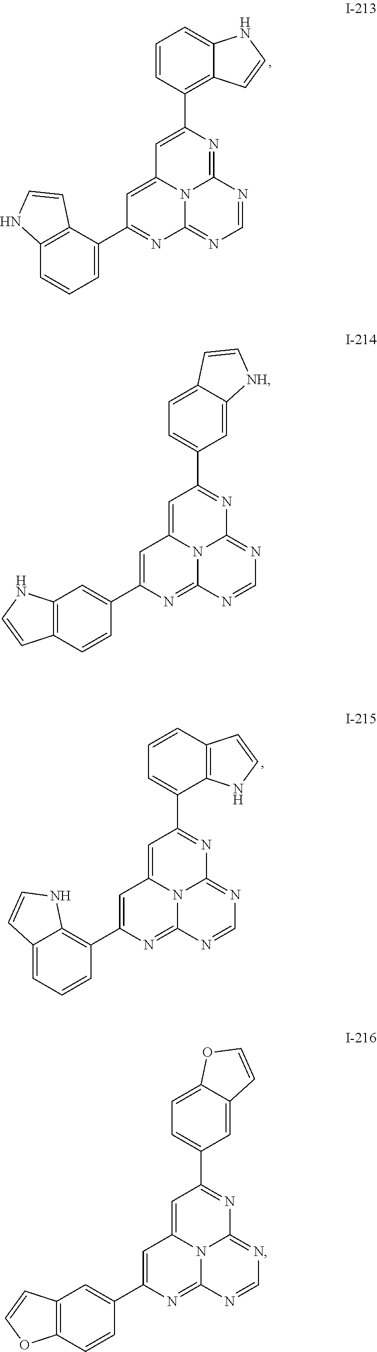 [SMILES: C1=NC2=NC(c3cccc4[nH]ccc34)=CC3=CC(c4cccc5[nH]ccc45)=NC(=N1)N32 ; C1=NC2=NC(c3ccc4cc[nH]c4c3)=CC3=CC(c4ccc5cc[nH]c5c4)=NC(=N1)N32 ; C1=NC2=NC(c3cccc4cc[nH]c34)=CC3=CC(c4cccc5cc[nH]c45)=NC(=N1)N32 ; C1=NC2=NC(c3ccc4occc4c3)=CC3=CC(c4ccc5occc5c4)=NC(=N1)N32]

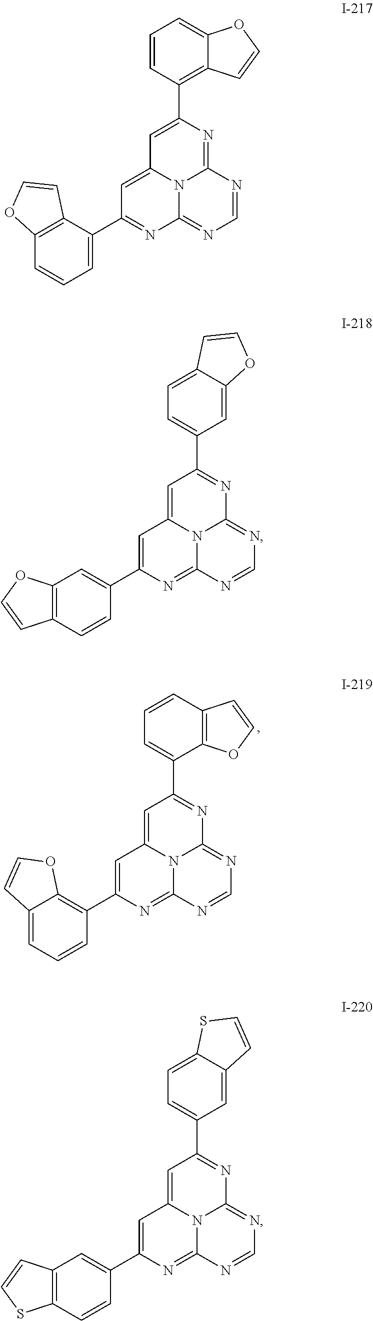 [SMILES: C1=NC2=NC(c3cccc4occc34)=CC3=CC(c4cccc5occc45)=NC(=N1)N32 ; C1=NC2=NC(c3ccc4ccoc4c3)=CC3=CC(c4ccc5ccoc5c4)=NC(=N1)N32 ; C1=NC2=NC(c3cccc4ccoc34)=CC3=CC(c4cccc5ccoc45)=NC(=N1)N32 ; C1=NC2=NC(c3ccc4sccc4c3)=CC3=CC(c4ccc5sccc5c4)=NC(=N1)N32]

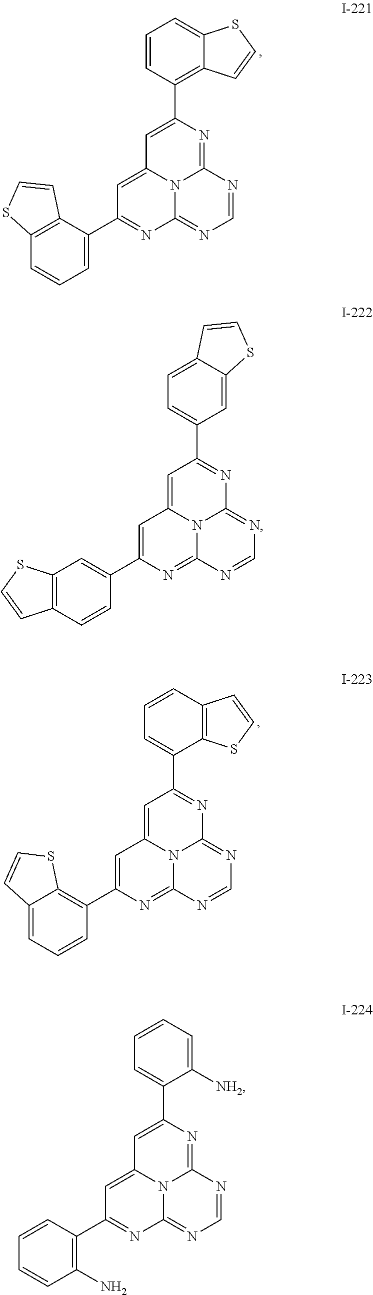 [SMILES: C1=NC2=NC(c3cccc4sccc34)=CC3=CC(c4cccc5sccc45)=NC(=N1)N32 ; C1=NC2=NC(c3ccc4ccsc4c3)=CC3=CC(c4ccc5ccsc5c4)=NC(=N1)N32 ; C1=NC2=NC(c3cccc4ccsc34)=CC3=CC(c4cccc5ccsc45)=NC(=N1)N32 ; Nc1ccccc1C1=CC2=CC(c3ccccc3N)=NC3=NC=NC(=N1)N23]

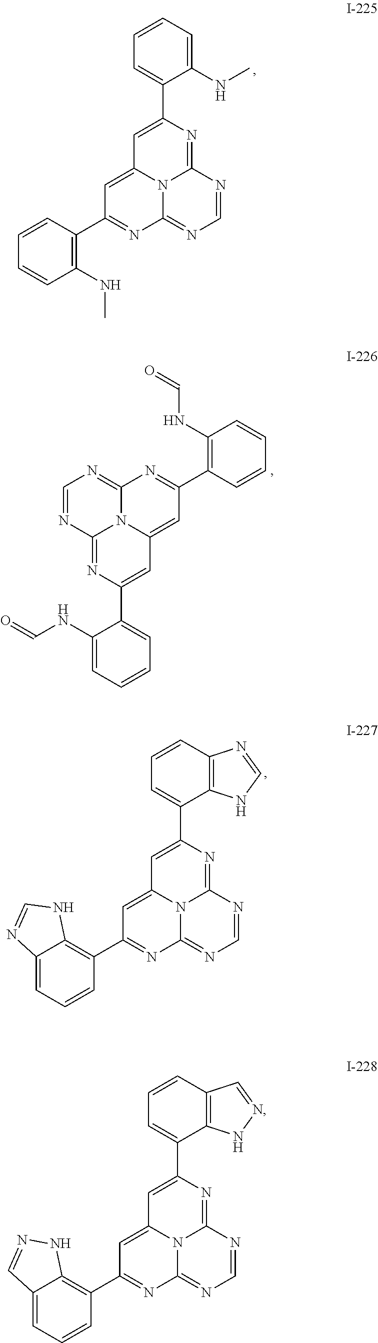 [SMILES: CNc1ccccc1C1=CC2=CC(c3ccccc3NC)=NC3=NC=NC(=N1)N23 ; O=CNc1ccccc1C1=CC2=CC(c3ccccc3NC=O)=NC3=NC=NC(=N1)N23 ; C1=NC2=NC(c3cccc4nc[nH]c34)=CC3=CC(c4cccc5nc[nH]c45)=NC(=N1)N32 ; C1=NC2=NC(c3cccc4cn[nH]c34)=CC3=CC(c4cccc5cn[nH]c45)=NC(=N1)N32]

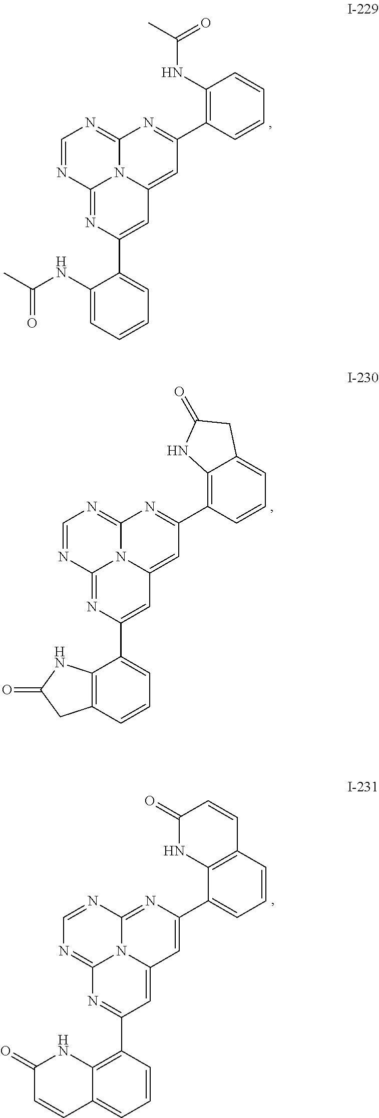 [SMILES: CC(=O)Nc1ccccc1C1=CC2=CC(c3ccccc3NC(C)=O)=NC3=NC=NC(=N1)N23 ; O=C1Cc2cccc(C3=CC4=CC(c5cccc6c5NC(=O)C6)=NC5=NC=NC(=N3)N45)c2N1 ; O=c1ccc2cccc(C3=CC4=CC(c5cccc6ccc(=O)[nH]c56)=NC5=NC=NC(=N3)N45)c2[nH]1]

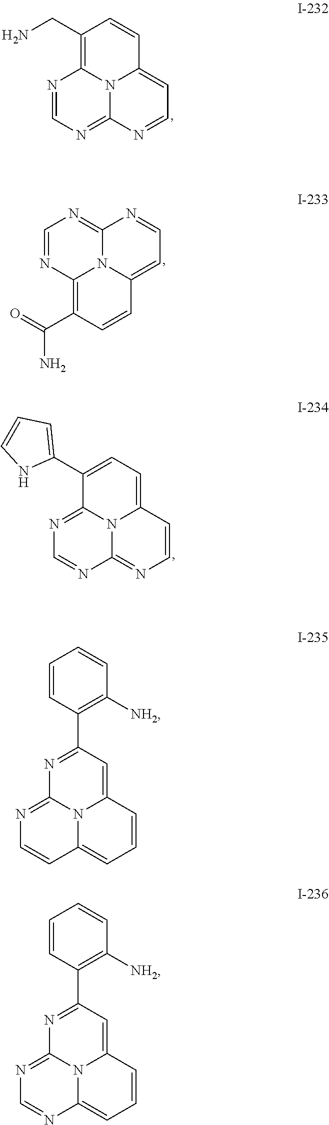 [SMILES: NCC1=C2N=CN=C3N=CC=C(C=C1)N32 ; NC(=O)C1=C2N=CN=C3N=CC=C(C=C1)N32 ; C1=CC2=CC=C(c3ccc[nH]3)C3=NC=NC(=N1)N23 ; Nc1ccccc1C1=NC2=NC=CC3=CC=CC(=C1)N32 ; Nc1ccccc1C1=NC2=NC=NC3=CC=CC(=C1)N32]

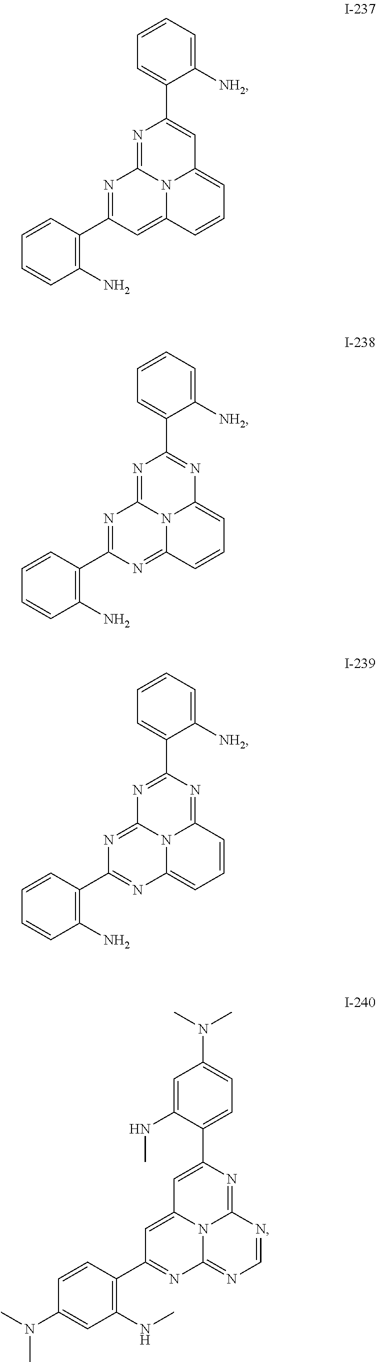 [SMILES: Nc1ccccc1C1=CC2=CC=CC3=CC(c4ccccc4N)=NC(=N1)N23 ; Nc1ccccc1C1=NC2=CC=CC3=NC(c4ccccc4N)=NC(=N1)N23 ; Nc1ccccc1C1=NC2=CC=CC3=NC(c4ccccc4N)=NC(=N1)N23 ; CNc1cc(N(C)C)ccc1C1=CC2=CC(c3ccc(N(C)C)cc3NC)=NC3=NC=NC(=N1)N23]

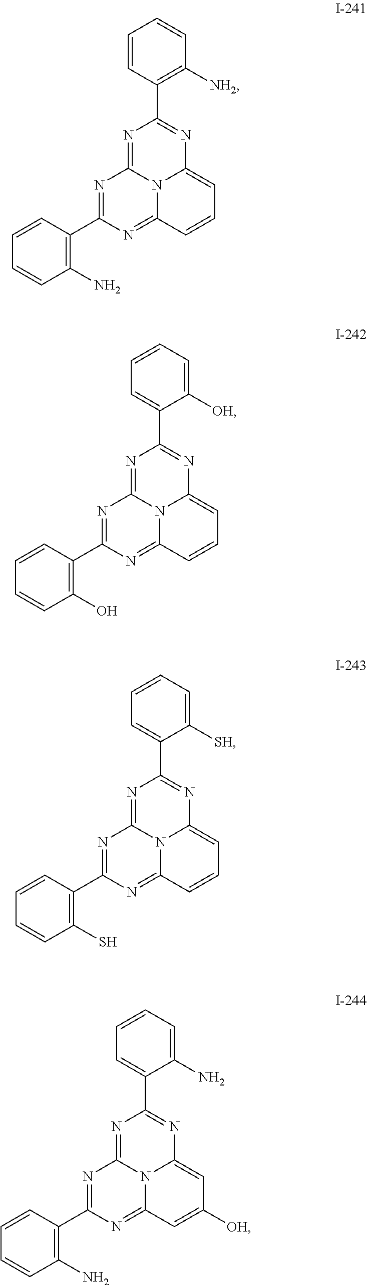 [SMILES: Nc1ccccc1C1=NC2=CC=CC3=NC(c4ccccc4N)=NC(=N1)N23 ; Oc1ccccc1C1=NC2=CC=CC3=NC(c4ccccc4O)=NC(=N1)N23 ; Sc1ccccc1C1=NC2=CC=CC3=NC(c4ccccc4S)=NC(=N1)N23 ; Nc1ccccc1C1=NC2=CC(O)=CC3=NC(c4ccccc4N)=NC(=N1)N23]

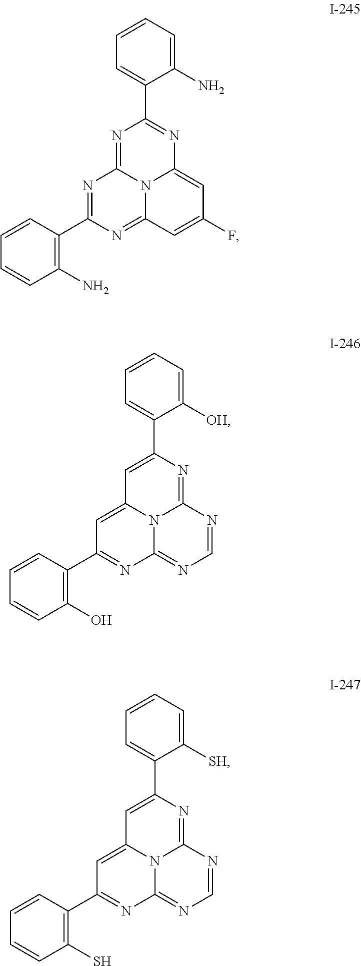 [SMILES: Nc1ccccc1C1=NC2=CC(F)=CC3=NC(c4ccccc4N)=NC(=N1)N23 ; Oc1ccccc1C1=CC2=CC(c3ccccc3O)=NC3=NC=NC(=N1)N23 ; Sc1ccccc1C1=CC2=CC(c3ccccc3S)=NC3=NC=NC(=N1)N23]

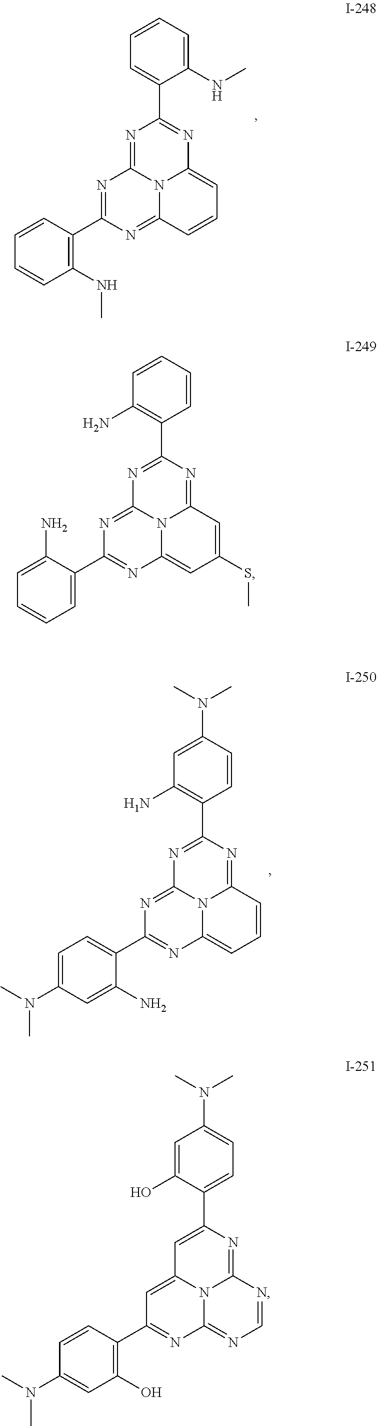

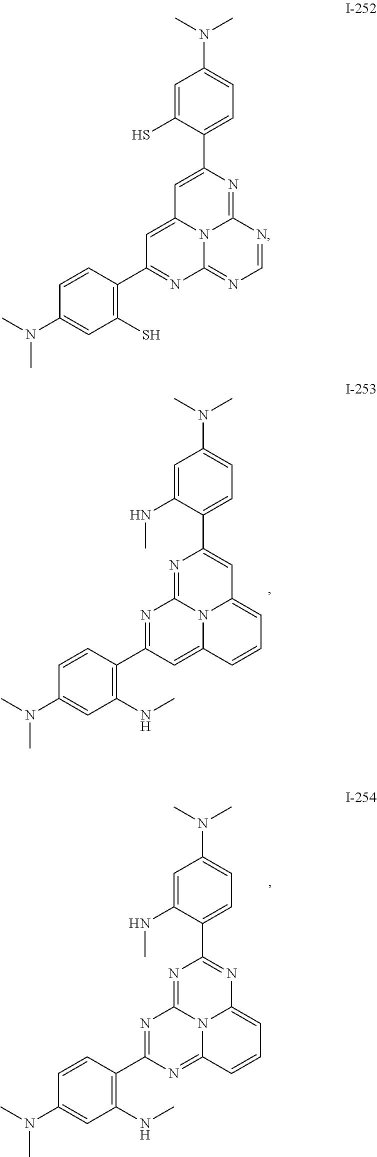 [SMILES: CN(C)c1ccc(C2=CC3=CC(c4ccc(N(C)C)cc4S)=NC4=NC=NC(=N2)N34)c(S)c1 ; CNc1cc(N(C)C)ccc1C1=CC2=CC=CC3=CC(c4ccc(N(C)C)cc4NC)=NC(=N1)N23 ; CNc1cc(N(C)C)ccc1C1=NC2=CC=CC3=NC(c4ccc(N(C)C)cc4NC)=NC(=N1)N23]

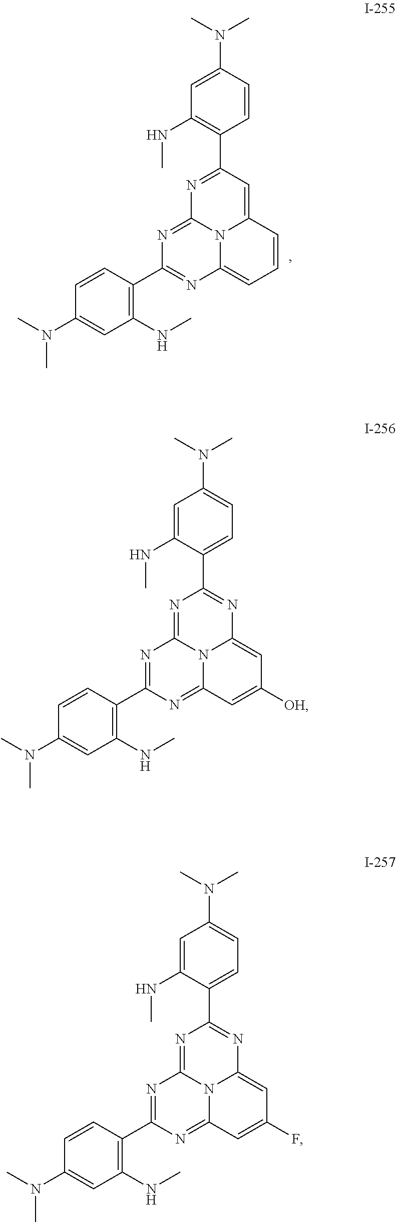 [SMILES: CNc1cc(N(C)C)ccc1C1=NC2=NC(c3ccc(N(C)C)cc3NC)=NC3=CC=CC(=C1)N32 ; CNc1cc(N(C)C)ccc1C1=NC2=CC(O)=CC3=NC(c4ccc(N(C)C)cc4NC)=NC(=N1)N23 ; CNc1cc(N(C)C)ccc1C1=NC2=CC(F)=CC3=NC(c4ccc(N(C)C)cc4NC)=NC(=N1)N23]

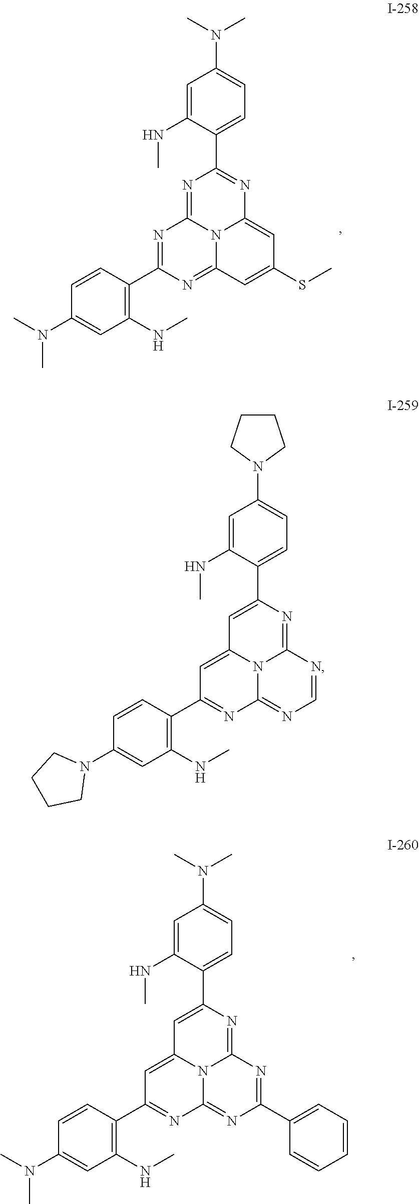

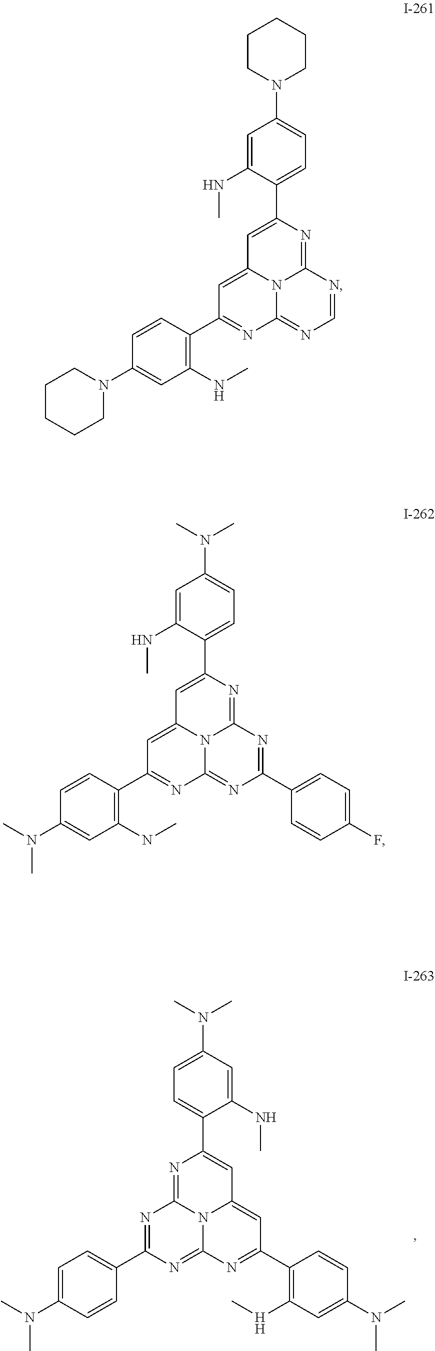

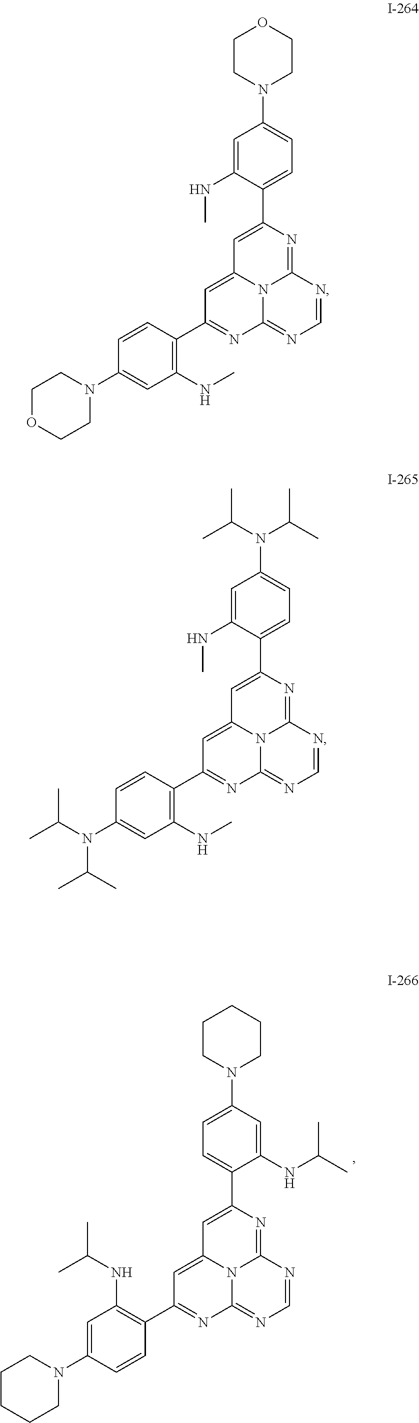 [SMILES: CNc1cc(N2CCOCC2)ccc1C1=CC2=CC(c3ccc(N4CCOCC4)cc3NC)=NC3=NC=NC(=N1)N23 ; CNc1cc(N(C(C)C)C(C)C)ccc1C1=CC2=CC(c3ccc(N(C(C)C)C(C)C)cc3NC)=NC3=NC=NC(=N1)N23 ; CC(C)Nc1cc(N2CCCCC2)ccc1C1=CC2=CC(c3ccc(N4CCCCC4)cc3NC(C)C)=NC3=NC=NC(=N1)N23]

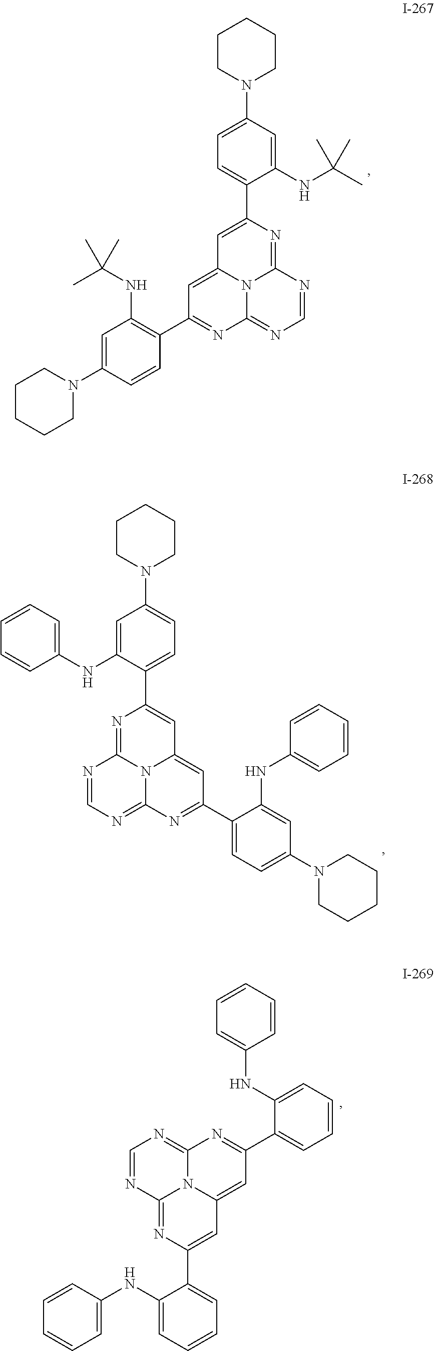 [SMILES: CC(C)(C)Nc1cc(N2CCCCC2)ccc1C1=CC2=CC(c3ccc(N4CCCCC4)cc3NC(C)(C)C)=NC3=NC=NC(=N1)N23 ; C1=NC2=NC(c3ccc(N4CCCCC4)cc3Nc3ccccc3)=CC3=CC(c4ccc(N5CCCCC5)cc4Nc4ccccc4)=NC(=N1)N32 ; C1=NC2=NC(c3ccccc3Nc3ccccc3)=CC3=CC(c4ccccc4Nc4ccccc4)=NC(=N1)N32]

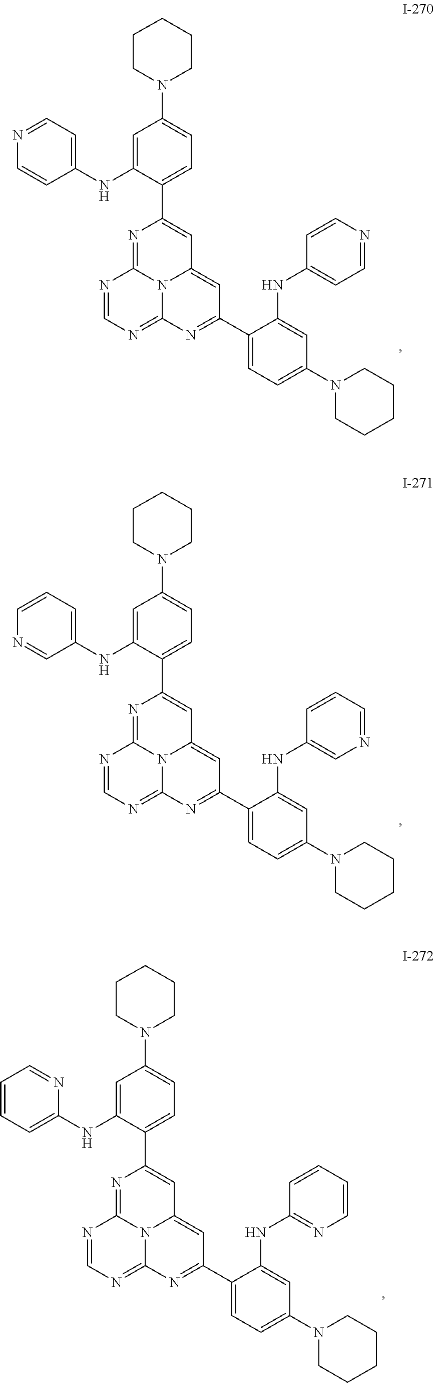 [SMILES: C1=NC2=NC(c3ccc(N4CCCCC4)cc3Nc3ccncc3)=CC3=CC(c4ccc(N5CCCCC5)cc4Nc4ccncc4)=NC(=N1)N32 ; C1=NC2=NC(c3ccc(N4CCCCC4)cc3Nc3cccnc3)=CC3=CC(c4ccc(N5CCCCC5)cc4Nc4cccnc4)=NC(=N1)N32 ; C1=NC2=NC(c3ccc(N4CCCCC4)cc3Nc3ccccn3)=CC3=CC(c4ccc(N5CCCCC5)cc4Nc4ccccn4)=NC(=N1)N32]

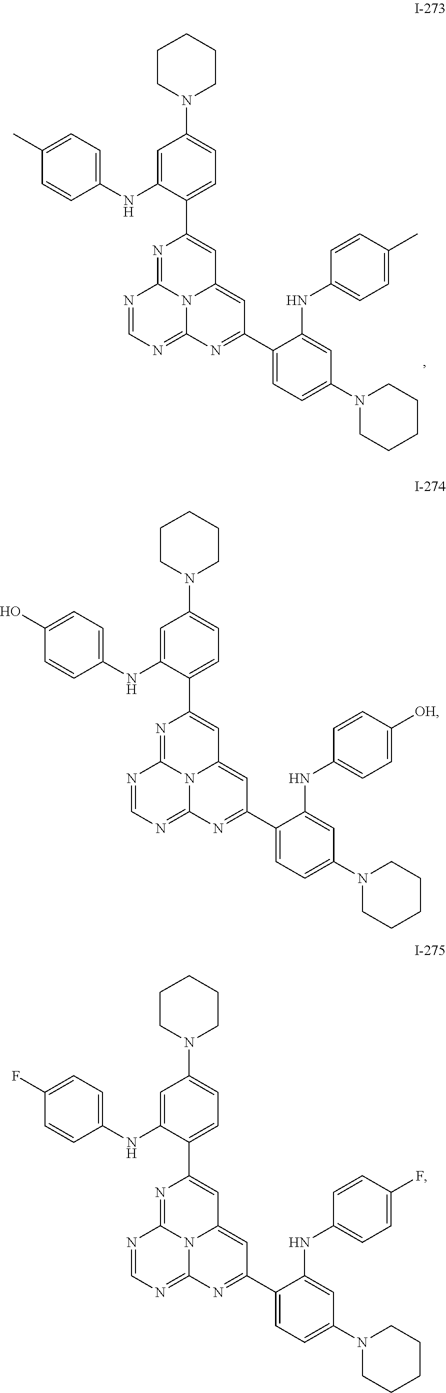

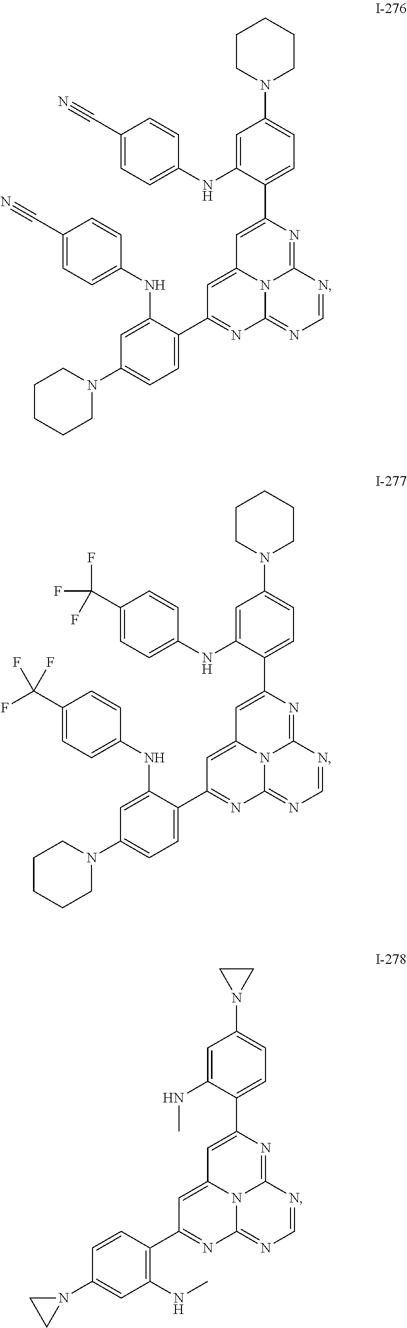 [SMILES: N#Cc1ccc(Nc2cc(N3CCCCC3)ccc2C2=CC3=CC(c4ccc(N5CCCCC5)cc4Nc4ccc(C#N)cc4)=NC4=NC=NC(=N2)N34)cc1 ; FC(F)(F)c1ccc(Nc2cc(N3CCCCC3)ccc2C2=CC3=CC(c4ccc(N5CCCCC5)cc4Nc4ccc(C(F)(F)F)cc4)=NC4=NC=NC(=N2)N34)cc1 ; CNc1cc(N2CC2)ccc1C1=CC2=CC(c3ccc(N4CC4)cc3NC)=NC3=NC=NC(=N1)N23]

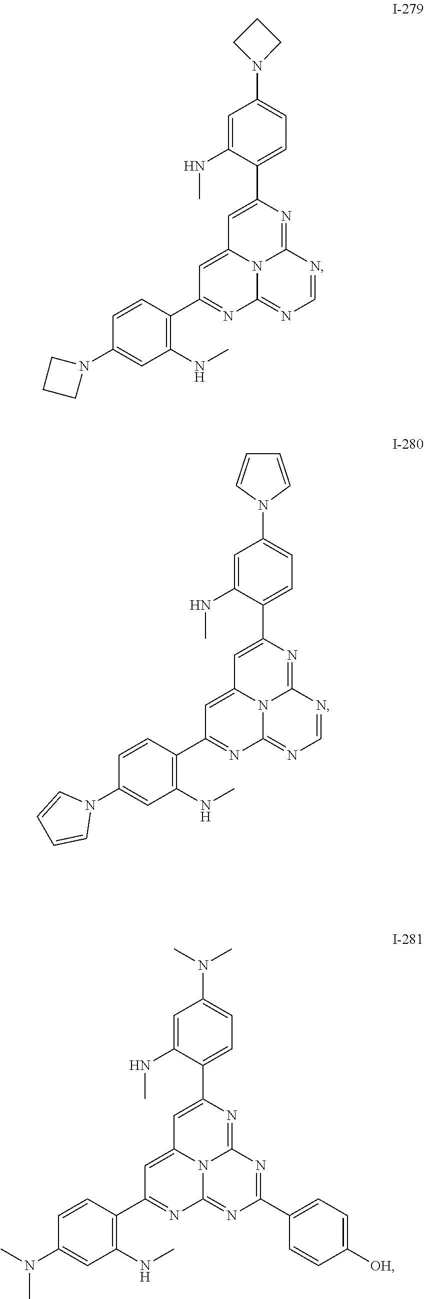 [SMILES: CNc1cc(N2CCC2)ccc1C1=CC2=CC(c3ccc(N4CCC4)cc3NC)=NC3=NC=NC(=N1)N23 ; CNc1cc(-n2cccc2)ccc1C1=CC2=CC(c3ccc(-n4cccc4)cc3NC)=NC3=NC=NC(=N1)N23 ; CNc1cc(N(C)C)ccc1C1=CC2=CC(c3ccc(N(C)C)cc3NC)=NC3=NC(c4ccc(O)cc4)=NC(=N1)N23]

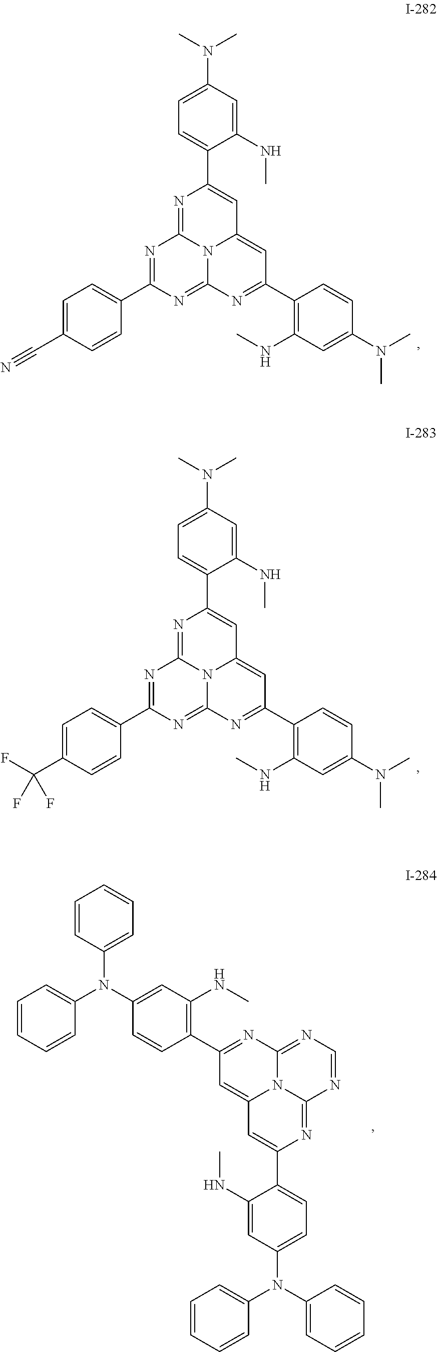 [SMILES: CNc1cc(N(C)C)ccc1C1=CC2=CC(c3ccc(N(C)C)cc3NC)=NC3=NC(c4ccc(C#N)cc4)=NC(=N1)N23 ; CNc1cc(N(C)C)ccc1C1=CC2=CC(c3ccc(N(C)C)cc3NC)=NC3=NC(c4ccc(C(F)(F)F)cc4)=NC(=N1)N23 ; CNc1cc(N(c2ccccc2)c2ccccc2)ccc1C1=CC2=CC(c3ccc(N(c4ccccc4)c4ccccc4)cc3NC)=NC3=NC=NC(=N1)N23]

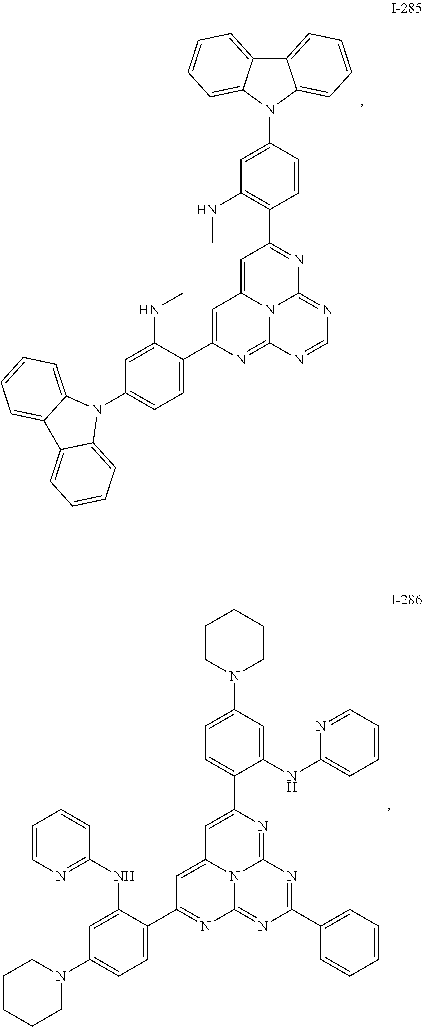 [SMILES: CNc1cc(-n2c3ccccc3c3ccccc32)ccc1C1=CC2=CC(c3ccc(-n4c5ccccc5c5ccccc54)cc3NC)=NC3=NC=NC(=N1)N23 ; C1=C(c2ccc(N3CCCCC3)cc2Nc2ccccn2)N=C2N=C(c3ccccc3)N=C3N=C(c4ccc(N5CCCCC5)cc4Nc4ccccn4)C=C1N23]

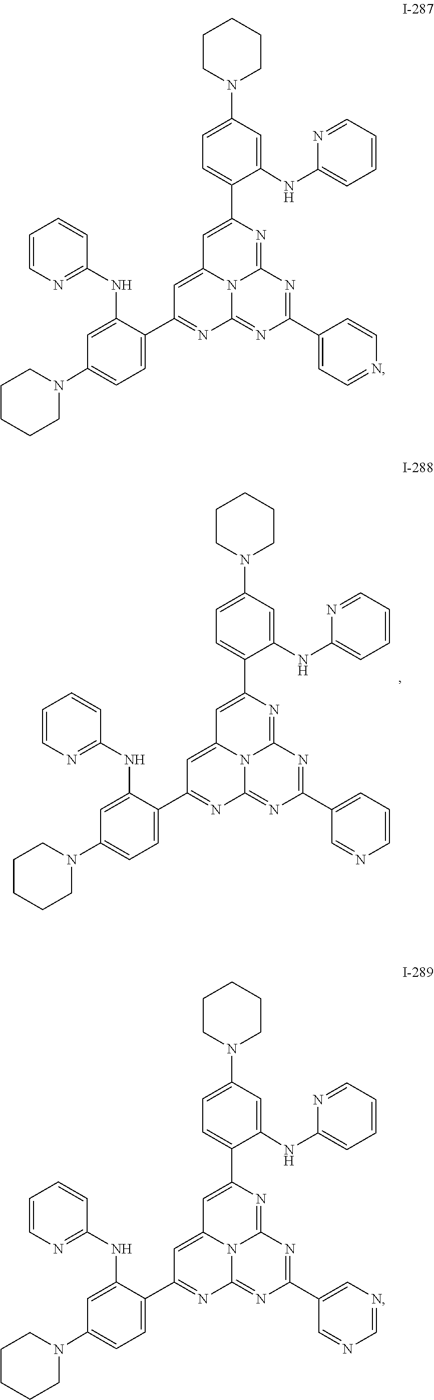 [SMILES: C1=C(c2ccc(N3CCCCC3)cc2Nc2ccccn2)N=C2N=C(c3ccncc3)N=C3N=C(c4ccc(N5CCCCC5)cc4Nc4ccccn4)C=C1N23 ; C1=C(c2ccc(N3CCCCC3)cc2Nc2ccccn2)N=C2N=C(c3cccnc3)N=C3N=C(c4ccc(N5CCCCC5)cc4Nc4ccccn4)C=C1N23 ; C1=C(c2ccc(N3CCCCC3)cc2Nc2ccccn2)N=C2N=C(c3cncnc3)N=C3N=C(c4ccc(N5CCCCC5)cc4Nc4ccccn4)C=C1N23]

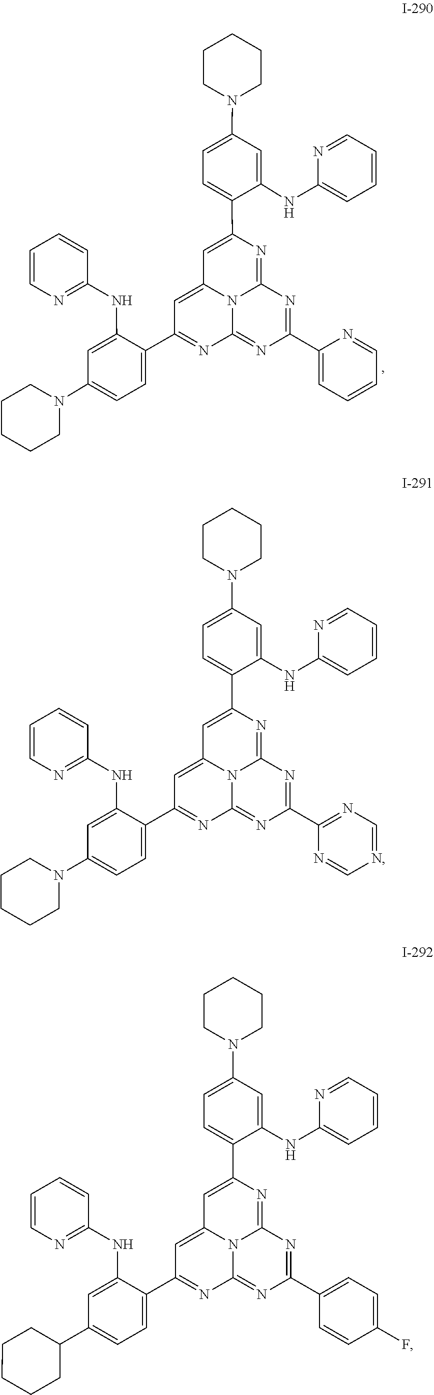 [SMILES: C1=C(c2ccc(N3CCCCC3)cc2Nc2ccccn2)N=C2N=C(c3ccccn3)N=C3N=C(c4ccc(N5CCCCC5)cc4Nc4ccccn4)C=C1N23 ; C1=C(c2ccc(N3CCCCC3)cc2Nc2ccccn2)N=C2N=C(c3ncncn3)N=C3N=C(c4ccc(N5CCCCC5)cc4Nc4ccccn4)C=C1N23 ; Fc1ccc(C2=NC3=NC(c4ccc(N5CCCCC5)cc4Nc4ccccn4)=CC4=CC(c5ccc(C6CCCCC6)cc5Nc5ccccn5)=NC(=N2)N43)cc1]

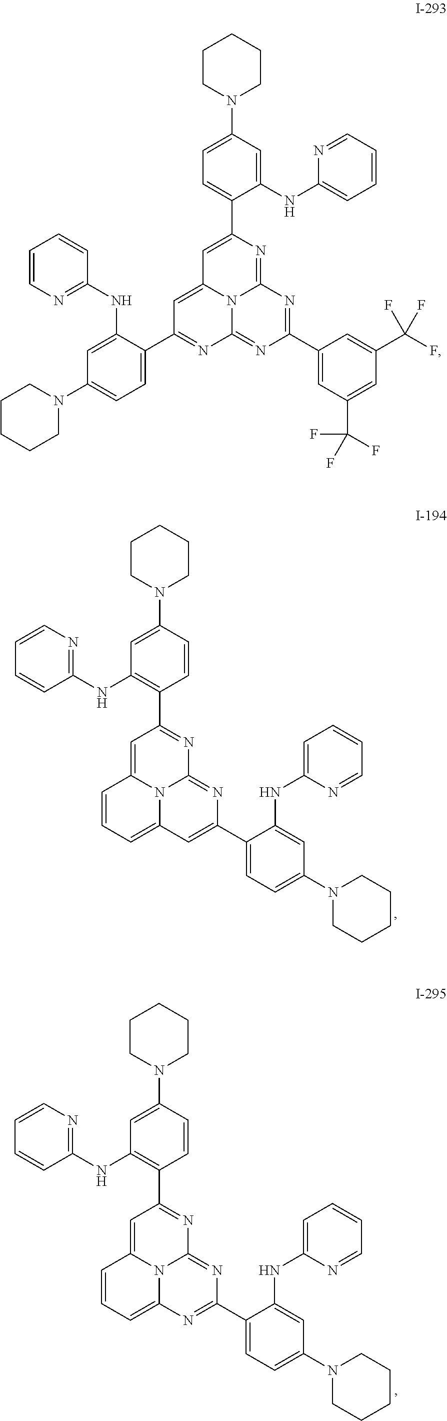 [SMILES: FC(F)(F)c1cc(C2=NC3=NC(c4ccc(N5CCCCC5)cc4Nc4ccccn4)=CC4=CC(c5ccc(N6CCCCC6)cc5Nc5ccccn5)=NC(=N2)N43)cc(C(F)(F)F)c1 ; C1=CC2=CC(c3ccc(N4CCCCC4)cc3Nc3ccccn3)=NC3=NC(c4ccc(N5CCCCC5)cc4Nc4ccccn4)=CC(=C1)N23 ; C1=CC2=CC(c3ccc(N4CCCCC4)cc3Nc3ccccn3)=NC3=NC(c4ccc(N5CCCCC5)cc4Nc4ccccn4)=NC(=C1)N23]

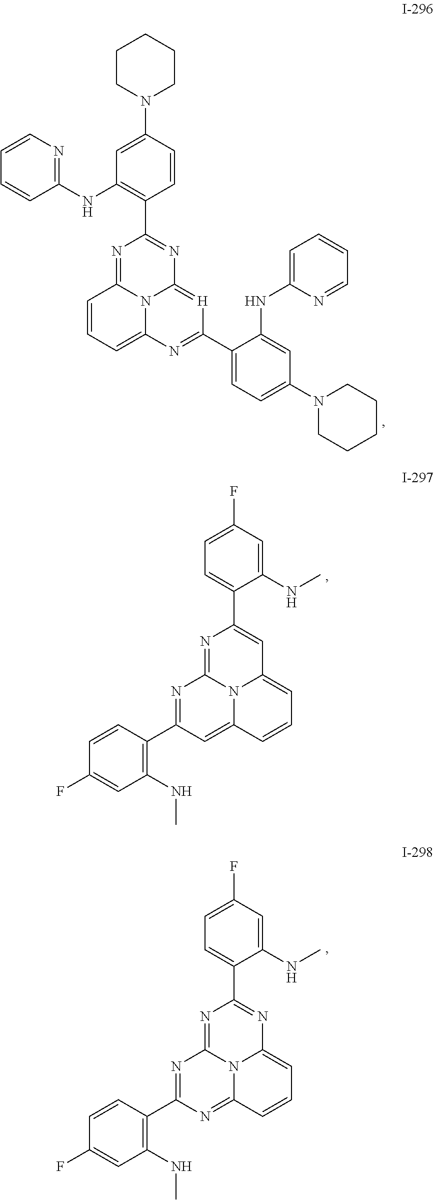

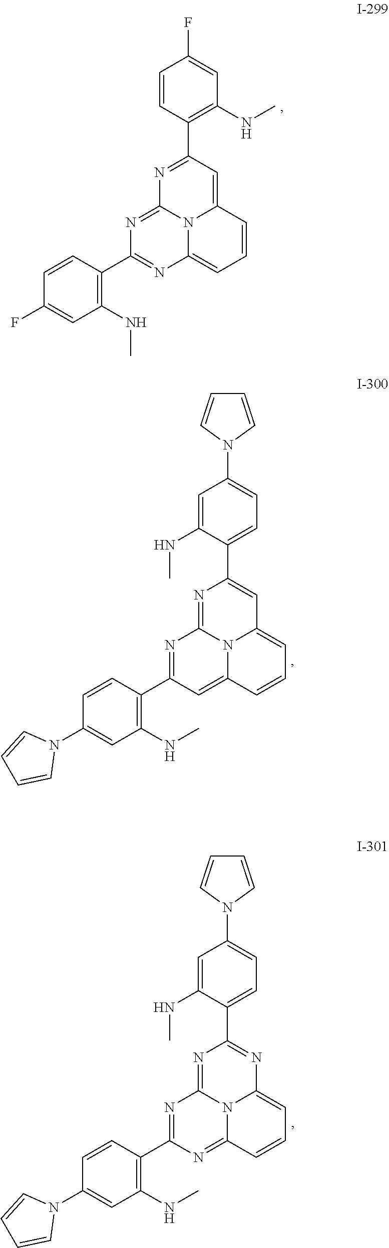 [SMILES: CNc1cc(F)ccc1C1=NC2=NC(c3ccc(F)cc3NC)=NC3=CC=CC(=C1)N32 ; CNc1cc(-n2cccc2)ccc1C1=CC2=CC=CC3=CC(c4ccc(-n5cccc5)cc4NC)=NC(=N1)N23 ; CNc1cc(-n2cccc2)ccc1C1=NC2=CC=CC3=NC(c4ccc(-n5cccc5)cc4NC)=NC(=N1)N23]

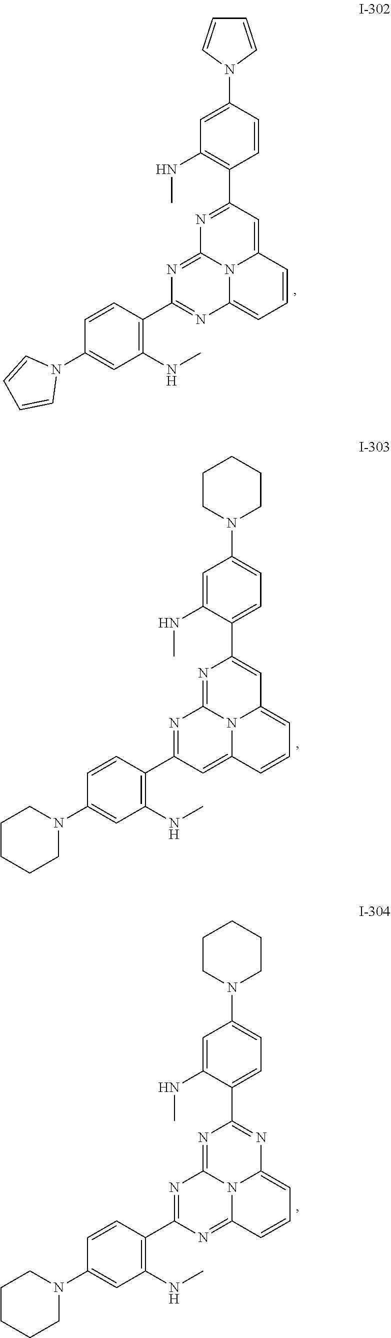 [SMILES: CNc1cc(-n2cccc2)ccc1C1=NC2=NC(c3ccc(-n4cccc4)cc3NC)=NC3=CC=CC(=C1)N32 ; CNc1cc(N2CCCCC2)ccc1C1=CC2=CC=CC3=CC(c4ccc(N5CCCCC5)cc4NC)=NC(=N1)N23 ; CNc1cc(N2CCCCC2)ccc1C1=NC2=CC=CC3=NC(c4ccc(N5CCCCC5)cc4NC)=NC(=N1)N23]

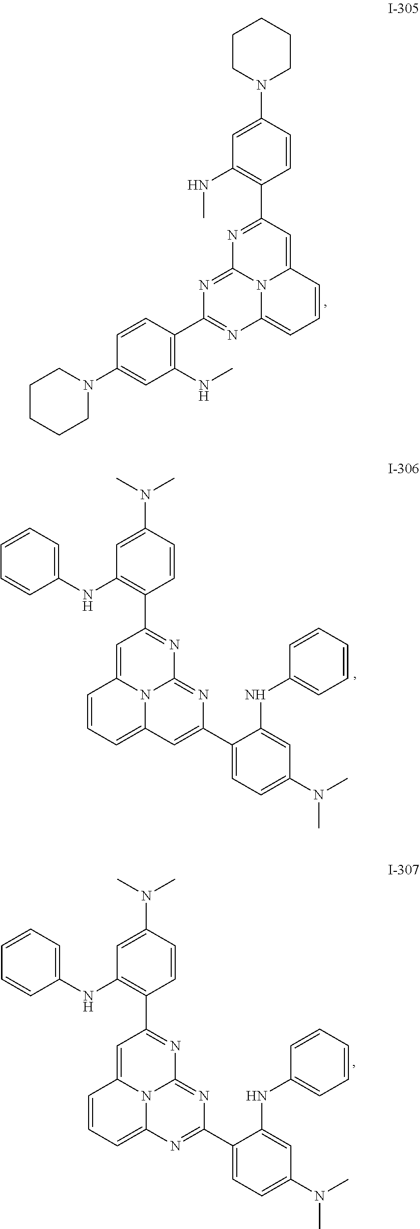 [SMILES: CNc1cc(N2CCCCC2)ccc1C1=NC2=NC(c3ccc(N4CCCCC4)cc3NC)=NC3=CC=CC(=C1)N32 ; CN(C)c1ccc(C2=CC3=CC=CC4=CC(c5ccc(N(C)C)cc5Nc5ccccc5)=NC(=N2)N34)c(Nc2ccccc2)c1 ; CN(C)c1ccc(C2=NC3=NC(c4ccc(N(C)C)cc4Nc4ccccc4)=NC4=CC=CC(=C2)N43)c(Nc2ccccc2)c1]

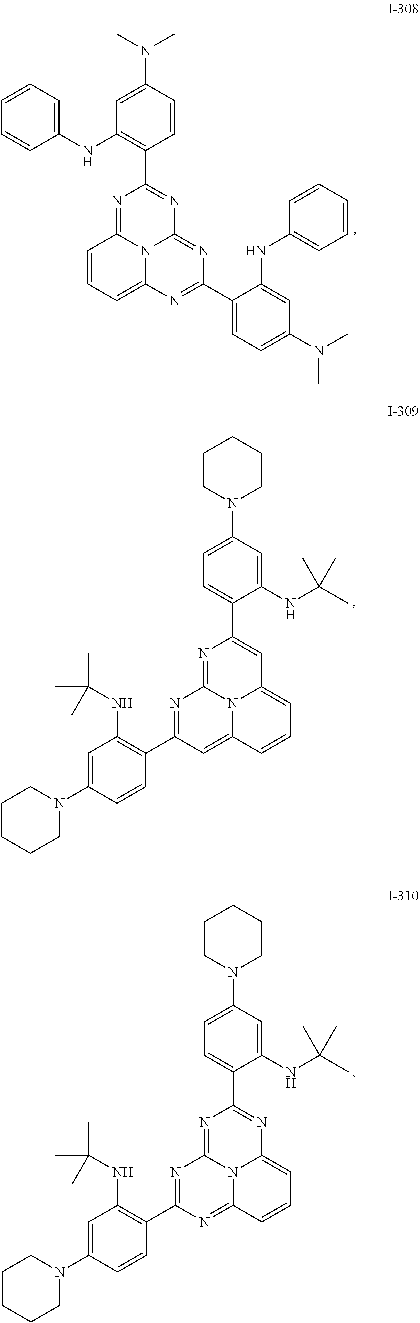 [SMILES: CN(C)c1ccc(C2=NC3=CC=CC4=NC(c5ccc(N(C)C)cc5Nc5ccccc5)=NC(=N2)N34)c(Nc2ccccc2)c1 ; CC(C)(C)Nc1cc(N2CCCCC2)ccc1C1=CC2=CC=CC3=CC(c4ccc(N5CCCCC5)cc4NC(C)(C)C)=NC(=N1)N23 ; CC(C)(C)Nc1cc(N2CCCCC2)ccc1C1=NC2=CC=CC3=NC(c4ccc(N5CCCCC5)cc4NC(C)(C)C)=NC(=N1)N23]

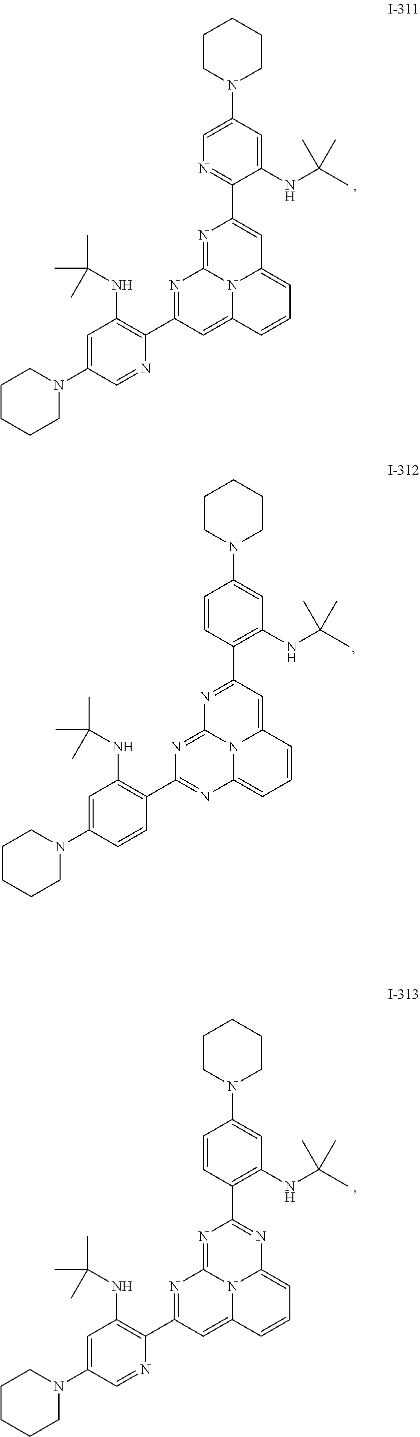 [SMILES: CC(C)(C)Nc1cc(N2CCCCC2)cnc1C1=CC2=CC=CC3=CC(c4ncc(N5CCCCC5)cc4NC(C)(C)C)=NC(=N1)N23 ; CC(C)(C)Nc1cc(N2CCCCC2)ccc1C1=NC2=NC(c3ccc(N4CCCCC4)cc3NC(C)(C)C)=NC3=CC=CC(=C1)N32 ; CC(C)(C)Nc1cc(N2CCCCC2)ccc1C1=NC2=CC=CC3=CC(c4ncc(N5CCCCC5)cc4NC(C)(C)C)=NC(=N1)N32]

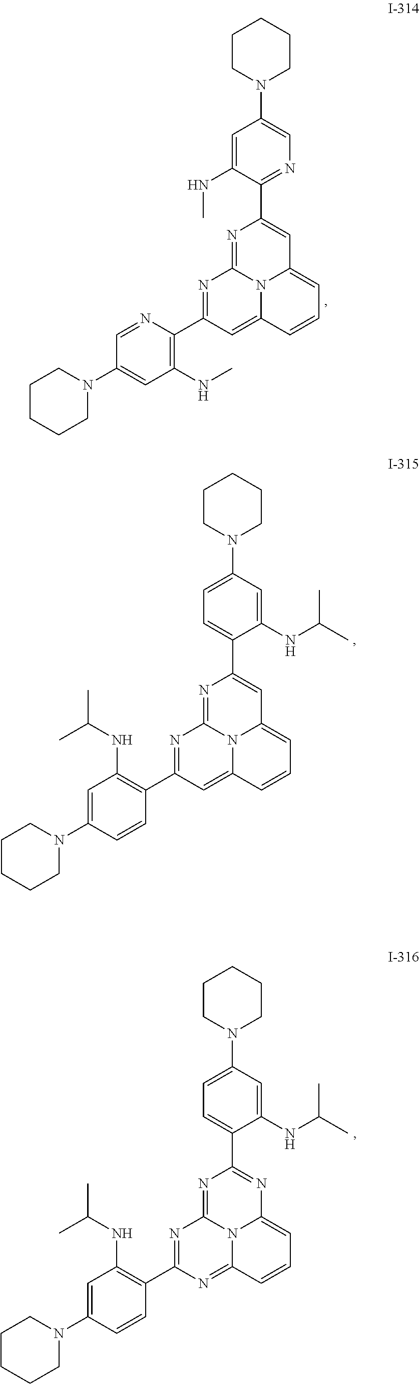 [SMILES: CNc1cc(N2CCCCC2)cnc1C1=CC2=CC=CC3=CC(c4ncc(N5CCCCC5)cc4NC)=NC(=N1)N23 ; CC(C)Nc1cc(N2CCCCC2)ccc1C1=CC2=CC=CC3=CC(c4ccc(N5CCCCC5)cc4NC(C)C)=NC(=N1)N23 ; CC(C)Nc1cc(N2CCCCC2)ccc1C1=NC2=CC=CC3=NC(c4ccc(N5CCCCC5)cc4NC(C)C)=NC(=N1)N23]

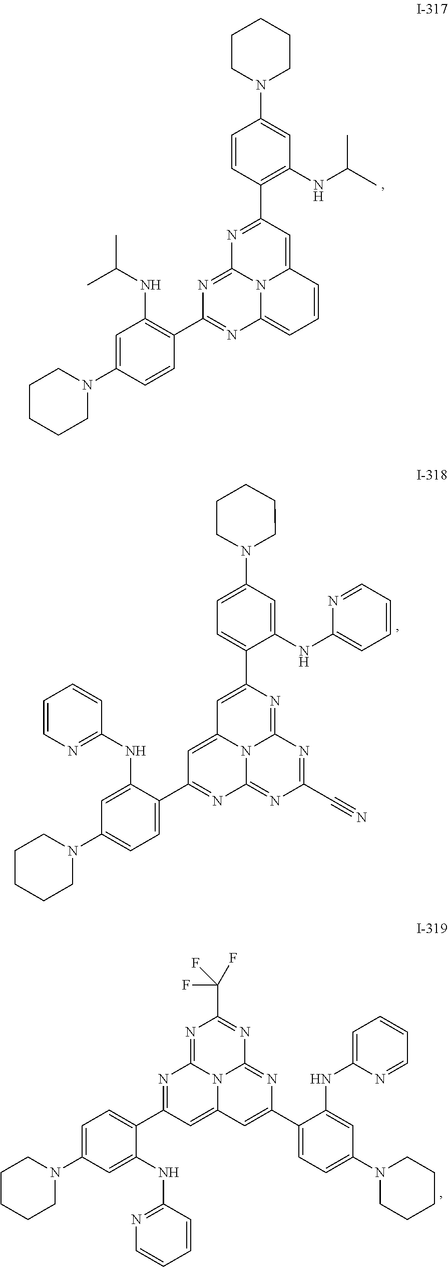 [SMILES: CC(C)Nc1cc(N2CCCCC2)ccc1C1=NC2=NC(c3ccc(N4CCCCC4)cc3NC(C)C)=NC3=CC=CC(=C1)N32 ; N#CC1=NC2=NC(c3ccc(N4CCCCC4)cc3Nc3ccccn3)=CC3=CC(c4ccc(N5CCCCC5)cc4Nc4ccccn4)=NC(=N1)N32 ; FC(F)(F)C1=NC2=NC(c3ccc(N4CCCCC4)cc3Nc3ccccn3)=CC3=CC(c4ccc(N5CCCCC5)cc4Nc4ccccn4)=NC(=N1)N32]

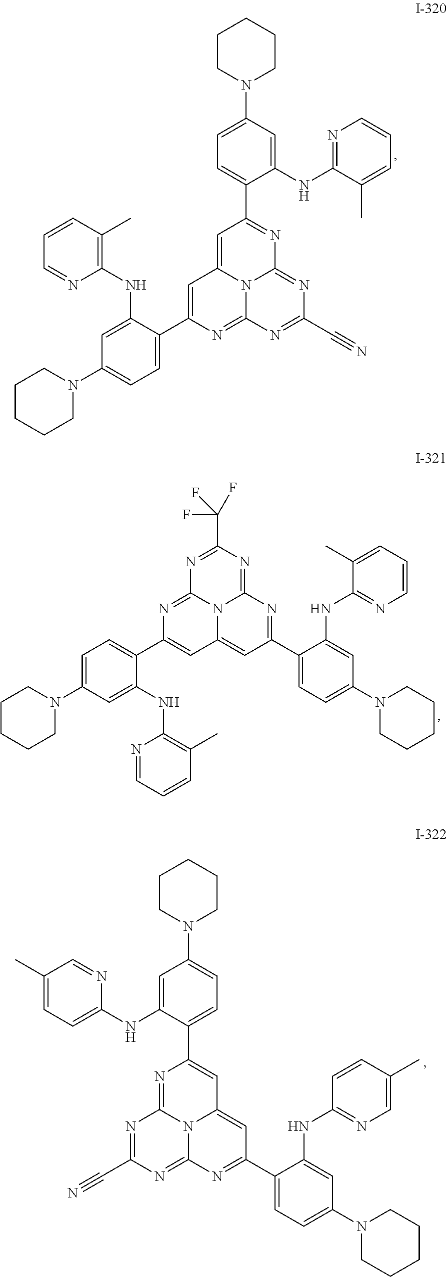 [SMILES: Cc1cccnc1Nc1cc(N2CCCCC2)ccc1C1=CC2=CC(c3ccc(N4CCCCC4)cc3Nc3ncccc3C)=NC3=NC(C#N)=NC(=N1)N23 ; Cc1cccnc1Nc1cc(N2CCCCC2)ccc1C1=CC2=CC(c3ccc(N4CCCCC4)cc3Nc3ncccc3C)=NC3=NC(C(F)(F)F)=NC(=N1)N23 ; Cc1ccc(Nc2cc(N3CCCCC3)ccc2C2=CC3=CC(c4ccc(N5CCCCC5)cc4Nc4ccc(C)cn4)=NC4=NC(C#N)=NC(=N2)N34)nc1]

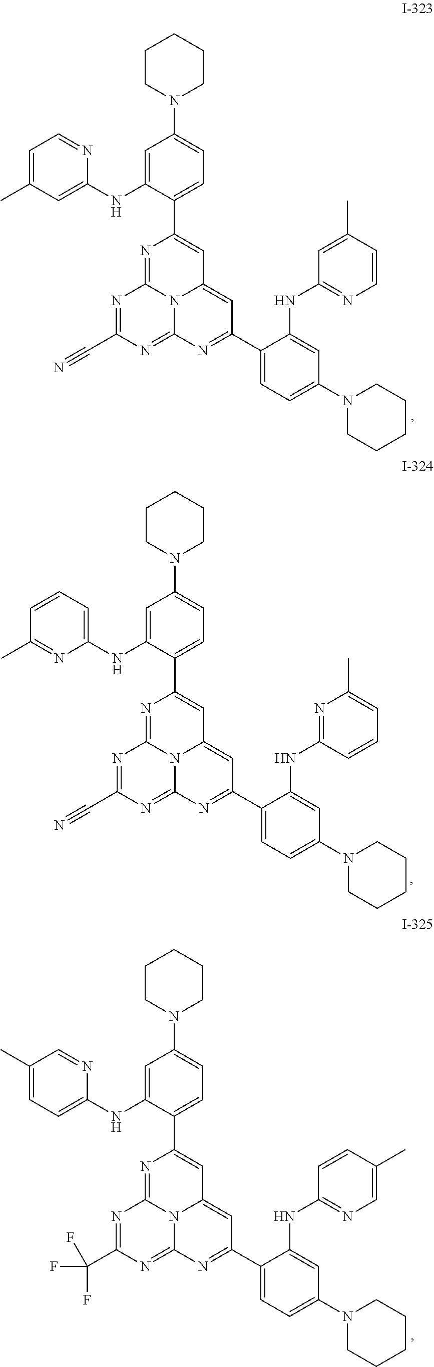

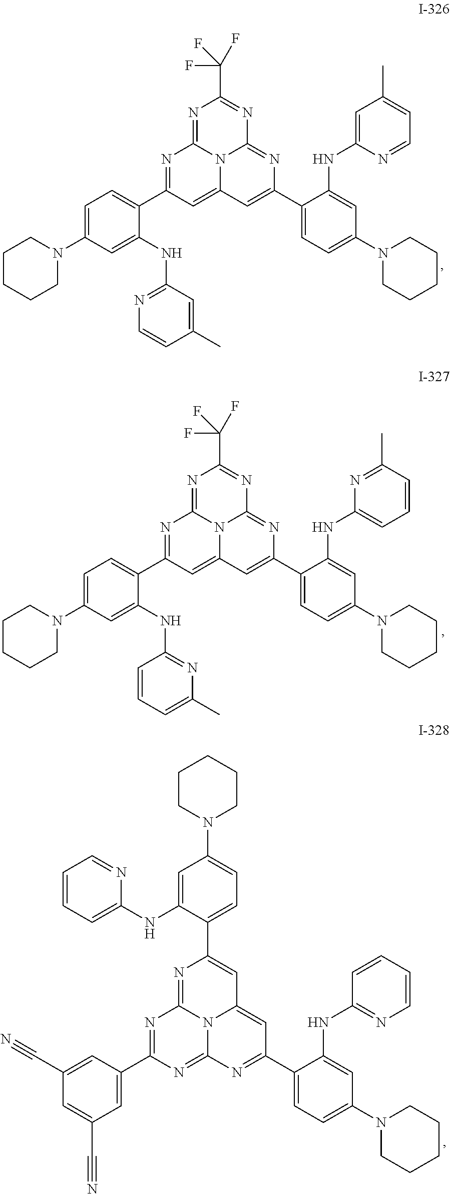

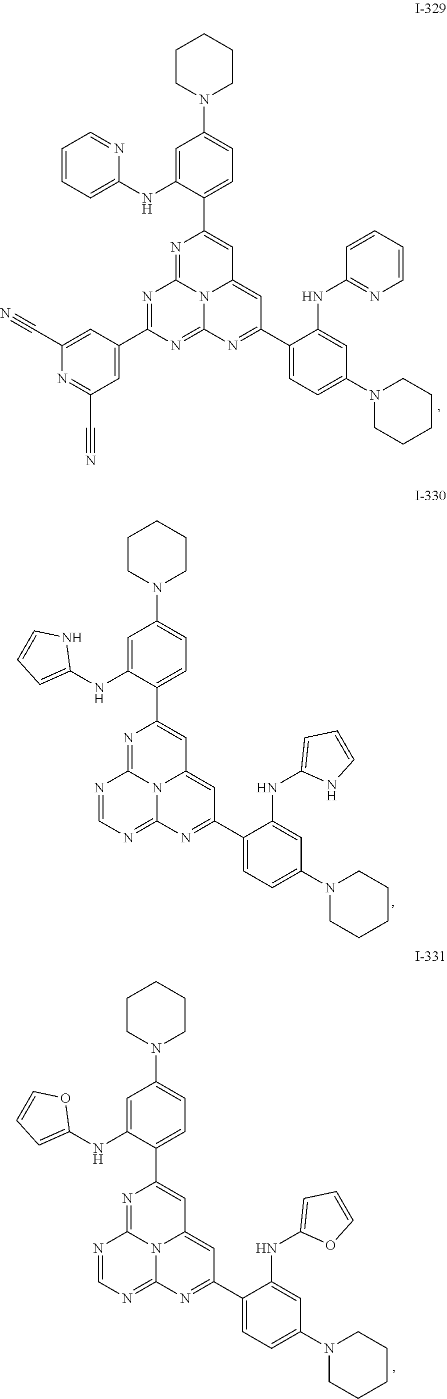 [SMILES: N#Cc1cc(C2=NC3=NC(c4ccc(N5CCCCC5)cc4Nc4ccccn4)=CC4=CC(c5ccc(N6CCCCC6)cc5Nc5ccccn5)=NC(=N2)N43)cc(C#N)n1 ; C1=NC2=NC(c3ccc(N4CCCCC4)cc3Nc3ccc[nH]3)=CC3=CC(c4ccc(N5CCCCC5)cc4Nc4ccc[nH]4)=NC(=N1)N32 ; C1=NC2=NC(c3ccc(N4CCCCC4)cc3Nc3ccco3)=CC3=CC(c4ccc(N5CCCCC5)cc4Nc4ccco4)=NC(=N1)N32]

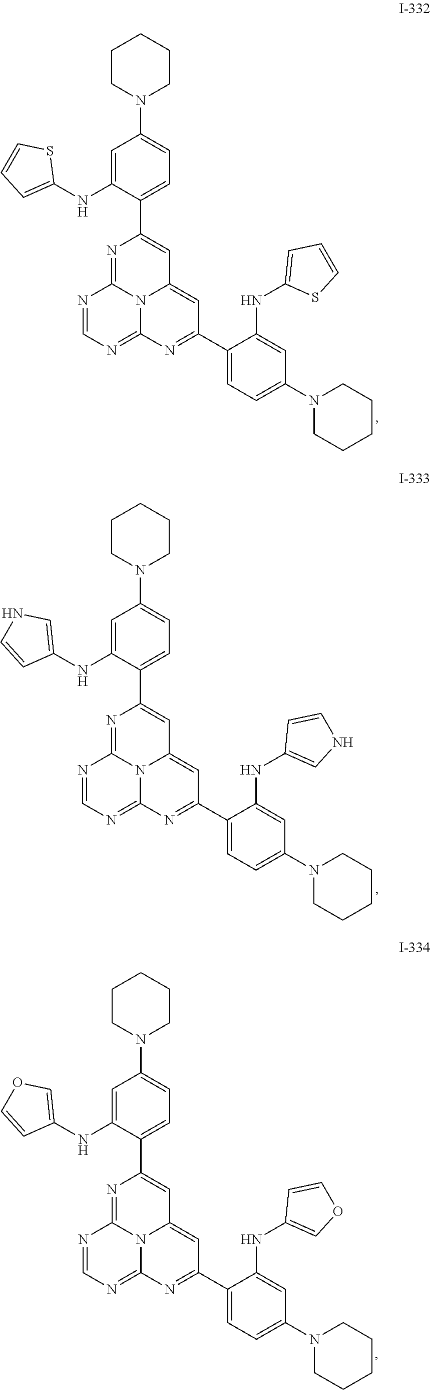 [SMILES: C1=NC2=NC(c3ccc(N4CCCCC4)cc3Nc3cccs3)=CC3=CC(c4ccc(N5CCCCC5)cc4Nc4cccs4)=NC(=N1)N32 ; C1=NC2=NC(c3ccc(N4CCCCC4)cc3Nc3cc[nH]c3)=CC3=CC(c4ccc(N5CCCCC5)cc4Nc4cc[nH]c4)=NC(=N1)N32 ; C1=NC2=NC(c3ccc(N4CCCCC4)cc3Nc3ccoc3)=CC3=CC(c4ccc(N5CCCCC5)cc4Nc4ccoc4)=NC(=N1)N32]

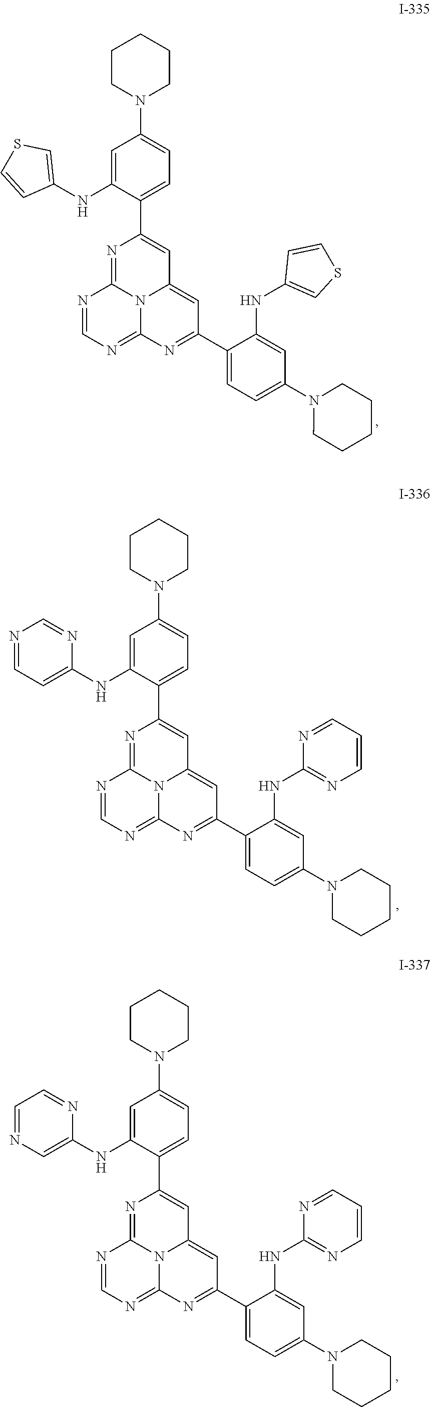 [SMILES: C1=NC2=NC(c3ccc(N4CCCCC4)cc3Nc3ccsc3)=CC3=CC(c4ccc(N5CCCCC5)cc4Nc4ccsc4)=NC(=N1)N32 ; C1=NC2=NC(c3ccc(N4CCCCC4)cc3Nc3ccncn3)=CC3=CC(c4ccc(N5CCCCC5)cc4Nc4ncccn4)=NC(=N1)N32 ; C1=NC2=NC(c3ccc(N4CCCCC4)cc3Nc3cnccn3)=CC3=CC(c4ccc(N5CCCCC5)cc4Nc4ncccn4)=NC(=N1)N32]

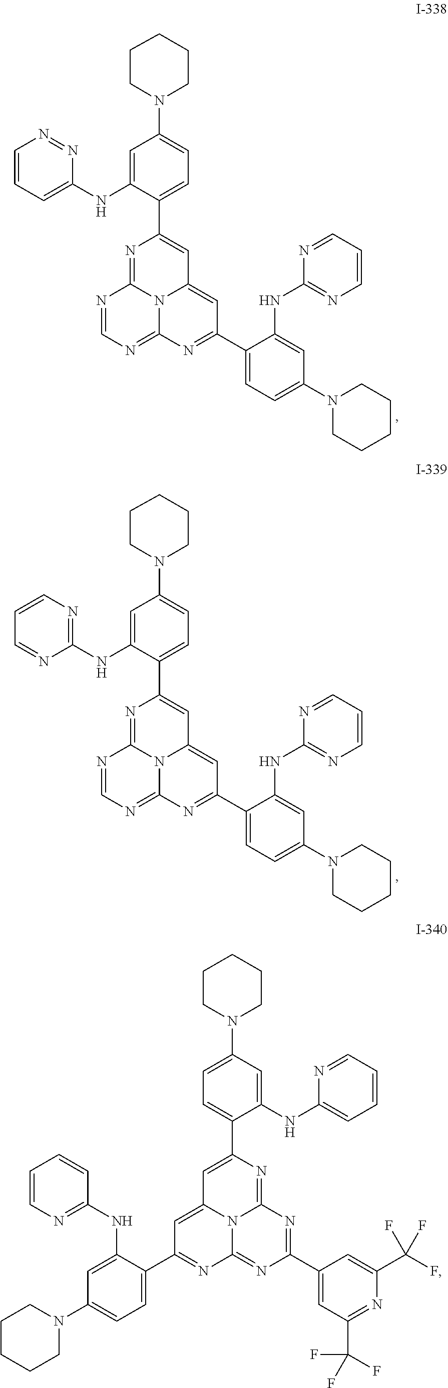 [SMILES: C1=NC2=NC(c3ccc(N4CCCCC4)cc3Nc3cccnn3)=CC3=CC(c4ccc(N5CCCCC5)cc4Nc4ncccn4)=NC(=N1)N32 ; C1=NC2=NC(c3ccc(N4CCCCC4)cc3Nc3ncccn3)=CC3=CC(c4ccc(N5CCCCC5)cc4Nc4ncccn4)=NC(=N1)N32 ; FC(F)(F)c1cc(C2=NC3=NC(c4ccc(N5CCCCC5)cc4Nc4ccccn4)=CC4=CC(c5ccc(N6CCCCC6)cc5Nc5ccccn5)=NC(=N2)N43)cc(C(F)(F)F)n1]

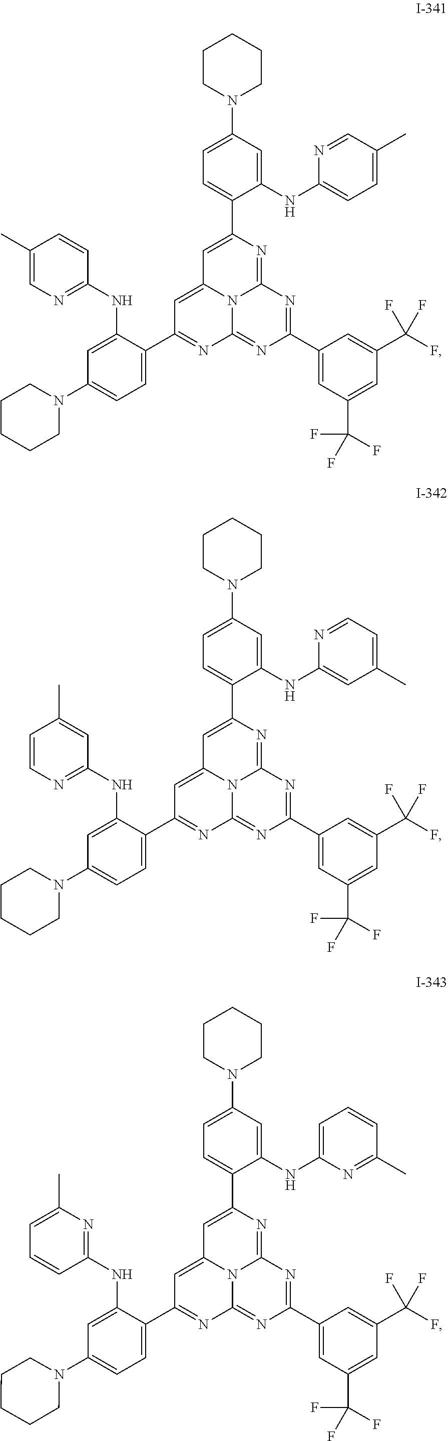 [SMILES: Cc1ccc(Nc2cc(N3CCCCC3)ccc2C2=CC3=CC(c4ccc(N5CCCCC5)cc4Nc4ccc(C)cn4)=NC4=NC(c5cc(C(F)(F)F)cc(C(F)(F)F)c5)=NC(=N2)N34)nc1 ; Cc1ccnc(Nc2cc(N3CCCCC3)ccc2C2=CC3=CC(c4ccc(N5CCCCC5)cc4Nc4cc(C)ccn4)=NC4=NC(c5cc(C(F)(F)F)cc(C(F)(F)F)c5)=NC(=N2)N34)c1 ; Cc1cccc(Nc2cc(N3CCCCC3)ccc2C2=CC3=CC(c4ccc(N5CCCCC5)cc4Nc4cccc(C)n4)=NC4=NC(c5cc(C(F)(F)F)cc(C(F)(F)F)c5)=NC(=N2)N34)n1]

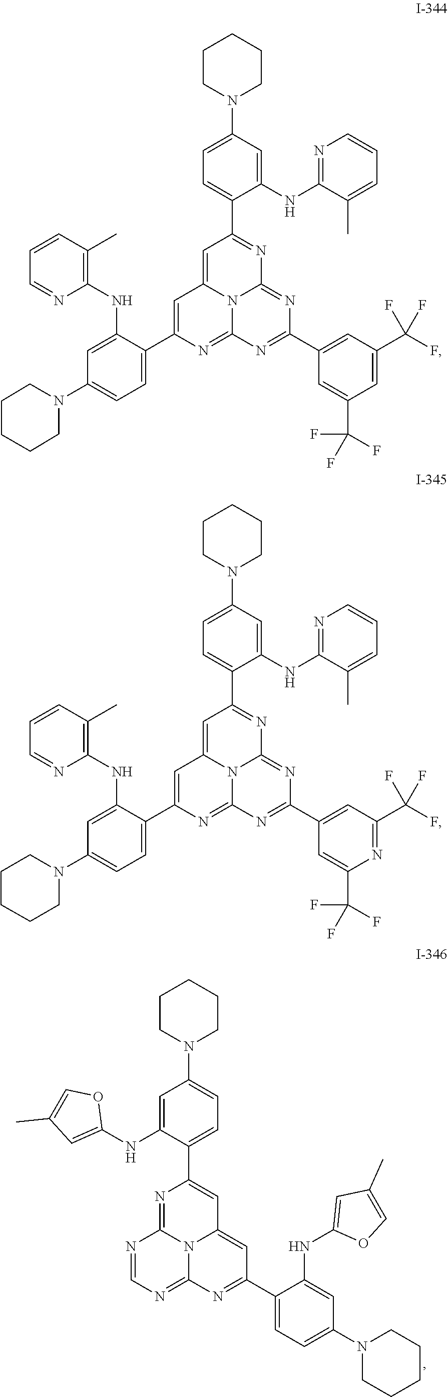 [SMILES: Cc1cccnc1Nc1cc(N2CCCCC2)ccc1C1=CC2=CC(c3ccc(N4CCCCC4)cc3Nc3ncccc3C)=NC3=NC(c4cc(C(F)(F)F)cc(C(F)(F)F)c4)=NC(=N1)N23 ; Cc1cccnc1Nc1cc(N2CCCCC2)ccc1C1=CC2=CC(c3ccc(N4CCCCC4)cc3Nc3ncccc3C)=NC3=NC(c4cc(C(F)(F)F)nc(C(F)(F)F)c4)=NC(=N1)N23 ; Cc1coc(Nc2cc(N3CCCCC3)ccc2C2=CC3=CC(c4ccc(N5CCCCC5)cc4Nc4cc(C)co4)=NC4=NC=NC(=N2)N34)c1]

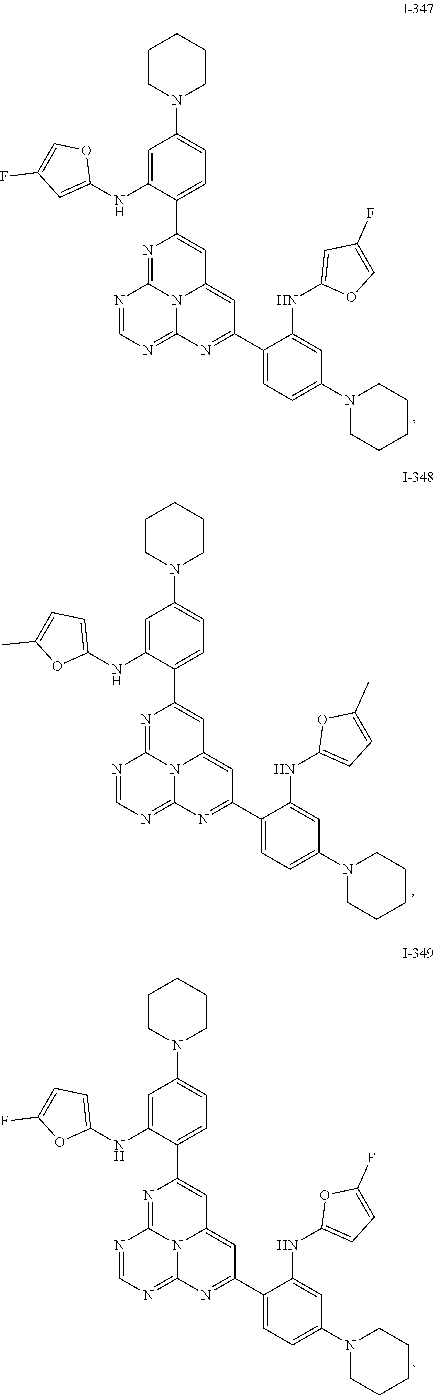 [SMILES: Fc1coc(Nc2cc(N3CCCCC3)ccc2C2=CC3=CC(c4ccc(N5CCCCC5)cc4Nc4cc(F)co4)=NC4=NC=NC(=N2)N34)c1 ; Cc1ccc(Nc2cc(N3CCCCC3)ccc2C2=CC3=CC(c4ccc(N5CCCCC5)cc4Nc4ccc(C)o4)=NC4=NC=NC(=N2)N34)o1 ; Fc1ccc(Nc2cc(N3CCCCC3)ccc2C2=CC3=CC(c4ccc(N5CCCCC5)cc4Nc4ccc(F)o4)=NC4=NC=NC(=N2)N34)o1]

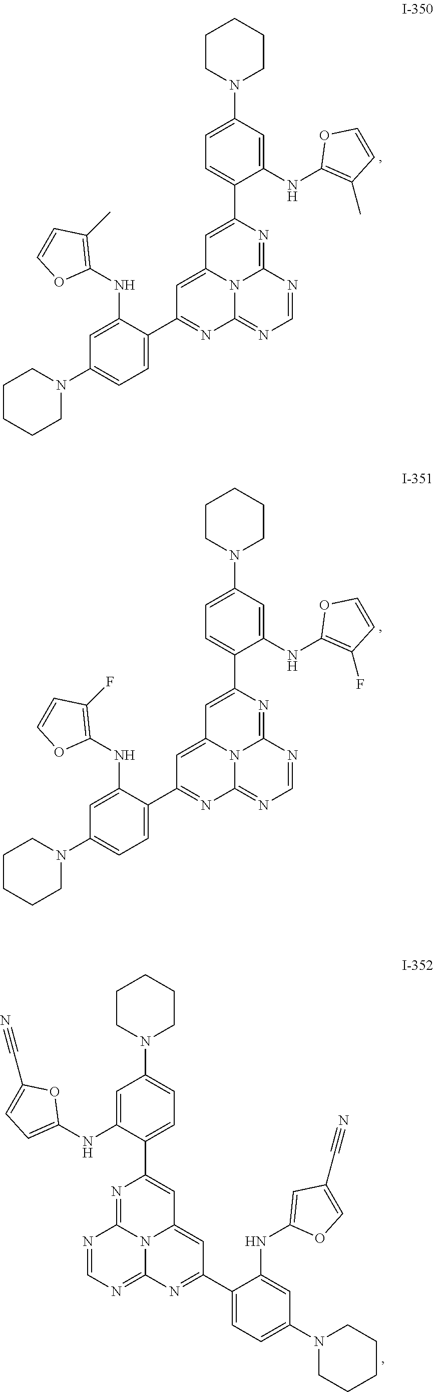 [SMILES: Cc1ccoc1Nc1cc(N2CCCCC2)ccc1C1=CC2=CC(c3ccc(N4CCCCC4)cc3Nc3occc3C)=NC3=NC=NC(=N1)N23 ; Fc1ccoc1Nc1cc(N2CCCCC2)ccc1C1=CC2=CC(c3ccc(N4CCCCC4)cc3Nc3occc3F)=NC3=NC=NC(=N1)N23 ; N#Cc1coc(Nc2cc(N3CCCCC3)ccc2C2=NC3=NC=NC4=NC(c5ccc(N6CCCCC6)cc5Nc5ccc(C#N)o5)=CC(=C2)N34)c1]

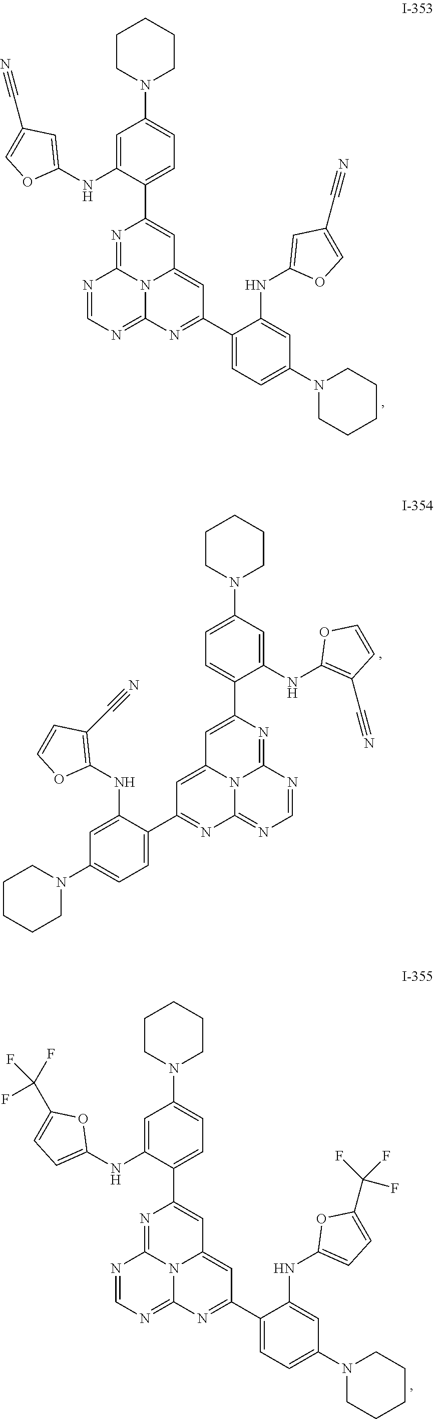 [SMILES: N#Cc1coc(Nc2cc(N3CCCCC3)ccc2C2=CC3=CC(c4ccc(N5CCCCC5)cc4Nc4cc(C#N)co4)=NC4=NC=NC(=N2)N34)c1 ; N#Cc1ccoc1Nc1cc(N2CCCCC2)ccc1C1=CC2=CC(c3ccc(N4CCCCC4)cc3Nc3occc3C#N)=NC3=NC=NC(=N1)N23 ; FC(F)(F)c1ccc(Nc2cc(N3CCCCC3)ccc2C2=CC3=CC(c4ccc(N5CCCCC5)cc4Nc4ccc(C(F)(F)F)o4)=NC4=NC=NC(=N2)N34)o1]

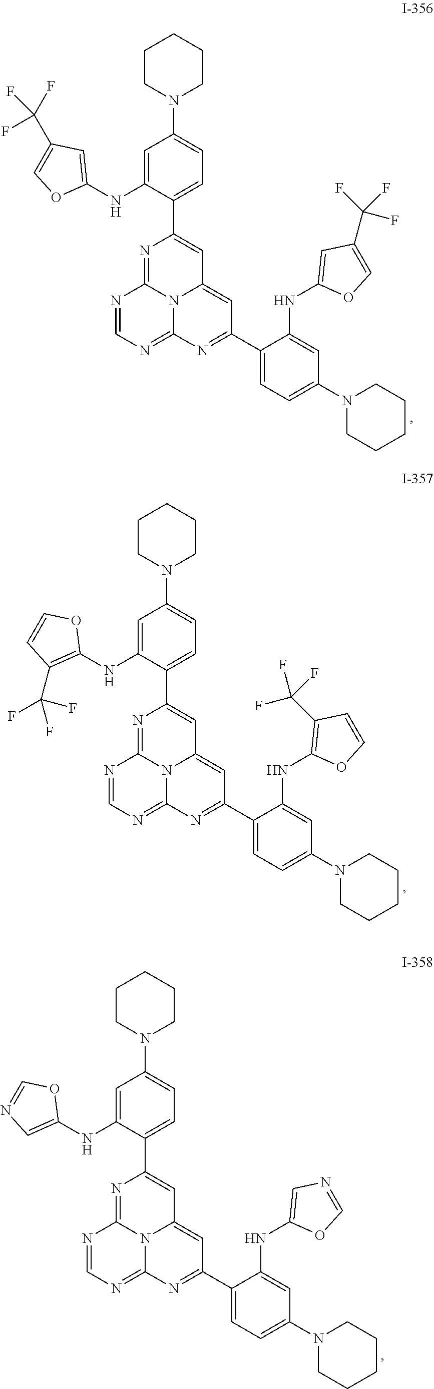 [SMILES: FC(F)(F)c1coc(Nc2cc(N3CCCCC3)ccc2C2=CC3=CC(c4ccc(N5CCCCC5)cc4Nc4cc(C(F)(F)F)co4)=NC4=NC=NC(=N2)N34)c1 ; FC(F)(F)c1ccoc1Nc1cc(N2CCCCC2)ccc1C1=CC2=CC(c3ccc(N4CCCCC4)cc3Nc3occc3C(F)(F)F)=NC3=NC=NC(=N1)N23 ; C1=NC2=NC(c3ccc(N4CCCCC4)cc3Nc3cnco3)=CC3=CC(c4ccc(N5CCCCC5)cc4Nc4cnco4)=NC(=N1)N32]

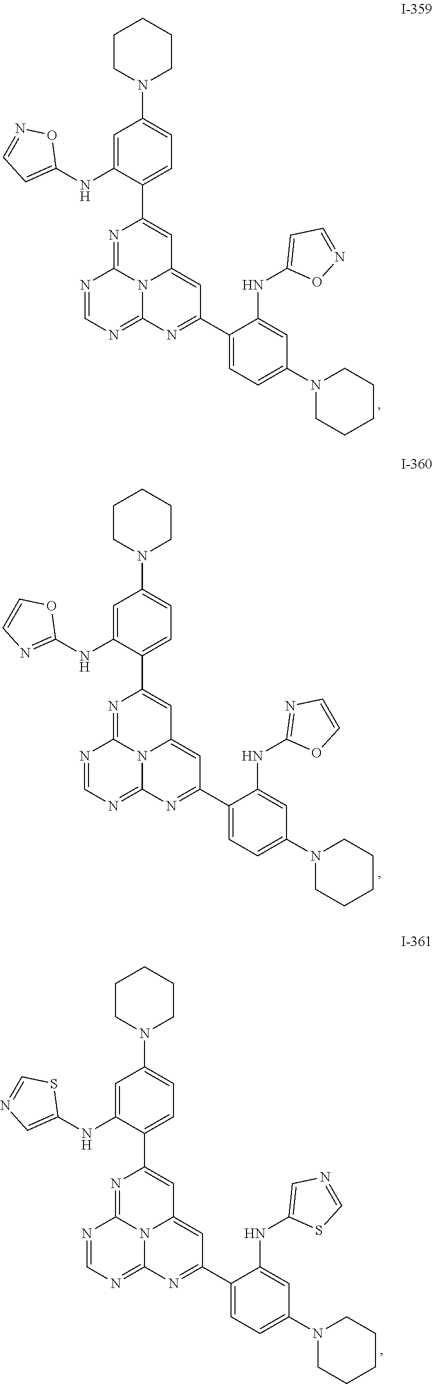

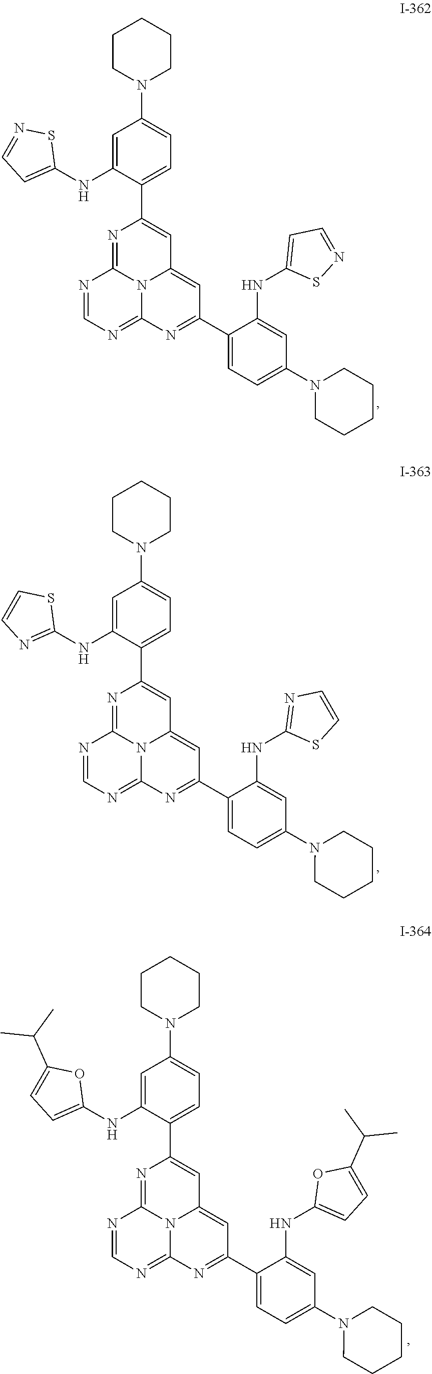 [SMILES: C1=NC2=NC(c3ccc(N4CCCCC4)cc3Nc3ccns3)=CC3=CC(c4ccc(N5CCCCC5)cc4Nc4ccns4)=NC(=N1)N32 ; C1=NC2=NC(c3ccc(N4CCCCC4)cc3Nc3nccs3)=CC3=CC(c4ccc(N5CCCCC5)cc4Nc4nccs4)=NC(=N1)N32 ; CC(C)c1ccc(Nc2cc(N3CCCCC3)ccc2C2=CC3=CC(c4ccc(N5CCCCC5)cc4Nc4ccc(C(C)C)o4)=NC4=NC=NC(=N2)N34)o1]

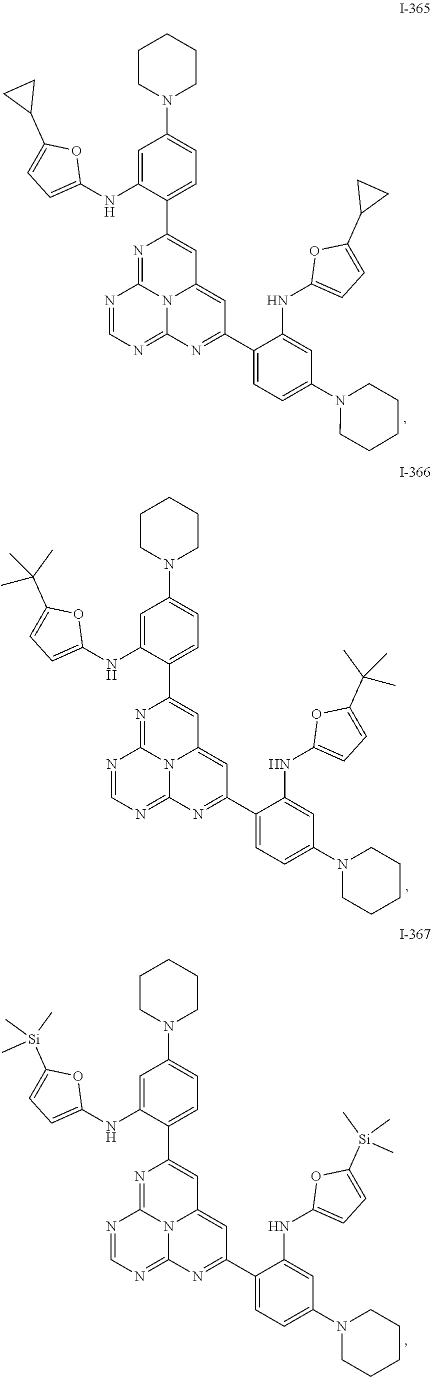 [SMILES: C1=NC2=NC(c3ccc(N4CCCCC4)cc3Nc3ccc(C4CC4)o3)=CC3=CC(c4ccc(N5CCCCC5)cc4Nc4ccc(C5CC5)o4)=NC(=N1)N32 ; CC(C)(C)c1ccc(Nc2cc(N3CCCCC3)ccc2C2=CC3=CC(c4ccc(N5CCCCC5)cc4Nc4ccc(C(C)(C)C)o4)=NC4=NC=NC(=N2)N34)o1 ; C[Si](C)(C)c1ccc(Nc2cc(N3CCCCC3)ccc2C2=CC3=CC(c4ccc(N5CCCCC5)cc4Nc4ccc([Si](C)(C)C)o4)=NC4=NC=NC(=N2)N34)o1]

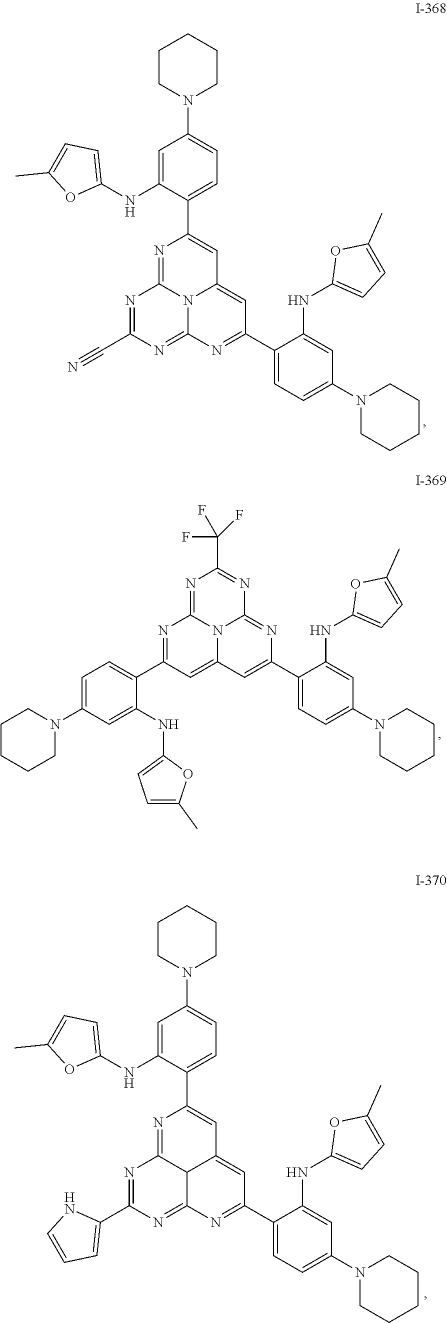 [SMILES: Cc1ccc(Nc2cc(N3CCCCC3)ccc2C2=CC3=CC(c4ccc(N5CCCCC5)cc4Nc4ccc(C)o4)=NC4=NC(C#N)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(C(F)(F)F)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ccc[nH]5)=NC(=N2)C34)o1]

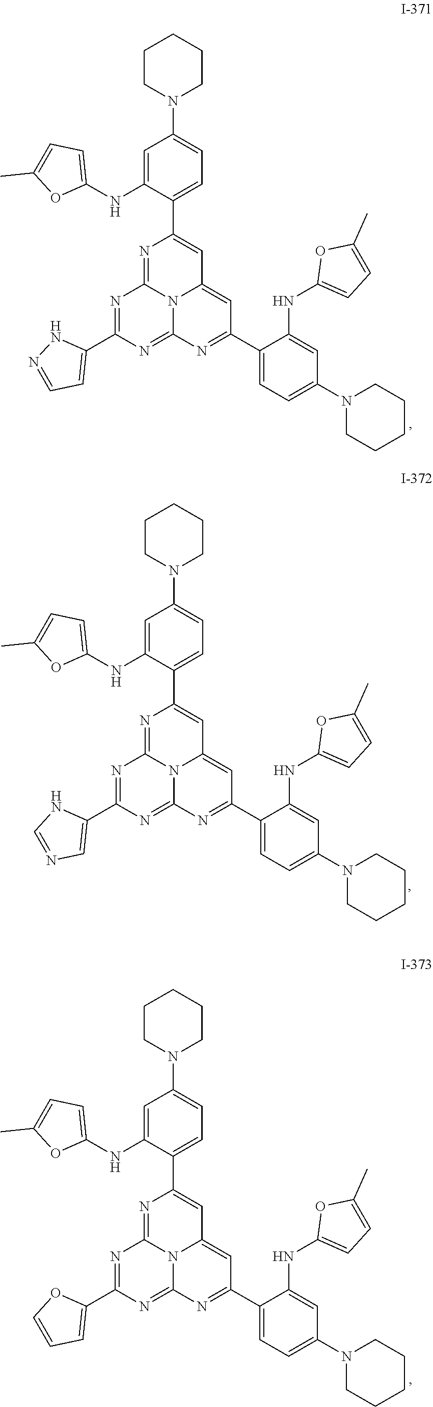

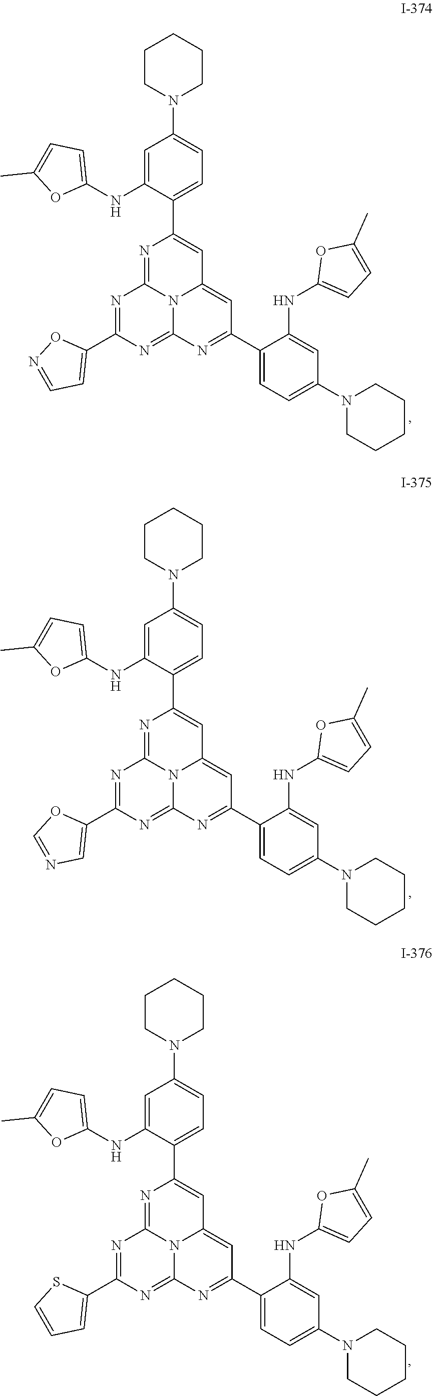

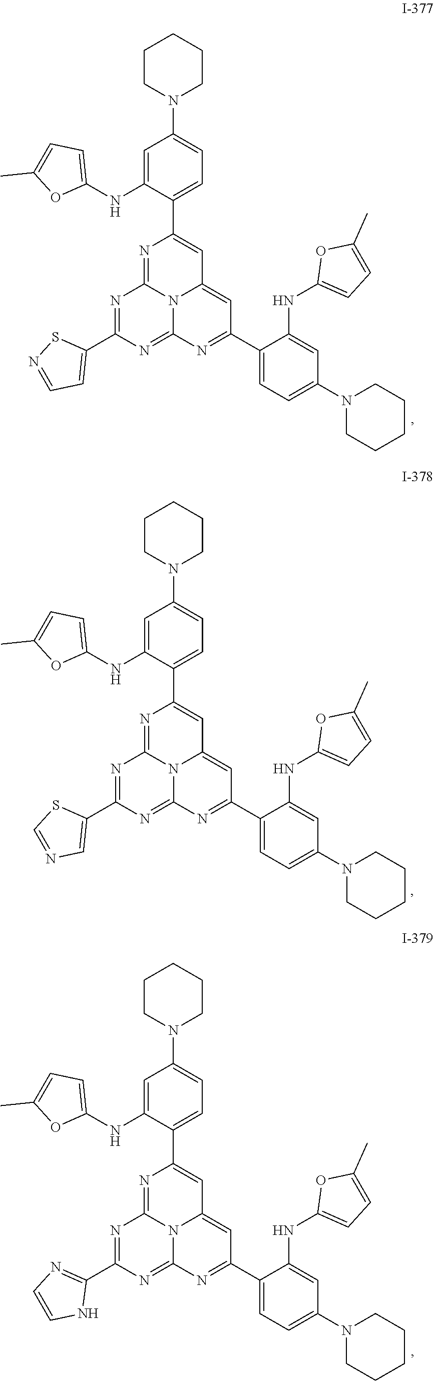 [SMILES: Cc1ccc(Nc2cc(N3CCCCC3)ccc2C2=CC3=CC(c4ccc(N5CCCCC5)cc4Nc4ccc(C)o4)=NC4=NC(c5ccns5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5cncs5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ncc[nH]5)=NC(=N2)N34)o1]

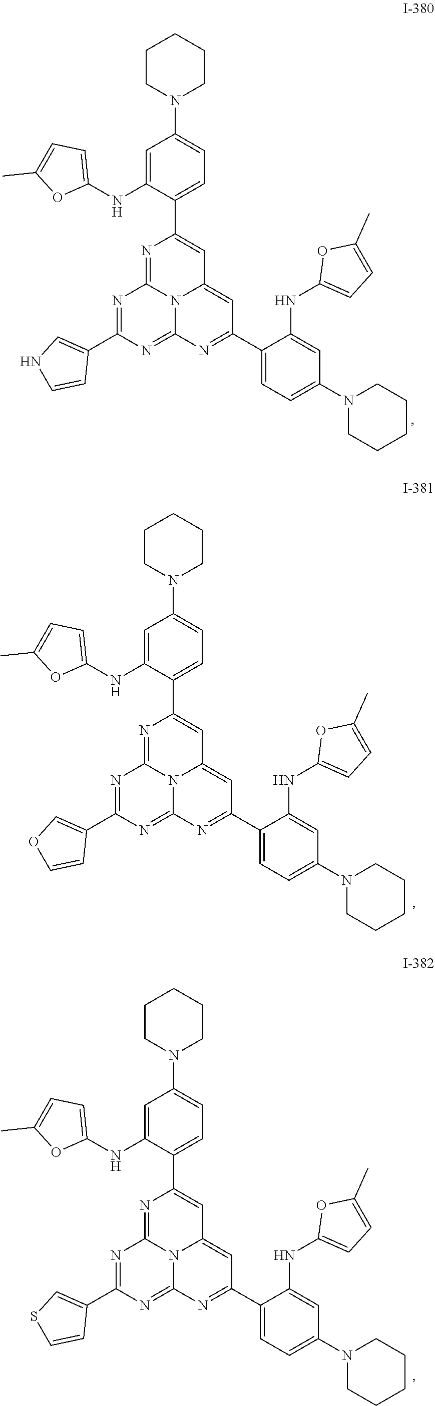 [SMILES: Cc1ccc(Nc2cc(N3CCCCC3)ccc2C2=CC3=CC(c4ccc(N5CCCCC5)cc4Nc4ccc(C)o4)=NC4=NC(c5cc[nH]c5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ccoc5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ccsc5)=NC(=N2)N34)o1]

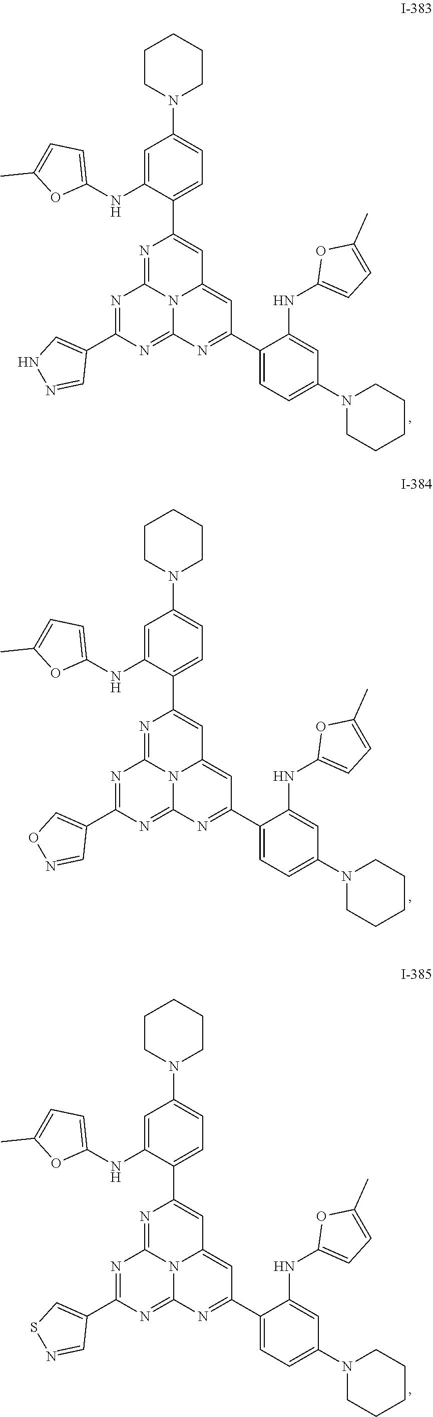 [SMILES: Cc1ccc(Nc2cc(N3CCCCC3)ccc2C2=CC3=CC(c4ccc(N5CCCCC5)cc4Nc4ccc(C)o4)=NC4=NC(c5cn[nH]c5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5cnoc5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5cnsc5)=NC(=N2)N34)o1]

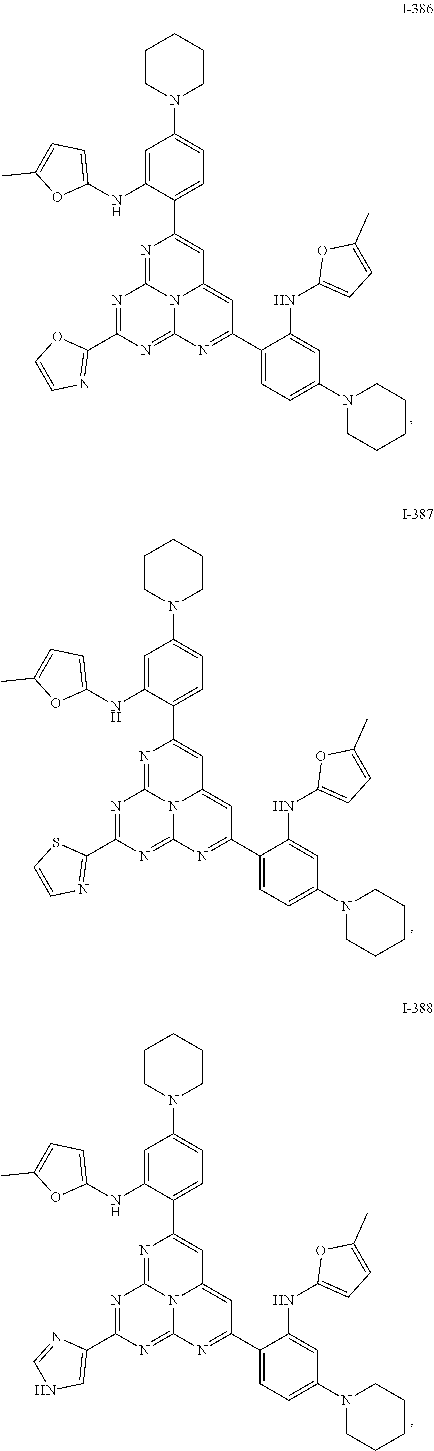 [SMILES: Cc1ccc(Nc2cc(N3CCCCC3)ccc2C2=CC3=CC(c4ccc(N5CCCCC5)cc4Nc4ccc(C)o4)=NC4=NC(c5ncco5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5nccs5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5c[nH]cn5)=NC(=N2)N34)o1]

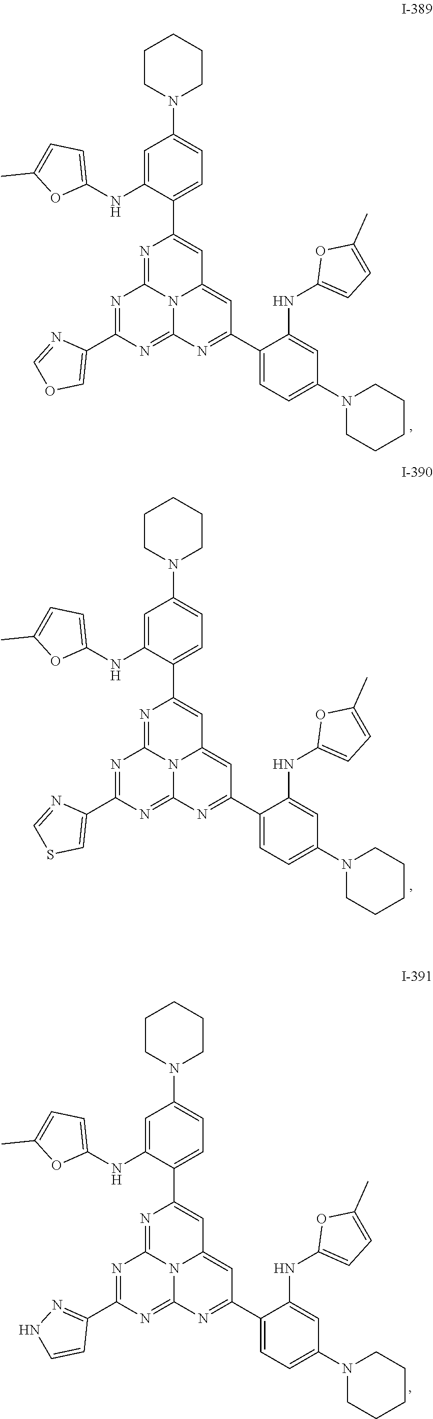

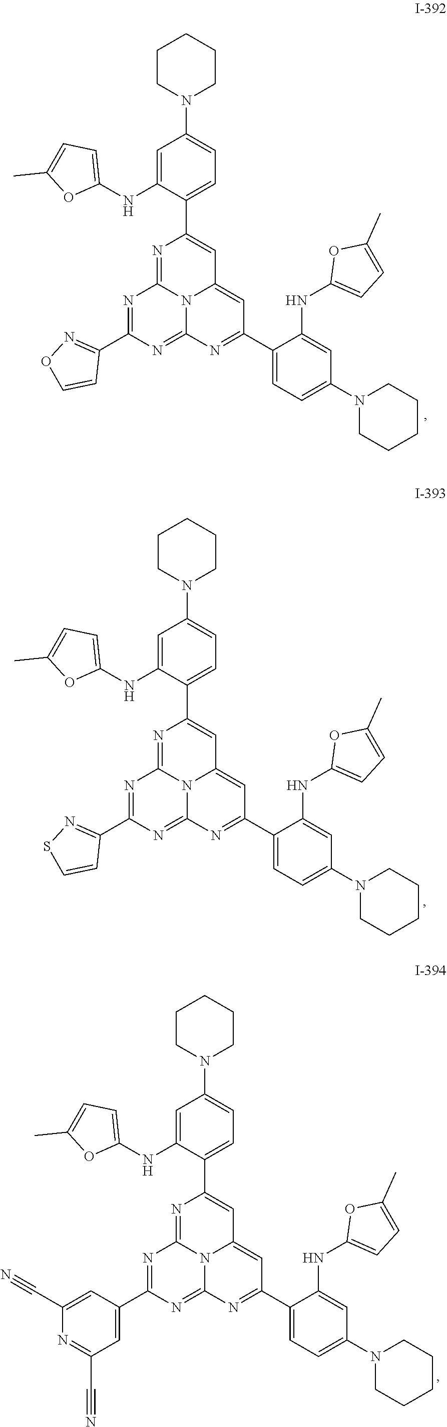 [SMILES: Cc1ccc(Nc2cc(N3CCCCC3)ccc2C2=CC3=CC(c4ccc(N5CCCCC5)cc4Nc4ccc(C)o4)=NC4=NC(c5ccon5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ccsn5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5cc(C#N)nc(C#N)c5)=NC(=N2)N34)o1]

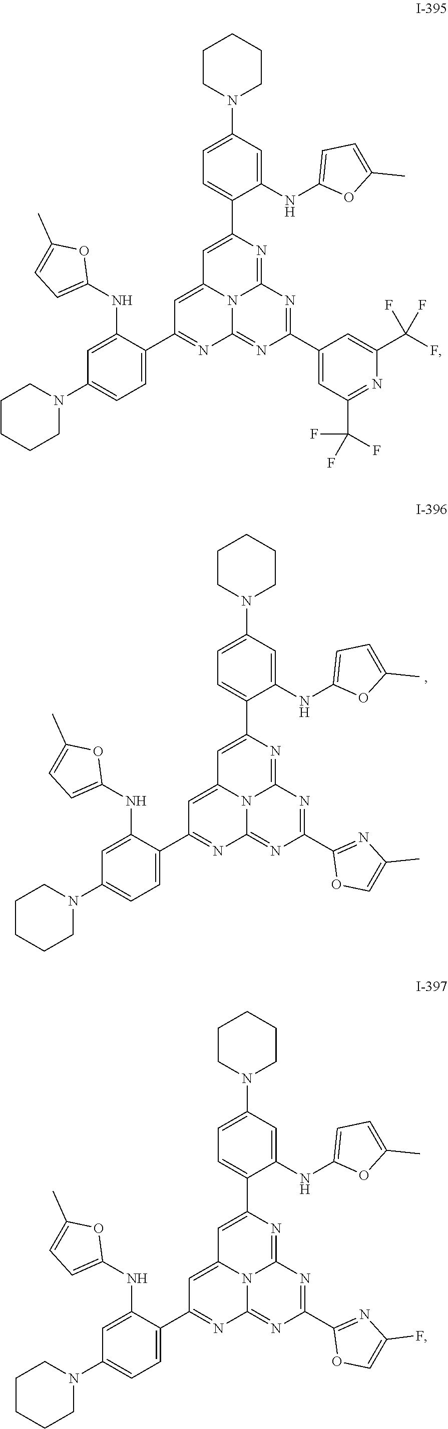 [SMILES: Cc1ccc(Nc2cc(N3CCCCC3)ccc2C2=CC3=CC(c4ccc(N5CCCCC5)cc4Nc4ccc(C)o4)=NC4=NC(c5cc(C(F)(F)F)nc(C(F)(F)F)c5)=NC(=N2)N34)o1 ; Cc1coc(C2=NC3=NC(c4ccc(N5CCCCC5)cc4Nc4ccc(C)o4)=CC4=CC(c5ccc(N6CCCCC6)cc5Nc5ccc(C)o5)=NC(=N2)N43)n1 ; Cc1ccc(Nc2cc(N3CCCCC3)ccc2C2=CC3=CC(c4ccc(N5CCCCC5)cc4Nc4ccc(C)o4)=NC4=NC(c5nc(F)co5)=NC(=N2)N34)o1]

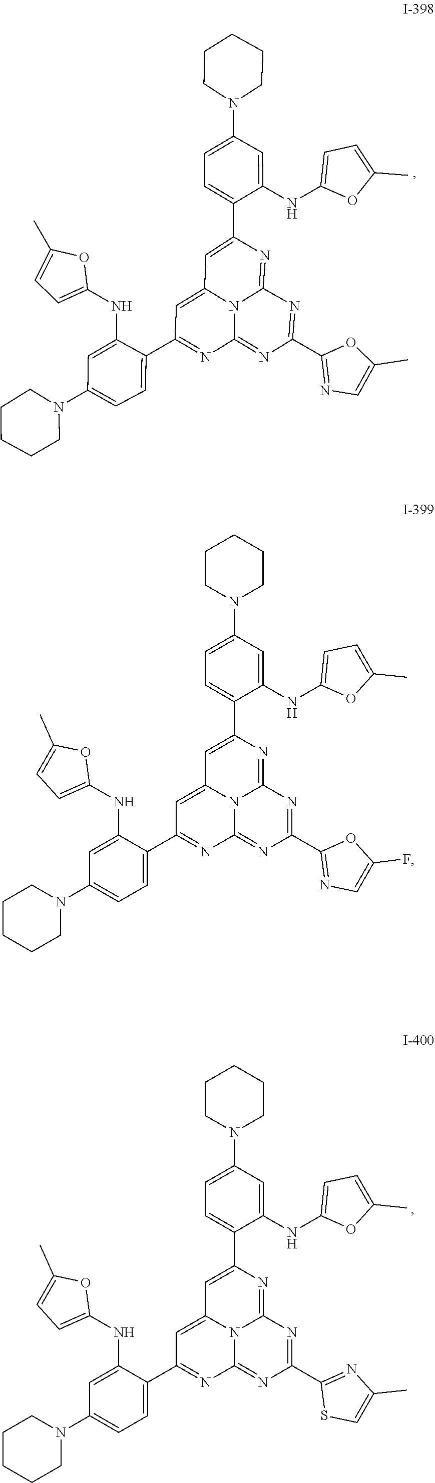 [SMILES: Cc1ccc(Nc2cc(N3CCCCC3)ccc2C2=CC3=CC(c4ccc(N5CCCCC5)cc4Nc4ccc(C)o4)=NC4=NC(c5ncc(C)o5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ncc(F)o5)=NC(=N2)N34)o1 ; Cc1csc(C2=NC3=NC(c4ccc(N5CCCCC5)cc4Nc4ccc(C)o4)=CC4=CC(c5ccc(N6CCCCC6)cc5Nc5ccc(C)o5)=NC(=N2)N43)n1]

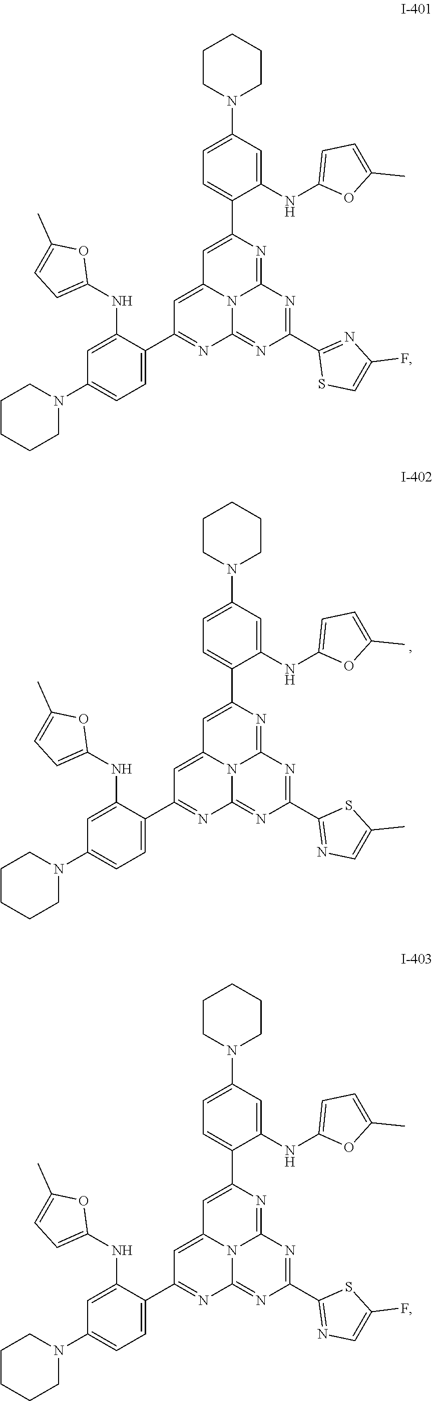 [SMILES: Cc1ccc(Nc2cc(N3CCCCC3)ccc2C2=CC3=CC(c4ccc(N5CCCCC5)cc4Nc4ccc(C)o4)=NC4=NC(c5nc(F)cs5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ncc(C)s5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ncc(F)s5)=NC(=N2)N34)o1]

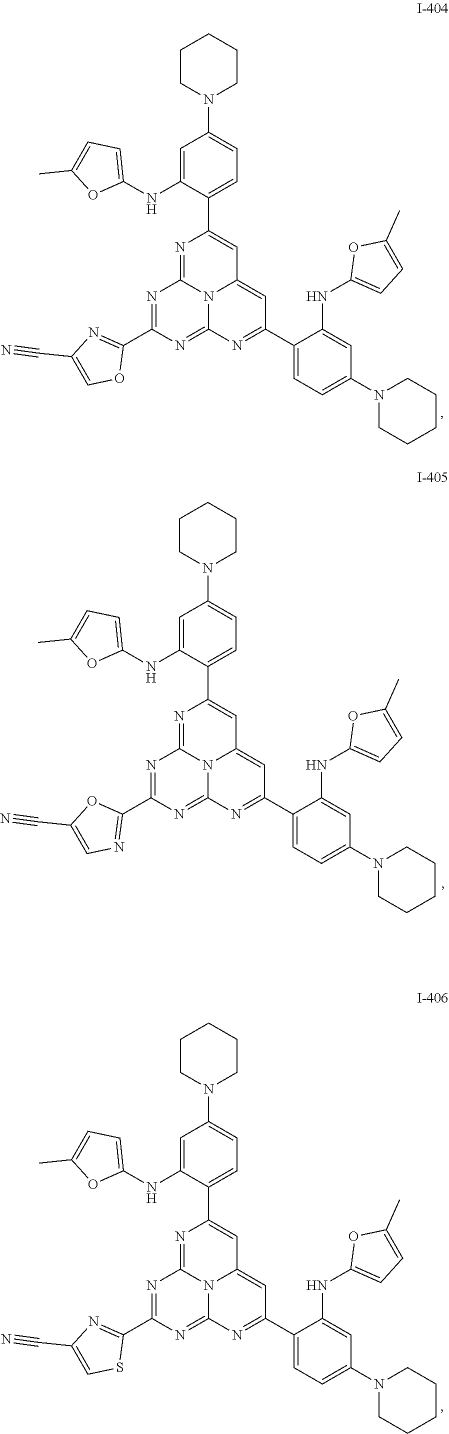 [SMILES: Cc1ccc(Nc2cc(N3CCCCC3)ccc2C2=CC3=CC(c4ccc(N5CCCCC5)cc4Nc4ccc(C)o4)=NC4=NC(c5nc(C#N)co5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ncc(C#N)o5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5nc(C#N)cs5)=NC(=N2)N34)o1]

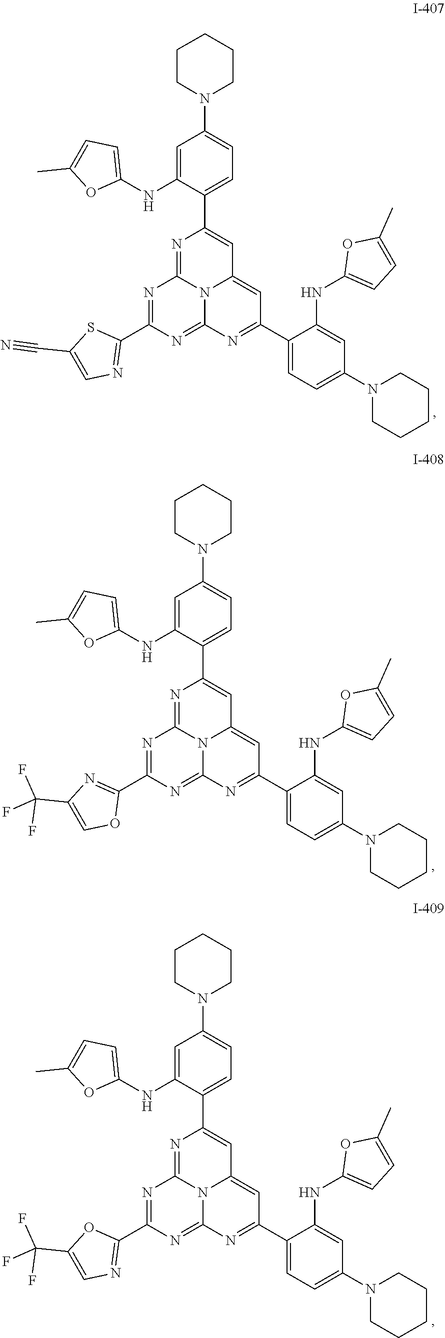 [SMILES: Cc1ccc(Nc2cc(N3CCCCC3)ccc2C2=CC3=CC(c4ccc(N5CCCCC5)cc4Nc4ccc(C)o4)=NC4=NC(c5ncc(C#N)s5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5nc(C(F)(F)F)co5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ncc(C(F)(F)F)o5)=NC(=N2)N34)o1]

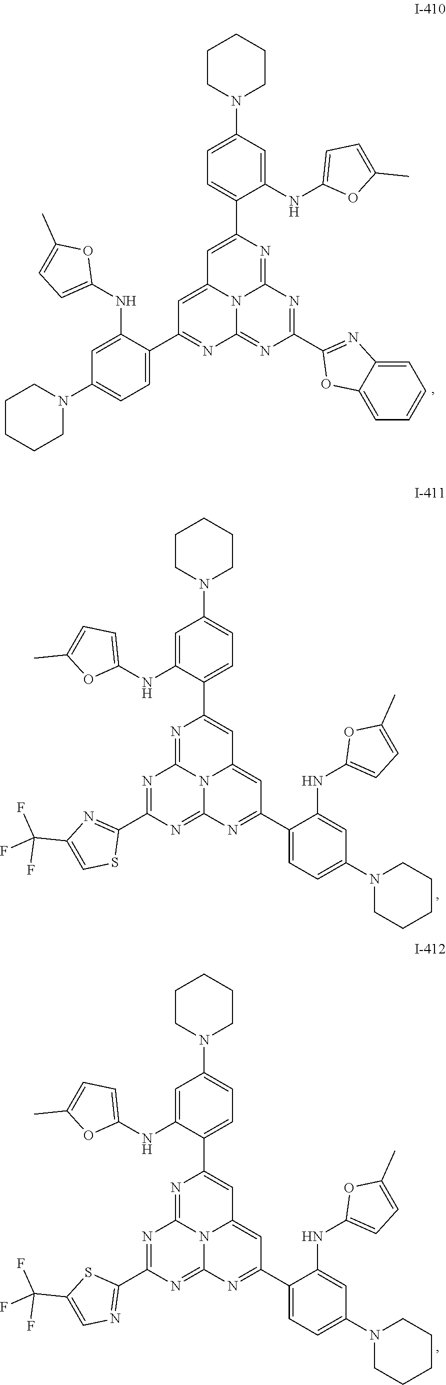 [SMILES: Cc1ccc(Nc2cc(N3CCCCC3)ccc2C2=CC3=CC(c4ccc(N5CCCCC5)cc4Nc4ccc(C)o4)=NC4=NC(c5nc6ccccc6o5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5nc(C(F)(F)F)cs5)=NC(=N2)N34)o1 ; Cc1ccc(Nc2cc(N3CCCCC3)ccc2C2=CC3=CC(c4ccc(N5CCCCC5)cc4Nc4ccc(C)o4)=NC4=NC(c5ncc(C(F)(F)F)s5)=NC(=N2)N34)o1]

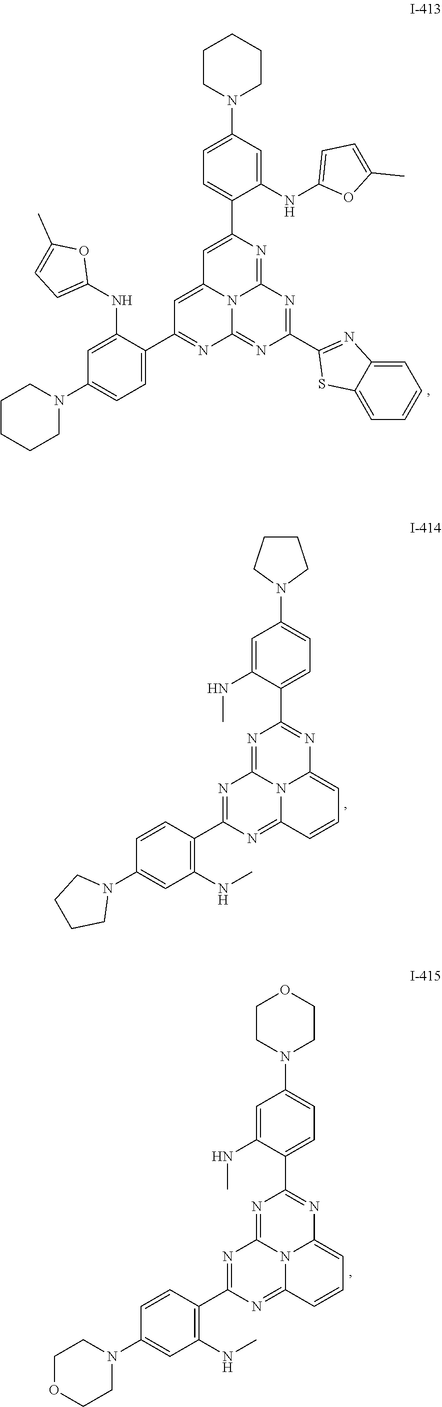 [SMILES: Cc1ccc(Nc2cc(N3CCCCC3)ccc2C2=CC3=CC(c4ccc(N5CCCCC5)cc4Nc4ccc(C)o4)=NC4=NC(c5nc6ccccc6s5)=NC(=N2)N34)o1 ; CNc1cc(N2CCCC2)ccc1C1=NC2=CC=CC3=NC(c4ccc(N5CCCC5)cc4NC)=NC(=N1)N23 ; CNc1cc(N2CCOCC2)ccc1C1=NC2=CC=CC3=NC(c4ccc(N5CCOCC5)cc4NC)=NC(=N1)N23]

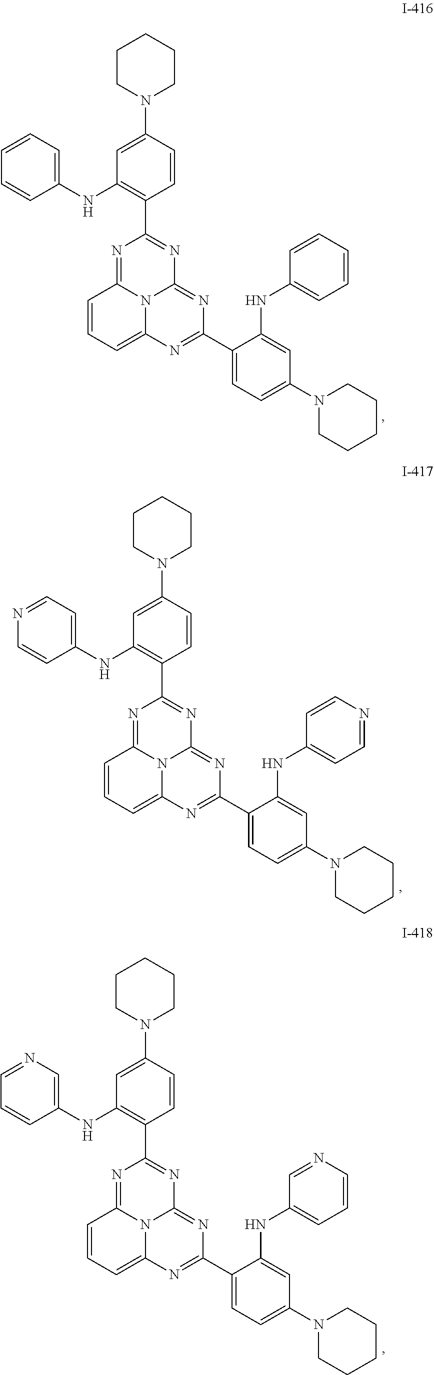 [SMILES: C1=CC2=NC(c3ccc(N4CCCCC4)cc3Nc3ccccc3)=NC3=NC(c4ccc(N5CCCCC5)cc4Nc4ccccc4)=NC(=C1)N23 ; C1=CC2=NC(c3ccc(N4CCCCC4)cc3Nc3ccncc3)=NC3=NC(c4ccc(N5CCCCC5)cc4Nc4ccncc4)=NC(=C1)N23 ; C1=CC2=NC(c3ccc(N4CCCCC4)cc3Nc3cccnc3)=NC3=NC(c4ccc(N5CCCCC5)cc4Nc4cccnc4)=NC(=C1)N23]

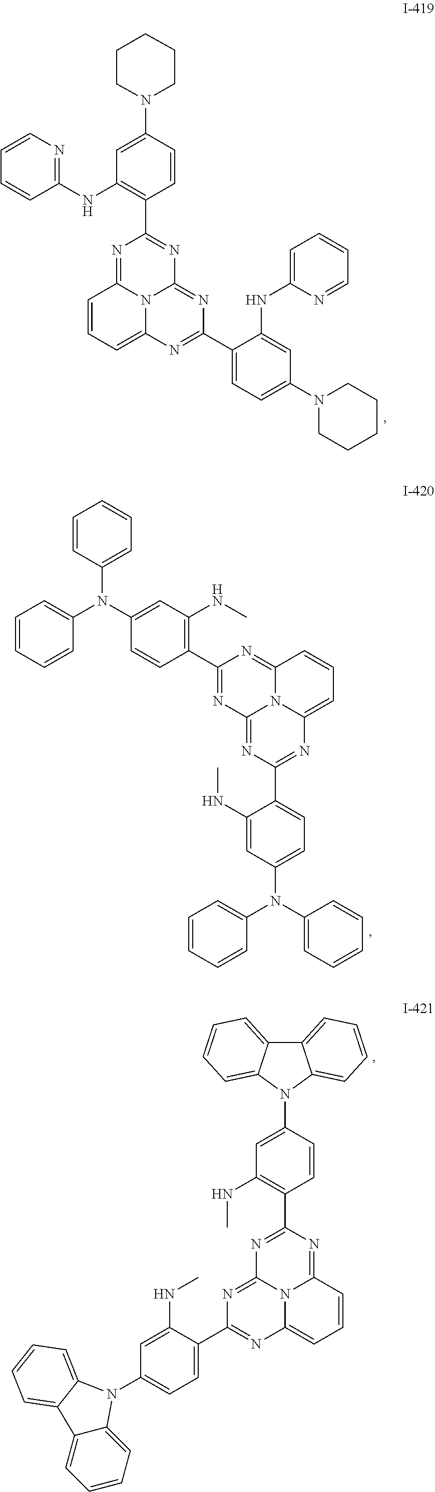 [SMILES: C1=CC2=NC(c3ccc(N4CCCCC4)cc3Nc3ccccn3)=NC3=NC(c4ccc(N5CCCCC5)cc4Nc4ccccn4)=NC(=C1)N23 ; CNc1cc(N(c2ccccc2)c2ccccc2)ccc1C1=NC2=CC=CC3=NC(c4ccc(N(c5ccccc5)c5ccccc5)cc4NC)=NC(=N1)N23 ; CNc1cc(-n2c3ccccc3c3ccccc32)ccc1C1=NC2=CC=CC3=NC(c4ccc(-n5c6ccccc6c6ccccc65)cc4NC)=NC(=N1)N23]

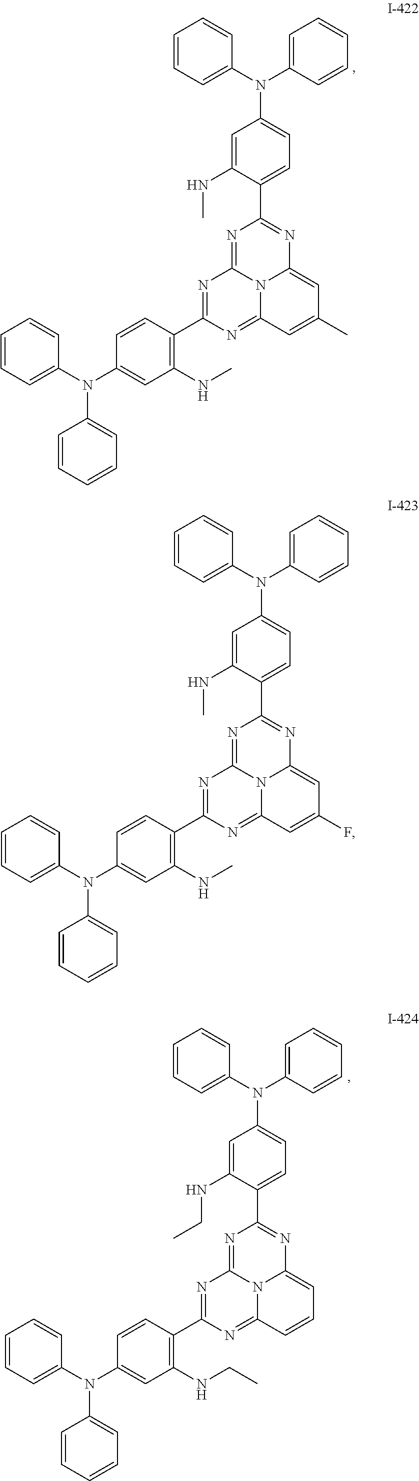 [SMILES: CNc1cc(N(c2ccccc2)c2ccccc2)ccc1C1=NC2=CC(C)=CC3=NC(c4ccc(N(c5ccccc5)c5ccccc5)cc4NC)=NC(=N1)N23 ; CNc1cc(N(c2ccccc2)c2ccccc2)ccc1C1=NC2=CC(F)=CC3=NC(c4ccc(N(c5ccccc5)c5ccccc5)cc4NC)=NC(=N1)N23 ; CCNc1cc(N(c2ccccc2)c2ccccc2)ccc1C1=NC2=CC=CC3=NC(c4ccc(N(c5ccccc5)c5ccccc5)cc4NCC)=NC(=N1)N23]

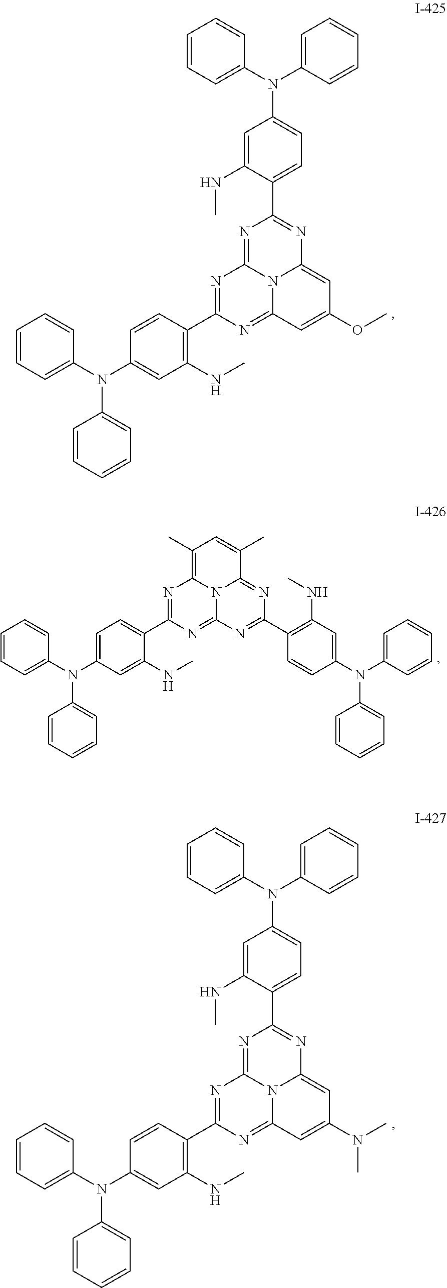 [SMILES: CNc1cc(N(c2ccccc2)c2ccccc2)ccc1C1=NC2=CC(OC)=CC3=NC(c4ccc(N(c5ccccc5)c5ccccc5)cc4NC)=NC(=N1)N23 ; CNc1cc(N(c2ccccc2)c2ccccc2)ccc1C1=NC2=NC(c3ccc(N(c4ccccc4)c4ccccc4)cc3NC)=NC3=C(C)C=C(C)C(=N1)N23 ; CNc1cc(N(c2ccccc2)c2ccccc2)ccc1C1=NC2=CC(N(C)C)=CC3=NC(c4ccc(N(c5ccccc5)c5ccccc5)cc4NC)=NC(=N1)N23]

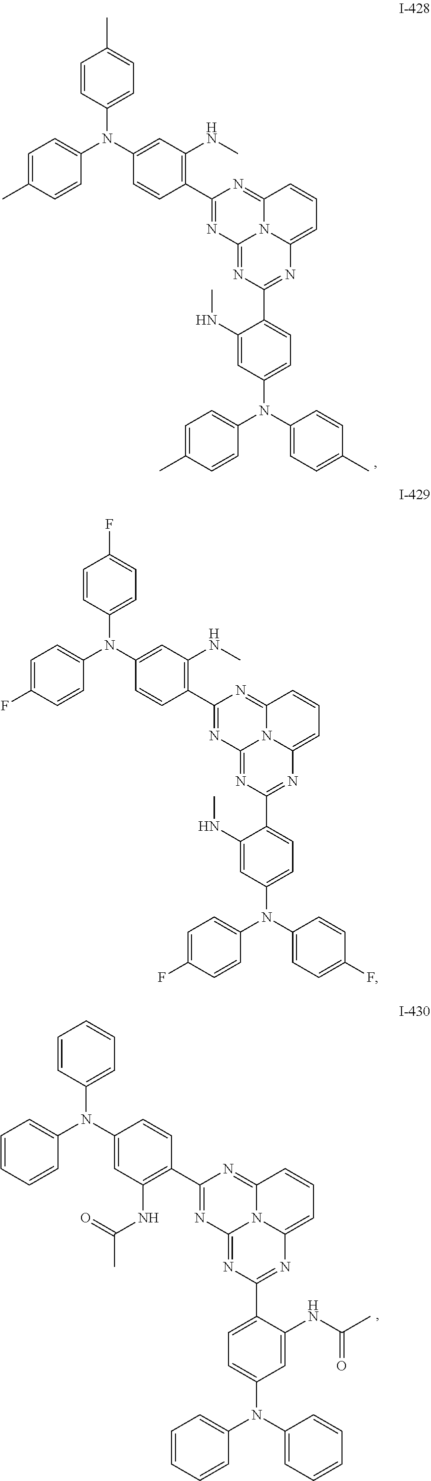

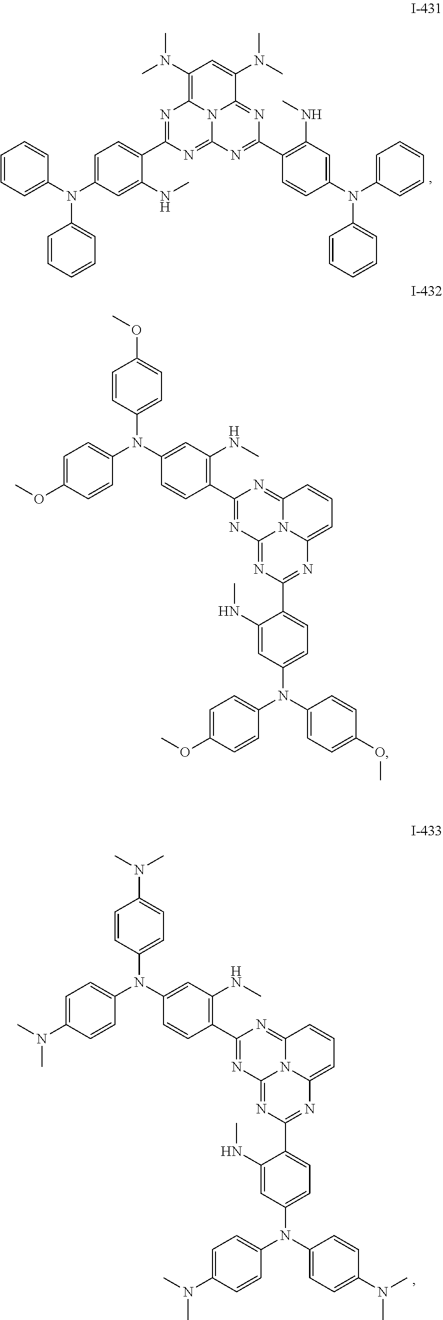

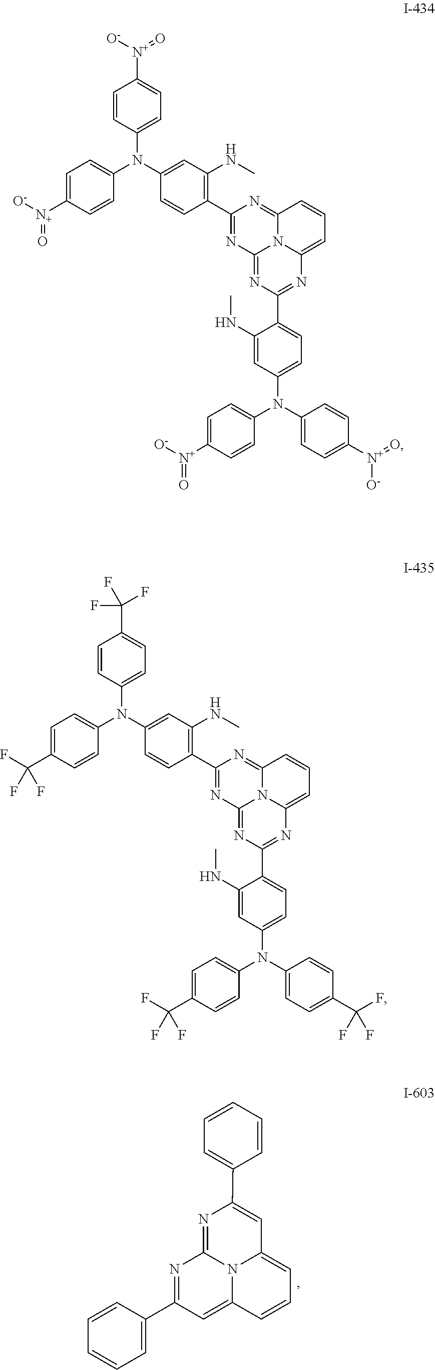 [SMILES: CNc1cc(N(c2ccc([N+](=O)[O-])cc2)c2ccc([N+](=O)[O-])cc2)ccc1C1=NC2=CC=CC3=NC(c4ccc(N(c5ccc([N+](=O)[O-])cc5)c5ccc([N+](=O)[O-])cc5)cc4NC)=NC(=N1)N23 ; CNc1cc(N(c2ccc(C(F)(F)F)cc2)c2ccc(C(F)(F)F)cc2)ccc1C1=NC2=CC=CC3=NC(c4ccc(N(c5ccc(C(F)(F)F)cc5)c5ccc(C(F)(F)F)cc5)cc4NC)=NC(=N1)N23 ; C1=CC2=CC(c3ccccc3)=NC3=NC(c4ccccc4)=CC(=C1)N23]

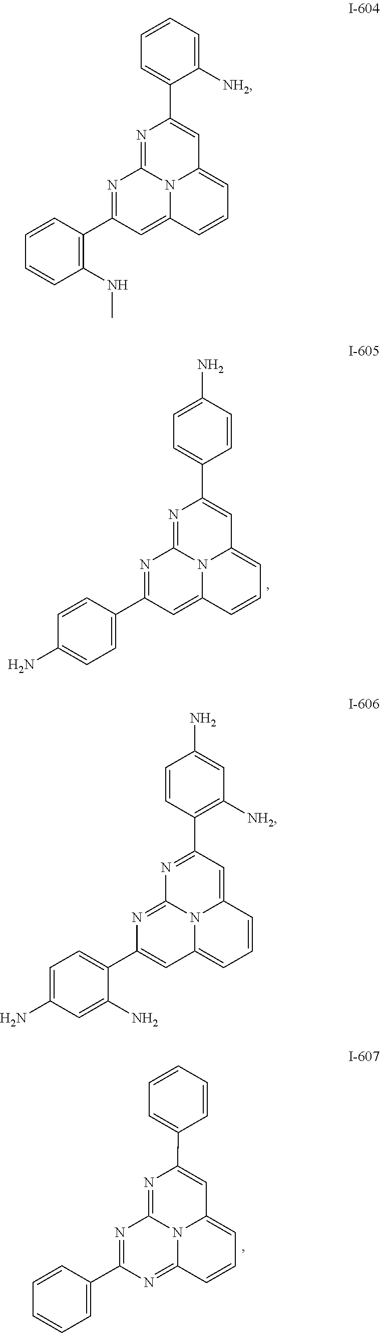 [SMILES: CNc1ccccc1C1=NC2=NC(c3ccccc3N)=CC3=CC=CC(=C1)N32 ; Nc1ccc(C2=CC3=CC=CC4=CC(c5ccc(N)cc5)=NC(=N2)N34)cc1 ; Nc1ccc(C2=CC3=CC=CC4=CC(c5ccc(N)cc5N)=NC(=N2)N34)c(N)c1 ; C1=CC2=NC(c3ccccc3)=NC3=NC(c4ccccc4)=CC(=C1)N23]

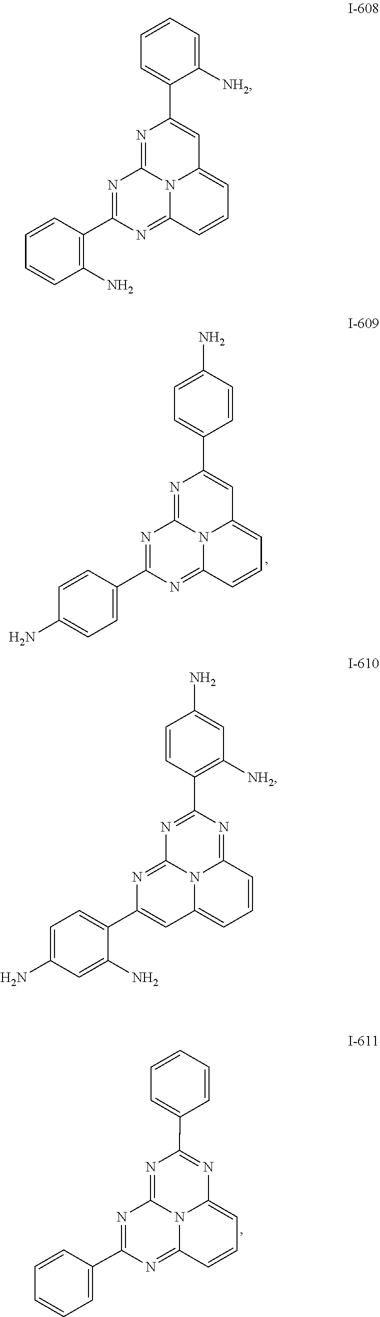 [SMILES: Nc1ccccc1C1=CC2=CC=CC3=NC(c4ccccc4N)=NC(=N1)N23 ; Nc1ccc(C2=CC3=CC=CC4=NC(c5ccc(N)cc5)=NC(=N2)N34)cc1 ; Nc1ccc(C2=CC3=CC=CC4=NC(c5ccc(N)cc5N)=NC(=N2)N34)c(N)c1 ; C1=CC2=NC(c3ccccc3)=NC3=NC(c4ccccc4)=NC(=C1)N23]

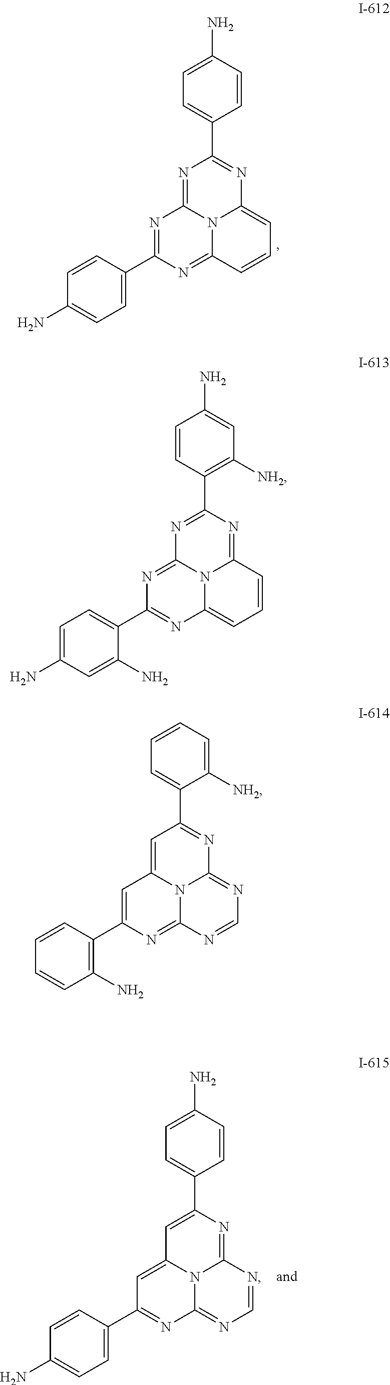 [SMILES: Nc1ccc(C2=NC3=CC=CC4=NC(c5ccc(N)cc5)=NC(=N2)N34)cc1 ; Nc1ccc(C2=NC3=CC=CC4=NC(c5ccc(N)cc5N)=NC(=N2)N34)c(N)c1 ; Nc1ccccc1C1=CC2=CC(c3ccccc3N)=NC3=NC=NC(=N1)N23 ; Nc1ccc(C2=CC3=CC(c4ccc(N)cc4)=NC4=NC=NC(=N2)N34)cc1]

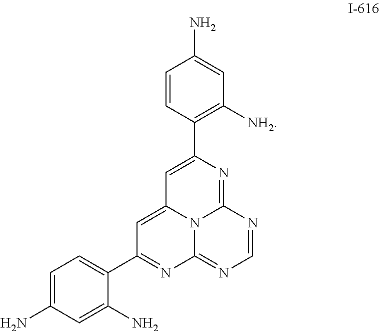 [SMILES: Nc1ccc(C2=CC3=CC(c4ccc(N)cc4N)=NC4=NC=NC(=N2)N34)c(N)c1]

14. The compound of any one of claims 1 and 9 to 12, wherein the compound has a structure of Formula I-a 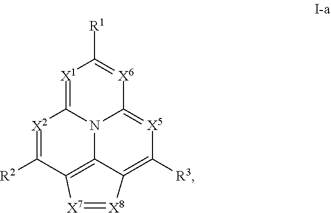
wherein
X⁷ is selected from N and CR¹¹; and
X⁸ is selected from N and CR¹².

15. The compound of claim 14, wherein R¹¹ and R¹² are each independently selected from H, NH₂, NH(alkyl), NH(aryl), and NH-heteroaryl.

16. The compound of claim 14, wherein R¹¹ and R¹² are H or NH₂.

17. The compound of any one of claims 14 to 16, wherein the compound is selected from

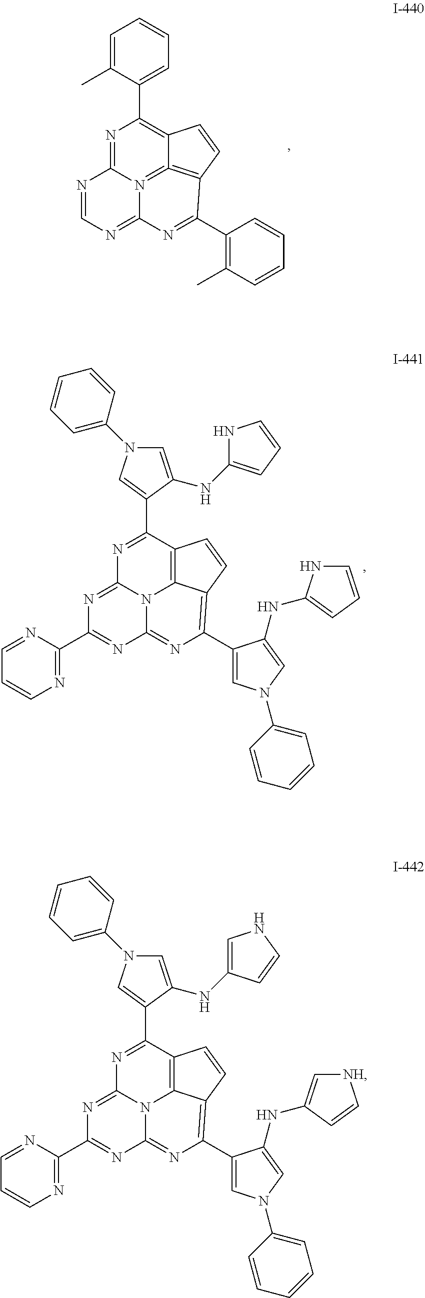

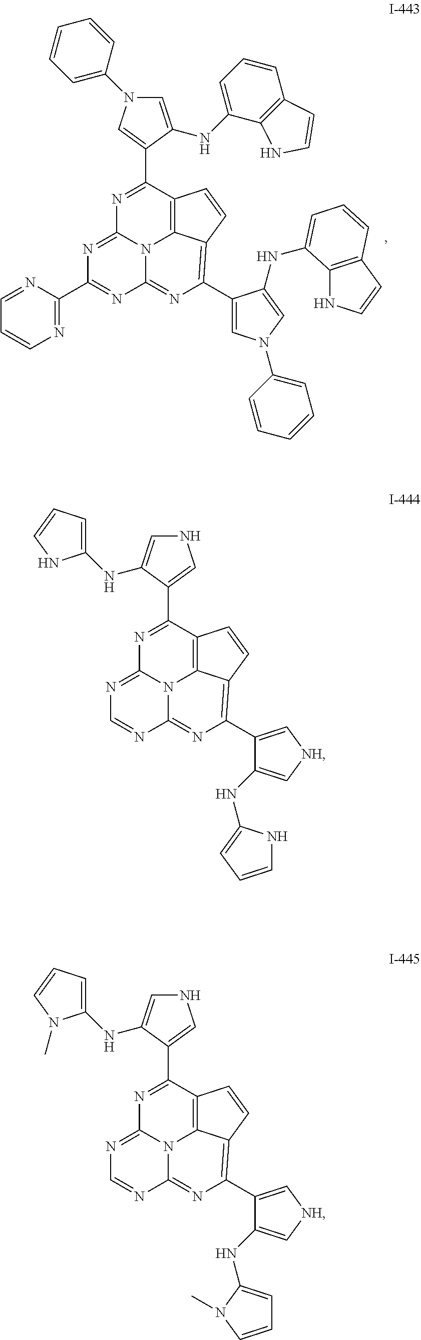 [SMILES: c1ccc(-n2cc(Nc3cccc4cc[nH]c34)c(-c3nc4nc(-c5ncccn5)nc5nc(-c6cn(-c7ccccc7)cc6Nc6cccc7cc[nH]c67)c6ccc3c6n45)c2)cc1 ; c1c[nH]c(Nc2c[nH]cc2-c2nc3ncnc4nc(-c5c[nH]cc5Nc5ccc[nH]5)c5ccc2c5n34)c1 ; Cn1cccc1Nc1c[nH]cc1-c1nc2ncnc3nc(-c4c[nH]cc4Nc4cccn4C)c4ccc1c4n23]

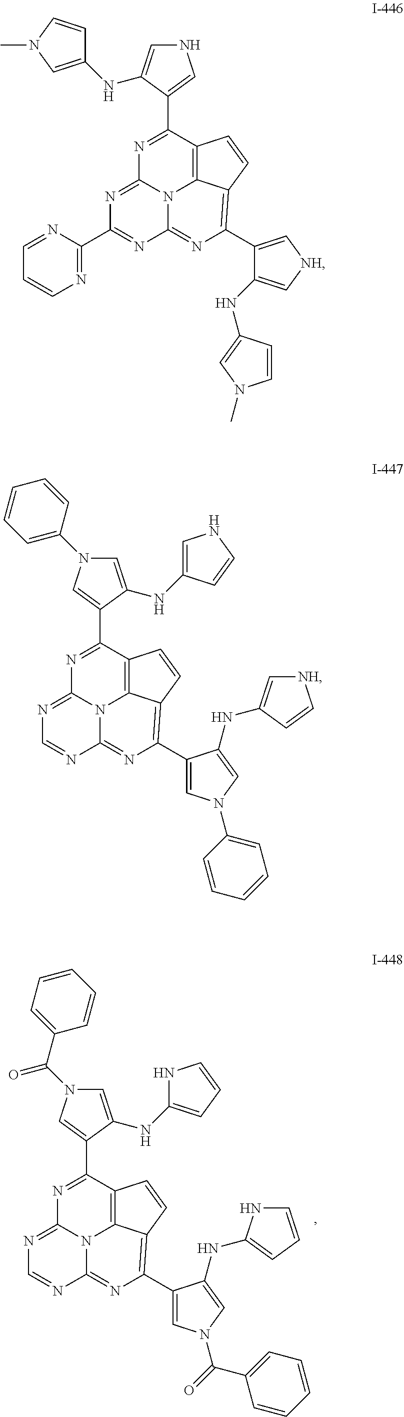 [SMILES: Cn1ccc(Nc2c[nH]cc2-c2nc3nc(-c4ncccn4)nc4nc(-c5c[nH]cc5Nc5ccn(C)c5)c5ccc2c5n34)c1 ; c1ccc(-n2cc(Nc3cc[nH]c3)c(-c3nc4ncnc5nc(-c6cn(-c7ccccc7)cc6Nc6cc[nH]c6)c6ccc3c6n45)c2)cc1 ; O=C(c1ccccc1)n1cc(Nc2ccc[nH]2)c(-c2nc3ncnc4nc(-c5cn(C(=O)c6ccccc6)cc5Nc5ccc[nH]5)c5ccc2c5n34)c1]

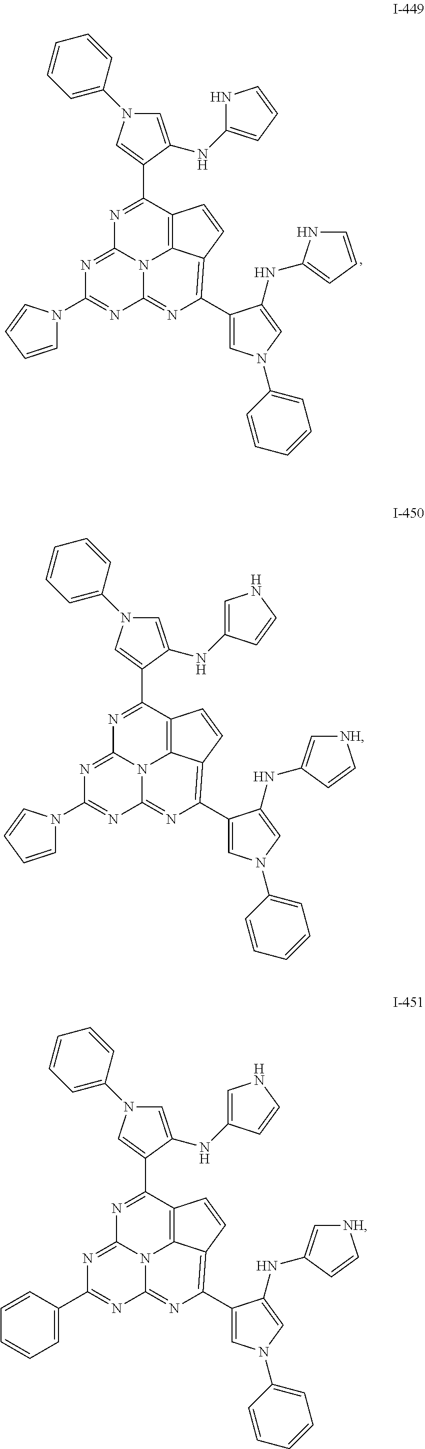 [SMILES: c1ccc(-n2cc(Nc3ccc[nH]3)c(-c3nc4nc(-n5cccc5)nc5nc(-c6cn(-c7ccccc7)cc6Nc6ccc[nH]6)c6ccc3c6n45)c2)cc1 ; c1ccc(-n2cc(Nc3cc[nH]c3)c(-c3nc4nc(-n5cccc5)nc5nc(-c6cn(-c7ccccc7)cc6Nc6cc[nH]c6)c6ccc3c6n45)c2)cc1 ; c1ccc(-c2nc3nc(-c4cn(-c5ccccc5)cc4Nc4cc[nH]c4)c4ccc5c(-c6cn(-c7ccccc7)cc6Nc6cc[nH]c6)nc(n2)n3c45)cc1]

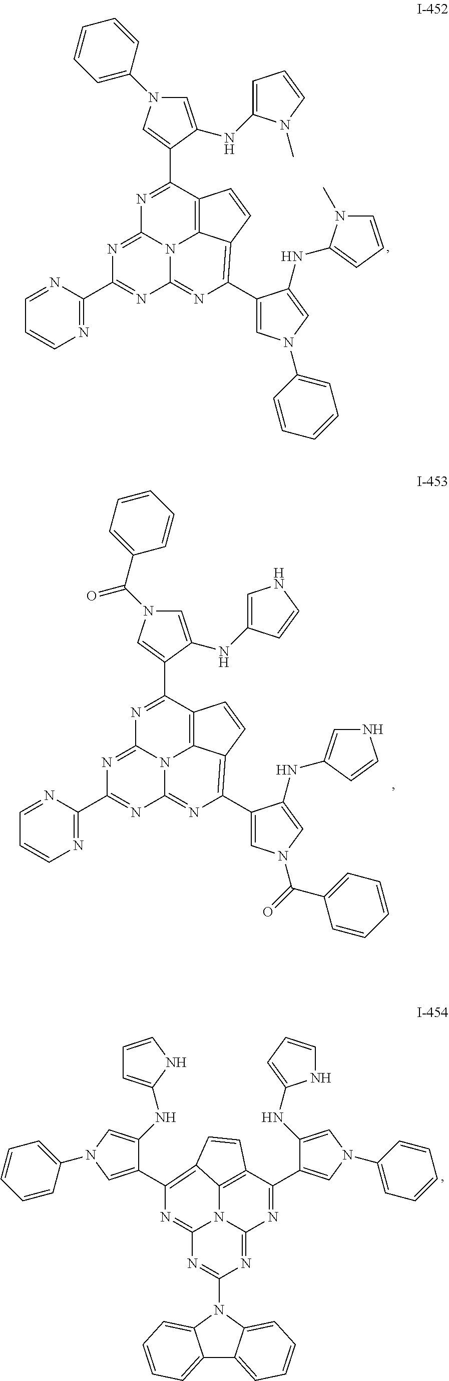 [SMILES: Cn1cccc1Nc1cn(-c2ccccc2)cc1-c1nc2nc(-c3ncccn3)nc3nc(-c4cn(-c5ccccc5)cc4Nc4cccn4C)c4ccc1c4n23 ; O=C(c1ccccc1)n1cc(Nc2cc[nH]c2)c(-c2nc3nc(-c4ncccn4)nc4nc(-c5cn(C(=O)c6ccccc6)cc5Nc5cc[nH]c5)c5ccc2c5n34)c1 ; c1ccc(-n2cc(Nc3ccc[nH]3)c(-c3nc4nc(-n5c6ccccc6c6ccccc65)nc5nc(-c6cn(-c7ccccc7)cc6Nc6ccc[nH]6)c6ccc3c6n45)c2)cc1]

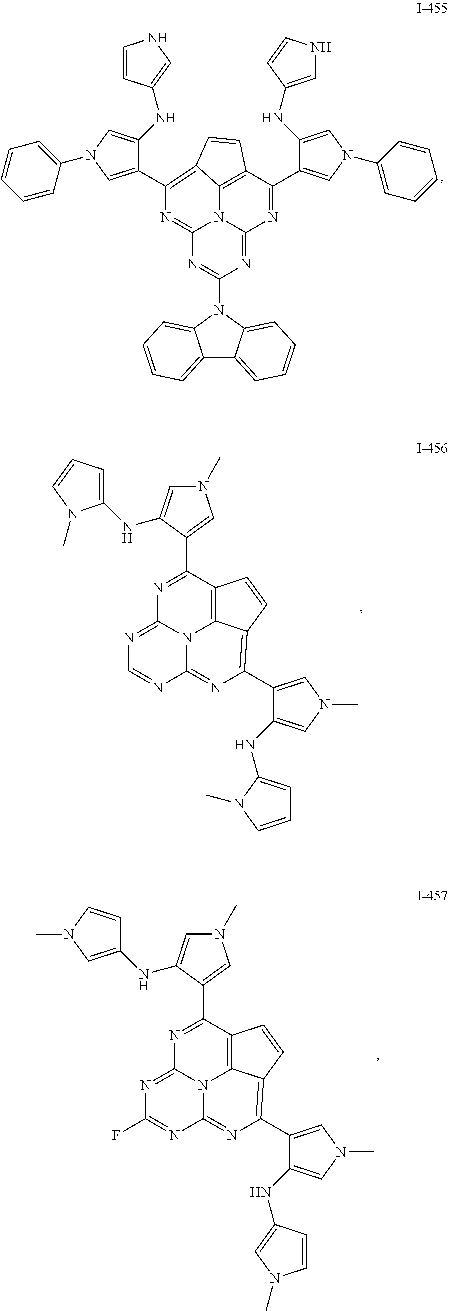

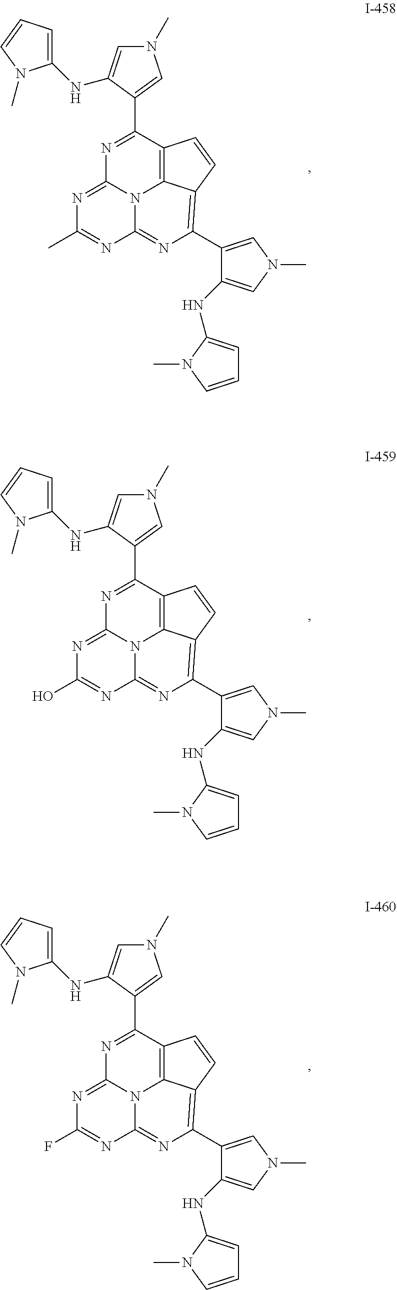 [SMILES: Cc1nc2nc(-c3cn(C)cc3Nc3cccn3C)c3ccc4c(-c5cn(C)cc5Nc5cccn5C)nc(n1)n2c34 ; Cn1cc(Nc2cccn2C)c(-c2nc3nc(O)nc4nc(-c5cn(C)cc5Nc5cccn5C)c5ccc2c5n34)c1 ; Cn1cc(Nc2cccn2C)c(-c2nc3nc(F)nc4nc(-c5cn(C)cc5Nc5cccn5C)c5ccc2c5n34)c1]

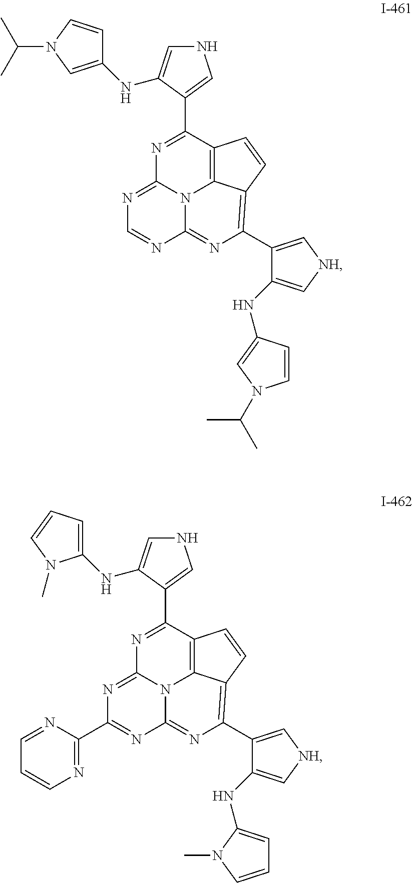 [SMILES: CC(C)n1ccc(Nc2c[nH]cc2-c2nc3ncnc4nc(-c5c[nH]cc5Nc5ccn(C(C)C)c5)c5ccc2c5n34)c1 ; Cn1cccc1Nc1c[nH]cc1-c1nc2nc(-c3ncccn3)nc3nc(-c4c[nH]cc4Nc4cccn4C)c4ccc1c4n23]

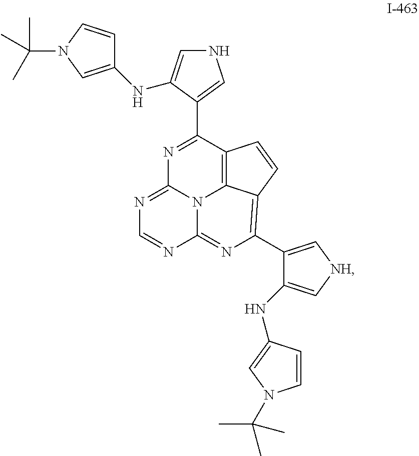 [SMILES: CC(C)(C)n1ccc(Nc2c[nH]cc2-c2nc3ncnc4nc(-c5c[nH]cc5Nc5ccn(C(C)(C)C)c5)c5ccc2c5n34)c1]

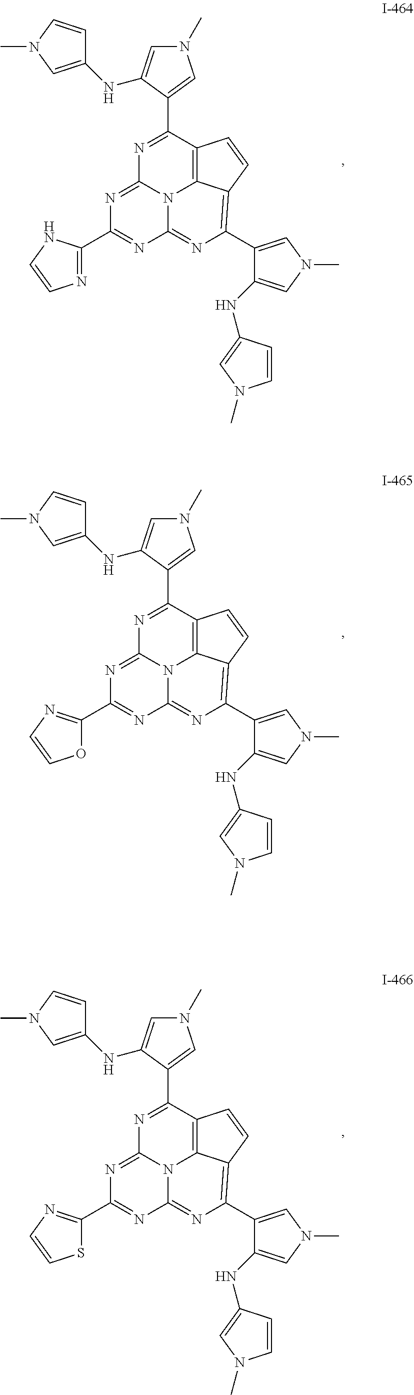

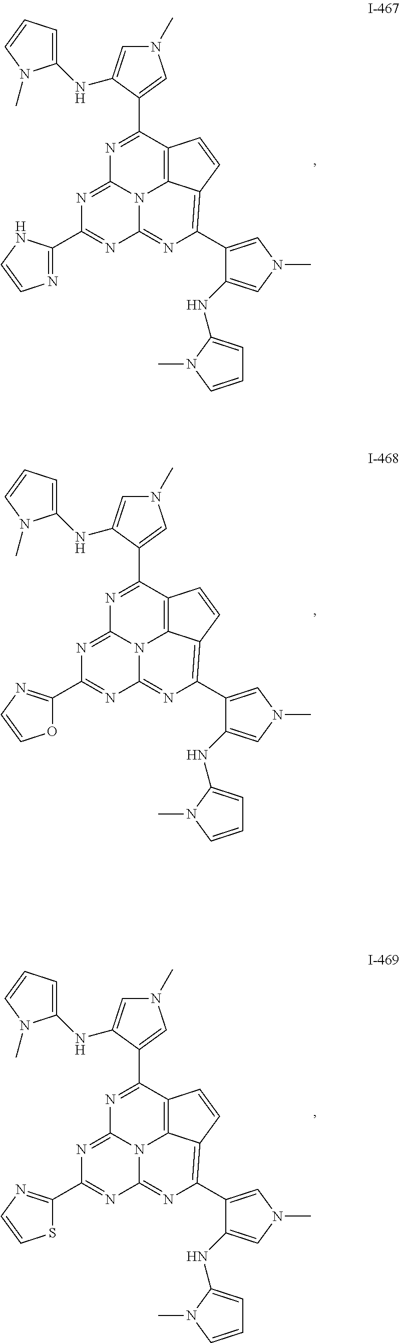 [SMILES: Cn1cc(Nc2cccn2C)c(-c2nc3nc(-c4ncc[nH]4)nc4nc(-c5cn(C)cc5Nc5cccn5C)c5ccc2c5n34)c1 ; Cn1cc(Nc2cccn2C)c(-c2nc3nc(-c4ncco4)nc4nc(-c5cn(C)cc5Nc5cccn5C)c5ccc2c5n34)c1 ; Cn1cc(Nc2cccn2C)c(-c2nc3nc(-c4nccs4)nc4nc(-c5cn(C)cc5Nc5cccn5C)c5ccc2c5n34)c1]

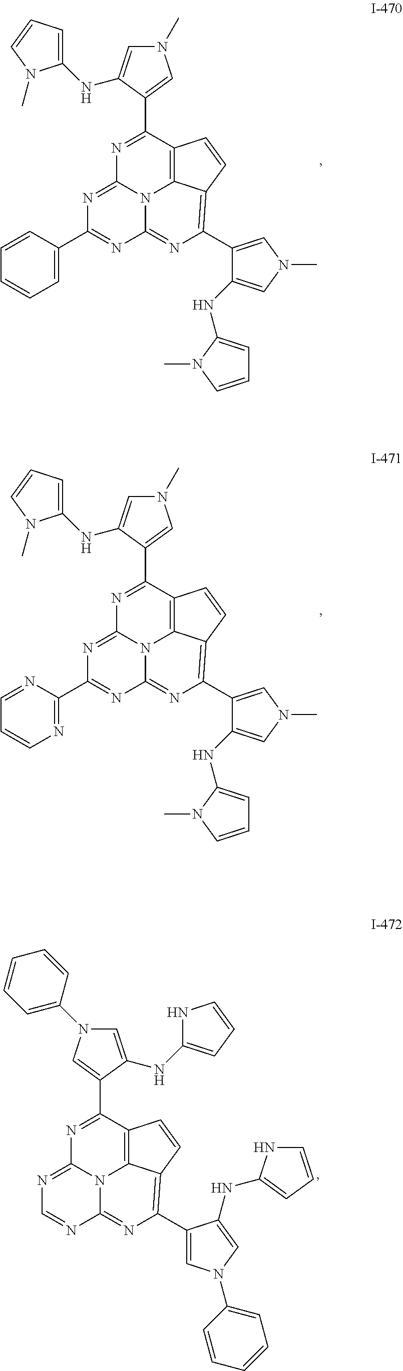 [SMILES: Cn1cc(Nc2cccn2C)c(-c2nc3nc(-c4ccccc4)nc4nc(-c5cn(C)cc5Nc5cccn5C)c5ccc2c5n34)c1 ; Cn1cc(Nc2cccn2C)c(-c2nc3nc(-c4ncccn4)nc4nc(-c5cn(C)cc5Nc5cccn5C)c5ccc2c5n34)c1 ; c1ccc(-n2cc(Nc3ccc[nH]3)c(-c3nc4ncnc5nc(-c6cn(-c7ccccc7)cc6Nc6ccc[nH]6)c6ccc3c6n45)c2)cc1]

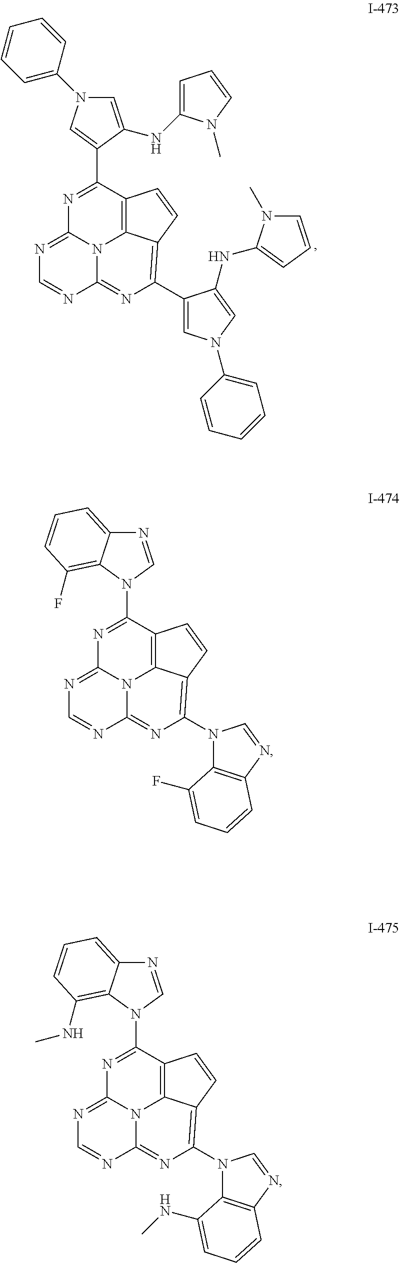 [SMILES: Cn1cccc1Nc1cn(-c2ccccc2)cc1-c1nc2ncnc3nc(-c4cn(-c5ccccc5)cc4Nc4cccn4C)c4ccc1c4n23 ; Fc1cccc2ncn(-c3nc4ncnc5nc(-n6cnc7cccc(F)c76)c6ccc3c6n45)c12 ; CNc1cccc2ncn(-c3nc4ncnc5nc(-n6cnc7cccc(NC)c76)c6ccc3c6n45)c12]

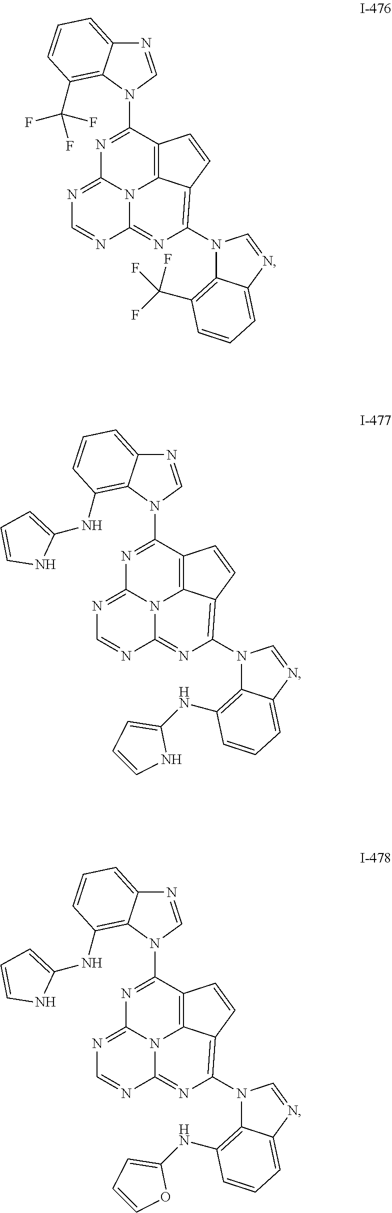 [SMILES: FC(F)(F)c1cccc2ncn(-c3nc4ncnc5nc(-n6cnc7cccc(C(F)(F)F)c76)c6ccc3c6n45)c12 ; c1c[nH]c(Nc2cccc3ncn(-c4nc5ncnc6nc(-n7cnc8cccc(Nc9ccc[nH]9)c87)c7ccc4c7n56)c23)c1 ; c1c[nH]c(Nc2cccc3ncn(-c4nc5ncnc6nc(-n7cnc8cccc(Nc9ccco9)c87)c7ccc4c7n56)c23)c1]

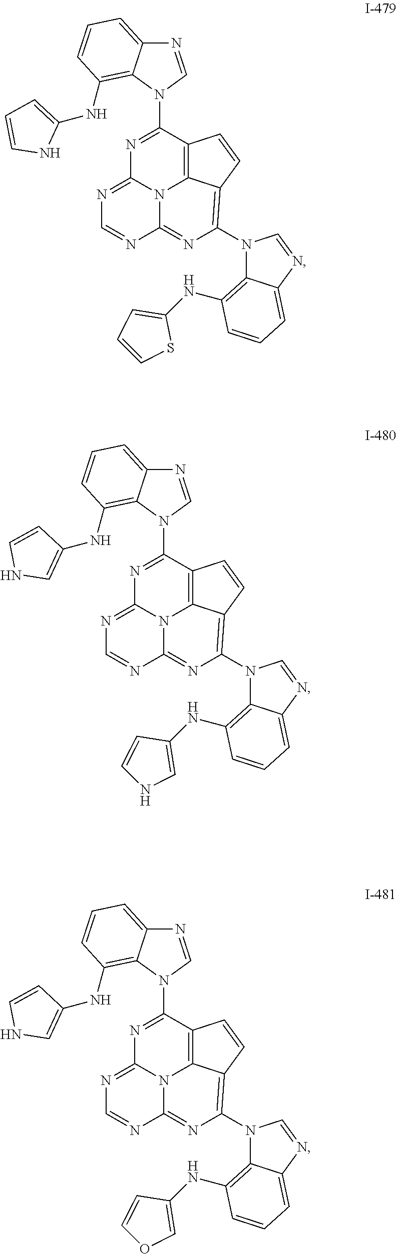 [SMILES: c1c[nH]c(Nc2cccc3ncn(-c4nc5ncnc6nc(-n7cnc8cccc(Nc9cccs9)c87)c7ccc4c7n56)c23)c1 ; c1cc(Nc2cc[nH]c2)c2c(c1)ncn2-c1nc2ncnc3nc(-n4cnc5cccc(Nc6cc[nH]c6)c54)c4ccc1c4n23 ; c1cc(Nc2cc[nH]c2)c2c(c1)ncn2-c1nc2ncnc3nc(-n4cnc5cccc(Nc6ccoc6)c54)c4ccc1c4n23]

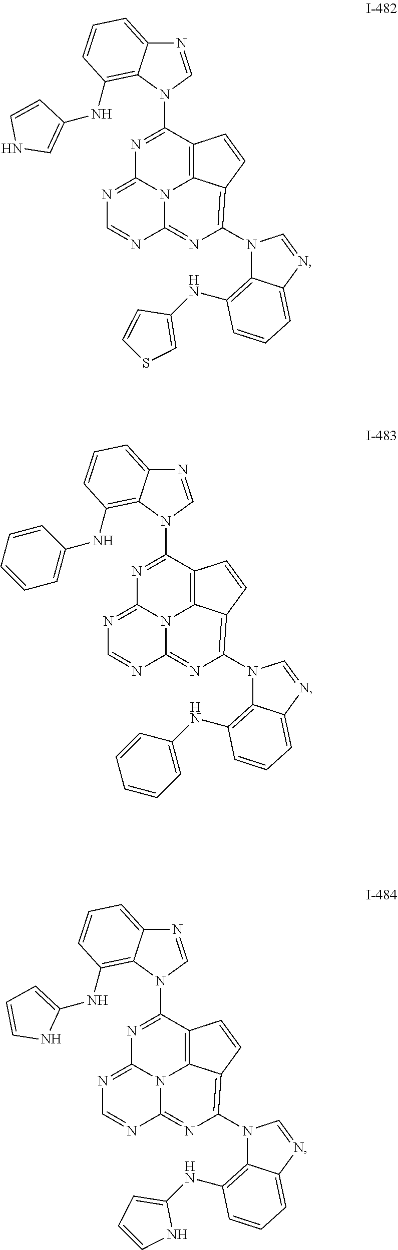 [SMILES: c1cc(Nc2cc[nH]c2)c2c(c1)ncn2-c1nc2ncnc3nc(-n4cnc5cccc(Nc6ccsc6)c54)c4ccc1c4n23 ; c1ccc(Nc2cccc3ncn(-c4nc5ncnc6nc(-n7cnc8cccc(Nc9ccccc9)c87)c7ccc4c7n56)c23)cc1 ; c1c[nH]c(Nc2cccc3ncn(-c4nc5ncnc6nc(-n7cnc8cccc(Nc9ccc[nH]9)c87)c7ccc4c7n56)c23)c1]

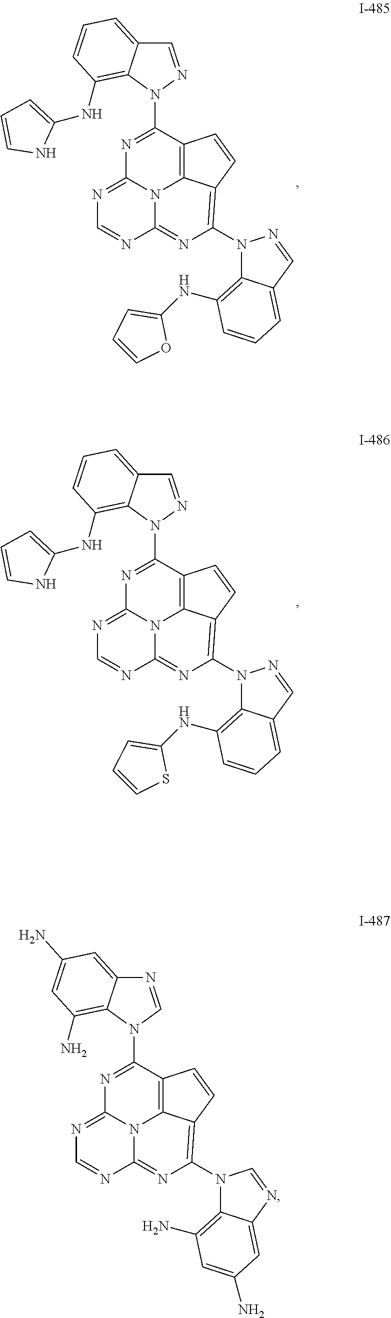 [SMILES: c1c[nH]c(Nc2cccc3cnn(-c4nc5ncnc6nc(-n7ncc8cccc(Nc9ccco9)c87)c7ccc4c7n56)c23)c1 ; c1c[nH]c(Nc2cccc3cnn(-c4nc5ncnc6nc(-n7ncc8cccc(Nc9cccs9)c87)c7ccc4c7n56)c23)c1 ; Nc1cc(N)c2c(c1)ncn2-c1nc2ncnc3nc(-n4cnc5cc(N)cc(N)c54)c4ccc1c4n23]

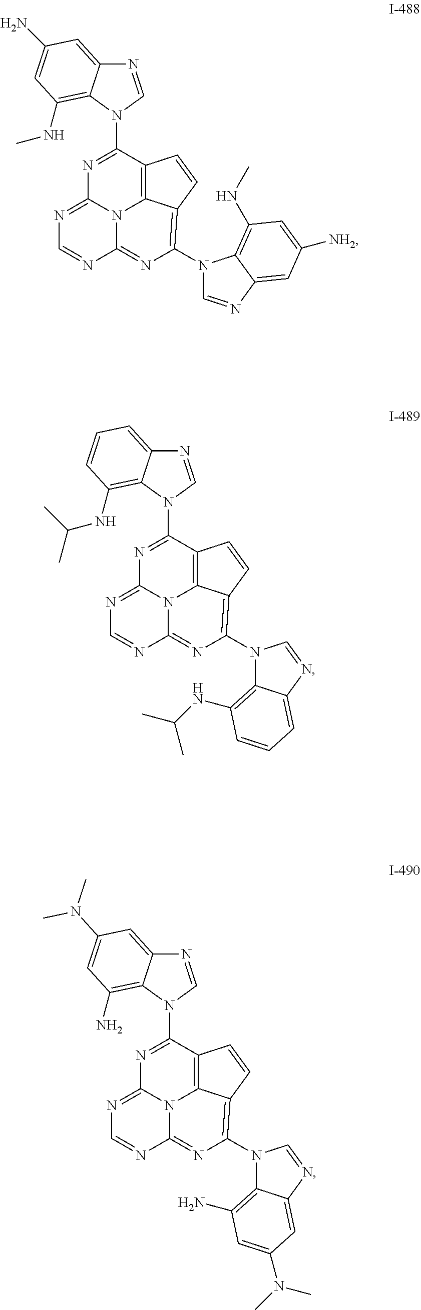

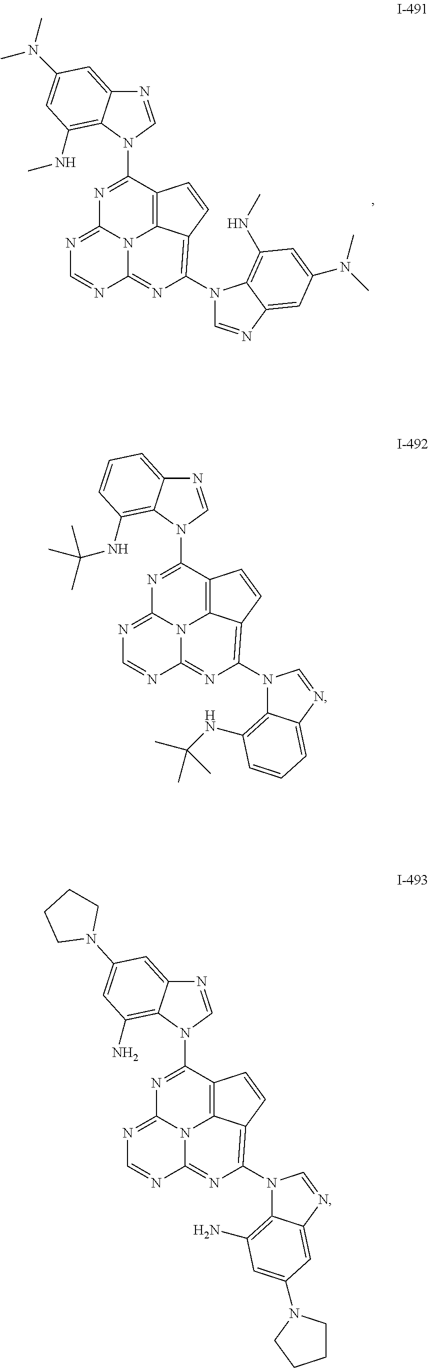

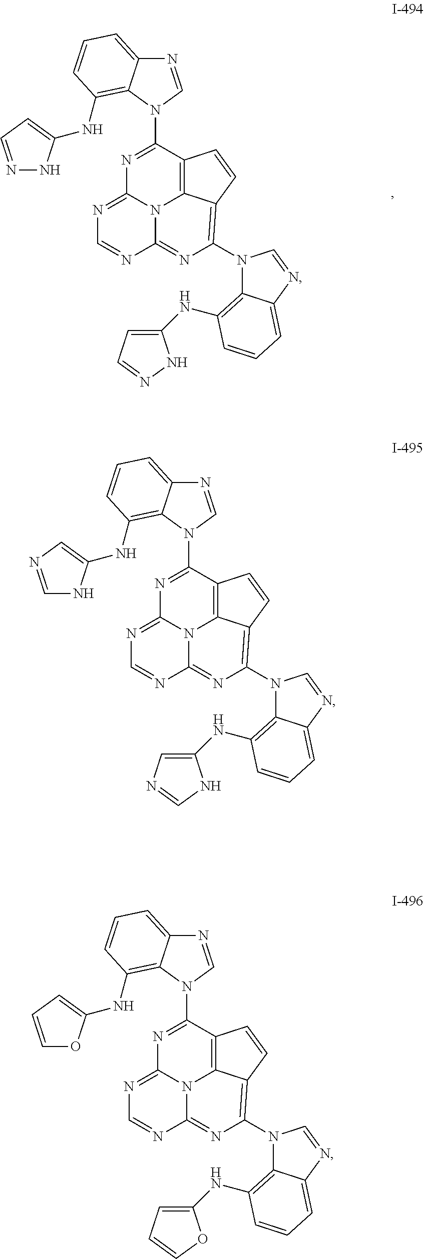 [SMILES: c1cc(Nc2ccn[nH]2)c2c(c1)ncn2-c1nc2ncnc3nc(-n4cnc5cccc(Nc6ccn[nH]6)c54)c4ccc1c4n23 ; c1cc(Nc2cnc[nH]2)c2c(c1)ncn2-c1nc2ncnc3nc(-n4cnc5cccc(Nc6cnc[nH]6)c54)c4ccc1c4n23 ; c1coc(Nc2cccc3ncn(-c4nc5ncnc6nc(-n7cnc8cccc(Nc9ccco9)c87)c7ccc4c7n56)c23)c1]

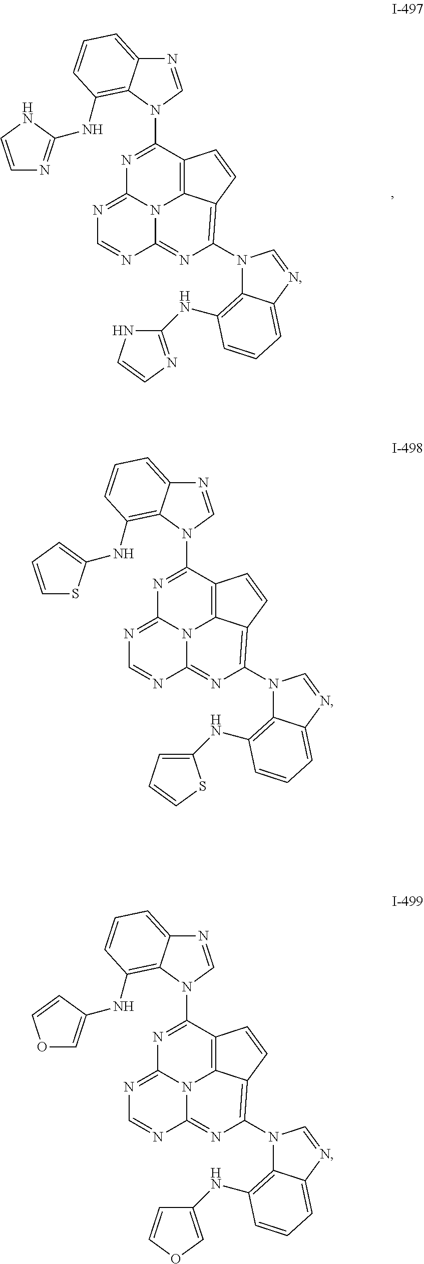 [SMILES: c1cc(Nc2ncc[nH]2)c2c(c1)ncn2-c1nc2ncnc3nc(-n4cnc5cccc(Nc6ncc[nH]6)c54)c4ccc1c4n23 ; c1csc(Nc2cccc3ncn(-c4nc5ncnc6nc(-n7cnc8cccc(Nc9cccs9)c87)c7ccc4c7n56)c23)c1 ; c1cc(Nc2ccoc2)c2c(c1)ncn2-c1nc2ncnc3nc(-n4cnc5cccc(Nc6ccoc6)c54)c4ccc1c4n23]

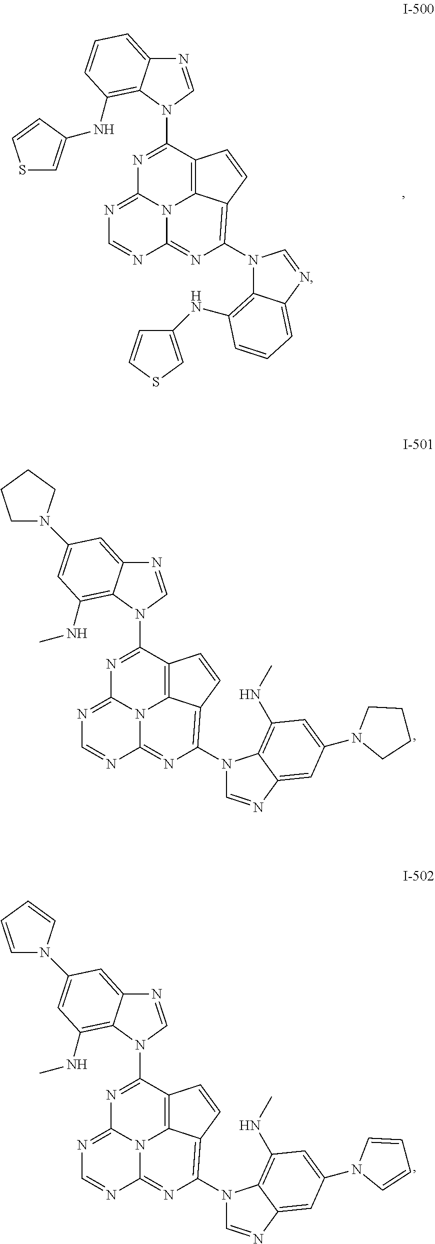 [SMILES: c1cc(Nc2ccsc2)c2c(c1)ncn2-c1nc2ncnc3nc(-n4cnc5cccc(Nc6ccsc6)c54)c4ccc1c4n23 ; CNc1cc(N2CCCC2)cc2ncn(-c3nc4ncnc5nc(-n6cnc7cc(N8CCCC8)cc(NC)c76)c6ccc3c6n45)c12 ; CNc1cc(-n2cccc2)cc2ncn(-c3nc4ncnc5nc(-n6cnc7cc(-n8cccc8)cc(NC)c76)c6ccc3c6n45)c12]

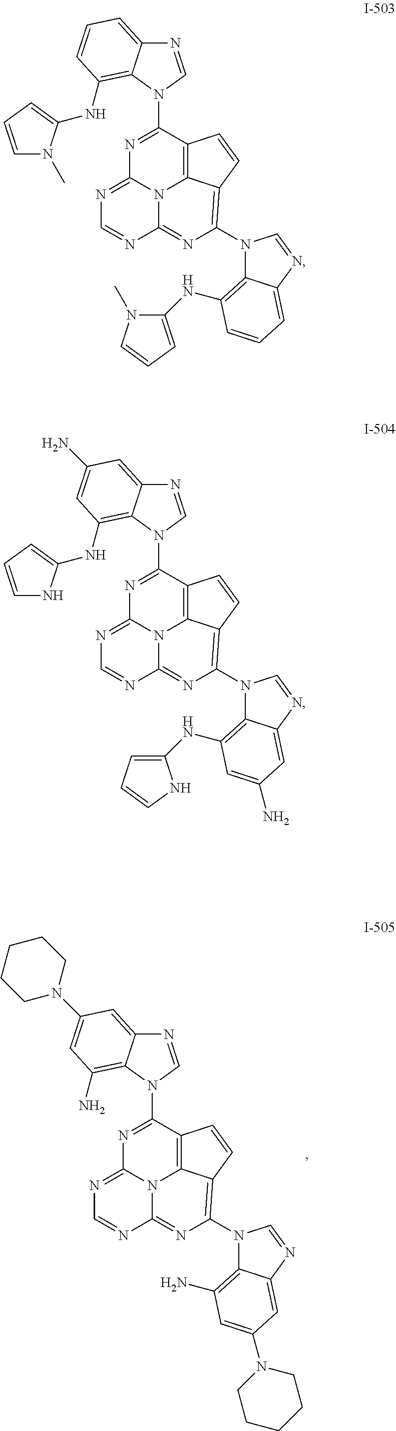 [SMILES: Cn1cccc1Nc1cccc2ncn(-c3nc4ncnc5nc(-n6cnc7cccc(Nc8cccn8C)c76)c6ccc3c6n45)c12 ; Nc1cc(Nc2ccc[nH]2)c2c(c1)ncn2-c1nc2ncnc3nc(-n4cnc5cc(N)cc(Nc6ccc[nH]6)c54)c4ccc1c4n23 ; Nc1cc(N2CCCCC2)cc2ncn(-c3nc4ncnc5nc(-n6cnc7cc(N8CCCCC8)cc(N)c76)c6ccc3c6n45)c12]

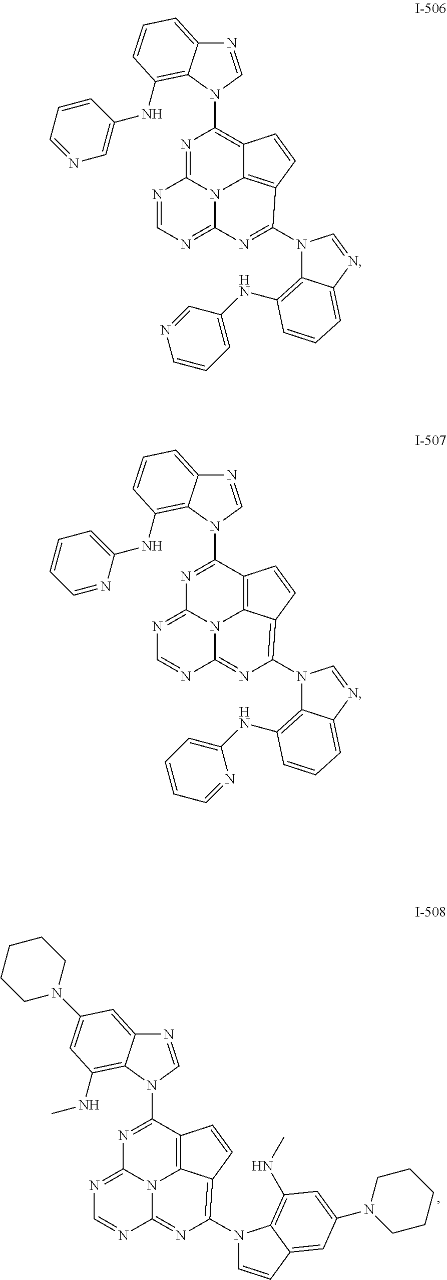 [SMILES: c1cncc(Nc2cccc3ncn(-c4nc5ncnc6nc(-n7cnc8cccc(Nc9cccnc9)c87)c7ccc4c7n56)c23)c1 ; c1ccc(Nc2cccc3ncn(-c4nc5ncnc6nc(-n7cnc8cccc(Nc9ccccn9)c87)c7ccc4c7n56)c23)nc1 ; CNc1cc(N2CCCCC2)cc2ccn(-c3nc4ncnc5nc(-n6cnc7cc(N8CCCCC8)cc(NC)c76)c6ccc3c6n45)c12]

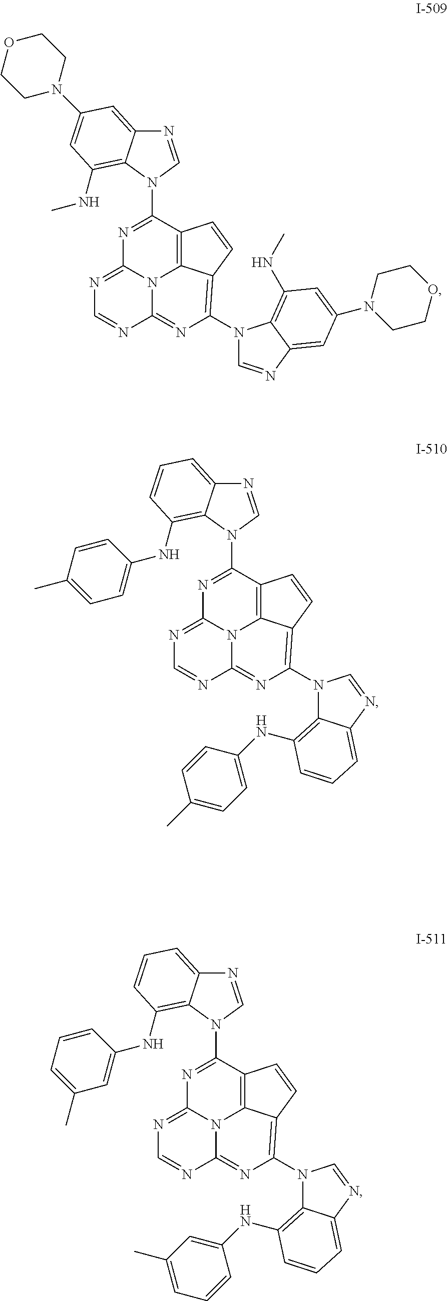 [SMILES: CNc1cc(N2CCOCC2)cc2ncn(-c3nc4ncnc5nc(-n6cnc7cc(N8CCOCC8)cc(NC)c76)c6ccc3c6n45)c12 ; Cc1ccc(Nc2cccc3ncn(-c4nc5ncnc6nc(-n7cnc8cccc(Nc9ccc(C)cc9)c87)c7ccc4c7n56)c23)cc1 ; Cc1cccc(Nc2cccc3ncn(-c4nc5ncnc6nc(-n7cnc8cccc(Nc9cccc(C)c9)c87)c7ccc4c7n56)c23)c1]

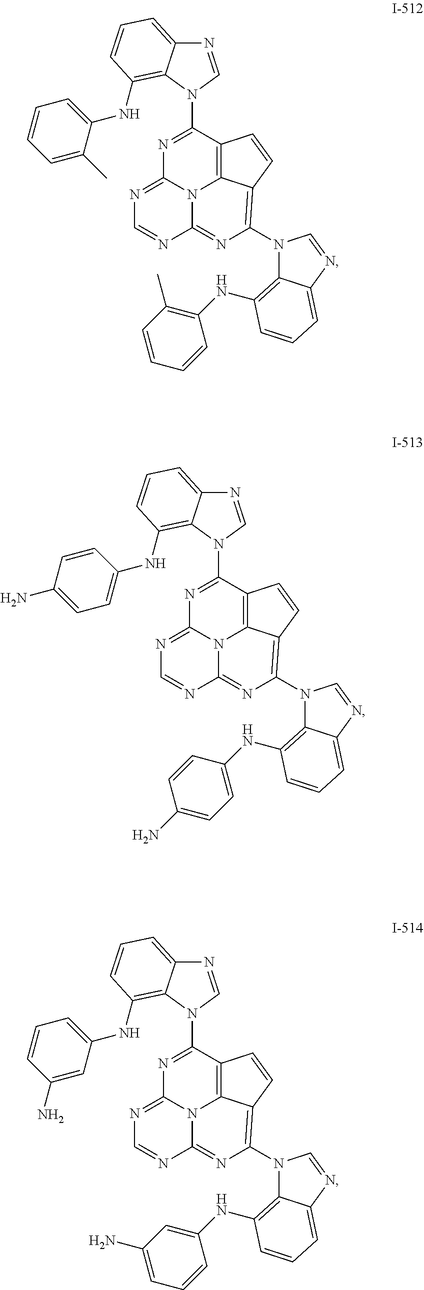 [SMILES: Cc1ccccc1Nc1cccc2ncn(-c3nc4ncnc5nc(-n6cnc7cccc(Nc8ccccc8C)c76)c6ccc3c6n45)c12 ; Nc1ccc(Nc2cccc3ncn(-c4nc5ncnc6nc(-n7cnc8cccc(Nc9ccc(N)cc9)c87)c7ccc4c7n56)c23)cc1 ; Nc1cccc(Nc2cccc3ncn(-c4nc5ncnc6nc(-n7cnc8cccc(Nc9cccc(N)c9)c87)c7ccc4c7n56)c23)c1]

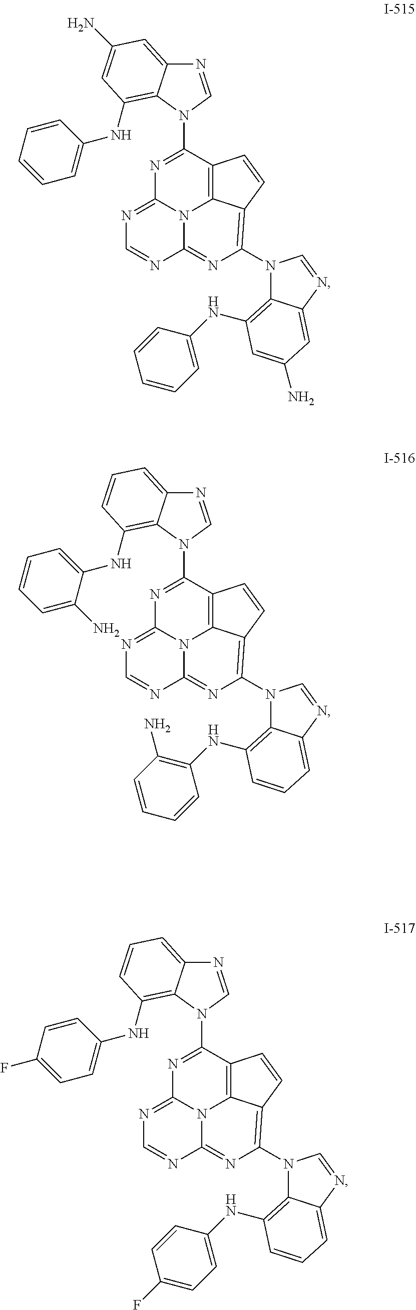 [SMILES: Nc1cc(Nc2ccccc2)c2c(c1)ncn2-c1nc2ncnc3nc(-n4cnc5cc(N)cc(Nc6ccccc6)c54)c4ccc1c4n23 ; Nc1ccccc1Nc1cccc2ncn(-c3nc4ncnc5nc(-n6cnc7cccc(Nc8ccccc8N)c76)c6ccc3c6n45)c12 ; Fc1ccc(Nc2cccc3ncn(-c4nc5ncnc6nc(-n7cnc8cccc(Nc9ccc(F)cc9)c87)c7ccc4c7n56)c23)cc1]

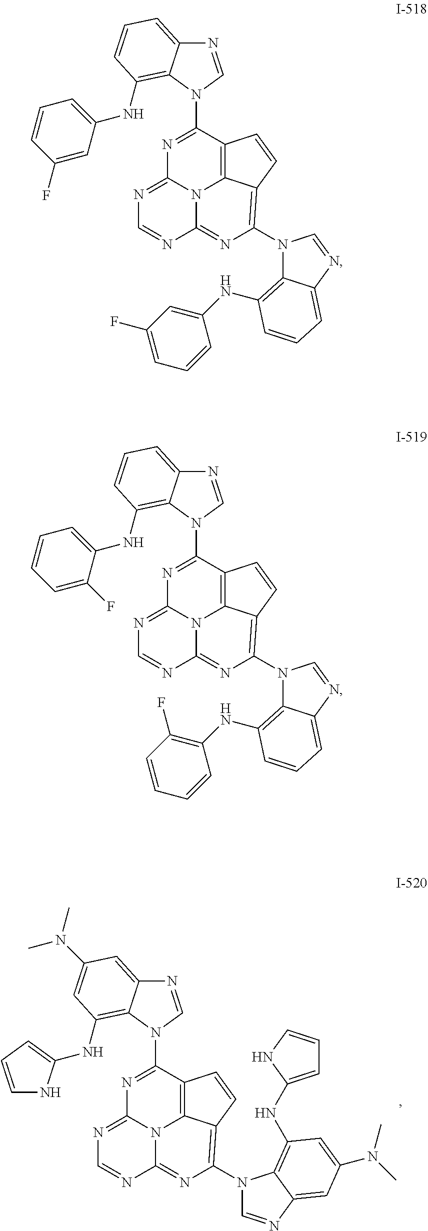 [SMILES: Fc1cccc(Nc2cccc3ncn(-c4nc5ncnc6nc(-n7cnc8cccc(Nc9cccc(F)c9)c87)c7ccc4c7n56)c23)c1 ; Fc1ccccc1Nc1cccc2ncn(-c3nc4ncnc5nc(-n6cnc7cccc(Nc8ccccc8F)c76)c6ccc3c6n45)c12 ; CN(C)c1cc(Nc2ccc[nH]2)c2c(c1)ncn2-c1nc2ncnc3nc(-n4cnc5cc(N(C)C)cc(Nc6ccc[nH]6)c54)c4ccc1c4n23]

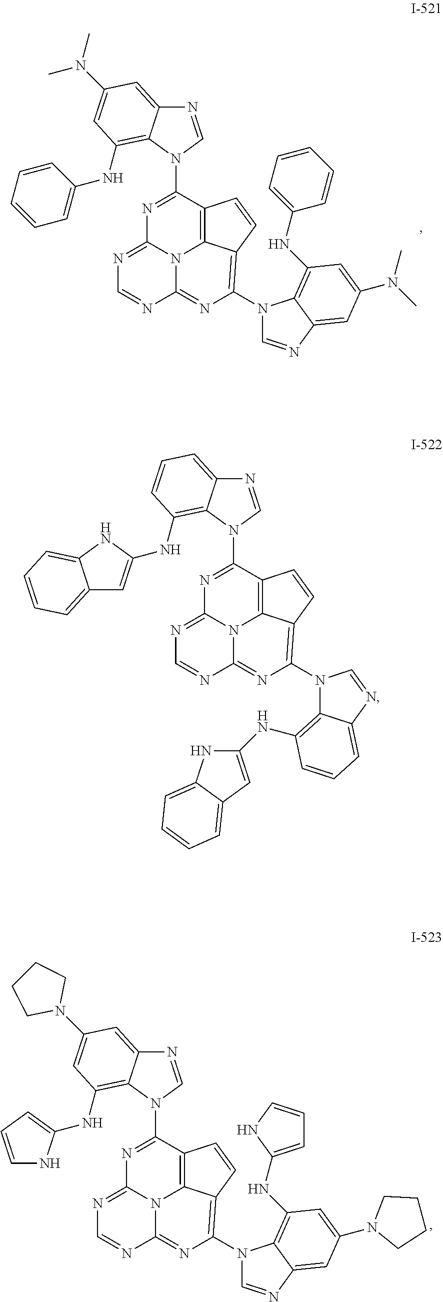 [SMILES: CN(C)c1cc(Nc2ccccc2)c2c(c1)ncn2-c1nc2ncnc3nc(-n4cnc5cc(N(C)C)cc(Nc6ccccc6)c54)c4ccc1c4n23 ; c1ccc2[nH]c(Nc3cccc4ncn(-c5nc6ncnc7nc(-n8cnc9cccc(Nc%10cc%11ccccc%11[nH]%10)c98)c8ccc5c8n67)c34)cc2c1 ; c1c[nH]c(Nc2cc(N3CCCC3)cc3ncn(-c4nc5ncnc6nc(-n7cnc8cc(N9CCCC9)cc(Nc9ccc[nH]9)c87)c7ccc4c7n56)c23)c1]

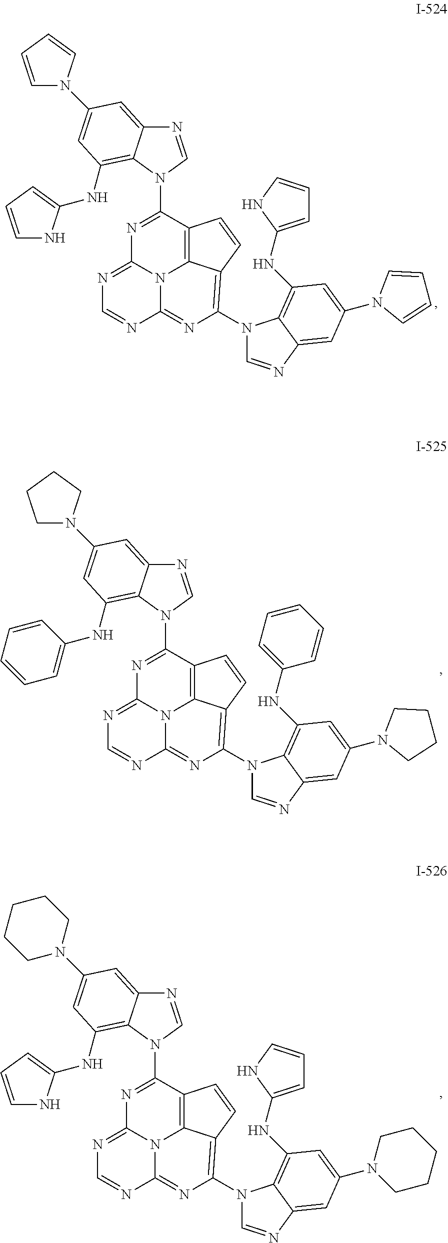 [SMILES: c1c[nH]c(Nc2cc(-n3cccc3)cc3ncn(-c4nc5ncnc6nc(-n7cnc8cc(-n9cccc9)cc(Nc9ccc[nH]9)c87)c7ccc4c7n56)c23)c1 ; c1ccc(Nc2cc(N3CCCC3)cc3ncn(-c4nc5ncnc6nc(-n7cnc8cc(N9CCCC9)cc(Nc9ccccc9)c87)c7ccc4c7n56)c23)cc1 ; c1c[nH]c(Nc2cc(N3CCCCC3)cc3ncn(-c4nc5ncnc6nc(-n7cnc8cc(N9CCCCC9)cc(Nc9ccc[nH]9)c87)c7ccc4c7n56)c23)c1]

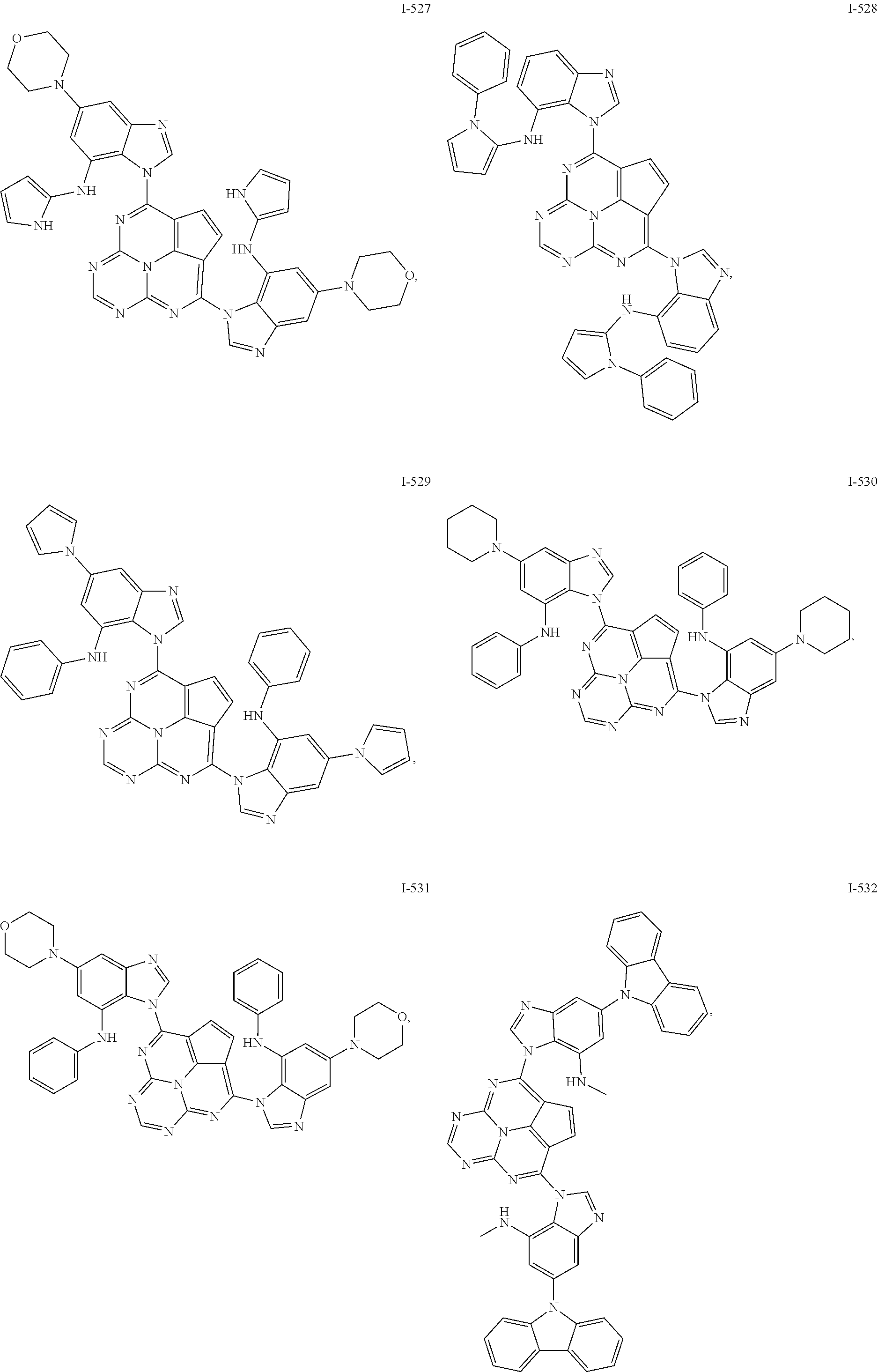

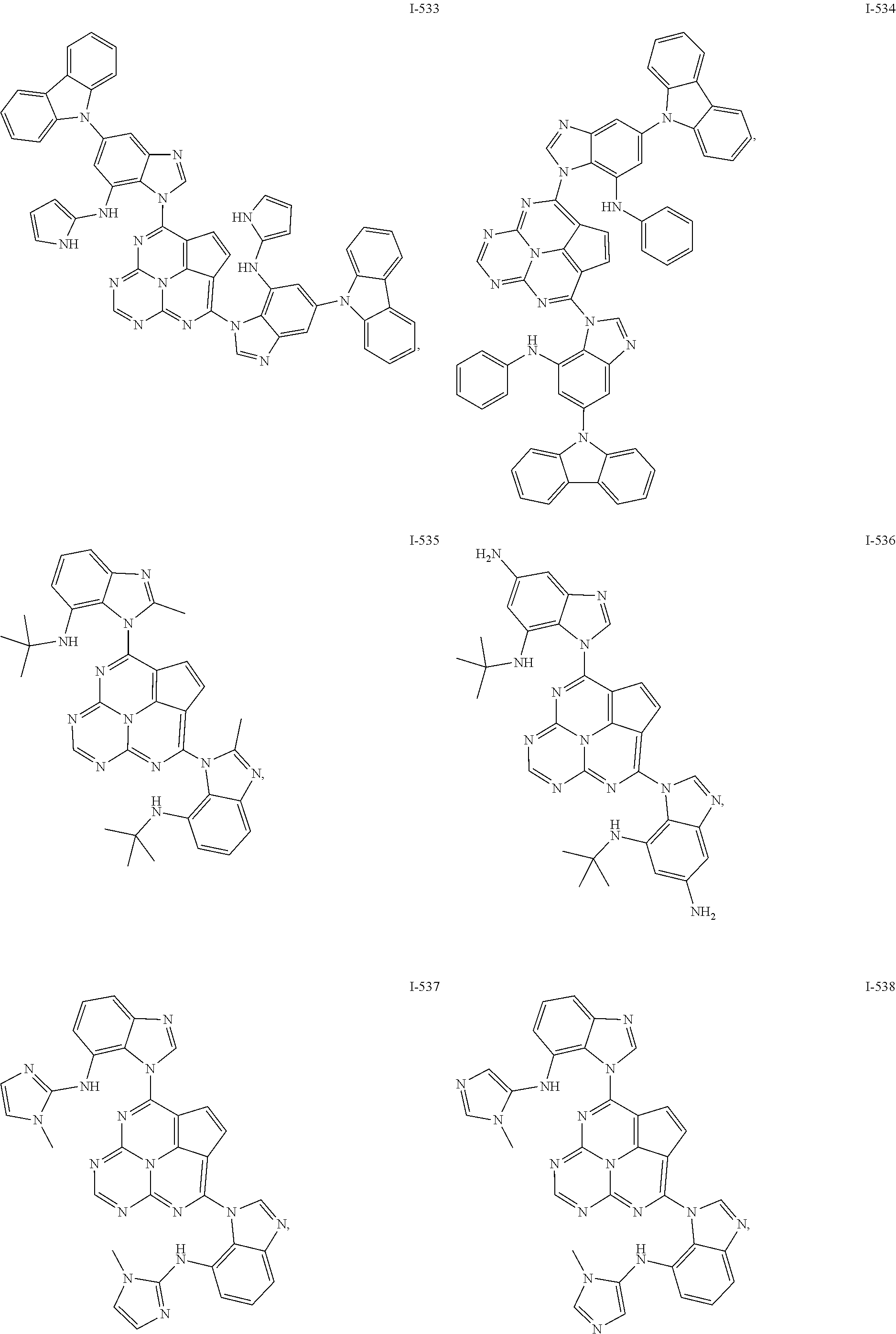

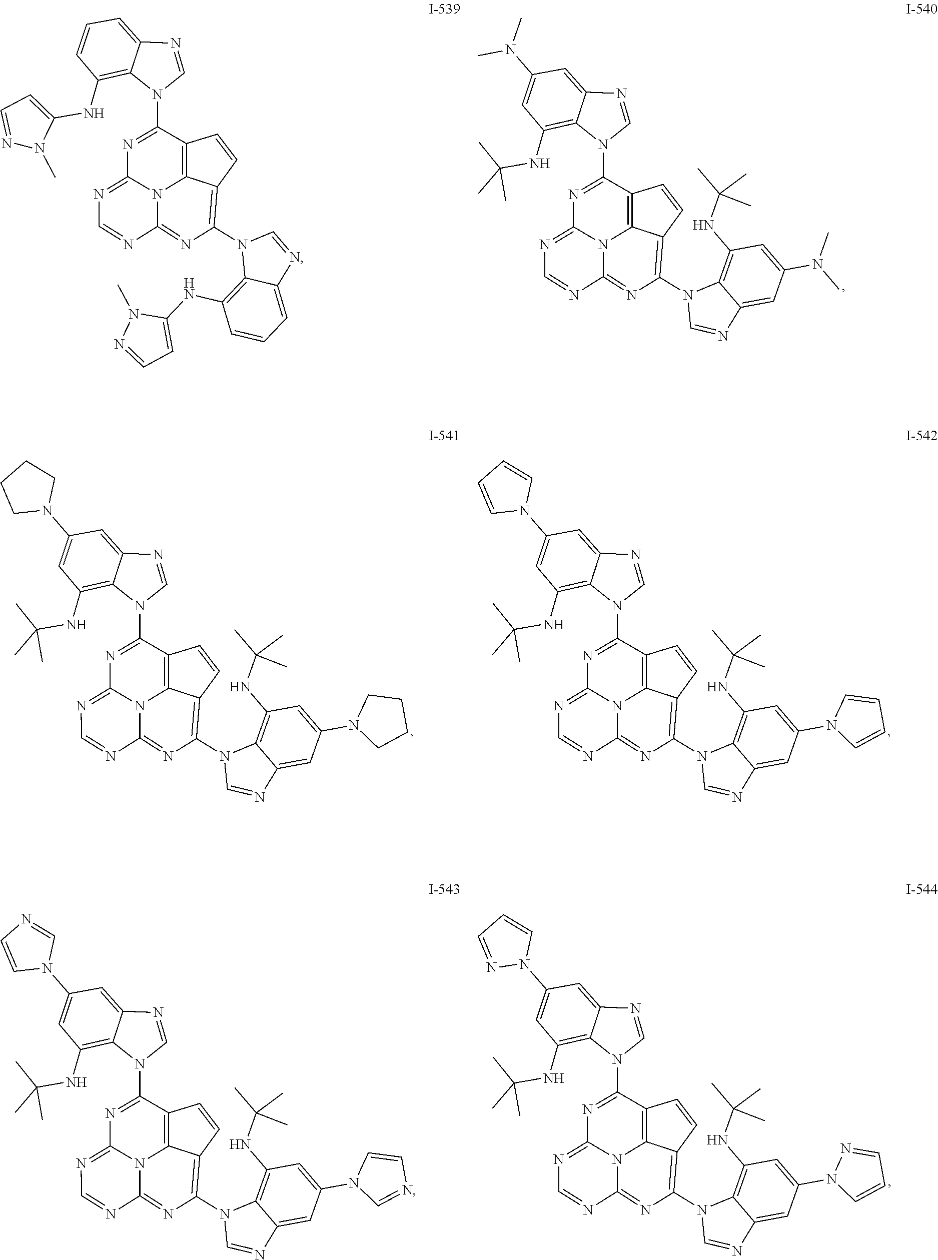

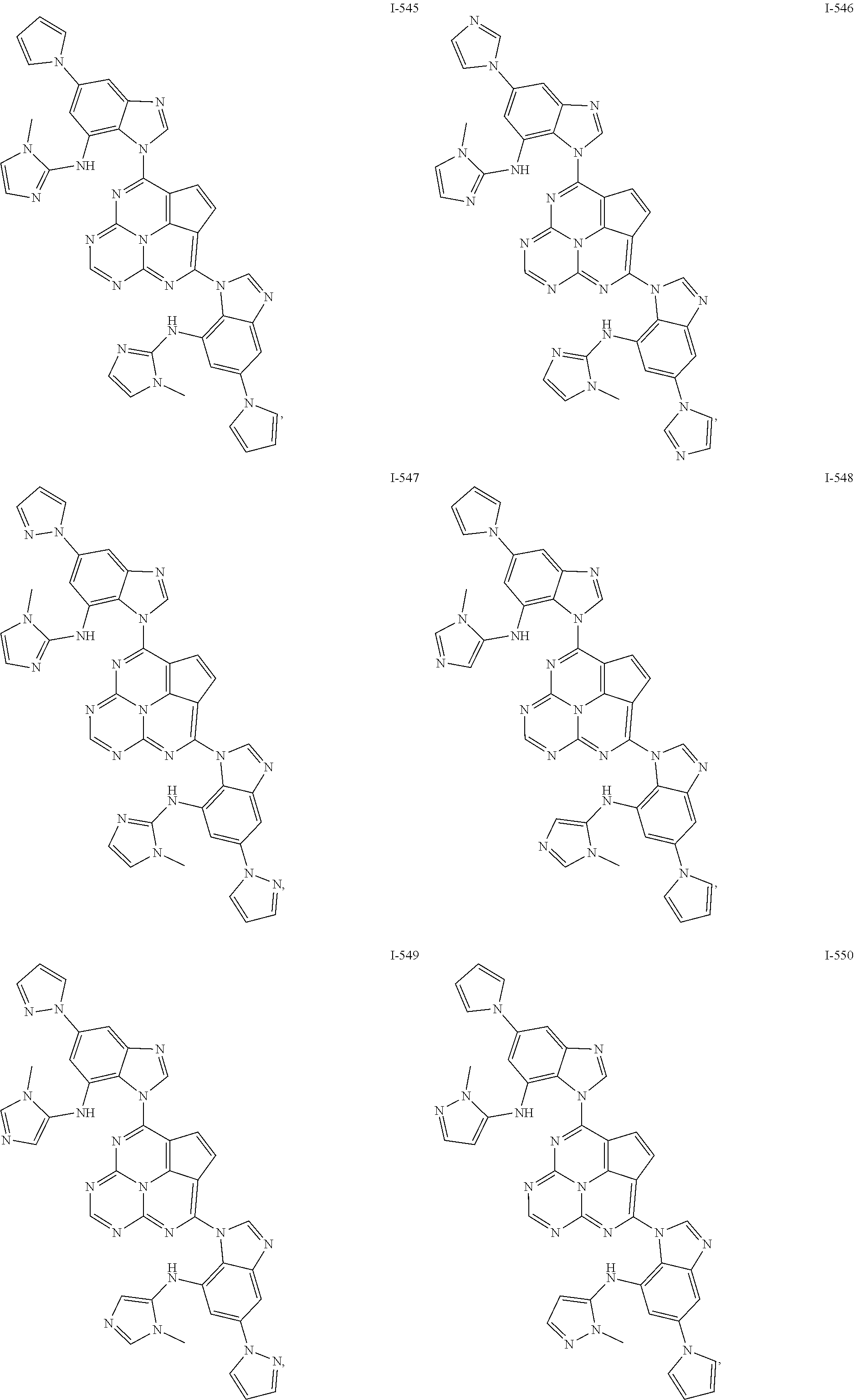

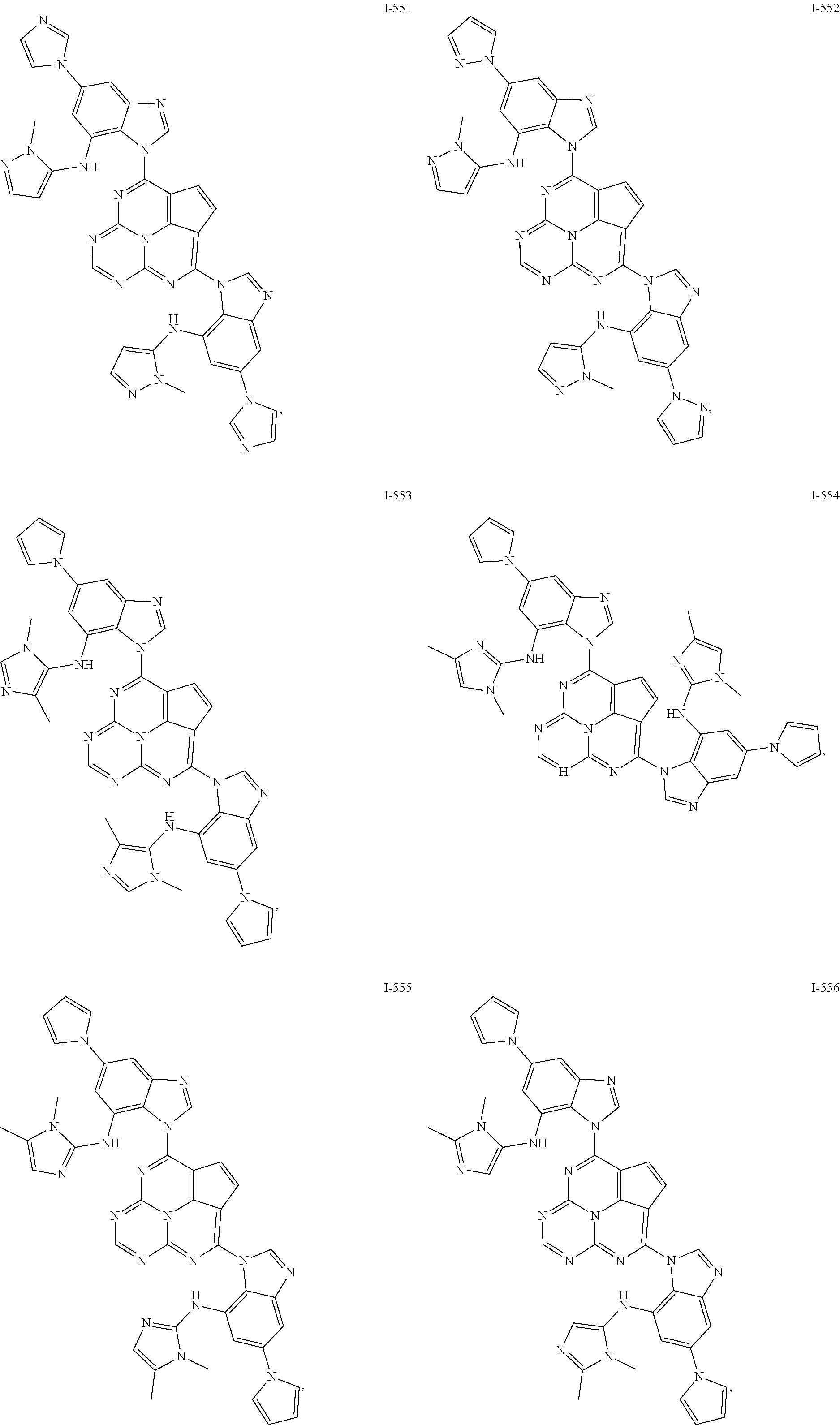

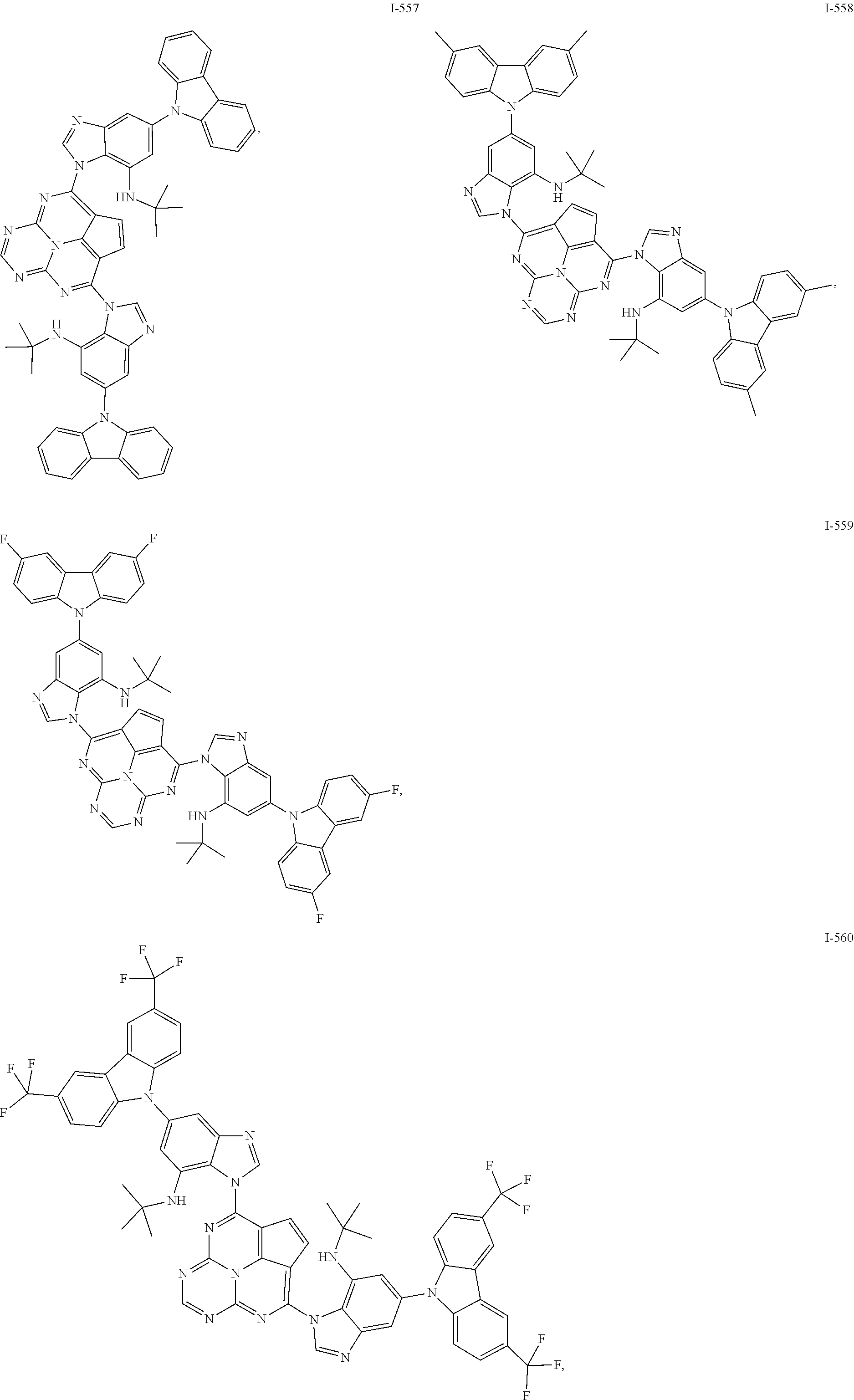

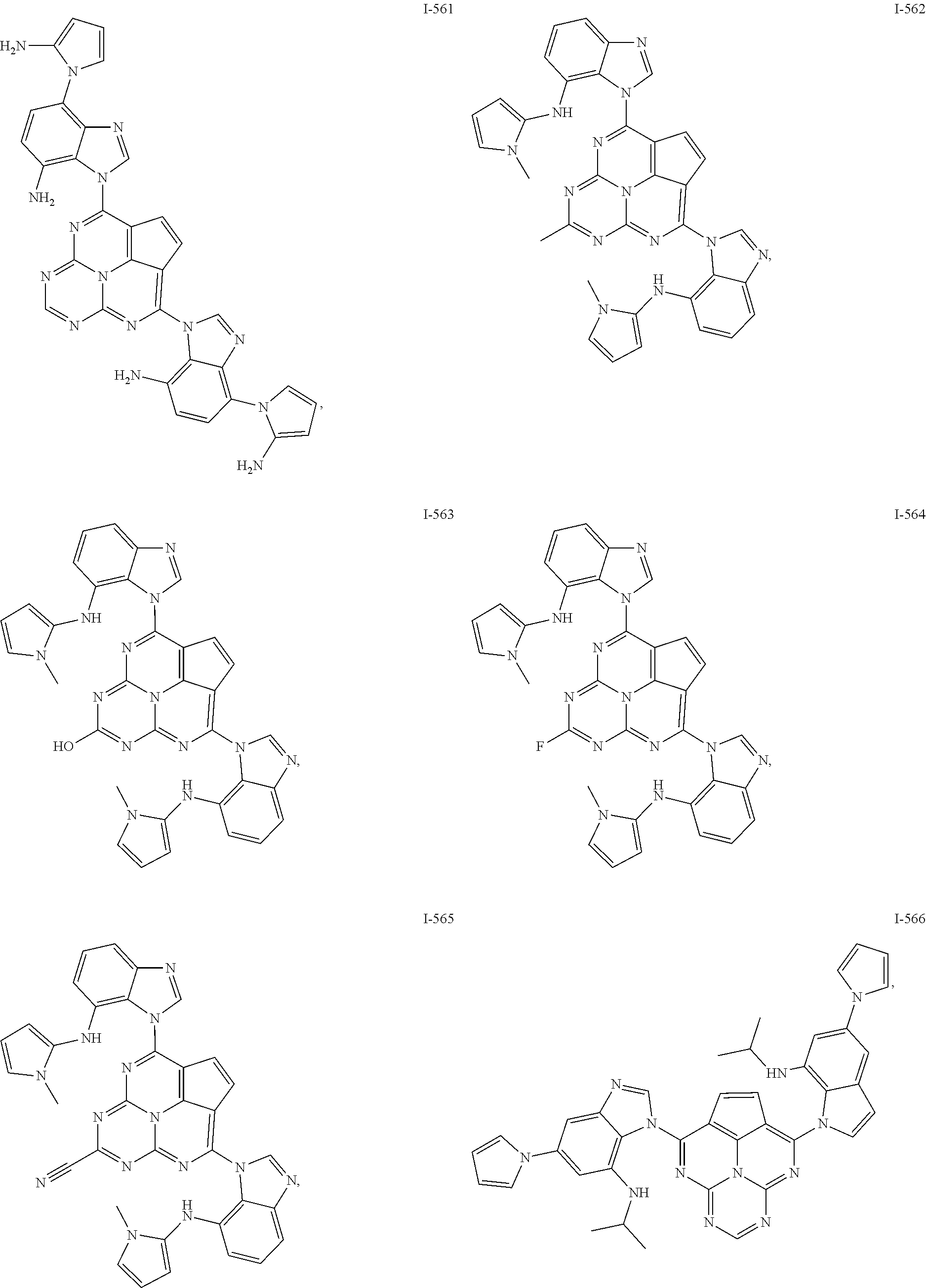

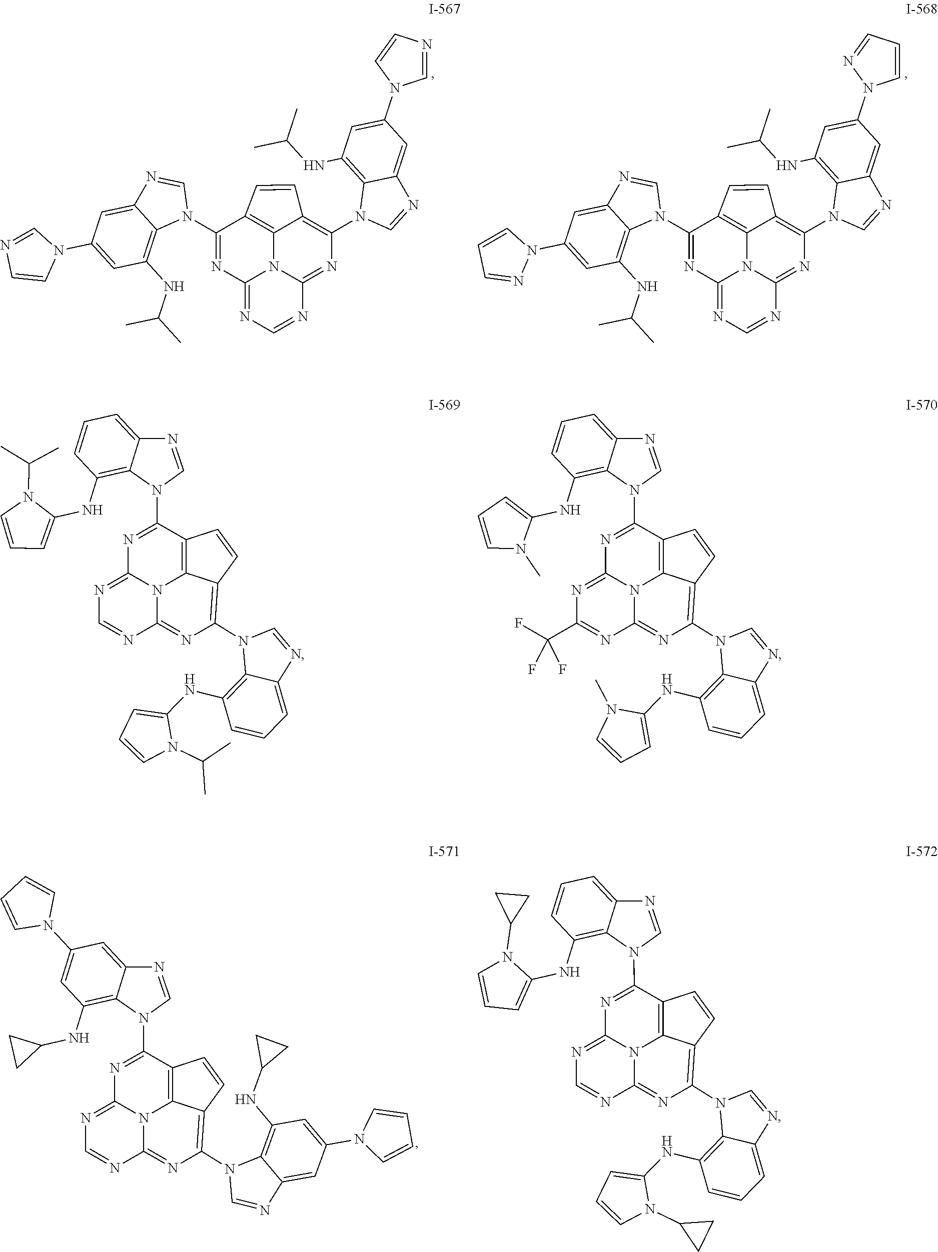

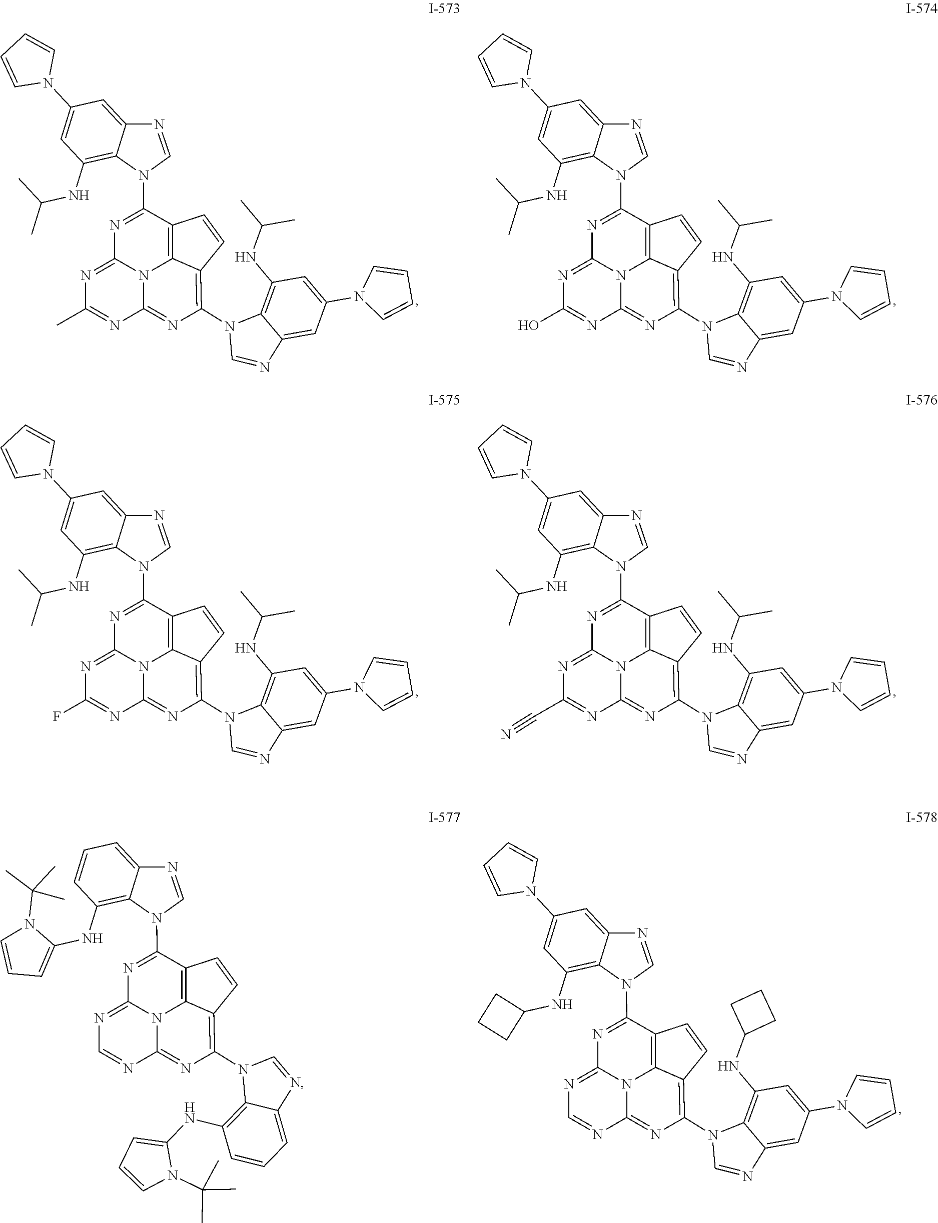

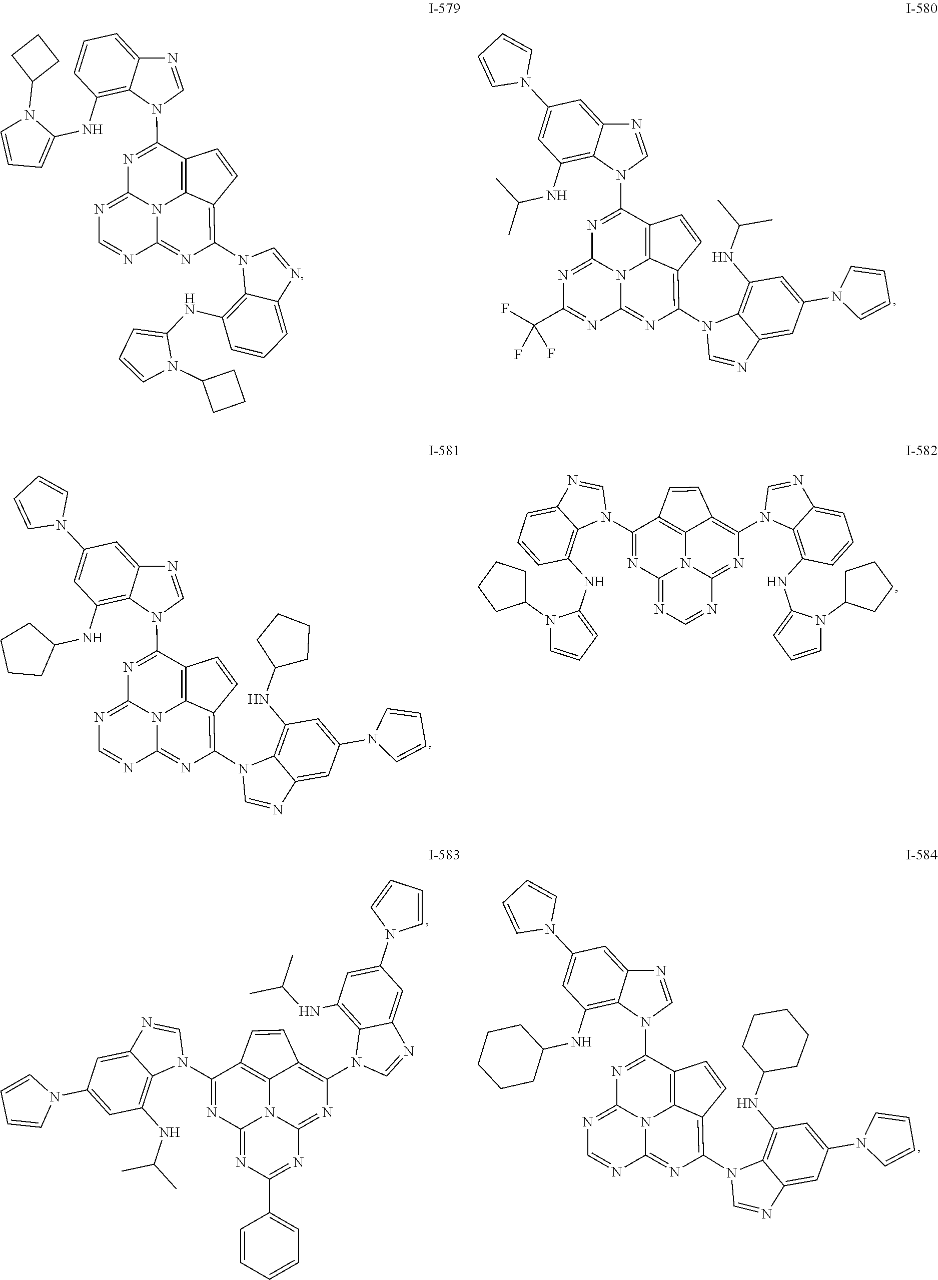

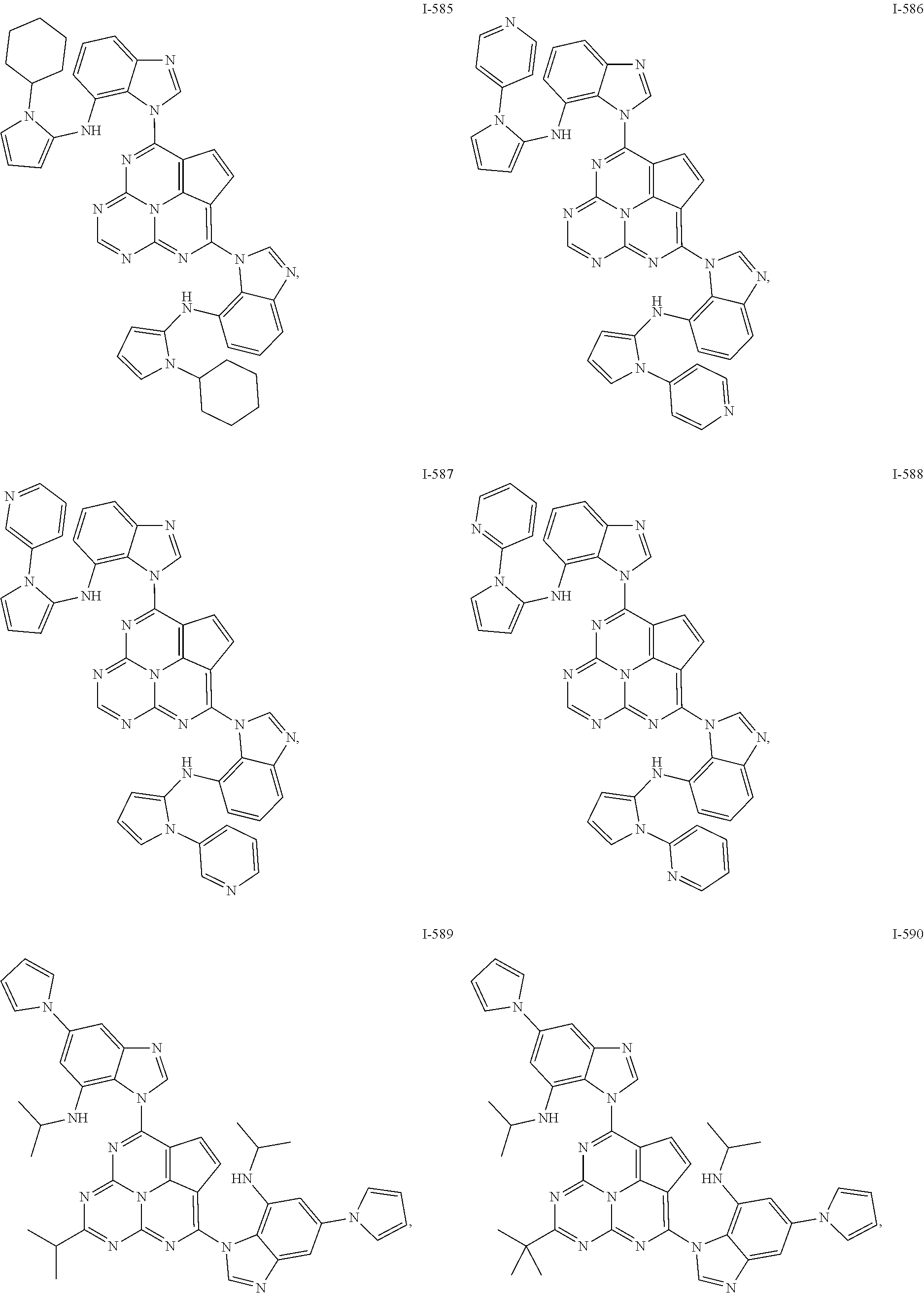

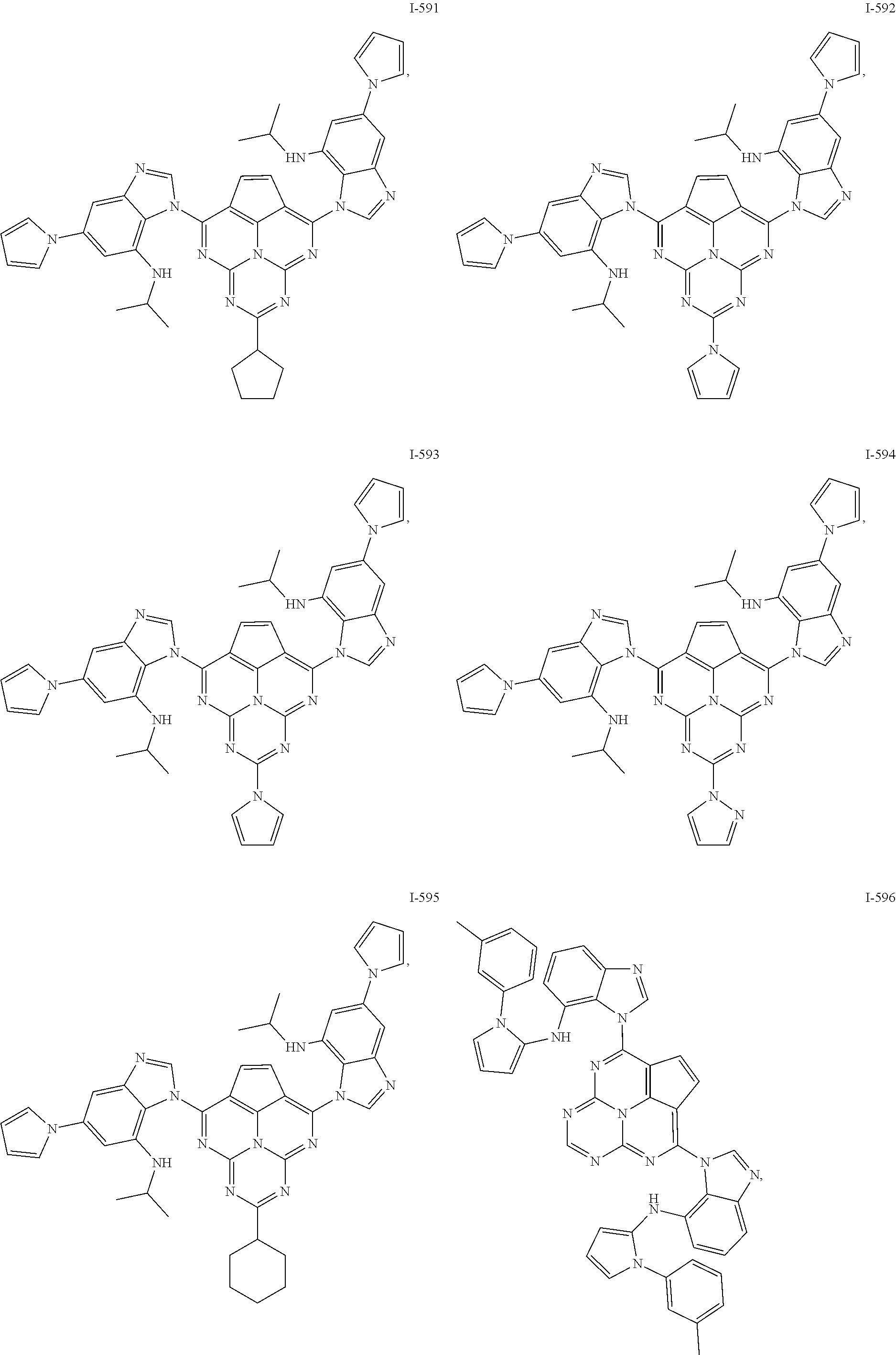

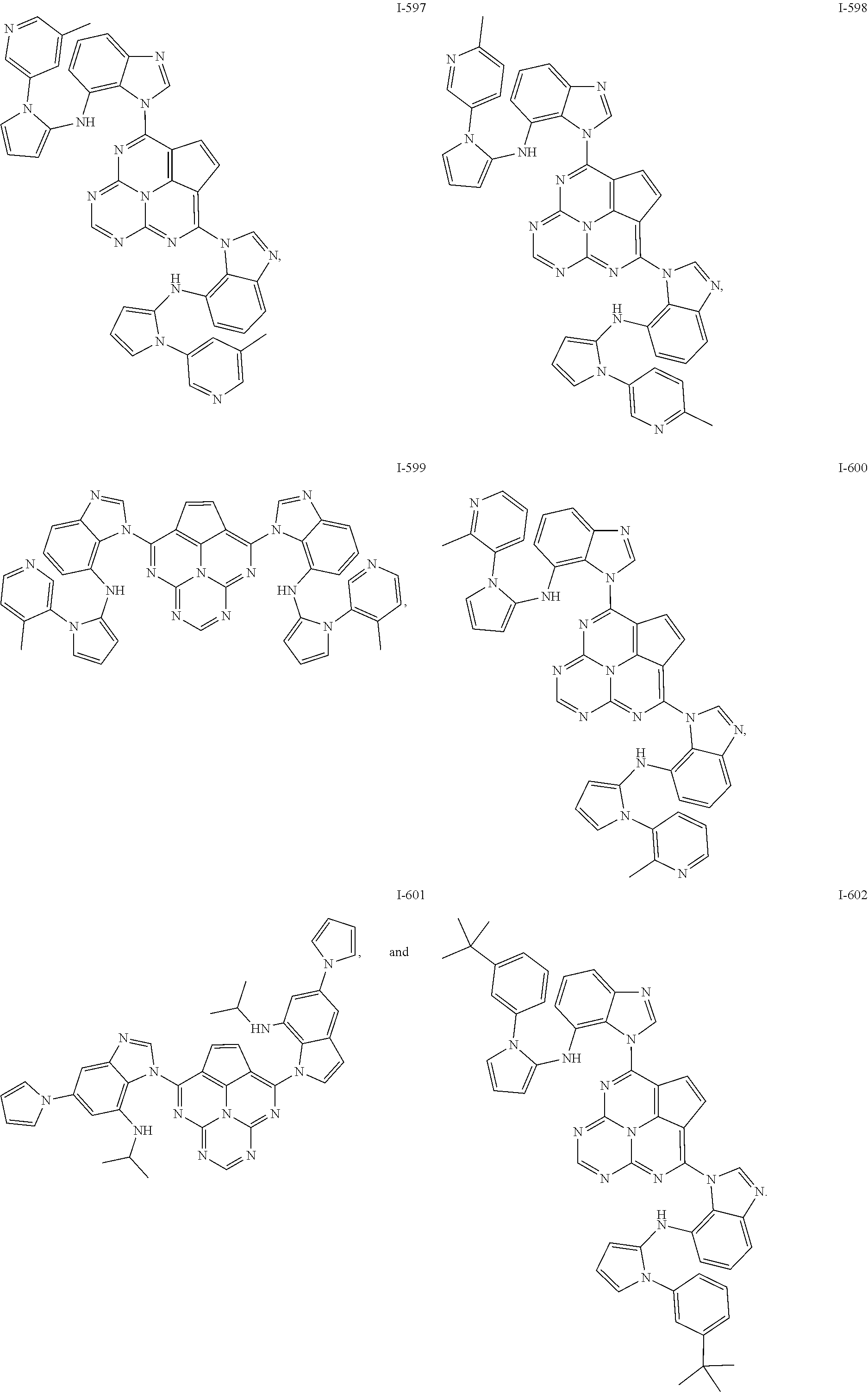

18. The compound of any one of claims 14 to 16, wherein the compound has a structure of Formula I-b 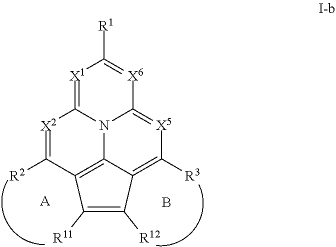
wherein ring A and ring B are each independently a 5-membered or 6-membered carbocycle or heterocycle, optionally an aromatic or heteroaromatic cycle, unsubstituted or substituted with one or more substituents independently selected from R¹⁰.

19. The compound of claim 18, wherein the heterocycle is a nitrogen-containing heterocycle.

20. The compound of claim 18 or 19, wherein R¹¹ and R¹² are nitrogen.

21. The compound of any one of claims 18 to 20, wherein the compound is selected from

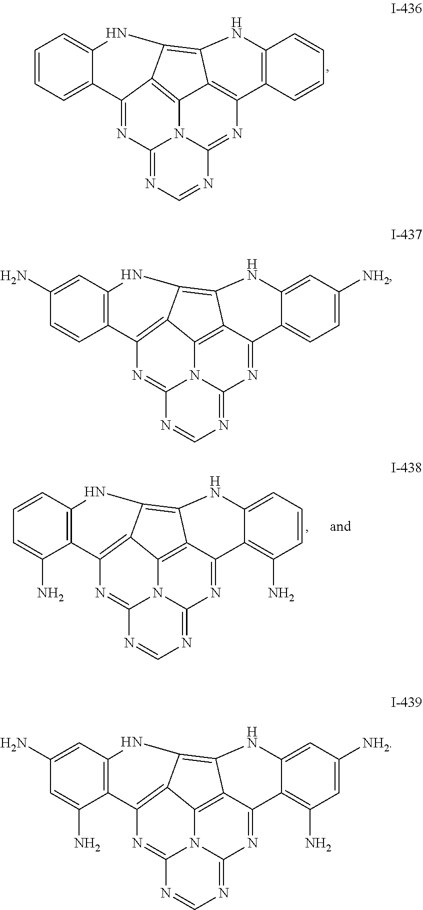 [SMILES: c1ccc2c(c1)[nH]c1c3[nH]c4ccccc4c4nc5ncnc6nc2c1c(c43)n65 ; Nc1ccc2c(c1)[nH]c1c3[nH]c4cc(N)ccc4c4nc5ncnc6nc2c1c(c43)n65 ; Nc1cccc2[nH]c3c4[nH]c5cccc(N)c5c5nc6ncnc7nc(c12)c3c(c54)n76 ; Nc1cc(N)c2c(c1)[nH]c1c3[nH]c4cc(N)cc(N)c4c4nc5ncnc6nc2c1c(c43)n65]

22. The compound of any one of claims 1, 2, and 9 to 12, wherein the compound has a structure of Formula I-c 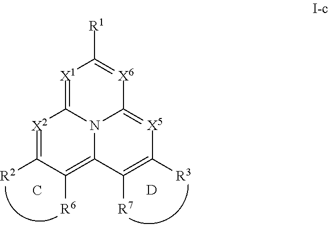
wherein ring C and ring D are each independently a 5-membered or 6-membered carbocycle or heterocycle, optionally an aromatic or heteroaromatic cycle, unsubstituted or substituted with one or more substituents independently selected from R¹⁰.

23. The compound of claim 22, wherein ring C and ring D are each independently selected from nitrogen-containing heterocycles and sulfur-containing heterocycles.

24. The compound of claim 22 or 23, wherein the compound is

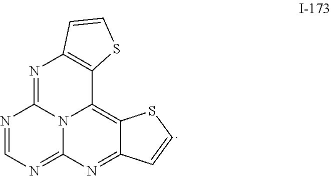 [SMILES: C1=NC2=Nc3ccsc3C3=c4sccc4=NC(=N1)N23]

25. The compound of any one of claims 1 to 12, wherein the compound has a structure of Formula I-d 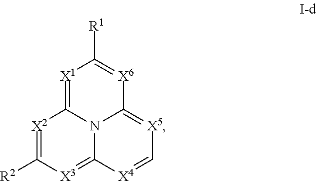
and wherein R¹ and R² are each independently selected from aryl and heteroaryl, each unsubstituted or substituted with one or more substituents independently selected from R¹⁰.

26. The compound of claim 25, wherein R¹ and R² are each independently selected from phenyl, pyrrole, furan, thiophene, indole, benzofuran, benzothiophene, benzoimidazole, indazole, indoline, quinolinone, and pyridine.

27. The compound of claim 25 or 26, wherein the compound is selected from

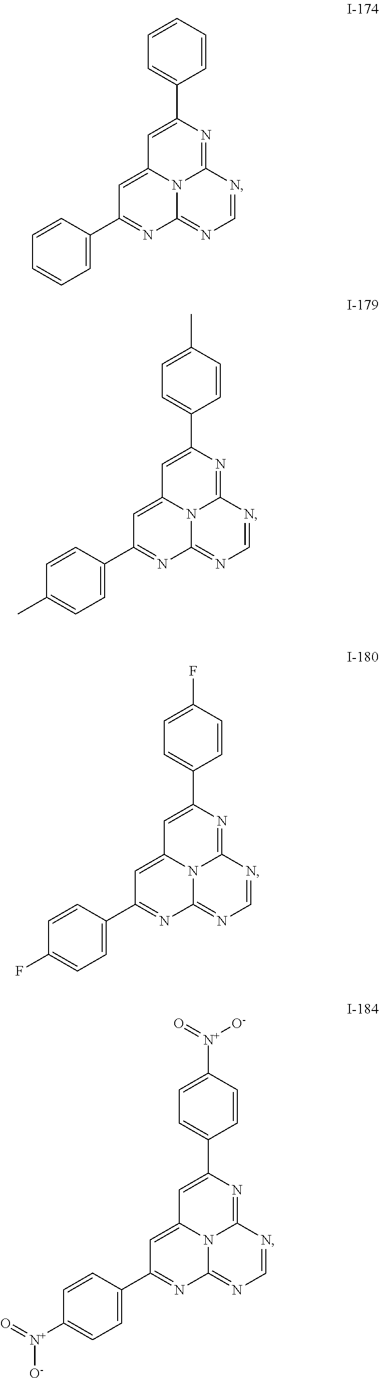 [SMILES: C1=NC2=NC(c3ccccc3)=CC3=CC(c4ccccc4)=NC(=N1)N32 ; Cc1ccc(C2=CC3=CC(c4ccc(C)cc4)=NC4=NC=NC(=N2)N34)cc1 ; Fc1ccc(C2=CC3=CC(c4ccc(F)cc4)=NC4=NC=NC(=N2)N34)cc1 ; O=[N+]([O-])c1ccc(C2=CC3=CC(c4ccc([N+](=O)[O-])cc4)=NC4=NC=NC(=N2)N34)cc1]

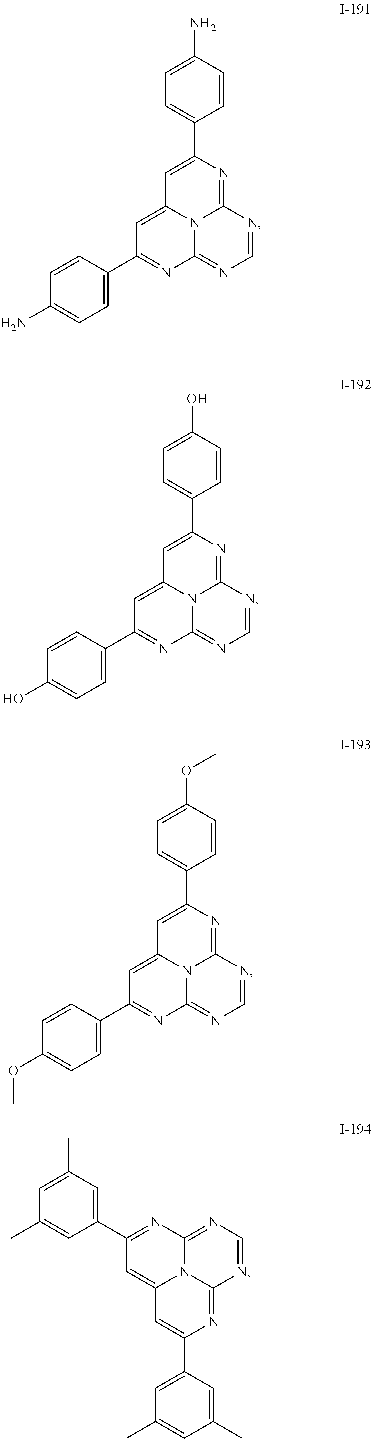

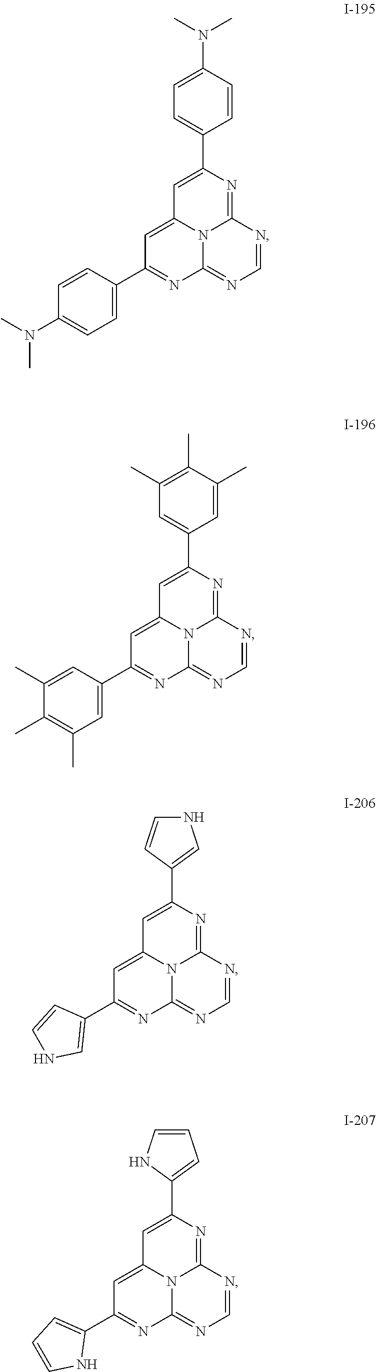

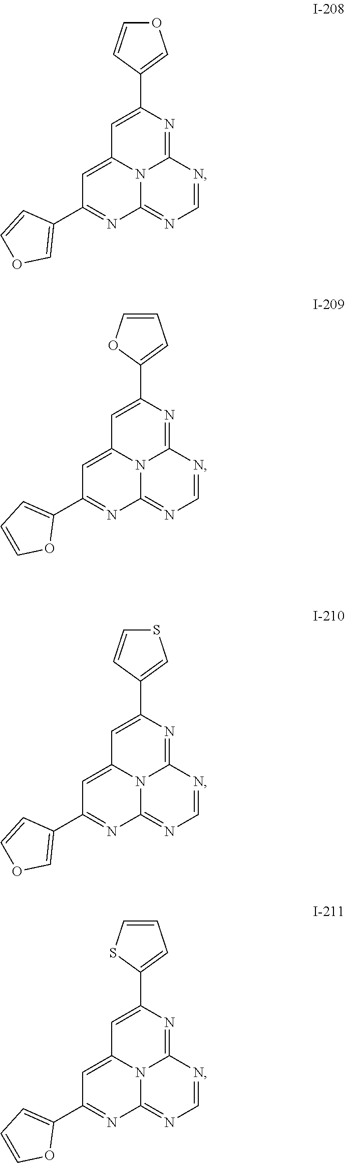 [SMILES: C1=NC2=NC(c3ccoc3)=CC3=CC(c4ccoc4)=NC(=N1)N32 ; C1=NC2=NC(c3ccco3)=CC3=CC(c4ccco4)=NC(=N1)N32 ; C1=NC2=NC(c3ccsc3)=CC3=CC(c4ccoc4)=NC(=N1)N32 ; C1=NC2=NC(c3cccs3)=CC3=CC(c4ccco4)=NC(=N1)N32]

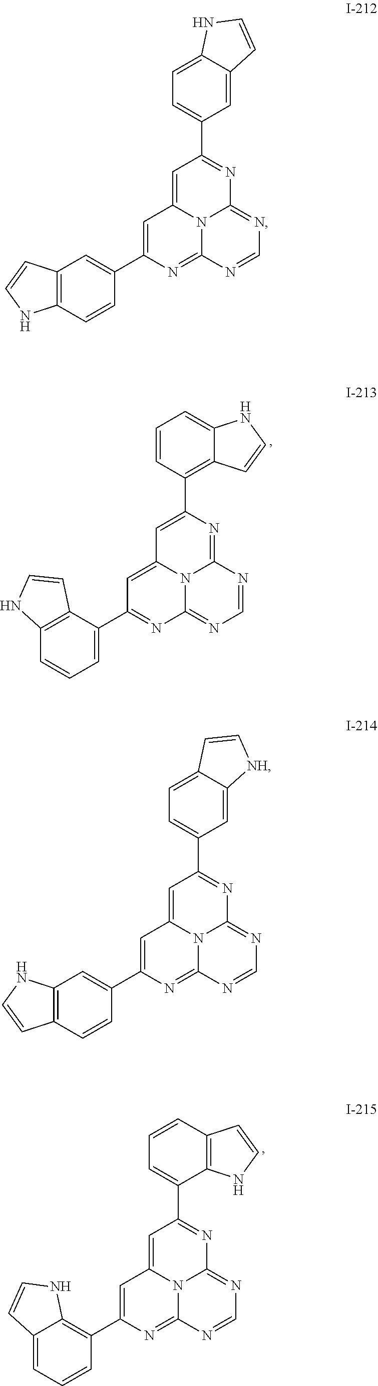 [SMILES: C1=NC2=NC(c3ccc4[nH]ccc4c3)=CC3=CC(c4ccc5[nH]ccc5c4)=NC(=N1)N32 ; C1=NC2=NC(c3cccc4[nH]ccc34)=CC3=CC(c4cccc5[nH]ccc45)=NC(=N1)N32 ; C1=NC2=NC(c3ccc4cc[nH]c4c3)=CC3=CC(c4ccc5cc[nH]c5c4)=NC(=N1)N32 ; C1=NC2=NC(c3cccc4cc[nH]c34)=CC3=CC(c4cccc5cc[nH]c45)=NC(=N1)N32]

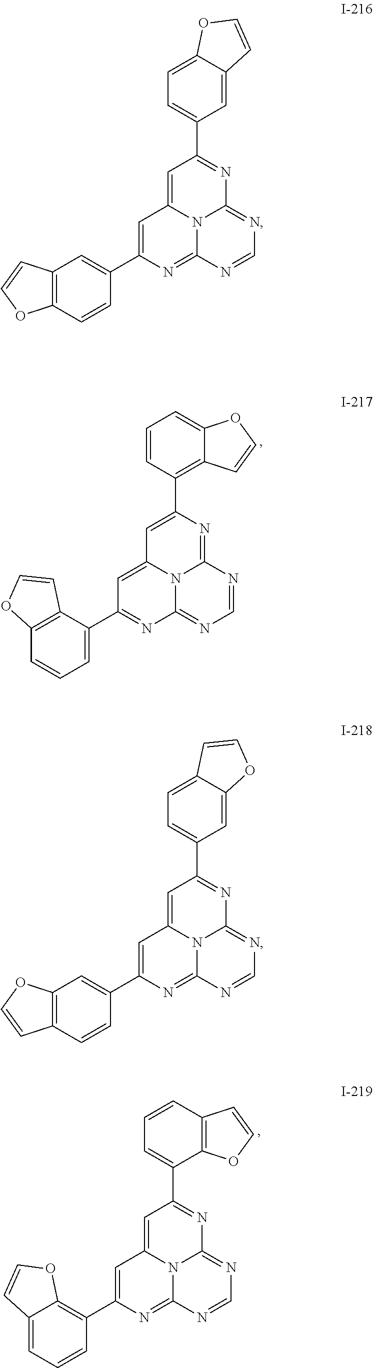 [SMILES: C1=NC2=NC(c3ccc4occc4c3)=CC3=CC(c4ccc5occc5c4)=NC(=N1)N32 ; C1=NC2=NC(c3cccc4occc34)=CC3=CC(c4cccc5occc45)=NC(=N1)N32 ; C1=NC2=NC(c3ccc4ccoc4c3)=CC3=CC(c4ccc5ccoc5c4)=NC(=N1)N32 ; C1=NC2=NC(c3cccc4ccoc34)=CC3=CC(c4cccc5ccoc45)=NC(=N1)N32]

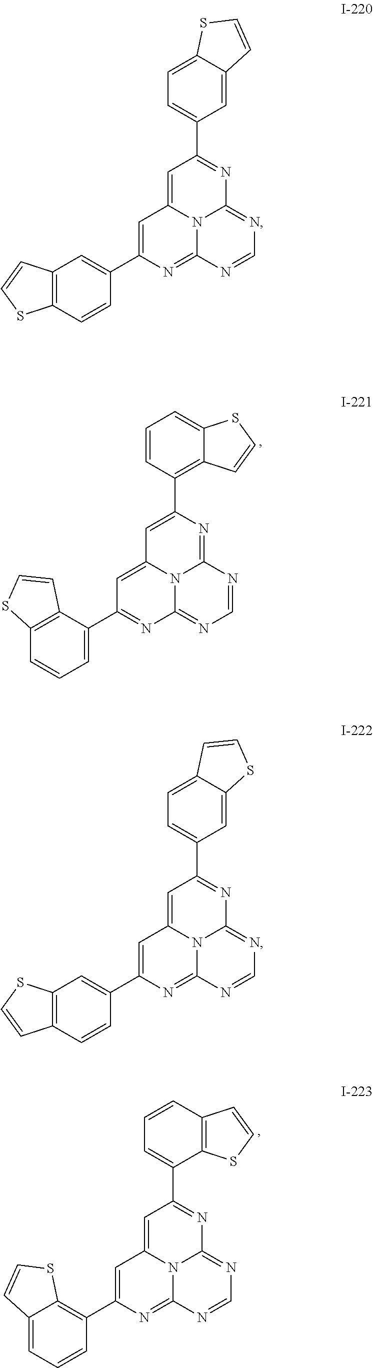 [SMILES: C1=NC2=NC(c3ccc4sccc4c3)=CC3=CC(c4ccc5sccc5c4)=NC(=N1)N32 ; C1=NC2=NC(c3cccc4sccc34)=CC3=CC(c4cccc5sccc45)=NC(=N1)N32 ; C1=NC2=NC(c3ccc4ccsc4c3)=CC3=CC(c4ccc5ccsc5c4)=NC(=N1)N32 ; C1=NC2=NC(c3cccc4ccsc34)=CC3=CC(c4cccc5ccsc45)=NC(=N1)N32]

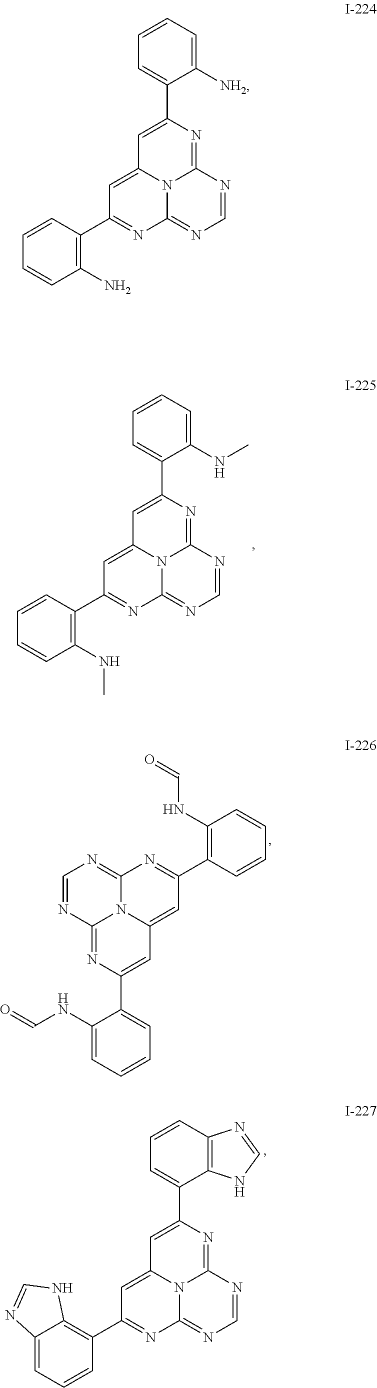 [SMILES: Nc1ccccc1C1=CC2=CC(c3ccccc3N)=NC3=NC=NC(=N1)N23 ; CNc1ccccc1C1=CC2=CC(c3ccccc3NC)=NC3=NC=NC(=N1)N23 ; O=CNc1ccccc1C1=CC2=CC(c3ccccc3NC=O)=NC3=NC=NC(=N1)N23 ; C1=NC2=NC(c3cccc4nc[nH]c34)=CC3=CC(c4cccc5nc[nH]c45)=NC(=N1)N32]

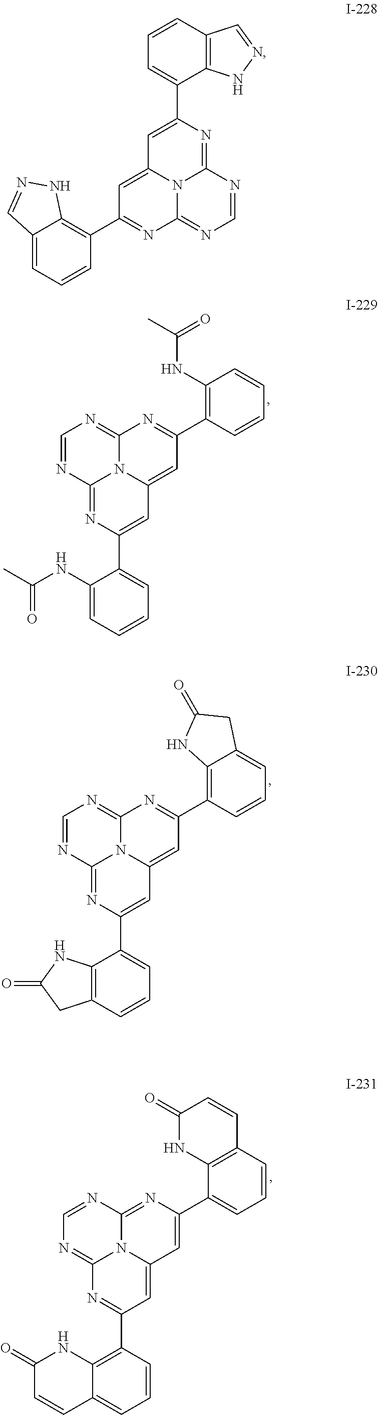 [SMILES: C1=NC2=NC(c3cccc4cn[nH]c34)=CC3=CC(c4cccc5cn[nH]c45)=NC(=N1)N32 ; CC(=O)Nc1ccccc1C1=CC2=CC(c3ccccc3NC(C)=O)=NC3=NC=NC(=N1)N23 ; O=C1Cc2cccc(C3=CC4=CC(c5cccc6c5NC(=O)C6)=NC5=NC=NC(=N3)N45)c2N1 ; O=c1ccc2cccc(C3=CC4=CC(c5cccc6ccc(=O)[nH]c56)=NC5=NC=NC(=N3)N45)c2[nH]1]

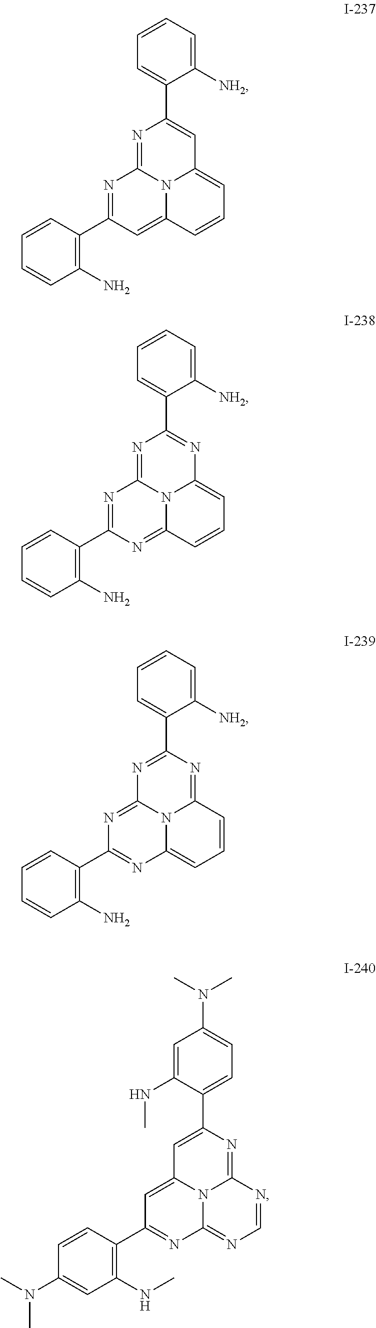 [SMILES: Nc1ccccc1C1=CC2=CC=CC3=CC(c4ccccc4N)=NC(=N1)N23 ; Nc1ccccc1C1=NC2=CC=CC3=NC(c4ccccc4N)=NC(=N1)N23 ; Nc1ccccc1C1=NC2=CC=CC3=NC(c4ccccc4N)=NC(=N1)N23 ; CNc1cc(N(C)C)ccc1C1=CC2=CC(c3ccc(N(C)C)cc3NC)=NC3=NC=NC(=N1)N23]

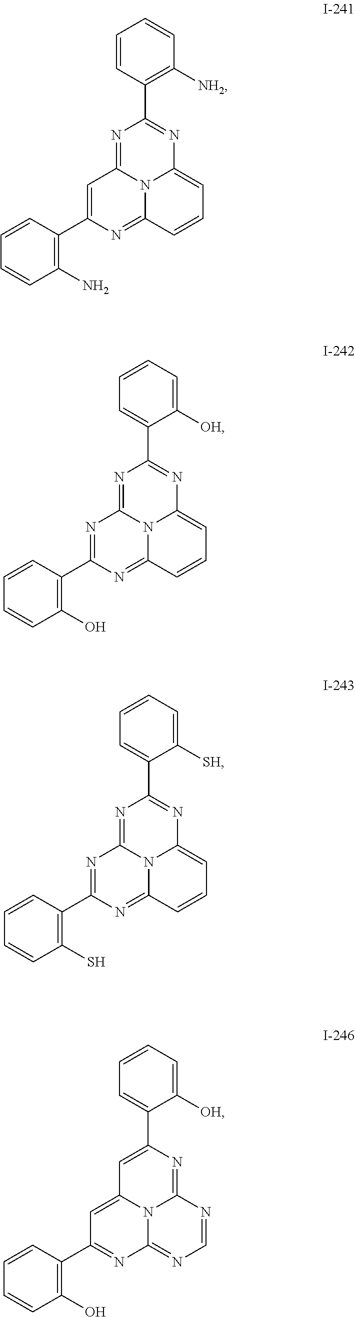 [SMILES: Nc1ccccc1C1=CC2=NC(c3ccccc3N)=NC3=CC=CC(=N1)N32 ; Oc1ccccc1C1=NC2=CC=CC3=NC(c4ccccc4O)=NC(=N1)N23 ; Sc1ccccc1C1=NC2=CC=CC3=NC(c4ccccc4S)=NC(=N1)N23 ; Oc1ccccc1C1=CC2=CC(c3ccccc3O)=NC3=NC=NC(=N1)N23]

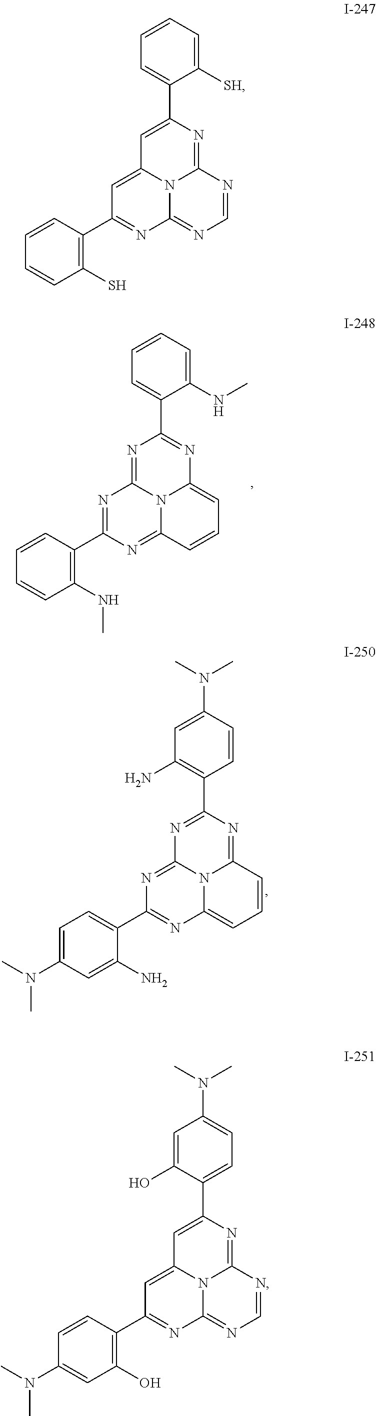

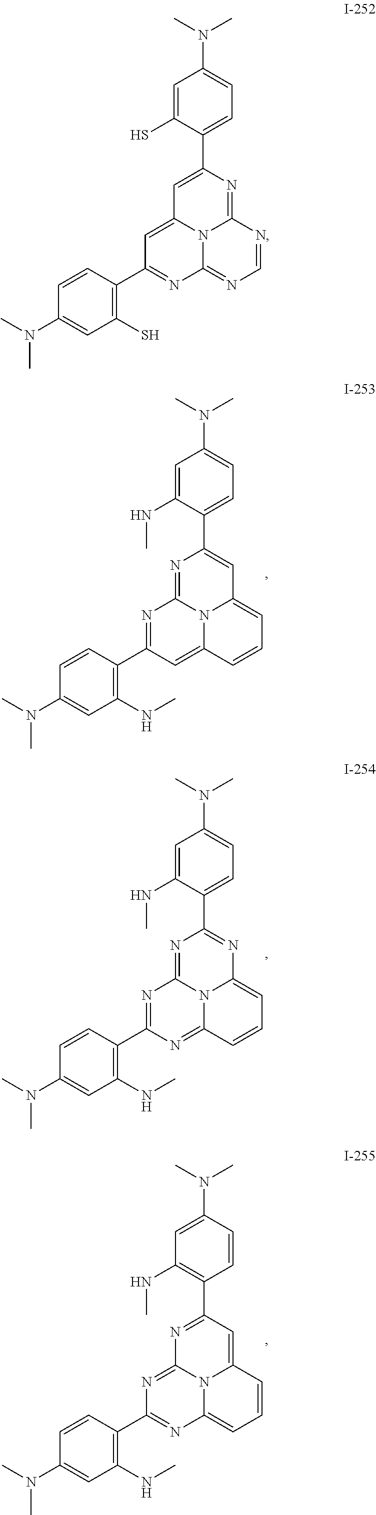 [SMILES: CN(C)c1ccc(C2=CC3=CC(c4ccc(N(C)C)cc4S)=NC4=NC=NC(=N2)N34)c(S)c1 ; CNc1cc(N(C)C)ccc1C1=CC2=CC=CC3=CC(c4ccc(N(C)C)cc4NC)=NC(=N1)N23 ; CNc1cc(N(C)C)ccc1C1=NC2=CC=CC3=NC(c4ccc(N(C)C)cc4NC)=NC(=N1)N23 ; CNc1cc(N(C)C)ccc1C1=NC2=NC(c3ccc(N(C)C)cc3NC)=NC3=CC=CC(=C1)N32]

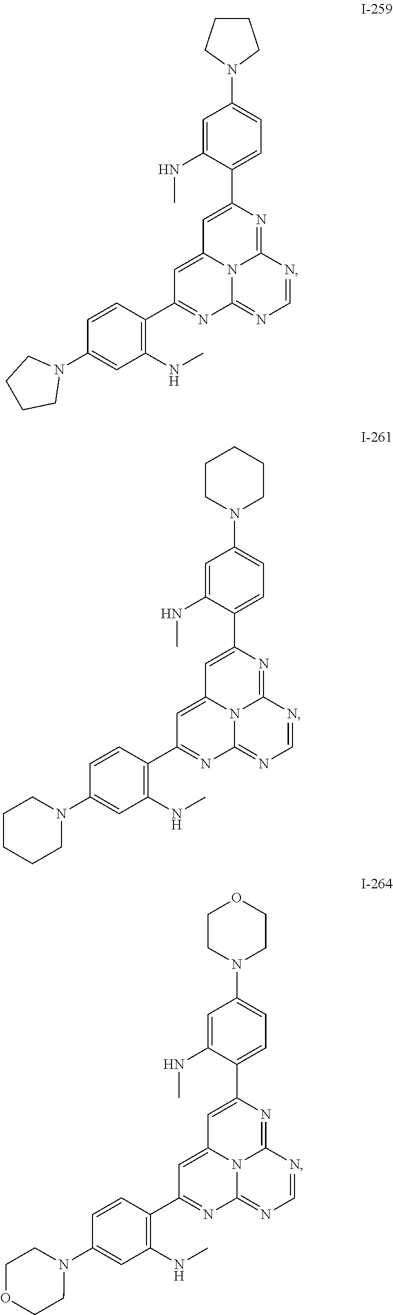 [SMILES: CNc1cc(N2CCCC2)ccc1C1=CC2=CC(c3ccc(N4CCCC4)cc3NC)=NC3=NC=NC(=N1)N23 ; CNc1cc(N2CCCCC2)ccc1C1=CC2=CC(c3ccc(N4CCCCC4)cc3NC)=NC3=NC=NC(=N1)N23 ; CNc1cc(N2CCOCC2)ccc1C1=CC2=CC(c3ccc(N4CCOCC4)cc3NC)=NC3=NC=NC(=N1)N23]

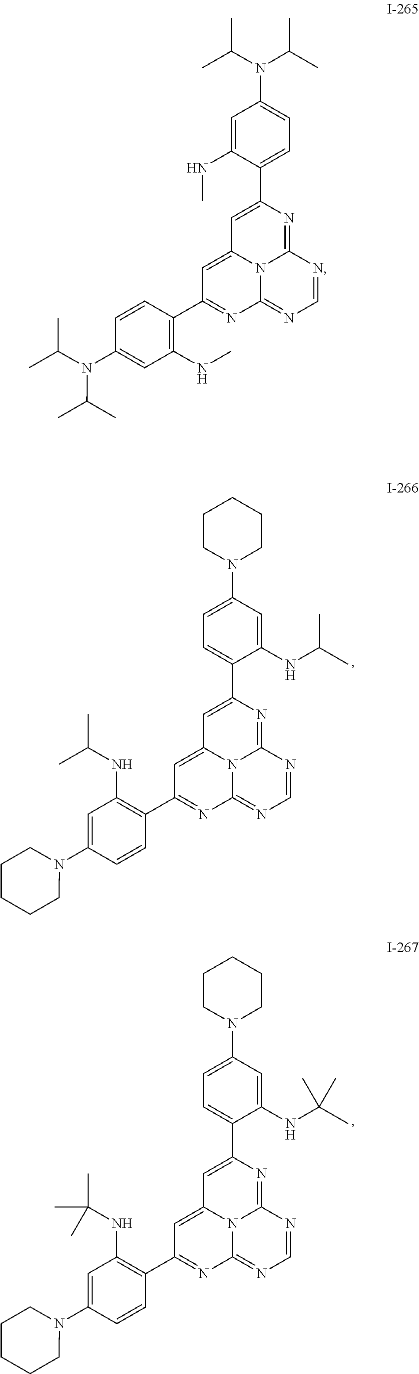 [SMILES: CNc1cc(N(C(C)C)C(C)C)ccc1C1=CC2=CC(c3ccc(N(C(C)C)C(C)C)cc3NC)=NC3=NC=NC(=N1)N23 ; CC(C)Nc1cc(N2CCCCC2)ccc1C1=CC2=CC(c3ccc(N4CCCCC4)cc3NC(C)C)=NC3=NC=NC(=N1)N23 ; CC(C)(C)Nc1cc(N2CCCCC2)ccc1C1=CC2=CC(c3ccc(N4CCCCC4)cc3NC(C)(C)C)=NC3=NC=NC(=N1)N23]

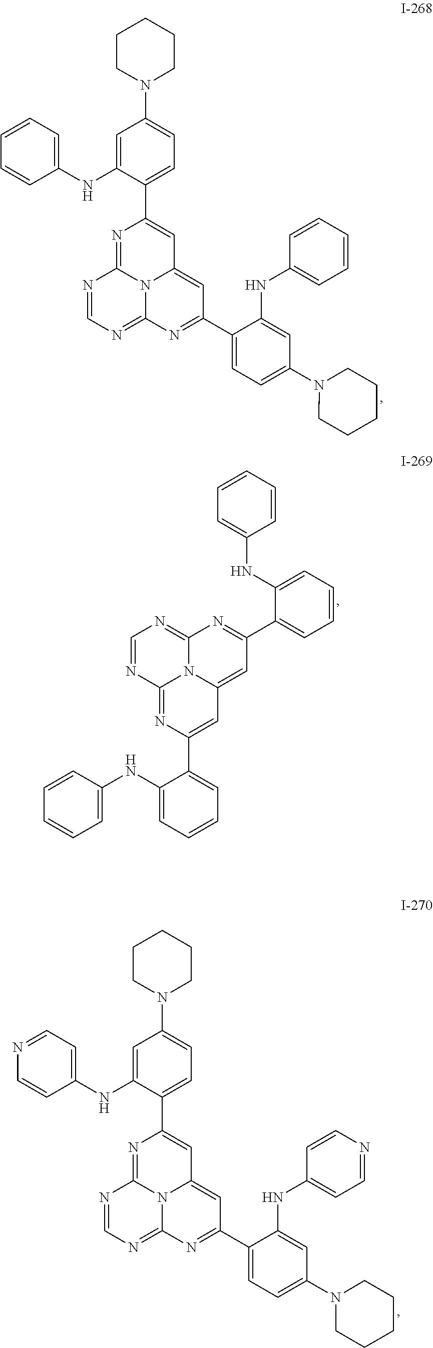 [SMILES: C1=NC2=NC(c3ccc(N4CCCCC4)cc3Nc3ccccc3)=CC3=CC(c4ccc(N5CCCCC5)cc4Nc4ccccc4)=NC(=N1)N32 ; C1=NC2=NC(c3ccccc3Nc3ccccc3)=CC3=CC(c4ccccc4Nc4ccccc4)=NC(=N1)N32 ; C1=NC2=NC(c3ccc(N4CCCCC4)cc3Nc3ccncc3)=CC3=CC(c4ccc(N5CCCCC5)cc4Nc4ccncc4)=NC(=N1)N32]

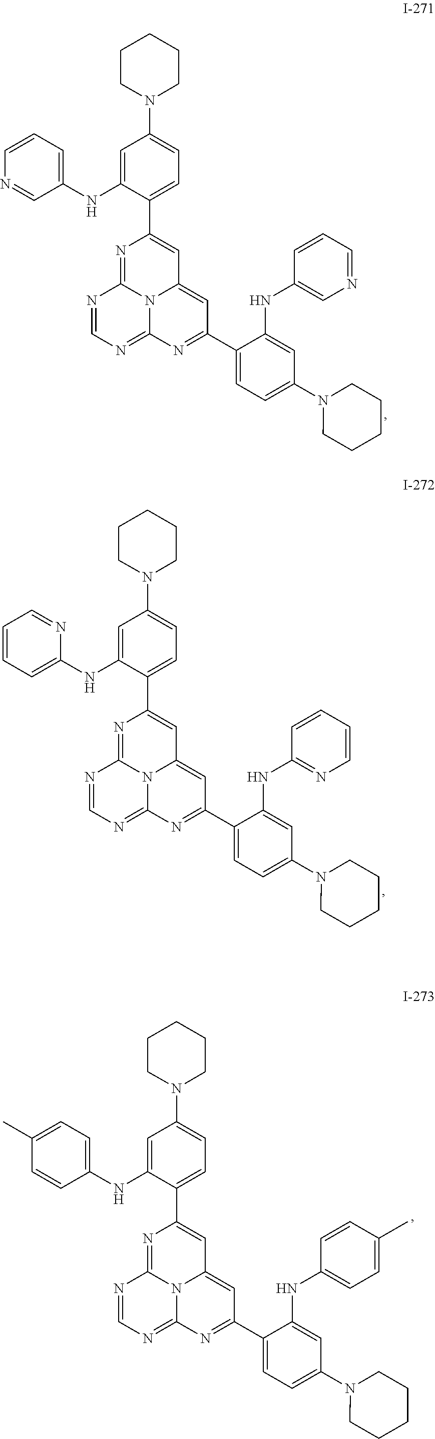

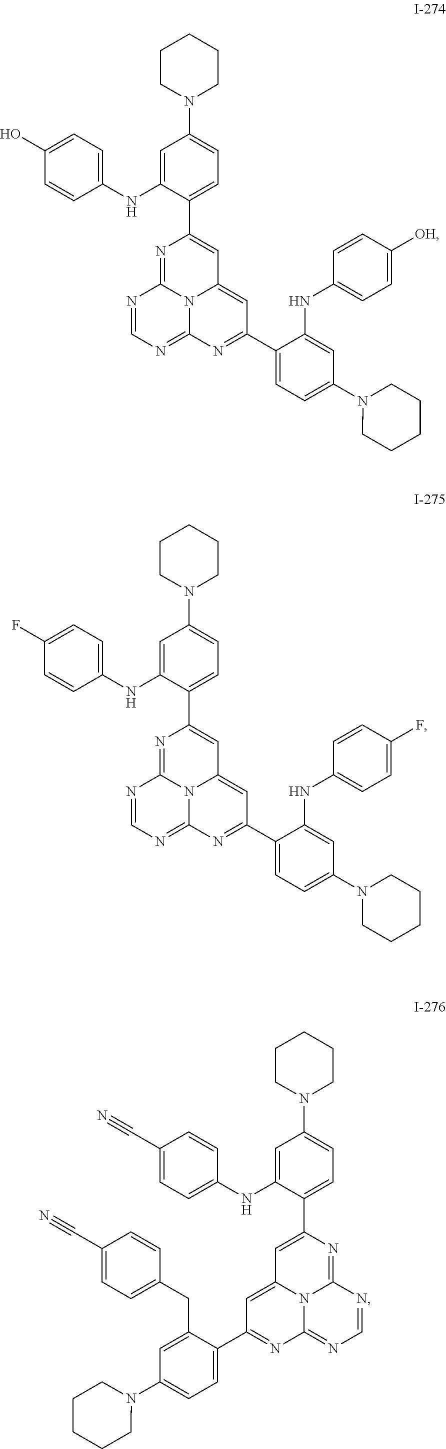 [SMILES: Oc1ccc(Nc2cc(N3CCCCC3)ccc2C2=CC3=CC(c4ccc(N5CCCCC5)cc4Nc4ccc(O)cc4)=NC4=NC=NC(=N2)N34)cc1 ; Fc1ccc(Nc2cc(N3CCCCC3)ccc2C2=CC3=CC(c4ccc(N5CCCCC5)cc4Nc4ccc(F)cc4)=NC4=NC=NC(=N2)N34)cc1 ; N#Cc1ccc(Cc2cc(N3CCCCC3)ccc2C2=NC3=NC=NC4=NC(c5ccc(N6CCCCC6)cc5Nc5ccc(C#N)cc5)=CC(=C2)N34)cc1]

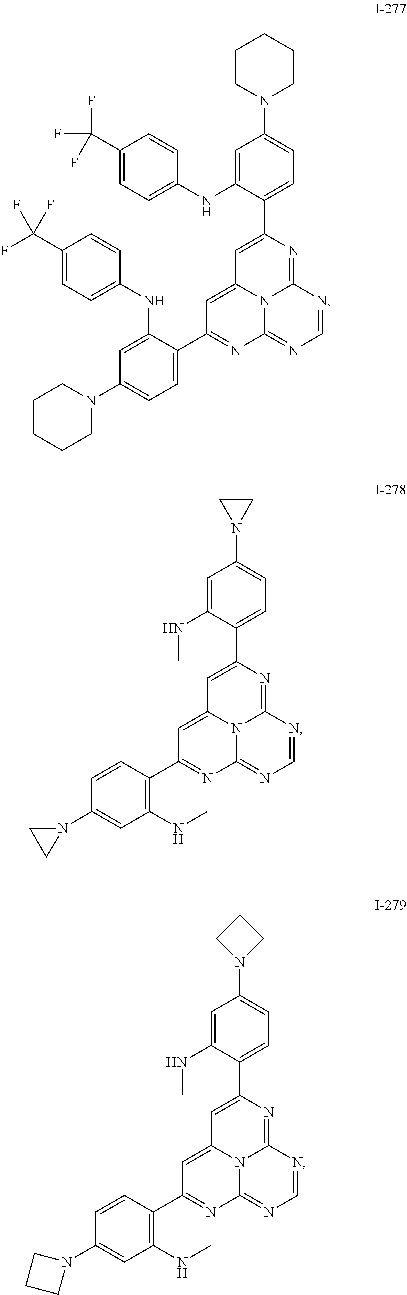 [SMILES: FC(F)(F)c1ccc(Nc2cc(N3CCCCC3)ccc2C2=CC3=CC(c4ccc(N5CCCCC5)cc4Nc4ccc(C(F)(F)F)cc4)=NC4=NC=NC(=N2)N34)cc1 ; CNc1cc(N2CC2)ccc1C1=CC2=CC(c3ccc(N4CC4)cc3NC)=NC3=NC=NC(=N1)N23 ; CNc1cc(N2CCC2)ccc1C1=CC2=CC(c3ccc(N4CCC4)cc3NC)=NC3=NC=NC(=N1)N23]

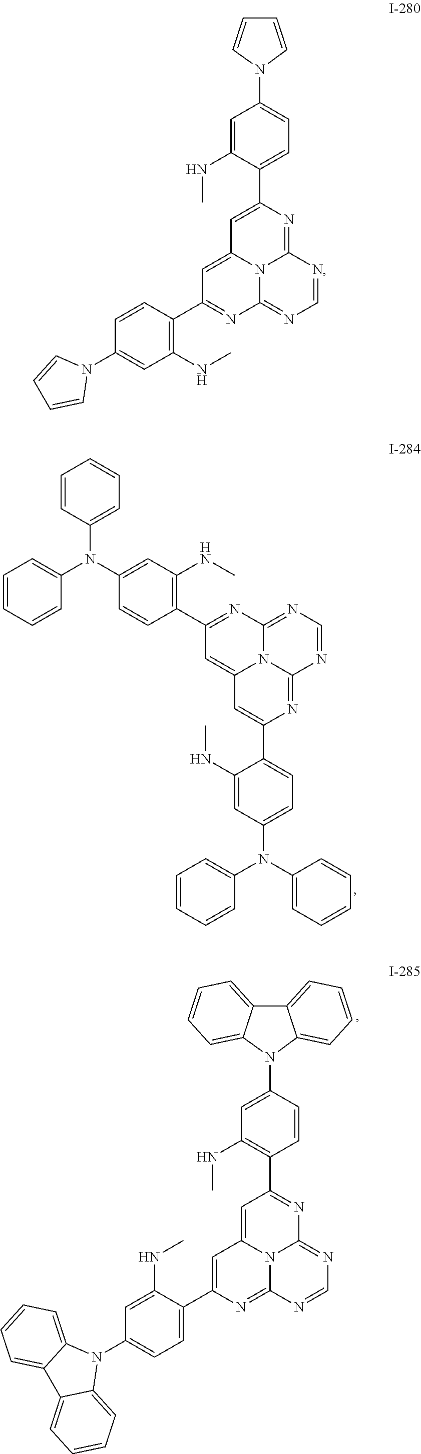 [SMILES: CNc1cc(-n2cccc2)ccc1C1=CC2=CC(c3ccc(-n4cccc4)cc3NC)=NC3=NC=NC(=N1)N23 ; CNc1cc(N(c2ccccc2)c2ccccc2)ccc1C1=CC2=CC(c3ccc(N(c4ccccc4)c4ccccc4)cc3NC)=NC3=NC=NC(=N1)N23 ; CNc1cc(-n2c3ccccc3c3ccccc32)ccc1C1=CC2=CC(c3ccc(-n4c5ccccc5c5ccccc54)cc3NC)=NC3=NC=NC(=N1)N23]

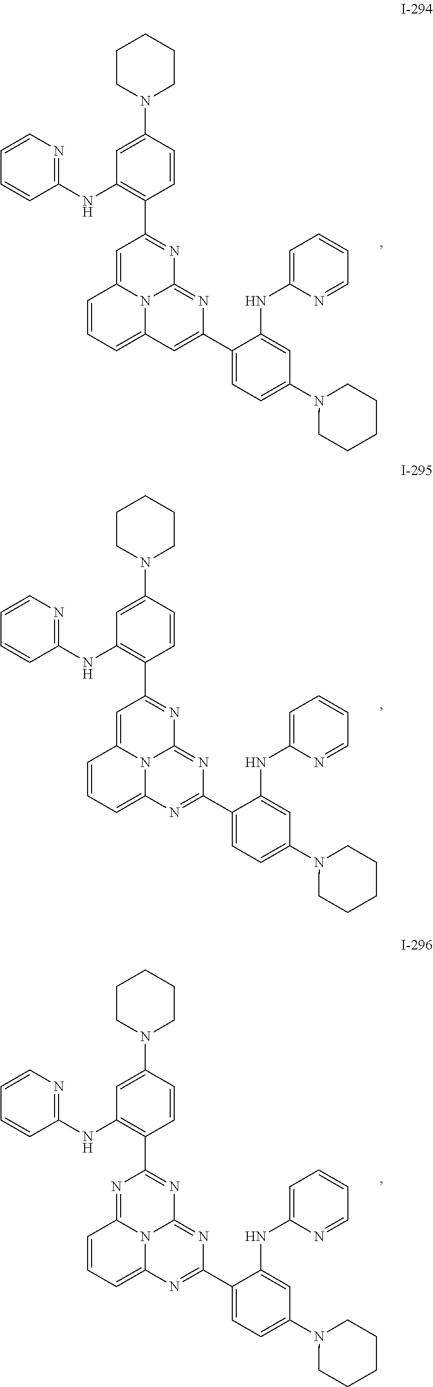 [SMILES: C1=CC2=CC(c3ccc(N4CCCCC4)cc3Nc3ccccn3)=NC3=NC(c4ccc(N5CCCCC5)cc4Nc4ccccn4)=CC(=C1)N23 ; C1=CC2=CC(c3ccc(N4CCCCC4)cc3Nc3ccccn3)=NC3=NC(c4ccc(N5CCCCC5)cc4Nc4ccccn4)=NC(=C1)N23 ; C1=CC2=NC(c3ccc(N4CCCCC4)cc3Nc3ccccn3)=NC3=NC(c4ccc(N5CCCCC5)cc4Nc4ccccn4)=NC(=C1)N23]

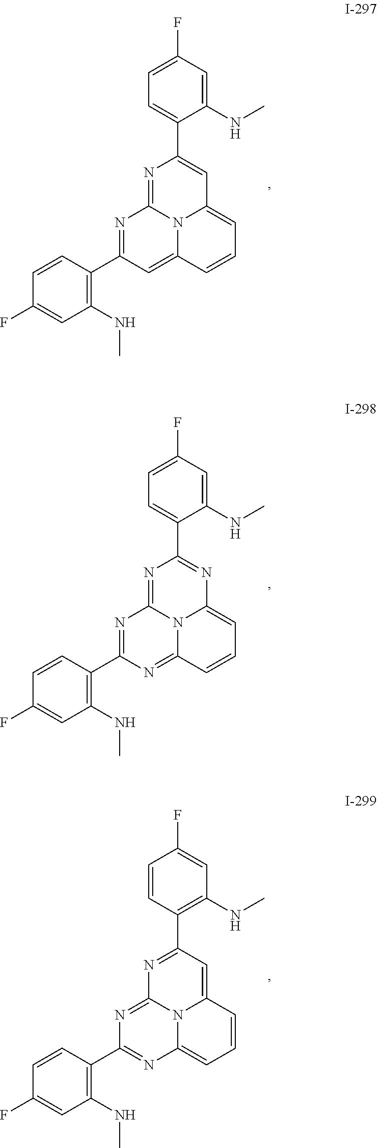 [SMILES: CNc1cc(F)ccc1C1=CC2=CC=CC3=CC(c4ccc(F)cc4NC)=NC(=N1)N23 ; CNc1cc(F)ccc1C1=NC2=CC=CC3=NC(c4ccc(F)cc4NC)=NC(=N1)N23 ; CNc1cc(F)ccc1C1=NC2=NC(c3ccc(F)cc3NC)=NC3=CC=CC(=C1)N32]

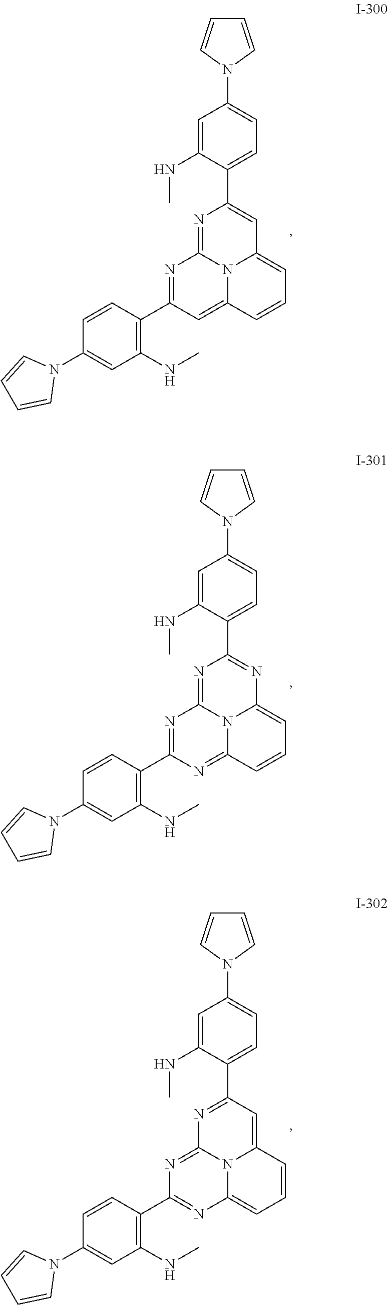 [SMILES: CNc1cc(-n2cccc2)ccc1C1=CC2=CC=CC3=CC(c4ccc(-n5cccc5)cc4NC)=NC(=N1)N23 ; CNc1cc(-n2cccc2)ccc1C1=NC2=CC=CC3=NC(c4ccc(-n5cccc5)cc4NC)=NC(=N1)N23 ; CNc1cc(-n2cccc2)ccc1C1=NC2=NC(c3ccc(-n4cccc4)cc3NC)=NC3=CC=CC(=C1)N32]

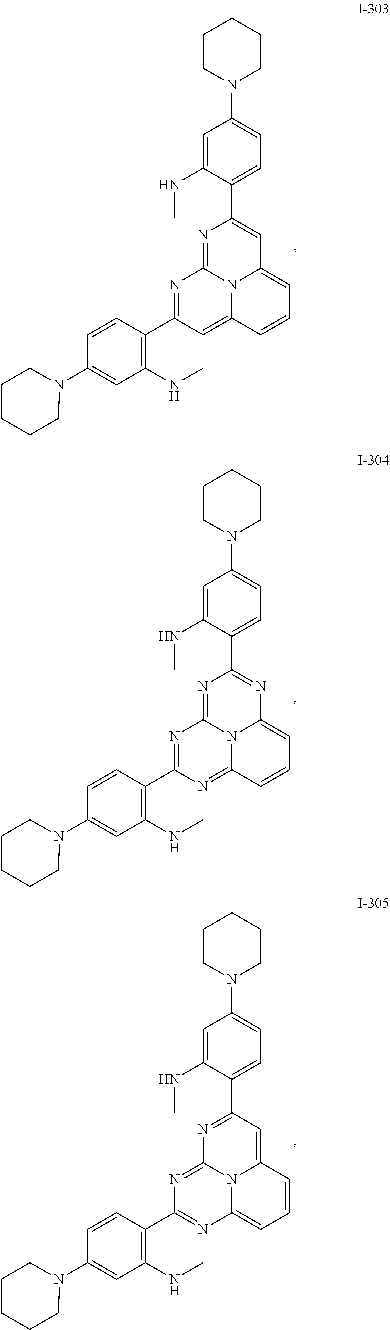 [SMILES: CNc1cc(N2CCCCC2)ccc1C1=CC2=CC=CC3=CC(c4ccc(N5CCCCC5)cc4NC)=NC(=N1)N23 ; CNc1cc(N2CCCCC2)ccc1C1=NC2=CC=CC3=NC(c4ccc(N5CCCCC5)cc4NC)=NC(=N1)N23 ; CNc1cc(N2CCCCC2)ccc1C1=NC2=NC(c3ccc(N4CCCCC4)cc3NC)=NC3=CC=CC(=C1)N32]

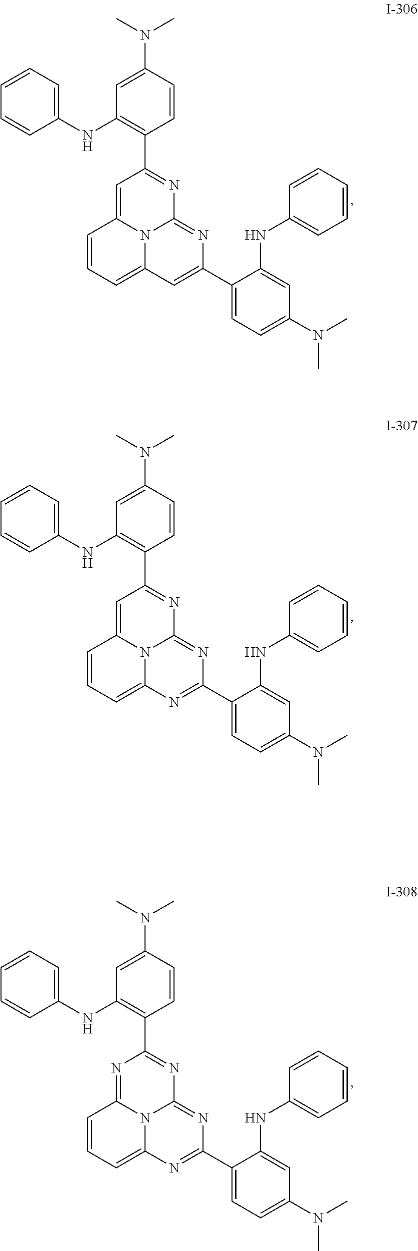 [SMILES: CN(C)c1ccc(C2=CC3=CC=CC4=CC(c5ccc(N(C)C)cc5Nc5ccccc5)=NC(=N2)N34)c(Nc2ccccc2)c1 ; CN(C)c1ccc(C2=NC3=NC(c4ccc(N(C)C)cc4Nc4ccccc4)=NC4=CC=CC(=C2)N43)c(Nc2ccccc2)c1 ; CN(C)c1ccc(C2=NC3=CC=CC4=NC(c5ccc(N(C)C)cc5Nc5ccccc5)=NC(=N2)N34)c(Nc2ccccc2)c1]

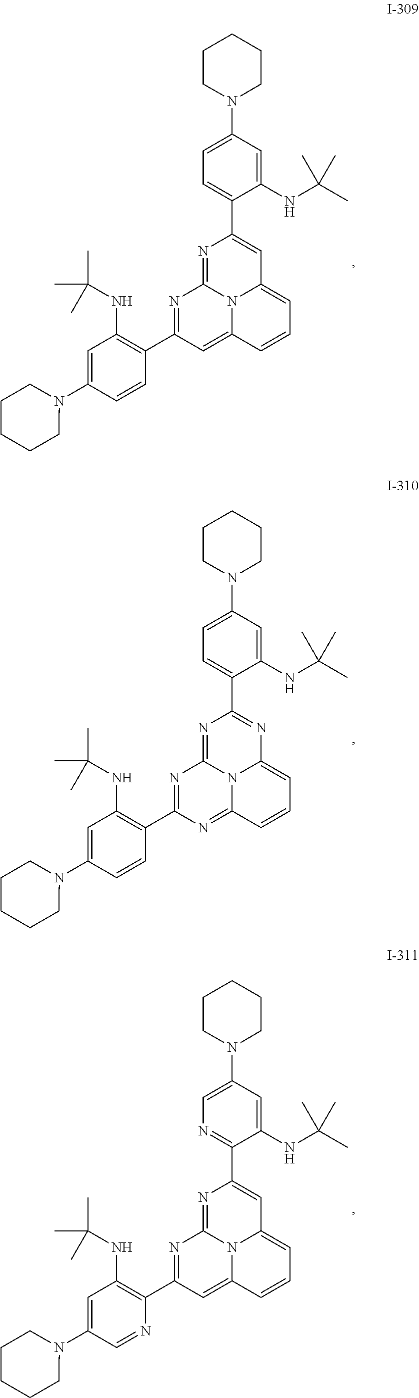 [SMILES: CC(C)(C)Nc1cc(N2CCCCC2)ccc1C1=CC2=CC=CC3=CC(c4ccc(N5CCCCC5)cc4NC(C)(C)C)=NC(=N1)N23 ; CC(C)(C)Nc1cc(N2CCCCC2)ccc1C1=NC2=CC=CC3=NC(c4ccc(N5CCCCC5)cc4NC(C)(C)C)=NC(=N1)N23 ; CC(C)(C)Nc1cc(N2CCCCC2)cnc1C1=CC2=CC=CC3=CC(c4ncc(N5CCCCC5)cc4NC(C)(C)C)=NC(=N1)N23]

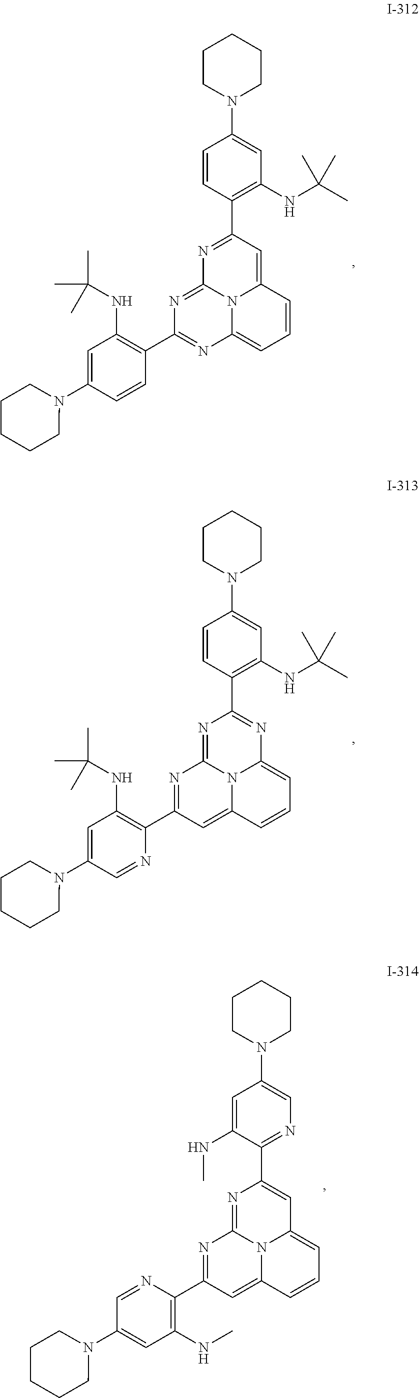 [SMILES: CC(C)(C)Nc1cc(N2CCCCC2)ccc1C1=NC2=NC(c3ccc(N4CCCCC4)cc3NC(C)(C)C)=NC3=CC=CC(=C1)N32 ; CC(C)(C)Nc1cc(N2CCCCC2)ccc1C1=NC2=CC=CC3=CC(c4ncc(N5CCCCC5)cc4NC(C)(C)C)=NC(=N1)N32 ; CNc1cc(N2CCCCC2)cnc1C1=CC2=CC=CC3=CC(c4ncc(N5CCCCC5)cc4NC)=NC(=N1)N23]

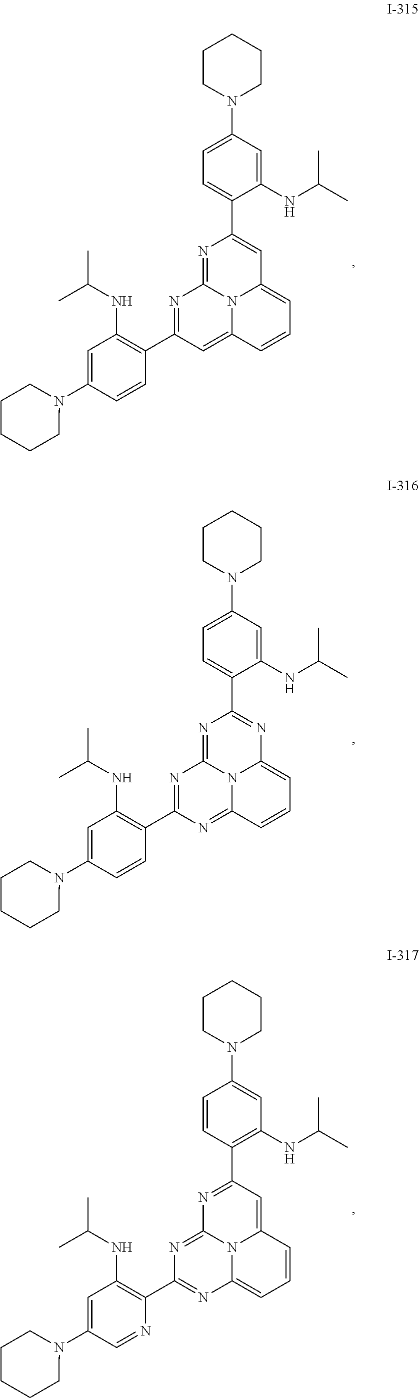 [SMILES: CC(C)Nc1cc(N2CCCCC2)ccc1C1=CC2=CC=CC3=CC(c4ccc(N5CCCCC5)cc4NC(C)C)=NC(=N1)N23 ; CC(C)Nc1cc(N2CCCCC2)ccc1C1=NC2=CC=CC3=NC(c4ccc(N5CCCCC5)cc4NC(C)C)=NC(=N1)N23 ; CC(C)Nc1cc(N2CCCCC2)ccc1C1=NC2=NC(c3ncc(N4CCCCC4)cc3NC(C)C)=NC3=CC=CC(=C1)N32]

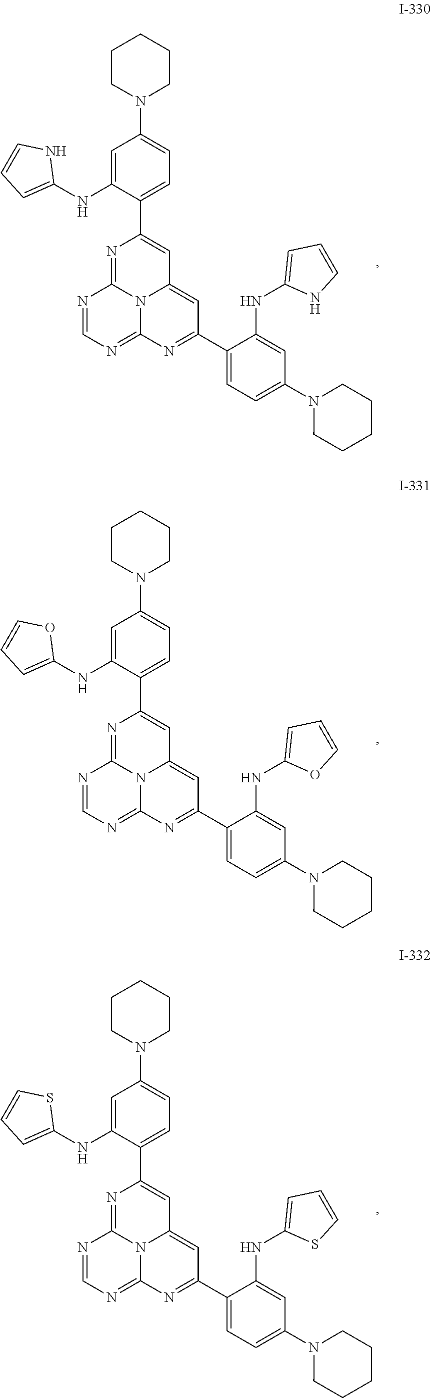 [SMILES: C1=NC2=NC(c3ccc(N4CCCCC4)cc3Nc3ccc[nH]3)=CC3=CC(c4ccc(N5CCCCC5)cc4Nc4ccc[nH]4)=NC(=N1)N32 ; C1=NC2=NC(c3ccc(N4CCCCC4)cc3Nc3ccco3)=CC3=CC(c4ccc(N5CCCCC5)cc4Nc4ccco4)=NC(=N1)N32 ; C1=NC2=NC(c3ccc(N4CCCCC4)cc3Nc3cccs3)=CC3=CC(c4ccc(N5CCCCC5)cc4Nc4cccs4)=NC(=N1)N32]

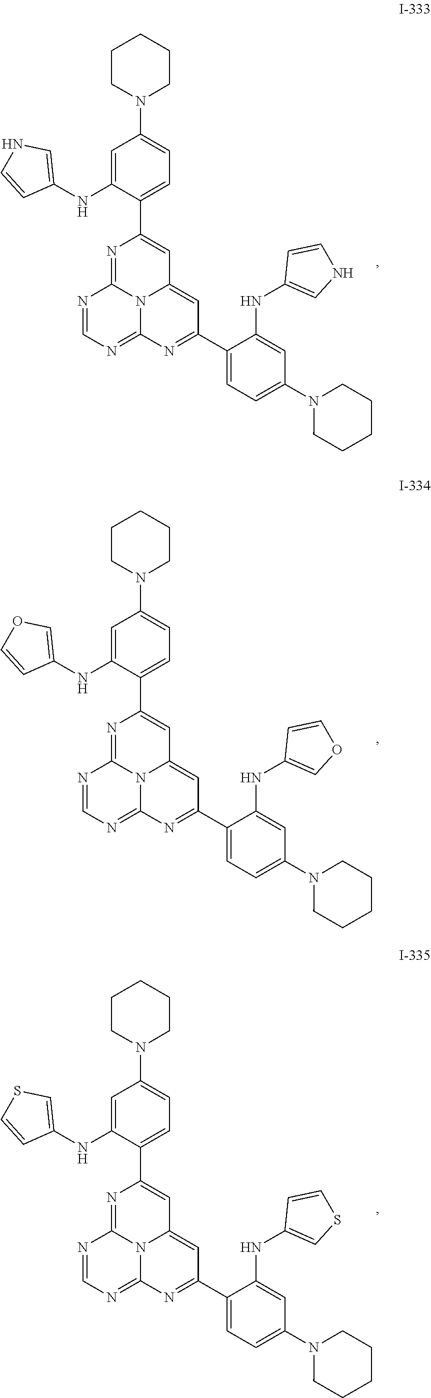 [SMILES: C1=NC2=NC(c3ccc(N4CCCCC4)cc3Nc3cc[nH]c3)=CC3=CC(c4ccc(N5CCCCC5)cc4Nc4cc[nH]c4)=NC(=N1)N32 ; C1=NC2=NC(c3ccc(N4CCCCC4)cc3Nc3ccoc3)=CC3=CC(c4ccc(N5CCCCC5)cc4Nc4ccoc4)=NC(=N1)N32 ; C1=NC2=NC(c3ccc(N4CCCCC4)cc3Nc3ccsc3)=CC3=CC(c4ccc(N5CCCCC5)cc4Nc4ccsc4)=NC(=N1)N32]

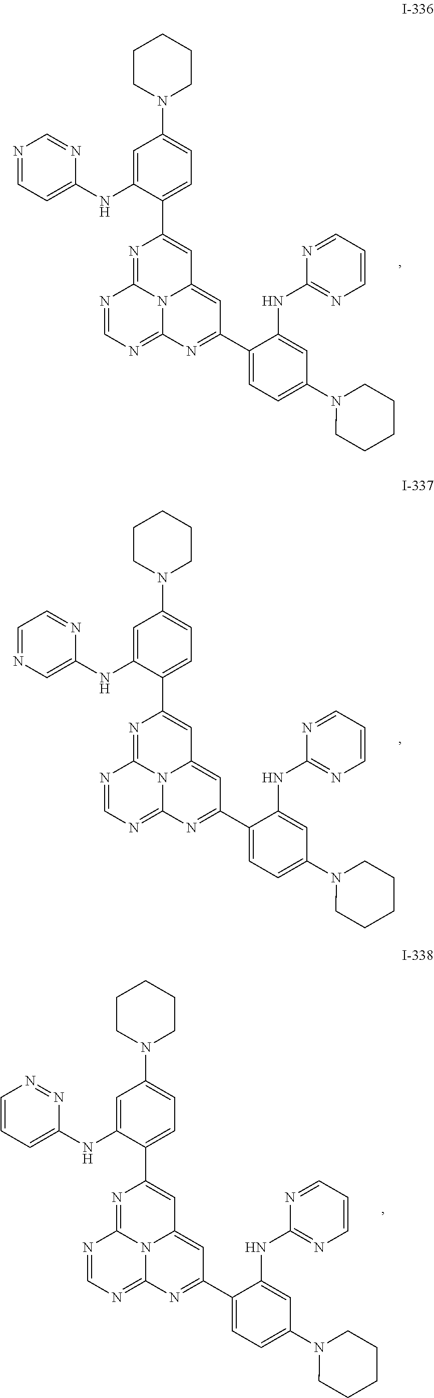 [SMILES: C1=NC2=NC(c3ccc(N4CCCCC4)cc3Nc3ccncn3)=CC3=CC(c4ccc(N5CCCCC5)cc4Nc4ncccn4)=NC(=N1)N32 ; C1=NC2=NC(c3ccc(N4CCCCC4)cc3Nc3cnccn3)=CC3=CC(c4ccc(N5CCCCC5)cc4Nc4ncccn4)=NC(=N1)N32 ; C1=NC2=NC(c3ccc(N4CCCCC4)cc3Nc3cccnn3)=CC3=CC(c4ccc(N5CCCCC5)cc4Nc4ncccn4)=NC(=N1)N32]

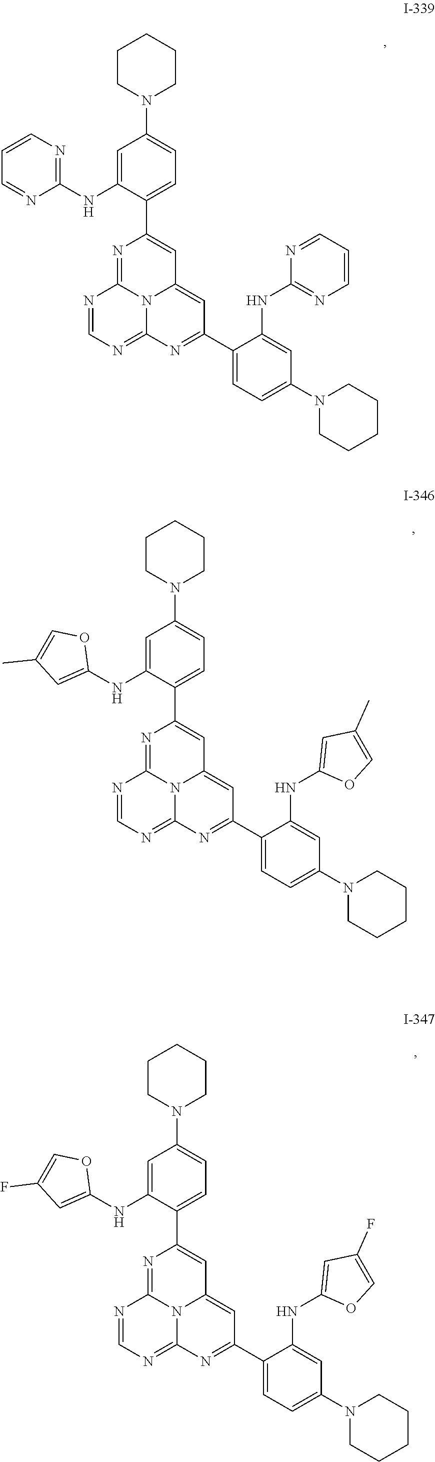 [SMILES: C1=NC2=NC(c3ccc(N4CCCCC4)cc3Nc3ncccn3)=CC3=CC(c4ccc(N5CCCCC5)cc4Nc4ncccn4)=NC(=N1)N32 ; Cc1coc(Nc2cc(N3CCCCC3)ccc2C2=CC3=CC(c4ccc(N5CCCCC5)cc4Nc4cc(C)co4)=NC4=NC=NC(=N2)N34)c1 ; Fc1coc(Nc2cc(N3CCCCC3)ccc2C2=CC3=CC(c4ccc(N5CCCCC5)cc4Nc4cc(F)co4)=NC4=NC=NC(=N2)N34)c1]

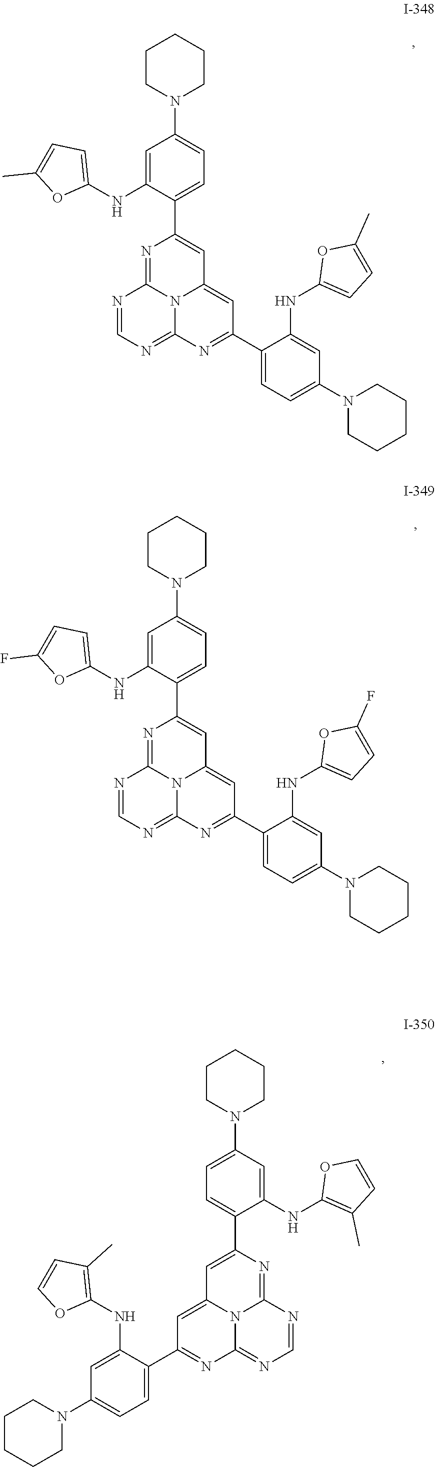 [SMILES: Cc1ccc(Nc2cc(N3CCCCC3)ccc2C2=CC3=CC(c4ccc(N5CCCCC5)cc4Nc4ccc(C)o4)=NC4=NC=NC(=N2)N34)o1 ; Fc1ccc(Nc2cc(N3CCCCC3)ccc2C2=CC3=CC(c4ccc(N5CCCCC5)cc4Nc4ccc(F)o4)=NC4=NC=NC(=N2)N34)o1 ; Cc1ccoc1Nc1cc(N2CCCCC2)ccc1C1=CC2=CC(c3ccc(N4CCCCC4)cc3Nc3occc3C)=NC3=NC=NC(=N1)N23]

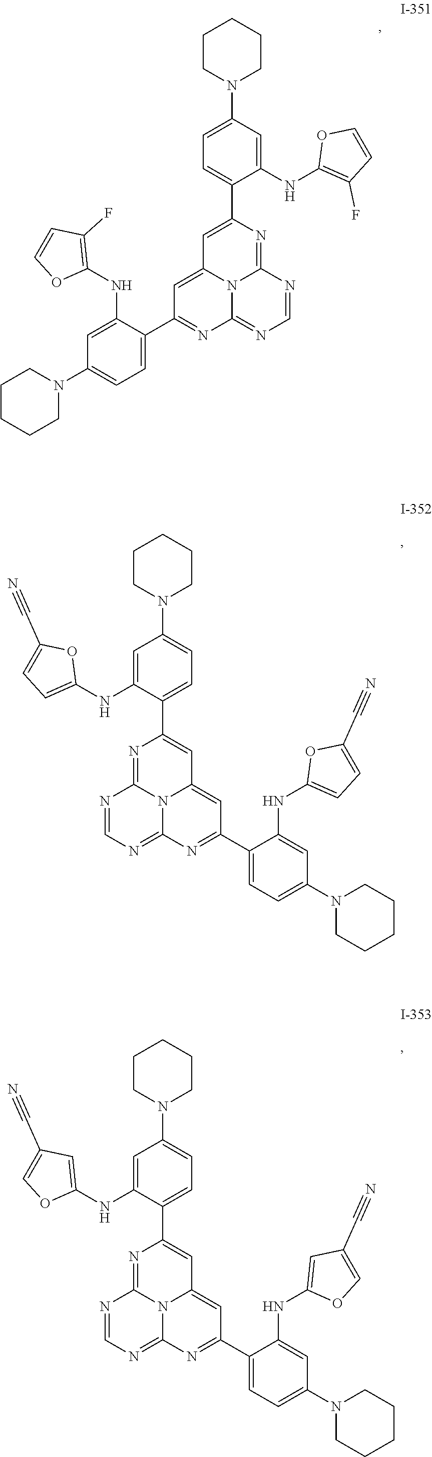 [SMILES: Fc1ccoc1Nc1cc(N2CCCCC2)ccc1C1=CC2=CC(c3ccc(N4CCCCC4)cc3Nc3occc3F)=NC3=NC=NC(=N1)N23 ; N#Cc1ccc(Nc2cc(N3CCCCC3)ccc2C2=CC3=CC(c4ccc(N5CCCCC5)cc4Nc4ccc(C#N)o4)=NC4=NC=NC(=N2)N34)o1 ; N#Cc1coc(Nc2cc(N3CCCCC3)ccc2C2=CC3=CC(c4ccc(N5CCCCC5)cc4Nc4cc(C#N)co4)=NC4=NC=NC(=N2)N34)c1]

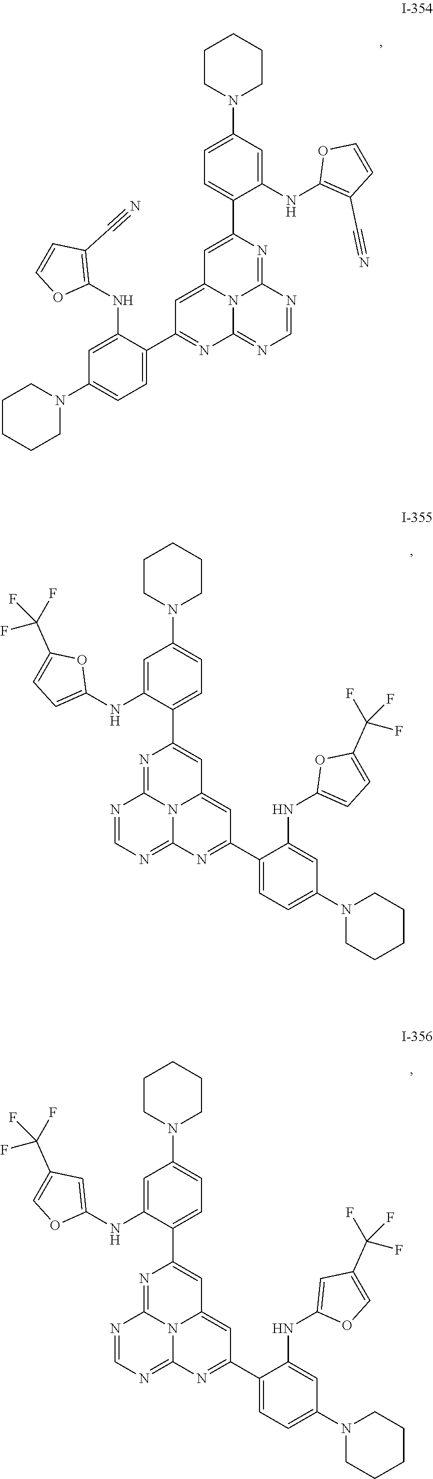 [SMILES: N#Cc1ccoc1Nc1cc(N2CCCCC2)ccc1C1=CC2=CC(c3ccc(N4CCCCC4)cc3Nc3occc3C#N)=NC3=NC=NC(=N1)N23 ; FC(F)(F)c1ccc(Nc2cc(N3CCCCC3)ccc2C2=CC3=CC(c4ccc(N5CCCCC5)cc4Nc4ccc(C(F)(F)F)o4)=NC4=NC=NC(=N2)N34)o1 ; FC(F)(F)c1coc(Nc2cc(N3CCCCC3)ccc2C2=CC3=CC(c4ccc(N5CCCCC5)cc4Nc4cc(C(F)(F)F)co4)=NC4=NC=NC(=N2)N34)c1]

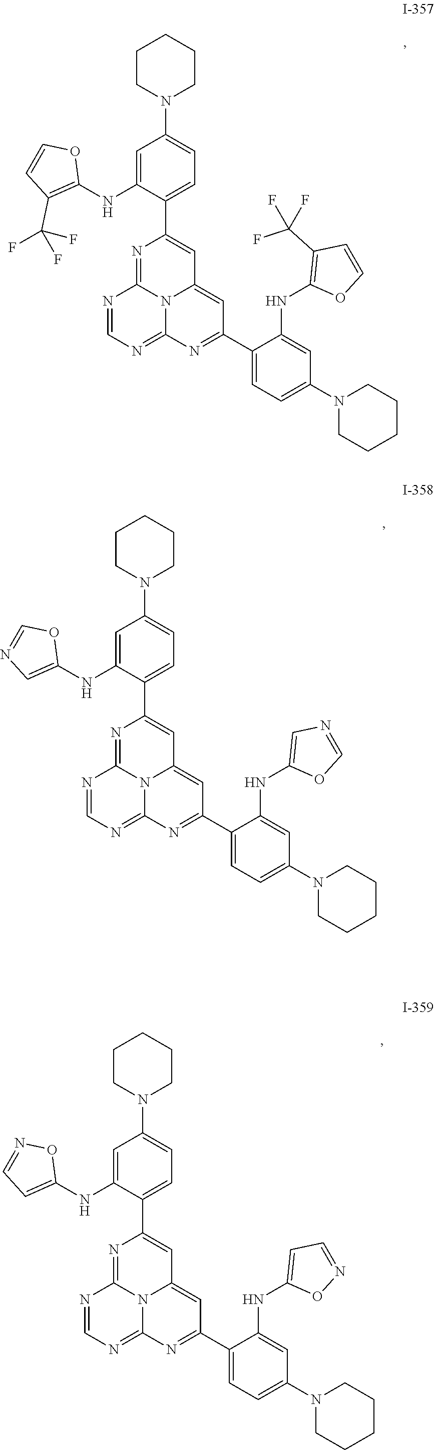 [SMILES: FC(F)(F)c1ccoc1Nc1cc(N2CCCCC2)ccc1C1=CC2=CC(c3ccc(N4CCCCC4)cc3Nc3occc3C(F)(F)F)=NC3=NC=NC(=N1)N23 ; C1=NC2=NC(c3ccc(N4CCCCC4)cc3Nc3cnco3)=CC3=CC(c4ccc(N5CCCCC5)cc4Nc4cnco4)=NC(=N1)N32 ; C1=NC2=NC(c3ccc(N4CCCCC4)cc3Nc3ccno3)=CC3=CC(c4ccc(N5CCCCC5)cc4Nc4ccno4)=NC(=N1)N32]

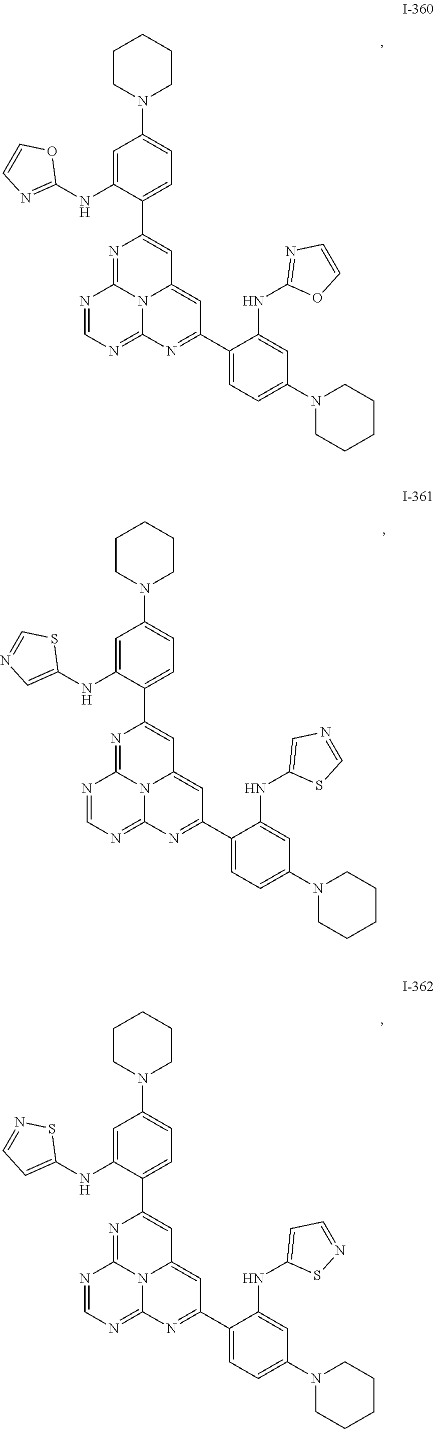 [SMILES: C1=NC2=NC(c3ccc(N4CCCCC4)cc3Nc3ncco3)=CC3=CC(c4ccc(N5CCCCC5)cc4Nc4ncco4)=NC(=N1)N32 ; C1=NC2=NC(c3ccc(N4CCCCC4)cc3Nc3cncs3)=CC3=CC(c4ccc(N5CCCCC5)cc4Nc4cncs4)=NC(=N1)N32 ; C1=NC2=NC(c3ccc(N4CCCCC4)cc3Nc3ccns3)=CC3=CC(c4ccc(N5CCCCC5)cc4Nc4ccns4)=NC(=N1)N32]

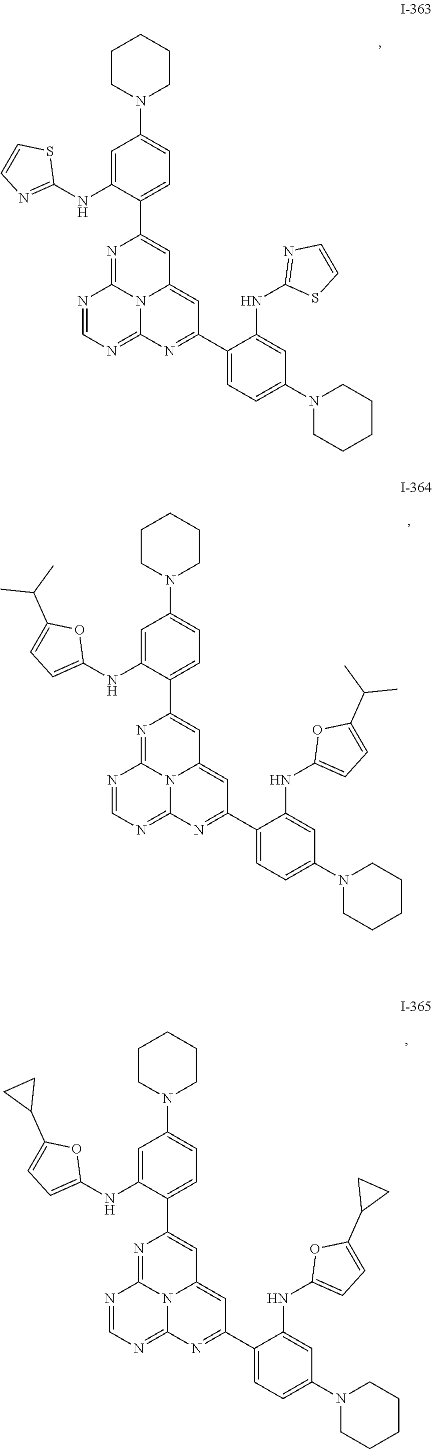 [SMILES: C1=NC2=NC(c3ccc(N4CCCCC4)cc3Nc3nccs3)=CC3=CC(c4ccc(N5CCCCC5)cc4Nc4nccs4)=NC(=N1)N32 ; CC(C)c1ccc(Nc2cc(N3CCCCC3)ccc2C2=CC3=CC(c4ccc(N5CCCCC5)cc4Nc4ccc(C(C)C)o4)=NC4=NC=NC(=N2)N34)o1 ; C1=NC2=NC(c3ccc(N4CCCCC4)cc3Nc3ccc(C4CC4)o3)=CC3=CC(c4ccc(N5CCCCC5)cc4Nc4ccc(C5CC5)o4)=NC(=N1)N32]

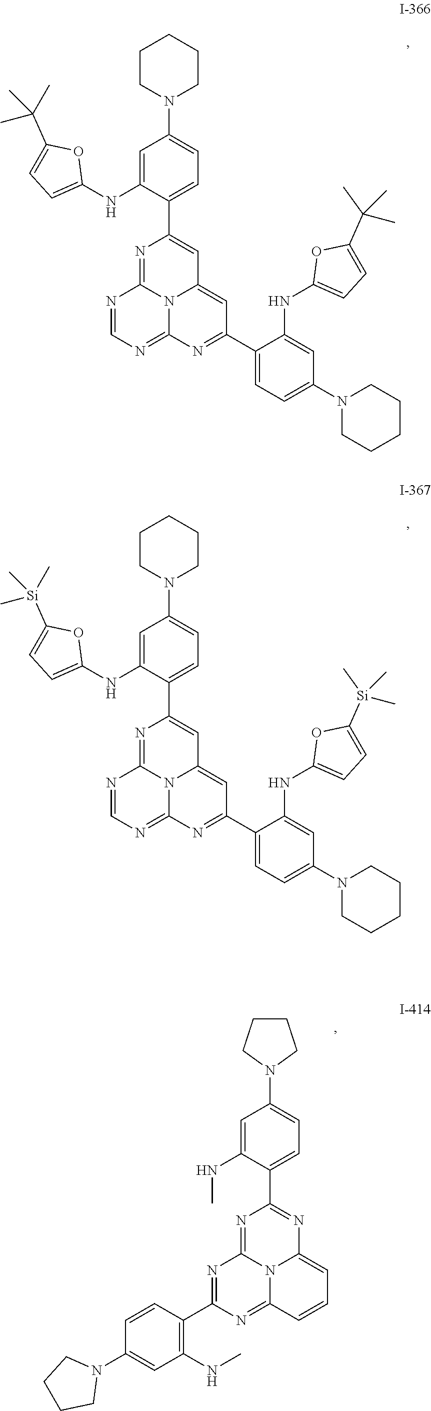 [SMILES: CC(C)(C)c1ccc(Nc2cc(N3CCCCC3)ccc2C2=CC3=CC(c4ccc(N5CCCCC5)cc4Nc4ccc(C(C)(C)C)o4)=NC4=NC=NC(=N2)N34)o1 ; C[Si](C)(C)c1ccc(Nc2cc(N3CCCCC3)ccc2C2=CC3=CC(c4ccc(N5CCCCC5)cc4Nc4ccc([Si](C)(C)C)o4)=NC4=NC=NC(=N2)N34)o1 ; CNc1cc(N2CCCC2)ccc1C1=NC2=CC=CC3=NC(c4ccc(N5CCCC5)cc4NC)=NC(=N1)N23]

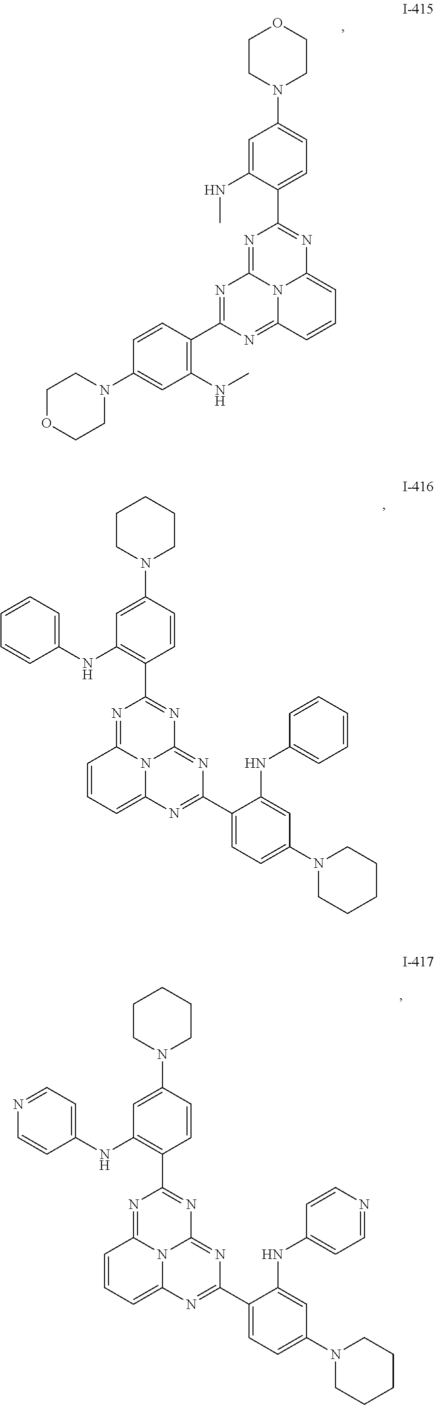 [SMILES: CNc1cc(N2CCOCC2)ccc1C1=NC2=CC=CC3=NC(c4ccc(N5CCOCC5)cc4NC)=NC(=N1)N23 ; C1=CC2=NC(c3ccc(N4CCCCC4)cc3Nc3ccccc3)=NC3=NC(c4ccc(N5CCCCC5)cc4Nc4ccccc4)=NC(=C1)N23 ; C1=CC2=NC(c3ccc(N4CCCCC4)cc3Nc3ccncc3)=NC3=NC(c4ccc(N5CCCCC5)cc4Nc4ccncc4)=NC(=C1)N23]

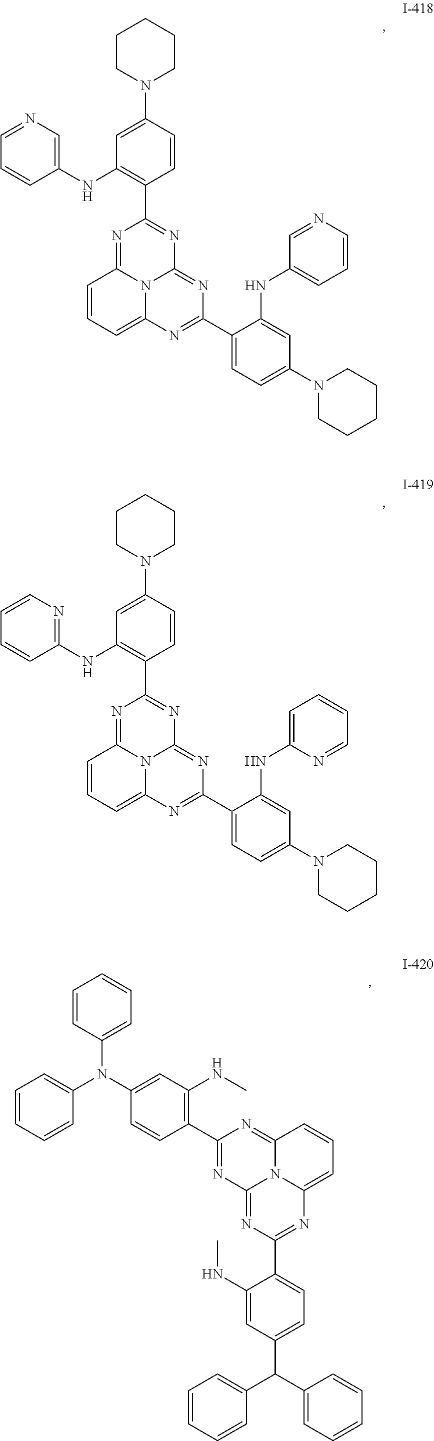 [SMILES: C1=CC2=NC(c3ccc(N4CCCCC4)cc3Nc3cccnc3)=NC3=NC(c4ccc(N5CCCCC5)cc4Nc4cccnc4)=NC(=C1)N23 ; C1=CC2=NC(c3ccc(N4CCCCC4)cc3Nc3ccccn3)=NC3=NC(c4ccc(N5CCCCC5)cc4Nc4ccccn4)=NC(=C1)N23 ; CNc1cc(C(c2ccccc2)c2ccccc2)ccc1C1=NC2=CC=CC3=NC(c4ccc(N(c5ccccc5)c5ccccc5)cc4NC)=NC(=N1)N23]

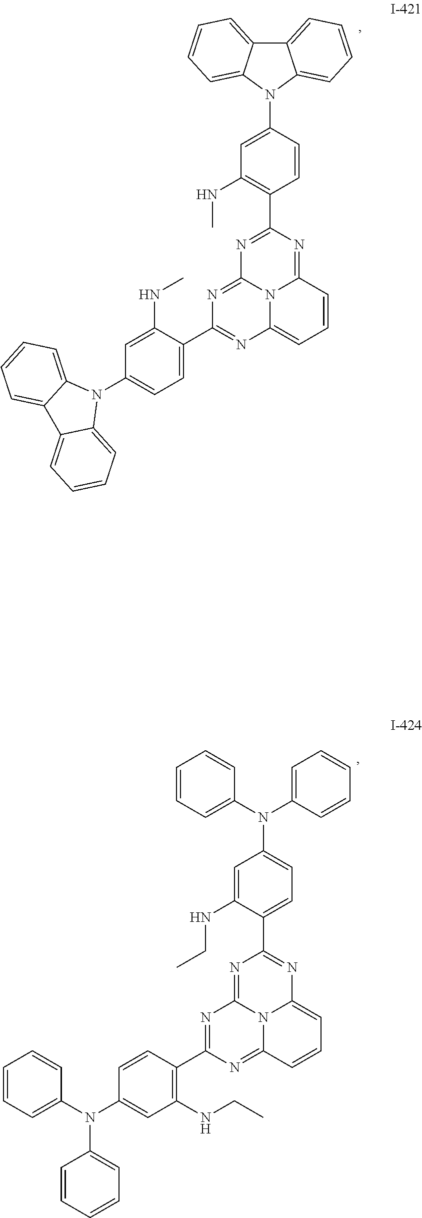 [SMILES: CNc1cc(-n2c3ccccc3c3ccccc32)ccc1C1=NC2=CC=CC3=NC(c4ccc(-n5c6ccccc6c6ccccc65)cc4NC)=NC(=N1)N23 ; CCNc1cc(N(c2ccccc2)c2ccccc2)ccc1C1=NC2=CC=CC3=NC(c4ccc(N(c5ccccc5)c5ccccc5)cc4NCC)=NC(=N1)N23]

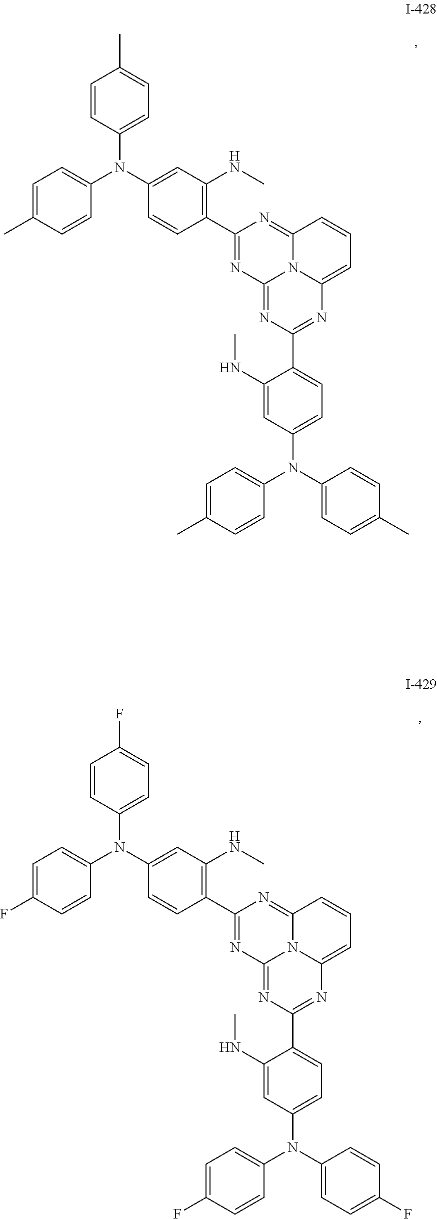

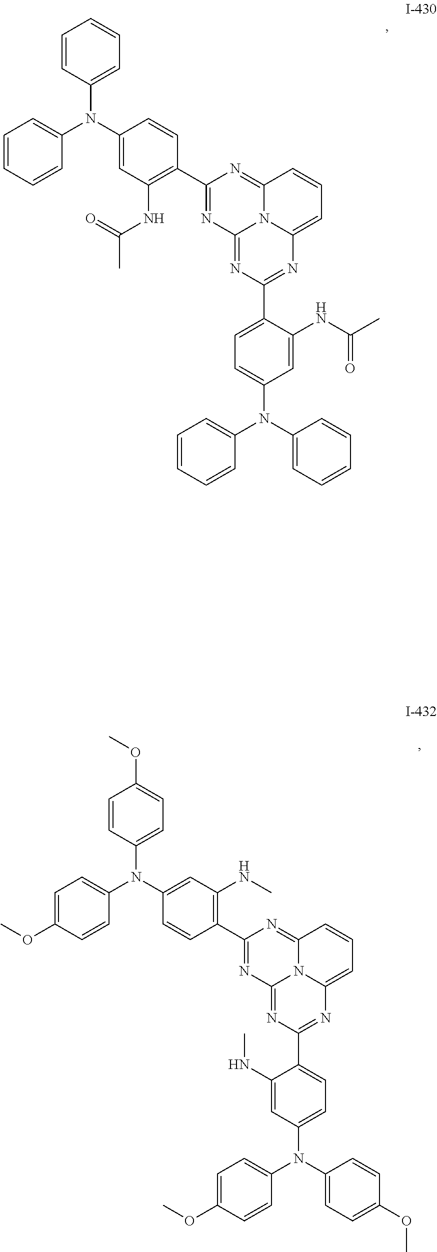

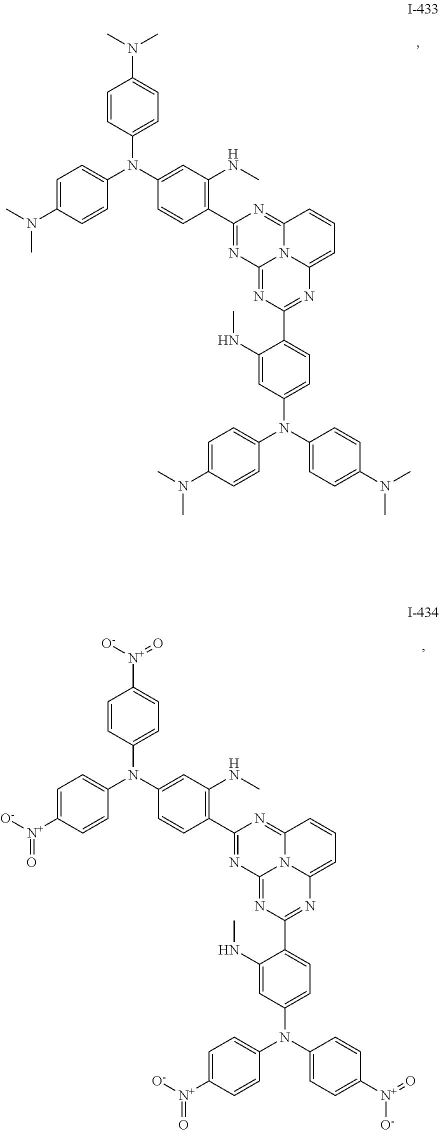

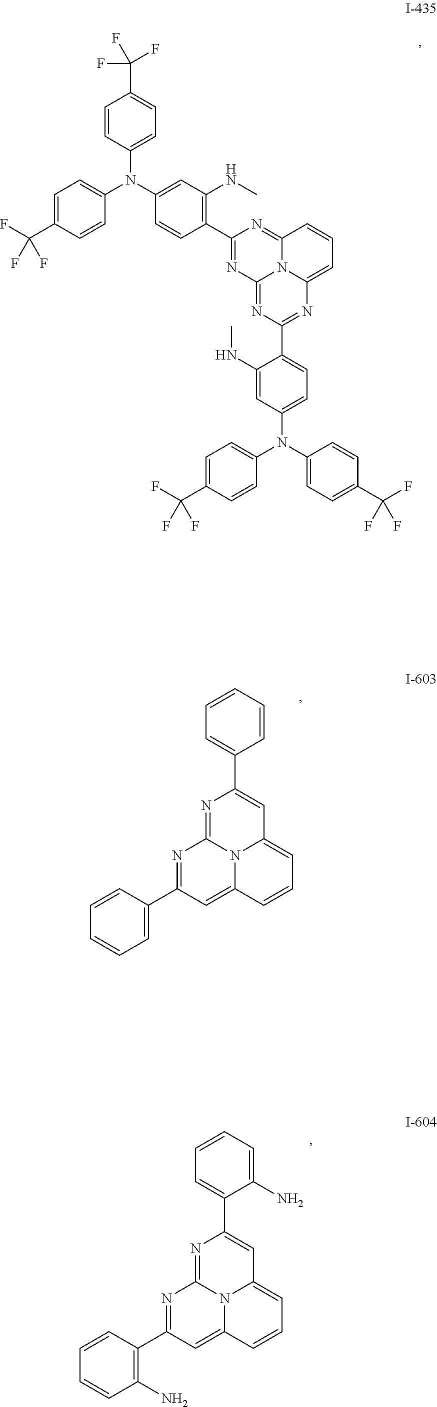 [SMILES: CNc1cc(N(c2ccc(C(F)(F)F)cc2)c2ccc(C(F)(F)F)cc2)ccc1C1=NC2=CC=CC3=NC(c4ccc(N(c5ccc(C(F)(F)F)cc5)c5ccc(C(F)(F)F)cc5)cc4NC)=NC(=N1)N23 ; C1=CC2=CC(c3ccccc3)=NC3=NC(c4ccccc4)=CC(=C1)N23 ; Nc1ccccc1C1=CC2=CC=CC3=CC(c4ccccc4N)=NC(=N1)N23]

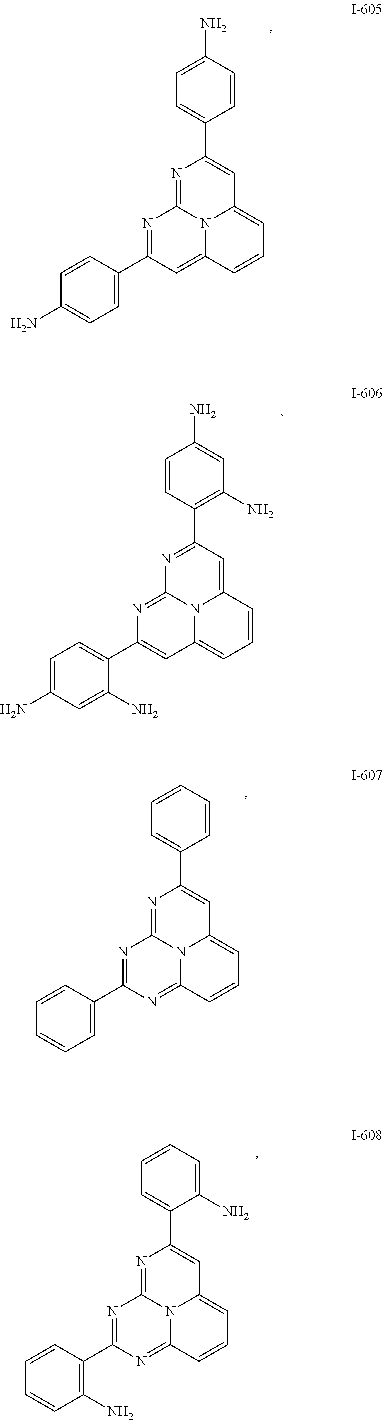 [SMILES: Nc1ccc(C2=CC3=CC=CC4=CC(c5ccc(N)cc5)=NC(=N2)N34)cc1 ; Nc1ccc(C2=CC3=CC=CC4=CC(c5ccc(N)cc5N)=NC(=N2)N34)c(N)c1 ; C1=CC2=NC(c3ccccc3)=NC3=NC(c4ccccc4)=CC(=C1)N23 ; Nc1ccccc1C1=CC2=CC=CC3=NC(c4ccccc4N)=NC(=N1)N23]

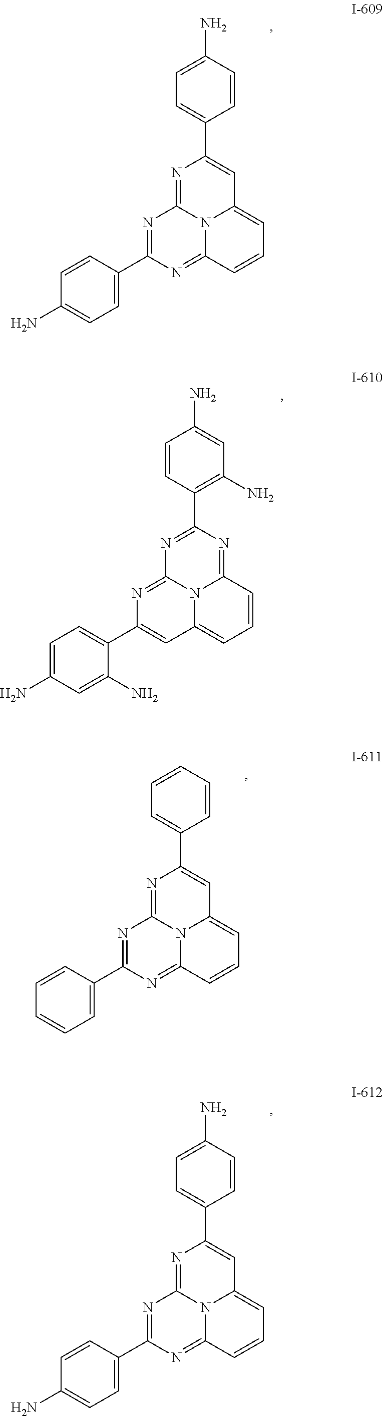 [SMILES: Nc1ccc(C2=CC3=CC=CC4=NC(c5ccc(N)cc5)=NC(=N2)N34)cc1 ; Nc1ccc(C2=CC3=CC=CC4=NC(c5ccc(N)cc5N)=NC(=N2)N34)c(N)c1 ; C1=CC2=NC(c3ccccc3)=NC3=NC(c4ccccc4)=CC(=C1)N23 ; Nc1ccc(C2=CC3=CC=CC4=NC(c5ccc(N)cc5)=NC(=N2)N34)cc1]

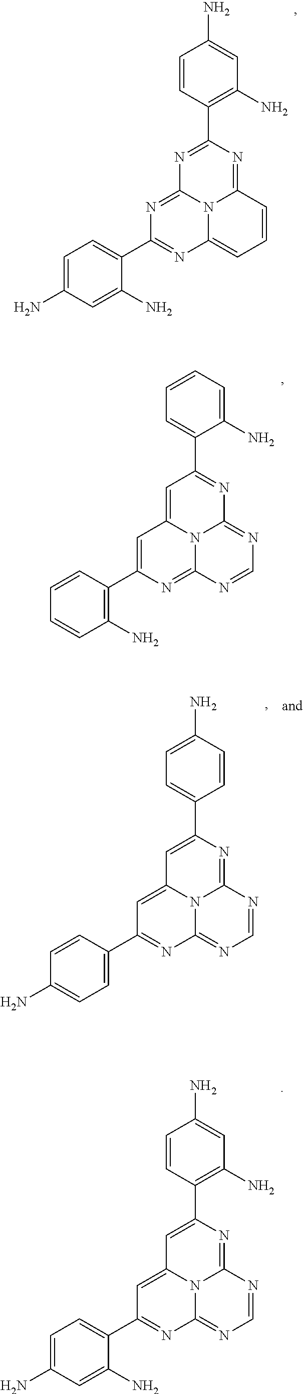 [SMILES: Nc1ccc(C2=NC3=CC=CC4=NC(c5ccc(N)cc5N)=NC(=N2)N34)c(N)c1 ; Nc1ccccc1C1=CC2=CC(c3ccccc3N)=NC3=NC=NC(=N1)N23 ; Nc1ccc(C2=CC3=CC(c4ccc(N)cc4)=NC4=NC=NC(=N2)N34)cc1 ; Nc1ccc(C2=CC3=CC(c4ccc(N)cc4N)=NC4=NC=NC(=N2)N34)c(N)c1]

28. Use of a compound of any one of claims 1 to 27 in an organic light-emitting diode.

29. The use of claim 28, wherein the compound is used as an emitter or a dopant.

30. An organic light-emitting diode comprising at least one compound of any one of claims 1 to 27.

31. Method of preparing an organic light-emitting diode comprising providing at least one compound of any one of claims 1 to 27 as an emitter or a dopant.

32. Use of a compound of any one of claims 1 to 27 as a photocatalyst.

33. Method of performing photocatalysis comprising contacting at least one compound of any one of claims 1 to 27 with a mixture requiring a photocatalyst and performing a photocatalytic transformation on the mixture.

34. Use of a compound of any one of claims 1 to 27 in the generation of organic laser.

35. Method of generating organic laser comprising providing at least one compound of any one of claims 1 to 27 as a light emitter.

36. Use of a compound of any one of claims 1 to 27 in the enhancement of photostability.

37. The use of claim 36, wherein the compound is used as a triplet quencher.

38. Method of enhancing photostability comprising providing at least one compound of any one of claims 1 to 27 as a triplet quencher.

39. A photocatalyst comprising at least one compound of any one of claims 1 to 27.

40. A triplet quencher comprising at least one compound of any one of claims 1 to 27.
